SEQUENCE LISTING (194 sequences)

SEQ ID NO: 1 - protein, 133 aa, Homo sapiens [sequence omitted]

SEQ ID NO: 2 - protein, 133 aa, Artificial Sequence
  source: /note=“Description of Artificial Sequence: Synthetic polypeptide”
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 3 - protein, 133 aa, Artificial Sequence
  source: /note=“Description of Artificial Sequence: Synthetic polypeptide”
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 4 - protein, 133 aa, Artificial Sequence
  source: /note=“Description of Artificial Sequence: Synthetic polypeptide”
APTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 5 - protein, 133 aa, Artificial Sequence
  source: /note=“Description of Artificial Sequence: Synthetic polypeptide”
APTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 6 - protein, 133 aa, Artificial Sequence
  source: /note=“Description of Artificial Sequence: Synthetic polypeptide”
APTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - RNA, 822 nt, Homo sapiens
aguucccuaucacucucuuuaaucacuacucacaguaaccucaacuccugccacaauguacaggaugcaacuccugucuugcauugcacuaagucuugcacuugucacaaacagugcaccuacuucaaguucuacaaagaaaacacagcuacaacuggagcauuuacugcuggauuuacagaugauuuugaauggaauuaauaauuacaagaaucccaaacucaccaggaugcucacauuuaaguuuuacaugcccaagaaggccacagaacugaaacaucuucagugucuagaagaagaacucaaaccucuggaggaagugcuaaauuuagcucaaagcaaaaacuuucacuuaagacccagggacuuaaucagcaauaucaacguaauaguucuggaacuaaagggaucugaaacaacauucaugugugaauaugcugaugagacagcaaccauuguagaauuucugaacagauggauuaccuuuugucaaagcaucaucucaacacugacuugauaauuaagugcuucccacuuaaaacauaucaggccuucuauuuauuuaaauauuuaaauuuuauauuuauuguugaauguaugguuugcuaccuauuguaacuauuauucuuaaucuuaaaacuauaaauauggaucuuuuaugauucuuuuuguaagcccuaggggcucuaaaaugguuucacuuauuuaucccaaaauauuuauuauuauguugaauguuaaauauaguaucuauguagauugguuaguaaaacuauuuaauaaauuugauaaauauaaaaaaaaaaaaaaaaaaaaaaaaaa

SEQ ID NO: 8 - protein, 584 aa, Homo sapiens
AHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 746 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 746 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 11 - protein, 746 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 12 - protein, 746 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 13 - protein, 746 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 14 - protein, 127 aa, Homo sapiens [sequence omitted]

SEQ ID NO: 15 - RNA, 788 nt, Homo sapiens
aguacacagagagaaaggcuaaaguucucuggaggauguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggagugagaccggccagaugaggcuggccaagccggggagcugcucucucaugaaacaagagcuagaaacucaggauggucaucuuggagggaccaaggggugggccacagccauggugggaguggccuggaccugcccugggccacacugacccugauacaggcauggcagaagaaugggaauauuuuauacugacagaaaucaguaauauuuauauauuuauauuuuuaaaauauuuauuuauuuauuuauuuaaguucauauuccauauuuauucaagauguuuuaccguaauaauuauuauuaaaaauaugcuucuacuug

SEQ ID NO: 16 - protein, 740 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 17 - protein, 118 aa, Artificial Sequence
  source: /note=“Description of Artificial Sequence: Synthetic polypeptide”
EVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSS

SEQ ID NO: 18 - protein, 884 aa, Artificial Sequence
  source: /note=“Description of Artificial Sequence: Synthetic polypeptide”
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 19 - protein, 884 aa, Artificial Sequence
  source: /note=“Description of Artificial Sequence: Synthetic polypeptide”
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 20 - protein, 883 aa, Artificial Sequence
  source: /note=“Description of Artificial Sequence: Synthetic polypeptide”
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGKPIPNPLLGLDS

SEQ ID NO: 21 - RNA, 2652 nt, Artificial Sequence
  source: /note=“Description of Artificial Sequence: Synthetic polynucleotide”
augccucuguuacugcugcucccacuucugugggccggcgcucuggcugaggugcagcuugugcagaccggcggaggucugucacaguucggcgaaucccuaaggcucucuugcgccgugucugguuucaacgugagcaacaacuauaugaguuggguucgccaggcuccaggcaagggucuggaguggguguccauuaucuauuccggcgguggcacacauuacgccgacucagucaaggguagauucacuaucaguagagacaacucaaagaacacucuuuuccuacaaaugaacagcuugcgggcagaggauacggccguguauuauugugcccgagcugucccagugccucacggaacagauauuuggggacaagguacaauggugaccgucucuuccggcggaggaggcucuggaggcggaggaucuggcggaggcggcagcgcccauaagagcgagguagcccauagauucaaggaccuuggcgaagagaacuucaaggccuuaguucucaucgccuucgcccaauacuugcagcaguguccauucgaagaucacguuaagcuugugaacgaggugaccgaauucgcuaagacgugcguggccgacgaguccgcugagaauugugauaagucacugcacacuuuguucggcgacaagcugugcaccgucgccacccugcgcgaaacauacggcgaaauggcggacuguugugcuaagcaggagccagaacggaacgaguguuuccugcagcauaaggacgacaauccaaaccucccuagguuggugcgcccagaaguggacgucauguguaccgcuuuccacgauaacgaggagacauuccuuaagaaguaucuguacgagauugcacggcggcacccuuacuucuacgccccugagcuccuguucuucgcaaagcgguauaaggcugcauucaccgaguguugccaggcggcagacaaggccgccugcuugcucccuaagcuugacgaacuuagggacgagggaaaggcuaguagcgccaagcagagacucaagugugcaucgcuacagaaguucggagagagagcuuucaaggcgugggcugucgcgcgccugucccagagguucccaaaggccgaauucgccgaggugagcaaguuagugacagaucucaccaaggugcacacugaguguugccacggcgacuuauuggagugugcagacgaucgggcugacuuggcaaaguacaucugugagaaccaggacucaauuuccaguaagcugaaggaguguugcgagaagccacucuuggagaagucccauuguauagccgaaguggagaacgacgagaugccggcugaccugccgucacuggcagcugauuucguagagucgaaggacguguguaagaacuacgcugaagcuaaggacguauuccugggaauguuccuuuacgaguacgcccgacggcauccagacuacucaguagugcugcuucugagacuggccaagacuuacgagacuacccuggagaaguguugugcagcggccgauccacacgaguguuacgccaagguguucgacgaauucaagccucugguggaggagccgcagaaccugaucaagcagaauugcgaacuguucgagcaguugggagaauauaaguuccagaacgcacugcuagugcgguauaccaagaaggucccacagguauccacuccgacacugguggaaguguccaggaaccucggcaaggugggaagcaagugcuguaagcacccggaggcuaagcggaugccgugcgccgaagacuaccucagcgugguccuuaaucagcugugcgugcugcacgagaagaccccaguaagugaccggguuacgaagugcuguacugaaucacuugugaacagaagaccuuguuucuccgcccucgagguggacgaaacguacguuccgaaggaauucaacgccgaaacauucaccuuccacgcugacauuuguacgcuuuccgagaaggagagacaaaucaagaagcagacugcccugguggaauuagugaagcacaagccgaaggccacaaaggagcagcugaaggcaguuauggacgacuucgcugcauucguagagaagugcugcaaggcagacgacaaggaaacuugcuucgcagaggagggaaagaagcucguugcugcgucucaggcggcucugggacugggaggaggauccgcuccuacaagcaguuccacaaagaagacccagcuccagcuugaacaucugcuucucgaccugcaaaugauccucaacgguaucaauaauuacaagaauccuaaguugacucgcaugcugacuuucaaguucuauaugccaaagaaggccacugaguugaagcaccugcagugccucgaggaagaacugaagccucuggaggaagucuuaaaccuggcacaaagcaagaacuuccaucugaggccuagggaccucauuuccaauaucaacguuauugugcuggagcucaaggguagcgagacgacauucaugugcgaguacgcagacgagacagcgacuaucgucgaguuccuaaauagguggauaacguucugccagucaaucaucuccacucugaccggcaagccgaucccuaacccguugcuuggauuggauucuaca

SEQ ID NO: 22 - RNA, 2652 nt, Artificial Sequence
  source: /note=“Description of Artificial Sequence: Synthetic polynucleotide”
augccucugcugcucuuacugccgcugcuuugggcaggagcucuggccgagguccagcuagugcagacaggcgguggacucagccaguucggugagucacugcgucuauccugcgcgguuaguggcuucaacguguccaacaauuauaugaguugggugagacaagcaccuggcaagggccuugaguggguguccauuauauauagcgguggcggcacccacuacgcugacucggugaagggucgauucacaauuagcagagacaauuccaagaauacucuguuccugcagaugaacucucucagggcggaggacaccgccguguacuacugugcccgugcggugccggucccucacgguacagacaucuggggccaggguaccauggucaccgugucauccggaggaggcgguucugguggaggaggaaguggaggcggcggcagugcacauaagagcgagguggcccauagauucaaggauuuaggagaggagaacuucaaggcacuggucuugaucgccuucgcacaauaucuucagcaguguccguucgaagaucacgugaagcucgucaacgaggugacugaguucgccaagaccugcguggcagacgagucggcugagaauugugauaagagucugcacacccucuucggagauaagcuguguaccguggccacucugagggagacauacggcgagauggcugacuguugugcaaagcaggagccagagaggaacgaguguuucuugcagcauaaggacgacaacccaaaucugccucgucugguuagaccugaaguggacguaaugugcaccgcuuuccacgacaacgaggagacguuccugaagaaguaucuguacgagauugcuagaaggcacccauacuucuacgcaccagagcuguuguucuucgcuaagagauacaaggccgcuuucaccgagugcugccaagcagcagacaaggcugcuugccugcucccuaagcuggacgagcugagagacgaggguaaggcuucaucagccaagcaaagauugaagugugcaucacugcagaaguucggcgaaagagcauucaaggcuugggccgucgccaggcugucucagagguucccgaaggccgaguucgcugaagugucuaaguuagugaccgaucuaacaaaggugcacacagaguguugccacggagaucugcuugagugcgccgacgaucgagccgaucuugcaaaguacaucugugagaaccaggauuccaucuccucuaaguugaaggagugcugugagaagccacuucucgagaagucucacugcaucgcagaagucgagaacgacgaaaugccagcugaccugccgagcuuggccgcagauuucgucgagaguaaggacguuugcaagaauuacgcugaggcuaaggacgucuuccugggcauguucuuguacgaauacgccaggaggcacccggauuacucagugguacuuuuacugcgacuggcuaagaccuacgaaacgacacuggagaagugcugcgcagccgccgauccucacgagugcuacgcuaagguuuucgacgaguucaagccgcucguggaagaaccucagaacuuaaucaagcagaauugugagcuauucgaacagcucggagaguauaaguuccagaacgcccuccuggugcgguacacaaagaagguuccacaggucagcaccccgacccucguugaggugagccgcaaccucggcaagguuggcucuaaguguuguaagcauccggaagcgaagcgcaugccuugugcagaagauuaucuuucaguuguguugaaccagcucuguguccugcacgagaagaccccaguuuccgaccgcguuaccaaguguugcacagaaagcuuggucaaccggaggccuugcuucagcgcauuggagguagacgaaaccuacgucccuaaggaauucaacgccgaaacauucacguuccacgcggacauuugcacacucuccgagaaggaacgccagauuaagaagcagacugccuugguugagcugguaaagcacaagccuaaggcgacuaaggagcagcuaaaggcaguuauggacgacuucgcugcauucguugagaagugcuguaaggccgacgauaaggaaaccugcuucgccgaggaaggaaagaagcuuguugccgcuagccaggccgcacugggauuggguggaggcagcgcaccaacuuccucuagcaccaagaagacucaauuacaacuggaacaucucuuacuaacacuccaaaugauacugaacggcauaaacaacuauaagaacccgaaguugaccagaauguuaaccuucaaguucuacaugccuaagaaggcuacggaacugaagcaccuucagugccuagaggaggagcucaagccucucgaggagguucugaaccucgcacagaguaagaauuuccaccugcgcccaagggaccugauuuccaauauuaacgugaucguccuugaacuuaagggcagcgaaaccacuuucaugugcgaauacgcggacgaaacugcuaccauaguugaguuccuuaaccgcuggaucacauucugccagucaaucaucucuacauugaccggaaagccgauuccgaacccacuccugggacuugauagcaca

SEQ ID NO: 23 - RNA, 2652 nt, Artificial Sequence
  source: /note=“Description of Artificial Sequence: Synthetic polynucleotide”
augccucugcuccugcuucuuccucugcucugggcuggcgcccuagccgaggugcagcuggugcagaccggcggcggccugagccaguucggcgagagccugagacugagcugcgccgugagcggcuucaacgugagcaacaacuacaugagcugggugagacaggccccuggcaagggccuggagugggugagcaucaucuacaguggcggagguacccacuacgccgacagcgugaagggcagauucaccaucagcagagacaacagcaagaacacccuguuccugcagaugaacagccucagggccgaggacaccgccguguacuacugcgccagagccgugccugugccucacggcaccgacaucuggggccagggcaccauggugaccgugaguagcgguggaggcggaagcggaggcgguggcucuggagguggcggcagcgcccacaagagcgagguggcccacagauucaaggaccugggcgaggagaacuucaaggcccuggugcugaucgccuucgcccaguaccugcagcagugcccuuucgaggaccacgugaagcuggugaacgaggugaccgaguucgccaagaccugcguggccgacgagagcgccgagaacugcgacaagagccugcacacacucuucggcgacaagcugugcaccguggccacccugagagagacuuacggcgagauggccgacugcugcgccaagcaggaaccagagaggaacgagugcuuccuucagcacaaggacgacaacccuaaccugccuagacugguucggccugagguggacgugaugugcacugccuuccacgacaacgaggagacguuccugaagaaguaccuguacgagaucgccagaagacacccuuacuucuacgccccugagcuucuguucuucgcaaagagauacaaggccgccuucaccgagugcugccaggccgccgacaaggcugccugccuccugccaaagcuggacgagcugagagacgagggcaaggccagcucugcgaagcagaggcugaagugcgccagccugcagaaguucggagaaagagcuuucaaggccugggccguggcccgcuugucucaaagauucccuaaggcggaguucgcagaagugagcaagcuggucaccgaccugaccaaggugcauacugaguguugccacggcgaccugcuggagugcgccgacgacagagccgaccuggccaaguacaucugcgagaaccaggacucuauuaguagcaaguugaaggaguguugcgagaagccgcuacucgagaagagucacugcauugcggagguugagaacgacgagaugccugccgaccucccaagccuggcagccgauuucguggagagcaaggacgugugcaagaauuacgcugaggcuaaggacguuuuccugggcauguucuuauacgaauacgcuagacggcacccagacuacagcgugguccuccuccugcggcuugcaaagacguacgaaacuacccuggagaagugcugcgcggcugcugacccucacgagugcuacgccaagguguucgacgaguucaagccucucguggaggagccucagaaccugaucaagcagaauugcgagcuguucgagcaguuaggugaguacaaguuccagaacgcccugcuugugcgcuacaccaagaaggugccucagguuuccaccccuacccugguagagguguccagaaaccugggcaaggugggcagcaaguguugcaagcauccagaagcuaagagaaugccuugcgccgaagauuaccugucugucgugcugaaccagcugugcgugcugcacgagaagaccccugugagcgacagagugaccaagugcuguacagaaucccuaguaaauagaagaccuugcuucuccgcccuagagguggacgaaaccuacgugccuaaggaguucaacgccgagacuuucacguuccacgcggauauuuguacacucagcgagaaggagagacagaucaagaagcagaccgcacuuguggagcucgucaagcacaagccaaaggccaccaaggagcaacucaaggccgugauggacgacuucgcagccuucgucgagaaguguuguaaggcugacgauaaggaaaccugcuucgcugaggaaggcaagaagcuuguggccgccagucaggcugcucuuggucuuggcggcggaucagcaccuaccagcagcucuacaaagaagacgcagcugcaacuggagcacuugcugcuugaucugcaaaugauccugaacggcaucaacaauuacaagaacccuaagcugaccagaaugcugaccuucaaguucuacaugccuaagaaggccacagagcugaagcauuuacagugccuggaggaggagcuuaagccacuugaagaggugcucaaucuggcccagucuaagaacuuccaccucaggccucgcgaucugauuagcgacaucaacguaaucgugcuggaacuuaagggaucugaaacuacguucaugugcgaguacgccgacgaaacagccaccaucguggaguuccugaaccgauggaucaccuucugccagaguauuauuucaacccucaccggcaagccuaucccuaacccacugcucggacucgacagcacc

SEQ ID NO: 24 - RNA, 2220 nt, Artificial Sequence
  source: /note=“Description of Artificial Sequence: Synthetic polynucleotide”
augaagugggugaccuucaucagccugcuguuccuguucagcagcgccuacagcagaggcguguucagaagagacgcccacaagagcgagguggcccacagauucaaggaccugggcgaggagaacuucaaggcccuggugcugaucgccuucgcccaguaccugcagcagugcccuuucgaggaccacgugaagcuggugaacgaggugaccgaguucgccaagaccugcguggccgacgagagcgccgagaacugcgacaagagccugcacacccuguucggcgacaagcugugcaccguggccacccugagagaaacuuacggcgagauggccgacugcugcgccaagcaggagccagagcggaacgagugcuuccugcaacacaaggacgacaacccuaaccugccuagacugguccggccugagguggacgugaugugcacggccuuccacgacaacgaggagacuuuccugaagaaguaccuguacgagaucgccagaagacacccuuacuucuacgccccugagcuccuguucuucgcgaagagauacaaggccgccuucaccgagugcugccaggccgccgacaaggcagcuugccugcugccuaagcuggacgagcugagagacgagggcaaggccuccucagcuaagcagagacugaagugcgccagccugcagaaguucggugagagagcauucaaggcuugggccgucgcaagacugucacagagauucccuaaggcagaauucgcggaggugagcaagcuagugaccgaccugaccaaggugcauacagagugcugccacggcgaccugcuggagugcgccgacgacagagccgaccuggccaaguacaucugcgagaaccaggacagcaucagcuccaagcugaaggagugcugugagaagccucugcuggagaagucacacugcauugccgaggucgagaacgacgagaugccugccgaucuuccuagccuugccgccgauuucguggagagcaaggacgugugcaagaacuacgccgaggcaaaggacguguuccugggcauguuccuuuacgaauacgcucgccggcauccagacuacagcguggugcugcugcugagauuggccaagacuuacgagacgacccucgagaaguguugcgcagcagcugauccucacgaguguuacgccaagguguucgacgaguucaagccgcuuguggaggagccucagaaccugaucaagcagaauugugagcuguucgagcagcugggugaguacaaguuccagaacgcccugcuggugcgcuacaccaagaaggugccucaagugucuaccccuacccugguugaaguuucccgcaaccugggcaaggugggcagcaagugcugcaagcauccugaagcaaagaggaugccuugcgccgaggacuaccugucagugguccuuaaccagcugugcgugcugcacgagaagaccccugugagcgacagagugacaaaguguuguaccgagagccuggucaacagaagaccuugcuucagcgcccuggaagucgacgagacauacgugccuaaggaguucaacgccgaaaccuucaccuuccacgccgacaucugcacacugagcgagaaggagagacagaucaagaagcagaccgcccuggucgaguuggugaagcacaagccuaaggccaccaaggagcaacucaaggccgugauggacgacuucgcggccuucguugagaagugcuguaaggcugacgacaaggagacgugcuucgcugaggaggguaagaagcuugucgccgccucucaggccgcuuugggacucggcggcggcagugcgccugccagaagcccuuccccaucuacccagccuugggagcacgugaacgccauccaggaggccagacgucugcugaaccugucacgggauaccgcagcugagaugaacgaaacuguugaggucaucagcgagauguucgaccuacaggaaccuaccugcuugcagaccagacuggagcuguacaagcagggauuaagaggcucccugacgaagcuuaagggcccucugaccaugauggccagccacuauaagcagcacugcccuccuaccccugaaacgucgugugcuacccagaucaucaccuucgagagcuucaaggagaaucugaaggacuuccugcucguuauuccguucgauuguugggagccugugcaggag

SEQ ID NO: 25 - RNA, 2238 nt, Artificial Sequence
  source: /note=“Description of Artificial Sequence: Synthetic polynucleotide”
  modified_base (2)..(2): N1-methylpseudouracil
  modified_base (7)..(7): N1-methylpseudouracil
  modified_base (11)..(11): N1-methylpseudouracil
  modified_base (16)..(17): N1-methylpseudouracil
  modified_base (20)..(20): N1-methylpseudouracil
  modified_base (26)..(26): N1-methylpseudouracil
  modified_base (29)..(29): N1-methylpseudouracil
  modified_base (31)..(32): N1-methylpseudouracil
  modified_base (35)..(35): N1-methylpseudouracil
  modified_base (37)..(38): N1-methylpseudouracil
  modified_base (49)..(49): N1-methylpseudouracil
  modified_base (62)..(62): N1-methylpseudouracil
  modified_base (64)..(65): N1-methylpseudouracil
  modified_base (92)..(92): N1-methylpseudouracil
  modified_base (103)..(104): N1-methylpseudouracil
  modified_base (113)..(113): N1-methylpseudouracil
  modified_base (127)..(128): N1-methylpseudouracil
  modified_base (137)..(137): N1-methylpseudouracil
  modified_base (140)..(140): N1-methylpseudouracil
  modified_base (143)..(143): N1-methylpseudouracil
  modified_base (146)..(146): N1-methylpseudouracil
  modified_base (151)..(152): N1-methylpseudouracil
  modified_base (160)..(160): N1-methylpseudouracil
  modified_base (164)..(164): N1-methylpseudouracil
  modified_base (172)..(172): N1-methylpseudouracil
  modified_base (177)..(179): N1-methylpseudouracil
  modified_base (191)..(191): N1-methylpseudouracil
  modified_base (197)..(197): N1-methylpseudouracil
  modified_base (200)..(200): N1-methylpseudouracil
  modified_base (209)..(209): N1-methylpseudouracil
  modified_base (217)..(218): N1-methylpseudouracil
  modified_base (229)..(229): N1-methylpseudouracil
  modified_base (233)..(233): N1-methylpseudouracil
  modified_base (256)..(256): N1-methylpseudouracil
  modified_base (269)..(269): N1-methylpseudouracil
  modified_base (278)..(278): N1-methylpseudouracil
  modified_base (280)..(281): N1-methylpseudouracil
  modified_base (293)..(293): N1-methylpseudouracil
  modified_base (295)..(295): N1-methylpseudouracil
  modified_base (302)..(302): N1-methylpseudouracil
  modified_base (311)..(311): N1-methylpseudouracil
  modified_base (321)..(322): N1-methylpseudouracil
  modified_base (332)..(332): N1-methylpseudouracil
  modified_base (340)..(340): N1-methylpseudouracil
  modified_base (343)..(343): N1-methylpseudouracil
  modified_base (373)..(373): N1-methylpseudouracil
  modified_base (376)..(377): N1-methylpseudouracil
  modified_base (380)..(380): N1-methylpseudouracil
  modified_base (402)..(402): N1-methylpseudouracil
  modified_base (407)..(407): N1-methylpseudouracil
  modified_base (411)..(411): N1-methylpseudouracil
  modified_base (416)..(416): N1-methylpseudouracil
  modified_base (419)..(419): N1-methylpseudouracil
  modified_base (426)..(426): N1-methylpseudouracil
  modified_base (431)..(431): N1-methylpseudouracil
  modified_base (437)..(437): N1-methylpseudouracil
  modified_base (440)..(440): N1-methylpseudouracil
  modified_base (442)..(442): N1-methylpseudouracil
  modified_base (444)..(444): N1-methylpseudouracil
  modified_base (451)..(452): N1-methylpseudouracil
  modified_base (472)..(473): N1-methylpseudouracil
  modified_base (476)..(476): N1-methylpseudouracil
  modified_base (484)..(484): N1-methylpseudouracil
  modified_base (488)..(488): N1-methylpseudouracil
  modified_base (490)..(490): N1-methylpseudouracil
  modified_base (497)..(497): N1-methylpseudouracil
  modified_base (513)..(514): N1-methylpseudouracil
  modified_base (517)..(518): N1-methylpseudouracil
  modified_base (520)..(520): N1-methylpseudouracil
  modified_base (528)..(528): N1-methylpseudouracil
  modified_base (532)..(533): N1-methylpseudouracil
  modified_base (536)..(536): N1-methylpseudouracil
  modified_base (538)..(539): N1-methylpseudouracil
  modified_base (541)..(542): N1-methylpseudouracil
  modified_base (553)..(553): N1-methylpseudouracil
  modified_base (565)..(566): N1-methylpseudouracil
  modified_base (574)..(574): N1-methylpseudouracil
  modified_base (577)..(577): N1-methylpseudouracil
  modified_base (591)..(591): N1-methylpseudouracil
  modified_base (601)..(601): N1-methylpseudouracil
  modified_base (605)..(605): N1-methylpseudouracil
  modified_base (608)..(608): N1-methylpseudouracil
  modified_base (612)..(612): N1-methylpseudouracil
  modified_base (617)..(617): N1-methylpseudouracil
  modified_base (626)..(626): N1-methylpseudouracil
  modified_base (646)..(646): N1-methylpseudouracil
  modified_base (654)..(654): N1-methylpseudouracil
  modified_base (665)..(665): N1-methylpseudouracil
  modified_base (670)..(670): N1-methylpseudouracil
  modified_base (680)..(680): N1-methylpseudouracil
  modified_base (688)..(689): N1-methylpseudouracil
  modified_base (702)..(704): N1-methylpseudouracil
  modified_base (712)..(712): N1-methylpseudouracil
  modified_base (719)..(719): N1-methylpseudouracil
  modified_base (723)..(723): N1-methylpseudouracil
  modified_base (727)..(728): N1-methylpseudouracil
  modified_base (739)..(740): N1-methylpseudouracil
  modified_base (744)..(744): N1-methylpseudouracil
  modified_base (754)..(755): N1-methylpseudouracil
  modified_base (759)..(759): N1-methylpseudouracil
  modified_base (764)..(764): N1-methylpseudouracil
  modified_base (773)..(773): N1-methylpseudouracil
  modified_base (776)..(776): N1-methylpseudouracil
  modified_base (780)..(780): N1-methylpseudouracil
  modified_base (785)..(785): N1-methylpseudouracil
  modified_base (794)..(794): N1-methylpseudouracil
  modified_base (798)..(798): N1-methylpseudouracil
  modified_base (805)..(805): N1-methylpseudouracil
  modified_base (808)..(808): N1-methylpseudouracil
  modified_base (810)..(810): N1-methylpseudouracil
  modified_base (821)..(821): N1-methylpseudouracil
  modified_base (824)..(824): N1-methylpseudouracil
  modified_base (829)..(829): N1-methylpseudouracil
  modified_base (851)..(851): N1-methylpseudouracil
  modified_base (859)..(859): N1-methylpseudouracil
  modified_base (863)..(863): N1-methylpseudouracil
  modified_base (865)..(865): N1-methylpseudouracil
  modified_base (884)..(884): N1-methylpseudouracil
  modified_base (896)..(896): N1-methylpseudouracil
  modified_base (904)..(904): N1-methylpseudouracil
  modified_base (906)..(907): N1-methylpseudouracil
  modified_base (909)..(909): N1-methylpseudouracil
  modified_base (918)..(918): N1-methylpseudouracil
  modified_base (920)..(920): N1-methylpseudouracil
  modified_base (922)..(923): N1-methylpseudouracil
  modified_base (933)..(933): N1-methylpseudouracil
  modified_base (937)..(937): N1-methylpseudouracil
  modified_base (941)..(942): N1-methylpseudouracil
  modified_base (950)..(950): N1-methylpseudouracil
  modified_base (965)..(965): N1-methylpseudouracil
  modified_base (969)..(969): N1-methylpseudouracil
  modified_base (975)..(975): N1-methylpseudouracil
  modified_base (977)..(977): N1-methylpseudouracil
  modified_base (985)..(986): N1-methylpseudouracil
  modified_base (997)..(998): N1-methylpseudouracil
  modified_base (1001)..(1001): N1-methylpseudouracil
  modified_base (1016)..(1016): N1-methylpseudouracil
  modified_base (1018)..(1018): N1-methylpseudouracil
  modified_base (1027)..(1027): N1-methylpseudouracil
  modified_base (1046)..(1049): N1-methylpseudouracil
  modified_base (1052)..(1052): N1-methylpseudouracil
  modified_base (1058)..(1058): N1-methylpseudouracil
  modified_base (1060)..(1061): N1-methylpseudouracil
  modified_base (1064)..(1064): N1-methylpseudouracil
  modified_base (1066)..(1066): N1-methylpseudouracil
  modified_base (1072)..(1072): N1-methylpseudouracil
  modified_base (1086)..(1086): N1-methylpseudouracil
  modified_base (1093)..(1093): N1-methylpseudouracil
  modified_base (1100)..(1100): N1-methylpseudouracil
  modified_base (1103)..(1103): N1-methylpseudouracil
  modified_base (1106)..(1106): N1-methylpseudouracil
  modified_base (1109)..(1109): N1-methylpseudouracil
  modified_base (1112)..(1112): N1-methylpseudouracil
  modified_base (1118)..(1118): N1-methylpseudouracil
  modified_base (1122)..(1122): N1-methylpseudouracil
  modified_base (1129)..(1129): N1-methylpseudouracil
  modified_base (1137)..(1137): N1-methylpseudouracil
  modified_base (1142)..(1142): N1-methylpseudouracil
  modified_base (1150)..(1150): N1-methylpseudouracil
  modified_base (1153)..(1153): N1-methylpseudouracil
  modified_base (1170)..(1170): N1-methylpseudouracil
  modified_base (1177)..(1177): N1-methylpseudouracil
  modified_base (1179)..(1180): N1-methylpseudouracil
  modified_base (1190)..(1190): N1-methylpseudouracil
  modified_base (1192)..(1193): N1-methylpseudouracil
  modified_base (1201)..(1202): N1-methylpseudouracil
  modified_base (1209)..(1209): N1-methylpseudouracil
  modified_base (1211)..(1211): N1-methylpseudouracil
  modified_base (1214)..(1214): N1-methylpseudouracil
  modified_base (1224)..(1224): N1-methylpseudouracil
  modified_base (1232)..(1232): N1-methylpseudouracil
  modified_base (1235)..(1235): N1-methylpseudouracil
  modified_base (1246)..(1246): N1-methylpseudouracil
  modified_base (1248)..(1248): N1-methylpseudouracil
  modified_base (1253)..(1253): N1-methylpseudouracil
  modified_base (1255)..(1256): N1-methylpseudouracil
  modified_base (1265)..(1265): N1-methylpseudouracil
  modified_base (1273)..(1273): N1-methylpseudouracil
  modified_base (1279)..(1280): N1-methylpseudouracil
  modified_base (1292)..(1292): N1-methylpseudouracil
  modified_base (1294)..(1295): N1-methylpseudouracil
  modified_base (1298)..(1298): N1-methylpseudouracil
  modified_base (1303)..(1303): N1-methylpseudouracil
  modified_base (1316)..(1316): N1-methylpseudouracil
  modified_base (1320)..(1320): N1-methylpseudouracil
  modified_base (1325)..(1325): N1-methylpseudouracil
  modified_base (1329)..(1329): N1-methylpseudouracil
  modified_base (1335)..(1335): N1-methylpseudouracil
  modified_base (1340)..(1340): N1-methylpseudouracil
  modified_base (1343)..(1343): N1-methylpseudouracil
  modified_base (1349)..(1350): N1-methylpseudouracil
  modified_base (1353)..(1353): N1-methylpseudouracil
  modified_base (1361)..(1361): N1-methylpseudouracil
  modified_base (1370)..(1370): N1-methylpseudouracil
  modified_base (1381)..(1381): N1-methylpseudouracil
  modified_base (1384)..(1384): N1-methylpseudouracil
  modified_base (1386)..(1386): N1-methylpseudouracil
  modified_base (1401)..(1401): N1-methylpseudouracil
  modified_base (1409)..(1409): N1-methylpseudouracil
  modified_base (1413)..(1414): N1-methylpseudouracil
  modified_base (1426)..(1426): N1-methylpseudouracil
  modified_base (1430)..(1430): N1-methylpseudouracil
  modified_base (1432)..(1432): N1-methylpseudouracil
  modified_base (1436)..(1437): N1-methylpseudouracil
  modified_base (1439)..(1439): N1-methylpseudouracil
  modified_base (1442)..(1442): N1-methylpseudouracil
  modified_base (1451)..(1451): N1-methylpseudouracil
  modified_base (1453)..(1453): N1-methylpseudouracil
  modified_base (1457)..(1457): N1-methylpseudouracil
  modified_base (1460)..(1460): N1-methylpseudouracil
  modified_base (1476)..(1476): N1-methylpseudouracil
  modified_base (1478)..(1478): N1-methylpseudouracil
  modified_base (1490)..(1490): N1-methylpseudouracil
  modified_base (1498)..(1498): N1-methylpseudouracil
  modified_base (1500)..(1501): N1-methylpseudouracil
  modified_base (1513)..(1514): N1-methylpseudouracil
  modified_base (1517)..(1517): N1-methylpseudouracil
  modified_base (1521)..(1521): N1-methylpseudouracil
  modified_base (1530)..(1531): N1-methylpseudouracil
  modified_base (1534)..(1535): N1-methylpseudouracil
  modified_base (1544)..(1544): N1-methylpseudouracil
  modified_base (1550)..(1551): N1-methylpseudouracil
  modified_base (1560)..(1561): N1-methylpseudouracil
  modified_base (1565)..(1565): N1-methylpseudouracil
  modified_base (1569)..(1569): N1-methylpseudouracil
  modified_base (1576)..(1577): N1-methylpseudouracil
  modified_base (1590)..(1592): N1-methylpseudouracil
  modified_base (1597)..(1598): N1-methylpseudouracil
  modified_base (1610)..(1610): N1-methylpseudouracil
  modified_base (1612)..(1612): N1-methylpseudouracil
  modified_base (1614)..(1614): N1-methylpseudouracil
  modified_base (1617)..(1617): N1-methylpseudouracil
  modified_base (1619)..(1619): N1-methylpseudouracil
  modified_base (1640)..(1640): N1-methylpseudouracil
  modified_base (1658)..(1658): N1-methylpseudouracil
  modified_base (1661)..(1661): N1-methylpseudouracil
  modified_base (1666)..(1667): N1-methylpseudouracil
  modified_base (1670)..(1670): N1-methylpseudouracil
  modified_base (1703)..(1704): N1-methylpseudouracil
  modified_base (1712)..(1712): N1-methylpseudouracil
  modified_base (1715)..(1715): N1-methylpseudouracil
  modified_base (1723)..(1724): N1-methylpseudouracil
  modified_base (1732)..(1733): N1-methylpseudouracil
  modified_base (1736)..(1736): N1-methylpseudouracil
  modified_base (1744)..(1744): N1-methylpseudouracil
  modified_base (1746)..(1747): N1-methylpseudouracil
  modified_base (1749)..(1749): N1-methylpseudouracil
  modified_base (1770)..(1771): N1-methylpseudouracil
  modified_base (1774)..(1775): N1-methylpseudouracil
  modified_base (1795)..(1796): N1-methylpseudouracil
  modified_base (1799)..(1799): N1-methylpseudouracil
  modified_base (1807)..(1807): N1-methylpseudouracil
  modified_base (1809)..(1809): N1-methylpseudouracil
  modified_base (1815)..(1815): N1-methylpseudouracil
  modified_base (1819)..(1820): N1-methylpseudouracil
  modified_base (1826)..(1826): N1-methylpseudouracil
  modified_base (1830)..(1830): N1-methylpseudouracil
  modified_base (1845)..(1845): N1-methylpseudouracil
  modified_base (1855)..(1855): N1-methylpseudouracil
  modified_base (1874)..(1874): N1-methylpseudouracil
  modified_base (1880)..(1880): N1-methylpseudouracil
  modified_base (1889)..(1889): N1-methylpseudouracil
  modified_base (1892)..(1892): N1-methylpseudouracil
  modified_base (1895)..(1895): N1-methylpseudouracil
  modified_base (1900)..(1901): N1-methylpseudouracil
  modified_base (1907)..(1907): N1-methylpseudouracil
  modified_base (1910)..(1910): N1-methylpseudouracil
  modified_base (1913)..(1913): N1-methylpseudouracil
  modified_base (1922)..(1922): N1-methylpseudouracil
  modified_base (1930)..(1930): N1-methylpseudouracil
  modified_base (1946)..(1946): N1-methylpseudouracil
  modified_base (1955)..(1955): N1-methylpseudouracil
  modified_base (1958)..(1958): N1-methylpseudouracil
  modified_base (1963)..(1964): N1-methylpseudouracil
  modified_base (1969)..(1970): N1-methylpseudouracil
  modified_base (1972)..(1972): N1-methylpseudouracil
  modified_base (1976)..(1976): N1-methylpseudouracil
  modified_base (1980)..(1980): N1-methylpseudouracil
  modified_base (1992)..(1992): N1-methylpseudouracil
  modified_base (1997)..(1997): N1-methylpseudouracil
  modified_base (2006)..(2007): N1-methylpseudouracil
  modified_base (2011)..(2011): N1-methylpseudouracil
  modified_base (2015)..(2015): N1-methylpseudouracil
  modified_base (2027)..(2028): N1-methylpseudouracil
  modified_base (2036)..(2036): N1-methylpseudouracil
  modified_base (2048)..(2048): N1-methylpseudouracil
  modified_base (2054)..(2054): N1-methylpseudouracil
  modified_base (2061)..(2061): N1-methylpseudouracil
  modified_base (2064)..(2064): N1-methylpseudouracil
  modified_base (2070)..(2072): N1-methylpseudouracil
  modified_base (2078)..(2078): N1-methylpseudouracil
  modified_base (2085)..(2085): N1-methylpseudouracil
  modified_base (2093)..(2094): N1-methylpseudouracil
  modified_base (2096)..(2097): N1-methylpseudouracil
  modified_base (2105)..(2105): N1-methylpseudouracil
  modified_base (2111)..(2111): N1-methylpseudouracil
  modified_base (2114)..(2114): N1-methylpseudouracil
  modified_base (2117)..(2117): N1-methylpseudouracil
  modified_base (2120)..(2120): N1-methylpseudouracil
  modified_base (2125)..(2126): N1-methylpseudouracil
  modified_base (2134)..(2134): N1-methylpseudouracil
  modified_base (2146)..(2147): N1-methylpseudouracil
  modified_base (2150)..(2150): N1-methylpseudouracil
  modified_base (2152)..(2152): N1-methylpseudouracil
  modified_base (2158)..(2158): N1-methylpseudouracil
  modified_base (2180)..(2180): N1-methylpseudouracil
  modified_base (2183)..(2183): N1-methylpseudouracil
  modified_base (2188)..(2189): N1-methylpseudouracil
  modified_base (2192)..(2192): N1-methylpseudouracil
  modified_base (2200)..(2200): N1-methylpseudouracil
  modified_base (2204)..(2204): N1-methylpseudouracil
  modified_base (2209)..(2210): N1-methylpseudouracil
  modified_base (2212)..(2212): N1-methylpseudouracil
  modified_base (2222)..(2222): N1-methylpseudouracil
  modified_base (2225)..(2225): N1-methylpseudouracil
  modified_base (2227)..(2227): N1-methylpseudouracil
  modified_base (2229)..(2229): N1-methylpseudouracil
  modified_base (2232)..(2232): N1-methylpseudouracil
  modified_base (2234)..(2234): N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 26 - RNA, 57 nt, Artificial Sequence
  source: /note=“Description of Artificial Sequence: Synthetic oligonucleotide”
  modified_base (7)..(7): N1-methylpseudouracil
  modified_base (25)..(25): N1-methylpseudouracil
  modified_base (35)..(35): N1-methylpseudouracil
  modified_base (37)..(37): N1-methylpseudouracil
gggaaanaagagagaaaagaagagnaagaagaaananaagaccccggcgccgccacc

SEQ ID NO: 27 - RNA, 119 nt, Artificial Sequence
  source: /note=“Description of Artificial Sequence: Synthetic polynucleotide”
  modified_base (1)..(1): N1-methylpseudouracil
  modified_base (4)..(4): N1-methylpseudouracil
  modified_base (7)..(7): N1-methylpseudouracil
  modified_base (12)..(12): N1-methylpseudouracil
  modified_base (19)..(19): N1-methylpseudouracil
  modified_base (23)..(23): N1-methylpseudouracil
  modified_base (28)..(28): N1-methylpseudouracil
  modified_base (32)..(33): N1-methylpseudouracil
  modified_base (35)..(36): N1-methylpseudouracil
  modified_base (42)..(43): N1-methylpseudouracil
  modified_base (49)..(49): N1-methylpseudouracil
  modified_base (62)..(62): N1-methylpseudouracil
  modified_base (65)..(65): N1-methylpseudouracil
  modified_base (70)..(71): N1-methylpseudouracil
  modified_base (74)..(74): N1-methylpseudouracil
  modified_base (82)..(82): N1-methylpseudouracil
  modified_base (90)..(90): N1-methylpseudouracil
  modified_base (93)..(93): N1-methylpseudouracil
  modified_base (95)..(97): N1-methylpseudouracil
  modified_base (101)..(101): N1-methylpseudouracil
  modified_base (106)..(106): N1-methylpseudouracil
  modified_base (108)..(108): N1-methylpseudouracil
  modified_base (112)..(112): N1-methylpseudouracil
nganaanaggcnggagccncggnggccnagcnncnngccccnngggccnccccccagccccnccnccccnnccngcacccgnacccccgnggncnnngaanaaagncngagngggcggc

SEQ ID NO: 28 - RNA, 2514 nt, Artificial Sequence
  source: /note=“Description of Artificial Sequence: Synthetic polynucleotide”
  modified_base (7)..(7): N1-methylpseudouracil
  modified_base (25)..(25): N1-methylpseudouracil
  modified_base (35)..(35): N1-methylpseudouracil
  modified_base (37)..(37): N1-methylpseudouracil
  modified_base (59)..(59): N1-methylpseudouracil
  modified_base (64)..(64): N1-methylpseudouracil
  modified_base (68)..(68): N1-methylpseudouracil
  modified_base (73)..(74): N1-methylpseudouracil
  modified_base (77)..(77): N1-methylpseudouracil
  modified_base (83)..(83): N1-methylpseudouracil
  modified_base (86)..(86): N1-methylpseudouracil
  modified_base (88)..(89): N1-methylpseudouracil
  modified_base (92)..(92): N1-methylpseudouracil
  modified_base (94)..(95): N1-methylpseudouracil
  modified_base (106)..(106): N1-methylpseudouracil
  modified_base (119)..(119): N1-methylpseudouracil
  modified_base (121)..(122): N1-methylpseudouracil
  modified_base (149)..(149): N1-methylpseudouracil
  modified_base (160)..(161): N1-methylpseudouracil
  modified_base (170)..(170): N1-methylpseudouracil
  modified_base (184)..(185): N1-methylpseudouracil
  modified_base (194)..(194): N1-methylpseudouracil
  modified_base (197)..(197): N1-methylpseudouracil
  modified_base (200)..(200): N1-methylpseudouracil
  modified_base (203)..(203): N1-methylpseudouracil
  modified_base (208)..(209): N1-methylpseudouracil
  modified_base (217)..(217): N1-methylpseudouracil
  modified_base (221)..(221): N1-methylpseudouracil
  modified_base (229)..(229): N1-methylpseudouracil
  modified_base (234)..(236): N1-methylpseudouracil
  modified_base (248)..(248): N1-methylpseudouracil
  modified_base (254)..(254): N1-methylpseudouracil
  modified_base (257)..(257): N1-methylpseudouracil
  modified_base (266)..(266): N1-methylpseudouracil
  modified_base (274)..(275): N1-methylpseudouracil
  modified_base (286)..(286): N1-methylpseudouracil
  modified_base (290)..(290): N1-methylpseudouracil
  modified_base (313)..(313): N1-methylpseudouracil
  modified_base (326)..(326): N1-methylpseudouracil
  modified_base (335)..(335): N1-methylpseudouracil
  modified_base (337)..(338): N1-methylpseudouracil
  modified_base (350)..(350): N1-methylpseudouracil
  modified_base (352)..(352): N1-methylpseudouracil
  modified_base (359)..(359): N1-methylpseudouracil
  modified_base (368)..(368): N1-methylpseudouracil
  modified_base (378)..(379): N1-methylpseudouracil
  modified_base (389)..(389): N1-methylpseudouracil
  modified_base (397)..(397): N1-methylpseudouracil
  modified_base (400)..(400): N1-methylpseudouracil
  modified_base (430)..(430): N1-methylpseudouracil
  modified_base (433)..(434): N1-methylpseudouracil
  modified_base (437)..(437): N1-methylpseudouracil
  modified_base (459)..(459): N1-methylpseudouracil
  modified_base (464)..(464): N1-methylpseudouracil
  modified_base (468)..(468): N1-methylpseudouracil
  modified_base (473)..(473): N1-methylpseudouracil
  modified_base (476)..(476): N1-methylpseudouracil
  modified_base (483)..(483): N1-methylpseudouracil
  modified_base (488)..(488): N1-methylpseudouracil
  modified_base (494)..(494): N1-methylpseudouracil
  modified_base (497)..(497): N1-methylpseudouracil
  modified_base (499)..(499): N1-methylpseudouracil
  modified_base (501)..(501): N1-methylpseudouracil
  modified_base (508)..(509): N1-methylpseudouracil
  modified_base (529)..(530): N1-methylpseudouracil
  modified_base (533)..(533): N1-methylpseudouracil
  modified_base (541)..(541): N1-methylpseudouracil
  modified_base (545)..(545): N1-methylpseudouracil
  modified_base (547)..(547): N1-methylpseudouracil
  modified_base (554)..(554): N1-methylpseudouracil
  modified_base (570)..(571): N1-methylpseudouracil
  modified_base (574)..(575): N1-methylpseudouracil
  modified_base (577)..(577): N1-methylpseudouracil
  modified_base (585)..(585): N1-methylpseudouracil
  modified_base (589)..(590): N1-methylpseudouracil
  modified_base (593)..(593): N1-methylpseudouracil
  modified_base (595)..(596): N1-methylpseudouracil
  modified_base (598)..(599): N1-methylpseudouracil
  modified_base (610)..(610): N1-methylpseudouracil
  modified_base (622)..(623): N1-methylpseudouracil
  modified_base (631)..(631): N1-methylpseudouracil
  modified_base (634)..(634): N1-methylpseudouracil
  modified_base (648)..(648): N1-methylpseudouracil
  modified_base (658)..(658): N1-methylpseudouracil
  modified_base (662)..(662): N1-methylpseudouracil
  modified_base (665)..(665): N1-methylpseudouracil
  modified_base (669)..(669): N1-methylpseudouracil
  modified_base (674)..(674): N1-methylpseudouracil
  modified_base (683)..(683): N1-methylpseudouracil
  modified_base (703)..(703): N1-methylpseudouracil
  modified_base (711)..(711): N1-methylpseudouracil
  modified_base (722)..(722): N1-methylpseudouracil
  modified_base (727)..(727): N1-methylpseudouracil
  modified_base (737)..(737): N1-methylpseudouracil
  modified_base (745)..(746): N1-methylpseudouracil
  modified_base (759)..(761): N1-methylpseudouracil
  modified_base (769)..(769): N1-methylpseudouracil
  modified_base (776)..(776): N1-methylpseudouracil
  modified_base (780)..(780): N1-methylpseudouracil
  modified_base (784)..(785): N1-methylpseudouracil
  modified_base (796)..(797): N1-methylpseudouracil
  modified_base (801)..(801): N1-methylpseudouracil
  modified_base (811)..(812): N1-methylpseudouracil
  modified_base (816)..(816): N1-methylpseudouracil
  modified_base (821)..(821): N1-methylpseudouracil
  modified_base (830)..(830): N1-methylpseudouracil
  modified_base (833)..(833): N1-methylpseudouracil
  modified_base (837)..(837): N1-methylpseudouracil
  modified_base (842)..(842): N1-methylpseudouracil
  modified_base (851)..(851): N1-methylpseudouracil
  modified_base (855)..(855): N1-methylpseudouracil
  modified_base (862)..(862): N1-methylpseudouracil
  modified_base (865)..(865): N1-methylpseudouracil
  modified_base (867)..(867): N1-methylpseudouracil
  modified_base (878)..(878): N1-methylpseudouracil
  modified_base (881)..(881): N1-methylpseudouracil
  modified_base (886)..(886): N1-methylpseudouracil
  modified_base (908)..(908): N1-methylpseudouracil
  modified_base (916)..(916): N1-methylpseudouracil
  modified_base (920)..(920): N1-methylpseudouracil
  modified_base (922)..(922): N1-methylpseudouracil
  modified_base (941)..(941): N1-methylpseudouracil
  modified_base (953)..(953): N1-methylpseudouracil
  modified_base (961)..(961): N1-methylpseudouracil
  modified_base (963)..(964): N1-methylpseudouracil
  modified_base (966)..(966): N1-methylpseudouracil
  modified_base (975)..(975): N1-methylpseudouracil
  modified_base (977)..(977): N1-methylpseudouracil
  modified_base (979)..(980): N1-methylpseudouracil
  modified_base (990)..(990): N1-methylpseudouracil
  modified_base (994)..(994): N1-methylpseudouracil
  modified_base (998)..(999): N1-methylpseudouracil
  modified_base (1007)..(1007): N1-methylpseudouracil
  modified_base (1022)..(1022): N1-methylpseudouracil
  modified_base (1026)..(1026): N1-methylpseudouracil
  modified_base (1032)..(1032): N1-methylpseudouracil
  modified_base (1034)..(1034): N1-methylpseudouracil
  modified_base (1042)..(1043): N1-methylpseudouracil
  modified_base (1054)..(1055): N1-methylpseudouracil
  modified_base (1058)..(1058): N1-methylpseudouracil
  modified_base (1073)..(1073): N1-methylpseudouracil
  modified_base (1075)..(1075): N1-methylpseudouracil
  modified_base (1084)..(1084): N1-methylpseudouracil
  modified_base (1103)..(1106): N1-methylpseudouracil
  modified_base (1109)..(1109): N1-methylpseudouracil
  modified_base (1115)..(1115): N1-methylpseudouracil
  modified_base (1117)..(1118): N1-methylpseudouracil
  modified_base (1121)..(1121): N1-methylpseudouracil
  modified_base (1123)..(1123): N1-methylpseudouracil
  modified_base (1129)..(1129): N1-methylpseudouracil
  modified_base (1143)..(1143): N1-methylpseudouracil
  modified_base (1150)..(1150): N1-methylpseudouracil
  modified_base (1157)..(1157): N1-methylpseudouracil
  modified_base (1160)..(1160): N1-methylpseudouracil
  modified_base (1163)..(1163): N1-methylpseudouracil
  modified_base (1166)..(1166): N1-methylpseudouracil
  modified_base (1169)..(1169): N1-methylpseudouracil
  modified_base (1175)..(1175): N1-methylpseudouracil
  modified_base (1179)..(1179): N1-methylpseudouracil
  modified_base (1186)..(1186): N1-methylpseudouracil
  modified_base (1194)..(1194): N1-methylpseudouracil
  modified_base (1199)..(1199): N1-methylpseudouracil
  modified_base (1207)..(1207): N1-methylpseudouracil
  modified_base (1210)..(1210): N1-methylpseudouracil
  modified_base (1227)..(1227): N1-methylpseudouracil
  modified_base (1234)..(1234): N1-methylpseudouracil
  modified_base (1236)..(1237): N1-methylpseudouracil
  modified_base (1247)..(1247): N1-methylpseudouracil
  modified_base (1249)..(1250): N1-methylpseudouracil
  modified_base (1258)..(1259): N1-methylpseudouracil
  modified_base (1266)..(1266): N1-methylpseudouracil
  modified_base (1268)..(1268): N1-methylpseudouracil
  modified_base (1271)..(1271): N1-methylpseudouracil
  modified_base (1281)..(1281): N1-methylpseudouracil
  modified_base (1289)..(1289): N1-methylpseudouracil
  modified_base (1292)..(1292): N1-methylpseudouracil
  modified_base (1303)..(1303): N1-methylpseudouracil
  modified_base (1305)..(1305): N1-methylpseudouracil
  modified_base (1310)..(1310): N1-methylpseudouracil
  modified_base (1312)..(1313): N1-methylpseudouracil
  modified_base (1322)..(1322): N1-methylpseudouracil
  modified_base (1330)..(1330): N1-methylpseudouracil
  modified_base (1336)..(1337): N1-methylpseudouracil
  modified_base (1349)..(1349): N1-methylpseudouracil
  modified_base (1351)..(1352): N1-methylpseudouracil
  modified_base (1355)..(1355): N1-methylpseudouracil
  modified_base (1360)..(1360): N1-methylpseudouracil
  modified_base (1373)..(1373): N1-methylpseudouracil
  modified_base (1377)..(1377): N1-methylpseudouracil
  modified_base (1382)..(1382): N1-methylpseudouracil
  modified_base (1386)..(1386): N1-methylpseudouracil
  modified_base (1392)..(1392): N1-methylpseudouracil
  modified_base (1397)..(1397): N1-methylpseudouracil
  modified_base (1400)..(1400): N1-methylpseudouracil
  modified_base (1406)..(1407): N1-methylpseudouracil
  modified_base (1410)..(1410): N1-methylpseudouracil
  modified_base (1418)..(1418): N1-methylpseudouracil
  modified_base (1427)..(1427): N1-methylpseudouracil
  modified_base (1438)..(1438): N1-methylpseudouracil
  modified_base (1441)..(1441): N1-methylpseudouracil
  modified_base (1443)..(1443): N1-methylpseudouracil
  modified_base (1458)..(1458): N1-methylpseudouracil
  modified_base (1466)..(1466): N1-methylpseudouracil
  modified_base (1470)..(1471): N1-methylpseudouracil
  modified_base (1483)..(1483): N1-methylpseudouracil
  modified_base (1487)..(1487): N1-methylpseudouracil
  modified_base (1489)..(1489): N1-methylpseudouracil
  modified_base (1493)..(1494): N1-methylpseudouracil
  modified_base (1496)..(1496): N1-methylpseudouracil
  modified_base (1499)..(1499): N1-methylpseudouracil
  modified_base (1508)..(1508): N1-methylpseudouracil
  modified_base (1510)..(1510): N1-methylpseudouracil
  modified_base (1514)..(1514): N1-methylpseudouracil
  modified_base (1517)..(1517): N1-methylpseudouracil
  modified_base (1533)..(1533): N1-methylpseudouracil
  modified_base (1535)..(1535): N1-methylpseudouracil
  modified_base (1547)..(1547): N1-methylpseudouracil
  modified_base (1555)..(1555): N1-methylpseudouracil
  modified_base (1557)..(1558): N1-methylpseudouracil
  modified_base (1570)..(1571): N1-methylpseudouracil
  modified_base (1574)..(1574): N1-methylpseudouracil
  modified_base (1578)..(1578): N1-methylpseudouracil
  modified_base (1587)..(1588): N1-methylpseudouracil
  modified_base (1591)..(1592): N1-methylpseudouracil
  modified_base (1601)..(1601): N1-methylpseudouracil
  modified_base (1607)..(1608): N1-methylpseudouracil
  modified_base (1617)..(1618): N1-methylpseudouracil
  modified_base (1622)..(1622): N1-methylpseudouracil
  modified_base (1626)..(1626): N1-methylpseudouracil
  modified_base (1633)..(1634): N1-methylpseudouracil
  modified_base (1647)..(1649): N1-methylpseudouracil
  modified_base (1654)..(1655): N1-methylpseudouracil
  modified_base (1667)..(1667): N1-methylpseudouracil
  modified_base (1669)..(1669): N1-methylpseudouracil
  modified_base (1671)..(1671): N1-methylpseudouracil
  modified_base (1674)..(1674): N1-methylpseudouracil
  modified_base (1676)..(1676): N1-methylpseudouracil
  modified_base (1697)..(1697): N1-methylpseudouracil
  modified_base (1715)..(1715): N1-methylpseudouracil
  modified_base (1718)..(1718): N1-methylpseudouracil
  modified_base (1723)..(1724): N1-methylpseudouracil
  modified_base (1727)..(1727): N1-methylpseudouracil
  modified_base (1760)..(1761): N1-methylpseudouracil
  modified_base (1769)..(1769): N1-methylpseudouracil
  modified_base (1772)..(1772): N1-methylpseudouracil
  modified_base (1780)..(1781): N1-methylpseudouracil
  modified_base (1789)..(1790): N1-methylpseudouracil
  modified_base (1793)..(1793): N1-methylpseudouracil
  modified_base (1801)..(1801): N1-methylpseudouracil
  modified_base (1803)..(1804): N1-methylpseudouracil
  modified_base (1806)..(1806): N1-methylpseudouracil
  modified_base (1827)..(1828): N1-methylpseudouracil
  modified_base (1831)..(1832): N1-methylpseudouracil
  modified_base (1852)..(1853): N1-methylpseudouracil
  modified_base (1856)..(1856): N1-methylpseudouracil
  modified_base (1864)..(1864): N1-methylpseudouracil
  modified_base (1866)..(1866): N1-methylpseudouracil
  modified_base (1872)..(1872): N1-methylpseudouracil
  modified_base (1876)..(1877): N1-methylpseudouracil
  modified_base (1883)..(1883): N1-methylpseudouracil
  modified_base (1887)..(1887): N1-methylpseudouracil
  modified_base (1902)..(1902): N1-methylpseudouracil
  modified_base (1912)..(1912): N1-methylpseudouracil
  modified_base (1931)..(1931): N1-methylpseudouracil
  modified_base (1937)..(1937): N1-methylpseudouracil
  modified_base (1946)..(1946): N1-methylpseudouracil
  modified_base (1949)..(1949): N1-methylpseudouracil
  modified_base (1952)..(1952): N1-methylpseudouracil
  modified_base (1957)..(1958): N1-methylpseudouracil
  modified_base (1964)..(1964): N1-methylpseudouracil
  modified_base (1967)..(1967): N1-methylpseudouracil
  modified_base (1970)..(1970): N1-methylpseudouracil
  modified_base (1979)..(1979): N1-methylpseudouracil
  modified_base (1987)..(1987): N1-methylpseudouracil
  modified_base (2003)..(2003): N1-methylpseudouracil
  modified_base (2012)..(2012): N1-methylpseudouracil
  modified_base (2015)..(2015): N1-methylpseudouracil
  modified_base (2020)..(2021): N1-methylpseudouracil
  modified_base (2026)..(2027): N1-methylpseudouracil
  modified_base (2029)..(2029): N1-methylpseudouracil
  modified_base (2033)..(2033): N1-methylpseudouracil
  modified_base (2037)..(2037): N1-methylpseudouracil
  modified_base (2049)..(2049): N1-methylpseudouracil
  modified_base (2054)..(2054): N1-methylpseudouracil
  modified_base (2063)..(2064): N1-methylpseudouracil
  modified_base (2068)..(2068): N1-methylpseudouracil
  modified_base (2072)..(2072): N1-methylpseudouracil
  modified_base (2084)..(2085): N1-methylpseudouracil
  modified_base (2093)..(2093): N1-methylpseudouracil
  modified_base (2105)..(2105): N1-methylpseudouracil
  modified_base (2111)..(2111): N1-methylpseudouracil
  modified_base (2118)..(2118): N1-methylpseudouracil
  modified_base (2121)..(2121): N1-methylpseudouracil
  modified_base (2127)..(2129): N1-methylpseudouracil
  modified_base (2135)..(2135): N1-methylpseudouracil
  modified_base (2142)..(2142): N1-methylpseudouracil
  modified_base (2150)..(2151): N1-methylpseudouracil
  modified_base (2153)..(2154): N1-methylpseudouracil
  modified_base (2162)..(2162): N1-methylpseudouracil
  modified_base (2168)..(2168): N1-methylpseudouracil
  modified_base (2171)..(2171): N1-methylpseudouracil
  modified_base (2174)..(2174): N1-methylpseudouracil
  modified_base (2177)..(2177): N1-methylpseudouracil
  modified_base (2182)..(2183): N1-methylpseudouracil
  modified_base (2191)..(2191): N1-methylpseudouracil
  modified_base (2203)..(2204): N1-methylpseudouracil
  modified_base (2207)..(2207): N1-methylpseudouracil
  modified_base (2209)..(2209): N1-methylpseudouracil
  modified_base (2215)..(2215): N1-methylpseudouracil
  modified_base (2237)..(2237): N1-methylpseudouracil
  modified_base (2240)..(2240): N1-methylpseudouracil
  modified_base (2245)..(2246): N1-methylpseudouracil
  modified_base (2249)..(2249): N1-methylpseudouracil
  modified_base (2257)..(2257): N1-methylpseudouracil
  modified_base (2261)..(2261): N1-methylpseudouracil
  modified_base (2266)..(2267): N1-methylpseudouracil
  modified_base (2269)..(2269): N1-methylpseudouracil
  modified_base (2279)..(2279): N1-methylpseudouracil
  modified_base (2282)..(2282): N1-methylpseudouracil
  modified_base (2284)..(2284): N1-methylpseudouracil
  modified_base (2286)..(2286): N1-methylpseudouracil
  modified_base (2289)..(2289): N1-methylpseudouracil
  modified_base (2291)..(2291): N1-methylpseudouracil
  modified_base (2296)..(2296): N1-methylpseudouracil
  modified_base (2299)..(2299): N1-methylpseudouracil
  modified_base (2302)..(2302): N1-methylpseudouracil
  modified_base (2307)..(2307): N1-methylpseudouracil
  modified_base (2314)..(2314): N1-methylpseudouracil
  modified_base (2318)..(2318): N1-methylpseudouracil
  modified_base (2323)..(2323): N1-methylpseudouracil
  modified_base (2327)..(2328): N1-methylpseudouracil
  modified_base (2330)..(2331): N1-methylpseudouracil
  modified_base (2337)..(2338): N1-methylpseudouracil
  modified_base (2344)..(2344): N1-methylpseudouracil
  modified_base (2357)..(2357): N1-methylpseudouracil
  modified_base (2360)..(2360): N1-methylpseudouracil
  modified_base (2365)..(2366): N1-methylpseudouracil
  modified_base (2369)..(2369): N1-methylpseudouracil
  modified_base (2377)..(2377): N1-methylpseudouracil
  modified_base (2385)..(2385): N1-methylpseudouracil
  modified_base (2388)..(2388): N1-methylpseudouracil
  modified_base (2390)..(2392): N1-methylpseudouracil
  modified_base (2396)..(2396): N1-methylpseudouracil
  modified_base (2401)..(2401): N1-methylpseudouracil
  modified_base (2403)..(2403): N1-methylpseudouracil
  modified_base (2407)..(2407): N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - protein, 153 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 32 - protein, 153 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 33 - protein, 153 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 34 - protein, 153 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 35 - protein, 153 aa, Artificial Sequence
  source: /note=“Description of Artificial Sequence: Synthetic polypeptide”
MYRMQLLSCIALSLALVTNSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - RNA, 2238 nt, Artificial Sequence
  source: /note=“Description of Artificial Sequence: Synthetic polynucleotide”
  modified_base (2)..(2): N1-methylpseudouracil
  modified_base (7)..(7): N1-methylpseudouracil
  modified_base (11)..(11): N1-methylpseudouracil
  modified_base (16)..(17): N1-methylpseudouracil
  modified_base (20)..(20): N1-methylpseudouracil
  modified_base (26)..(26): N1-methylpseudouracil
  modified_base (29)..(29): N1-methylpseudouracil
  modified_base (31)..(32): N1-methylpseudouracil
  modified_base (35)..(35): N1-methylpseudouracil
  modified_base (37)..(38): N1-methylpseudouracil
  modified_base (49)..(49): N1-methylpseudouracil
  modified_base (62)..(62): N1-methylpseudouracil
  modified_base (64)..(65): N1-methylpseudouracil
  modified_base (92)..(92): N1-methylpseudouracil
  modified_base (103)..(104): N1-methylpseudouracil
  modified_base (113)..(113): N1-methylpseudouracil
  modified_base (127)..(128): N1-methylpseudouracil
  modified_base (137)..(137): N1-methylpseudouracil
  modified_base (140)..(140): N1-methylpseudouracil
  modified_base (143)..(143): N1-methylpseudouracil
  modified_base (146)..(146): N1-methylpseudouracil
  modified_base (151)..(152): N1-methylpseudouracil
  modified_base (160)..(160): N1-methylpseudouracil
  modified_base (164)..(164): N1-methylpseudouracil
  modified_base (172)..(172): N1-methylpseudouracil
  modified_base (177)..(179): N1-methylpseudouracil
  modified_base (191)..(191): N1-methylpseudouracil
  modified_base (197)..(197): N1-methylpseudouracil
  modified_base (200)..(200): N1-methylpseudouracil
  modified_base (209)..(209): N1-methylpseudouracil
  modified_base (217)..(218): N1-methylpseudouracil
  modified_base (229)..(229): N1-methylpseudouracil
  modified_base (233)..(233): N1-methylpseudouracil
  modified_base (256)..(256): N1-methylpseudouracil
  modified_base (269)..(269): N1-methylpseudouracil
  modified_base (278)..(278): N1-methylpseudouracil
  modified_base (280)..(281): N1-methylpseudouracil
  modified_base (293)..(293): N1-methylpseudouracil
  modified_base (295)..(295): N1-methylpseudouracil
  modified_base (302)..(302): N1-methylpseudouracil
  modified_base (311)..(311): N1-methylpseudouracil
  modified_base (321)..(322): N1-methylpseudouracil
  modified_base (332)..(332): N1-methylpseudouracil
  modified_base (340)..(340): N1-methylpseudouracil
  modified_base (343)..(343): N1-methylpseudouracil
  modified_base (373)..(373): N1-methylpseudouracil
  modified_base (376)..(377): N1-methylpseudouracil
  modified_base (380)..(380): N1-methylpseudouracil
  modified_base (402)..(402): N1-methylpseudouracil
  modified_base (407)..(407): N1-methylpseudouracil
  modified_base (411)..(411): N1-methylpseudouracil
  modified_base (416)..(416): N1-methylpseudouracil
  modified_base (419)..(419): N1-methylpseudouracil
  modified_base (426)..(426): N1-methylpseudouracil
  modified_base (431)..(431): N1-methylpseudouracil
  modified_base (437)..(437): N1-methylpseudouracil
  modified_base (440)..(440): N1-methylpseudouracil
  modified_base (442)..(442): N1-methylpseudouracil
  modified_base (444)..(444): N1-methylpseudouracil
  modified_base (451)..(452): N1-methylpseudouracil
  modified_base (472)..(473): N1-methylpseudouracil
  modified_base (476)..(476): N1-methylpseudouracil
  modified_base (484)..(484): N1-methylpseudouracil
  modified_base (488)..(488): N1-methylpseudouracil
  modified_base (490)..(490): N1-methylpseudouracil
  modified_base (497)..(497): N1-methylpseudouracil
  modified_base (513)..(514): N1-methylpseudouracil
  modified_base (517)..(518): N1-methylpseudouracil
  modified_base (520)..(520): N1-methylpseudouracil
  modified_base (528)..(528): N1-methylpseudouracil
  modified_base (532)..(533): N1-methylpseudouracil
  modified_base (536)..(536): N1-methylpseudouracil
  modified_base (538)..(539): N1-methylpseudouracil
  modified_base (541)..(542): N1-methylpseudouracil
  modified_base (553)..(553): N1-methylpseudouracil
  modified_base (565)..(566): N1-methylpseudouracil
  modified_base (574)..(574): N1-methylpseudouracil
  modified_base (577)..(577): N1-methylpseudouracil
  modified_base (591)..(591): N1-methylpseudouracil
  modified_base (601)..(601): N1-methylpseudouracil
  modified_base (605)..(605): N1-methylpseudouracil
  modified_base (608)..(608): N1-methylpseudouracil
  modified_base (612)..(612): N1-methylpseudouracil
  modified_base (617)..(617): N1-methylpseudouracil
  modified_base (626)..(626): N1-methylpseudouracil
  modified_base (646)..(646): N1-methylpseudouracil
  modified_base (654)..(654): N1-methylpseudouracil
  modified_base (665)..(665): N1-methylpseudouracil
  modified_base (670)..(670): N1-methylpseudouracil
  modified_base (680)..(680): N1-methylpseudouracil
  modified_base (688)..(689): N1-methylpseudouracil
  modified_base (702)..(704): N1-methylpseudouracil
  modified_base (712)..(712): N1-methylpseudouracil
  modified_base (719)..(719): N1-methylpseudouracil
  modified_base (723)..(723): N1-methylpseudouracil
  modified_base (727)..(728): N1-methylpseudouracil
  modified_base (739)..(740): N1-methylpseudouracil
  modified_base (744)..(744): N1-methylpseudouracil
  modified_base (754)..(755): N1-methylpseudouracil
  modified_base (759)..(759): N1-methylpseudouracil
  modified_base (764)..(764): N1-methylpseudouracil
  modified_base (773)..(773): N1-methylpseudouracil
  modified_base (776)..(776): N1-methylpseudouracil
  modified_base (780)..(780): N1-methylpseudouracil
  modified_base (785)..(785): N1-methylpseudouracil
  modified_base (794)..(794): N1-methylpseudouracil
  modified_base (798)..(798): N1-methylpseudouracil
  modified_base (805)..(805): N1-methylpseudouracil
  modified_base (808)..(808): N1-methylpseudouracil
  modified_base (810)..(810): N1-methylpseudouracil
  modified_base (821)..(821): N1-methylpseudouracil
  modified_base (824)..(824): N1-methylpseudouracil
  modified_base (829)..(829): N1-methylpseudouracil
  modified_base (851)..(851): N1-methylpseudouracil
  modified_base (859)..(859): N1-methylpseudouracil
  modified_base (863)..(863): N1-methylpseudouracil
  modified_base (865)..(865): N1-methylpseudouracil
  modified_base (884)..(884): N1-methylpseudouracil
  modified_base (896)..(896): N1-methylpseudouracil
  modified_base (904)..(904): N1-methylpseudouracil
  modified_base (906)..(907): N1-methylpseudouracil
  modified_base (909)..(909): N1-methylpseudouracil
  modified_base (918)..(918): N1-methylpseudouracil
  modified_base (920)..(920): N1-methylpseudouracil
  modified_base (922)..(923): N1-methylpseudouracil
  modified_base (933)..(933): N1-methylpseudouracil
  modified_base (937)..(937): N1-methylpseudouracil
  modified_base (941)..(942): N1-methylpseudouracil
  modified_base (950)..(950): N1-methylpseudouracil
  modified_base (965)..(965): N1-methylpseudouracil
  modified_base (969)..(969): N1-methylpseudouracil
  modified_base (975)..(975): N1-methylpseudouracil
  modified_base (977)..(977): N1-methylpseudouracil
  modified_base (985)..(986): N1-methylpseudouracil
  modified_base (997)..(998): N1-methylpseudouracil
  modified_base (1001)..(1001): N1-methylpseudouracil
  modified_base (1016)..(1016): N1-methylpseudouracil
  modified_base (1018)..(1018): N1-methylpseudouracil
  modified_base (1027)..(1027): N1-methylpseudouracil
  modified_base (1046)..(1049): N1-methylpseudouracil
  modified_base (1052)..(1052): N1-methylpseudouracil
  modified_base (1058)..(1058): N1-methylpseudouracil
  modified_base (1060)..(1061): N1-methylpseudouracil
  modified_base (1064)..(1064): N1-methylpseudouracil
  modified_base (1066)..(1066): N1-methylpseudouracil
  modified_base (1072)..(1072): N1-methylpseudouracil
  modified_base (1086)..(1086): N1-methylpseudouracil
  modified_base (1093)..(1093): N1-methylpseudouracil
  modified_base (1100)..(1100): N1-methylpseudouracil
  modified_base (1103)..(1103): N1-methylpseudouracil
  modified_base (1106)..(1106): N1-methylpseudouracil
  modified_base (1109)..(1109): N1-methylpseudouracil
  modified_base (1112)..(1112): N1-methylpseudouracil
  modified_base (1118)..(1118): N1-methylpseudouracil
  modified_base (1122)..(1122): N1-methylpseudouracil
  modified_base (1129)..(1129): N1-methylpseudouracil
  modified_base (1137)..(1137): N1-methylpseudouracil
  modified_base (1142)..(1142): N1-methylpseudouracil
  modified_base (1150)..(1150): N1-methylpseudouracil
  modified_base (1153)..(1153): N1-methylpseudouracil
  modified_base (1170)..(1170): N1-methylpseudouracil
  modified_base (1177)..(1177): N1-methylpseudouracil
  modified_base (1179)..(1180): N1-methylpseudouracil
  modified_base (1190)..(1190): N1-methylpseudouracil
  modified_base (1192)..(1193): N1-methylpseudouracil
  modified_base (1201)..(1202): N1-methylpseudouracil
  modified_base (1209)..(1209): N1-methylpseudouracil
  modified_base (1211)..(1211): N1-methylpseudouracil
  modified_base (1214)..(1214): N1-methylpseudouracil
  modified_base (1224)..(1224): N1-methylpseudouracil
  modified_base (1232)..(1232): N1-methylpseudouracil
  modified_base (1235)..(1235): N1-methylpseudouracil
  modified_base (1246)..(1246): N1-methylpseudouracil
  modified_base (1248)..(1248): N1-methylpseudouracil
  modified_base (1253)..(1253): N1-methylpseudouracil
  modified_base (1255)..(1256): N1-methylpseudouracil
  modified_base (1265)..(1265): N1-methylpseudouracil
  modified_base (1273)..(1273): N1-methylpseudouracil
  modified_base (1279)..(1280): N1-methylpseudouracil
  modified_base (1292)..(1292): N1-methylpseudouracil
  modified_base (1294)..(1295): N1-methylpseudouracil
  modified_base (1298)..(1298): N1-methylpseudouracil
  modified_base (1303)..(1303): N1-methylpseudouracil
  modified_base (1316)..(1316): N1-methylpseudouracil
  modified_base (1320)..(1320): N1-methylpseudouracil
  modified_base (1325)..(1325): N1-methylpseudouracil
  modified_base (1329)..(1329): N1-methylpseudouracil
  modified_base (1335)..(1335): N1-methylpseudouracil
  modified_base (1340)..(1340): N1-methylpseudouracil
  modified_base (1343)..(1343): N1-methylpseudouracil
  modified_base (1349)..(1350): N1-methylpseudouracil
  modified_base (1353)..(1353): N1-methylpseudouracil
  modified_base (1361)..(1361): N1-methylpseudouracil
  modified_base (1370)..(1370): N1-methylpseudouracil
  modified_base (1381)..(1381): N1-methylpseudouracil
  modified_base (1384)..(1384): N1-methylpseudouracil
  modified_base (1386)..(1386): N1-methylpseudouracil
  modified_base (1401)..(1401): N1-methylpseudouracil
  modified_base (1409)..(1409): N1-methylpseudouracil
  modified_base (1413)..(1414): N1-methylpseudouracil
  modified_base (1426)..(1426): N1-methylpseudouracil
  modified_base (1430)..(1430): N1-methylpseudouracil
  modified_base (1432)..(1432): N1-methylpseudouracil
  modified_base (1436)..(1437): N1-methylpseudouracil
  modified_base (1439)..(1439): N1-methylpseudouracil
  modified_base (1442)..(1442): N1-methylpseudouracil
  modified_base (1451)..(1451): N1-methylpseudouracil
  modified_base (1453)..(1453): N1-methylpseudouracil
  modified_base (1457)..(1457): N1-methylpseudouracil
  modified_base (1460)..(1460): N1-methylpseudouracil
  modified_base (1476)..(1476): N1-methylpseudouracil
  modified_base (1478)..(1478): N1-methylpseudouracil
  modified_base (1490)..(1490): N1-methylpseudouracil
  modified_base (1498)..(1498): N1-methylpseudouracil
  modified_base (1500)..(1501): N1-methylpseudouracil
  modified_base (1513)..(1514): N1-methylpseudouracil
  modified_base (1517)..(1517): N1-methylpseudouracil
  modified_base (1521)..(1521): N1-methylpseudouracil
  modified_base (1530)..(1531): N1-methylpseudouracil
  modified_base (1534)..(1535): N1-methylpseudouracil
  modified_base (1544)..(1544): N1-methylpseudouracil
  modified_base (1550)..(1551): N1-methylpseudouracil
  modified_base (1560)..(1561): N1-methylpseudouracil
  modified_base (1565)..(1565): N1-methylpseudouracil
  modified_base (1569)..(1569): N1-methylpseudouracil
  modified_base (1576)..(1577): N1-methylpseudouracil
  modified_base (1590)..(1592): N1-methylpseudouracil
  modified_base (1597)..(1598): N1-methylpseudouracil
  modified_base (1610)..(1612): N1-methylpseudouracil
  modified_base (1617)..(1617): N1-methylpseudouracil
  modified_base (1619)..(1619): N1-methylpseudouracil
  modified_base (1640)..(1640): N1-methylpseudouracil
  modified_base (1658)..(1658): N1-methylpseudouracil
  modified_base (1661)..(1661): N1-methylpseudouracil
  modified_base (1666)..(1667): N1-methylpseudouracil
  modified_base (1670)..(1670): N1-methylpseudouracil
  modified_base (1703)..(1704): N1-methylpseudouracil
  modified_base (1712)..(1712): N1-methylpseudouracil
  modified_base (1715)..(1715): N1-methylpseudouracil
  modified_base (1723)..(1724): N1-methylpseudouracil
  modified_base (1732)..(1733): N1-methylpseudouracil
  modified_base (1736)..(1736): N1-methylpseudouracil
  modified_base (1744)..(1744): N1-methylpseudouracil
  modified_base (1746)..(1747): N1-methylpseudouracil
  modified_base (1749)..(1749): N1-methylpseudouracil
  modified_base (1770)..(1771): N1-methylpseudouracil
  modified_base (1774)..(1775): N1-methylpseudouracil
  modified_base (1795)..(1796): N1-methylpseudouracil
  modified_base (1799)..(1799): N1-methylpseudouracil
  modified_base (1807)..(1807): N1-methylpseudouracil
  modified_base (1809)..(1809): N1-methylpseudouracil
  modified_base (1815)..(1815): N1-methylpseudouracil
  modified_base (1819)..(1820): N1-methylpseudouracil
  modified_base (1826)..(1826): N1-methylpseudouracil
  modified_base (1830)..(1830): N1-methylpseudouracil
  modified_base (1845)..(1845): N1-methylpseudouracil
  modified_base (1855)..(1855): N1-methylpseudouracil
  modified_base (1874)..(1874): N1-methylpseudouracil
  modified_base (1880)..(1880): N1-methylpseudouracil
  modified_base (1889)..(1889): N1-methylpseudouracil
  modified_base (1892)..(1892): N1-methylpseudouracil
  modified_base (1895)..(1895): N1-methylpseudouracil
  modified_base (1900)..(1901): N1-methylpseudouracil
  modified_base (1907)..(1907): N1-methylpseudouracil
  modified_base (1910)..(1910): N1-methylpseudouracil
  modified_base (1913)..(1913): N1-methylpseudouracil
  modified_base (1922)..(1922): N1-methylpseudouracil
  modified_base (1930)..(1930): N1-methylpseudouracil
  modified_base (1946)..(1946): N1-methylpseudouracil
  modified_base (1955)..(1955): N1-methylpseudouracil
  modified_base (1958)..(1958): N1-methylpseudouracil
  modified_base (1963)..(1964): N1-methylpseudouracil
  modified_base (1969)..(1970): N1-methylpseudouracil
  modified_base (1972)..(1972): N1-methylpseudouracil
  modified_base (1976)..(1976): N1-methylpseudouracil
  modified_base (1980)..(1980): N1-methylpseudouracil
  modified_base (1992)..(1992): N1-methylpseudouracil
  modified_base (1997)..(1997): N1-methylpseudouracil
  modified_base (2006)..(2007): N1-methylpseudouracil
  modified_base (2011)..(2011): N1-methylpseudouracil
  modified_base (2015)..(2015): N1-methylpseudouracil
  modified_base (2027)..(2028): N1-methylpseudouracil
  modified_base (2036)..(2036): N1-methylpseudouracil
  modified_base (2048)..(2048): N1-methylpseudouracil
  modified_base (2054)..(2054): N1-methylpseudouracil
  modified_base (2061)..(2061): N1-methylpseudouracil
  modified_base (2064)..(2064): N1-methylpseudouracil
  modified_base (2070)..(2072): N1-methylpseudouracil
  modified_base (2078)..(2078): N1-methylpseudouracil
  modified_base (2085)..(2085): N1-methylpseudouracil
  modified_base (2093)..(2094): N1-methylpseudouracil
  modified_base (2096)..(2097): N1-methylpseudouracil
  modified_base (2105)..(2105): N1-methylpseudouracil
  modified_base (2111)..(2111): N1-methylpseudouracil
  modified_base (2114)..(2114): N1-methylpseudouracil
  modified_base (2117)..(2117): N1-methylpseudouracil
  modified_base (2120)..(2120): N1-methylpseudouracil
  modified_base (2125)..(2126): N1-methylpseudouracil
  modified_base (2134)..(2134): N1-methylpseudouracil
  modified_base (2146)..(2147): N1-methylpseudouracil
  modified_base (2150)..(2150): N1-methylpseudouracil
  modified_base (2152)..(2152): N1-methylpseudouracil
  modified_base (2158)..(2158): N1-methylpseudouracil
  modified_base (2180)..(2180): N1-methylpseudouracil
  modified_base (2183)..(2183): N1-methylpseudouracil
  modified_base (2188)..(2189): N1-methylpseudouracil
  modified_base (2192)..(2192): N1-methylpseudouracil
  modified_base (2200)..(2200): N1-methylpseudouracil
  modified_base (2204)..(2204): N1-methylpseudouracil
  modified_base (2209)..(2210): N1-methylpseudouracil
  modified_base (2212)..(2212): N1-methylpseudouracil
  modified_base (2222)..(2222): N1-methylpseudouracil
  modified_base (2225)..(2225): N1-methylpseudouracil
  modified_base (2227)..(2227): N1-methylpseudouracil
  modified_base (2229)..(2229): N1-methylpseudouracil
  modified_base (2232)..(2232): N1-methylpseudouracil
  modified_base (2234)..(2234): N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 37 - RNA, 2514 nt, Artificial Sequence
  source: /note=“Description of Artificial Sequence: Synthetic polynucleotide”
  modified_base (7)..(7): N1-methylpseudouracil
  modified_base (25)..(25): N1-methylpseudouracil
  modified_base (35)..(35): N1-methylpseudouracil
  modified_base (37)..(37): N1-methylpseudouracil
  modified_base (59)..(59): N1-methylpseudouracil
  modified_base (64)..(64): N1-methylpseudouracil
  modified_base (68)..(68): N1-methylpseudouracil
  modified_base (73)..(74): N1-methylpseudouracil
  modified_base (77)..(77): N1-methylpseudouracil
  modified_base (83)..(83): N1-methylpseudouracil
  modified_base (86)..(86): N1-methylpseudouracil
  modified_base (88)..(89): N1-methylpseudouracil
  modified_base (92)..(92): N1-methylpseudouracil
  modified_base (94)..(95): N1-methylpseudouracil
  modified_base (106)..(106): N1-methylpseudouracil
  modified_base (119)..(119): N1-methylpseudouracil
  modified_base (121)..(122): N1-methylpseudouracil
  modified_base (149)..(149): N1-methylpseudouracil
  modified_base (160)..(161): N1-methylpseudouracil
  modified_base (170)..(170): N1-methylpseudouracil
  modified_base (184)..(185): N1-methylpseudouracil
  modified_base (194)..(194): N1-methylpseudouracil
  modified_base (197)..(197): N1-methylpseudouracil
  modified_base (200)..(200): N1-methylpseudouracil
  modified_base (203)..(203): N1-methylpseudouracil
  modified_base (208)..(209): N1-methylpseudouracil
  modified_base (217)..(217): N1-methylpseudouracil
  modified_base (221)..(221): N1-methylpseudouracil
  modified_base (229)..(229): N1-methylpseudouracil
  modified_base (234)..(236): N1-methylpseudouracil
  modified_base (248)..(248): N1-methylpseudouracil
  modified_base (254)..(254): N1-methylpseudouracil
  modified_base (257)..(257): N1-methylpseudouracil
  modified_base (266)..(266): N1-methylpseudouracil
  modified_base (274)..(275): N1-methylpseudouracil
  modified_base (286)..(286): N1-methylpseudouracil
  modified_base (290)..(290): N1-methylpseudouracil
  modified_base (313)..(313): N1-methylpseudouracil
  modified_base (326)..(326): N1-methylpseudouracil
  modified_base (335)..(335): N1-methylpseudouracil
  modified_base (337)..(338): N1-methylpseudouracil
  modified_base (350)..(350): N1-methylpseudouracil
  modified_base (352)..(352): N1-methylpseudouracil
  modified_base (359)..(359): N1-methylpseudouracil
  modified_base (368)..(368): N1-methylpseudouracil
  modified_base (378)..(379): N1-methylpseudouracil
  modified_base (389)..(389): N1-methylpseudouracil
  modified_base (397)..(397): N1-methylpseudouracil
  modified_base (400)..(400): N1-methylpseudouracil
  modified_base (430)..(430): N1-methylpseudouracil
  modified_base (433)..(434): N1-methylpseudouracil
  modified_base (437)..(437): N1-methylpseudouracil
  modified_base (459)..(459): N1-methylpseudouracil
  modified_base (464)..(464): N1-methylpseudouracil
  modified_base (468)..(468): N1-methylpseudouracil
  modified_base (473)..(473): N1-methylpseudouracil
  modified_base (476)..(476): N1-methylpseudouracil
  modified_base (483)..(483): N1-methylpseudouracil
  modified_base (488)..(488): N1-methylpseudouracil
  modified_base (494)..(494): N1-methylpseudouracil
  modified_base (497)..(497): N1-methylpseudouracil
  modified_base (499)..(499): N1-methylpseudouracil
  modified_base (501)..(501): N1-methylpseudouracil
  modified_base (508)..(509): N1-methylpseudouracil
  modified_base (529)..(530): N1-methylpseudouracil
  modified_base (533)..(533): N1-methylpseudouracil
  modified_base (541)..(541): N1-methylpseudouracil
  modified_base (545)..(545): N1-methylpseudouracil
  modified_base (547)..(547): N1-methylpseudouracil
  modified_base (554)..(554): N1-methylpseudouracil
  modified_base (570)..(571): N1-methylpseudouracil
  modified_base (574)..(575): N1-methylpseudouracil
  modified_base (577)..(577): N1-methylpseudouracil
  modified_base (585)..(585): N1-methylpseudouracil
  modified_base (589)..(590): N1-methylpseudouracil
  modified_base (593)..(593): N1-methylpseudouracil
  modified_base (595)..(596): N1-methylpseudouracil
  modified_base (598)..(599): N1-methylpseudouracil
  modified_base (610)..(610): N1-methylpseudouracil
  modified_base (622)..(623): N1-methylpseudouracil
  modified_base (631)..(631): N1-methylpseudouracil
  modified_base (634)..(634): N1-methylpseudouracil
  modified_base (648)..(648): N1-methylpseudouracil
  modified_base (658)..(658): N1-methylpseudouracil
  modified_base (662)..(662): N1-methylpseudouracil
  modified_base (665)..(665): N1-methylpseudouracil
  modified_base (669)..(669): N1-methylpseudouracil
  modified_base (674)..(674): N1-methylpseudouracil
  modified_base (683)..(683): N1-methylpseudouracil
  modified_base (703)..(703): N1-methylpseudouracil
  modified_base (711)..(711): N1-methylpseudouracil
  modified_base (722)..(722): N1-methylpseudouracil
  modified_base (727)..(727): N1-methylpseudouracil
  modified_base (737)..(737): N1-methylpseudouracil
  modified_base (745)..(746): N1-methylpseudouracil
  modified_base (759)..(761): N1-methylpseudouracil
  modified_base (769)..(769): N1-methylpseudouracil
  modified_base (776)..(776): N1-methylpseudouracil
  modified_base (780)..(780): N1-methylpseudouracil
  modified_base (784)..(785): N1-methylpseudouracil
  modified_base (796)..(797): N1-methylpseudouracil
  modified_base (801)..(801): N1-methylpseudouracil
  modified_base (811)..(812): N1-methylpseudouracil
  modified_base (816)..(816): N1-methylpseudouracil
  modified_base (821)..(821): N1-methylpseudouracil
  modified_base (830)..(830): N1-methylpseudouracil
  modified_base (833)..(833): N1-methylpseudouracil
  modified_base (837)..(837): N1-methylpseudouracil
  modified_base (842)..(842): N1-methylpseudouracil
  modified_base (851)..(851): N1-methylpseudouracil
  modified_base (855)..(855): N1-methylpseudouracil
  modified_base (862)..(862): N1-methylpseudouracil
  modified_base (865)..(865): N1-methylpseudouracil
  modified_base (867)..(867): N1-methylpseudouracil
  modified_base (878)..(878): N1-methylpseudouracil
  modified_base (881)..(881): N1-methylpseudouracil
  modified_base (886)..(886): N1-methylpseudouracil
  modified_base (908)..(908): N1-methylpseudouracil
  modified_base (916)..(916): N1-methylpseudouracil
  modified_base (920)..(920): N1-methylpseudouracil
  modified_base (922)..(922): N1-methylpseudouracil
  modified_base (941)..(941): N1-methylpseudouracil
  modified_base (953)..(953): N1-methylpseudouracil
  modified_base (961)..(961): N1-methylpseudouracil
  modified_base (963)..(964): N1-methylpseudouracil
  modified_base (966)..(966): N1-methylpseudouracil
  modified_base (975)..(975): N1-methylpseudouracil
  modified_base (977)..(977): N1-methylpseudouracil
  modified_base (979)..(980): N1-methylpseudouracil
  modified_base (990)..(990): N1-methylpseudouracil
  modified_base (994)..(994): N1-methylpseudouracil
  modified_base (998)..(999): N1-methylpseudouracil
  modified_base (1007)..(1007): N1-methylpseudouracil
  modified_base (1022)..(1022): N1-methylpseudouracil
  modified_base (1026)..(1026): N1-methylpseudouracil
  modified_base (1032)..(1032): N1-methylpseudouracil
  modified_base (1034)..(1034): N1-methylpseudouracil
  modified_base (1042)..(1043): N1-methylpseudouracil
  modified_base (1054)..(1055): N1-methylpseudouracil
  modified_base (1058)..(1058): N1-methylpseudouracil
  modified_base (1073)..(1073): N1-methylpseudouracil
  modified_base (1075)..(1075): N1-methylpseudouracil
  modified_base (1084)..(1084): N1-methylpseudouracil
  modified_base (1103)..(1106): N1-methylpseudouracil
  modified_base (1109)..(1109): N1-methylpseudouracil
  modified_base (1115)..(1115): N1-methylpseudouracil
  modified_base (1117)..(1118): N1-methylpseudouracil
  modified_base (1121)..(1121): N1-methylpseudouracil
  modified_base (1123)..(1123): N1-methylpseudouracil
  modified_base (1129)..(1129): N1-methylpseudouracil
  modified_base (1143)..(1143): N1-methylpseudouracil
  modified_base (1150)..(1150): N1-methylpseudouracil
  modified_base (1157)..(1157): N1-methylpseudouracil
  modified_base (1160)..(1160): N1-methylpseudouracil
  modified_base (1163)..(1163): N1-methylpseudouracil
  modified_base (1166)..(1166): N1-methylpseudouracil
  modified_base (1169)..(1169): N1-methylpseudouracil
  modified_base (1175)..(1175): N1-methylpseudouracil
  modified_base (1179)..(1179): N1-methylpseudouracil
  modified_base (1186)..(1186): N1-methylpseudouracil
  modified_base (1194)..(1194): N1-methylpseudouracil
  modified_base (1199)..(1199): N1-methylpseudouracil
  modified_base (1207)..(1207): N1-methylpseudouracil
  modified_base (1210)..(1210): N1-methylpseudouracil
  modified_base (1227)..(1227): N1-methylpseudouracil
  modified_base (1234)..(1234): N1-methylpseudouracil
  modified_base (1236)..(1237): N1-methylpseudouracil
  modified_base (1247)..(1247): N1-methylpseudouracil
  modified_base (1249)..(1250): N1-methylpseudouracil
  modified_base (1258)..(1259): N1-methylpseudouracil
  modified_base (1266)..(1266): N1-methylpseudouracil
  modified_base (1268)..(1268): N1-methylpseudouracil
  modified_base (1271)..(1271): N1-methylpseudouracil
  modified_base (1281)..(1281): N1-methylpseudouracil
  modified_base (1289)..(1289): N1-methylpseudouracil
  modified_base (1292)..(1292): N1-methylpseudouracil
  modified_base (1303)..(1303): N1-methylpseudouracil
  modified_base (1305)..(1305): N1-methylpseudouracil
  modified_base (1310)..(1310): N1-methylpseudouracil
  modified_base (1312)..(1313): N1-methylpseudouracil
  modified_base (1322)..(1322): N1-methylpseudouracil
  modified_base (1330)..(1330): N1-methylpseudouracil
  modified_base (1336)..(1337): N1-methylpseudouracil
  modified_base (1349)..(1349): N1-methylpseudouracil
  modified_base (1351)..(1352): N1-methylpseudouracil
  modified_base (1355)..(1355): N1-methylpseudouracil
  modified_base (1360)..(1360): N1-methylpseudouracil
  modified_base (1373)..(1373): N1-methylpseudouracil
  modified_base (1377)..(1377): N1-methylpseudouracil
  modified_base (1382)..(1382): N1-methylpseudouracil
  modified_base (1386)..(1386): N1-methylpseudouracil
  modified_base (1392)..(1392): N1-methylpseudouracil
  modified_base (1397)..(1397): N1-methylpseudouracil
  modified_base (1400)..(1400): N1-methylpseudouracil
  modified_base (1406)..(1407): N1-methylpseudouracil
  modified_base (1410)..(1410): N1-methylpseudouracil
  modified_base (1418)..(1418): N1-methylpseudouracil
  modified_base (1427)..(1427): N1-methylpseudouracil
  modified_base (1438)..(1438): N1-methylpseudouracil
  modified_base (1441)..(1441): N1-methylpseudouracil
  modified_base (1443)..(1443): N1-methylpseudouracil
  modified_base (1458)..(1458): N1-methylpseudouracil
  modified_base (1466)..(1466): N1-methylpseudouracil
  modified_base (1470)..(1471): N1-methylpseudouracil
  modified_base (1483)..(1483): N1-methylpseudouracil
  modified_base (1487)..(1487): N1-methylpseudouracil
  modified_base (1489)..(1489): N1-methylpseudouracil
  modified_base (1493)..(1494): N1-methylpseudouracil
  modified_base (1496)..(1496): N1-methylpseudouracil
  modified_base (1499)..(1499): N1-methylpseudouracil
  modified_base (1508)..(1508): N1-methylpseudouracil
  modified_base (1510)..(1510): N1-methylpseudouracil
  modified_base (1514)..(1514): N1-methylpseudouracil
  modified_base (1517)..(1517): N1-methylpseudouracil
  modified_base (1533)..(1533): N1-methylpseudouracil
  modified_base (1535)..(1535): N1-methylpseudouracil
  modified_base (1547)..(1547): N1-methylpseudouracil
  modified_base (1555)..(1555): N1-methylpseudouracil
  modified_base (1557)..(1558): N1-methylpseudouracil
  modified_base (1570)..(1571): N1-methylpseudouracil
  modified_base (1574)..(1574): N1-methylpseudouracil
  modified_base (1578)..(1578): N1-methylpseudouracil
  modified_base (1587)..(1588): N1-methylpseudouracil
  modified_base (1591)..(1592): N1-methylpseudouracil
  modified_base (1601)..(1601): N1-methylpseudouracil
  modified_base (1607)..(1608): N1-methylpseudouracil
  modified_base (1617)..(1618): N1-methylpseudouracil
  modified_base (1622)..(1622): N1-methylpseudouracil
  modified_base (1626)..(1626): N1-methylpseudouracil
  modified_base (1633)..(1634): N1-methylpseudouracil
  modified_base (1647)..(1649): N1-methylpseudouracil
  modified_base (1654)..(1655): N1-methylpseudouracil
  modified_base (1667)..(1669): N1-methylpseudouracil
  modified_base (1674)..(1674): N1-methylpseudouracil
  modified_base (1676)..(1676): N1-methylpseudouracil
  modified_base (1697)..(1697): N1-methylpseudouracil
  modified_base (1715)..(1715): N1-methylpseudouracil
  modified_base (1718)..(1718): N1-methylpseudouracil
  modified_base (1723)..(1724): N1-methylpseudouracil
  modified_base (1727)..(1727): N1-methylpseudouracil
  modified_base (1760)..(1761): N1-methylpseudouracil
  modified_base (1769)..(1769): N1-methylpseudouracil
  modified_base (1772)..(1772): N1-methylpseudouracil
  modified_base (1780)..(1781): N1-methylpseudouracil
  modified_base (1789)..(1790): N1-methylpseudouracil
  modified_base (1793)..(1793): N1-methylpseudouracil
  modified_base (1801)..(1801): N1-methylpseudouracil
  modified_base (1803)..(1804): N1-methylpseudouracil
  modified_base (1806)..(1806): N1-methylpseudouracil
  modified_base (1827)..(1828): N1-methylpseudouracil
  modified_base (1831)..(1832): N1-methylpseudouracil
  modified_base (1852)..(1853): N1-methylpseudouracil
  modified_base (1856)..(1856): N1-methylpseudouracil
  modified_base (1864)..(1864): N1-methylpseudouracil
  modified_base (1866)..(1866): N1-methylpseudouracil
  modified_base (1872)..(1872): N1-methylpseudouracil
  modified_base (1876)..(1877): N1-methylpseudouracil
  modified_base (1883)..(1883): N1-methylpseudouracil
  modified_base (1887)..(1887): N1-methylpseudouracil
  modified_base (1902)..(1902): N1-methylpseudouracil
  modified_base (1912)..(1912): N1-methylpseudouracil
  modified_base (1931)..(1931): N1-methylpseudouracil
  modified_base (1937)..(1937): N1-methylpseudouracil
  modified_base (1946)..(1946): N1-methylpseudouracil
  modified_base (1949)..(1949): N1-methylpseudouracil
  modified_base (1952)..(1952): N1-methylpseudouracil
  modified_base (1957)..(1958): N1-methylpseudouracil
  modified_base (1964)..(1964): N1-methylpseudouracil
  modified_base (1967)..(1967): N1-methylpseudouracil
  modified_base (1970)..(1970): N1-methylpseudouracil
  modified_base (1979)..(1979): N1-methylpseudouracil
  modified_base (1987)..(1987): N1-methylpseudouracil
  modified_base (2003)..(2003): N1-methylpseudouracil
  modified_base (2012)..(2012): N1-methylpseudouracil
  modified_base (2015)..(2015): N1-methylpseudouracil
  modified_base (2020)..(2021): N1-methylpseudouracil
  modified_base (2026)..(2027): N1-methylpseudouracil
  modified_base (2029)..(2029): N1-methylpseudouracil
  modified_base (2033)..(2033): N1-methylpseudouracil
  modified_base (2037)..(2037): N1-methylpseudouracil
  modified_base (2049)..(2049): N1-methylpseudouracil
  modified_base (2054)..(2054): N1-methylpseudouracil
  modified_base (2063)..(2064): N1-methylpseudouracil
  modified_base (2068)..(2068): N1-methylpseudouracil
  modified_base (2072)..(2072): N1-methylpseudouracil
  modified_base (2084)..(2085): N1-methylpseudouracil
  modified_base (2093)..(2093): N1-methylpseudouracil
  modified_base (2105)..(2105): N1-methylpseudouracil
  modified_base (2111)..(2111): N1-methylpseudouracil
  modified_base (2118)..(2118): N1-methylpseudouracil
  modified_base (2121)..(2121): N1-methylpseudouracil
  modified_base (2127)..(2129): N1-methylpseudouracil
  modified_base (2135)..(2135): N1-methylpseudouracil
  modified_base (2142)..(2142): N1-methylpseudouracil
  modified_base (2150)..(2151): N1-methylpseudouracil
  modified_base (2153)..(2154): N1-methylpseudouracil
  modified_base (2162)..(2162): N1-methylpseudouracil
  modified_base (2168)..(2168): N1-methylpseudouracil
  modified_base (2171)..(2171): N1-methylpseudouracil
  modified_base (2174)..(2174): N1-methylpseudouracil
  modified_base (2177)..(2177): N1-methylpseudouracil
  modified_base (2182)..(2183): N1-methylpseudouracil
  modified_base (2191)..(2191): N1-methylpseudouracil
  modified_base (2203)..(2204): N1-methylpseudouracil
  modified_base (2207)..(2207): N1-methylpseudouracil
  modified_base (2209)..(2209): N1-methylpseudouracil
  modified_base (2215)..(2215): N1-methylpseudouracil
  modified_base (2237)..(2237): N1-methylpseudouracil
  modified_base (2240)..(2240): N1-methylpseudouracil
  modified_base (2245)..(2246): N1-methylpseudouracil
  modified_base (2249)..(2249): N1-methylpseudouracil
  modified_base (2257)..(2257): N1-methylpseudouracil
  modified_base (2261)..(2261): N1-methylpseudouracil
  modified_base (2266)..(2267): N1-methylpseudouracil
  modified_base (2269)..(2269): N1-methylpseudouracil
  modified_base (2279)..(2279): N1-methylpseudouracil
  modified_base (2282)..(2282): N1-methylpseudouracil
  modified_base (2284)..(2284): N1-methylpseudouracil
  modified_base (2286)..(2286): N1-methylpseudouracil
  modified_base (2289)..(2289): N1-methylpseudouracil
  modified_base (2291)..(2291): N1-methylpseudouracil
  modified_base (2296)..(2296): N1-methylpseudouracil
  modified_base (2299)..(2299): N1-methylpseudouracil
  modified_base (2302)..(2302): N1-methylpseudouracil
  modified_base (2307)..(2307): N1-methylpseudouracil
  modified_base (2314)..(2314): N1-methylpseudouracil
  modified_base (2318)..(2318): N1-methylpseudouracil
  modified_base (2323)..(2323): N1-methylpseudouracil
  modified_base (2327)..(2328): N1-methylpseudouracil
  modified_base (2330)..(2331): N1-methylpseudouracil
  modified_base (2337)..(2338): N1-methylpseudouracil
  modified_base (2344)..(2344): N1-methylpseudouracil
  modified_base (2357)..(2357): N1-methylpseudouracil
  modified_base (2360)..(2360): N1-methylpseudouracil
  modified_base (2365)..(2366): N1-methylpseudouracil
  modified_base (2369)..(2369): N1-methylpseudouracil
  modified_base (2377)..(2377): N1-methylpseudouracil
  modified_base (2385)..(2385): N1-methylpseudouracil
  modified_base (2388)..(2388): N1-methylpseudouracil
  modified_base (2390)..(2392): N1-methylpseudouracil
  modified_base (2396)..(2396): N1-methylpseudouracil
  modified_base (2401)..(2401): N1-methylpseudouracil
  modified_base (2403)..(2403): N1-methylpseudouracil
  modified_base (2407)..(2407): N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - RNA, 432 nt, Homo sapiens
auguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggag

SEQ ID NO: 39 - protein, 141 aa, Mus sp.
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 40 - RNA, 423 nt, Mus sp.
auguggcuucagaaucucuuguuucuuggaaucgucguguacagccugucagccccaacuagaucgccuaucacugugacgcgcccguggaagcacguggaagccaucaaggaggcucugaaucugcucgacgauaugccagugacccugaacgaggaagucgaagugguguccaacgaauuuuccuucaagaaguugaccuguguucagacccggcugaagauuuucgagcagggccucaggggaaacuucaccaaacugaagggugcacugaacaugaccgccagcuacuaccagaccuauugcccuccgacuccggaaacugauugcgagacucaagucaccaccuacgcggacuucaucgacucgcucaagacguuccugacugacauccccuucgagugcaagaagccggggcagaaa

SEQ ID NO: 41 - protein, 144 aa, Rattus sp.
MWLQNLLFLGIVVYSFSAPTRSPNPVTRPWKHVDAIKEALSLLNDMRALENEKNEDVDIISNEFSIQRPTCVQTRLKLYKQGLRGNLTKLNGALTMIASHYQTNCPPTPETDCEIEVTTFEDFIKNLKGFLFDIPFDCWKPVQK

SEQ ID NO: 42 - RNA, 432 nt, Rattus sp.
auguggcugcagaaccugcuguuccugggcaucgugguguacagcuucagcgccccuaccagaagcccuaacccugugaccagaccuuggaagcacguggacgccaucaaggaggcccugagccugcugaacgacaugagagcccuggagaacgagaagaacgaggacguggacaucaucagcaacgaguucagcauccagagaccuaccugcgugcagaccagacugaagcuguacaagcagggccugagaggcaaccugaccaagcugaacggcgcccugaccaugaucgccagccacuaccagaccaacugcccuccuaccccugagaccgacugcgagaucgaggugaccaccuucgaggacuucaucaagaaccugaagggcuuccuguucgacaucccuuucgacugcuggaagccugugcagaag

SEQ ID NO: 43 - protein, 144 aa, Macaca fascicularis
MWLQGLLLLGTVACSISAPARSPSPGTQPWEHVNAIQEARRLLNLSRDTAAEMNKTVEVVSEMFDLQEPSCLQTRLELYKQGLQGSLTKLKGPLTMMASHYKQHCPPTPETSCATQIITFQSFKENLKDFLLVIPFDCWEPVQE

SEQ ID NO: 44 - RNA, 432 nt, Macaca fascicularis
auguggcugcagggccugcugcugcugggcaccguggccugcagcaucagcgccccugccagaagcccuagcccuggcacccagccuugggagcacgugaacgccauccaggaggccagaagacugcugaaccugagcagagacaccgccgccgagaugaacaagaccguggagguggugagcgagauguucgaccugcaggagccuagcugccugcagaccagacuggagcuguacaagcagggccugcagggcagccugaccaagcugaagggcccucugaccaugauggccagccacuacaagcagcacugcccuccuaccccugagaccagcugcgccacccagaucaucaccuuccagagcuucaaggagaaccugaaggacuuccugcuggugaucccuuucgacugcugggagccugugcaggag

SEQ ID NO: 45-86 - skipped (42 entries)

SEQ ID NO: 87 - RNA, 9 nt, Unknown
  source: /note=“Description of Unknown: Kozak sequence”
ccrccaugg

SEQ ID NO: 88 - RNA, 92 nt, Unknown
  source: /note=“Description of Unknown: 5′ UTR-018 (Upstream UTR) 5′ UTR sequence”
ucaagcuuuuggacccucguacagaagcuaauacgacucacuauagggaaauaagagagaaaagaagaguaagaagaaauauaagagccacc

SEQ ID NO: 89 - RNA, 47 nt, Unknown
  source: /note=“Description of Unknown: 5′ UTR-002 (Upstream UTR) sequence”
gggagaucagagagaaaagaagaguaagaagaaauauaagagccacc

SEQ ID NO: 90 - RNA, 42 nt, Unknown
  source: /note=“Description of Unknown: 5′ UTR-004 (Upstream UTR) sequence”
gggagacaagcuuggcauuccgguacuguugguaaagccacc

SEQ ID NO: 91 - RNA, 47 nt, Unknown; identical to SEQ ID NO: 89
  source: /note=“Description of Unknown: 5′ UTR-005 (Upstream UTR) sequence”

SEQ ID NO: 92 - RNA, 42 nt, Unknown; identical to SEQ ID NO: 90
  source: /note=“Description of Unknown: 5′ UTR-007 (Upstream UTR) sequence”

SEQ ID NO: 93 - RNA, 47 nt, Unknown
  source: /note=“Description of Unknown: 5′ UTR-008 (Upstream UTR) sequence”
gggaauuaacagagaaaagaagaguaagaagaaauauaagagccacc

SEQ ID NO: 94 - RNA, 47 nt, Unknown
  source: /note=“Description of Unknown: 5′ UTR-009 (Upstream UTR) sequence”
gggaaauuagacagaaaagaagaguaagaagaaauauaagagccacc

SEQ ID NO: 95 - RNA, 47 nt, Unknown
  source: /note=“Description of Unknown: 5′ UTR-010, Upstream sequence”
gggaaauaagagaguaaagaacaguaagaagaaauauaagagccacc

SEQ ID NO: 96 - RNA, 47 nt, Unknown
  source: /note=“Description of Unknown: 5′ UTR-011 (Upstream UTR) sequence”
gggaaaaaagagagaaaagaagacuaagaagaaauauaagagccacc

SEQ ID NO: 97 - RNA, 47 nt, Unknown
  source: /note=“Description of Unknown: 5′ UTR-012 (Upstream UTR) sequence”
gggaaauaagagagaaaagaagaguaagaagauauauaagagccacc

SEQ ID NO: 98 - RNA, 47 nt, Unknown
  source: /note=“Description of Unknown: 5′ UTR-013 (Upstream UTR) sequence”
gggaaauaagagacaaaacaagaguaagaagaaauauaagagccacc

SEQ ID NO: 99 - RNA, 47 nt, Unknown
  source: /note=“Description of Unknown: 5′ UTR-014 (Upstream UTR) sequence”
gggaaauuagagaguaaagaacaguaaguagaauuaaaagagccacc

SEQ ID NO: 100 - RNA, 47 nt, Unknown
  source: /note=“Description of Unknown: 5′ UTR-015 (Upstream UTR) sequence”
gggaaauaagagagaauagaagaguaagaagaaauauaagagccacc

SEQ ID NO: 101 - RNA, 47 nt, Unknown
  source: /note=“Description of Unknown: 5′ UTR-016 (Upstream UTR) sequence”
gggaaauaagagagaaaagaagaguaagaagaaaauuaagagccacc

SEQ ID NO: 102 - RNA, 47 nt, Unknown
  source: /note=“Description of Unknown: 5′ UTR-017 (Upstream UTR) sequence”
gggaaauaagagagaaaagaagaguaagaagaaauuuaagagccacc

SEQ ID NO: 103 - skipped

SEQ ID NO: 104 - RNA, 142 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 105 - RNA, 142 nt, Artificial Sequence
  source: /note=“Description of Artificial Sequence: Synthetic polynucleotide”
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 106 - RNA, 142 nt, Artificial Sequence
  source: /note=“Description of Artificial Sequence: Synthetic polynucleotide”
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 107 - RNA, 142 nt, Artificial Sequence
  source: /note=“Description of Artificial Sequence: Synthetic polynucleotide”
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 108 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 109 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 110 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 111 - RNA, 164 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 112 - RNA, 164 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 113 - skipped

SEQ ID NO: 114 - RNA, 87 nt, Unknown
  source: /note=“Description of Unknown: miR-142 sequence”
gacagugcagucacccauaaaguagaaagcacuacuaacagcacuggaggguguaguguuuccuacuuuauggaugaguguacugug

SEQ ID NO: 115 - RNA, 23 nt, Unknown
  source: /note=“Description of Unknown: miR 142-3p sequence”
uguaguguuuccuacuuuaugga

SEQ ID NO: 116 - RNA, 23 nt, Unknown
  source: /note=“Description of Unknown: miR-142-3p binding site sequence”
uccauaaaguaggaaacacuaca

SEQ ID NO: 117 - RNA, 21 nt, Unknown
  source: /note=“Description of Unknown: miR-142-5p sequence”
cauaaaguagaaagcacuacu

SEQ ID NO: 118 - RNA, 21 nt, Unknown
  source: /note=“Description of Unknown: miR-142-5p binding site sequence”
aguagugcuuucuacuuuaug

SEQ ID NO: 119 - RNA, 85 nt, Unknown
  source: /note=“Description of Unknown: miR-126 sequence”
cgcuggcgacgggacauuauuacuuuugguacgcgcugugacacuucaaacucguaccgugaguaauaaugcgccguccacggca

SEQ ID NO: 120 - RNA, 22 nt, Unknown
  source: /note=“Description of Unknown: miR 126-3p sequence”
ucguaccgugaguaauaaugcg

SEQ ID NO: 121 - RNA, 22 nt, Unknown
  source: /note=“Description of Unknown: miR 126-3p binding site sequence”
cgcauuauuacucacgguacga

SEQ ID NO: 122 - RNA, 21 nt, Unknown
  source: /note=“Description of Unknown: miR 126-5p sequence”
cauuauuacuuuugguacgcg

SEQ ID NO: 123 - RNA, 21 nt, Unknown
  source: /note=“Description of Unknown: miR-126-5p binding site sequence”
cgcguaccaaaaguaauaaug

SEQ ID NO: 124 - RNA, 9 nt, Artificial Sequence
  source: /note=“Description of Artificial Sequence: Synthetic oligonucleotide”
ugauaauag

SEQ ID NO: 125 - RNA, 9 nt, Artificial Sequence
  source: /note=“Description of Artificial Sequence: Synthetic oligonucleotide”
ugauaguaa

SEQ ID NO: 126 - RNA, 9 nt, Artificial Sequence
  source: /note=“Description of Artificial Sequence: Synthetic oligonucleotide”
uaaugauag

SEQ ID NO: 127 - RNA, 9 nt, Artificial Sequence
  source: /note=“Description of Artificial Sequence: Synthetic oligonucleotide”
ugauaauaa

SEQ ID NO: 128 - RNA, 9 nt, Artificial Sequence
  source: /note=“Description of Artificial Sequence: Synthetic oligonucleotide”
ugauaguag

SEQ ID NO: 129 - RNA, 9 nt, Artificial Sequence
  source: /note=“Description of Artificial Sequence: Synthetic oligonucleotide”
uaaugauga

SEQ ID NO: 130 - RNA, 9 nt, Artificial Sequence
  source: /note=“Description of Artificial Sequence: Synthetic oligonucleotide”
uaauaguag

SEQ ID NO: 131 - RNA, 9 nt, Artificial Sequence
  source: /note=“Description of Artificial Sequence: Synthetic oligonucleotide”
ugaugauga

SEQ ID NO: 132 - RNA, 9 nt, Artificial Sequence
  source: /note=“Description of Artificial Sequence: Synthetic oligonucleotide”
uaauaauaa

SEQ ID NO: 133 - RNA, 9 nt, Artificial Sequence
  source: /note=“Description of Artificial Sequence: Synthetic oligonucleotide”
uaguaguag

SEQ ID NO: 134 - RNA, 133 nt, Artificial Sequence
  source: /note=“Description of Artificial Sequence: Synthetic polynucleotide”
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 135 - RNA, 22 nt, Unknown
  source: /note=“Description of Unknown: miR 146-3p sequence”
ccucugaaauucaguucuucag

SEQ ID NO: 136 - RNA, 22 nt, Unknown
  source: /note=“Description of Unknown: miR 146-5p sequence”
ugagaacugaauuccauggguu

SEQ ID NO: 137 - RNA, 22 nt, Unknown
  source: /note=“Description of Unknown: miR 155-3p sequence”
cuccuacauauuagcauuaaca

SEQ ID NO: 138 - RNA, 23 nt, Unknown
  source: /note=“Description of Unknown: miR 155-5p sequence”
uuaaugcuaaucgugauaggggu

SEQ ID NO: 139 - RNA, 22 nt, Unknown
  source: /note=“Description of Unknown: miR 16-3p sequence”
ccaguauuaacugugcugcuga

SEQ ID NO: 140 - RNA, 22 nt, Unknown
  source: /note=“Description of Unknown: miR 16-5p sequence”
uagcagcacguaaauauuggcg

SEQ ID NO: 141 - RNA, 21 nt, Unknown
  source: /note=“Description of Unknown: miR 21-3p sequence”
caacaccagucgaugggcugu

SEQ ID NO: 142 - RNA, 22 nt, Unknown
  source: /note=“Description of Unknown: miR 21-5p sequence”
uagcuuaucagacugauguuga

SEQ ID NO: 143 - RNA, 22 nt, Unknown
  source: /note=“Description of Unknown: miR 223-3p sequence”
ugucaguuugucaaauacccca

SEQ ID NO: 144 - RNA, 22 nt, Unknown
  source: /note=“Description of Unknown: miR 223-5p sequence”
cguguauuugacaagcugaguu

SEQ ID NO: 145 - RNA, 22 nt, Unknown
  source: /note=“Description of Unknown: miR 24-3p sequence”
uggcucaguucagcaggaacag

SEQ ID NO: 146 - RNA, 22 nt, Unknown
  source: /note=“Description of Unknown: miR 24-5p sequence”
ugccuacugagcugauaucagu

SEQ ID NO: 147 - RNA, 21 nt, Unknown
  source: /note=“Description of Unknown: miR 27-3p sequence”
uucacaguggcuaaguuccgc

SEQ ID NO: 148 - RNA, 22 nt, Unknown
  source: /note=“Description of Unknown: miR 27-5p sequence”
agggcuuagcugcuugugagca

SEQ ID NO: 149 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 150 - RNA, 119 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 151 - RNA, 142 nt, Artificial Sequence; identical to SEQ ID NO: 109
  source: /note=“Description of Artificial Sequence: Synthetic polynucleotide”

SEQ ID NO: 152 - skipped

SEQ ID NO: 153 - RNA, 23 nt, Unknown
  source: /note=“Description of Unknown: miR 155-5p sequence”
uuaaugcuaauugugauaggggu

SEQ ID NO: 154 - RNA, 23 nt, Unknown
  source: /note=“Description of Unknown: miR 155-5p binding site sequence”
accccuaucacaauuagcauuaa

SEQ ID NO: 155 - RNA, 188 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 156 - RNA, 140 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaguagugcuuucuacuuuaugguggucuuugaauaaagucugagugggcggc

SEQ ID NO: 157 - RNA, 182 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccuccuccccuuccugcacccguacccccaguagugcuuucuacuuuaugguggucuuugaauaaagucugagugggcggc

SEQ ID NO: 158 - RNA, 184 nt, Artificial Sequence
  source: /note=“Description of Artificial Sequence: Synthetic polynucleotide”
ugauaauagaguagugcuuucuacuuuauggcuggagccucgguggccaugcuucuugccccuugggccuccauaaaguaggaaacacuacauccccccagccccuccuccccuuccugcacccguacccccaguagugcuuucuacuuuaugguggucuuugaauaaagucugagugggcggc

SEQ ID NO: 159 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaccccuaucacaauuagcauuaaguggucuuugaauaaagucugagugggcggc

SEQ ID NO: 160 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 161 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 162 - RNA, 142 nt, Artificial Sequence; identical to SEQ ID NO: 104
  source: /note=“Description of Artificial Sequence: Synthetic polynucleotide”

SEQ ID NO: 163 - RNA, 142 nt, Artificial Sequence; identical to SEQ ID NO: 105
  source: /note=“Description of Artificial Sequence: Synthetic polynucleotide”

SEQ ID NO: 164 - RNA, 142 nt, Artificial Sequence
  source: /note=“Description of Artificial Sequence: Synthetic polynucleotide”
ugauaauaggcuggagccucgguggccaugcuucuugccccuugggccuccauaaaguaggaaacacuacauccccccagccccuccuccccuuccugcacccguacccccguggucuuugaauaaagucugagugggcggc

SEQ ID NO: 165 - RNA, 70 nt, Artificial Sequence
  source: /note=“Description of Artificial Sequence: Synthetic oligonucleotide”
gggaaauaagaguccauaaaguaggaaacacuacaagaaaagaagaguaagaagaaauauaagagccacc

SEQ ID NO: 166 - RNA, 70 nt, Artificial Sequence
  source: /note=“Description of Artificial Sequence: Synthetic oligonucleotide”
gggaaauaagagagaaaagaagaguaauccauaaaguaggaaacacuacagaagaaauauaagagccacc

SEQ ID NO: 167 - RNA, 70 nt, Artificial Sequence
  source: /note=“Description of Artificial Sequence: Synthetic oligonucleotide”
gggaaauaagagagaaaagaagaguaagaagaaauauaauccauaaaguaggaaacacuacagagccacc

SEQ ID NO: 168 - RNA, 23 nt, Unknown
  source: /note=“Description of Unknown: miR 155-5p binding site sequence”
accccuaucacaauuagcauuaa

SEQ ID NO: 169 - RNA, 181 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccucuccccuuccugcacccguacccccaguagugcuuucuacuuuaugguggucuuugaauaaagucugagugggcggc

SEQ ID NO: 170 - RNA, 142 nt, Artificial Sequence; identical to SEQ ID NO: 106
  source: /note=“Description of Artificial Sequence: Synthetic polynucleotide”

SEQ ID NO: 171 - RNA, 142 nt, Artificial Sequence; identical to SEQ ID NO: 107
  source: /note=“Description of Artificial Sequence: Synthetic polynucleotide”

SEQ ID NO: 172 - RNA, 142 nt, Artificial Sequence; identical to SEQ ID NO: 108
  source: /note=“Description of Artificial Sequence: Synthetic polynucleotide”

SEQ ID NO: 173 - RNA, 142 nt, Artificial Sequence; identical to SEQ ID NO: 110
  source: /note=“Description of Artificial Sequence: Synthetic polynucleotide”

SEQ ID NO: 174 - RNA, 164 nt, Artificial Sequence; identical to SEQ ID NO: 112
  source: /note=“Description of Artificial Sequence: Synthetic polynucleotide”

SEQ ID NO: 175 - RNA, 119 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 176 - skipped

SEQ ID NO: 177 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 178 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 179 - RNA, 142 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguacccccguggucuuugaauaaagucugagugggcggc

SEQ ID NO: 180 - RNA, 142 nt, Artificial Sequence
  source: /note=“Description of Artificial Sequence: Synthetic polynucleotide”
ugauaauaggcuggagccucgguggcuccauaaaguaggaaacacuacacuagcuucuugccccuugggccuccccccagccccuccuccccuuccugcacccguacccccguggucuuugaauaaagucugagugggcggc

SEQ ID NO: 181 - RNA, 142 nt, Artificial Sequence
  source: /note=“Description of Artificial Sequence: Synthetic polynucleotide”
ugauaauaggcuggagccucgguggccuagcuucuugccccuugggccuccauaaaguaggaaacacuacauccccccagccccuccuccccuuccugcacccguacccccguggucuuugaauaaagucugagugggcggc

SEQ ID NO: 182 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccaccccuaucacaauuagcauuaaguggucuuugaauaaagucugagugggcggc

SEQ ID NO: 183 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 184 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 185 - RNA, 47 nt, Unknown
  source: /note=“Description of Unknown: 5′ UTR sequence”
gggaaauaagagagaaaagaagaguaagaagaaauauaagagccacc

SEQ ID NO: 186 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 187 - RNA, 120 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 188 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 189 - RNA, 150 nt, Artificial Sequence
  source: /note=“Description of Artificial Sequence: Synthetic polynucleotide”
  misc_feature (1)..(150): /note=“This sequence may encompass 50-150 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 190 - RNA, 150 nt, Artificial Sequence; identical to SEQ ID NO: 189
  source: /note=“Description of Artificial Sequence: Synthetic polynucleotide”
  misc_feature (1)..(150): /note=“This sequence may encompass 75-150 nucleotides”

SEQ ID NO: 191 - RNA, 150 nt, Artificial Sequence; identical to SEQ ID NO: 189
  source: /note=“Description of Artificial Sequence: Synthetic polynucleotide”
  misc_feature (1)..(150): /note=“This sequence may encompass 85-150 nucleotides”

SEQ ID NO: 192 - RNA, 150 nt, Artificial Sequence; identical to SEQ ID NO: 189
  source: /note=“Description of Artificial Sequence: Synthetic polynucleotide”
  misc_feature (1)..(150): /note=“This sequence may encompass 90-150 nucleotides”

SEQ ID NO: 193 - RNA, 120 nt, Artificial Sequence; identical to SEQ ID NO: 187
  source: /note=“Description of Artificial Sequence: Synthetic polynucleotide”
  misc_feature (1)..(120): /note=“This sequence may encompass 90-120 nucleotides”

SEQ ID NO: 194 - RNA, 130 nt, Artificial Sequence
  source: /note=“Description of Artificial Sequence: Synthetic polynucleotide”
  misc_feature (1)..(130): /note=“This sequence may encompass 90-130 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0158458 A1
(43) Pub. Date: May 16, 2024

(54) MRNAS ENCODING IMMUNE MODULATING POLYPEPTIDES AND USES THEREOF

(71) Applicant: MODERNA TX, INC., Cambridge, MA (US)

(72) Inventors: Eric Yi-Chun Huang, Boston, MA (US); Jared Iacovelli, Waltham, MA (US); Seymour De Picciotto, Boston, MA (US); Sze-Wah Tse, Boston, MA (US); Laurie Kenney, Boston, MA (US)

(21) Appl. No.: 17/768,513

(22) PCT Filed: Oct. 15, 2020
(86) PCT No.: PCT/US2020/055844

§ 371 Date: Apr. 13, 2022

Related U.S. Application Data
(60) Provisional application No. 62/915,304, filed on Oct. 15, 2019.
(60) Provisional application No. 62/959,716, filed on Jan. 10, 2020.
(60) Provisional application No. 63/017,040, filed on Apr. 29, 2020.

(51) Int. Cl. C07K 14/55; A61K 47/69; A61P 37/02; C07K 14/535; C07K 16/28

(52) U.S. Cl. CPC C07K 14/55; A61K 47/6929; A61P 37/02; C07K 14/535; C07K 16/2818; C07K 2319/31

(57) ABSTRACT

The disclosure features lipid nanoparticle (LNP) compositions comprising immune modulating polypeptides and uses thereof. The LNP compositions of the present disclosure comprise mRNA therapeutics encoding immune modulating polypeptides, e.g., interleukin 2 (IL-2) and/or granulocyte macrophage colony stimulating factor (GM-CSF). The LNP compositions of the present disclosure can stimulate T regulatory cells, e.g., increase the level and/or activity of T regulatory cells in vivo or ex vivo.

### CROSS-REFERENCE TO RELATED APPLICATIONS

This application claims the benefit of U.S. Provisional Application No. 62/915,304, filed Oct. 15, 2019, U.S. Provisional Application No. 62/959,716, filed Jan. 10, 2020, and U.S. Provisional Application No. 63/017,040, filed Apr. 29, 2020. The contents of the aforesaid applications are hereby incorporated by reference in their entirety.

### SEQUENCE LISTING

This application contains a Sequence Listing which has been submitted electronically in ASCII format and is hereby incorporated by reference in its entirety. Said ASCII copy, created on Oct. 15, 2020, is named M2180-7001WO_SL.txt and is 268,894 bytes in size.

### BACKGROUND OF THE DISCLOSURE

Regulatory T cells (also known as T regulatory cells or T regs) are an important cell type in the maintenance of immune tolerance. The best-known type of regulatory T cells is a subset of CD4+ T cells defined by the expression of the transcription factor FOXP3. Animal studies have suggested that modulation of regulatory T cells may be useful for treating autoimmune disease or cancer. However, methods of stimulating and/or increasing the number of regulatory T cells in vivo remain under investigation. Therefore, there is an unmet need to develop therapies that can stimulate regulatory T cells and modulate immune responses.

### SUMMARY OF THE DISCLOSURE

The present disclosure provides, inter alia, lipid nanoparticle (LNP) compositions comprising immune modulating polypeptides and uses thereof. The LNP compositions of the present disclosure comprise mRNA therapeutics encoding immune modulating polypeptides, e.g., interleukin 2 (IL-2) and/or granulocyte macrophage colony stimulating factor (GM-CSF). In an aspect, the LNP compositions of the present disclosure can stimulate T regulatory cells, e.g., increase the level and/or activity of T regulatory cells in vivo or ex vivo. Also disclosed herein are methods of using an LNP composition comprising immune modulating polypeptides, e.g., IL-2 and/or GM-CSF, for treating and/or preventing a disease associated with an aberrant T regulatory cell function, or for inhibiting an immune response in a subject. Additional aspects of the disclosure are described in further detail below.

Accordingly, in an aspect, the disclosure provides a lipid nanoparticle (LNP) composition comprising a polynucleotide comprising an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables 1A, 2A or 4A.

In an embodiment, the IL-2 molecule comprises a naturally occurring IL-2 molecule, a fragment of a naturally occurring IL-2 molecule, or a variant thereof. In an embodiment, the IL-2 molecule comprises a variant of a naturally occurring IL-2 molecule (e.g., an IL-2 variant, e.g., as described herein), or a fragment thereof. In an embodiment, the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).

In another aspect, provided herein is a lipid nanoparticle (LNP) composition comprising a polynucleotide comprising an mRNA which encodes a GM-CSF molecule comprising an amino acid sequence having at least 85%, 90%, 95%, 96%, 97%, 98%, 99% or 100% identity to the amino acid sequence of a GM-CSF molecule provided in Table 3A or 3B.

In an embodiment, the GM-CSF molecule comprises a naturally occurring GM-CSF molecule, a fragment of a naturally occurring GM-CSF molecule, or a variant thereof. In an embodiment, the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220. In an embodiment, the GM-CSF molecule comprises the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.

In one aspect the invention features a lipid nanoparticle (LNP) composition, comprising: (a) a first polynucleotide encoding an IL-2 molecule; and (b) a second polynucleotide encoding a GM-CSF molecule, wherein (a) and (b) each comprise an mRNA.

In an embodiment, the first and second polynucleotides are formulated at an (a):(b) mass ratio of 10:1, 8:1, 6:1, 4:1, 3:1, 2:1, 1.5:1, or 1:1. In an embodiment, the first and second polynucleotides are formulated at an (a):(b) mass ratio of 1:1.5, 1:2, 1:3, 1:4, 1:6, 1:8, or 1:10. In an embodiment, the first and second polynucleotides are formulated at an (a):(b) mass ratio of 1:1.

In another aspect, disclosed herein is lipid nanoparticle (LNP) composition, for stimulating T regulatory cells, the LNP composition comprising: (a) a first polynucleotide encoding an IL-2 molecule; and (b) a second polynucleotide encoding a GM-CSF molecule, wherein (a) and (b) each comprise an mRNA.

In an embodiment of any of the LNP compositions disclosed herein, the IL-2 molecule comprises a naturally occurring IL-2 molecule, a fragment of a naturally occurring IL-2 molecule, or a variant thereof. In an embodiment, the IL-2 molecule comprises a variant of a naturally occurring IL-2 molecule (e.g., an IL-2 variant, e.g., as described herein), or a fragment thereof. In an embodiment, the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25). In an embodiment, the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.

In an embodiment of any of the LNP compositions disclosed herein, the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133. In an embodiment, the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.

In an embodiment of any of the LNP compositions disclosed herein, the IL-2 variant comprises a mutation, e.g., substitution, at position 88 of the IL-2 polypeptide sequence, e.g., an N88D substitution.

In an embodiment of any of the LNP compositions disclosed herein, the IL-2 variant comprises a mutation, e.g., substitution, at position 91 of the IL-2 polypeptide sequence, e.g., a V91K substitution.

In an embodiment of any of the LNP compositions disclosed herein, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and position 88 of the IL-2 polypeptide sequence, e.g., an N88D substitution.

In an embodiment of any of the LNP compositions disclosed herein, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and position 91 of the IL-2 polypeptide sequence, e.g., a V91K substitution.

In an embodiment of any of the LNP compositions disclosed herein, the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35. In an embodiment, the IL-2 molecule comprises the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35. In an embodiment, the IL-2 molecule comprises the amino acid sequence of SEQ ID NO: 1. In an embodiment, the IL-2 molecule comprises the amino acid sequence of SEQ ID NO: 2. In an embodiment, the IL-2 molecule comprises the amino acid sequence of SEQ ID NO: 3. In an embodiment, the IL-2 molecule comprises the amino acid sequence of SEQ ID NO: 4. In an embodiment, the IL-2 molecule comprises the amino acid sequence of SEQ ID NO: 5. In an embodiment, the IL-2 molecule comprises the amino acid sequence of SEQ ID NO: 6. In an embodiment, the IL-2 molecule comprises the amino acid sequence of SEQ ID NO: 30. In an embodiment, the IL-2 molecule comprises the amino acid sequence of SEQ ID NO: 31. In an embodiment, the IL-2 molecule comprises the amino acid sequence of SEQ ID NO: 32. In an embodiment, the IL-2 molecule comprises the amino acid sequence of SEQ ID NO: 33. In an embodiment, the IL-2 molecule comprises the amino acid sequence of SEQ ID NO: 34. In an embodiment, the IL-2 molecule comprises the amino acid sequence of SEQ ID NO: 35.

In an embodiment of any of the LNP compositions disclosed herein, the polynucleotide encoding an IL-2 molecule (e.g., first polynucleotide encoding an IL-2 molecule) comprises a nucleotide sequence having at least 85%, 90%, 95%, 96%, 97%, 98%, 99% or 100% identity to the sequence of SEQ ID NO: 7. In an embodiment, the polynucleotide encoding an IL-2 molecule (e.g., first polynucleotide encoding an IL-2 molecule) comprises the nucleotide sequence of SEQ ID NO: 7.

In an embodiment of any of the LNP compositions disclosed herein, the LNP composition comprises a polynucleotide (e.g., mRNA), e.g., a first polynucleotide, encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to the amino acid sequence of SEQ ID NO: 11. In an embodiment, the IL-2 molecule comprises the amino acid sequence of SEQ ID NO: 11. In an embodiment, the first polynucleotide (e.g., mRNA) encoding the IL-2 molecule comprises a nucleotide sequence having at least 85%, 90%, 95%, 96%, 97%, 98%, 99% or 100% identity to the sequence of SEQ ID NO: 25. In an embodiment, the polynucleotide (e.g., mRNA), e.g., first polynucleotide, encoding the IL-2 molecule comprises the nucleotide sequence of SEQ ID NO: 25. In an embodiment, the first polynucleotide (e.g., mRNA) encoding the IL-2 molecule comprises the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In an embodiment of any of the LNP compositions disclosed herein, the LNP composition comprises a polynucleotide (e.g., mRNA), e.g., a first polynucleotide, encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to the amino acid sequence of SEQ ID NO: 11. In an embodiment, the IL-2 molecule comprises the amino acid sequence of SEQ ID NO: 11. In an embodiment, the first polynucleotide (e.g., mRNA) encoding the IL-2 molecule comprises a nucleotide sequence having at least 85%, 90%, 95%, 96%, 97%, 98%, 99% or 100% identity to the sequence of SEQ ID NO: 36. In an embodiment, the polynucleotide (e.g., mRNA), e.g., first polynucleotide, encoding the IL-2 molecule comprises the nucleotide sequence of SEQ ID NO: 36. In an embodiment, the first polynucleotide (e.g., mRNA) encoding the IL-2 molecule comprises the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

In an embodiment of any of the LNP compositions disclosed herein, the IL-2 and/or the GMCSF molecule comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the albumin is HSA comprising an amino acid sequence having at least 85%, 90%, 95%, 96%, 97%, 98%, 99% or 100% identity to the amino acid sequence of SEQ ID NO: 8. In an embodiment, the albumin is HSA comprising the amino acid sequence of SEQ ID NO: 8.

In an embodiment of any of the LNP compositions disclosed herein, the IL-2 molecule comprising human serum albumin (HSA) comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13. In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 without the leader sequence. In an embodiment, the IL-2 molecule comprising human serum albumin (HSA) comprises the amino acid sequence of SEQ ID NO: 9, SEQ ID NO: 10, SEQ ID NO: 11, SEQ ID NO: 12 or SEQ ID NO: 13. In an embodiment, the IL-2 molecule comprising human serum albumin (HSA) comprises the amino acid sequence of SEQ ID NO: 9, SEQ ID NO: 10, SEQ ID NO: 11, SEQ ID NO: 12 or SEQ ID NO: 13 without the leader sequence. In an embodiment, the IL-2 molecule comprising human serum albumin (HSA) comprises the amino acid sequence of SEQ ID NO: 9. In an embodiment, the IL-2 molecule comprising human serum albumin (HSA) comprises the amino acid sequence of SEQ ID NO: 9 without the leader sequence. In an embodiment, the IL-2 molecule comprising human serum albumin (HSA) comprises the amino acid sequence of SEQ ID NO: 10. In an embodiment, the IL-2 molecule comprising human serum albumin (HSA) comprises the amino acid sequence of SEQ ID NO: 10 without the leader sequence. In an embodiment, the IL-2 molecule comprising human serum albumin (HSA) comprises the amino acid sequence of SEQ ID NO: 11. In an embodiment, the IL-2 molecule comprising human serum albumin (HSA) comprises the amino acid sequence of SEQ ID NO: 11 without the leader sequence. In an embodiment, the IL-2 molecule comprising human serum albumin (HSA) comprises the amino acid sequence of SEQ ID NO: 12. In an embodiment, the IL-2 molecule comprising human serum albumin (HSA) comprises the amino acid sequence of SEQ ID NO: 12 without the leader sequence. In an embodiment, the IL-2 molecule comprising human serum albumin (HSA) comprises the amino acid sequence of SEQ ID NO: 13. In an embodiment, the IL-2 molecule comprising human serum albumin (HSA) comprises the amino acid sequence of SEQ ID NO: 13 without the leader sequence.

In an embodiment, the polynucleotide encoding the IL-2 molecule which comprises human serum albumin (HSA), comprises a nucleotide sequence having at least 85%, 90%, 95%, 96%, 97%, 98%, 99% or 100% identity to the sequence of SEQ ID NO: 25. In an embodiment, the polynucleotide encoding the IL-2 molecule which comprises human serum albumin (HSA), comprises the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In an embodiment, the polynucleotide encoding the IL-2 molecule which comprises human serum albumin (HSA), comprises a nucleotide sequence having at least 85%, 90%, 95%, 96%, 97%, 98%, 99% or 100% identity to the sequence of SEQ ID NO: 36. In an embodiment, the polynucleotide encoding the IL-2 molecule which comprises human serum albumin (HSA), comprises the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

In an embodiment of any of the LNP compositions disclosed herein, the IL-2 molecule further comprises a targeting moiety, e.g., a T regulatory cell targeting moiety or a tissue-specific targeting moiety.

In an embodiment of any of the LNP compositions disclosed herein, the IL-2 molecule further comprises a T regulatory cell targeting moiety. In an embodiment, the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof. In an embodiment, the T regulatory cell targeting moiety binds to a molecule present on a T regulatory cell. In an embodiment, the T regulatory cell targeting moiety comprises an antibody molecule that binds to CTLA-4, GITR, TLR8, or Nrp1.

In an embodiment of any of the LNP compositions disclosed herein, the T regulatory cell targeting moiety comprises an antibody molecule that binds to CTLA-4. In an embodiment, the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17. In an embodiment, the targeting moiety comprises the amino acid sequence of SEQ ID NO: 17.

In an embodiment of any of the LNP compositions disclosed herein, the IL-2 molecule comprising the targeting moiety comprises an amino acid sequence having at least 85%, 90%, 95%, 96%, 97%, 98%, 99% or 100% identity to the amino acid sequence of SEQ ID NO: 17. In an embodiment, the IL-2 molecule comprising the targeting moiety comprises the amino acid sequence of SEQ ID NO: 17.

In an embodiment of any of the LNP compositions disclosed herein, the IL-2 molecule comprising the targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20. In an embodiment, the IL-2 molecule comprising the targeting moiety comprises the amino acid sequence of SEQ ID NO: 18, SEQ ID NO: 19, or SEQ ID NO: 20.

In an embodiment of any of the LNP compositions disclosed herein, the IL-2 molecule further comprises a tissue targeting moiety. In an embodiment, the tissue-specific targeting moiety binds to ROS-CII, EDA, EDB, TnC A1, SyETP, GLUT-2, GD2, FAP, VCAM or MADCAM.

In an embodiment of any of the LNP compositions disclosed herein, the GM-CSF molecule comprises a naturally occurring GM-CSF molecule, a fragment of a naturally occurring GM-CSF molecule, or a variant thereof. In an embodiment, the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220. In an embodiment, the GM-CSF molecule comprises the amino acid sequence of SEQ ID NO: 14. In an embodiment, the GM-CSF molecule comprises the amino acid sequence of SEQ ID NO: 188. In an embodiment, the GM-CSF molecule comprises the amino acid sequence of SEQ ID NO: 39. In an embodiment, the GM-CSF molecule comprises the amino acid sequence of SEQ ID NO: 41. In an embodiment, the GM-CSF molecule comprises the amino acid sequence of SEQ ID NO: 43. In an embodiment, the GM-CSF molecule comprises the amino acid sequence of SEQ ID NO: 16. In an embodiment, the GM-CSF molecule comprises the amino acid sequence of SEQ ID NO: 200. In an embodiment, the GM-CSF molecule comprises the amino acid sequence of SEQ ID NO: 205. In an embodiment, the GM-CSF molecule comprises the amino acid sequence of SEQ ID NO: 210. In an embodiment, the GM-CSF molecule comprises the amino acid sequence of SEQ ID NO: 215. In an embodiment, the GM-CSF molecule comprises the amino acid sequence of SEQ ID NO: 220.

In an embodiment of any of the LNP compositions disclosed herein, a GM-CSF molecule comprises a nucleotide sequence having at least 85%, 90%, 95%, 96%, 97%, 98%, 99% or 100% identity to the nucleic acid sequence of SEQ ID NO: 15. In an embodiment, the GM-CSF molecule comprises the nucleic acid sequence of SEQ ID NO: 15. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 14.

In an embodiment, the polynucleotide, e.g., second polynucleotide, encoding the GM-CSF molecule comprises a nucleotide sequence having at least 85%, 90%, 95%, 96%, 97%, 98%, 99% or 100% identity to the nucleic acid sequence of SEQ ID NO: 38. In an embodiment, the polynucleotide, e.g., second polynucleotide encoding the GM-CSF molecule comprises the nucleotide sequence of SEQ ID NO: 38. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 188.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 40. In an embodiment, the polynucleotide, e.g., second polynucleotide encoding the GM-CSF molecule comprises the nucleotide sequence of SEQ ID NO: 40. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 39.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 42. In an embodiment, the polynucleotide, e.g., second polynucleotide encoding the GM-CSF molecule comprises the nucleotide sequence of SEQ ID NO: 42. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 41.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 44. In an embodiment, the polynucleotide, e.g., second polynucleotide encoding the GM-CSF molecule comprises the nucleotide sequence of SEQ ID NO: 44. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 43.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 201. In an embodiment, the polynucleotide, e.g., second polynucleotide encoding the GM-CSF molecule comprises the nucleotide sequence of SEQ ID NO: 201. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 200.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 206. In an embodiment, the polynucleotide, e.g., second polynucleotide encoding the GM-CSF molecule comprises the nucleotide sequence of SEQ ID NO: 206. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 205.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 211. In an embodiment, the polynucleotide, e.g., second polynucleotide encoding the GM-CSF molecule comprises the nucleotide sequence of SEQ ID NO: 211. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 210.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 216. In an embodiment, the polynucleotide, e.g., second polynucleotide encoding the GM-CSF molecule comprises the nucleotide sequence of SEQ ID NO: 216. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 215.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 221. In an embodiment, the polynucleotide, e.g., second polynucleotide encoding the GM-CSF molecule comprises the nucleotide sequence of SEQ ID NO: 221. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 219. In an embodiment, the polynucleotide, e.g., second polynucleotide encoding the GM-CSF molecule comprises the nucleotide sequence of SEQ ID NO: 219. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 224. In an embodiment, the polynucleotide, e.g., second polynucleotide encoding the GM-CSF molecule comprises the nucleotide sequence of SEQ ID NO: 224. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment of any of the LNP compositions disclosed herein, the GM-CSF molecule comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the albumin is HSA comprising an amino acid sequence having at least 85%, 90%, 95%, 96%, 97%, 98%, 99% or 100% identity to the amino acid sequence of SEQ ID NO:8. In an embodiment, the albumin is HSA comprising the amino acid sequence of SEQ ID NO:8.

In an embodiment of any of the LNP compositions disclosed herein, the GM-CSF molecule further comprises a targeting moiety, e.g., a dendritic cell targeting moiety, or a tissue-specific targeting moiety. In an embodiment, the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

In yet another aspect, the disclosure provides a pharmaceutical composition comprising an LNP disclosed herein. In one embodiment, the pharmaceutical composition is formulated for subcutaneous administration.

In an embodiment, the pharmaceutical composition comprises a pharmaceutically acceptable carrier or excipient.

While the LNPs comprising polynucleotides encoding IL-2 or GMCSF can be administered alone as monotherapies, in another aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, in the treatment and/or prophylaxis of a disease associated with an aberrant T regulatory cell function in a subject.

In a related aspect, provided herein is a method of treating and/or prophylaxis of a disease associated with an aberrant T regulatory cell function in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule. It will be understood that first and second in this context do not imply a particular order of administration, as set forth in more detail below.

In another aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for inhibiting an immune response in a subject.

In a related aspect, provided herein is method of inhibiting an immune response in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

In an aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for stimulating T regulatory cells in a subject.

In a related aspect, provided herein is a method of stimulating T regulatory cells in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

In another aspect, the disclosure provides a composition comprising a lipid nanoparticle (LNP) comprising a first polynucleotide encoding an IL-2 molecule and a second polynucleotide encoding a GM-CSF molecule for use, in the treatment of a disease associated with an aberrant T regulatory cell function in a subject.

In a related aspect, provided herein is a method of treating and/or prophylaxis of a disease associated with an aberrant T regulatory cell function in a subject, comprising administering to the subject an effective amount of a lipid nanoparticle (LNP) comprising a first polynucleotide encoding an IL-2 molecule and a second polynucleotide encoding a GM-CSF molecule.

In an embodiment, prior to the administration of the LNP comprising the first polynucleotide encoding the IL-2 molecule and the second polynucleotide encoding the GM-CSF molecule, a different LNP comprising a third polynucleotide encoding a GM-CSF molecule is administered to the subject.

In an embodiment, the LNP comprising a third polynucleotide encoding the GM-CSF molecule does not comprise a polynucleotide encoding an IL-2 molecule.

In an embodiment, the second polynucleotide encoding GM-CSF and the third polynucleotide encoding GM-CSF comprise the same or substantially the same polynucleotide sequence.

In an embodiment, the different LNP comprising a third polynucleotide encoding a GM-CSF molecule is administered at least 1 day, 2 days, 3 days, 4 days, 5 days, 6 days, 7 days, 8 days, 9 days, or 10 days (e.g., 7 days), prior to the administration of the LNP comprising a first polynucleotide encoding an IL-2 molecule and a second polynucleotide encoding a GM-CSF molecule.

In an embodiment, the different LNP comprising a third polynucleotide encoding a GM-CSF molecule is administered at least 1 week, 2 weeks, 3 weeks, 4 weeks, 5 weeks, or 6 weeks prior to the administration of the LNP comprising a first polynucleotide encoding an IL-2 molecule and a second polynucleotide encoding a GM-CSF molecule.

In an embodiment, the LNP comprising the first polynucleotide encoding an IL-2 molecule and a second polynucleotide encoding a GM-CSF molecule, and the LNP comprising a third polynucleotide encoding a GM-CSF molecule are administered at a dose disclosed herein.

In an embodiment, the dose, e.g., effective dose, of the GM-CSF molecule in the LNP comprising the third polynucleotide encoding GM-CSF is at least 10%, 20%, 30%, 40%, 50%, 60%, 70%, 80% or 95% lesser than the dose, e.g., effective dose, of the GM-CSF molecule in the LNP comprising the first and second polynucleotides. In an embodiment, the dose, e.g., effective dose, of the first polynucleotide encoding the IL-2 molecule in the lipid nanoparticle is at least 10%, 20%, 30%, 40%, 50%, 60%, 70%, 80% or 95% lesser than the dose of a naturally occurring, or recombinant IL-2, e.g., in an otherwise similar LNP.

In an embodiment of any of the compositions or methods provided herein, one or more LNP compositions described herein is administered subcutaneously.

In an embodiment of any of the compositions or methods provided herein, one or more LNP compositions described herein is administered at a dosing interval. In an embodiment, a dosing interval comprises repeated administration (e.g., repeated dosing) of one or more LNP compositions described herein. In an embodiment, in a dosing interval comprising repeated dosing, an LNP composition is administered repeatedly, e.g., the same LNP composition is administered repeatedly. In an embodiment, in a dosing interval comprising repeated dosing, one or more doses of a first LNP composition is administered followed by one or more doses of a different LNP compositions. In an embodiment, in a dosing interval comprising repeated dosing, one or more doses of a first LNP composition is administered followed by one or more doses of the first LNP composition in combination with a different LNP composition.

In an embodiment, repeated dosing comprises administration of an LNP composition about 1, 2, 3, 4, 5, 6, 7, 8, 9, 10, 15, 20, 30, 40 or 50 times, or about 1-50 times, 1-40 times, 1-30 times, 1-25 times, 1-20 times, 1-15 times, or 1-10 times. In an embodiment, a dosing interval comprising repeated administration can be performed over a period of time, e.g., at least 5-20 days, 5-19 days, 5-18 days, 5-17 days, 5-16 days, 5-15 days, 5-14 days, 5-13 days, 5-12 days, 5-11 days, 5-10 days, 5-9 days, 5-8 days, 5-7 days, 5-6 days, 6-20 days, 7-20 days, 8-20 days, 9-20 days, 10-20 days, 11-20 days, 12-20 days, 13-20 days, 14-20 days, 15-20 days, 16-20 days, 17-20 days, 18-20 days, or 19-20 days, e.g., 7-14 days. In an embodiment, a dosing interval comprising repeated administration can be performed over a period of time, e.g., over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years or 5 years.

In an embodiment, a dosing interval (e.g., repeated dosing) comprises an initial dose of an LNP composition and one or more subsequent doses of an LNP composition, e.g., the same or different LNP composition.

In an embodiment, an LNP composition described herein can be administered in combination with an additional LNP composition, e.g., a same or different LNP composition. In an embodiment, the LNP compositions can be administered simultaneously, substantially simultaneously, or sequentially. In an embodiment, the order of administration can be reversed.

In an aspect, the disclosure provides, a lipid nanoparticle comprising a polynucleotide encoding a molecule that stimulates T regulatory cells (e.g., an IL-2 molecule) for use, in the treatment of a disease associated with an aberrant T regulatory cell function in a subject.

In another aspect, provided herein is a method of treating and/or prophylaxis of a disease associated with an aberrant T regulatory cell function in a subject, comprising administering to the subject an effective amount of a lipid nanoparticle comprising a polynucleotide encoding a molecule that stimulates T regulatory cells (e.g., an IL-2 molecule).

In an embodiment, the method or composition for use further comprises administration of a lipid nanoparticle comprising a polynucleotide encoding a GM-CSF molecule.

In an embodiment, the LNP comprising the polynucleotide encoding the IL-2 molecule and the LNP comprising the polynucleotide encoding the GM-CSF molecule can be administered sequentially. In an embodiment, the LNP composition comprising the polynucleotide encoding the GM-CSF molecule is administered first and the LNP composition comprising the polynucleotide encoding the IL-2 molecule is administered second. In an embodiment, the LNP composition comprising the polynucleotide encoding the GM-CSF molecule is administered second and the LNP composition comprising the polynucleotide encoding the IL-2 molecule is administered first. In an embodiment, the LNP composition comprising the polynucleotide encoding the GM-CSF molecule is administered after administration of the LNP composition comprising the polynucleotide encoding the IL-2 molecule. In an embodiment, the LNP composition comprising the polynucleotide encoding the IL-2 molecule is administered after administration of the LNP composition comprising the polynucleotide encoding the GM-CSF molecule.

In an embodiment, the LNP composition comprising the polynucleotide encoding the GM-CSF molecule and the LNP composition comprising the polynucleotide encoding the IL-2 molecule are administered simultaneously, e.g., substantially simultaneously.

In an embodiment, the LNP composition comprising the polynucleotide encoding the GM-CSF molecule and the LNP composition comprising the polynucleotide encoding the IL-2 molecule are in the same composition. In an embodiment, the LNP composition comprising the polynucleotide encoding the GM-CSF molecule and the LNP composition comprising the polynucleotide encoding the IL-2 molecule are in different compositions.

In an embodiment, the molecule that stimulates T regulatory cells comprises an IL-2 molecule, or a molecule that binds to a receptor present on T regulatory cells.

In yet another aspect, the disclosure provides a lipid nanoparticle (LNP) comprising a polynucleotide encoding a molecule that stimulates dendritic cells (e.g., a GM-CSF molecule) for use, in the treatment of a disease associated with an aberrant T regulatory cell function in a subject.

In a related aspect, provided herein is a method of treating and/or prophylaxis of a disease associated with an aberrant T regulatory cell function in a subject, comprising administering to a subject an effective amount of a lipid nanoparticle comprising a polynucleotide encoding molecule that stimulates dendritic cells (e.g., a GM-CSF molecule).

In an embodiment, the method or composition for use further comprises administration of a lipid nanoparticle comprising a polynucleotide encoding an IL-2 molecule.

In an embodiment, the LNP comprising the polynucleotide encoding the IL-2 molecule and the LNP comprising the polynucleotide encoding the GM-CSF molecule can be administered sequentially. In an embodiment, the LNP composition comprising the polynucleotide encoding the GM-CSF molecule is administered first and the LNP composition comprising the polynucleotide encoding the IL-2 molecule is administered second. In an embodiment, the LNP composition comprising the polynucleotide encoding the GM-CSF molecule is administered second and the LNP composition comprising the polynucleotide encoding the IL-2 molecule is administered first. In an embodiment, the LNP composition comprising the polynucleotide encoding the GM-CSF molecule is administered after administration of the LNP composition comprising the polynucleotide encoding the IL-2 molecule. In an embodiment, the LNP composition comprising the polynucleotide encoding the IL-2 molecule is administered after administration of the LNP composition comprising the polynucleotide encoding the GM-CSF molecule.

In an embodiment, the LNP composition comprising the polynucleotide encoding the GM-CSF molecule and the LNP composition comprising the polynucleotide encoding the IL-2 molecule are administered simultaneously, e.g., substantially simultaneously.

In an embodiment, the LNP composition comprising the polynucleotide encoding the GM-CSF molecule and the LNP composition comprising the polynucleotide encoding the IL-2 molecule are in the same composition. In an embodiment, the LNP composition comprising the polynucleotide encoding the GM-CSF molecule and the LNP composition comprising the polynucleotide encoding the IL-2 molecule are in different compositions.

In an embodiment, the molecule that stimulates dendritic cells comprises a molecule that stimulates, e.g., increases, the expression and/or level of TNF alpha, IL-10, CCL-2 and/or nitric oxide in dendritic cells.

In an embodiment, the molecule that stimulates dendritic cells comprises a GM-CSF molecule, e.g., as described herein.

In an embodiment, the molecule that stimulates dendritic cells results in an increased level and/or activity of CD11b+ or CD11c+ dendritic cells.

In an embodiment, administration of the LNP comprising the polynucleotide encoding the GM-CSF molecule results in a modulation of dendritic cell activity and/or modulation of expression and/or activity of myeloid cells in a sample from the subject. In an embodiment, the sample has an increase in, e.g., increased number or proportion of, dendritic cells expressing CD11b and/or CD11c. In an embodiment, the increase in DCs expressing CD11c (CD11c+ DCs) is at least 1.2 to 20 fold (e.g., at least 1.2, 1.5, 2, 3, 4, 5, 10, 15, or 20 fold), e.g., as compared to an otherwise similar sample not contacted with the LNP comprising the GM-CSF molecule, or contacted with a different LNP.

In an embodiment, the sample has an increase in, e.g., increased number or proportion of, myeloid cells expressing CD11b, e.g., as compared to an otherwise similar sample not contacted with the LNP comprising the GM-CSF molecule, or contacted with a different LNP.

Surprisingly, as shown herein, the administration of LNP comprising an mRNA encoding a GM-CSF molecule (e.g., a GM-CSF molecule described herein) or an mRNA encoding an IL-2 molecule (e.g., an IL-2 molecule described herein) as a monotherapy, or in combination, produces beneficial effects in vivo after subcutaneous administration.

In an embodiment of any of the methods or compositions for use disclosed herein, the IL-2 molecule comprises a naturally occurring IL-2 molecule, a fragment of a naturally occurring IL-2 molecule, or a variant thereof. In an embodiment, the IL-2 molecule comprises a variant of a naturally occurring IL-2 molecule (e.g., an IL-2 variant, e.g., as described herein), or a fragment thereof. In an embodiment, the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25). In an embodiment, the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.

In an embodiment of any of the methods or compositions for use disclosed herein, the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133. In an embodiment, the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.

In an embodiment of any of the methods or compositions for use disclosed herein, the IL-2 variant comprises a mutation, e.g., substitution, at position 88 of the IL-2 polypeptide sequence, e.g., an N88D substitution.

In an embodiment of any of the methods or compositions for use disclosed herein, the IL-2 variant comprises a mutation, e.g., substitution, at position 91 of the IL-2 polypeptide sequence, e.g., a V91K substitution.

In an embodiment of any of the methods or compositions for use disclosed herein, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and position 88 of the IL-2 polypeptide sequence, e.g., an N88D substitution.

In an embodiment of any of the methods or compositions for use disclosed herein, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and position 91 of the IL-2 polypeptide sequence, e.g., a V91K substitution.

In an embodiment of any of the methods or compositions for use disclosed herein, the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35. In an embodiment, the IL-2 molecule comprises the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35. In an embodiment, the IL-2 molecule comprises the amino acid sequence of SEQ ID NO: 1. In an embodiment, the IL-2 molecule comprises the amino acid sequence of SEQ ID NO: 2. In an embodiment, the IL-2 molecule comprises the amino acid sequence of SEQ ID NO: 3. In an embodiment, the IL-2 molecule comprises the amino acid sequence of SEQ ID NO: 4. In an embodiment, the IL-2 molecule comprises the amino acid sequence of SEQ ID NO: 5. In an embodiment, the IL-2 molecule comprises the amino acid sequence of SEQ ID NO: 6. In an embodiment, the IL-2 molecule comprises the amino acid sequence of SEQ ID NO: 30. In an embodiment, the IL-2 molecule comprises the amino acid sequence of SEQ ID NO: 31. In an embodiment, the IL-2 molecule comprises the amino acid sequence of SEQ ID NO: 32. In an embodiment, the IL-2 molecule comprises the amino acid sequence of SEQ ID NO: 33. In an embodiment, the IL-2 molecule comprises the amino acid sequence of SEQ ID NO: 34. In an embodiment, the IL-2 molecule comprises the amino acid sequence of SEQ ID NO: 35.

In an embodiment of any of the methods or compositions for use disclosed herein, the first polynucleotide encoding an IL-2 molecule comprises a nucleotide sequence having at least 85%, 90%, 95%, 96%, 97%, 98%, 99% or 100% identity to the sequence of SEQ ID NO: 7. In an embodiment, the first polynucleotide encoding an IL-2 molecule comprises the nucleotide sequence of SEQ ID NO: 7.

In an embodiment of any of the methods or compositions for use disclosed herein, the IL-2 molecule comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the albumin is HSA comprising an amino acid sequence having at least 85%, 90%, 95%, 96%, 97%, 98%, 99% or 100% identity to the amino acid sequence of SEQ ID NO: 8. In an embodiment, the albumin is HSA comprising the amino acid sequence of SEQ ID NO: 8.

In an embodiment of any of the methods or compositions for use disclosed herein, the IL-2 molecule comprising human serum albumin (HSA) comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without the leader sequence. In an embodiment, the IL-2 molecule comprising human serum albumin (HSA) comprises the amino acid sequence of SEQ ID NO: 9, SEQ ID NO: 10, SEQ ID NO: 11, SEQ ID NO: 12 or SEQ ID NO: 13 with or without the leader sequence. In an embodiment, the IL-2 molecule comprising human serum albumin (HSA) comprises the amino acid sequence of SEQ ID NO: 9. In an embodiment, the IL-2 molecule comprising human serum albumin (HSA) comprises the amino acid sequence of SEQ ID NO: 10. In an embodiment, the IL-2 molecule comprising human serum albumin (HSA) comprises the amino acid sequence of SEQ ID NO: 11. In an embodiment, the IL-2 molecule comprising human serum albumin (HSA) comprises the amino acid sequence of SEQ ID NO: 12. In an embodiment, the IL-2 molecule comprising human serum albumin (HSA) comprises the amino acid sequence of SEQ ID NO: 13.

In an embodiment of any of the methods or compositions for use disclosed herein, the polynucleotide encoding the IL-2 molecule comprising human serum albumin (HSA) comprises a nucleotide sequence having at least 85%, 90%, 95%, 96%, 97%, 98%, 99% or 100% identity to the sequence of SEQ ID NO: 25. In an embodiment, the polynucleotide encoding the IL-2 molecule comprising human serum albumin (HSA) comprises the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In an embodiment of any of the methods or compositions for use disclosed herein, the polynucleotide encoding the IL-2 molecule comprising human serum albumin (HSA) comprises a nucleotide sequence having at least 85%, 90%, 95%, 96%, 97%, 98%, 99% or 100% identity to the sequence of SEQ ID NO: 36. In an embodiment, the polynucleotide encoding the IL-2 molecule comprising human serum albumin (HSA) comprises the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

In an embodiment of any of the methods or compositions for use disclosed herein, the IL-2 molecule further comprises a targeting moiety, e.g., a T regulatory cell targeting moiety or a tissue-specific targeting moiety.

In an embodiment of any of the methods or compositions for use disclosed herein, the IL-2 molecule further comprises a T regulatory cell targeting moiety. In an embodiment, the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof. In an embodiment, the T regulatory cell targeting moiety binds to a molecule present on a T regulatory cell. In an embodiment, the T regulatory cell targeting moiety comprises an antibody molecule that binds to CTLA-4, GITR, TLR8, or Nrp1.

In an embodiment of any of the methods or compositions for use disclosed herein, the T regulatory cell targeting moiety comprises an antibody molecule that binds to CTLA-4. In an embodiment, the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17. In an embodiment, the CTLA4 targeting moiety comprises the amino acid sequence of SEQ ID NO: 17.

In an embodiment of any of the methods or compositions for use disclosed herein, the IL-2 molecule comprising the CTLA-4 targeting moiety comprises an amino acid sequence having at least 85%, 90%, 95%, 96%, 97%, 98%, 99% or 100% identity to the amino acid sequence of SEQ ID NO: 17. In an embodiment, the IL-2 molecule comprising the CTLA-4 targeting moiety comprises the amino acid sequence of SEQ ID NO: 17.

In an embodiment of any of the methods or compositions for use disclosed herein, the IL-2 molecule comprising the CTLA-4 targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20. In an embodiment, the IL-2 molecule comprising the CTLA-4 targeting moiety comprises the amino acid sequence of SEQ ID NO: 18, SEQ ID NO: 19, or SEQ ID NO: 20. In an embodiment, the IL-2 molecule comprising the CTLA-4 targeting moiety is encoded by the a nucleotide sequence having at least 85%, 90%, 95%, 96%, 97%, 98%, 99% or 100% identity to the nucleic acid sequence of SEQ ID NO: 21, SEQ ID NO: 22 or SEQ ID NO: 23.

In an embodiment of any of the methods or compositions for use disclosed herein, the IL-2 molecule further comprises a tissue targeting moiety. In an embodiment, the tissue-specific targeting moiety binds to ROS-CII, EDA, EDB, TnC A1, SyETP, GLUT-2, GD2, FAP, VCAM or MADCAM.

In an embodiment of any of the methods or compositions for use disclosed herein, the GM-CSF molecule comprises a naturally occurring GM-CSF molecule, a fragment of a naturally occurring GM-CSF molecule, or a variant thereof. In an embodiment, the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41 or SEQ ID NO: 43. In an embodiment, the GM-CSF molecule comprises the amino acid sequence of SEQ ID NO: 14. In an embodiment, the GM-CSF molecule comprises the amino acid sequence of SEQ ID NO: 188. In an embodiment, the GM-CSF molecule comprises the amino acid sequence of SEQ ID NO: 39. In an embodiment, the GM-CSF molecule comprises the amino acid sequence of SEQ ID NO: 41. In an embodiment, the GM-CSF molecule comprises the amino acid sequence of SEQ ID NO: 43. In an embodiment, the GM-CSF molecule comprises the amino acid sequence of SEQ ID NO: 16. In an embodiment, the GM-CSF molecule comprises the amino acid sequence of SEQ ID NO: 200. In an embodiment, the GM-CSF molecule comprises the amino acid sequence of SEQ ID NO: 205. In an embodiment, the GM-CSF molecule comprises the amino acid sequence of SEQ ID NO: 210. In an embodiment, the GM-CSF molecule comprises the amino acid sequence of SEQ ID NO: 215. In an embodiment, the GM-CSF molecule comprises the amino acid sequence of SEQ ID NO: 220.

In an embodiment of any of the methods or compositions for use disclosed herein, the second polynucleotide encoding a GM-CSF molecule comprises a nucleotide sequence having at least 85%, 90%, 95%, 96%, 97%, 98%, 99% or 100% identity to the nucleic acid sequence of SEQ ID NO: 15. In an embodiment, the second polynucleotide encoding a GM-CSF molecule comprises the nucleic acid sequence of SEQ ID NO: 15. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 14.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 38. In an embodiment, the polynucleotide, e.g., second polynucleotide encoding the GM-CSF molecule comprises the nucleotide sequence of SEQ ID NO: 38. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 188.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 40. In an embodiment, the polynucleotide, e.g., second polynucleotide encoding the GM-CSF molecule comprises the nucleotide sequence of SEQ ID NO: 40. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 39.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 42. In an embodiment, the polynucleotide, e.g., second polynucleotide encoding the GM-CSF molecule comprises the nucleotide sequence of SEQ ID NO: 42. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 41.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 44. In an embodiment, the polynucleotide, e.g., second polynucleotide encoding the GM-CSF molecule comprises the nucleotide sequence of SEQ ID NO: 44. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 43.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 201. In an embodiment, the polynucleotide, e.g., second polynucleotide encoding the GM-CSF molecule comprises the nucleotide sequence of SEQ ID NO: 201. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 200.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 206. In an embodiment, the polynucleotide, e.g., second polynucleotide encoding the GM-CSF molecule comprises the nucleotide sequence of SEQ ID NO: 206. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 205.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 211. In an embodiment, the polynucleotide, e.g., second polynucleotide encoding the GM-CSF molecule comprises the nucleotide sequence of SEQ ID NO: 211. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 210.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 216. In an embodiment, the polynucleotide, e.g., second polynucleotide encoding the GM-CSF molecule comprises the nucleotide sequence of SEQ ID NO: 216. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 215.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 221. In an embodiment, the polynucleotide, e.g., second polynucleotide encoding the GM-CSF molecule comprises the nucleotide sequence of SEQ ID NO: 221. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 219. In an embodiment, the polynucleotide, e.g., second polynucleotide encoding the GM-CSF molecule comprises the nucleotide sequence of SEQ ID NO: 219. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment, the polynucleotide, e.g., second polynucleotide encoding the GM-CSF molecule comprises a nucleotide sequence having at least 85%, 90%, 95%, 96%, 97%, 98%, 99% or 100% identity to the nucleic acid sequence of SEQ ID NO: 224. In an embodiment, the polynucleotide, e.g., second polynucleotide encoding the GM-CSF molecule comprises the nucleotide sequence of SEQ ID NO: 224. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 220.

In an embodiment of any of the methods or compositions for use disclosed herein, the GM-CSF molecule comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the albumin is HSA comprising an amino acid sequence having at least 85%, 90%, 95%, 96%, 97%, 98%, 99% or 100% identity to the amino acid sequence of SEQ ID NO:8. In an embodiment, the albumin is HSA comprising the amino acid sequence of SEQ ID NO:8.

In an embodiment of any of the methods or compositions for use disclosed herein, the GM-CSF molecule further comprises a targeting moiety, e.g., a dendritic cell targeting moiety, or a tissue-specific targeting moiety. In an embodiment, the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

In an aspect, the disclosure provides a kit comprising a container comprising a lipid nanoparticle (LNP) composition disclosed herein, or a pharmaceutical composition disclosed herein.

In an embodiment, the kit comprises a package insert comprising instructions for administration of the lipid nanoparticle or pharmaceutical composition for treating or delaying a disease associated with aberrant T regulatory cell function in an individual.

In an embodiment, the lipid nanoparticle composition comprises a pharmaceutically acceptable carrier.

Additional features of any of the LNP compositions, pharmaceutical composition comprising said LNPs, methods or compositions for use disclosed herein include the following embodiments.

In an embodiment of any of the LNP compositions, methods or compositions for use disclosed herein, the first and second polynucleotides are formulated at an (a):(b) mass ratio of 10:1, 8:1, 6:1, 4:1, 3:1, 2:1, 1.5:1, or 1:1. In an embodiment, the first and second polynucleotides are formulated at an (a):(b) mass ratio of 1:1.5, 1:2, 1:3, 1:4, 1:6, 1:8, or 1:10. In an embodiment, the first and second polynucleotides are formulated at an (a):(b) mass ratio of 1:1.

In an embodiment of any of the LNP compositions, methods or compositions for use disclosed herein, the LNP composition increases the level and/or activity of T regulatory cells and/or suppressor T cells, e.g., as determined by an assay in a sample (e.g., a sample from a subject). In an embodiment, the T regulatory cells comprise FoxP3+ expressing and/or CD25+ expressing T regulatory cells. In an embodiment, the T regulatory cells are CD4+ and/or CD8+ T regulatory cells. In an embodiment, the increase in level and/or activity of T regulatory cells occurs in vitro or in vivo.

In an embodiment, the increase in level and/or activity of T regulatory cells is compared to level and/or activity of T regulatory cells in an otherwise similar sample which is: not contacted with the LNP composition comprising (a) and (b); or contacted with a composition comprising only (a) or a composition comprising only (b).

In an embodiment, the increase in level and/or activity of T regulatory cells comprises a one, or all or a combination (e.g., 2, 3, or all) of the following parameters:
(a) increased level of (e.g., number or proportion of) T regulatory cells (e.g., FoxP3+ T regulatory cells);
(b) increased activity of STAT5, e.g., STAT5 phosphorylation, in T regulatory cells (e.g., FoxP3+ T regulatory cells);
(c) increased activity or expression level of CTLA-4, TIGIT, ICOS and/or GITR in T regulatory cells (e.g., FoxP3+ T regulatory cells); and
(d) decreased activity or expression level of TGF beta and/or IL-10.

In an embodiment, the LNP composition increases the level of (e.g., number or proportion of) FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1-3 or 8.

In an embodiment, increase in the level of Fox P3+ T regulatory cells is compared to an otherwise similar population of cells not contacted with a composition comprising IL-2 and GM-CSF.

In an embodiment, the LNP composition increases in the activity of STAT5 (e.g., STAT5 phosphorylation) in FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1. In an embodiment, the increase in activity of STAT5 is compared to the activity of STAT5 in FoxP3− cells or Natural Killer cells

In an embodiment, the LNP composition increases in the activity and/or expression level of one or more (e.g., two, three, or all) of CTLA-4, TIGIT, ICOS and/or GITR in T regulatory cells (e.g., FoxP3+ T regulatory cells), e.g., a 1.5 to 10 fold (e.g., 2 to 8 fold, 3 to 7 fold, 4 to 6 fold, 1.5 to 10 fold, 1.5 to 8 fold, 1.5 to 6 fold, 1.5 to 4 fold, 8 to 10 fold, 6 to 10 fold, or 4 to 10 fold) increase, as measured by an assay in Example 2. In an embodiment, the increase in activity and/or expression level of one or more (e.g., two, three, or all) of CTLA-4, TIGIT, ICOS and/or GITR in T regulatory cells is compared to an otherwise similar population of cells not contacted with a composition comprising IL-2 and GM-CSF.

In an embodiment, the composition increases T regulatory cells (e.g., CD25+ T regulatory cells) as compared to type 1 T helper cells (Tₕ1) cells; type 2 T helper cells (Tₕ2) cells; and/or type 17 T helper cells (Tₕ17) cells.

In an embodiment, the increase in level and/or activity of suppressor T cells comprises one or both of the following parameters: (a) increased activity or expression level of Lag 3; and/or (b) increased activity or expression level of CD94b. In an embodiment, the increase in level and/or activity of suppressor T cells is compared to level and/or activity of suppressor T cells in an otherwise similar sample which is: not contacted with the composition comprising (a) and (b); or contacted with a composition comprising only (a) or a composition comprising only (b). In an embodiment, the increase in level and/or activity of suppressor T cells occurs in vitro or in vivo.

In an embodiment of any of the LNP compositions, methods or compositions for use disclosed herein, the first polynucleotide, the second polynucleotide, or both, comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, each mRNA in the lipid nanoparticle comprises fully modified N1-methylpseudouridine.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid.

In an embodiment of any of the LNP compositions, methods or compositions for use disclosed herein, the LNP composition comprises an ionizable lipid comprising an amino lipid. In an embodiment, the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8). In an embodiment, the ionizable lipid comprises a compound of Formula (I I). In an embodiment, the ionizable lipid comprises Compound 18. In an embodiment, the ionizable lipid comprises Compound 25.

In an embodiment of any of the LNP compositions, methods or compositions for use disclosed herein, the LNP composition comprises a non-cationic helper lipid or phospholipid comprising a compound selected from the group consisting of DSPC, DPPC, DMPC, DMPE, DOPC, Compound H-409, Compound H-418, Compound H-420, Compound H-421 and Compound H-422. In an embodiment, the phospholipid is DSPC. In an embodiment, the phospholipid is DMPE. In an embodiment, the phospholipid is Compound H-409.

In an embodiment of any of the LNP compositions, methods or compositions for use disclosed herein, the LNP composition comprises a structural lipid. In one embodiment, the structural lipid is a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In one embodiment, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In one embodiment, the phytosterol is a combination of Compound S-141, Compound S-140, Compound S-143 and Compound S-148. In one embodiment, the phytosterol comprises a sitosterol or a salt or an ester thereof. In one embodiment, the phytosterol comprises a stigmasterol or a salt or an ester thereof. In one embodiment, the phytosterol is beta-sitosterol

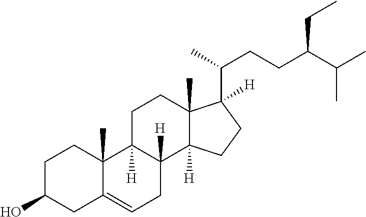

or a salt or an ester thereof.

In one embodiment of the LNPs or methods of the disclosures, the LNP comprises a phytosterol, or a salt or ester thereof, and cholesterol or a salt thereof.

In some embodiments, the phytosterol or a salt or ester thereof is selected from the group consisting of β-sitosterol, β-sitostanol, campesterol, and brassicasterol, and combinations thereof.

In one embodiment, the phytosterol is β-sitosterol. In one embodiment, the phytosterol is β-sitostanol. In one embodiment, the phytosterol is campesterol. In one embodiment, the phytosterol is brassicasterol.

In some embodiments, the phytosterol or a salt or ester thereof is selected from the group consisting of β-sitosterol, and stigmasterol, and combinations thereof. In one embodiment, the phytosterol is β-sitosterol. In one embodiment, the phytosterol is stigmasterol.

In some embodiments of the LNPs or methods of the disclosure, the LNP comprises a sterol, or a salt or ester thereof, and cholesterol or a salt thereof, and the sterol or a salt or ester thereof is selected from the group consisting of β-sitosterol-d7, brassicasterol, Compound S-30, Compound S-31 and Compound S-32.

In one embodiment, the structural lipid is selected from selected from β-sitosterol and cholesterol. In an embodiment, the structural lipid is β-sitosterol. In an embodiment, the structural lipid is cholesterol.

In an embodiment of any of the LNP compositions, methods or compositions for use disclosed herein, the LNP composition comprises a PEG lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. In one embodiment, the PEG lipid is selected from the group consisting of Compound 428, Compound P-L16, Compound P-L17, Compound P-L18, Compound P-L19, Compound P-L1, and Compound P-L2. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In one embodiment, the PEG lipid is selected from the group consisting of Compound P-L3, Compound P-L4, Compound P-L6, Compound P-L8, Compound P-L9 and Compound P-L25. In an embodiment, the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In an embodiment, the PEG lipid comprises a compound selected from the group consisting of Compound P-428, Compound PL-16, Compound PL-17, Compound PL-18, Compound PL-19, Compound PL-1, and Compound PL-2. In an embodiment, the PEG lipid comprises Compound P-428.

In an embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In an embodiment, the PEG lipid is selected from the group consisting of PEG-c-DOMG, PEG-DMG, PEG-DLPE, PEG-DMPE, PEG-DPPC and PEG-DSPE lipid. In an embodiment, the PEG-lipid is PEG-DMG.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In an embodiment, the ionizable lipid of (i) comprises Compound 18; the sterol lipid of (ii) comprises cholesterol; the non-cationic helper lipid or phospholipid of (iii) comprises DSPC and the PEG-lipid of (iv) comprises compound P-428.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In an embodiment, the ionizable lipid of (i) comprises Compound 25; the sterol lipid of (ii) comprises cholesterol; the non-cationic helper lipid or phospholipid of (iii) comprises DSPC and the PEG-lipid of (iv) comprises compound P-428.

In an embodiment of any of the LNP compositions, methods or compositions for use disclosed herein, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid.

In one embodiment of the LNPs or methods of the disclosure, the LNP comprises about 49.83 mol % ionizable lipid, about 9.83 mol % non-cationic helper lipid or phospholipid, about 30.33 mol % sterol or other structural lipid, and about 2.0 mol % PEG lipid.

In one embodiment of the LNPs or methods of the disclosure, the LNP comprises about 47.5 mol % ionizable lipid, about 10.5 mol % non-cationic helper lipid or phospholipid, about 39 mol % sterol or other structural lipid, and about 3.0 mol % PEG lipid.

In an embodiment of any of the LNP compositions, methods or compositions for use disclosed herein, the LNP comprises about 45 mol % to about 50 mol % ionizable lipid. In one embodiment of the LNPs or methods of the disclosure, the LNP comprises about 45.5 mol % to about 49.5 mol % ionizable lipid. In one embodiment of the LNPs or methods of the disclosure, the LNP comprises about 46 mol % to about 49 mol % ionizable lipid. In one embodiment of the LNPs or methods of the disclosure, the LNP comprises about 46.5 mol % to about 48.5 mol % ionizable lipid. In one embodiment of the LNPs or methods of the disclosure, the LNP comprises about 47 mol % to about 48 mol % ionizable lipid.

In an embodiment of any of the LNP compositions, methods or compositions for use disclosed herein, the LNP comprises about 45 mol % to about 49.5 mol % ionizable lipid. In one embodiment of the LNPs or methods of the disclosure, the LNP comprises about 45 mol % to about 49 mol % ionizable lipid. In one embodiment of the LNPs or methods of the disclosure, the LNP comprises about 45 mol % to about 48.5 mol % ionizable lipid. In one embodiment of the LNPs or methods of the disclosure, the LNP comprises about 45 mol % to about 48 mol % ionizable lipid. In one embodiment of the LNPs or methods of the disclosure, the LNP comprises about 45 mol % to about 47.5 mol % ionizable lipid. In one embodiment of the LNPs or methods of the disclosure, the LNP comprises about 45 mol % to about 47 mol % ionizable lipid. In one embodiment of the LNPs or methods of the disclosure, the LNP comprises about 45 mol % to about 46.5 mol % ionizable lipid. In one embodiment of the LNPs or methods of the disclosure, the LNP comprises about 45 mol % to about 46 mol % ionizable lipid. In one embodiment of the LNPs or methods of the disclosure, the LNP comprises about 45 mol % to about 45.5 mol % ionizable lipid.

In an embodiment of any of the LNP compositions, methods or compositions for use disclosed herein, the LNP comprises about 45.5 mol % to about 50 mol % ionizable lipid. In one embodiment of the LNPs or methods of the disclosure, the LNP comprises about 46 mol % to about 50 mol % ionizable lipid. In one embodiment of the LNPs or methods of the disclosure, the LNP comprises about 46.5 mol % to about 50 mol % ionizable lipid. In one embodiment of the LNPs or methods of the disclosure, the LNP comprises about 47 mol % to about 50 mol % ionizable lipid. In one embodiment of the LNPs or methods of the disclosure, the LNP comprises about 47.5 mol % to about 50 mol % ionizable lipid. In one embodiment of the LNPs or methods of the disclosure, the LNP comprises about 48 mol % to about 50 mol % ionizable lipid. In one embodiment of the LNPs or methods of the disclosure, the LNP comprises about 48.5 mol % to about 50 mol % ionizable lipid. In one embodiment of the LNPs or methods of the disclosure, the LNP comprises about 49 mol % to about 50 mol % ionizable lipid. In one embodiment of the LNPs or methods of the disclosure, the LNP comprises about 49.5 mol % to about 50 mol % ionizable lipid.

In an embodiment of any of the LNP compositions, methods or compositions for use disclosed herein, the LNP comprises about 45 mol % to about 46 mol % ionizable lipid. In one embodiment of the LNPs or methods of the disclosure, the LNP comprises about 45.5 mol % to about 46.5 mol % ionizable lipid. In one embodiment of the LNPs or methods of the disclosure, the LNP comprises about 46 mol % to about 47 mol % ionizable lipid. In one embodiment of the LNPs or methods of the disclosure, the LNP comprises about 46.5 mol % to about 47.5 mol % ionizable lipid. In one embodiment of the LNPs or methods of the disclosure, the LNP comprises about 47 mol % to about 48 mol % ionizable lipid. In one embodiment of the LNPs or methods of the disclosure, the LNP comprises about 47.5 mol % to about 48.5 mol % ionizable lipid. In one embodiment of the LNPs or methods of the disclosure, the LNP comprises about 48 mol % to about 49 mol % ionizable lipid. In one embodiment of the LNPs or methods of the disclosure, the LNP comprises about 48.5 mol % to about 49.5 mol % ionizable lipid. In one embodiment of the LNPs or methods of the disclosure, the LNP comprises about 49 mol % to about 50 mol % ionizable lipid.

In an embodiment of any of the LNP compositions, methods or compositions for use disclosed herein, the LNP comprises about 45 mol % ionizable lipid. In one embodiment of the LNPs or methods of the disclosure, the LNP comprises about 45.5 mol % ionizable lipid. In one embodiment of the LNPs or methods of the disclosure, the LNP comprises about 46 mol % ionizable lipid. In one embodiment of the LNPs or methods of the disclosure, the LNP comprises about 46.5 mol % ionizable lipid. In one embodiment of the LNPs or methods of the disclosure, the LNP comprises about 47 mol % ionizable lipid. In one embodiment of the LNPs or methods of the disclosure, the LNP comprises about 47.5 mol % ionizable lipid. In one embodiment of the LNPs or methods of the disclosure, the LNP comprises about 48 mol % ionizable lipid. In one embodiment of the LNPs or methods of the disclosure, the LNP comprises about 48.5 mol % ionizable lipid. In one embodiment of the LNPs or methods of the disclosure, the LNP comprises about 49 mol % ionizable lipid. In one embodiment of the LNPs or methods of the disclosure, the LNP comprises about 49.5 mol % ionizable lipid. In one embodiment of the LNPs or methods of the disclosure, the LNP comprises about 50 mol % ionizable lipid.

In an embodiment of any of the LNP compositions, methods or compositions for use disclosed herein, the LNP comprises about 1 mol % to about 5 mol % PEG lipid. In one embodiment of the LNPs or methods of the disclosure, the LNP comprises about 1.5 mol % to about 4.5 mol % PEG lipid. In one embodiment of the LNPs or methods of the disclosure, the LNP comprises about 2 mol % to about 4 mol % PEG lipid. In one embodiment of the LNPs or methods of the disclosure, the LNP comprises about 2.5 mol % to about 3.5 mol % PEG lipid.

In an embodiment of any of the LNP compositions, methods or compositions for use disclosed herein, the LNP comprises about 1 mol % to about 4.5 mol % PEG lipid. In one embodiment of the LNPs or methods of the disclosure, the LNP comprises about 1 mol % to about 4 mol % PEG lipid. In one embodiment of the LNPs or methods of the disclosure, the LNP comprises about 1 mol % to about 3.5 mol % PEG lipid. In one embodiment of the LNPs or methods of the disclosure, the LNP comprises about 1 mol % to about 3 mol % PEG lipid. In one embodiment of the LNPs or methods of the disclosure, the LNP comprises about 1 mol % to about 2.5 mol % PEG lipid. In one embodiment of the LNPs or methods of the disclosure, the LNP comprises about 1 mol % to about 2 mol % PEG lipid. In one embodiment of the LNPs or methods of the disclosure, the LNP comprises about 1 mol % to about 1.5 mol % PEG lipid.

In an embodiment of any of the LNP compositions, methods or compositions for use disclosed herein, the LNP comprises about 1.5 mol % to about 5 mol % PEG lipid. In one embodiment of the LNPs or methods of the disclosure, the LNP comprises about 2 mol % to about 5 mol % PEG lipid. In one embodiment of the LNPs or methods of the disclosure, the LNP comprises about 2.5 mol % to about 5 mol % PEG lipid. In one embodiment of the LNPs or methods of the disclosure, the LNP comprises about 3 mol % to about 5 mol % PEG lipid. In one embodiment of the LNPs or methods of the disclosure, the LNP comprises about 3.5 mol % to about 5 mol % PEG lipid. In one embodiment of the LNPs or methods of the disclosure, the LNP comprises about 4 mol % to about 5 mol % PEG lipid. In one embodiment of the LNPs or methods of the disclosure, the LNP comprises about 4.5 mol % to about 5 mol % PEG lipid.

In one embodiment of the LNPs or methods of the disclosure, the LNP comprises about 1 mol % to about 2 mol % PEG lipid. In one embodiment of the LNPs or methods of the disclosure, the LNP comprises about 1.5 mol % to about 2.5 mol % PEG lipid. In one embodiment of the LNPs or methods of the disclosure, the LNP comprises about 2 mol % to about 3 mol % PEG lipid. In one embodiment of the LNPs or methods of the disclosure, the LNP comprises about 3.5 mol % to about 4.5 mol % PEG lipid. In one embodiment of the LNPs or methods of the disclosure, the LNP comprises about 4 mol % to about 5 mol % PEG lipid.

In an embodiment of any of the LNP compositions, methods or compositions for use disclosed herein, the LNP comprises about 1 mol % PEG lipid. In one embodiment of the LNPs or methods of the disclosure, the LNP comprises about 1.5 mol % PEG lipid. In one embodiment of the LNPs or methods of the disclosure, the LNP comprises about 2 mol % PEG lipid. In one embodiment of the LNPs or methods of the disclosure, the LNP comprises about 2.5 mol % PEG lipid. In one embodiment of the LNPs or methods of the disclosure, the LNP comprises about 3 mol % PEG lipid. In one embodiment of the LNPs or methods of the disclosure, the LNP comprises about 3.5 mol % PEG lipid. In one embodiment of the LNPs or methods of the disclosure, the LNP comprises about 4 mol % PEG lipid. In one embodiment of the LNPs or methods of the disclosure, the LNP comprises about 4.5 mol % PEG lipid. In one embodiment of the LNPs or methods of the disclosure, the LNP comprises about 5 mol % PEG lipid.

In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In an embodiment of any of the LNP compositions, methods or compositions for use disclosed herein, the LNP comprises about 20 mol % to about 60 mol % Compound 18, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % Compound 18, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % Compound 18, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % Compound 18, about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 47.5 mol % of Compound 18, about 10.5 mol % non-cationic helper lipid or phospholipid, about 39 mol % sterol or other structural lipid, and about 3.0 mol % PEG lipid.

In an embodiment of any of the LNP compositions, methods or compositions for use disclosed herein, the LNP comprises about 20 mol % to about 60 mol % Compound 18, about 5 mol % to about 25 mol % DSPC as the non-cationic helper lipid or phospholipid, about 25 mol % to about 55 mol % cholesterol as the sterol lipid, and about 0.5 mol % to about 15 mol % Compound P-428 as the PEG lipid. In one embodiment of the LNPs or methods of the disclosure, the LNP comprises about 35 mol % to about 55 mol % Compound 18, about 5 mol % to about 25 mol % DSPC as the non-cationic helper lipid or phospholipid, about 30 mol % to about 40 mol % cholesterol as the sterol lipid, and about 0 mol % to about 10 mol % Compound P-428 as the PEG lipid. In one embodiment of the LNPs or methods of the disclosure, the LNP comprises about 50 mol % Compound 18, about 10 mol % DSPC as the non-cationic helper lipid or phospholipid, about 38.5 mol % cholesterol as the sterol lipid, and about 1.5 mol % Compound P-428 as the PEG lipid. In one embodiment of the LNPs or methods of the disclosure, the LNP comprises about 49.83 mol % Compound 18, about 9.83 mol % non-cationic DSPC as the helper lipid or phospholipid, about 30.33 mol % cholesterol as the sterol lipid, and about 2.0 mol % Compound P-428 as the PEG lipid. In one embodiment of the LNPs or methods of the disclosure, the LNP comprises about 47.5 mol % of Compound 18, about 10.5 mol % DSPC as the non-cationic helper lipid or phospholipid, about 39 mol % cholesterol as the sterol lipid, and about 3.0 mol % Compound P-428 as the PEG lipid.

In an embodiment of any of the LNP compositions, methods or compositions for use disclosed herein, the LNP comprises about 20 mol % to about 60 mol % Compound 25, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % Compound 25, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % Compound 25, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % Compound 25, about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 47.5 mol % of Compound 25, about 10.5 mol % non-cationic helper lipid or phospholipid, about 39 mol % sterol or other structural lipid, and about 3.0 mol % PEG lipid.

In an embodiment of any of the LNP compositions, methods or compositions for use disclosed herein, the LNP comprises about 20 mol % to about 60 mol % Compound 25, about 5 mol % to about 25 mol % DSPC as the non-cationic helper lipid or phospholipid, about 25 mol % to about 55 mol % cholesterol as the sterol lipid, and about 0.5 mol % to about 15 mol % Compound P-428 as the PEG lipid. In one embodiment of the LNPs or methods of the disclosure, the LNP comprises about 35 mol % to about 55 mol % Compound 25, about 5 mol % to about 25 mol % DSPC as the non-cationic helper lipid or phospholipid, about 30 mol % to about 40 mol % cholesterol as the sterol lipid, and about 0 mol % to about 10 mol % Compound P-428 as the PEG lipid. In one embodiment of the LNPs or methods of the disclosure, the LNP comprises about 50 mol % Compound 25, about 10 mol % DSPC as the non-cationic helper lipid or phospholipid, about 38.5 mol % cholesterol as the sterol lipid, and about 1.5 mol % Compound P-428 as the PEG lipid. In one embodiment of the LNPs or methods of the disclosure, the LNP comprises about 49.83 mol % Compound 25, about 9.83 mol % non-cationic DSPC as the helper lipid or phospholipid, about 30.33 mol % cholesterol as the sterol lipid, and about 2.0 mol % Compound P-428 as the PEG lipid. In one embodiment of the LNPs or methods of the disclosure, the LNP comprises about 47.5 mol % of Compound 25, about 10.5 mol % DSPC as the non-cationic helper lipid or phospholipid, about 39 mol % cholesterol as the sterol lipid, and about 3.0 mol % Compound P-428 as the PEG lipid.

In an embodiment of any of the LNP compositions, methods or compositions for use disclosed herein, the LNP is formulated for intravenous, subcutaneous, intramuscular, intraocular, intranasal, rectal or oral delivery. In an embodiment, the LNP is formulated for intravenous delivery. In an embodiment, the LNP is formulated for subcutaneous delivery. In an embodiment, the LNP is formulated for intramuscular delivery. In an embodiment, the LNP is formulated for intraocular delivery. In an embodiment, the LNP is formulated for intranasal delivery. In an embodiment, the LNP is formulated for rectal delivery. In an embodiment, the LNP is formulated for oral delivery.

In an embodiment of any of the LNP compositions, methods or compositions for use disclosed herein, wherein the LNP is administered at a dose disclosed herein. In an embodiment, the dose, e.g., effective dose, of the first polynucleotide encoding the IL-2 molecule in the lipid nanoparticle is at least 10%, 20%, 30%, 40%, 50%, 60%, 70%, 80% or 95% lesser than the dose of a naturally occurring, or recombinant IL-2, e.g., in an otherwise similar LNP.

In an embodiment of any of the methods or compositions for use disclosed herein, the first LNP and the second LNP are administered sequentially or simultaneously. In an embodiment, first LNP and the second LNP are administered sequentially. In an embodiment, first LNP is administered first and the second LNP is administered second. In an embodiment, first LNP is administered second and the second LNP is administered first. In an embodiment, first LNP and the second LNP are administered simultaneously.

In an embodiment, first LNP and the second LNP are administered in the same or in separate compositions. In an embodiment, the first LNP comprising the first polynucleotide encoding the IL-2 molecule is administered first and the second LNP comprising the second polynucleotide encoding the GM-CSF molecule is administered second. In an embodiment, the first polynucleotide encoding the IL-2 molecule is administered at least 1 day, 2 days, 3 days, 4 days, 5 days, 6 days, 7 days, 8 days, 9 days, or 10 days (e.g., 7 days), before administration of the second LNP comprising the second polynucleotide encoding the GM-CSF molecule. In an embodiment, the first LNP comprising the first polynucleotide encoding the IL-2 molecule is administered at least 1 week, 2 weeks, 3 weeks, 4 weeks, 5 weeks, or 6 weeks, before administration of the second LNP comprising the second polynucleotide encoding the GM-CSF molecule.

In an embodiment, the first LNP comprising the first polynucleotide encoding the IL-2 molecule is administered second and the second LNP comprising the second polynucleotide encoding the GM-CSF molecule is administered first. In an embodiment, the first LNP comprising the first polynucleotide encoding the IL-2 molecule is administered at least 1 day, 2 days, 3 days, 4 days, 5 days, 6 days, 7 days, 8 days, 9 days, or 10 days (e.g., 7 days), after administration of the second LNP comprising the second polynucleotide encoding the GM-CSF molecule. In an embodiment, the first LNP comprising the first polynucleotide encoding the IL-2 molecule is administered at least 1 week, 2 weeks, 3 weeks, 4 weeks, 5 weeks, or 6 weeks, after administration of the second LNP comprising the second polynucleotide encoding the GM-CSF molecule.

In an embodiment of any of the methods or compositions for use disclosed herein, the LNP (e.g., the first and/or second LNP) is administered according to a dosing interval, e.g., as described herein. In an embodiment, the dosing interval comprises:
(a) an initial dose of the first LNP and one or more subsequent doses of the second LNP;
(b) an initial dose of the second LNP and one or more subsequent doses of the first LNP;
(c) an initial dose of the first LNP followed by one or more subsequent doses of a combination of the first LNP and the second LNP;
(d) an initial dose of the second LNP followed by one or more subsequent doses of a combination of the first LNP and the second LNP; and/or
(e) one or more doses of the initial dose of the first LNP or the second LNP.

In an embodiment, the dosing interval comprises an initial dose of the first LNP and one or more subsequent doses of the second LNP. In an embodiment, the dosing interval comprises an initial dose of the second LNP and one or more subsequent doses of the first LNP. In an embodiment, the dosing interval comprises an initial dose of the first LNP followed by one or more subsequent doses of a combination of the first LNP and the second LNP. In an embodiment, the dosing interval comprises an initial dose of the second LNP followed by one or more subsequent doses of a combination of the first LNP and the second LNP. In an embodiment, the dosing interval comprises an initial dose of the second LNP followed by one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, 1-5 doses) of a combination of the first LNP and the second LNP.

In an embodiment, the dosing interval is performed over at least 1 week, 2 weeks, 3 weeks, or 4 weeks.

In an embodiment, the one or more subsequent doses of the combination of the first LNP and second LNP are administered, e.g., at least 5-20 days, 5-19 days, 5-18 days, 5-17 days, 5-16 days, 5-15 days, 5-14 days, 5-13 days, 5-12 days, 5-11 days, 5-10 days, 5-9 days, 5-8 days, 5-7 days, 5-6 days, 6-20 days, 7-20 days, 8-20 days, 9-20 days, 10-20 days, 11-20 days, 12-20 days, 13-20 days, 14-20 days, 15-20 days, 16-20 days, 17-20 days, 18-20 days, or 19-20 days, e.g., 7-14 days, after administration of the initial dose of the second LNP In an embodiment, the dosing interval is repeated at least 1 time, at least 2 times, at least 3 times, at least 4 times, at least 5 times, at least 6 times, at least 7 times, at least 8 times, at least 9 times, or at least 10 times.

In an embodiment, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years or 5 years.

In an embodiment of any of the methods or compositions for use disclosed herein, an initial dose of an LNP (e.g., an LNP described herein) is at least 10%, 20%, 25%, 30%, 35%, 40%, 45%, 50%, 55%, 60%, 65%, 70%, 75%, 80%, 85%, or 90% lower than a subsequent dose of an LNP (e.g., the same LNP). In an embodiment, the initial dose of the first LNP comprising IL-2 is at least 10%, 20%, 25%, 30%, 35%, 40%, 45%, 50%, 55%, 60%, 65%, 70%, 75%, 80%, 85%, or 90% lower than the subsequent dose of the first LNP comprising IL-2 (e.g., administered alone or in combination with the second LNP comprising GM-CSF). In an embodiment, the initial dose of the second LNP comprising the second polynucleotide encoding the GM-CSF is at least 10%, 20%, 25%, 30%, 35%, 40%, 45%, 50%, 55%, 60%, 65%, 70%, 75%, 80%, 85%, or 90% lower than the subsequent dose of the second LNP comprising the second polynucleotide encoding the GM-CSF (e.g., administered alone or in combination with the first LNP comprising IL-2).

In an embodiment of any of the methods or compositions for use disclosed herein, the disease associated with an aberrant T regulatory cell function is an autoimmune disease, or a disease with hyper-activated immune function. In an embodiment, the disease is an autoimmune disease. In an embodiment, the autoimmune disease is chosen from: rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)); multiple sclerosis; autoimmune hepatitis (e.g., Type 1 or Type 2); primary biliary cholangitis; organ transplant associated rejection; myasthenia gravis; Parkinsons's Disease; Alzheimer's Disease; amyotrophic lateral sclerosis; psoriasis; or polymyositis (also known as dermatomyositis).

In an embodiment, the autoimmune disease is rheumatoid arthritis (RA).

In an embodiment, the autoimmune disease is graft versus host disease (GVHD) (e.g., acute GVHD or chronic GVHD).

In an embodiment, the autoimmune disease is diabetes, e.g., Type 1 diabetes.

In an embodiment, the autoimmune disease is inflammatory bowel disease (IBD), e.g., colitis, ulcerative colitis or Crohn's disease.

In an embodiment, the autoimmune disease is lupus, e.g., systemic lupus erythematosus (SLE).

In an embodiment, the autoimmune disease is multiple sclerosis.

In an embodiment, the autoimmune disease is autoimmune hepatitis, e.g., Type 1 or Type 2.

In an embodiment, the autoimmune disease is primary biliary cholangitis. In an embodiment, an organ transplant associated rejection comprises renal allograft rejection; liver transplant rejection; bone marrow transplant rejection; or stem cell transplant rejection. In an embodiment, a stem cell transplant comprises a transplant of any one or all of the following types of cells: stem cells, cord blood stem cells, hematopoietic stem cells, embryonic stem cells, cells derived from or comprising mesenchymal stem cells, and/or induced stem cells (e.g., induced pluripotent stem cells). In an embodiment, the stem cell comprises a pluripotent stem cell.

In an embodiment, the autoimmune disease is myasthenia gravis.

In an embodiment, the autoimmune disease is Parkinson's disease.

In an embodiment, the autoimmune disease is Alzheimer's disease.

In an embodiment, the autoimmune disease is amyotrophic lateral sclerosis.

In an embodiment, the autoimmune disease is psoriasis.

In an embodiment, the autoimmune disease is polymyositis.

In an embodiment of any of the methods or compositions for use disclosed herein, the subject is a mammal, e.g., a human.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

E1. A lipid nanoparticle (LNP) composition comprising a polynucleotide comprising an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables 1A, 2A or 4A.
E2. The LNP composition of embodiment 1, wherein the IL-2 molecule comprises a naturally occurring IL-2 molecule, a fragment of a naturally occurring IL-2 molecule, or a variant thereof.
E3. The LNP composition of embodiment 1 or 2, wherein the IL-2 molecule comprises a variant of a naturally occurring IL-2 molecule (e.g., an IL-2 variant, e.g., as described herein), or a fragment thereof.
E4. The LNP composition of any one of embodiments 1 to 3, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).
E5. The LNP composition of any one of embodiments 1 to 4, wherein the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.
E6. The LNP composition of any one of embodiments 2 to 5, wherein the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.
E7. The LNP composition of any one of embodiments 2 to 6, wherein the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.
E8. The LNP composition of any one of embodiments 2 to 7, wherein the IL-2 variant comprises a mutation, e.g., substitution, at position 88 of the IL-2 polypeptide sequence, e.g., an N88D substitution.
E9. The LNP composition of any one of embodiments 2 to 8, wherein the IL-2 variant comprises a mutation, e.g., substitution, at position 91 of the IL-2 polypeptide sequence, e.g., a V91K substitution.
E10. The LNP composition of any one of embodiments 2 to 9, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 88 of the IL-2 polypeptide sequence, e.g., an N88D substitution.
   E11. The LNP composition of any one of embodiments 2 to 9, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 91 of the IL-2 polypeptide sequence, e.g., a V91K substitution.
   E12. The LNP composition of any one of embodiments 2 to 11, wherein the IL-2 variant comprises a mutation, e.g., substitution, at position 125 of the IL-2 polypeptide sequence, e.g., a C125S substitution.
   E13. The LNP composition of any one of embodiments 1 to 12, wherein the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E14. The LNP composition of any one of embodiments 1 to 13, wherein the IL-2 molecule comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E15. The LNP composition of any one of embodiments 1 to 14, wherein the polynucleotide encoding the IL-2 molecule comprises a nucleotide sequence having at least 85%, 90%, 95%, 96%, 97%, 98%, 99% or 100% identity to the sequence of SEQ ID NO: 7, SEQ ID NO: 25, or SEQ ID NO: 36, optionally wherein the polynucleotide encoding the IL-2 molecule comprises a nucleotide sequence having at least 85%, 90%, 95%, 96%, 97%, 98%, 99% or 100% identity to the sequence of SEQ ID NO: 36, optionally wherein the polynucleotide encoding the IL-2 molecule comprises the nucleotide sequence of SEQ ID NO: 36.
   E16. The LNP composition of any one of embodiments 1 to 15, wherein the IL-2 molecule comprises a half-life extender, e.g., a protein (or fragment thereof) that binds to a serum protein such as albumin, IgG, FcRn or transferrin.
   E17. The LNP composition of any one of embodiments 1 to 16, wherein the half-life extender comprises albumin or a fragment thereof, or an Fc domain of an antibody molecule (e.g., an Fc domain with enhanced FcRn binding).
   E18. The LNP composition of any one of embodiments 1 to 17, wherein the half-life extender is albumin, or a fragment thereof.
   E19. The LNP composition of any one of embodiments 1 to 18, wherein the half-life extender is albumin, e.g., human serum albumin (HSA), mouse serum albumin (MSA), cyno serum albumin (CSA) or rat serum albumin (RSA).
   E20. The LNP composition of embodiment 19, wherein the albumin is HSA comprising an amino acid sequence having at least 85%, 90%, 95%, 96%, 97%, 98%, 99% or 100% identity to the amino acid sequence of SEQ ID NO: 8.
   E21. The LNP composition of any one of embodiments 1 to 20, wherein the IL-2 molecule comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without the leader sequence.
   E22. The LNP composition of any one of embodiments 1 to 21, wherein the IL-2 molecule comprises the amino acid sequence of SEQ ID NO: 9, SEQ ID NO: 10, SEQ ID NO: 11, SEQ ID NO: 12 or SEQ ID NO: 13 with or without the leader sequence.
   E23. The LNP composition of embodiment 22, wherein the IL-2 molecule comprises the amino acid sequence of SEQ ID NO:11.
   E24. The LNP composition of any one of embodiments 1 to 23, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 25;
(b) the nucleotide sequence of SEQ ID NO: 25; or
(c) the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.
   E25. The LNP composition of any one of embodiments 1 to 23, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 36;
(b) the nucleotide sequence of SEQ ID NO: 36; or
(c) the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.
   E26. The LNP composition of any one of embodiments 1 to 25, wherein the IL-2 molecule further comprises a targeting moiety, e.g., a T regulatory cell targeting moiety or a tissue-specific targeting moiety.
   E27. The LNP composition of embodiment 26, wherein the tissue-specific targeting moiety binds to ROS-CII, EDA, EDB, TnC A1, SyETP, GLUT-2, GD2, FAP, VCAM or MADCAM.
   E28. The LNP composition of embodiment 26, wherein the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E29. The LNP composition of embodiment 28, wherein the T regulatory cell targeting moiety binds to a molecule present on a T regulatory cell.
   E30. The LNP composition of embodiment 28 or 29, wherein the T regulatory cell targeting moiety comprises an antibody molecule that binds to CTLA-4, GITR, TLR8, or Nrp1.
   E31. The LNP composition of embodiment 30, wherein the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17.
   E32. The LNP composition of any one of embodiments 25-31, wherein the IL-2 molecule comprising the targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20.
   E33. The LNP composition of any one of embodiments 25-32, wherein the IL-2 molecule comprising the targeting moiety comprises the amino acid sequence of SEQ ID NO: 18, SEQ ID NO: 19, or SEQ ID NO: 20.
   E34. The LNP composition of any one of embodiments 25-31, wherein the IL-2 molecule comprising the targeting moiety is encoded by a nucleotide sequence having at least 85%, 90%, 95%, 96%, 97%, 98%, 99% or 100% identity to the nucleic acid sequence of SEQ ID NO: 21, SEQ ID NO:22 or SEQ ID NO: 23.
   E35. A lipid nanoparticle (LNP) composition comprising a polynucleotide comprising an mRNA which encodes a GM-CSF molecule comprising an amino acid sequence having at least 85%, 90%, 95%, 96%, 97%, 98%, 99% or 100% identity to the amino acid sequence of a GM-CSF molecule provided in Table 3A or 3B.
   E36. The LNP composition of embodiment 35, wherein the GM-CSF molecule comprises a naturally occurring GM-CSF molecule, a fragment of a naturally occurring GM-CSF molecule, or a variant thereof.
   E37. The LNP composition of embodiment 35 or 36, wherein the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E38. The LNP composition of any one of embodiments 35-37, wherein the GM-CSF molecule comprises the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E39. The LNP composition of any one of embodiments 35-38, wherein the polynucleotide encoding the GM-CSF molecule comprises a nucleotide sequence having at least 85%, 90%, 95%, 96%, 97%, 98%, 99% or 100% identity to the nucleic acid sequence of SEQ ID NO: 15, SEQ ID NO: 38, SEQ ID NO: 40, SEQ ID NO: 42 or SEQ ID NO: 44, SEQ ID NO: 24, SEQ ID NO: 201, SEQ ID NO: 206, SEQ ID NO: 211, SEQ ID NO: 216, SEQ ID NO: 221, SEQ ID NO: 204, SEQ ID NO: 209, SEQ ID NO: 214, SEQ ID NO: 219, or SEQ ID NO: 224,
optionally wherein polynucleotide encoding the GM-CSF molecule comprises:
(a) a nucleotide sequence having at least 85%, 90%, 95%, 96%, 97%, 98%, 99% or 100% identity to the sequence of SEQ ID NO: 221;
(b) the nucleotide sequence of SEQ ID NO: 221; or
(c) the nucleotide sequence of SEQ ID NO: 224 which consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 29.
   E40. The LNP composition of any one of embodiments 35-39, wherein the GM-CSF molecule comprises a half-life extender, e.g., a protein (or fragment thereof) that binds to a serum protein such as albumin, IgG, FcRn or transferrin.
   E41. The LNP composition of embodiment 40, wherein the half-life extender comprises albumin or a fragment thereof, or an Fc domain of an antibody molecule (e.g., an Fc domain with enhanced FcRn binding).
   E42. The LNP composition of embodiment 40 or 41, wherein the half-life extender is albumin, or a fragment thereof.
   E43. The LNP composition of any one of embodiments 40-42, wherein the half-life extender is albumin, e.g., human serum albumin (HSA), mouse serum albumin (MSA), cyno serum albumin (CSA) or rat serum albumin (RSA).
   E44. The LNP composition of embodiment 43, wherein the albumin is HSA comprising an amino acid sequence having at least 85%, 90%, 95%, 96%, 97%, 98%, 99% or 100% identity to the amino acid sequence of SEQ ID NO:8.
   E45. The LNP composition of embodiment 43 or 44, wherein the GM-CSF molecule comprising HSA comprises an amino acid sequence having at least 85%, 90%, 95%, 96%, 97%, 98%, 99% or 100% identity to the amino acid sequence of SEQ ID NO: 16.
   E46. The LNP composition of any one of embodiments 43-45, wherein the GM-CSF molecule comprising HSA is encoded by a nucleotide sequence having at least 85%, 90%, 95%, 96%, 97%, 98%, 99% or 100% identity to the nucleic acid sequence of SEQ ID NO: 24, SEQ ID NO: 221, or SEQ ID NO: 224.
   E47. The LNP composition of any one of embodiments 35-46, wherein the GM-CSF molecule further comprises a targeting moiety, e.g., a dendritic cell targeting moiety, or a tissue-specific targeting moiety.
   E48. The LNP composition of embodiment 47, wherein the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E49. A lipid nanoparticle (LNP) composition, comprising:
(a) a first polynucleotide encoding an IL-2 molecule; and
(b) a second polynucleotide encoding a GM-CSF molecule,
wherein (a) and (b) comprise an mRNA, and optionally wherein, the first and second polynucleotides are formulated at an (a):(b) mass ratio of 1:1.
   E50. A lipid nanoparticle (LNP) composition, for stimulating T regulatory cells, the LNP composition comprising:
(a) a first polynucleotide encoding an IL-2 molecule; and
(b) a second polynucleotide encoding a GM-CSF molecule,
   wherein (a) and (b) comprise an mRNA.
   E51. The LNP composition of embodiment 49 or 50, wherein the IL-2 molecule comprises a naturally occurring IL-2 molecule, a fragment of a naturally occurring IL-2 molecule, or a variant thereof.
   E52. The LNP composition of any one of embodiments 49-51, wherein the IL-2 molecule comprises a variant of a naturally occurring IL-2 molecule (e.g., an IL-2 variant, e.g., as described herein), or a fragment thereof.
   E53. The LNP composition of any one of embodiments 49-52, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).
   E54. The LNP composition of any one of embodiments 49-53, wherein the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.
   E55. The LNP composition of any one of embodiments 49-54, wherein the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.
   E56. The LNP composition of any one of embodiments 52-55, wherein the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.
   E57. The LNP composition of any one of embodiments 52-56, wherein the IL-2 variant comprises a mutation, e.g., substitution, at position 88 of the IL-2 polypeptide sequence, e.g., an N88D substitution.
   E58. The LNP composition of any one of embodiments 52-57, wherein the IL-2 variant comprises a mutation, e.g., substitution, at position 91 of the IL-2 polypeptide sequence, e.g., a V91K substitution.
   E59. The LNP composition of any one of embodiments 52-56, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 88 of the IL-2 polypeptide sequence, e.g., an N88D substitution.
   E60. The LNP composition of any one of embodiments 52-56, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 91 of the IL-2 polypeptide sequence, e.g., a V91K substitution.
   E61. The LNP composition of any one of embodiments 52-60, wherein the IL-2 variant comprises a mutation, e.g., substitution, at position 125 of the IL-2 polypeptide sequence, e.g., a C125S substitution.
   E62. The LNP composition of any one of embodiments 49-61, wherein the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E63. The LNP composition of any one of embodiments 49-62, wherein the IL-2 molecule comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E64. The LNP composition of any one of embodiments 49-63, wherein the first polynucleotide comprises a nucleotide sequence having at least 85%, 90%, 95%, 96%, 97%, 98%, 99% or 100% identity to the sequence of SEQ ID NO: 7, SEQ ID NO: 25, or SEQ ID NO: 36, optionally wherein the polynucleotide encoding the IL-2 molecule comprises a nucleotide sequence having at least 85%, 90%, 95%, 96%, 97%, 98%, 99% or 100% identity to the sequence of SEQ ID NO: 36, optionally wherein the polynucleotide encoding the IL-2 molecule comprises the nucleotide sequence of SEQ ID NO: 36.
   E65. The LNP composition of any one of embodiments 49-64, wherein the IL-2 molecule comprises a half-life extender, e.g., a protein (or fragment thereof) that binds to a serum protein such as albumin, IgG, FcRn or transferrin.
   E66. The LNP composition of any one of embodiments 49-65, wherein the half-life extender comprises albumin or a fragment thereof, or an Fc domain of an antibody molecule (e.g., an Fc domain with enhanced FcRn binding).
   E67. The LNP composition of any one of embodiments 49-66, wherein the half-life extender is albumin, or a fragment thereof.
   E68. The LNP composition of any one of embodiments 49-67, wherein the half-life extender is albumin, e.g., human serum albumin (HSA), mouse serum albumin (MSA), cyno serum albumin (CSA) or rat serum albumin (RSA).
   E69. The LNP composition of embodiment 68, wherein the albumin is HSA comprising an amino acid sequence having at least 85%, 90%, 95%, 96%, 97%, 98%, 99% or 100% identity to the amino acid sequence of SEQ ID NO: 8.
   E70. The LNP composition of any one of embodiments 49-69, wherein the IL-2 molecule comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without the leader sequence.
   E71. The LNP composition of any one of embodiments 49-69, wherein the IL-2 molecule comprises the amino acid sequence of SEQ ID NO: 9, SEQ ID NO: 10, SEQ ID NO: 11, SEQ ID NO: 12 or SEQ ID NO: 13 with or without the leader sequence.
   E72. The LNP composition of embodiment 71, wherein the IL-2 molecule comprises the amino acid sequence of SEQ ID NO: 9.
   E73. The LNP composition of embodiment 71, wherein the IL-2 molecule comprises the amino acid sequence of SEQ ID NO: 10.
   E74. The LNP composition of embodiment 71, wherein the IL-2 molecule comprises the amino acid sequence of SEQ ID NO:11.
   E75. The LNP composition of embodiment 71, wherein the IL-2 molecule comprises the amino acid sequence of SEQ ID NO: 12.
   E76. The LNP composition of embodiment 71, wherein the IL-2 molecule comprises the amino acid sequence of SEQ ID NO: 13.
   E77. The LNP composition of any one of embodiments 49-71 or 74, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 25;
(b) the nucleotide sequence of SEQ ID NO: 25; or
(c) the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.
   E78. The LNP composition of any one of embodiments 49-71 or 74, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 36;
(b) the nucleotide sequence of SEQ ID NO: 36; or
(c) the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.
   E79. The LNP composition of any one of embodiments 49-78, wherein the IL-2 molecule further comprises a targeting moiety, e.g., a T regulatory cell targeting moiety or a tissue-specific targeting moiety.
   E80. The LNP composition of embodiment 79, wherein the tissue-specific targeting moiety binds to ROS-CII, EDA, EDB, TnC A1, SyETP, GLUT-2, GD2, FAP, VCAM or MADCAM.
   E81. The LNP composition of any one of embodiments 78-80, wherein the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E82. The LNP composition of embodiment 81, wherein the T regulatory cell targeting moiety binds to a molecule present on a T regulatory cell.
   E83. The LNP composition of embodiment 81 or 82, wherein the T regulatory cell targeting moiety comprises an antibody molecule that binds to CTLA-4, GITR, TLR8, or Nrp1.
   E84. The LNP composition of any one of embodiments 81-83, wherein the T regulatory cell targeting moiety comprises an antibody molecule that binds to CTLA-4.
   E85. The LNP composition of embodiment 84, wherein the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17.
   E86. The LNP composition of any one of embodiments 81-85, wherein the IL-2 molecule comprising the targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20.
   E87. The LNP composition of any one of embodiments 81-86, wherein the IL-2 molecule comprising the targeting moiety comprises the amino acid sequence of SEQ ID NO: 18, SEQ ID NO: 19, or SEQ ID NO: 20.
   E88. The LNP composition of any one of embodiments 81-86, wherein the IL-2 molecule comprising the targeting moiety is encoded by a nucleotide sequence having at least 85%, 90%, 95%, 96%, 97%, 98%, 99% or 100% identity to the nucleic acid sequence of SEQ ID NO: 21, SEQ ID NO:22 or SEQ ID NO: 23.
   E89. The LNP composition of any one of embodiments 49-88, wherein the GM-CSF molecule comprises a naturally occurring GM-CSF molecule, a fragment of a naturally occurring GM-CSF molecule, or a variant thereof.
   E90. The LNP composition of embodiment 89, wherein the GM-CSF molecule comprises an amino acid sequence having at least 85%, 90%, 95%, 96%, 97%, 98%, 99% or 100% identity to the amino acid sequence of SEQ ID NO: 14 SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E91. The LNP composition of embodiment 89, wherein the GM-CSF molecule comprises the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E92. The LNP composition of embodiment 89, wherein the second polynucleotide comprises a nucleotide sequence having at least 85%, 90%, 95%, 96%, 97%, 98%, 99% or 100% identity to the nucleic acid sequence of SEQ ID NO: 15, SEQ ID NO: 38, SEQ ID NO: 40, SEQ ID NO: 42 or SEQ ID NO: 44, SEQ ID NO: 24, SEQ ID NO: 201, SEQ ID NO: 206, SEQ ID NO: 211, SEQ ID NO: 216, SEQ ID NO: 221, SEQ ID NO: 204, SEQ ID NO: 209, SEQ ID NO: 214, SEQ ID NO: 219, or SEQ ID NO: 224,
optionally wherein the second polynucleotide comprises:
(a) a nucleotide sequence having at least 85%, 90%, 95%, 96%, 97%, 98%, 99% or 100% identity to the sequence of SEQ ID NO: 221;
(b) the nucleotide sequence of SEQ ID NO: 221; or
(c) the nucleotide sequence of SEQ ID NO: 224 which consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 29.
   E93. The LNP composition of any one of embodiments 49-92, wherein the GM-CSF molecule comprises a half-life extender, e.g., a protein (or fragment thereof) that binds to a serum protein such as albumin, IgG, FcRn or transferrin.
   E94. The LNP composition of embodiment 93, wherein the half-life extender comprises albumin or a fragment thereof, or an Fc domain of an antibody molecule (e.g., an Fc domain with enhanced FcRn binding).
   E95. The LNP composition of embodiment 93 or 94, wherein the half-life extender is albumin, or a fragment thereof.
   E96. The LNP composition of any one of embodiments 93-95, wherein the half-life extender is albumin, e.g., human serum albumin (HSA), mouse serum albumin (MSA), cyno serum albumin (CSA) or rat serum albumin (RSA).
   E97. The LNP composition of embodiment 96, wherein the albumin is HSA comprising an amino acid sequence having at least 85%, 90%, 95%, 96%, 97%, 98%, 99% or 100% identity to the amino acid sequence of SEQ ID NO: 8.
   E98. The LNP composition of any one of embodiments 93-97, wherein the GM-CSF molecule comprising HSA comprises an amino acid sequence having at least 85%, 90%, 95%, 96%, 97%, 98%, 99% or 100% identity to the amino acid sequence of SEQ ID NO: 16 or SEQ ID NO: 220.
   E99. The LNP composition of any one of embodiments 93-97, wherein the GM-CSF molecule comprising HSA is encoded by a nucleotide sequence having at least 85%, 90%, 95%, 96%, 97%, 98%, 99% or 100% identity to the nucleic acid sequence of SEQ ID NO: 24, SEQ ID NO: 221, or SEQ ID NO: 224.
   E100. The LNP composition of any one of embodiments 49-99, wherein the GM-CSF molecule further comprises a targeting moiety, e.g., a dendritic cell targeting moiety, or a tissue-specific targeting moiety.
   E101. The LNP composition of embodiment 100, wherein the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E102. The LNP composition of any one of the preceding embodiments, wherein the first and second polynucleotides are formulated at an (a):(b) mass ratio of 10:1, 8:1, 6:1, 4:1, 3:1, 2:1, 1.5:1, or 1:1.
   E103. The LNP composition of any one of embodiments 49-101, wherein the first and second polynucleotides are formulated at an (a):(b) mass ratio of 1:1.5, 1:2, 1:3, 1:4, 1:6, 1:8, or 1:10.
   E104. The LNP composition of any one of the preceding embodiments, wherein the LNP composition increases the level and/or activity of T regulatory cells and/or suppressor T cells, e.g., as determined by an assay in a sample (e.g., a sample from a subject).
   E105. The LNP composition of embodiment 104, wherein the T regulatory cells comprise FoxP3+ expressing and/or CD25+ expressing T regulatory cells.
   E106. The LNP composition of embodiment 104 or 105, wherein the T regulatory cells are CD4+ and/or CD8+ T regulatory cells.
   E107. The LNP composition of any one of embodiments 104-106, wherein the increase in level and/or activity of T regulatory cells is compared to level and/or activity of T regulatory cells in an otherwise similar sample which is: not contacted with the LNP composition comprising (a) and (b); or contacted with a composition comprising only (a) or a composition comprising only (b).
   E108. The LNP composition of any one of embodiments 104-107, wherein the increase in level and/or activity of T regulatory cells occurs in vitro or in vivo.
   E109. The LNP composition of any one of embodiments 104-108, wherein the increase in level and/or activity of T regulatory cells comprises a one, or all or a combination (e.g., 2, 3, or all) of the following parameters:
(a) increased level of (e.g., number or proportion of) T regulatory cells (e.g., FoxP3+ T regulatory cells);
(b) increased activity of STAT5, e.g., STAT5 phosphorylation, in T regulatory cells (e.g., FoxP3+ T regulatory cells);
(c) increased activity or expression level of CTLA-4, TIGIT, ICOS and/or GITR in T regulatory cells (e.g., FoxP3+ T regulatory cells); and
(d) decreased activity or expression level of TGF beta and/or IL-10.
   E110. The LNP composition of embodiment 109, wherein the LNP composition increases the level of (e.g., number or proportion of) FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1-3, 8 or 11.
   E111. The LNP composition of embodiment 110, wherein the increase in the level of Fox P3+ T regulatory cells is compared to an otherwise similar population of cells not contacted with a composition comprising IL-2 and GM-CSF.
   E112. The LNP composition of embodiment 109, wherein the LNP composition increases in the activity of STAT5 (e.g., STAT5 phosphorylation) in FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1.
   E113. The LNP composition of embodiment 112, wherein the increase in activity of STAT5 is compared to the activity of STAT5 in FoxP3− cells or Natural Killer cells.
   E114. The LNP composition of embodiment 109, wherein the LNP composition increases in the activity and/or expression level of one or more (e.g., two, three, or all) of CTLA-4, TIGIT, ICOS and/or GITR in T regulatory cells (e.g., FoxP3+ T regulatory cells), e.g., a 1.5 to 10 fold (e.g., 2 to 8 fold, 3 to 7 fold, 4 to 6 fold, 1.5 to 10 fold, 1.5 to 8 fold, 1.5 to 6 fold, 1.5 to 4 fold, 8 to 10 fold, 6 to 10 fold, or 4 to 10 fold) increase, as measured by an assay in Example 2.
   E115. The LNP composition of embodiment 114, wherein the increase in activity and/or expression level of one or more (e.g., two, three, or all) of CTLA-4, TIGIT, ICOS and/or GITR in T regulatory cells is compared to an otherwise similar population of cells not contacted with a composition comprising IL-2 and GM-CSF.
   E116. The LNP composition of any one of the preceding embodiments, wherein the composition increases T regulatory cells (e.g., CD25+ T regulatory cells) as compared to type 1 T helper cells (Tₕ1) cells; type 2 T helper cells (Tₕ2) cells; type 17 T helper cells (Tₕ17) cells and/or CD8+ T conventional cells (T con).
   E117. The LNP composition of embodiment 116, wherein the increase in level and/or activity of suppressor T cells is compared to level and/or activity of suppressor T cells in an otherwise similar sample which is: not contacted with the composition comprising (a) and (b); or contacted with a composition comprising only (a) or a composition comprising only (b).
   E118. The LNP composition of embodiment 117, wherein the increase in level and/or activity of suppressor T cells occurs in vitro or in vivo.
   E119. The LNP composition of embodiment 117 or 118, wherein the increase in level and/or activity of suppressor T cells comprises one or both of the following parameters:
(a) increased activity or expression level of Lag 3; and/or
(b) increased activity or expression level of CD94b.
   E120. The LNP composition of any one of the preceding embodiments, wherein the first polynucleotide, the second polynucleotide, or both, comprises at least one chemical modification.
   E121. The LNP composition of embodiment 120, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   E122. The LNP composition of embodiment 121, wherein the chemical modification is selected from the group consisting of pseudouridine, N1-methylpseudouridine, 5-methylcytosine, 5-methoxyuridine, and a combination thereof.
   E123. The LNP composition of embodiment 122, wherein the chemical modification is N1-methylpseudouridine.
   E124. The LNP composition of any one of the preceding embodiments, wherein each mRNA in the lipid nanoparticle comprises fully modified N1-methylpseudouridine.
   E125. The LNP composition of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
   E126. The LNP composition of embodiment 125, wherein the ionizable lipid comprises an amino lipid.
   E127. The LNP composition of embodiment 125 or 126, wherein the ionizable lipid comprises a compound of any of Formulae (I I), (I IA), (I IB), (I II), (I Ha), (I IIb), (I IIc), (I IId), (I He), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIc), (I VIIId), (I IX), (I IXa1), (I IXa2), (I IXa3), (I IXa4), (I IXa5), (I IXa6), (I IXa7), or (I IXa8).
   E128. The LNP composition of any one of embodiments 125-127, wherein the ionizable lipid comprises a compound of Formula (I I).
   E129. The LNP composition of any one of embodiments 125-128, wherein the ionizable lipid comprises Compound 18 or Compound 25.
   E130. The LNP composition of any one of embodiments 125-129, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.
   E131. The LNP composition of embodiment E130, wherein the phospholipid is DSPC.
   E132. The LNP composition of embodiment E130, wherein the phospholipid is DMPE.
   E133. The LNP composition of embodiment E130, wherein the phospholipid is Compound H-409.
   E134. The LNP composition of any one of embodiments 125-133, wherein the structural lipid is selected from β-sitosterol and cholesterol.
   E135. The LNP composition of any one of embodiments 125-134, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
   E136. The LNP composition of embodiment 135, wherein the PEG lipid is selected from the group consisting of PEG-c-DOMG, PEG-DMG, PEG-DLPE, PEG-DMPE, PEG-DPPC and PEG-DSPE lipid.
   E137. The LNP composition of embodiment 136, wherein the PEG-lipid is PEG-DMG.
   E138. The LNP composition of any one of embodiments 125-137, wherein the PEG lipid comprises a compound selected from the group consisting of Compound P415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.
   E139. The LNP composition of embodiment 138, wherein the PEG lipid comprises a compound selected from the group consisting of Compound P-428, Compound PL-16, Compound PL-17, Compound PL-18, Compound PL-19, Compound PL-1, and Compound PL-2.
   E140. The LNP composition of embodiment 138, wherein the PEG lipid is Compound P-428.
   E141. The LNP composition of any one of embodiments 125-140, wherein the LNP comprises a molar ratio of about 20-60% ionizable lipid:5-25% phospholipid:25-55% cholesterol; and 0.5-15% PEG lipid.
   E142. The LNP composition of embodiment 141, wherein the LNP comprises a molar ratio of about 50% ionizable lipid:about 10% phospholipid:about 38.5% cholesterol; and about 1.5% PEG lipid.
   E143. The LNP composition of embodiment 141 or 142, wherein the LNP comprises a molar ratio of about 49.83% ionizable lipid:about 9.83% phospholipid:about 30.33% cholesterol; and about 2.0% PEG lipid.
   E144. The LNP composition of embodiment 141 or 142, wherein the LNP comprises a molar ratio of about 47.5% ionizable lipid:about 10.5% phospholipid:about 39% cholesterol; and about 3% PEG lipid.
   E145. The LNP composition of any one of embodiments 141-143, wherein the ionizable lipid comprises a compound of any of Formulae (I I), (I IA), (I IB), (I II), (I Ha), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIII), (I IX), (I IXa1), (I IXa2), (I IXa3), (I IXa4), (I IXa5), (I IXa6), (I IXa7), or (I IXa8).
   E146. The LNP composition of embodiment 145, wherein the ionizable lipid comprises a compound of Formula (I I).
   E147. The LNP composition of embodiment 145 or 146, wherein the ionizable lipid comprises Compound 18 or Compound 25.
   E148. The LNP composition of any one of embodiments 141-147, wherein the PEG lipid is PEG-DMG or Compound P-428.
   E149. The LNP composition of any one of the preceding embodiments, which is formulated for intravenous, subcutaneous, intramuscular, intranasal, intraocular, rectal or oral delivery.
   E150. The LNP composition of any one of the preceding embodiments, further comprising a pharmaceutically acceptable carrier or excipient.
   E151. A pharmaceutical composition comprising the lipid nanoparticle, of any one of embodiments 1 to 150.
   E152. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, in the treatment and/or prevention of a disease associated with an aberrant T regulatory cell function in a subject.
   E153. A method of treating and/or preventing a disease associated with an aberrant T regulatory cell function in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.
   E154. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for inhibiting an immune response in a subject.
   E155. A method of inhibiting an immune response in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.
   E156. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for stimulating T regulatory cells in a subject.
   E157. A method of stimulating T regulatory cells in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.
   E158. An LNP composition of any one of embodiments 1 to 150, or the pharmaceutical composition of embodiment 151, for use in the treatment of a disease associated with an aberrant T regulatory cell function in a subject.
   E159. A method of treating and/or preventing a disease associated with an aberrant T regulatory cell function in a subject, comprising administering to the subject an effective amount of the LNP composition of any one of embodiments 1 to 150, or the pharmaceutical composition of embodiment 151.
   E160. The LNP composition for use, or the method of any one of embodiments 152-158, wherein the dose, e.g., effective dose, of the first polynucleotide encoding the IL-2 molecule in the lipid nanoparticle is at least 10%, 20%, 30%, 40%, 50%, 60%, 70%, 80% or 95% lesser than the dose of a naturally occurring, or recombinant IL-2, e.g., in an otherwise similar LNP.
   E161. The LNP composition for use, or the method of any one of embodiments 152 to 160, wherein the LNP composition is administered by a route of administration chosen from: subcutaneous, intramuscular, intravenous, oral, intranasal, intraocular, or rectal.
   E162. The LNP composition for use, or method of any one of embodiments 152 to 161, wherein the LNP composition is administered subcutaneously.
   E163. The LNP composition for use, or method of any one of embodiments 152 to 157 or 160 to 162, wherein the first LNP and the second LNP are administered sequentially or simultaneously.
   E163. The LNP composition for use, or method of any one of embodiments 152 to 157 or 160 to 162, wherein the first LNP and the second LNP are administered sequentially or simultaneously.
   E164. The LNP composition for use, or method of any one of embodiments 152 to 157 or 160 to 163, wherein the first LNP and the second LNP are administered in the same or in separate compositions.
   E165. The LNP composition for use, or method of any one of embodiments 152 to 157 or 160 to 164, wherein the first LNP comprising the first polynucleotide encoding the IL-2 molecule is administered first and the second LNP comprising the second polynucleotide encoding the GM-CSF molecule is administered second.
   E166. The LNP composition for use, or method of any one of embodiments 152 to 157 or 160 to 165, wherein the first LNP comprising the first polynucleotide encoding the IL-2 molecule is administered at least 1 day, 2 days, 3 days, 4 days, 5 days, 6 days, 7 days, 8 days, 9 days, or 10 days (e.g., 7 days), before administration of the second LNP comprising the second polynucleotide encoding the GM-CSF molecule.
   E167. The LNP composition for use, or method of any one of embodiments 152 to 157 or 160 to 166, wherein the first LNP comprising the first polynucleotide encoding the IL-2 molecule is administered at least 1 week, 2 weeks, 3 weeks, 4 weeks, 5 weeks, or 6 weeks, before administration of the second LNP comprising the second polynucleotide encoding the GM-CSF molecule.
   E168. The LNP composition for use, or method of any one of embodiments 152 to 157 or 160 to 167, wherein the first LNP comprising the first polynucleotide encoding the IL-2 molecule is administered second and the second LNP comprising the second polynucleotide encoding the GM-CSF molecule is administered first.
   E169. The LNP composition for use, or method of any one of embodiments 152 to 157 or 160 to 168, wherein the first LNP comprising the first polynucleotide encoding the IL-2 molecule is administered at least 1 day, 2 days, 3 days, 4 days, 5 days, 6 days, 7 days, 8 days, 9 days, or 10 days (e.g., 7 days), after administration of the second LNP comprising the second polynucleotide encoding the GM-CSF molecule.
   E170. The LNP composition for use, or method of any one of embodiments 152 to 157 or 160 to 169, wherein the first LNP comprising the first polynucleotide encoding the IL-2 molecule is administered at least 1 week, 2 weeks, 3 weeks, 4 weeks, 5 weeks, or 6 weeks, after administration of the second LNP comprising the second polynucleotide encoding the GM-CSF molecule.
   E171. The LNP composition for use, or method of any one of embodiments 152 to 157 or 160 to 170, comprising a dosing interval, wherein the dosing interval comprises:
(a) an initial dose of the first LNP and one or more subsequent doses of the second LNP;
(b) an initial dose of the second LNP and one or more subsequent doses of the first LNP;
(c) an initial dose of the first LNP followed by one or more subsequent doses of a combination of the first LNP and the second LNP;
(d) an initial dose of the second LNP followed by one or more subsequent doses of a combination of the first LNP and the second LNP; and/or
(e) one or more doses of the initial dose of the first LNP or the second LNP.
   E172. The LNP composition for use, or the method of embodiment 171, wherein the dosing interval comprises an initial dose of the first LNP and one or more subsequent doses of the second LNP.
   E173. The LNP composition for use, or the method of embodiment 171 or 172, wherein the dosing interval comprises an initial dose of the second LNP and one or more subsequent doses of the first LNP.
   E174. The LNP composition for use, or the method of any one of embodiments 171 to 173, wherein the dosing interval comprises an initial dose of the first LNP followed by one or more subsequent doses of a combination of the first LNP and the second LNP.
   E175. The LNP composition for use, or the method of any one of embodiments 171 to 174, wherein the dosing interval comprises an initial dose of the second LNP followed by one or more subsequent doses of a combination of the first LNP and the second LNP.
   E176. The LNP composition for use, or the method of any one of embodiments 171 to 175, wherein the dosing interval comprises an initial dose of the second LNP followed by one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, 1-5 doses) of a combination of the first LNP and the second LNP.
   E177. The LNP composition for use, or the method of any one of embodiments 171 to 176, wherein the dosing interval is performed over at least 1 week, 2 weeks, 3 weeks, or 4 weeks.
   E178. The LNP composition for use, or the method of any one of embodiments 171 to 177, wherein the one or more subsequent doses of the combination of the first LNP and second LNP are administered, e.g., at least 5-20 days, 5-19 days, 5-18 days, 5-17 days, 5-16 days, 5-15 days, 5-14 days, 5-13 days, 5-12 days, 5-11 days, 5-10 days, 5-9 days, 5-8 days, 5-7 days, 5-6 days, 6-20 days, 7-20 days, 8-20 days, 9-20 days, 10-20 days, 11-20 days, 12-20 days, 13-20 days, 14-20 days, 15-20 days, 16-20 days, 17-20 days, 18-20 days, or 19-20 days, e.g., 7-14 days, after administration of the initial dose of the second LNP.
   E179. The LNP composition for use, or the method of any one of embodiments 171 to 178, wherein the dosing interval is repeated at least 1 time, at least 2 times, at least 3 times, at least 4 times, at least 5 times, at least 6 times, at least 7 times, at least 8 times, at least 9 times, or at least times.
   E180. The LNP composition for use, or the method of any one of embodiments 171 to 179, wherein the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years or 5 years.
   E181. The LNP composition for use, or method of any one of embodiments 152 to 130, wherein an initial dose of an LNP (e.g., an LNP described herein) is at least 10%, 20%, 25%, 30%, 35%, 40%, 45%, 50%, 55%, 60%, 65%, 70%, 75%, 80%, 85%, or 90% lower than a subsequent dose of an LNP (e.g., the same LNP).
   E182. The LNP composition for use, or the method of embodiment 181, wherein the initial dose of the first LNP comprising IL-2 is at least 10%, 20%, 25%, 30%, 35%, 40%, 45%, 50%, 55%, 60%, 65%, 70%, 75%, 80%, 85%, or 90% lower than the subsequent dose of the first LNP comprising IL-2 (e.g., administered alone or in combination with the second LNP comprising GM-CSF).
   E183. The LNP composition for use, or the method of embodiment 181 or 182, wherein the initial dose of the second LNP comprising the second polynucleotide encoding the GM-CSF is at least 10%, 20%, 25%, 30%, 35%, 40%, 45%, 50%, 55%, 60%, 65%, 70%, 75%, 80%, 85%, or 90% lower than the subsequent dose of the second LNP comprising the second polynucleotide encoding the GM-CSF (e.g., administered alone or in combination with the first LNP comprising IL-2).
   E184. A composition comprising a lipid nanoparticle (LNP) comprising a first polynucleotide encoding an IL-2 molecule and a second polynucleotide encoding a GM-CSF molecule for use, in the treatment and/or prevention of a disease associated with an aberrant T regulatory cell function in a subject.
   E185. A method of treating and/or preventing a disease associated with an aberrant T regulatory cell function in a subject, comprising administering to the subject an effective amount of a lipid nanoparticle (LNP) comprising a first polynucleotide encoding an IL-2 molecule and a second polynucleotide encoding a GM-CSF molecule.
   E186. The LNP composition for use, or the method of embodiment 184 or 185, wherein prior to the administration of the LNP comprising the first polynucleotide encoding the IL-2 molecule and the second polynucleotide encoding the GM-CSF molecule, a different LNP comprising a third polynucleotide encoding a GM-CSF molecule is administered to the subject.
   E187. The LNP composition for use, or the method of embodiment 186, wherein the LNP comprising a third polynucleotide encoding the GM-CSF molecule does not comprise a polynucleotide encoding an IL-2 molecule.
   E188. The LNP composition for use, or the method of embodiment 186 or 187, wherein the second polynucleotide encoding GM-CSF and the third polynucleotide encoding GM-CSF comprise the same or substantially the same polynucleotide sequence.
   E189. The LNP composition for use, or method of any one of embodiments 186 to 188, wherein the different LNP comprising a third polynucleotide encoding a GM-CSF molecule is administered at least 1 day, 2 days, 3 days, 4 days, 5 days, 6 days, 7 days, 8 days, 9 days, or 10 days (e.g., 7 days), prior to the administration of the LNP comprising a first polynucleotide encoding an IL-2 molecule and a second polynucleotide encoding a GM-CSF molecule.
   E190. The LNP composition for use, or method of any one of embodiments 186 to 189, wherein the different LNP comprising a third polynucleotide encoding a GM-CSF molecule is administered at least 1 week, 2 weeks, 3 weeks, 4 weeks, 5 weeks, or 6 weeks prior to the administration of the LNP comprising a first polynucleotide encoding an IL-2 molecule and a second polynucleotide encoding a GM-CSF molecule.
   E191. The LNP composition for use, or method of any one of embodiments 152 to 190, wherein the first and second polynucleotides are formulated at an (a):(b) mass ratio of:
(i) 10:1, 8:1, 6:1, 4:1, 3:1, 2:1, 1.5:1, or 1:1; or
(ii) 1:1, 1:1.5, 1:2, 1:3, 1:4, 1:6, 1:8, or 1:10.
   E192. The LNP composition for use, or method of any one of embodiments 186 to 191, wherein the dose, e.g., effective dose, of the GM-CSF molecule in the LNP comprising the third polynucleotide encoding GM-CSF is at least 10%, 20%, 30%, 40%, 50%, 60%, 70%, 80% or 95% lesser than the dose, e.g., effective dose, of the GM-CSF molecule in the LNP comprising the first and second polynucleotides.
   E193. The LNP composition for use, or the method of any one of embodiments 152 to 192, wherein the dose, e.g., effective dose, of the first polynucleotide encoding the IL-2 molecule in the lipid nanoparticle is at least 10%, 20%, 30%, 40%, 50%, 60%, 70%, 80% or 95% lesser than the dose of a naturally occurring, or recombinant IL-2, e.g., in an otherwise similar LNP.
   E194. The LNP composition for use, or the method of any one of embodiments 152 to 193, wherein the LNP composition is administered by a route of administration chosen from: subcutaneous, intramuscular, intravenous, oral, intraocular or rectal.
   E195. The LNP composition for use, or method of any one of embodiments 152 to 194, wherein the composition is administered subcutaneously.
   E196. The LNP composition for use, or the method of any one of embodiments 152 to 195, wherein the disease associated with an aberrant T regulatory cell function is an autoimmune disease, or a disease with hyper-activated immune function.
   E197. The LNP composition for use, or the method of embodiment 196, wherein the autoimmune disease is chosen from: rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)); multiple sclerosis; autoimmune hepatitis (e.g., Type 1 or Type 2); primary biliary cholangitis; organ transplant associated rejection; myasthenia gravis; Parkinsons's Disease; Alzheimer's Disease; amyotrophic lateral sclerosis; psoriasis; or polymyositis (also known as dermatomyositis).
   E198. The LNP composition for use, or the method of any one of embodiments 152 to 197, wherein the subject is a mammal, e.g., a human.
   E199. The LNP composition for use, or the method, of any one of embodiments 152 to 198, wherein the composition or method results in an increase in the level and/or activity of T regulatory cells and/or suppressor T cells in a sample or a subject.
   E200. The LNP composition for use, or the method, of embodiment 199, wherein the T regulatory cells comprise FoxP3+ expressing and/or CD25+ expressing T regulatory cells.
   E200. The LNP composition for use, or the method, of embodiment 199 or 200, wherein the T regulatory cells are CD4+ and/or CD8+ T regulatory cells.
   E202. The LNP composition for use, or the method, of any one of embodiments 199 to 201, wherein the increase in level and/or activity of T regulatory cells is compared to level and/or activity of T regulatory cells in an otherwise similar sample or subject: not contacted with a composition comprising the first and second LNPs.
   E203. The LNP composition for use, or the method, of any one of embodiments 199 to 202, wherein the increase in level and/or activity of T regulatory cells occurs in vitro or in vivo.
   E204. The LNP composition for use, or the method, of any one of embodiments 199 to 203, wherein the increase in level and/or activity of T regulatory cells comprises one, or all or a combination (e.g., 2, 3, or all) of the following parameters:
(a) increased level of (e.g., number or proportion of) T regulatory cells (e.g., FoxP3+ T regulatory cells);
(b) increased activity of STAT5, e.g., STAT5 phosphorylation, in T regulatory cells (e.g., FoxP3+ T regulatory cells);
(c) increased activity or expression level of CTLA-4, TIGIT, ICOS and/or GITR in T regulatory cells (e.g., FoxP3+ T regulatory cells); and
(d) decreased activity or expression level of TGF beta and/or IL-10.
   E205. The LNP composition for use, or the method, of embodiment 204, wherein the composition or method results in an increase in the level of (e.g., number or proportion of) FoxP3+ T regulatory cells, e.g., a 1.5 to 5 fold increase, as measured by an assay in Examples 1-3, 8 or 11.
   E206. The LNP composition for use, or the method, of embodiment 205, wherein the increase in the level of Fox P3+ T regulatory cells is compared to an otherwise similar population of cells not contacted with a composition comprising IL-2 and GM-CSF.
   E207. The LNP composition for use, or the method, of embodiment 204, wherein the composition or method results in an increase in the activity of STAT5 (e.g., STAT5 phosphorylation) in FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1.
   E208. The LNP composition for use, or the method, of embodiment 207, wherein the increase in activity of STAT5 is compared to the activity of STAT5 in FoxP3− cells or Natural Killer cells.
   E209. The LNP composition for use, or the method, of any one of embodiments 152 to 208, wherein the composition results in an increase in the activity and/or expression level of CTLA-4, TIGIT, ICOS and/or GITR in T regulatory cells (e.g., FoxP3+ T regulatory cells), e.g., a 1.5 to fold (e.g., 2 to 8 fold, 3 to 7 fold, 4 to 6 fold, 1.5 to 10 fold, 1.5 to 8 fold, 1.5 to 6 fold, 1.5 to 4 fold, 8 to 10 fold, 6 to 10 fold, or 4 to 10 fold) increase, as measured by an assay in Example 2.
   E210. The LNP composition for use, or the method, of embodiment 209, wherein the increase in activity and/or expression level of CTLA-4, TIGIT, ICOS and/or GITR in T regulatory cells is compared to an otherwise similar population of cells not contacted with a composition comprising IL-2 and GM-CSF.
   E211. The LNP composition for use, or the method of any of embodiments 152 to 210, wherein the composition results in a preferential increase of T regulatory cells (e.g., CD25+ T regulatory cells) as compared to type 1 T helper cells (Tₕ1) cells; type 2 T helper cells (Tₕ2) cells; type 17 T helper cells (Tₕ17) cells, and/or CD8+ T conventional cells (T con).
   E212. The LNP composition for use, or the method, of embodiment 211, wherein the increase in level and/or activity of suppressor T cells is compared to level and/or activity of suppressor T cells in an otherwise similar sample or subject: not contacted with the composition comprising the first and second LNPs.
   E213. The LNP composition for use, or the method, of embodiment 211 or 212, wherein the increase in level and/or activity of suppressor T cells occurs in vitro or in vivo.
   E214. The LNP composition for use, or the method, of embodiment 163, wherein the increase in level and/or activity of suppressor T cells comprises one or both of the following parameters:
(a) increased activity or expression level of Lag 3; and/or
(b) increased activity or expression level of CD94b.
   E215. A lipid nanoparticle comprising a polynucleotide encoding a molecule that stimulates T regulatory cells (e.g., an IL-2 molecule) for use, in the treatment of a disease associated with an aberrant T regulatory cell function in a subject.
   E216. A method of treating and/or preventing a disease associated with an aberrant T regulatory cell function in a subject, comprising administering to the subject an effective amount of a lipid nanoparticle comprising a polynucleotide encoding a molecule that stimulates T regulatory cells (e.g., an IL-2 molecule).
   E217. The LNP composition for use of embodiment 215, or the method of embodiment 216, further comprising administration of a lipid nanoparticle comprising a polynucleotide encoding a GM-CSF molecule.
   E218. The LNP composition for use of embodiment 215 or 217, or the method of embodiment 216 or 217, wherein the molecule that stimulates T regulatory cells comprises an IL-2 molecule, or a molecule that binds to a receptor present on T regulatory cells.
   E219. A lipid nanoparticle (LNP) comprising a polynucleotide encoding a molecule that stimulates dendritic cells (e.g., a GM-CSF molecule) for use, in the treatment of a disease associated with an aberrant T regulatory cell function in a subject.
   E220. A method of treating and/or preventing a disease associated with an aberrant T regulatory cell function in a subject, comprising administering to a subject an effective amount of a lipid nanoparticle comprising a polynucleotide encoding molecule that stimulates dendritic cells (e.g., a GM-CSF molecule).
   E221. The LNP composition for use of embodiment 219, or the method of embodiment 220, further comprising administration of a lipid nanoparticle comprising a polynucleotide encoding an IL-2 molecule.
   E222. The LNP composition for use, or the method of any one of embodiments 219 to 221 wherein the molecule that stimulates dendritic cells comprises a molecule that stimulates, e.g., increases, the expression and/or level of TNFalpha, IL-10, CCL-2 and/or nitric oxide in dendritic cells.
   E223. The LNP composition for use, or the method of any one of embodiments 219 to 222, wherein the molecule that stimulates dendritic cells comprises a GM-CSF molecule.
   E224. The LNP composition for use, or the method of any one of embodiments 219 to 223, wherein the molecule that stimulates dendritic cells results in an increased level and/or activity of CD11b+ or CD11c+ dendritic cells.
   E225. The LNP composition for use, or the method of any one of embodiments 152 to 214, or 216 to 224, wherein administration of the LNP comprising the polynucleotide encoding the GM-CSF molecule results in a modulation of dendritic cell activity and/or modulation of expression and/or activity of myeloid cells in a sample from the subject.
   E226. The LNP composition for use, or the method of embodiment 225, wherein the sample has an increase in, e.g., increased number or proportion of, dendritic cells expressing CD11b and/or CD11c.
   E227. The LNP composition for use, or the method of embodiment 226, wherein the increase in DCs expressing CD11b (CD11b+ DCs) is at least 1.2-10 fold (e.g., at least 1.2, 1.5, 2, 3, 4, 5, 6, 7, 8, 9, or 10 fold), e.g., as compared to an otherwise similar sample not contacted with the LNP comprising the GM-CSF molecule, or contacted with a different LNP.
   E228. The LNP composition for use, or the method of embodiment 226, wherein the increase in DCs expressing CD11c (CD11c+ DCs) is at least 1.2-20 fold (e.g., at least 1.2, 1.5, 2, 3, 4, 5, 6, 7, 8, 9, 10, 11, 12, 13, 14, 15, 16, 17, 18, 19, or 20 fold), e.g., as compared to an otherwise similar sample not contacted with the LNP comprising the GM-CSF molecule, or contacted with a different LNP.
   E229. The LNP composition for use, or the method of any one of embodiments 225 to 228, wherein the sample has an increase in, e.g., increased number or proportion of, myeloid cells expressing CD11b, e.g., as compared to an otherwise similar sample not contacted with the LNP comprising the GM-CSF molecule, or contacted with a different LNP.
   E230. The LNP composition for use, or the method of any one of embodiments 152 to 218, or 221 to 229, wherein the IL-2 molecule comprises a naturally occurring IL-2 molecule, a fragment of a naturally occurring IL-2 molecule, or a variant thereof.
   E231. The LNP composition for use, or the method of embodiment 230, wherein the IL-2 molecule comprises a variant of a naturally occurring IL-2 molecule (e.g., an IL-2 variant, e.g., as described herein), or a fragment thereof.
   E232. The LNP composition for use, or the method of embodiment 231, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).
   E233. The LNP composition for use, or the method of embodiment 231 or 232, wherein the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.
   E234. The LNP composition for use, or the method of any one of embodiments 231 to 233, wherein the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.
   E235. The LNP composition for use, or the method of any one of embodiments 231 to 234, wherein the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.
   E236. The LNP composition for use, or the method of any one of embodiments 231 to 235, wherein the IL-2 variant comprises a mutation, e.g., substitution, at position 88 of the IL-2 polypeptide sequence, e.g., an N88D substitution.
   E237. The LNP composition for use, or the method of any one of embodiments 231 to 236, wherein the IL-2 variant comprises a mutation, e.g., substitution, at position 91 of the IL-2 polypeptide sequence, e.g., a V91K substitution.
   E238. The LNP composition for use, or the method of any one of embodiments 231 to 237, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 88 of the IL-2 polypeptide sequence, e.g., an N88D substitution.
   E239. The LNP composition for use, or the method of any one of embodiments 231 to 237, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 91 of the IL-2 polypeptide sequence, e.g., a V91K substitution.
   E240. The LNP composition for use, or the method of any one of embodiments 231 to 237, wherein the IL-2 variant comprises a mutation, e.g., substitution, at position 125 of the IL-2 polypeptide sequence, e.g., a C125S substitution.
   E241. The LNP composition for use, or the method of any one of embodiments 231 to 240, wherein the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E242. The LNP composition for use, or the method of embodiment 241, wherein the IL-2 molecule comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.
   E243. The LNP composition for use, or the method of any one of embodiments 152 to 218, or 221 to 242, wherein the first polynucleotide comprises a nucleotide sequence having at least 85%, 90%, 95%, 96%, 97%, 98%, 99% or 100% identity to the sequence of SEQ ID NO: 7.
   E244. The LNP composition for use, or the method of any one of embodiments 152 to 218, or 221 to 242, wherein the IL-2 molecule comprises a half-life extender, e.g., a protein (or fragment thereof) that binds to a serum protein such as albumin, IgG, FcRn or transferrin.
   E245. The LNP composition for use, or the method of embodiment 244, wherein the half-life extender comprises albumin or a fragment thereof, or an Fc domain of an antibody molecule (e.g., an Fc domain with enhanced FcRn binding).
   E246. The LNP composition for use, or the method of embodiment 245, wherein the half-life extender is albumin, or a fragment thereof.
   E247. The LNP composition for use, or the method of embodiment 245, wherein the half-life extender is albumin, e.g., human serum albumin (HSA), mouse serum albumin (MSA), cyno serum albumin (CSA) or rat serum albumin (RSA).
   E248. The LNP composition for use, or the method of embodiment 246, wherein the albumin is HSA comprising an amino acid sequence having at least 85%, 90%, 95%, 96%, 97%, 98%, 99% or 100% identity to the amino acid sequence of SEQ ID NO: 8.
   E249. The LNP composition for use, or the method of any one of embodiments 152 to 218, or 221 to 248, wherein the IL-2 molecule comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without a leader sequence.
   E250. The LNP composition for use, or the method of any one of embodiments 152 218, or 221 to 249, wherein the IL-2 molecule comprises the amino acid sequence of SEQ ID NO: 9, SEQ ID NO: 10, SEQ ID NO: 11, SEQ ID NO: 12 or SEQ ID NO: 13 with or without a leader sequence.
   E251. The LNP composition for use, or the method of embodiment 250, wherein the IL-2 molecule comprises the amino acid sequence of SEQ ID NO: 9.
   E252. The LNP composition for use, or the method of embodiment 250, wherein the IL-2 molecule comprises the amino acid sequence of SEQ ID NO: 10.
   E253. The LNP composition for use, or the method of embodiment 250, wherein the IL-2 molecule comprises the amino acid sequence of SEQ ID NO:11.
   E254. The LNP composition for use, or the method of embodiment 250, wherein the IL-2 molecule comprises the amino acid sequence of SEQ ID NO: 12.
   E255. The LNP composition for use, or the method of embodiment 250, wherein the IL-2 molecule comprises the amino acid sequence of SEQ ID NO: 13.
   E256. The LNP composition for use, or the method of any one of embodiments 152 to 218, 221 to 250, or 253, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 25 or SEQ ID NO: 36;
(b) the nucleotide sequence of SEQ ID NO: 25 or SEQ ID NO: 36;
(c) the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29; or
(d) the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.
   E257. The LNP composition for use, or the method of any one of embodiments 152 to 218, or 221 to 256, wherein the IL-2 molecule further comprises a targeting moiety, e.g., a T regulatory cell targeting moiety or a tissue-specific targeting moiety.
   E258. The LNP composition for use, or the method of embodiment 257, wherein the tissue-specific targeting moiety binds to ROS-CII, EDA, EDB, TnC A1, SyETP, GLUT-2, GD2, FAP, VCAM or MADCAM.
   E259. The LNP composition of any one of embodiments 256 to 258, wherein the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E260. The LNP composition for use, or the method of embodiment 259, wherein the T regulatory cell targeting moiety binds to a molecule present on a T regulatory cell.
   E261. The LNP composition for use, or the method of embodiment 259 or 260, wherein the T regulatory cell targeting moiety comprises an antibody molecule that binds to CTLA-4, GITR, TLR8, or Nrp1.
   E262. The LNP composition for use, or the method of any one of embodiments 259-261, wherein the T regulatory cell targeting moiety comprises an antibody molecule that binds to CTLA-4.
   E263. The LNP composition for use, or the method of embodiment 262, wherein the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17.
   E264. The LNP composition for use, or the method of embodiment 263, wherein the IL-2 molecule comprising the targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20.
   E265. The LNP composition for use, or the method of embodiment 263, wherein the IL-2 molecule comprising the targeting moiety comprises the amino acid sequence of SEQ ID NO: 18, SEQ ID NO: 19, or SEQ ID NO: 20.
   E266. The LNP composition for use, or the method of embodiment 263, wherein the IL-2 molecule comprising the targeting moiety is encoded by a nucleotide sequence having at least 85%, 90%, 95%, 96%, 97%, 98%, 99% or 100% identity to the nucleic acid sequence of SEQ ID NO: 21, SEQ ID NO:22, or SEQ ID NO: 23.
   E267. The LNP composition for use, or the method of any one of embodiments 152 to 214 or 217 to 229, wherein the GM-CSF molecule comprises a naturally occurring GM-CSF molecule, a fragment of a naturally occurring GM-CSF molecule, or a variant thereof.
   E268. The LNP composition for use, or the method of embodiment 267, wherein the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E269. The LNP composition for use, or the method of embodiment 267, wherein the GM-CSF molecule comprises the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.
   E270. The LNP composition for use, or the method of embodiment 267, wherein the second polynucleotide comprises a nucleotide sequence having at least 85%, 90%, 95%, 96%, 97%, 98%, 99% or 100% identity to the nucleic acid sequence of SEQ ID NO: 15, SEQ ID NO: 38, SEQ ID NO: 40, SEQ ID NO: 42, SEQ ID NO: 44, SEQ ID NO: 24, SEQ ID NO: 201, SEQ ID NO: 206, SEQ ID NO: 211, SEQ ID NO: 216, SEQ ID NO: 221, SEQ ID NO: 204, SEQ ID NO: 209, SEQ ID NO: 214, SEQ ID NO: 219, or SEQ ID NO: 224,
optionally wherein second polynucleotide comprises:
(a) a nucleotide sequence having at least 85%, 90%, 95%, 96%, 97%, 98%, 99% or 100% identity to the sequence of SEQ ID NO: 221;
(b) the nucleotide sequence of SEQ ID NO: 221; or
(c) the nucleotide sequence of SEQ ID NO: 224 which consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 29.
   E271. The LNP composition for use, or the method of any one of embodiments 152 to 214 or 217 to 229, or 267 to 270, wherein the GM-CSF molecule comprises a half-life extender, e.g., a protein (or fragment thereof) that binds to a serum protein such as albumin, IgG, FcRn or transferrin.
   E272. The LNP composition for use, or the method of embodiment 271, wherein the half-life extender comprises albumin or a fragment thereof, or an Fc domain of an antibody molecule (e.g., an Fc domain with enhanced FcRn binding).
   E273. The LNP composition for use, or the method of embodiment 271 or 272, wherein the half-life extender is albumin, or a fragment thereof.
   E274. The LNP composition for use, or the method of any one of embodiments 271 to 273, wherein the half-life extender is albumin, e.g., human serum albumin (HSA), mouse serum albumin (MSA), cyno serum albumin (CSA) or rat serum albumin (RSA).
   E275. The LNP composition for use, or the method of embodiment 274, wherein the albumin is HSA comprising an amino acid sequence having at least 85%, 90%, 95%, 96%, 97%, 98%, 99% or 100% identity to the amino acid sequence of SEQ ID NO:8.
   E276. The LNP composition for use, or the method of embodiment 274, wherein the albumin is HSA comprising the amino acid sequence of SEQ ID NO:8.
   E277. The LNP composition for use, or the method of any one of embodiments 152 to 214 or 217 to 229, or 267 to 276, wherein the GM-CSF molecule further comprises a targeting moiety, e.g., a dendritic cell targeting moiety, or a tissue-specific targeting moiety.
   E278. The LNP composition for use, or the method of embodiment 277, wherein the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.
   E279. The LNP composition for use, or the method of any one of embodiments 152 to 278, wherein the first polynucleotide, the second polynucleotide, or both, comprises at least one chemical modification.
   E280. The LNP composition for use, or the method of embodiment 279, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. E281. The LNP composition for use, or the method of embodiment 280, wherein the chemical modification is selected from the group consisting of pseudouridine, N1-methylpseudouridine, 5-methylcytosine, 5-methoxyuridine, and a combination thereof.
   E282. The LNP composition for use, or the method of embodiment 281, wherein the chemical modification is N1-methylpseudouridine.
   E283. The LNP composition for use, or the method of any one of embodiments 152 to 282, wherein each mRNA in the lipid nanoparticle comprises fully modified N1-methylpseudouridine.
   E284. The LNP composition for use, or the method of any one of embodiments 152 to 283, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
   E285. The LNP composition for use, or the method of embodiment 284, wherein the ionizable lipid comprises an amino lipid.
   E286. The LNP composition for use, or the method of embodiment 284 or 285, wherein the ionizable lipid comprises a compound of any of Formulae (I I), (I IA), (I IB), (I II), (I Ia), (I IIb), (I IIc), (I IId), (I IIe), (I IIf), (I IIg), (I III), (I VI), (I VI-a), (I VII), (I VIII), (I VIIa), (I VIII), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).
   E287. The LNP composition for use, or the method of any one of embodiments 284 to 286, wherein the ionizable lipid comprises a compound of Formula (I I).
   E288. The LNP composition for use, or the method of any one of embodiments 284 to 287, wherein the ionizable lipid comprises Compound 18 or Compound 25.
   E289. The LNP composition for use, or the method of any one of embodiments 284 to 288, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.
   E290. The LNP composition for use, or the method of embodiment 289, wherein the phospholipid is DSPC.
   E291. The LNP composition for use, or the method of embodiment 290, wherein the phospholipid is DMPE.
   E292. The LNP composition for use, or the method of embodiment 290, wherein the phospholipid is Compound H-409.
   E293. The LNP composition for use, or the method of any one of embodiments 286 to 292, wherein the structural lipid is β-sitosterol or cholesterol.
   E294. The LNP composition for use, or the method of any one of embodiments 286 to 293, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
   E295. The LNP composition for use, or the method of embodiment 294, wherein the PEG lipid is selected from the group consisting of PEG-c-DOMG, PEG-DMG, PEG-DLPE, PEG-DMPE, PEG-DPPC and PEG-DSPE lipid.
   E296. The LNP composition for use, or the method of embodiment 244, wherein the PEG-lipid is PEG-DMG.
   E297. The LNP composition for use, or the method of any one of embodiments 288 to 296, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.
   E298. The LNP composition for use, or the method of embodiment 297, wherein the PEG lipid comprises a compound selected from the group consisting of Compound P-428, Compound PL-16, Compound PL-17, Compound PL-18, Compound PL-19, Compound PL-1, and Compound PL-2.
   E299. The LNP composition for use, or the method of embodiment 298, wherein the PEG lipid is Compound P-428.
   E300. The LNP composition for use, or the method of any one of embodiments 286 to 299, wherein the LNP comprises a molar ratio of about 20-60% ionizable lipid:5-25% phospholipid: 25-55% cholesterol; and 0.5-15% PEG lipid.
   E301. The LNP composition for use, or the method of embodiment 300, wherein the LNP comprises a molar ratio of about 50% ionizable lipid:about 10% phospholipid:about 38.5% cholesterol; and about 1.5% PEG lipid.
   E302. The LNP composition for use, or the method of embodiment 300 or 301, wherein the LNP comprises a molar ratio of about 49.83% ionizable lipid:about 9.83% phospholipid:about 30.33% cholesterol; and about 2.0% PEG lipid.
   E303. The LNP composition for use, or the method of embodiment 300 or 301, wherein the LNP comprises a molar ratio of about 47.5% ionizable lipid:about 10.5% phospholipid:about 39% cholesterol; and about 3% PEG lipid.
   E304. The LNP composition for use, or the method of any one of embodiments 300-303, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).
   E305. The LNP composition for use, or the method of embodiment 304, wherein the ionizable lipid comprises a compound of Formula (I I).
   E306. The LNP composition for use, or the method of embodiment 304 or 305, wherein the ionizable lipid comprises Compound 18 or Compound 25.
   E307. The LNP composition for use, or the method of any one of embodiments 300-306, wherein the PEG lipid is PEG-DMG or Compound P-428.
   E308. The LNP composition for use, or the method of any one of embodiments 152 to 307, further comprising a pharmaceutically acceptable carrier or excipient.
   E309. The LNP composition for any one of embodiments 1-150, the LNP composition for use, or the method, of any one of embodiments 152 to 308, wherein the composition, method or composition for use results in a preferential increase in the level and/or activity of T regulatory cells compared to CD8+ T conventional cells (T con), in a sample or a subject.
   E310. The LNP composition, LNP composition for use, or the method, of embodiment E309, wherein the T regulatory cells comprise FoxP3+ expressing T regulatory cells.
   E311. The LNP composition, LNP composition for use, or the method, of embodiment E309, wherein the CD8+ T con cells comprise CD8+ CD25+ T cells.
   E312. The LNP composition, LNP composition for use, or the method, of any one of embodiments E309-E311, wherein the increase in level and/or activity of T regulatory cells occurs in vitro or in vivo.
   E313. The LNP composition, LNP composition for use, or the method, of any one of embodiments E309-E311, wherein the increase in level and/or activity of T regulatory cells is at least 2-10 fold higher compared to the level and/or activity of CD8+ T con cells, e.g., as measured in an assay described in Example 11.
   E314. A kit comprising a container comprising the lipid nanoparticle (LNP) composition of any one of embodiment 1 to 150, or the pharmaceutical composition of embodiment 151 or 308, and a package insert comprising instructions for administration of the lipid nanoparticle or pharmaceutical composition for treating or delaying a disease associated with aberrant T regulatory cell function in an individual.
   E315. The kit of embodiment 314, wherein the lipid nanoparticle composition comprises a pharmaceutically acceptable carrier.

### BRIEF DESCRIPTIONS OF THE DRAWINGS

FIG. 1 provides graphs depicting STAT5 phosphorylation (pSTAT5) in T cells within a pool of human PBMCs stimulated with various dilutions of the supernatant of HeLa cells transfected with an mRNA encoding HSA-IL-2 fusion proteins, as indicated. Phosphorylation of STAT5 was determined by flow cytometry.

FIG. 2 provides graphs depicting the extent of STAT5 phosphorylation in NK cells within a pool of human PBMCs stimulated with various dilutions of the supernatant of HeLa cells transfected with an mRNA encoding HSA-IL-2 fusion proteins, as indicated. Phosphorylation of STAT5 was determined by flow cytometry.

FIG. 3A provides a graph depicting the percentage (%) of CD4+ FoxP3+ Treg cells from the spleens of mice treated with lipid nanoparticle-formulated mRNA encoding MSA-mIL2, HSA-hsIL2.v5, or a control mRNA NTFIX-01 as indicated. Percentage of CD4+ FoxP3+ cells was determined by flow cytometry. FIG. 3B provides a graph depicting the expression level (fold-change) of various Treg activation markers on Tregs isolated from the spleens of mice treated with lipid nanoparticle-formulated mRNA encoding MSA-mIL2. Mice treated with a lipid nanoparticle-formulated control mRNA (NTFIX-01) was used as a comparator. Expression level of activation markers was determined by flow cytometry.

FIG. 4A provides a graph depicting the number of CD4+ FoxP3+ Treg cells from the spleens of mice treated with a 0.1 mg/kg dose of lipid nanoparticle-formulated mRNA encoding HSA-IL-2 fusion proteins or the NTFIX-01 control, as indicated. FIG. 4B provides a graph depicting the number of CD4+ FoxP3−Tbet+ Th1 cells from the serum of mice treated with a 0.1 mg/kg dose of lipid nanoparticle-formulated mRNA encoding HSA-IL-2 fusion proteins or the NTFIX-01 control, as indicated. FIG. 4C provides a graph depicting the expression level of Granzyme-B in 4 different subsets of NK cells from the serum of mice treated with lipid nanoparticle-formulated mRNA encoding HSA-IL-2 fusion proteins or the NTFIX-01 control, as indicated.

FIG. 5 provides graphs depicting the concentration of HSA-IL2 fusion protein (left panel), the percentage (%) of FoxP3+ cells from the CD4+ T cell compartment (center panel), and the percentage (%) of subsets of Tregs displaying variation in expression of CD25 and CD45RA (right panel) from the CD4+ cell compartment from peripheral blood of cynomolgus monkeys over time following a single sub cutaneous administration of lipid nanoparticle-formulated mRNA encoding HSA-IL2.

FIGS. 6A-6D provide graphs depicting the levels of immune cells in animals dosed with lipid nanoparticle-formulated mRNA encoding MSA-IL2 in a graft vs host disease (GVHD) model. Briefly, 50 million splenocytes plus 5 million CD4+ T cells from a C57BL/6 mice donor (B6) are transferred to the progeny of B6 crossed with DBA mice (F1) to result in a partial mismatch. Animals were dosed on day 1, 8 and 15 with lipid nanoparticle-formulated mRNA encoding MSA-IL2. FIG. 6A shows the absolute number of donor CD8 T cells in the spleen of animals treated as indicated. FIG. 6B shows the absolute number of B cells in the spleen of animals treated as indicated. FIG. 6C shows the percentage of peripheral blood CD8 T cells expressing Granzyme B. FIG. 6D shows the percentage of peripheral blood CD8 T cells expressing IFNg.

FIG. 7 provides a graph depicting the aggregate score of arthritis in a collage-induced rat arthritis model following weekly subcutaneous administration of a 0.025 mg/kg dose of lipid nanoparticle-formulated mRNA encoding an RSA-IL2 fusion protein. Rats treated with dexamethasone (DEX), anti-CD20, or PBS were used as comparators.

FIG. 8A provides a graph depicting the percentage (%) of FoxP3+ Treg cells from the CD4+ T cell compartment in the spleens of mice following treatment with a single dose (1×) of a lipid nanoparticle-formulated mRNA encoding GM-CSF at 0.1 mg/kg or 0.01 mg/kg, or following treatment with multiple doses (4×) at 0.01 mg/kg. Treatment of mice with PBS was used as a comparator. FIG. 8B provides a graph depicting the percentage (%) of FoxP3+ Treg cells from the CD4+ T cell compartment in the blood of mice following treatment in increasing doses of a lipid nanoparticle-formulated mRNA encoding an MSA-GM-CSF fusion protein or the NTFIX control, as indicated.

FIG. 9 provides graphs depicting the concentration of CSA-cynoGM-CSF fusion protein (left panel), the percentage (%) of FoxP3+ cells from the CD4+ cell compartment (center panel), and the percentage (%) of of subsets of Tregs displaying variation in expression of CD25 and CD45RA (right panel) from the CD4+ T cell compartment from blood of cynomolgus monkeys over time following a single administration of lipid nanoparticle-formulated mRNA encoding CSA-cynoGM-CSF.

FIG. 10 provides graphs depicting the percentage (%) of CD4+ Th1, Th2, Th17, or CD25+ Treg cells in mice treated intravenously with a 0.1 mg/kg dose of lipid nanoparticle-formulated mRNA encoding MSA-IL2, MSA-GMCSF, or a combination of both, as indicated. Mice treated with PBS were used as a comparator.

FIG. 11 provides a graph showing the fraction of T-bet+ CD4+ Th1 cells in the serum of mice over a 4 week window following weekly treatment of lipid-nanoparticle-formulated mRNA encoding MSA-IL2 alone, MSA-GMCSF, or administered a combination of both either simultaneously (combo) or sequentially (sequential).

FIG. 12 shows T regulatory cell expansion with administration of LNP formulated HSA-IL2 (wildtype 1L2). The graph shows % FoxP3+ cells in CD4+ T cells at various timepoints in animals administered with 0.01 mg per kg, 0.03 mg per kg or 0.10 mg per kg.

FIGS. 13A-13C provide graphs showing an activated phenotype in T regulatory cells with administration of LNP formulated HSA-IL2 (wildtype 1L2). FIG. 13A provides a graph depicting CD25 expression level (CD25 MFI) in CD25+ Foxp3+ CD4 T cells in animals dosed with the indicated doses of LNP formulated HSA-IL2 (wildtype 1L2). FIG. 13B provides a graph depicting FOXP3 expression (FOXP3 MFI) in CD25+ Foxp3+ CD4 T cells in animals dosed with the indicated doses of LNP formulated HSA-IL2 (wildtype 1L2). FIG. 13C provides a graph depicting the percent of CD45RA− CD45RO+; CD45RA+ CD45RO−; CD45RA+CD45RO−; CD45RA− CD45RO− T regulatory cells in animals dosed with the 0.1 mg per kg of LNP formulated HSA-IL2 (wildtype 1L2).

FIG. 14 provides a series of graphs depicting activation of T con cells with administration of LNP formulated HSA-IL2 (wildtype 1L2).

FIG. 15 provides a series of graphs depicting CD8 T cell activation with administration of LNP formulated HSA-IL2 (wildtype 1L2). The colors indicate different doses of LNP as shown in FIG. 12.

FIG. 16 provides graphs showing the levels of IFNgamma, IL-10, I1-5 or I1-6 in the plasma of animals dosed with LNP formulated HSA-IL2 (wildtype 1L2). The animals were administered with 0.01 mg per kg, 0.03 mg per kg or 0.10 mg per kg of the LNP.

FIG. 17 provides graphs showing the levels of plasma cytokines in animals dosed with LNP formulated HSA-IL2 (wildtype 1L2). The cytokines depicted are: IFNgamma, IL-10, IL-12p70, IL-17A, I1-5, I1-6, IL-8, MCP1, MIP1a, MIP1b, or TNF-alpha. The animals were administered with 0.01 mg per kg, 0.03 mg per kg or 0.10 mg per kg of the LNP.

FIGS. 18A-18C are graphs showing prolonged proliferation and preferential expansion of T regulatory cells with administration of LNP formulated HSA-IL2 (TM88). FIG. 18A is a graph showing the half-life of LNP formulated HSA-IL2 (wildtype) or LNP formulated HSA-IL2 (TM88) in non-human primates. FIG. 18B is a graph showing the percent of FOXP3+ cells in CD4+ T cells in non-human primates dosed with LNP formulated HSA-IL2 (wildtype) or LNP formulated HSA-IL2 (TM88). The LNP formulated HSA-IL2 (TM88) was dosed at 0.01 mg per kg, 0.03 mg per kg or 0.1 mg per kg. The LNP formulated HSA-IL2 (wildtype) was dosed at 0.03 mg per kg. FIG. 18C provides a series of graphs showing preferential expansion and activation of T regulatory cells over CD8+ T con cells in non-human primates dosed with LNP formulated HSA-IL2 (TM88). The LNP formulated HSA-IL2 (TM88) was dosed at 0.01 mg per kg (top graph), 0.03 mg per kg (middle graph) or 0.1 mg per kg (bottom graph). Each graph shows FOXP3+ cells in CD4+ T cells on the left y axis and CD25+ cells in CD8+ T cells right y-axis.

FIGS. 19A-19F are graphs showing delayed disease onset and slower disease progression in the MOG35-55 EAE mouse model treated subcutaneously with LNP formulated HSA-IL2 (TM88). FIG. 19A is a graph showing the “mean change body weight,” which is the percent change in body weight from Day 0). FIG. 19B is a graph showing the “mean clinical scores,” which is the average score for each group plotted for each day of the study (0=normal, no overt signs of disease; 1=tail paresis; 2=righting reflex impaired; 3=partial hind limb paralysis; 4=complete hind limb paralysis or absence of ambulation; 5=complete hind limb paralysis with front limb paresis, euthanasia required). FIG. 19C is a graph showing the “percent disease free,” which is the percent of mice that in each group that score 0 plotted for each day. FIG. 19D is a graph showing the “mean peak score,” which is the average of the highest scores achieved by each mouse in each group. FIG. 19E is a graph showing the “mean day onset,” which is the average of the first day each mouse in a group scores 1 or more. FIG. 19F is a graph showing the “disease intensity,” in which the sum total scores of each mouse over the period of the study are averaged for each for each group.

FIGS. 20A-20B are graphs showing the percentage (%) of of subsets of Tregs with or without CD25 and CD45RA (right panel) from the CD4+ T cell compartment from blood of cynomolgus monkeys over time following a single subcutaneous administration of lipid nanoparticle-formulated mRNA encoding CSA-cynoGM-CSF.

### DETAILED DESCRIPTION

Regulatory T cells (also known as T regulatory cells or T regs) are an important cell type in the maintenance of immune tolerance. The best-known type of regulatory T cells is a subset of CD4+ T cells defined by the expression of the transcription factor FOXP3. However, methods of stimulating and/or increasing the number of regulatory T cells in vivo are not well understood.

Accordingly, disclosed herein is a composition comprising immune modulating polypeptides encoding cytokines which can stimulate and/or increase the number of regulatory T cells in vivo or ex vivo. The present disclosure provides, inter alia, lipid nanoparticle (LNP) compositions comprising immune modulating polypeptides and uses thereof. The LNP compositions of the present disclosure comprise mRNA therapeutics encoding immune modulating polypeptides, e.g., interleukin 2 (IL-2) and/or granulocyte macrophage colony stimulating factor (GM-CSF). Also disclosed herein are methods of using an LNP composition comprising immune modulating polypeptides, e.g., IL-2 and/or GM-CSF, for treating and/or prophylaxis of a disease associated with an aberrant T regulatory cell function, or for inhibiting an immune response in a subject.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Antibody molecule: In one embodiment, antibody molecules can be used for targeting to desired cell types. As used herein, “antibody molecule” refers to a naturally occurring antibody, an engineered antibody, or a fragment thereof, e.g., an antigen binding portion of a naturally occurring antibody or an engineered antibody. An antibody molecule can include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Exemplary antibody molecules include, but are not limited to, humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies@); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Er vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

GM-CSF molecule: As used herein, the term “GM-CSF molecule” refers to a full length naturally-occurring GM-CSF (e.g., a mammalian GM-CSF, e.g., human GM-CSF, e.g., associated with GenBank Accession Number NM_000758), a fragment (e.g., a functional fragment) of GM-CSF, or a variant of GM-CSF having at least 80%, 85%, 90%, 95%, 96%, 97%, 98%, or 99% sequence identity to: a naturally-occurring wild type GM-CSF or a fragment (e.g., a functional fragment) thereof. In some embodiments, the GM-CSF molecule is a GM-CSF gene product, e.g., a GM-CSF polypeptide. In some embodiments, the variant, e.g., active variant, is a derivative, e.g., a mutant, of a wild type polypeptide. In some embodiments, the GM-CSF variant, e.g., active variant of GM-CSF, has at least 50%, 60%, 70%, 80%, 85%, 90°/a, 95%, 96%, 97%, 98%, 99% or 100% activity of wild type GM-CSF polypeptide.

IL-2 molecule: As used herein, the term “IL-2 molecule” refers to a full length naturally-occurring IL-2 (e.g., a mammalian IL-2, e.g., human IL-2, e.g., associated with GenBank Accession Number NM_000586), a fragment (e.g., a functional fragment) of IL-2, or a variant of IL-2 having at least 80%, 85%, 90%, 95%, 96%, 97%, 98%, or 99% sequence identity to: a naturally-occurring wildtype IL-2 or a fragment (e.g., functional fragment) thereof. In some embodiments, the IL-2 molecule is an IL-2 gene product, e.g., an IL-2 polypeptide. In some embodiments, the variant, e.g., active variant, is a derivative, e.g., a mutant, of a wild type polypeptide. In some embodiments, the IL-2 variant, e.g., active variant of IL-2, has at least 50%, 60%, 70%, 80%, 85%, 90%, 95%, 96%, 97%, 98%, 99% or 100% activity of wild type IL-2 polypeptide. Exemplary IL-2 variants (also referred to as IL-2 muteins) are described herein in the section titled “IL-2 molecule.”

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein. Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a molecule of the disclosure (e.g., polynucleotide, e.g., mRNA). Molecules (e.g., polynucleotides) may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof). In one embodiment, mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Prophylaxis: As used herein, the term “prophylaxis” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

LNPs Comprising IL-2 and/or GM-CSF

Disclosed herein are, inter alia, LNP compositions comprising polynucleotides encoding an IL-2 molecule as well as LNPs comprising polynucleotides encoding GMCSF for use in monotherapy or in combination therapy. In another embodiment, the invention pertains to LNPs comprising: (a) a first polynucleotide encoding an IL-2 molecule; and/or (b) a second polynucleotide encoding a GM-CSF molecule. For example, one LNP can comprise both (a) and (b) or two LNPs (one comprising (a) and one comprising (b)) can be administered. In an embodiment, the first polynucleotide comprises an mRNA encoding an IL-2 molecule, e.g., as described herein. In an embodiment, the second polynucleotide comprises an mRNA encoding a GM-CSF molecule, e.g., as described herein. The LNP compositions of the present disclosure (e.g., comprising a first polynucleotide and/or second polynucleotide) can be used alone or in combination to stimulate regulatory T cells in vivo or ex vivo.

In an aspect, an LNP composition comprising (a) a first polynucleotide encoding an IL-2 molecule; and (b) a second polynucleotide encoding a GM-CSF molecule, comprises: (i) an ionizable lipid, e.g., an amino lipid; (ii) a sterol or other structural lipid; (iii) a non-cationic helper lipid or phospholipid; and (iv) a PEG-lipid.

In an aspect, an LNP composition comprising a polynucleotide encoding an IL-2 molecule comprises: (i) an ionizable lipid, e.g., an amino lipid; (ii) a sterol or other structural lipid; (iii) a non-cationic helper lipid or phospholipid; and (iv) a PEG-lipid.

In an aspect, an LNP composition comprising a polynucleotide encoding a GM-CSF molecule comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating and/or prophylaxis of a disease associated with an aberrant T regulatory cell function in a subject or a method of inhibiting an immune response in a subject. In an embodiment, an LNP composition disclosed herein includes: an LNP comprising a polynucleotide (e.g., a first polynucleotide) encoding an IL-2 molecule, an LNP comprising a polynucleotide (e.g., a second polynucleotide) encoding a GM-CSF molecule; or an LNP comprising both a first polynucleotide encoding an IL-2 molecule and a second polynucleotide encoding a GM-CSF molecule).

In an aspect, an LNP composition comprising a first polynucleotide encoding an IL-2 molecule can be administered alone or in combination with an LNP comprising a second polynucleotide encoding a GM-CSF molecule.

In an aspect, an LNP composition comprising a polynucleotide encoding a GM-CSF molecule can be administered alone or in combination with an LNP comprising a separate polynucleotide encoding an IL-2 molecule.

In an aspect, an LNP composition comprising a first polynucleotide encoding an IL-2 molecule and a second polynucleotide encoding a GM-CSF molecule can be administered alone or in combination with an additional LNP composition, e.g., an LNP composition comprising a third polynucleotide encoding a GM-CSF molecule. In an embodiment, the LNP composition comprising the first polynucleotide encoding the IL-2 molecule and the second polynucleotide encoding the GM-CSF molecule can be administered first, e.g., before administration of the LNP composition comprising the third polynucleotide encoding the GM-CSF molecule. In an embodiment, the order of administration can be reversed, e.g., the LNP composition comprising the first polynucleotide encoding the IL-2 molecule and the second polynucleotide encoding the GM-CSF molecule can be administered after administration of the LNP composition comprising the third polynucleotide encoding the GM-CSF molecule.

Without wishing to be bound by theory, it is believed that, in some embodiments, administration of an LNP comprising GM-CSF alone followed by administration of an LNP comprising IL-2 and GM-CSF, can result in reduced proinflammatory cytokine secretion and reduced Th1 cell activation and/or frequency. Exemplary reduction in Th1 cells with a sequential dosing regimen compared to simultaneous administration is provided in Example 8.

### IL-2 Molecule

Interleukin 2 (IL-2) is a homeostatic cytokine for regulatory T cells (Tregs) which can signal via at least two receptors: the intermediate affinity receptor (dimeric receptor) and the high affinity receptor (trimeric receptor). The intermediate affinity receptor, which consists of Il-2Rβ and the gamma common chain (γc), binds IL-2 with an equilibrium dissociation constant of about 1 nM. The high affinity receptor consists of CD25 (IL-2Rα), IL-2Rβ and the gamma common chain. CD25 is constitutively expressed by regulatory T cells and the high affinity receptor binds IL-2 with an equilibrium dissociation constant of about 10 pM. Thus, regulatory T cells have about a 100-fold greater affinity for IL-2.

Due to the differential affinities of the IL-2 intermediate activity receptor (dimeric) and high affinity receptor (trimeric receptor), and because regulatory T cells constitutively express CD25, there exists about a two-log window in which IL-2 signaling can be activated on regulatory T cells while achieving minimal activation of other IL-2 responsive cells. Without wishing to be bound by theory, it is believed that, in some embodiments, mutations in IL-2 that would confer enhanced differentiation between the high and intermediate IL-2 receptor complexes can be used, e.g., to enhance the regulatory T cell preferential activation. Accordingly, in some embodiments, disclosed herein is an mRNA encoded IL-2 protein that would allow for sustained levels of IL-2 to, e.g., selectively stimulate regulatory T cells. In some embodiments, disclosed herein is a dosing schedule would allow for sustained levels of IL-2 to, e.g., selectively stimulate regulatory T cells.

In an aspect, the disclosure provides an LNP composition comprising a polynucleotide, e.g., a first polynucleotide (e.g., mRNA), encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises a variant of a naturally occurring IL-2 molecule (e.g., an IL-2 variant, e.g., as described herein), or a fragment thereof.

In an embodiment, the LNP composition comprising a polynucleotide encoding an IL-2 molecule can be administered alone or in combination with an LNP composition comprising a polynucleotide encoding a GM-CSF molecule. In an embodiment, the LNP composition comprising the IL-2 molecule and the LNP composition comprising the GM-CSF molecule are administered sequentially. In an embodiment, the LNP composition comprising the IL-2 molecule is administered first and the LNP composition comprising the GM-CSF molecule is administered second. In an embodiment, the LNP composition comprising the IL-2 molecule is administered second and the LNP composition comprising the GM-CSF molecule is administered first. In an embodiment, the LNP composition comprising the IL-2 molecule and the LNP composition comprising the GM-CSF molecule are administered simultaneously, e.g., substantially simultaneously.

In an embodiment, the LNP composition comprising the IL-2 molecule and the LNP composition comprising the GM-CSF molecule are in the same or different compositions.

In an embodiment, the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25). In an embodiment, the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.

In an embodiment, the IL-2 molecule comprises an IL-2 variant, e.g., as described herein. In an embodiment, the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.

In an embodiment, the IL-2 variant comprises a mutation (e.g., substitution) in the IL-2 polypeptide sequence at amino acid 1. In an embodiment, the IL-2 variant comprises a mutation (e.g., substitution) in the IL-2 polypeptide sequence at amino acid 4. In an embodiment, the IL-2 variant comprises a mutation (e.g., substitution) in the IL-2 polypeptide sequence at amino acid 1. In an embodiment, the IL-2 variant comprises a mutation (e.g., substitution) in the IL-2 polypeptide sequence at amino acid 1. In an embodiment, the IL-2 variant comprises a mutation (e.g., substitution) in the IL-2 polypeptide sequence at amino acid 1. In an embodiment, the IL-2 variant comprises a mutation (e.g., substitution) in the IL-2 polypeptide sequence at amino acid 8. In an embodiment, the IL-2 variant comprises a mutation (e.g., substitution) in the IL-2 polypeptide sequence at amino acid 10. In an embodiment, the IL-2 variant comprises a mutation (e.g., substitution) in the IL-2 polypeptide sequence at amino acid 11. In an embodiment, the IL-2 variant comprises a mutation (e.g., substitution) in the IL-2 polypeptide sequence at amino acid 13. In an embodiment, the IL-2 variant comprises a mutation (e.g., substitution) in the IL-2 polypeptide sequence at amino acid 20. In an embodiment, the IL-2 variant comprises a mutation (e.g., substitution) in the IL-2 polypeptide sequence at amino acid 26. In an embodiment, the IL-2 variant comprises a mutation (e.g., substitution) in the IL-2 polypeptide sequence at amino acid 29. In an embodiment, the IL-2 variant comprises a mutation (e.g., substitution) in the IL-2 polypeptide sequence at amino acid 30. In an embodiment, the IL-2 variant comprises a mutation (e.g., substitution) in the IL-2 polypeptide sequence at amino acid 31. In an embodiment, the IL-2 variant comprises a mutation (e.g., substitution) in the IL-2 polypeptide sequence at amino acid 35. In an embodiment, the IL-2 variant comprises a mutation (e.g., substitution) in the IL-2 polypeptide sequence at amino acid 37. In an embodiment, the IL-2 variant comprises a mutation (e.g., substitution) in the IL-2 polypeptide sequence at amino acid 46. In an embodiment, the IL-2 variant comprises a mutation (e.g., substitution) in the IL-2 polypeptide sequence at amino acid 48. In an embodiment, the IL-2 variant comprises a mutation (e.g., substitution) in the IL-2 polypeptide sequence at amino acid 49. In an embodiment, the IL-2 variant comprises a mutation (e.g., substitution) in the IL-2 polypeptide sequence at amino acid 61. In an embodiment, the IL-2 variant comprises a mutation (e.g., substitution) in the IL-2 polypeptide sequence at amino acid 64. In an embodiment, the IL-2 variant comprises a mutation (e.g., substitution) in the IL-2 polypeptide sequence at amino acid 68. In an embodiment, the IL-2 variant comprises a mutation (e.g., substitution) in the IL-2 polypeptide sequence at amino acid 69. In an embodiment, the IL-2 variant comprises a mutation (e.g., substitution) in the IL-2 polypeptide sequence at amino acid 71. In an embodiment, the IL-2 variant comprises a mutation (e.g., substitution) in the IL-2 polypeptide sequence at amino acid 74. In an embodiment, the IL-2 variant comprises a mutation (e.g., substitution) in the IL-2 polypeptide sequence at amino acid 75. In an embodiment, the IL-2 variant comprises a mutation (e.g., substitution) in the IL-2 polypeptide sequence at amino acid 76. In an embodiment, the IL-2 variant comprises a mutation (e.g., substitution) in the IL-2 polypeptide sequence at amino acid 79. In an embodiment, the IL-2 variant comprises a mutation (e.g., substitution) in the IL-2 polypeptide sequence at amino acid 88. In an embodiment, the IL-2 variant comprises a mutation (e.g., substitution) in the IL-2 polypeptide sequence at amino acid 89. In an embodiment, the IL-2 variant comprises a mutation (e.g., substitution) in the IL-2 polypeptide sequence at amino acid 90. In an embodiment, the IL-2 variant comprises a mutation (e.g., substitution) in the IL-2 polypeptide sequence at amino acid 91. In an embodiment, the IL-2 variant comprises a mutation (e.g., substitution) in the IL-2 polypeptide sequence at amino acid 92. In an embodiment, the IL-2 variant comprises a mutation (e.g., substitution) in the IL-2 polypeptide sequence at amino acid 101. In an embodiment, the IL-2 variant comprises a mutation (e.g., substitution) in the IL-2 polypeptide sequence at amino acid 103. In an embodiment, the IL-2 variant comprises a mutation (e.g., substitution) in the IL-2 polypeptide sequence at amino acid 114. In an embodiment, the IL-2 variant comprises a mutation (e.g., substitution) in the IL-2 polypeptide sequence at amino acid 125. In an embodiment, the IL-2 variant comprises a mutation (e.g., substitution) in the IL-2 polypeptide sequence at amino acid 128. In an embodiment, the IL-2 variant comprises a mutation (e.g., substitution) in the IL-2 polypeptide sequence at amino acid 133.

In an embodiment, the IL-2 molecule comprises an IL-2 variant, e.g., as described herein. In an embodiment, the IL-2 variant comprises any one, all or a combination (e.g., 2, 3, 4, 5, or more) of the following mutations (e.g., substitutions): A1T, T3N, T3A, S4P, K8R, T10A, Q11R, L12G, Q13R, L12K, L12Q, L 12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, N26D, N29S, N30S, Y31H, K35R, T37R, M46L, K48E, K49R, E61D, K64R, E68D, V69A, N71T, Q74P, S75P, K76R, H79R, R81A, R81G, R81 S, R81T, D84A, D84E, D84G, D84I, D84M, D84Q D84R, D84S, D84T, S87R, N88A, N88D, N88E, N88L, N88F, N88G, N88M, N88R, N88S, N88V, N88W, I89V, N90H, V91D, V91E, V91G, V91S, V91K, I92T, I92K, I92R, E95G, T101A, F103S, I114V, C125S, Q126L, Q126F, I128T, or T133N. In an embodiment, the IL-2 variant comprises any one, all or a combination (e.g., 2, 3, 4, 5, or more) of the following mutations (e.g., substitutions): A1T, S4P, K8R, T10A, Q1 IR, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.

In an embodiment, the IL-2 variant comprises a A1T mutation. In an embodiment, the IL-2 variant comprises a S4P mutation. In an embodiment, the IL-2 variant comprises a K8R mutation. In an embodiment, the IL-2 variant comprises a T10A mutation. In an embodiment, the IL-2 variant comprises a Q11R mutation. In an embodiment, the IL-2 variant comprises a Q13R mutation. In an embodiment, the IL-2 variant comprises a D20T mutation. In an embodiment, the IL-2 variant comprises a N26D mutation. In an embodiment, the IL-2 variant comprises a N29S mutation. In an embodiment, the IL-2 variant comprises a N30S mutation. In an embodiment, the IL-2 variant comprises a Y31H mutation. In an embodiment, the IL-2 variant comprises a K35R mutation. In an embodiment, the IL-2 variant comprises a T37R mutation. In an embodiment, the IL-2 variant comprises a M46L mutation. In an embodiment, the IL-2 variant comprises a K48E mutation. In an embodiment, the IL-2 variant comprises a K49R mutation. In an embodiment, the IL-2 variant comprises a E61D mutation. In an embodiment, the IL-2 variant comprises a K64R mutation. In an embodiment, the IL-2 variant comprises a E68D mutation. In an embodiment, the IL-2 variant comprises a V69A mutation. In an embodiment, the IL-2 variant comprises a N71T mutation. In an embodiment, the IL-2 variant comprises a Q74P mutation. In an embodiment, the IL-2 variant comprises a S75P mutation. In an embodiment, the IL-2 variant comprises a K76R mutation. In an embodiment, the IL-2 variant comprises a H79R mutation. In an embodiment, the IL-2 variant comprises a N88D mutation. In an embodiment, the IL-2 variant comprises a I89V mutation. In an embodiment, the IL-2 variant comprises a N90H mutation. In an embodiment, the IL-2 variant comprises a V91K mutation. In an embodiment, the IL-2 variant comprises a I92T mutation. In an embodiment, the IL-2 variant comprises a T101A mutation. In an embodiment, the IL-2 variant comprises a F103S mutation. In an embodiment, the IL-2 variant comprises a I114V mutation. In an embodiment, the IL-2 variant comprises a C125S mutation. In an embodiment, the IL-2 variant comprises a I128T mutation. In an embodiment, the IL-2 variant comprises a T133N mutation.

In an embodiment, the IL-2 variant comprises a mutation, e.g., substitution, at position 88 of the IL-2 polypeptide sequence, e.g., an N88D substitution.

In an embodiment, the IL-2 variant comprises a mutation, e.g., substitution, at position 91 of the IL-2 polypeptide sequence, e.g., a V91K substitution.

In an embodiment, the IL-2 variant comprises a mutation, e.g., substitution, at position 91 of the IL-2 polypeptide sequence, e.g., a V69A substitution.

In an embodiment, the IL-2 variant comprises a mutation, e.g., substitution, at position 91 of the IL-2 polypeptide sequence, e.g., a Q74P substitution.

In an embodiment, the IL-2 variant comprises a mutation, e.g., substitution, at position 91 of the IL-2 polypeptide sequence, e.g., a N88D substitution.

In an embodiment, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and/or position 88 of the IL-2 polypeptide sequence, e.g., an N88D substitution.

In an embodiment, the IL-2 variant comprises a mutation, e.g., substitution, at: position 69 of the IL-2 polypeptide sequence, e.g., a V69A substitution; position 74 of the IL-2 polypeptide sequence, e.g., a Q74P substitution; and/or position 91 of the IL-2 polypeptide sequence, e.g., a V91K substitution.

Exemplary IL-2 mutations are described in, Rao et al (2003) Interleukin-2 mutants with enhanced a-receptor subunit binding affinity. Protein Engineering 16(12): pp. 1081-1087; and Rao et al (2005) High-affinity CD25-binding IL-2 mutants potently stimulate persistent T cell growth. Biochemistry 2005(44): pp. 10696-10701, the entire contents of each of which are hereby incorporated by reference in their entireties.

Additional exemplary IL-2 mutations (also referred to as IL-2 muteins) are disclosed in International Application WO 2019/112854, the entire contents of which is hereby incorporated by referenced in its entirety.

In an aspect, an LNP composition disclosed herein comprises a polynucleotide encoding an IL-2 molecule. In an embodiment, the LNP composition comprises a first polynucleotide (e.g., mRNA) encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to an IL-2 amino acid sequence provided in Table 1A or Table 4A. In an embodiment, the IL-2 molecule comprises the amino acid sequence of an IL-2 amino acid sequence provided in Table 1A or Table 4A. In an embodiment, the first polynucleotide (e.g., mRNA) encoding the IL-2 molecule comprises a nucleotide sequence having at least 85%, 90%, 95%, 96%, 97%, 98%, 99% or 100% identity to an IL-2 nucleotide sequence provided in Table 1A or Table 4A. In an embodiment, the first polynucleotide (e.g., mRNA) encoding the IL-2 molecule comprises IL-2 nucleotide sequence provided in Table 1A or Table 4A.

In an aspect, an LNP composition disclosed herein comprises a polynucleotide encoding an IL-2 molecule. In an embodiment, the LNP composition comprises a first polynucleotide (e.g., mRNA) encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to the amino acid sequence of SEQ ID NO: 1 or SEQ ID NO: 30. In an embodiment, the IL-2 molecule comprises the amino acid sequence of SEQ ID NO: 1. In an embodiment, the IL-2 molecule comprising SEQ ID NO: 1, further comprises a leader sequence. In an embodiment, the IL-2 molecule comprises the amino acid sequence of SEQ ID NO: 30. In an embodiment, the first polynucleotide (e.g., mRNA) encoding the IL-2 molecule comprises a nucleotide sequence having at least 85%, 90%, 95%, 96%, 97%, 98%, 99% or 100% identity to the sequence of SEQ ID NO: 7. In an embodiment, the first polynucleotide (e.g., mRNA) encoding the IL-2 molecule comprises the nucleotide sequence of SEQ ID NO: 7.

In an aspect, an LNP composition disclosed herein comprises a polynucleotide encoding an IL-2 molecule. In an embodiment, the LNP composition comprises a first polynucleotide (e.g., mRNA) encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to the amino acid sequence of SEQ ID NO: 11. In an embodiment, the IL-2 molecule comprises the amino acid sequence of SEQ ID NO: 11. In an embodiment, the first polynucleotide (e.g., mRNA) encoding the IL-2 molecule comprises a nucleotide sequence having at least 85%, 90%, 95%, 96%, 97%, 98%, 99% or 100% identity to the sequence of SEQ ID NO: 25. In an embodiment, the first polynucleotide (e.g., mRNA) encoding the IL-2 molecule comprises the nucleotide sequence of SEQ ID NO: 25. In an embodiment, the first polynucleotide (e.g., mRNA) encoding the IL-2 molecule comprises the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In an aspect, an LNP composition disclosed herein comprises a polynucleotide encoding an IL-2 molecule. In an embodiment, the LNP composition comprises a first polynucleotide (e.g., mRNA) encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises an amino acid sequence having at least 85%, 90%, 95%, 96%, 97%, 98%, 99% or 100% identity to the amino acid sequence of SEQ ID NO: 11. In an embodiment, the IL-2 molecule comprises the amino acid sequence of SEQ ID NO: 11. In an embodiment, the first polynucleotide (e.g., mRNA) encoding the IL-2 molecule comprises a nucleotide sequence having at least 85%, 90%, 95%, 96%, 97%, 98%, 99% or 100% identity to the sequence of SEQ ID NO: 36. In an embodiment, the first polynucleotide (e.g., mRNA) encoding the IL-2 molecule comprises the nucleotide sequence of SEQ ID NO: 36. In an embodiment, the first polynucleotide (e.g., mRNA) encoding the IL-2 molecule comprises the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

In an aspect, an LNP composition disclosed herein comprises a polynucleotide encoding an IL-2 molecule. In an embodiment, the LNP composition comprises a first polynucleotide (e.g., mRNA) encoding an IL-2 molecule, e.g., as described herein. In an embodiment, the IL-2 molecule comprises a naturally occurring IL-2 molecule, a fragment of a naturally occurring IL-2 molecule, or a variant thereof. In an embodiment, the IL-2 molecule comprises a variant of a naturally occurring IL-2 molecule (e.g., an IL-2 variant, e.g., as described herein), or a fragment thereof. In an embodiment, the IL-2 molecule (e.g., IL-2 variant) comprises an amino acid sequence having at least 85%, 90%, 95%, 96%, 97%, 98%, 99% or 100% identity to the amino acid sequence of any one of SEQ ID NO: 1, SEQ ID NO: 30, SEQ ID NO: 2, SEQ ID NO: 31, SEQ ID NO: 3, SEQ ID NO: 32, SEQ ID NO: 4, SEQ ID NO: 33, SEQ ID NO: 5, SEQ ID NO: 34, SEQ ID NO: 6 or SEQ ID NO: 35. In an embodiment, the IL-2 molecule (e.g., IL-2 variant) comprises the amino acid sequence of any one of SEQ ID NO: 1, SEQ ID NO: 30, SEQ ID NO: 2, SEQ ID NO: 31, SEQ ID NO: 3, SEQ ID NO: 32, SEQ ID NO: 4, SEQ ID NO: 33, SEQ ID NO: 5, SEQ ID NO: 34, SEQ ID NO: 6 or SEQ ID NO: 35. In an embodiment, the IL-2 molecule (e.g., IL-2 variant) comprising the amino acid sequence of any one of SEQ ID NO: 1, SEQ ID NO: 2, SEQ ID NO: 3, SEQ ID NO: 4, SEQ ID NO: 5 or SEQ ID NO: 6 further comprises a leader sequence. In an embodiment, the first polynucleotide (e.g., mRNA) encoding the IL-2 molecule (e.g., IL-2 variant) comprises a nucleotide sequence having at least 85%, 90%, 95%, 96%, 97%, 98%, 99% or 100% identity to the sequence of SEQ ID NO: 7.

In an aspect, an LNP composition disclosed herein comprises a polynucleotide encoding an IL-2 molecule, e.g., as described herein. In an embodiment, the IL-2 molecule comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the half-life extender is human serum albumin (HSA). In an embodiment, the half-life extender is mouse serum albumin (MSA). In an embodiment, the half-life extender is cyno serum albumin (CSA). In an embodiment, the half-life extender is rat serum albumin (RSA).

In an embodiment, the half-life extender is human serum albumin (HSA). In an embodiment, HSA comprises an amino acid sequence having at least 85%, 90%, 95%, 96%, 97%, 98%, 99% or 100% identity to the amino acid sequence of SEQ ID NO: 8. In an embodiment, HSA comprises the amino acid sequence of SEQ ID NO: 8.

In an embodiment, the LNP comprises a polynucleotide encoding an IL-2 molecule comprising a half-life extender. In an embodiment, the half-life extender is human serum albumin (HSA). In an embodiment, the IL-2 molecule comprising HSA, e.g., HSA-IL-2, comprises an amino acid sequence having at least 85%, 90%, 95%, 96%, 97%, 98%, 99% or 100% identity to an HSA-IL-2 sequence provided in Table 1A. In an embodiment, the IL-2 molecule comprising HSA, e.g., HSA-IL-2, comprises the amino acid sequence of an HSA-IL-2 sequence provided in Table 1A.

In an embodiment, the LNP comprises a polynucleotide encoding an IL-2 molecule comprising a half-life extender. In an embodiment, the half-life extender is human serum albumin (HSA). In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13. In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 without the leader sequence. In an embodiment, the IL-2 molecule comprising HSA, e.g., HSA-IL-2, comprises the amino acid sequence of SEQ ID NO:9, or an amino acid sequence having at least 85%, 90%, 95%, 96%, 97%, 98%, 99% or 100% identity to the amino acid sequence of SEQ ID NO: 9. In an embodiment, the IL-2 molecule comprising HSA, e.g., HSA-IL-2, comprises the amino acid sequence of SEQ ID NO:9 without the leader sequence, or an amino acid sequence having at least 85%, 90%, 95%, 96%, 97%, 98%, 99% or 100% identity to the amino acid sequence of SEQ ID NO: 9 without the leader sequence. In an embodiment, the IL-2 molecule comprising HSA, e.g., HSA-IL-2, comprises the amino acid sequence of SEQ ID NO:10, or an amino acid sequence having at least 85%, 90%, 95%, 96%, 97%, 98%, 99% or 100% identity to the amino acid sequence of SEQ ID NO: 10. In an embodiment, the IL-2 molecule comprising HSA, e.g., HSA-IL-2, comprises the amino acid sequence of SEQ ID NO:10 without the leader sequence, or an amino acid sequence having at least 85%, 90%, 95%, 96%, 97%, 98%, 99% or 100% identity to the amino acid sequence of SEQ ID NO: 10 without the leader sequence. In an embodiment, the IL-2 molecule comprising HSA, e.g., HSA-IL-2, comprises the amino acid sequence of SEQ ID NO:11, or an amino acid sequence having at least 85%, 90%, 95%, 96%, 97%, 98%, 99% or 100% identity to the amino acid sequence of SEQ ID NO: 11. In an embodiment, the IL-2 molecule comprising HSA, e.g., HSA-IL-2, comprises the amino acid sequence of SEQ ID NO:11 without the leader sequence, or an amino acid sequence having at least 85%, 90%, 95%, 96%, 97%, 98%, 99% or 100% identity to the amino acid sequence of SEQ ID NO:11 without the leader sequence. In an embodiment, the IL-2 molecule comprising HSA, e.g., HSA-IL-2, comprises the amino acid sequence of SEQ ID NO:12, or an amino acid sequence having at least 85%, 90%, 95%, 96%, 97%, 98%, 99% or 100% identity to the amino acid sequence of SEQ ID NO: 12. In an embodiment, the IL-2 molecule comprising HSA, e.g., HSA-IL-2, comprises the amino acid sequence of SEQ ID NO:12 without the leader sequence, or an amino acid sequence having at least 85%, 90%, 95%, 96%, 97%, 98%, 99% or 100% identity to the amino acid sequence of SEQ ID NO: 12 without the leader sequence. In an embodiment, the IL-2 molecule comprising HSA, e.g., HSA-IL-2, comprises the amino acid sequence of SEQ ID NO:13, or an amino acid sequence having at least 85%, 90%, 95%, 96%, 97%, 98%, 99% or 100% identity to the amino acid sequence of SEQ ID NO: 13. In an embodiment, the IL-2 molecule comprising HSA, e.g., HSA-IL-2, comprises the amino acid sequence of SEQ ID NO:13 without the leader sequence, or an amino acid sequence having at least 85%, 90%, 95%, 96%, 97%, 98%, 99% or 100% identity to the amino acid sequence of SEQ ID NO: 13 without the leader sequence.

In an embodiment, the LNP comprises a polynucleotide encoding an IL-2 molecule comprising a half-life extender. In an embodiment, the half-life extender is human serum albumin (HSA). In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence of SEQ ID NO:11. In an embodiment, the IL-2 molecule comprising HSA, e.g., HSA-IL-2, comprises the sequence of SEQ ID NO: 11. In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence SEQ ID NO: 11 without the leader sequence. In an embodiment, the IL-2 molecule comprising HSA, e.g., HSA-IL-2, comprises the sequence of SEQ ID NO: 11 without the leader sequence. In an embodiment, the polynucleotide encoding the IL-2 molecule comprises a nucleotide sequence having at least 85%, 90%, 95%, 96%, 97%, 98%, 99% or 100% identity to the sequence of SEQ ID NO: 36. In an embodiment, the polynucleotide (e.g., mRNA) encoding the IL-2 molecule comprises the nucleotide sequence of SEQ ID NO: 36. In an embodiment, the polynucleotide (e.g., mRNA) encoding the IL-2 molecule comprises the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

In an embodiment, the LNP comprises a polynucleotide encoding an IL-2 molecule comprising a half-life extender. In an embodiment, the half-life extender is human serum albumin (HSA). In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence of SEQ ID NO:11. In an embodiment, the IL-2 molecule comprising HSA, e.g., HSA-IL-2, comprises the sequence of SEQ ID NO: 11. In an embodiment, the IL-2 molecule comprising HSA, e.g., HSA-IL-2, comprises an amino acid sequence having at least 85%, 90%, 95%, 96%, 97%, 98%, 99% or 100% identity to the amino acid sequence SEQ ID NO: 11 without the leader sequence. In an embodiment, the IL-2 molecule comprising HSA, e.g., HSA-IL-2, comprises the sequence of SEQ ID NO: 11 without the leader sequence. In an embodiment, the polynucleotide encoding the IL-2 molecule comprises a nucleotide sequence having at least 85%, 90%, 95%, 96%, 97%, 98%, 99% or 100% identity to the sequence of SEQ ID NO: 25. In an embodiment, the polynucleotide (e.g., mRNA) encoding the IL-2 molecule comprises the nucleotide sequence of SEQ ID NO: 25. In an embodiment, the polynucleotide (e.g., mRNA) encoding the IL-2 molecule comprises the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In an embodiment, the polynucleotide (e.g., mRNA) encoding the IL-2 molecule further comprises one or more elements, e.g., a 5′ UTR and/or a 3′ UTR disclosed herein, e.g., in Table 4A. In an embodiment, the 5′ UTR and/or 3′UTR comprise one or more micro RNA (mIR) binding sites, e.g., as disclosed herein. Exemplary 5′ UTRs and 3′ UTRs are disclosed in the section entitled “5′ UTR and 3′UTR” herein.

TABLE 1A
Exemplary IL-2 sequences, human serum albumin (HSA) sequences and HSA-
IL-2 sequences

| SEQ | | |
|---|---|---|
| ID | Sequence | |
| NO | information | Sequence |
| 1 | Human IL-2 | APTSSSTKKTQLQLEHLLLDLQMILNGINNYKNPKLTRMLTFKF |
| | | YMPKKATELKHLQCLEEELKPLEEVLNLAQSKNFHLRPRDLIS |
| | | NINVIVLELKGSETTFMCEYADETATIVEFLNRWITFCQSIISTLT |
| 30 | Human IL-2 | MYRMQLLSCIALSLALVTNSAPTSSSTKKTQLQLEHLLLDLQMI |
| | | LNGINNYKNPKLTRMLTFKFYMPKKATELKHLQCLEEELKPLE |
| | | EVLNLAQSKNFHLRPRDLISNINVIVLELKGSETTFMCEYADET |
| | | ATIVEFLNRWITFCQSIISTLT |
| 2 | Human IL-2 | APTSSSTKKTQLQLEHLLLDLQMILNGINNYKNPKLTRMLTFKF |
| | N88D | YMPKKATELKHLQCLEEELKPLEEVLNLAQSKNFHLRPRDLIS |
| | | DINVIVLELKGSETTFMCEYADETATIVEFLNRWITFCQSIISTLT |
| 31 | Human IL-2 | MYRMQLLSCIALSLALVTNSAPTSSSTKKTQLQLEHLLLDLQMI |
| | N88D | LNGINNYKNPKLTRMLTFKFYMPKKATELKHLQCLEEELKPLE |
| | | EVLNLAQSKNFHLRPRDLISDINVIVLELKGSETTFMCEYADET |
| | | ATIVEFLNRWITFCQSIISTLT |
| 3 | Human IL-2 | APTSSSTKKTQLQLEHLLLDLQMILNGINNYKNPKLTRMLTFKF |
| | V69A, Q74P, | YMPKKATELKHLQCLEEELKPLEEALNLAPSKNFHLRPRDLIS |
| | N88D | DINVIVLELKGSETTFMCEYADETATIVEFLNRWITFCQSIISTLT |
| 32 | Human IL-2 | MYRMQLLSCIALSLALVTNSAPTSSSTKKTQLQLEHLLLDLQMI |
| | V69A, Q74P, | LNGINNYKNPKLTRMLTFKFYMPKKATELKHLQCLEEELKPLE |
| | N88D | EALNLAPSKNFHLRPRDLISDINVIVLELKGSETTFMCEYADET |
| | | ATIVEFLNRWITFCQSIISTLT |
| 4 | Human IL-2, | APTSSSTKKTQLQLEHLLLDLQMILNGINNYKNPKLTRMLTFKF |
| | V91K | YMPKKATELKHLQCLEEELKPLEEVLNLAQSKNFHLRPRDLIS |
| | | NINKIVLELKGSETTFMCEYADETATIVEFLNRWITFCQSIISTLT |
| 33 | Human IL-2, | MYRMQLLSCIALSLALVTNSAPTSSSTKKTQLQLEHLLLDLQMI |
| | V91K | LNGINNYKNPKLTRMLTFKFYMPKKATELKHLQCLEEELKPLE |
| | | EVLNLAQSKNFHLRPRDLISNINKIVLELKGSETTFMCEYADET |
| | | ATIVEFLNRWITFCQSIISTLT |
| 5 | Human IL-2 | APTSSSTKKTQLQLEHLLLDLQMILNGINNYKNPKLTRMLTFKF |
| | V69A, Q74P, | YMPKKATELKHLQCLEEELKPLEEALNLAPSKNFHLRPRDLIS |
| | V91K | NINKIVLELKGSETTFMCEYADETATIVEFLNRWITFCQSIISTLT |
| 34 | Human IL-2 | MYRMQLLSCIALSLALVTNSAPTSSSTKKTQLQLEHLLLDLQMI |
| | V69A, Q74P, | LNGINNYKNPKLTRMLTFKFYMPKKATELKHLQCLEEELKPLE |
| | V91K | EALNLAPSKNFHLRPRDLISNINKIVLELKGSETTFMCEYADET |
| | | ATIVEFLNRWITFCQSIISTLT |
| 6 | Human IL-2 | APTSSSTKKTQLQLEHLLLTLQMILNGINNYKNPKLTRMLTFKF |
| | D20T | YMPKKATELKHLQCLEEELKPLEEVLNLAQSKNFHLRPRDLIS |
| | | NINVIVLELKGSETTFMCEYADETATIVEFLNRWITFCQSIISTLT |
| 35 | Human IL-2 | MYRMQLLSCIALSLALVTNSAPTSSSTKKTQLQLEHLLLTLQMI |
| | D20T | LNGINNYKNPKLTRMLTFKFYMPKKATELKHLQCLEEELKPLE |
| | | EVLNLAQSKNFHLRPRDLISNINVIVLELKGSETTFMCEYADET |
| | | ATIVEFLNRWITFCQSIISTLT |
| 7 | Human IL-2 | AGUUCCCUAU CACUCUCUUU AAUCACUACU |
| | mRNA | CACAGUAACC UCAACUCCUG CCACAAUGUA |
| | sequence | CAGGAUGCAA CUCCUGUCUU GCAUUGCACU |
| | | AAGUCUUGCA CUUGUCACAA ACAGUGCACC |
| | | UACUUCAAGU UCUACAAAGA AAACACAGCU |
| | | ACAACUGGAG CAUUUACUGC UGGAUUUACA |
| | | GAUGAUUUUG AAUGGAAUUA AUAAUUACAA |
| | | GAAUCCCAAA CUCACCAGGA UGCUCACAUU |
| | | UAAGUUUUAC AUGCCCAAGA AGGCCACAGA |
| | | ACUGAAACAU CUUCAGUGUC UAGAAGAAGA |
| | | ACUCAAACCU CUGGAGGAAG UGCUAAAUUU |
| | | AGCUCAAAGC AAAAACUUUC ACUUAAGACC |
| | | CAGGGACUUA AUCAGCAAUA UCAACGUAAU |
| | | AGUUCUGGAA CUAAAGGGAU CUGAAACAAC |
| | | AUUCAUGUGU GAAUAUGCUG AUGAGACAGC |
| | | AACCAUUGUA GAAUUUCUGA ACAGAUGGAU |
| | | UACCUUUUGU CAAAGCAUCA UCUCAACACU |
| | | GACUUGAUAA UUAAGUGCUU CCCACUUAAA |
| | | ACAUAUCAGG CCUUCUAUUU AUUUAAAUAU |
| | | UUAAAUUUUA UAUUUAUUGU UGAAUGUAUG |
| | | GUUUGCUACC UAUUGUAACU AUUAUUCUUA |
| | | AUCUUAAAAC UAUAAAUAUG GAUCUUUUAU |
| | | GAUUCUUUUU GUAAGCCCUA GGGGCUCUAA |
| | | AAUGGUUUCA CUUAUUUAUC CCAAAAUAUU |
| | | UAUUAUUAUG UUGAAUGUUA AAUAUAGUAU |
| | | CUAUGUAGAU UGGUUAGUAA AACUAUUUAA |
| | | UAAAUUUGAU AAAUAUAAAA AAAAAAAAAA |
| | | AAAAAAAAAA AA |
| 8 | Human | AHKSEVAHRFKDLGEENFKALVLIAFAQYLQQCPFEDHVKLVN |
| | serum | EVTEFAKTCVADESAENCDKSLHTLFGDKLCTVATLRETYGE |
| | albumin | MADCCAKQEPERNECFLQHKDDNPNLPRLVRPEVDVMCTAFH |
| | (HSA) | DNEETFLKKYLYEIARRHPYFYAPELLFFAKRYKAAFTECCQA |
| | | ADKAACLLPKLDELRDEGKASSAKQRLKCASLQKFGERAFKA |
| | | WAVARLSQRFPKAEFAEVSKLVTDLTKVHTECCHGDLLECAD |
| | | DRADLAKYICENQDSISSKLKECCEKPLLEKSHCIAEVENDEMP |
| | | ADLPSLAADFVESKDVCKNYAEAKDVFLGMFLYEYARRHPDY |
| | | SVVLLLRLAKTYETTLEKCCAAADPHECYAKVFDEFKPLVEEP |
| | | QNLIKQNCELFEQLGEYKFQNALLVRYTKKVPQVSTPTLVEVS |
| | | RNLGKVGSKCCKHPEAKRMPCAEDYLSVVLNQLCVLHEKTPV |
| | | SDRVTKCCTESLVNRRPCFSALEVDETYVPKEFNAETFTFHADI |
| | | CTLSEKERQIKKQTALVELVKHKPKATKEQLKAVMDDFAAFV |
| | | EKCCKADDKETCFAEEGKKLVAASQAALGL |
| 9 | Leader- | MKWVTFISLLFLFSSAYSRGVFRRDAHKSEVAHRFKDLGEEN |
| | HSA-(G3S)- | FKALVLIAFAQYLQQCPFEDHVKLVNEVTEFAKTCVADESAEN |
| | (Human | CDKSLHTLFGDKLCTVATLRETYGEMADCCAKQEPERNECFL |
| | IL-2) | QHKDDNPNLPRLVRPEVDVMCTAFHDNEETFLKKYLYEIARR |
| | | HPYFYAPELLFFAKRYKAAFTECCQAADKAACLLPKLDELRDE |
| | | GKASSAKQRLKCASLQKFGERAFKAWAVARLSQRFPKAEFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVLLLRLAKTYETTLE |
| | | KCCAAADPHECYAKVFDEFKPLVEEPQNLIKQNCELFEQLGEY |
| | | KFQNALLVRYTKKVPQVSTPTLVEVSRNLGKVGSKCCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLTRMLTFKFYMPKKATELKHLQCLEEELKPLE |
| | | EVLNLAQSKNFHLRPRDLISNINVIVLELKGSETTFMCEYADET |
| | | ATIVEFLNRWITFCQSIISTLT |
| 10 | Leader- | MKWVTFISLLFLFSSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2 N88D) | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLVRYTKKVPQVSTPTLVEVSRNLGKVGSKCCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEEELKP |
| | | LEEVLNLAQSKNFHLRPRDLISDINVIVLELKGSETTFMCEYAD |
| | | ETATIVEFLNRWITFCQSIISTLT |
| 11 | Leader- | MKWVTFISLLFLFSSAYSRGVFRRDAHKSEVAHRFKDLGEEN |
| | HSA-(G3S)- | FKALVLIAFAQYLQQCPFEDHVKLVNEVTEFAKTCVADESAEN |
| | (Human | CDKSLHTLFGDKLCTVATLRETYGEMADCCAKQEPERNECFL |
| | IL-2 V69A, | QHKDDNPNLPRLVRPEVDVMCTAFHDNEETFLKKYLYEIARR |
| | Q74P, | HPYFYAPELLFFAKRYKAAFTECCQAADKAACLLPKLDELRDE |
| | N88D) | GKASSAKQRLKCASLQKFGERAFKAWAVARLSQRFPKAEFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVLLLRLAKTYETTLE |
| | | KCCAAADPHECYAKVFDEFKPLVEEPQNLIKQNCELFEQLGEY |
| | | KFQNALLVRYTKKVPQVSTPTLVEVSRNLGKVGSKCCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLTRMLTFKFYMPKKATELKHLQCLEEELKPLE |
| | | EALNLAPSKNFHLRPRDLISDINVIVLELKGSETTFMCEYADET |
| | | ATIVEFLNRWITFCQSIISTLT |
| 36 | Leader- | AUGAAGUGGGUGACCUUCAUCAGCCUGCUGUUCCUGUUCA |
| | HSA-(G3S)- | GCAGCGCCUACAGCAGAGGCGUGUUCAGAAGAGACGCCCA |
| | (Human | CAAGAGCGAGGUGGCCCACAGAUUCAAGGACCUGGGCGAG |
| | IL-2 V69A, | GAGAACUUCAAGGCCCUGGUGCUGAUCGCCUUCGCCCAGU |
| | Q74P, | ACCUGCAGCAGUGCCCUUUCGAGGACCACGUGAAGCUGGU |
| | N88D) ORF | GAACGAGGUGACCGAGUUCGCCAAGACCUGCGUGGCCGAC |
| | mRNA | GAGAGCGCCGAGAACUGCGACAAGAGCCUGCACACCCUGU |
| | sequence | UCGGCGACAAGCUGUGCACCGUGGCCACCCUGAGAGAAAC |
| | | UUACGGCGAGAUGGCCGACUGCUGCGCCAAGCAGGAGCCA |
| | | GAGCGGAACGAGUGCUUCCUCCAGCACAAGGACGACAACC |
| | | CUAACCUGCCUAGACUGGUAAGGCCUGAGGUGGACGUGAU |
| | | GUGUACCGCCUUCCACGACAACGAGGAGACAUUCCUGAAG |
| | | AAGUACCUGUACGAGAUCGCCAGAAGACACCCUUACUUCU |
| | | ACGCCCCUGAGUUGCUGUUCUUCGCGAAGAGAUACAAGGC |
| | | CGCCUUCACCGAGUGCUGCCAGGCCGCCGAUAAGGCCGCG |
| | | UGCCUGCUGCCUAAGCUGGACGAGCUGAGAGACGAGGGCA |
| | | AGGCAUCCAGCGCUAAGCAGAGACUGAAGUGCGCCAGCCU |
| | | GCAGAAGUUCGGAGAGAGAGCUUUCAAGGCGUGGGCAGU |
| | | GGCUAGAUUGAGCCAAAGAUUCCCUAAGGCAGAAUUCGCU |
| | | GAGGUGAGCAAGCUCGUGACUGACCUGACCAAGGUGCAUA |
| | | CAGAGUGCUGUCACGGCGACCUGCUGGAGUGCGCCGACGA |
| | | CAGAGCCGACCUGGCCAAGUACAUCUGCGAGAACCAGGAC |
| | | AGCAUCAGCAGCAAGCUGAAGGAGUGUUGUGAGAAGCCU |
| | | CUAUUGGAGAAGAGUCACUGCAUUGCCGAGGUGGAGAAC |
| | | GACGAGAUGCCUGCGGAUCUGCCAAGCUUGGCGGCCGACU |
| | | UCGUGGAGAGCAAGGACGUGUGCAAGAACUACGCCGAGGC |
| | | CAAGGACGUUUUCCUGGGCAUGUUCCUCUACGAGUACGCA |
| | | CGCAGACAUCCAGACUACAGCGUGGUGCUGCUGCUGAGAC |
| | | UGGCUAAGACAUACGAAACUACCCUGGAGAAGUGCUGCGC |
| | | AGCGGCGGACCCUCACGAGUGUUACGCCAAGGUGUUCGAC |
| | | GAGUUCAAGCCUCUGGUGGAGGAGCCUCAGAACCUGAUCA |
| | | AGCAGAACUGUGAGCUGUUCGAGCAGCUCGGCGAGUACAA |
| | | GUUCCAGAACGCCCUGUUGGUCCGCUACACCAAGAAGGUG |
| | | CCUCAAGUAAGUACCCCUACCCUGGUAGAGGUUAGUAGAA |
| | | ACCUGGGCAAGGUGGGCAGCAAGUGCUGUAAGCACCCAGA |
| | | AGCUAAGAGGAUGCCUUGCGCCGAGGACUACCUGUCCGUU |
| | | GUGCUGAACCAGCUGUGCGUGCUGCACGAGAAGACCCCUG |
| | | UGAGCGACAGAGUGACAAAGUGUUGCACCGAGAGCUUAG |
| | | UGAAUAGAAGACCUUGCUUCAGCGCCCUGGAAGUUGACGA |
| | | AACUUACGUGCCUAAGGAGUUCAACGCCGAGACUUUCACA |
| | | UUCCACGCCGACAUUUGCACUCUGAGCGAGAAGGAGAGAC |
| | | AGAUCAAGAAGCAGACCGCCCUCGUAGAGUUGGUCAAGCA |
| | | CAAGCCGAAGGCAACCAAGGAACAGCUUAAGGCCGUGAUG |
| | | GACGACUUCGCAGCCUUCGUCGAGAAGUGUUGUAAGGCCG |
| | | ACGACAAGGAGACUUGCUUCGCAGAGGAAGGCAAGAAGU |
| | | UGGUAGCCGCCUCUCAGGCUGCCUUGGGACUCGGUGGCGG |
| | | CAGCGCCCCUACCAGCAGCUCCACAAAGAAGACGCAGCUG |
| | | CAGCUGGAGCACCUGCUACUAGACUUACAGAUGAUCCUGA |
| | | ACGGCAUCAACAACUACAAGAACCCGAAGCUCACCAGAAU |
| | | GCUGACAUUCAAGUUCUACAUGCCUAAGAAGGCCACUGAG |
| | | CUGAAGCACCUUCAGUGCCUGGAGGAGGAACUUAAGCCAC |
| | | UGGAAGAGGCCCUGAACCUGGCCCCUAGUAAGAAUUUCCA |
| | | CCUCAGACCUCGCGACCUUAUUAGCGACAUCAACGUGAUC |
| | | GUGCUCGAAUUAAAGGGCUCCGAGACAACAUUCAUGUGCG |
| | | AAUACGCCGACGAGACAGCCACCAUCGUGGAGUUCCUGAA |
| | | CCGCUGGAUCACCUUCUGCCAGAGCAUCAUCUCUACUCUG |
| | | ACC |
| 12 | Leader- | MKWVTFISLLFLFSSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2, V91K) | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLVRYTKKVPQVSTPTLVEVSRNLGKVGSKCCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEEELKP |
| | | LEEVLNLAQSKNFHLRPRDLISNINKIVLELKGSETTFMCEYAD |
| | | ETATIVEFLNRWITFCQSIISTLT |
| 13 | Leader- | MKWVTFISLLFLFSSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2 V69A, | LQHKDDNPNLPRLVRPEVDVMCTAFHDNEETFLKKYLYEIAR |
| | Q74P, | RHPYFYAPELLFFAKRYKAAFTECCQAADKAACLLPKLDELR |
| | V91K | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLVRYTKKVPQVSTPTLVEVSRNLGKVGSKCCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEEELKP |
| | | LEEALNLAPSKNFHLRPRDLISNINKIVLELKGSETTFMCEYAD |
| | | ETATIVEFLNRWITFCQSIISTLT |

Without wishing to be bound by theory, a skilled person would understand that in some embodiments the amino acid sequence of RGVFRRD can constitute part of the leader sequence described herein as HSA is generally made as a pre-pro-peptide.

In some embodiments, a polynucleotide of the present disclosure, for example a polynucleotide comprising an mRNA nucleotide sequence encoding a polypeptide, comprises (1) a 5′ cap, e.g., as disclosed herein, (2) a 5′ UTR, e.g., as provided in Table 4A, (3) a nucleotide sequence ORF provided in Table 1A, or 4A, e.g., chosen from: SEQ ID NO: 25, SEQ ID NO: 7 or SEQ ID NO: 36, (4) a stop codon, (5) a 3′UTR, e.g., as provided in Table 4A, and (6) a poly-A tail, e.g., as disclosed herein, e.g., a poly-A tail of about 100 residues, e.g., SEQ ID NO: 29.

In some embodiments, a polynucleotide comprising an mRNA nucleotide sequence encoding an IL-2 polypeptide, comprises SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

In some embodiments, a polynucleotide comprising an mRNA nucleotide sequence encoding an IL-2 polypeptide, comprises SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

TABLE 4A
Exemplary IL-2 construct sequences
Note: “G5″ indicat”s that all uracils (U) in the mRNA are replaced by N1-methylpseudouracils.

| | mRNA | ORF Sequence | ORF Sequence | 5′ UTR | 3′ UTR | Construct |
|---|---|---|---|---|---|---|
| | Name | (Amino Acid) | (Nucleotide) | Sequence | Sequence | Sequence |
| SEQ | | | | | | |
| ID | | | | | | |
| NO: | | 11 | 25 | 26 | 27 | 28 |
| | HSA- | MKWVTFISLLFLFSSAYS | AUGAAGUGGGUGACC | GGGAAA | UGAUAA | SEQ ID |
| | hsIL2.V69A. | RGVFRRDAHKSEVAHR | UUCAUCAGCCUGCUG | UAAGAG | UAGGCU | NO: 28 |
| | Q74P.N88D | FKDLGEENFKALVLIAFA | UUCCUGUUCAGCAGC | AGAAAA | GGAGCC | consists |
| | G5 | QYLQQCPFEDHVKLVN | GCCUACAGCAGAGGC | GAAGAG | UCGGUG | from 5′ to |
| | Cap: C1 | EVTEFAKTCVADESAEN | GUGUUCAGAAGAGAC | UAAGAA | GCCUAGC | 3′ end: 5′ |
| | Poly A | CDKSLHTLFGDKLCTVA | GCCCACAAGAGCGAG | GAAAUA | UUCUUG | UTR of |
| | tail: 100 nt | TLRETYGEMADCCAKQ | GUGGCCCACAGAUUC | UAAGACC | CCCCUUG | SEQ ID |
| | (SEQ ID NO: | EPERNECFLQHKDDNP | AAGGACCUGGGCGAG | CCGGCGC | GGCCUCC | NO: 26, |
| | 29) | NLPRLVRPEVDVMCTA | GAGAACUUCAAGGCC | CGCCACC | CCCCAGC | ORF |
| | | FHDNEETFLKKYLYEIAR | CUGGUGCUGAUCGCC | | CCCUCCU | sequence of |
| | | RHPYFYAPELLFFAKRYK | UUCGCCCAGUACCUG | | CCCCUUC | SEQ ID NO: |
| | | AAFTECCQAADKAACL | CAGCAGUGCCCUUUC | | CUGCACC | 25, 3′ |
| | | LPKLDELRDEGKASSAK | GAGGACCACGUGAAG | | CGUACCC | UTR of |
| | | QRLKCASLQKFGERAFK | CUGGUGAACGAGGUG | | CCGUGG | SEQ ID |
| | | AWAVARLSQRFPKAEF | ACCGAGUUCGCCAAG | | UCUUUG | NO: 27 |
| | | AEVSKLVTDLTKVHTEC | ACCUGCGUGGCCGAC | | AAUAAA | and Poly |
| | | CHGDLLECADDRADLA | GAGAGCGCCGAGAAC | | GUCUGA | A tail of |
| | | KYICENQDSISSKLKECC | UGCGACAAGAGCCUG | | GUGGGC | SEQ ID |
| | | EKPLLEKSHCIAEVENDE | CACACCCUGUUCGGC | | GGC | NO: 29 |
| | | MPADLPSLAADFVESK | GACAAGCUGUGCACC | | | |
| | | DVCKNYAEAKDVFLGM | GUGGCCACCCUGAGA | | | |
| | | FLYEYARRHPDYSVVLLL | GAAACUUACGGCGAG | | | |
| | | RLAKTYETTLEKCCAAA | AUGGCCGACUGCUGC | | | |
| | | DPHECYAKVFDEFKPLV | GCCAAGCAGGAGCCA | | | |
| | | EEPQNLIKQNCELFEQL | GAGCGGAACGAGUGC | | | |
| | | GEYKFQNALLVRYTKKV | UUCCUCCAGCACAAG | | | |
| | | PQVSTPTLVEVSRNLGK | GACGACAACCCUAACC | | | |
| | | VGSKCCKHPEAKRMPC | UGCCUAGACUGGUAA | | | |
| | | AEDYLSVVLNQLCVLHE | GGCCUGAGGUGGACG | | | |
| | | KTPVSDRVTKCCTESLV | UGAUGUGUACCGCCU | | | |
| | | NRRPCFSALEVDETYVP | UCCACGACAACGAGG | | | |
| | | KEFNAETFTFHADICTLS | AGACAUUCCUGAAGA | | | |
| | | EKERQIKKQTALVELVK | AGUACCUGUACGAGA | | | |
| | | HKPKATKEQLKAVMDD | UCGCCAGAAGACACC | | | |
| | | FAAFVEKCCKADDKETC | CUUACUUCUACGCCC | | | |
| | | FAEEGKKLVAASQAAL | CUGAGUUGCUGUUC | | | |
| | | GLGGGSAPTSSSTKKTQ | UUCGCGAAGAGAUAC | | | |
| | | LQLEHLLLDLQMILNGI | AAGGCCGCCUUCACC | | | |
| | | NNYKNPKLTRMLTFKFY | GAGUGCUGCCAGGCC | | | |
| | | MPKKATELKHLQCLEEE | GCCGAUAAGGCCGCG | | | |
| | | LKPLEEALNLAPSKNFHL | UGCCUGCUGCCUAAG | | | |
| | | RPRDLISDINVIVLELKG | CUGGACGAGCUGAGA | | | |
| | | SETTFMCEYADETATIV | GACGAGGGCAAGGCA | | | |
| | | EFLNRWITFCQSIISTLT | UCCAGCGCUAAGCAG | | | |
| | | | AGACUGAAGUGCGCC | | | |
| | | | AGCCUGCAGAAGUUC | | | |
| | | | GGAGAGAGAGCUUUC | | | |
| | | | AAGGCGUGGGCAGUG | | | |
| | | | GCUAGAUUGAGCCAA | | | |
| | | | AGAUUCCCUAAGGCA | | | |
| | | | GAAUUCGCUGAGGUG | | | |
| | | | AGCAAGCUCGUGACU | | | |
| | | | GACCUGACCAAGGUG | | | |
| | | | CAUACAGAGUGCUGU | | | |
| | | | CACGGCGACCUGCUG | | | |
| | | | GAGUGCGCCGACGAC | | | |
| | | | AGAGCCGACCUGGCC | | | |
| | | | AAGUACAUCUGCGAG | | | |
| | | | AACCAGGACAGCAUC | | | |
| | | | AGCAGCAAGCUGAAG | | | |
| | | | GAGUGUUGUGAGAA | | | |
| | | | GCCUCUAUUGGAGAA | | | |
| | | | GAGUCACUGCAUUGC | | | |
| | | | CGAGGUGGAGAACGA | | | |
| | | | CGAGAUGCCUGCGGA | | | |
| | | | UCUGCCAAGCUUGGC | | | |
| | | | GGCCGACUUCGUGGA | | | |
| | | | GAGCAAGGACGUGUG | | | |
| | | | CAAGAACUACGCCGA | | | |
| | | | GGCCAAGGACGUUUU | | | |
| | | | CCUGGGCAUGUUCCU | | | |
| | | | CUACGAGUACGCACG | | | |
| | | | CAGACAUCCAGACUA | | | |
| | | | CAGCGUGGUGCUGCU | | | |
| | | | GCUGAGACUGGCUAA | | | |
| | | | GACAUACGAAACUAC | | | |
| | | | CCUGGAGAAGUGCUG | | | |
| | | | CGCAGCGGCGGACCC | | | |
| | | | UCACGAGUGUUACGC | | | |
| | | | CAAGGUGUUCGACGA | | | |
| | | | GUUCAAGCCUCUGGU | | | |
| | | | GGAGGAGCCUCAGAA | | | |
| | | | CCUGAUCAAGCAGAA | | | |
| | | | CUGUGAGCUGUUCGA | | | |
| | | | GCAGCUCGGCGAGUA | | | |
| | | | CAAGUUCCAGAACGC | | | |
| | | | CCUGUUGGUCCGCUA | | | |
| | | | CACCAAGAAGGUGCC | | | |
| | | | UCAAGUAAGUACCCC | | | |
| | | | UACCCUGGUAGAGGU | | | |
| | | | UAGUAGAAACCUGGG | | | |
| | | | CAAGGUGGGCAGCAA | | | |
| | | | GUGCUGUAAGCACCC | | | |
| | | | AGAAGCUAAGAGGAU | | | |
| | | | GCCUUGCGCCGAGGA | | | |
| | | | CUACCUGUCCGUUGU | | | |
| | | | GCUGAACCAGCUGUG | | | |
| | | | CGUGCUGCACGAGAA | | | |
| | | | GACCCCUGUGAGCGA | | | |
| | | | CAGAGUGACAAAGUG | | | |
| | | | UUGCACCGAGAGCUU | | | |
| | | | AGUGAAUAGAAGACC | | | |
| | | | UUGCUUCAGCGCCCU | | | |
| | | | GGAAGUUGACGAAAC | | | |
| | | | UUACGUGCCUAAGGA | | | |
| | | | GUUCAACGCCGAGAC | | | |
| | | | UUUCACAUUCCACGC | | | |
| | | | CGACAUCUGUACUCU | | | |
| | | | GAGCGAGAAGGAGAG | | | |
| | | | ACAGAUCAAGAAGCA | | | |
| | | | GACCGCCCUCGUAGA | | | |
| | | | GUUGGUCAAGCACAA | | | |
| | | | GCCGAAGGCAACCAA | | | |
| | | | GGAACAGCUUAAGGC | | | |
| | | | CGUGAUGGACGACUU | | | |
| | | | CGCAGCCUUCGUCGA | | | |
| | | | GAAGUGUUGUAAGG | | | |
| | | | CCGACGACAAGGAGA | | | |
| | | | CUUGCUUCGCAGAGG | | | |
| | | | AAGGCAAGAAGUUGG | | | |
| | | | UAGCCGCCUCUCAGG | | | |
| | | | CUGCCUUGGGACUCG | | | |
| | | | GUGGCGGCAGCGCCC | | | |
| | | | CUACCAGCAGCUCCAC | | | |
| | | | AAAGAAGACGCAGCU | | | |
| | | | GCAGCUGGAGCACCU | | | |
| | | | GCUACUAGACUUACA | | | |
| | | | GAUGAUCCUGAACGG | | | |
| | | | CAUCAACAACUACAA | | | |
| | | | GAACCCGAAGCUCAC | | | |
| | | | CAGAAUGCUGACAUU | | | |
| | | | CAAGUUCUACAUGCC | | | |
| | | | UAAGAAGGCCACUGA | | | |
| | | | GCUGAAGCACCUUCA | | | |
| | | | GUGCCUGGAGGAGGA | | | |
| | | | ACUUAAGCCACUGGA | | | |
| | | | AGAGGCCCUGAACCU | | | |
| | | | GGCCCCUAGUAAGAA | | | |
| | | | UUUCCACCUCAGACC | | | |
| | | | UCGCGACCUUAUUAG | | | |
| | | | CGACAUCAACGUGAU | | | |
| | | | CGUGCUCGAAUUAAA | | | |
| | | | GGGCUCCGAGACAAC | | | |
| | | | AUUCAUGUGCGAAUA | | | |
| | | | CGCCGACGAGACAGC | | | |
| | | | CACCAUCGUGGAGUU | | | |
| | | | CCUGAACCGCUGGAU | | | |
| | | | CACCUUCUGCCAGAG | | | |
| | | | CAUCAUCUCUACUCU | | | |
| | | | GACC | | | |
| SEQ | | | | | | |
| ID | | | | | | |
| NO: | | 11 | 36 | 26 | 27 | 37 |
| | HSA- | MKWVTFISLLFLFSSAYS | AUGAAGUGGGUGA | GGGAAA | UGAUAA | SEQ ID |
| | hsIL2.V69A. | RGVFRRDAHKSEVAHR | CCUUCAUCAGCCUG | UAAGAG | UAGGCU | NO: 37 |
| | Q74P.N88D | FKDLGEENFKALVLIAFA | CUGUUCCUGUUCA | AGAAAA | GGAGCC | consists |
| | G5 | QYLQQCPFEDHVKLVN | GCAGCGCCUACAGC | GAAGAG | UCGGUG | from 5′ to |
| | Cap: C1 | EVTEFAKTCVADESAEN | AGAGGCGUGUUCA | UAAGAA | GCCUAGC | 3′ end: 5′ |
| | Poly A | CDKSLHTLFGDKLCTVA | GAAGAGACGCCCAC | GAAAUA | UUCUUG | UTR of |
| | tail: 100 nt | TLRETYGEMADCCAKQ | AAGAGCGAGGUGG | UAAGACC | CCCCUUG | SEQ ID |
| | (SEQ ID NO: | EPERNECFLQHKDDNP | CCCACAGAUUCAAG | CCGGCGC | GGCCUCC | NO: 26, |
| | 29) | NLPRLVRPEVDVMCTA | GACCUGGGCGAGGA | CGCCACC | CCCCAGC | ORF |
| | | FHDNEETFLKKYLYEIAR | GAACUUCAAGGCCC | | CCCUCCU | sequence |
| | | RHPYFYAPELLFFAKRYK | UGGUGCUGAUCGC | | CCCCUUC | of SEQ |
| | | AAFTECCQAADKAACL | CUUCGCCCAGUACC | | CUGCACC | ID NO: |
| | | LPKLDELRDEGKASSAK | UGCAGCAGUGCCCU | | CGUACCC | 36, 3′ |
| | | QRLKCASLQKFGERAFK | UUCGAGGACCACGU | | CCGUGG | UTR of |
| | | AWAVARLSQRFPKAEF | GAAGCUGGUGAAC | | UCUUUG | SEQ ID |
| | | AEVSKLVTDLTKVHTEC | GAGGUGACCGAGU | | AAUAAA | NO: 27 |
| | | CHGDLLECADDRADLA | UCGCCAAGACCUGC | | GUCUGA | and Poly |
| | | KYICENQDSISSKLKECC | GUGGCCGACGAGAG | | GUGGGC | A tail of |
| | | EKPLLEKSHCIAEVENDE | CGCCGAGAACUGCG | | GGC | SEQ ID |
| | | MPADLPSLAADFVESK | ACAAGAGCCUGCAC | | | NO: 29 |
| | | DVCKNYAEAKDVFLGM | ACCCUGUUCGGCGA | | | |
| | | FLYEYARRHPDYSVVLLL | CAAGCUGUGCACCG | | | |
| | | RLAKTYETTLEKCCAAA | UGGCCACCCUGAGA | | | |
| | | DPHECYAKVFDEFKPLV | GAAACUUACGGCGA | | | |
| | | EEPQNLIKQNCELFEQL | GAUGGCCGACUGCU | | | |
| | | GEYKFQNALLVRYTKKV | GCGCCAAGCAGGAG | | | |
| | | PQVSTPTLVEVSRNLGK | CCAGAGCGGAACGA | | | |
| | | VGSKCCKHPEAKRMPC | GUGCUUCCUCCAGC | | | |
| | | AEDYLSVVLNQLCVLHE | ACAAGGACGACAAC | | | |
| | | KTPVSDRVTKCCTESLV | CCUAACCUGCCUAG | | | |
| | | NRRPCFSALEVDETYVP | ACUGGUAAGGCCU | | | |
| | | KEFNAETFTFHADICTLS | GAGGUGGACGUGA | | | |
| | | EKERQIKKQTALVELVK | UGUGUACCGCCUUC | | | |
| | | HKPKATKEQLKAVMDD | CACGACAACGAGGA | | | |
| | | FAAFVEKCCKADDKETC | GACAUUCCUGAAGA | | | |
| | | FAEEGKKLVAASQAAL | AGUACCUGUACGAG | | | |
| | | GLGGGSAPTSSSTKKTQ | AUCGCCAGAAGACA | | | |
| | | LQLEHLLLDLQMILNGI | CCCUUACUUCUACG | | | |
| | | NNYKNPKLTRMLTFKFY | CCCCUGAGUUGCUG | | | |
| | | MPKKATELKHLQCLEEE | UUCUUCGCGAAGA | | | |
| | | LKPLEEALNLAPSKNFHL | GAUACAAGGCCGCC | | | |
| | | RPRDLISDINVIVLELKG | UUCACCGAGUGCUG | | | |
| | | SETTFMCEYADETATIV | CCAGGCCGCCGAUA | | | |
| | | EFLNRWITFCQSIISTLT | AGGCCGCGUGCCUG | | | |
| | | | CUGCCUAAGCUGGA | | | |
| | | | CGAGCUGAGAGACG | | | |
| | | | AGGGCAAGGCAUCC | | | |
| | | | AGCGCUAAGCAGAG | | | |
| | | | ACUGAAGUGCGCCA | | | |
| | | | GCCUGCAGAAGUUC | | | |
| | | | GGAGAGAGAGCUU | | | |
| | | | UCAAGGCGUGGGC | | | |
| | | | AGUGGCUAGAUUG | | | |
| | | | AGCCAAAGAUUCCC | | | |
| | | | UAAGGCAGAAUUC | | | |
| | | | GCUGAGGUGAGCA | | | |
| | | | AGCUCGUGACUGAC | | | |
| | | | CUGACCAAGGUGCA | | | |
| | | | UACAGAGUGCUGU | | | |
| | | | CACGGCGACCUGCU | | | |
| | | | GGAGUGCGCCGACG | | | |
| | | | ACAGAGCCGACCUG | | | |
| | | | GCCAAGUACAUCUG | | | |
| | | | CGAGAACCAGGACA | | | |
| | | | GCAUCAGCAGCAAG | | | |
| | | | CUGAAGGAGUGUU | | | |
| | | | GUGAGAAGCCUCUA | | | |
| | | | UUGGAGAAGAGUC | | | |
| | | | ACUGCAUUGCCGAG | | | |
| | | | GUGGAGAACGACGA | | | |
| | | | GAUGCCUGCGGAUC | | | |
| | | | UGCCAAGCUUGGCG | | | |
| | | | GCCGACUUCGUGGA | | | |
| | | | GAGCAAGGACGUG | | | |
| | | | UGCAAGAACUACGC | | | |
| | | | CGAGGCCAAGGACG | | | |
| | | | UUUUCCUGGGCAU | | | |
| | | | GUUCCUCUACGAGU | | | |
| | | | ACGCACGCAGACAU | | | |
| | | | CCAGACUACAGCGU | | | |
| | | | GGUGCUGCUGCUG | | | |
| | | | AGACUGGCUAAGAC | | | |
| | | | AUACGAAACUACCC | | | |
| | | | UGGAGAAGUGCUG | | | |
| | | | CGCAGCGGCGGACC | | | |
| | | | CUCACGAGUGUUAC | | | |
| | | | GCCAAGGUGUUCG | | | |
| | | | ACGAGUUCAAGCCU | | | |
| | | | CUGGUGGAGGAGC | | | |
| | | | CUCAGAACCUGAUC | | | |
| | | | AAGCAGAACUGUGA | | | |
| | | | GCUGUUCGAGCAGC | | | |
| | | | UCGGCGAGUACAAG | | | |
| | | | UUCCAGAACGCCCU | | | |
| | | | GUUGGUCCGCUACA | | | |
| | | | CCAAGAAGGUGCCU | | | |
| | | | CAAGUAAGUACCCC | | | |
| | | | UACCCUGGUAGAG | | | |
| | | | GUUAGUAGAAACC | | | |
| | | | UGGGCAAGGUGGG | | | |
| | | | CAGCAAGUGCUGUA | | | |
| | | | AGCACCCAGAAGCU | | | |
| | | | AAGAGGAUGCCUU | | | |
| | | | GCGCCGAGGACUAC | | | |
| | | | CUGUCCGUUGUGC | | | |
| | | | UGAACCAGCUGUGC | | | |
| | | | GUGCUGCACGAGAA | | | |
| | | | GACCCCUGUGAGCG | | | |
| | | | ACAGAGUGACAAAG | | | |
| | | | UGUUGCACCGAGA | | | |
| | | | GCUUAGUGAAUAG | | | |
| | | | AAGACCUUGCUUCA | | | |
| | | | GCGCCCUGGAAGUU | | | |
| | | | GACGAAACUUACGU | | | |
| | | | GCCUAAGGAGUUCA | | | |
| | | | ACGCCGAGACUUUC | | | |
| | | | ACAUUCCACGCCGA | | | |
| | | | CAUUUGCACUCUGA | | | |
| | | | GCGAGAAGGAGAG | | | |
| | | | ACAGAUCAAGAAGC | | | |
| | | | AGACCGCCCUCGUA | | | |
| | | | GAGUUGGUCAAGC | | | |
| | | | ACAAGCCGAAGGCA | | | |
| | | | ACCAAGGAACAGCU | | | |
| | | | UAAGGCCGUGAUG | | | |
| | | | GACGACUUCGCAGC | | | |
| | | | CUUCGUCGAGAAG | | | |
| | | | UGUUGUAAGGCCG | | | |
| | | | ACGACAAGGAGACU | | | |
| | | | UGCUUCGCAGAGG | | | |
| | | | AAGGCAAGAAGUU | | | |
| | | | GGUAGCCGCCUCUC | | | |
| | | | AGGCUGCCUUGGG | | | |
| | | | ACUCGGUGGCGGCA | | | |
| | | | GCGCCCCUACCAGC | | | |
| | | | AGCUCCACAAAGAA | | | |
| | | | GACGCAGCUGCAGC | | | |
| | | | UGGAGCACCUGCUA | | | |
| | | | CUAGACUUACAGAU | | | |
| | | | GAUCCUGAACGGCA | | | |
| | | | UCAACAACUACAAG | | | |
| | | | AACCCGAAGCUCAC | | | |
| | | | CAGAAUGCUGACAU | | | |
| | | | UCAAGUUCUACAUG | | | |
| | | | CCUAAGAAGGCCAC | | | |
| | | | UGAGCUGAAGCACC | | | |
| | | | UUCAGUGCCUGGA | | | |
| | | | GGAGGAACUUAAG | | | |
| | | | CCACUGGAAGAGGC | | | |
| | | | CCUGAACCUGGCCC | | | |
| | | | CUAGUAAGAAUUU | | | |
| | | | CCACCUCAGACCUC | | | |
| | | | GCGACCUUAUUAGC | | | |
| | | | GACAUCAACGUGAU | | | |
| | | | CGUGCUCGAAUUAA | | | |
| | | | AGGGCUCCGAGACA | | | |
| | | | ACAUUCAUGUGCGA | | | |
| | | | AUACGCCGACGAGA | | | |
| | | | CAGCCACCAUCGUG | | | |
| | | | GAGUUCCUGAACCG | | | |
| | | | CUGGAUCACCUUCU | | | |
| | | | GCCAGAGCAUCAUC | | | |
| | | | UCUACUCUGACC | | | |

In an embodiment, a LNP composition described herein comprises a polynucleotide encoding an E1-2 molecule. In an embodiment, the E1-2 molecule further comprises a targeting moiety, e.g., a T regulatory cell targeting moiety or a tissue-specific targeting moiety.

In an embodiment, the E1-2 molecule further comprises a tissue targeting moiety. In an embodiment, the tissue-specific targeting moiety binds to ROS-CII, EDA, EDB, TnC A1, SyETP, GLUT-2, GD2, FAP, VCAM or MADCAM.

In an embodiment, an LNP composition described herein comprises a polynucleotide encoding an EL-2 molecule. In an embodiment, the EL-2 molecule further comprises a T regulatory cell targeting moiety. In an embodiment, the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof. In an embodiment, the T regulatory cell targeting moiety binds to a molecule present on a T regulatory cell. In an embodiment, the T regulatory cell targeting moiety comprises an antibody molecule that binds to CTLA-4, GITR, TLR8, or Nrp11.

In an embodiment, the T regulatory cell targeting moiety binds to CTLA-4. In an embodiment, the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17. In an embodiment, the targeting moiety comprises the amino acid sequence of SEQ ID NO: 17.

In an embodiment, the IL-2 molecule further comprises a T regulatory cell targeting moiety that binds to CTLA-4. In an embodiment, the IL-2 molecule comprising the targeting moiety comprises that binds to CTLA-4 comprises an amino acid sequence having at least 85%, 90%, 95%, 96%, 97%, 98%, 99% or 100% identity to an amino acid sequence provided in Table 2A. In an embodiment, the IL-2 molecule comprising the targeting moiety comprises that binds to CTLA-4 comprises an amino acid sequence provided in Table 2A.

In an embodiment, the IL-2 molecule further comprises a T regulatory cell targeting moiety that binds to CTLA-4. In an embodiment, the IL-2 molecule comprising the T regulatory cell targeting moiety that binds to CTLA-4 comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20. In an embodiment, the IL-2 molecule comprising the targeting moiety comprises that binds to CTLA-4 comprises the amino acid sequence of SEQ ID NO: 18. In an embodiment, the IL-2 molecule comprising the targeting moiety comprises that binds to CTLA-4 comprises the amino acid sequence of SEQ ID NO: 19. In an embodiment, the IL-2 molecule comprising the targeting moiety comprises that binds to CTLA-4 comprises the amino acid sequence of SEQ ID NO: 20.

In an embodiment, an LNP composition described herein comprises a first polynucleotide encoding an IL-2 molecule. In an embodiment, the IL-2 molecule comprises a T regulatory cell moiety that binds to CTLA-4. In an embodiment, the first polynucleotide encoding the IL-2 molecule comprising a T regulatory cell moiety that binds to CTLA-4 comprises a nucleotide sequence having at least 85%, 90%, 95%, 96%, 97%, 98%, 99% or 100% identity to a nucleic acid sequence provide in Table 2A. In an embodiment, the first polynucleotide encoding the IL-2 molecule comprising a T regulatory cell moiety that binds to CTLA-4 comprises a nucleotide sequence having at least 85%, 90%, 95%, 96%, 97%, 98%, 99% or 100% identity to SEQ ID NO: 21, SEQ ID NO: 22 or SEQ ID NO: 23.

TABLE 2A
Exemplary CTLA-4 binder sequences and IL-2 CTLA-4 sequences

| SEQ | | |
|---|---|---|
| ID | | |
| NO | Sequence information | Sequence |
| 17 | CTLA4_03 (Human, heavy | EVQLVQTGGGLSQFGESLRLSCAVSGFNVSNNYM |
| | chain only antibody binder | SWVRQAPGKGLEWVSIIYSGGGTHYADSVKGRFT |
| | to CTLA4, derived from | ISRDNSKNTLFLQMNSLRAEDTAVYYCARAVPVP |
| | HCAB mice) | HGTDIWGQGTMVTVSS |
| 18 | Leader-CTLA4.03- | MPLLLLLPLLWAGALAEVQLVQTGGGLSQFGESL |
| | (G4S)3-HSA-(G3S)- | RLSCAVSGFNVSNNYMSWVQAPGKGLEWVSIIYSGG |
| | (Human IL-2)-V5 tag | GTHYADSVKGRFTISRDNSKNTLFLQMNSLRAEDTAV |
| | | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISNI |
| | | NVIVLELKGSETTFMCEYADETATIVEFLNRWITF |
| | | CQSIISTLTGKPIPNPLLGLDST |
| 21 | LeaderCTLA4.03- | AUGCCUCUGUUACUGCUGCUCCCACUUCUGUGGGCC |
| | (G4S)3-HSA-(G3S)- | GGCGCUCUGGCUGAGGUGCAGCUUGUGCAGACCGGCGG |
| | (Human IL-2)-V5 tag | AGGUCUGUCACAGUUCGGCGAAUCCCUAAGGCUCUCUU |
| | | GCGCCGUGUCUGGUUUCAACGUGAGCAACAACUAUAUG |
| | | AGUUGGGUUCGCCAGGCUCCAGGCAAGGGUCUGGAGUG |
| | | GGUGUCCAUUAUCUAUUCCGGCGGUGGCACACAUUACG |
| | | CCGACUCAGUCAAGGGUAGAUUCACUAUCAGUAGAGAC |
| | | AACUCAAAGAACACUCUUUUCCUACAAAUGAACAGCUU |
| | | GCGGGCAGAGGAUACGGCCGUGUAUUAUUGUGCCCGAG |
| | | CUGUCCCAGUGCCUCACGGAACAGAUAUUUGGGGACAA |
| | | GGUACAAUGGUGACCGUCUCUUCCGGCGGAGGAGGCUC |
| | | UGGAGGCGGAGGAUCUGGCGGAGGCGGCAGCGCCCAUA |
| | | AGAGCGAGGUAGCCCAUAGAUUCAAGGACCUUGGCGAA |
| | | GAGAACUUCAAGGCCUUAGUUCUCAUCGCCUUCGCCCA |
| | | AUACUUGCAGCAGUGUCCAUUCGAAGAUCACGUUAAGC |
| | | UUGUGAACGAGGUGACCGAAUUCGCUAAGACGUGCGUG |
| | | GCCGACGAGUCCGCUGAGAAUUGUGAUAAGUCACUGCA |
| | | CACUUUGUUCGGCGACAAGCUGUGCACCGUCGCCACCC |
| | | UGCGCGAAACAUACGGCGAAAUGGCGGACUGUUGUGCU |
| | | AAGCAGGAGCCAGAACGGAACGAGUGUUUCCUGCAGCA |
| | | UAAGGACGACAAUCCAAACCUCCCUAGGUUGGUGCGCC |
| | | CAGAAGUGGACGUCAUGUGUACCGCUUUCCACGAUAAC |
| | | GAGGAGACAUUCCUUAAGAAGUAUCUGUACGAGAUUGC |
| | | ACGGCGGCACCCUUACUUCUACGCCCCUGAGCUCCUGUU |
| | | CUUCGCAAAGCGGUAUAAGGCUGCAUUCACCGAGUGUU |
| | | GCCAGGCGGCAGACAAGGCCGCCUGCUUGCUCCCUAAG |
| | | CUUGACGAACUUAGGGACGAGGGAAAGGCUAGUAGCGC |
| | | CAAGCAGAGACUCAAGUGUGCAUCGCUACAGAAGUUCG |
| | | GAGAGAGAGCUUUCAAGGCGUGGGCUGUCGCGCGCCUG |
| | | UCCCAGAGGUUCCCAAAGGCCGAAUUCGCCGAGGUGAG |
| | | CAAGUUAGUGACAGAUCUCACCAAGGUGCACACUGAGU |
| | | GUUGCCACGGCGACUUAUUGGAGUGUGCAGACGAUCGG |
| | | GCUGACUUGGCAAAGUACAUCUGUGAGAACCAGGACUC |
| | | AAUUUCCAGUAAGCUGAAGGAGUGUUGCGAGAAGCCAC |
| | | UCUUGGAGAAGUCCCAUUGUAUAGCCGAAGUGGAGAAC |
| | | GACGAGAUGCCGGCUGACCUGCCGUCACUGGCAGCUGA |
| | | UUUCGUAGAGUCGAAGGACGUGUGUAAGAACUACGCUG |
| | | AAGCUAAGGACGUAUUCCUGGGAAUGUUCCUUUACGAG |
| | | UACGCCCGACGGCAUCCAGACUACUCAGUAGUGCUGCU |
| | | UCUGAGACUGGCCAAGACUUACGAGACUACCCUGGAGA |
| | | AGUGUUGUGCAGCGGCCGAUCCACACGAGUGUUACGCC |
| | | AAGGUGUUCGACGAAUUCAAGCCUCUGGUGGAGGAGCC |
| | | GCAGAACCUGAUCAAGCAGAAUUGCGAACUGUUCGAGC |
| | | AGUUGGGAGAAUAUAAGUUCCAGAACGCACUGCUAGUG |
| | | CGGUAUACCAAGAAGGUCCCACAGGUAUCCACUCCGAC |
| | | ACUGGUGGAAGUGUCCAGGAACCUCGGCAAGGUGGGAA |
| | | GCAAGUGCUGUAAGCACCCGGAGGCUAAGCGGAUGCCG |
| | | UGCGCCGAAGACUACCUCAGCGUGGUCCUUAAUCAGCU |
| | | GUGCGUGCUGCACGAGAAGACCCCAGUAAGUGACCGGG |
| | | UUACGAAGUGCUGUACUGAAUCACUUGUGAACAGAAGA |
| | | CCUUGUUUCUCCGCCCUCGAGGUGGACGAAACGUACGU |
| | | UCCGAAGGAAUUCAACGCCGAAACAUUCACCUUCCACG |
| | | CUGACAUUUGUACGCUUUCCGAGAAGGAGAGACAAAUC |
| | | AAGAAGCAGACUGCCCUGGUGGAAUUAGUGAAGCACAA |
| | | GCCGAAGGCCACAAAGGAGCAGCUGAAGGCAGUUAUGG |
| | | ACGACUUCGCUGCAUUCGUAGAGAAGUGCUGCAAGGCA |
| | | GACGACAAGGAAACUUGCUUCGCAGAGGAGGGAAAGAA |
| | | GCUCGUUGCUGCGUCUCAGGCGGCUCUGGGACUGGGAG |
| | | GAGGAUCCGCUCCUACAAGCAGUUCCACAAAGAAGACC |
| | | CAGCUCCAGCUUGAACAUCUGCUUCUCGACCUGCAAAU |
| | | GAUCCUCAACGGUAUCAAUAAUUACAAGAAUCCUAAGU |
| | | UGACUCGCAUGCUGACUUUCAAGUUCUAUAUGCCAAAG |
| | | AAGGCCACUGAGUUGAAGCACCUGCAGUGCCUCGAGGA |
| | | AGAACUGAAGCCUCUGGAGGAAGUCUUAAACCUGGCAC |
| | | AAAGCAAGAACUUCCAUCUGAGGCCUAGGGACCUCAUU |
| | | UCCAAUAUCAACGUUAUUGUGCUGGAGCUCAAGGGUAG |
| | | CGAGACGACAUUCAUGUGCGAGUACGCAGACGAGACAG |
| | | CGACUAUCGUCGAGUUCCUAAAUAGGUGGAUAACGUUC |
| | | UGCCAGUCAAUCAUCUCCACUCUGACCGGCAAGCCGAU |
| | | CCCUAACCCGUUGCUUGGAUUGGAUUCUACA |
| 19 | Leader-CTLA4.03- | MPLLLLLPLLWAGALAEVQLVQTGGGLSQFGES |
| | (G4S)3-HSA-(G3S)- | LRLSCAVSGFNVSNNYMSWVRQAPGKGLEWVSIIYSG |
| | (Human IL-2 D20T)-V5 | GGTHYADSVKGRFTISRDNSKNTLFLQMNSLRAEDT |
| | tag | AVYYCARAVPVPHGTDIWGQGTMVTVSSGGGGSGG |
| | | GGSGGGGSAHKSEVAHRFKDLGEENFKALVLIAF |
| | | AQYLQQCPFEDHVKLVNEVTEFAKTCVADESAEN |
| | | CDKSLHTLFGDKLCTVATLRETYGEMADCCAKQ |
| | | EPERNECFLQHKDDNPNLPRLVRPEVDVMCTAFH |
| | | DNEETFLKKYLYEIARRHPYFYAPELLFFAKRYKA |
| | | AFTECCQAADKAACLLPKLDELRDEGKASSAKQR |
| | | LKCASLQKFGERAFKAWAVARLSQRFPKAEFAEV |
| | | SKLVTDLTKVHTECCHGDLLECADDRADLAKYIC |
| | | ENQDSISSKLKECCEKPLLEKSHCIAEVENDEMPA |
| | | DLPSLAADFVESKDVCKNYAEAKDVFLGMFLYE |
| | | YARRHPDYSVVLLLRLAKTYETTLEKCCAAADPH |
| | | ECYAKVFDEFKPLVEEPQNLIKQNCELFEQLGEYK |
| | | FQNALLVRYTKKVPQVSTPTLVEVSRNLGKVGSK |
| | | CCKHPEAKRMPCAEDYLSVVLNQLCVLHEKTPVS |
| | | DRVTKCCTESLVNRRPCFSALEVDETYVPKEFNA |
| | | ETFTFHADICTLSEKERQIKKQTALVELVKHKPKA |
| | | TKEQLKAVMDDFAAFVEKCCKADDKETCFAEEG |
| | | KKLVAASQAALGLGGGSAPTSSSTKKTQLQLEHL |
| | | LLTLQMILNGINNYKNPKLTRMLTFKFYMPKKAT |
| | | ELKHLQCLEEELKPLEEVLNLAQSKNFHLRPRDLI |
| | | SNINVIVLELKGSETTFMCEYADETATIVEFLNRWI |
| | | TFCQSIISTLTGKPIPNPLLGLDST |
| 22 | Leader-CTLA4.03- | AUGCCUCUGCUGCUCUUACUGCCGCUGCUUUGGGCA |
| | (G4S)3-HSA-(G3S)- | GGAGCUCUGGCCGAGGUCCAGCUAGUGCAGACAGGCGG |
| | (Human IL-2 D20T)-V5 | UGGACUCAGCCAGUUCGGUGAGUCACUGCGUCUAUCCU |
| | tag | GCGCGGUUAGUGGCUUCAACGUGUCCAACAAUUAUAUG |
| | | AGUUGGGUGAGACAAGCACCUGGCAAGGGCCUUGAGUG |
| | | GGUGUCCAUUAUAUAUAGCGGUGGCGGCACCCACUACG |
| | | CUGACUCGGUGAAGGGUCGAUUCACAAUUAGCAGAGAC |
| | | AAUUCCAAGAAUACUCUGUUCCUGCAGAUGAACUCUCU |
| | | CAGGGCGGAGGACACCGCCGUGUACUACUGUGCCCGUG |
| | | CGGUGCCGGUCCCUCACGGUACAGACAUCUGGGGCCAG |
| | | GGUACCAUGGUCACCGUGUCAUCCGGAGGAGGCGGUUC |
| | | UGGUGGAGGAGGAAGUGGAGGCGGCGGCAGUGCACAUA |
| | | AGAGCGAGGUGGCCCAUAGAUUCAAGGAUUUAGGAGAG |
| | | GAGAACUUCAAGGCACUGGUCUUGAUCGCCUUCGCACA |
| | | AUAUCUUCAGCAGUGUCCGUUCGAAGAUCACGUGAAGC |
| | | UCGUCAACGAGGUGACUGAGUUCGCCAAGACCUGCGUG |
| | | GCAGACGAGUCGGCUGAGAAUUGUGAUAAGAGUCUGCA |
| | | CACCCUCUUCGGAGAUAAGCUGUGUACCGUGGCCACUC |
| | | UGAGGGAGACAUACGGCGAGAUGGCUGACUGUUGUGCA |
| | | AAGCAGGAGCCAGAGAGGAACGAGUGUUUCUUGCAGCA |
| | | UAAGGACGACAACCCAAAUCUGCCUCGUCUGGUUAGAC |
| | | CUGAAGUGGACGUAAUGUGCACCGCUUUCCACGACAAC |
| | | GAGGAGACGUUCCUGAAGAAGUAUCUGUACGAGAUUGC |
| | | UAGAAGGCACCCAUACUUCUACGCACCAGAGCUGUUGU |
| | | UCUUCGCUAAGAGAUACAAGGCCGCUUUCACCGAGUGC |
| | | UGCCAAGCAGCAGACAAGGCUGCUUGCCUGCUCCCUAA |
| | | GCUGGACGAGCUGAGAGACGAGGGUAAGGCUUCAUCAG |
| | | CCAAGCAAAGAUUGAAGUGUGCAUCACUGCAGAAGUUC |
| | | GGCGAAAGAGCAUUCAAGGCUUGGGCCGUCGCCAGGCU |
| | | GUCUCAGAGGUUCCCGAAGGCCGAGUUCGCUGAAGUGU |
| | | CUAAGUUAGUGACCGAUCUAACAAAGGUGCACACAGAG |
| | | UGUUGCCACGGAGAUCUGCUUGAGUGCGCCGACGAUCG |
| | | AGCCGAUCUUGCAAAGUACAUCUGUGAGAACCAGGAUU |
| | | CCAUCUCCUCUAAGUUGAAGGAGUGCUGUGAGAAGCCA |
| | | CUUCUCGAGAAGUCUCACUGCAUCGCAGAAGUCGAGAA |
| | | CGACGAAAUGCCAGCUGACCUGCCGAGCUUGGCCGCAG |
| | | AUUUCGUCGAGAGUAAGGACGUUUGCAAGAAUUACGCU |
| | | GAGGCUAAGGACGUCUUCCUGGGCAUGUUCUUGUACGA |
| | | AUACGCCAGGAGGCACCCGGAUUACUCAGUGGUACUUU |
| | | UACUGCGACUGGCUAAGACCUACGAAACGACACUGGAG |
| | | AAGUGCUGCGCAGCCGCCGAUCCUCACGAGUGCUACGC |
| | | UAAGGUUUUCGACGAGUUCAAGCCGCUCGUGGAAGAAC |
| | | CUCAGAACUUAAUCAAGCAGAAUUGUGAGCUAUUCGAA |
| | | CAGCUCGGAGAGUAUAAGUUCCAGAACGCCCUCCUGGU |
| | | GCGGUACACAAAGAAGGUUCCACAGGUCAGCACCCCGA |
| | | CCCUCGUUGAGGUGAGCCGCAACCUCGGCAAGGUUGGC |
| | | UCUAAGUGUUGUAAGCAUCCGGAAGCGAAGCGCAUGCC |
| | | UUGUGCAGAAGAUUAUCUUUCAGUUGUGUUGAACCAGC |
| | | UCUGUGUCCUGCACGAGAAGACCCCAGUUUCCGACCGC |
| | | GUUACCAAGUGUUGCACAGAAAGCUUGGUCAACCGGAG |
| | | GCCUUGCUUCAGCGCAUUGGAGGUAGACGAAACCUACG |
| | | UCCCUAAGGAAUUCAACGCCGAAACAUUCACGUUCCAC |
| | | GCGGACAUUUGCACACUCUCCGAGAAGGAACGCCAGAU |
| | | UAAGAAGCAGACUGCCUUGGUUGAGCUGGUAAAGCACA |
| | | AGCCUAAGGCGACUAAGGAGCAGCUAAAGGCAGUUAUG |
| | | GACGACUUCGCUGCAUUCGUUGAGAAGUGCUGUAAGGC |
| | | CGACGAUAAGGAAACCUGCUUCGCCGAGGAAGGAAAGA |
| | | AGCUUGUUGCCGCUAGCCAGGCCGCACUGGGAUUGGGU |
| | | GGAGGCAGCGCACCAACUUCCUCUAGCACCAAGAAGAC |
| | | UCAAUUACAACUGGAACAUCUCUUACUAACACUCCAAA |
| | | UGAUACUGAACGGCAUAAACAACUAUAAGAACCCGAAG |
| | | UUGACCAGAAUGUUAACCUUCAAGUUCUACAUGCCUAA |
| | | GAAGGCUACGGAACUGAAGCACCUUCAGUGCCUAGAGG |
| | | AGGAGCUCAAGCCUCUCGAGGAGGUUCUGAACCUCGCA |
| | | CAGAGUAAGAAUUUCCACCUGCGCCCAAGGGACCUGAU |
| | | UUCCAAUAUUAACGUGAUCGUCCUUGAACUUAAGGGCA |
| | | GCGAAACCACUUUCAUGUGCGAAUACGCGGACGAAACU |
| | | GCUACCAUAGUUGAGUUCCUUAACCGCUGGAUCACAUU |
| | | CUGCCAGUCAAUCAUCUCUACAUUGACCGGAAAGCCGA |
| | | UUCCGAACCCACUCCUGGGACUUGAUAGCACA |
| 20 | Leader-CTLA4.03- | MPLLLLLPLLWAGALAEVQLVQTGGGLSQFGESL |
| | (G4S)3-HSA-(G3S)- | RLSCAVSGENVSNNYMSWVRQAPGKGLEWVSIIYSGG |
| | (Human IL-2 N88D)-V5 | GTHYADSVKGRFTISRDNSKNTLFLQMNSLRAEDTAV |
| | tag | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISDI |
| | | NVIVLELKGSETTFMCEYADETATIVEFLNRWITF |
| | | CQSIISTLTGKPIPNPLLGLDS |
| 23 | Leader-CTLA4.03- | AUGCCUCUGCUCCUGCUUCUUCCUCUGCUCUGGGCU |
| | (G4S)3-HSA-(G3S)- | GGCGCCCUAGCCGAGGUGCAGCUGGUGCAGACCGGCGG |
| | (Human IL-2 N88D)-V5 | CGGCCUGAGCCAGUUCGGCGAGAGCCUGAGACUGAGCU |
| | tag | GCGCCGUGAGCGGCUUCAACGUGAGCAACAACUACAUG |
| | | AGCUGGGUGAGACAGGCCCCUGGCAAGGGCCUGGAGUG |
| | | GGUGAGCAUCAUCUACAGUGGCGGAGGUACCCACUACG |
| | | CCGACAGCGUGAAGGGCAGAUUCACCAUCAGCAGAGAC |
| | | AACAGCAAGAACACCCUGUUCCUGCAGAUGAACAGCCU |
| | | CAGGGCCGAGGACACCGCCGUGUACUACUGCGCCAGAG |
| | | CCGUGCCUGUGCCUCACGGCACCGACAUCUGGGGCCAG |
| | | GGCACCAUGGUGACCGUGAGUAGCGGUGGAGGCGGAAG |
| | | CGGAGGCGGUGGCUCUGGAGGUGGCGGCAGCGCCCACA |
| | | AGAGCGAGGUGGCCCACAGAUUCAAGGACCUGGGCGAG |
| | | GAGAACUUCAAGGCCCUGGUGCUGAUCGCCUUCGCCCA |
| | | GUACCUGCAGCAGUGCCCUUUCGAGGACCACGUGAAGC |
| | | UGGUGAACGAGGUGACCGAGUUCGCCAAGACCUGCGUG |
| | | GCCGACGAGAGCGCCGAGAACUGCGACAAGAGCCUGCA |
| | | CACACUCUUCGGCGACAAGCUGUGCACCGUGGCCACCCU |
| | | GAGAGAGACUUACGGCGAGAUGGCCGACUGCUGCGCCA |
| | | AGCAGGAACCAGAGAGGAACGAGUGCUUCCUUCAGCAC |
| | | AAGGACGACAACCCUAACCUGCCUAGACUGGUUCGGCC |
| | | UGAGGUGGACGUGAUGUGCACUGCCUUCCACGACAACG |
| | | AGGAGACGUUCCUGAAGAAGUACCUGUACGAGAUCGCC |
| | | AGAAGACACCCUUACUUCUACGCCCCUGAGCUUCUGUU |
| | | CUUCGCAAAGAGAUACAAGGCCGCCUUCACCGAGUGCU |
| | | GCCAGGCCGCCGACAAGGCUGCCUGCCUCCUGCCAAAGC |
| | | UGGACGAGCUGAGAGACGAGGGCAAGGCCAGCUCUGCG |
| | | AAGCAGAGGCUGAAGUGCGCCAGCCUGCAGAAGUUCGG |
| | | AGAAAGAGCUUUCAAGGCCUGGGCCGUGGCCCGCUUGU |
| | | CUCAAAGAUUCCCUAAGGCGGAGUUCGCAGAAGUGAGC |
| | | AAGCUGGUCACCGACCUGACCAAGGUGCAUACUGAGUG |
| | | UUGCCACGGCGACCUGCUGGAGUGCGCCGACGACAGAG |
| | | CCGACCUGGCCAAGUACAUCUGCGAGAACCAGGACUCU |
| | | AUUAGUAGCAAGUUGAAGGAGUGUUGCGAGAAGCCGCU |
| | | ACUCGAGAAGAGUCACUGCAUUGCGGAGGUUGAGAACG |
| | | ACGAGAUGCCUGCCGACCUCCCAAGCCUGGCAGCCGAU |
| | | UUCGUGGAGAGCAAGGACGUGUGCAAGAAUUACGCUGA |
| | | GGCUAAGGACGUUUUCCUGGGCAUGUUCUUAUACGAAU |
| | | ACGCUAGACGGCACCCAGACUACAGCGUGGUCCUCCUCC |
| | | UGCGGCUUGCAAAGACGUACGAAACUACCCUGGAGAAG |
| | | UGCUGCGCGGCUGCUGACCCUCACGAGUGCUACGCCAA |
| | | GGUGUUCGACGAGUUCAAGCCUCUCGUGGAGGAGCCUC |
| | | AGAACCUGAUCAAGCAGAAUUGCGAGCUGUUCGAGCAG |
| | | UUAGGUGAGUACAAGUUCCAGAACGCCCUGCUUGUGCG |
| | | CUACACCAAGAAGGUGCCUCAGGUUUCCACCCCUACCCU |
| | | GGUAGAGGUGUCCAGAAACCUGGGCAAGGUGGGCAGCA |
| | | AGUGUUGCAAGCAUCCAGAAGCUAAGAGAAUGCCUUGC |
| | | GCCGAAGAUUACCUGUCUGUCGUGCUGAACCAGCUGUG |
| | | CGUGCUGCACGAGAAGACCCCUGUGAGCGACAGAGUGA |
| | | CCAAGUGCUGUACAGAAUCCCUAGUAAAUAGAAGACCU |
| | | UGCUUCUCCGCCCUAGAGGUGGACGAAACCUACGUGCC |
| | | UAAGGAGUUCAACGCCGAGACUUUCACGUUCCACGCGG |
| | | AUAUUUGUACACUCAGCGAGAAGGAGAGACAGAUCAAG |
| | | AAGCAGACCGCACUUGUGGAGCUCGUCAAGCACAAGCC |
| | | AAAGGCCACCAAGGAGCAACUCAAGGCCGUGAUGGACG |
| | | ACUUCGCAGCCUUCGUCGAGAAGUGUUGUAAGGCUGAC |
| | | GAUAAGGAAACCUGCUUCGCUGAGGAAGGCAAGAAGCU |
| | | UGUGGCCGCCAGUCAGGCUGCUCUUGGUCUUGGCGGCG |
| | | GAUCAGCACCUACCAGCAGCUCUACAAAGAAGACGCAG |
| | | CUGCAACUGGAGCACUUGCUGCUUGAUCUGCAAAUGAU |
| | | CCUGAACGGCAUCAACAAUUACAAGAACCCUAAGCUGA |
| | | CCAGAAUGCUGACCUUCAAGUUCUACAUGCCUAAGAAG |
| | | GCCACAGAGCUGAAGCAUUUACAGUGCCUGGAGGAGGA |
| | | GCUUAAGCCACUUGAAGAGGUGCUCAAUCUGGCCCAGU |
| | | CUAAGAACUUCCACCUCAGGCCUCGCGAUCUGAUUAGC |
| | | GACAUCAACGUAAUCGUGCUGGAACUUAAGGGAUCUGA |
| | | AACUACGUUCAUGUGCGAGUACGCCGACGAAACAGCCA |
| | | CCAUCGUGGAGUUCCUGAACCGAUGGAUCACCUUCUGC |
| | | CAGAGUAUUAUUUCAACCCUCACCGGCAAGCCUAUCCC |
| | | UAACCCACUGCUCGGACUCGACAGCACC |

### GM-CSF Molecule

Granulocyte-macrophage colony stimulating factor (GM-CSF) is a cytokine which is secreted by many cells including, macrophages, T cells, mast cells, natural killer cells, endothelial cells and fibroblasts. GM-CSF is also known as colony stimulating factor 2 (CSF2). GM-CSF can stimulate stem cells to produce granulocytes (e.g., neutrophils) and monocytes, which can mature into macrophages and dendritic cells (DCs). GM-CSF can also increase DC maturation, function and recruitment.

In an aspect, the disclosure provides an LNP composition comprising a polynucleotide (e.g., mRNA) encoding a GM-CSF molecule, e.g., as described herein. In an embodiment, the GM-CSF molecule comprises a naturally occurring GM-CSF molecule, a fragment of a naturally occurring GM-CSF molecule, or a variant thereof. In an embodiment, the GM-CSF molecule comprises a variant of a naturally occurring GM-CSF molecule (e.g., a GM-CSF variant, e.g., as described herein), or a fragment thereof.

In an embodiment, the LNP composition comprising a polynucleotide encoding a GM-CSF molecule can be administered alone or in combination with an LNP composition comprising a polynucleotide encoding an IL-2 molecule. In an embodiment, the LNP composition comprising the GM-CSF molecule and the LNP composition comprising the IL-2 molecule are administered sequentially. In an embodiment, the LNP composition comprising the GM-CSF molecule is administered first and the LNP composition comprising the IL-2 molecule is administered second. In an embodiment, the LNP composition comprising the GM-CSF molecule is administered second and the LNP composition comprising the IL-2 molecule is administered first. In an embodiment, the LNP composition comprising the GM-CSF molecule and the LNP composition comprising the IL-2 molecule are administered simultaneously, e.g., substantially simultaneously.

In an embodiment, the LNP composition comprising the GM-CSF molecule and the LNP composition comprising the IL-2 molecule are in the same or different compositions.

In an embodiment, the GM-CSF molecule comprises an amino acid sequence having at least 85%, 90%, 95%, 96%, 97%, 98%, 99% or 100% identity to the amino acid sequence of a GM-CSF molecule provided in Table 3A or 3B. In an embodiment, the GM-CSF molecule comprises of a GM-CSF molecule provided in Table 3A or 3B. In an embodiment, the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41 or SEQ ID NO: 43. In an embodiment, the GM-CSF molecule comprises the amino acid sequence of SEQ ID NO: 14. In an embodiment, the GM-CSF molecule comprising the amino acid sequence of SEQ ID NO: 14 further comprises a leader sequence. In an embodiment, the GM-CSF molecule comprises the amino acid sequence of SEQ ID NO: 188. In an embodiment, the GM-CSF molecule comprises the amino acid sequence of SEQ ID NO: 188 without the leader sequence. In an embodiment, the GM-CSF molecule comprises the amino acid sequence of SEQ ID NO: 39. In an embodiment, the GM-CSF molecule comprises the amino acid sequence of SEQ ID NO: 39 without the leader sequence. In an embodiment, the GM-CSF molecule comprises the amino acid sequence of SEQ ID NO: 41. In an embodiment, the GM-CSF molecule comprises the amino acid sequence of SEQ ID NO: 41 without the leader sequence. In an embodiment, the GM-CSF molecule comprises the amino acid sequence of SEQ ID NO: 43. In an embodiment, the GM-CSF molecule comprises the amino acid sequence of SEQ ID NO: 43 without the leader sequence.

In an embodiment, the polynucleotide, e.g., second polynucleotide (e.g., mRNA) encoding the GM-CSF molecule comprises a nucleotide sequence having at least 85%, 90%, 95%, 96%, 97%, 98%, 99% or 100% identity to the nucleic acid sequence of SEQ ID NO: 15. In an embodiment, the polynucleotide, e.g., second polynucleotide encoding the GM-CSF molecule comprises the nucleotide sequence of SEQ ID NO: 15. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 14.

In an embodiment, the polynucleotide, e.g., second polynucleotide (e.g., mRNA) encoding the GM-CSF molecule comprises a nucleotide sequence having at least 85%, 90%, 95%, 96%, 97%, 98%, 99% or 100% identity to the nucleic acid sequence of SEQ ID NO: 38. In an embodiment, the polynucleotide, e.g., second polynucleotide encoding the GM-CSF molecule comprises the nucleotide sequence of SEQ ID NO: 38. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 188.

In an embodiment, the polynucleotide, e.g., second polynucleotide (e.g., mRNA) encoding the GM-CSF molecule comprises a nucleotide sequence having at least 85%, 90%, 95%, 96%, 97%, 98%, 99% or 100% identity to the nucleic acid sequence of SEQ ID NO: 40. In an embodiment, the polynucleotide, e.g., second polynucleotide encoding the GM-CSF molecule comprises the nucleotide sequence of SEQ ID NO: 40. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 39.

In an embodiment, the polynucleotide, e.g., second polynucleotide (e.g., mRNA) encoding the GM-CSF molecule comprises a nucleotide sequence having at least 85%, 90%, 95%, 96%, 97%, 98%, 99% or 100% identity to the nucleic acid sequence of SEQ ID NO: 42. In an embodiment, the polynucleotide, e.g., second polynucleotide encoding the GM-CSF molecule comprises the nucleotide sequence of SEQ ID NO: 42. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 41.

In an embodiment, the polynucleotide, e.g., second polynucleotide (e.g., mRNA) encoding the GM-CSF molecule comprises a nucleotide sequence having at least 85%, 90%, 95%, 96%, 97%, 98%, 99% or 100% identity to the nucleic acid sequence of SEQ ID NO: 44. In an embodiment, the polynucleotide, e.g., second polynucleotide encoding the GM-CSF molecule comprises the nucleotide sequence of SEQ ID NO: 44. In an embodiment, the polynucleotide, e.g., second polynucleotide encodes a GM-CSF molecule having 85%, 90%, 95%, 96%, 97%, 98%, 99% or 100% identity to the amino acid sequence of SEQ ID NO: 43.

In an aspect, an LNP composition disclosed herein comprises a polynucleotide encoding a GM-CSF molecule. In an embodiment, the GM-CSF molecule further comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the half-life extender is human serum albumin (HSA). In an embodiment, the half-life extender is mouse serum albumin (MSA). In an embodiment, the half-life extender is cyno serum albumin (CSA). In an embodiment, the half-life extender is rat serum albumin (RSA).

In an embodiment, the half-life extender is human serum albumin (HSA). In an embodiment, HSA comprises an amino acid sequence having at least 85%, 90%, 95%, 96%, 97%, 98%, 99% or 100% identity to the amino acid sequence of SEQ ID NO: 8. In an embodiment, HSA comprises the amino acid sequence of SEQ ID NO: 8.

In an embodiment, the LNP comprises a polynucleotide encoding a GM-CSF molecule comprising a half-life extender. In an embodiment, the half-life extender is human serum albumin (HSA). In an embodiment, the GM-CSF molecule comprising HSA, e.g., HSA-GM-CSF, comprises an amino acid sequence having at least 85%, 90%, 95%, 96%, 97%, 98%, 99% or 100% identity to an HSA-GM-CSF sequence provided in Table 3A or 3B. In an embodiment, the GM-CSF molecule comprising HSA, e.g., HSA-GM-CSF, comprises the amino acid sequence of an HSA-GM-CSF sequence provided in Table 3A or 3B. In an embodiment, the half-life extender is human serum albumin (HSA). In an embodiment, the GM-CSF molecule comprising HSA, e.g., HSA-GM-CSF, comprises an amino acid sequence having at least 85%, 90%, 95%, 96%, 97%, 98%, 99% or 100% identity to SEQ ID NO: 16. In an embodiment, the GM-CSF molecule comprising HSA, e.g., HSA-GM-CSF, comprises the amino acid sequence of SEQ ID NO: 16.

In an embodiment, an LNP composition comprising a second polynucleotide (e.g., mRNA) encoding a GM-CSF molecule comprising a half-life extender comprises a nucleotide sequence having at least 85%, 90%, 95%, 96%, 97%, 98%, 99% or 100% identity to the nucleic acid sequence of SEQ ID NO: 24. In an embodiment, the second polynucleotide encoding the GM-CSF molecule comprising a half-life extender comprises the nucleotide sequence of SEQ ID NO: 24.

In an embodiment, the polynucleotide (e.g., mRNA) encoding the GM-CSF molecule further comprises one or more elements, e.g., a 5′ UTR and/or a 3′ UTR disclosed herein, e.g., in Table 4B. In an embodiment, the 5′ UTR and/or 3′UTR comprise one or more micro RNA (mIR) binding sites, e.g., as disclosed herein. Exemplary 5′ UTRs and 3′ UTRs are disclosed in the section entitled “5′ UTR and 3′UTR” herein.

TABLE 3A
Exemplary GM-CSF sequences

| SEQ | | |
|---|---|---|
| ID | Sequence | |
| NO | information | Sequence |
| 188 | Human | MWLQSLLLLGTVACSISAPARSPSPSTQPWEHVNAIQEARRLL |
| | GMCSF | NLSRDTAAEMNETVEVISEMFDLQEPTCLQTRLELYKQGLRG |
| | polypeptide | SLTKLKGPLTMMASHYKQHCPPTPETSCATQIITFESFKENLKD |
| | (leader | FLLVIPFDCWEPVQE |
| | underlined) | |
| 38 | Human | AUGUGGCUGCAGAGCCUGCUGCUCUUGGGCACUGUGGCC |
| | GMCSF | UGCAGCAUCUCUGCACCCGCCCGCUCGCCCAGCCCCAGCA |
| | mRNA | CGCAGCCCUGGGAGCAUGUGAAUGCCAUCCAGGAGGCCC |
| | sequence | GGCGUCUCCUGAACCUGAGUAGAGACACUGCUGCUGAGA |
| | | UGAAUGAAACAGUAGAAGUCAUCUCAGAAAUGUUUGACC |
| | | UCCAGGAGCCGACCUGCCUACAGACCCGCCUGGAGCUGUA |
| | | CAAGCAGGGCCUGCGGGGCAGCCUCACCAAGCUCAAGGGC |
| | | CCCUUGACCAUGAUGGCCAGCCACUACAAGCAGCACUGCC |
| | | CUCCAACCCCGGAAACUUCCUGUGCAACCCAGAUUAUCAC |
| | | CUUUGAAAGUUUCAAAGAGAACCUGAAGGACUUUCUGCU |
| | | UGUCAUCCCCUUUGACUGCUGGGAGCCAGUCCAGGAG |
| 14 | Human | APARSPSPSTQPWEHVNAIQEARRLLNLSRDTAAEMNETVEVI |
| | GMCSF | SEMFDLQEPTCLQTRLELYKQGLRGSLTKLKGPLTMMASHYK |
| | | QHCPPTPETSCATQIITFESFKENLKDFLLVIPFDCWEPVQE |
| 15 | Human | AGUACACAGA GAGAAAGGCU AAAGUUCUCU |
| | GMCSF | GGAGGAUGUG GCUGCAGAGC CUGCUGCUCU |
| | mRNA | UGGGCACUGU GGCCUGCAGC AUCUCUGCAC |
| | sequence-2 | CCGCCCGCUC GCCCAGCCCC AGCACGCAGC |
| | | CCUGGGAGCA UGUGAAUGCC AUCCAGGAGG |
| | | CCCGGCGUCU CCUGAACCUG AGUAGAGACA |
| | | CUGCUGCUGA GAUGAAUGAA ACAGUAGAAG |
| | | UCAUCUCAGA AAUGUUUGAC CUCCAGGAGC |
| | | CGACCUGCCU ACAGACCCGC CUGGAGCUGU |
| | | ACAAGCAGGG CCUGCGGGGC AGCCUCACCA |
| | | AGCUCAAGGG CCCCUUGACC AUGAUGGCCA |
| | | GCCACUACAA GCAGCACUGC CCUCCAACCC |
| | | CGGAAACUUC CUGUGCAACC CAGAUUAUCA |
| | | CCUUUGAAAG UUUCAAAGAG AACCUGAAGG |
| | | ACUUUCUGCU UGUCAUCCCC UUUGACUGCU |
| | | GGGAGCCAGU CCAGGAGUGA GACCGGCCAG |
| | | AUGAGGCUGG CCAAGCCGGG GAGCUGCUCU |
| | | CUCAUGAAAC AAGAGCUAGA AACUCAGGAU |
| | | GGUCAUCUUG GAGGGACCAA GGGGUGGGCC |
| | | ACAGCCAUGG UGGGAGUGGC CUGGACCUGC |
| | | CCUGGGCCAC ACUGACCCUG AUACAGGCAU |
| | | GGCAGAAGAA UGGGAAUAUU UUAUACUGAC |
| | | AGAAAUCAGU AAUAUUUAUA UAUUUAUAUU |
| | | UUUAAAAUAU UUAUUUAUUU AUUUAUUUAA |
| | | GUUCAUAUUC CAUAUUUAUU CAAGAUGUUU |
| | | UACCGUAAUA AUUAUUAUUA AAAAUAUGCU UCUACUUG |
| 39 | Mouse | MWLQNLLFLGIVVYSLSAPTRSPITVTRPWKHVEAIKEALNLL |
| | GMCSF | DDMPVTLNEEVEVVSNEFSFKKLTCVQTRLKIFEQGLRGNFT |
| | polypeptide | KLKGALNMTASYYQTYCPPTPETDCETQVTTYADFIDSLKTFL |
| | (leader | TDIPFECKKPGQK |
| | underlined) | |
| 40 | Mouse | AUGUGGCUUCAGAAUCUCUUGUUUCUUGGAAUCGUCGUGUACAGCCU |
| | GMCSF m | GUCAGCCCCAACUAGAUCGCCUAUCACUGUGACGCGCCCGUGGAAGC |
| | RNA | ACGUGGAAGCCAUCAAGGAGGCUCUGAAUCUGCUCGACGAUAUGCCA |
| | | GUGACCCUGAACGAGGAAGUCGAAGUGGUGUCCAACGAAUUUUCCUU |
| | | CAAGAAGUUGACCUGUGUUCAGACCCGGCUGAAGAUUUUCGAGCAGG |
| | | GCCUCAGGGGAAACUUCACCAAACUGAAGGGUGCACUGAACAUGACC |
| | | GCCAGCUACUACCAGACCUAUUGCCCUCCGACUCCGGAAACUGAUUG |
| | | CGAGACUCAAGUCACCACCUACGCGGACUUCAUCGACUCGCUCAAGA |
| | | CGUUCCUGACUGACAUCCCCUUCGAGUGCAAGAAGCCGGGGCAGAAA |
| 41 | Rat GMCSF | MWLQNLLFLGIVVYSFSAPTRSPNPVTRPWKHVDAIKEALSLLNDMRALE |
| | polypeptide | NEKNEDVDIISNEFSIQRPTCVQTRLKLYKQGLRGNLTKLNGALTMIASHY |
| | (leader | QTNCPPTPETDCEIEVTTFEDFIKNLKGFLFDIPFDCWKPVQK |
| | underlined) | |
| 42 | Rat GMCSF | AUGUGGCUGCAGAACCUGCUGUUCCUGGGCAUCGUGGUGUACAGCUU |
| | mRNA | CAGCGCCCCUACCAGAAGCCCUAACCCUGUGACCAGACCUUGGAAGCA |
| | | CGUGGACGCCAUCAAGGAGGCCCUGAGCCUGCUGAACGACAUGAGAG |
| | | CCCUGGAGAACGAGAAGAACGAGGACGUGGACAUCAUCAGCAACGAG |
| | | UUCAGCAUCCAGAGACCUACCUGCGUGCAGACCAGACUGAAGCUGUA |
| | | CAAGCAGGGCCUGAGAGGCAACCUGACCAAGCUGAACGGCGCCCUGA |
| | | CCAUGAUCGCCAGCCACUACCAGACCAACUGCCCUCCUACCCCUGAGA |
| | | CCGACUGCGAGAUCGAGGUGACCACCUUCGAGGACUUCAUCAAGAAC |
| | | CUGAAGGGCUUCCUGUUCGACAUCCCUUUCGACUGCUGGAAGCCUGU |
| | | GCAGAAG |
| 43 | Cyno GMCSF | MWLQGLLLLGTVACSISAPARSPSPGTQPWEHVNAIQEARRLLNLSRDTAA |
| | polypeptide | EMNKTVEVVSEMFDLQEPSCLQTRLELYKQGLQGSLTKLKGPLTMMASHY |
| | (leader | KQHCPPTPETSCATQIITFQSFKENLKDFLLVIPFDCWEPVQE |
| | underlined) | |
| 44 | Cyno GMCSF | AUGUGGCUGCAGGGCCUGCUGCUGCUGGGCACCGUGGCCUGCAGCAU |
| | mRNA | CAGCGCCCCUGCCAGAAGCCCUAGCCCUGGCACCCAGCCUUGGGAGCA |
| | | CGUGAACGCCAUCCAGGAGGCCAGAAGACUGCUGAACCUGAGCAGAG |
| | | ACACCGCCGCCGAGAUGAACAAGACCGUGGAGGUGGUGAGCGAGAUG |
| | | UUCGACCUGCAGGAGCCUAGCUGCCUGCAGACCAGACUGGAGCUGUA |
| | | CAAGCAGGGCCUGCAGGGCAGCCUGACCAAGCUGAAGGGCCCUCUGA |
| | | CCAUGAUGGCCAGCCACUACAAGCAGCACUGCCCUCCUACCCCUGAGA |
| | | CCAGCUGCGCCACCCAGAUCAUCACCUUCCAGAGCUUCAAGGAGAACC |
| | | UGAAGGACUUCCUGCUGGUGAUCCCUUUCGACUGCUGGGAGCCUGUG |
| | | CAGGAG |
| 16 | NVL(SdP) | MKWVTFISLLFLFSSAYSRGVFRRDAHKSEVAHRFKDLGEE |
| | HSA- | NFKALVLIAFAQYLQQCPFEDHVKLVNEVTEFAKTCVADESA |
| | hsGMCSF | ENCDKSLHTLFGDKLCTVATLRETYGEMADCCAKQEPERNE |
| | (underlined) | CFLQHKDDNPNLPRLVRPEVDVMCTAFHDNEETFLKKYLYEI |
| | Note: leader | ARRHPYFYAPELLFFAKRYKAAFTECCQAADKAACLLPKLDE |
| | sequence in | LRDEGKASSAKQRLKCASLQKFGERAFKAWAVARLSQRFPK |
| | bold and | AEFAEVSKLVTDLTKVHTECCHGDLLECADDRADLAKYICEN |
| | underline; | QDSISSKLKECCEKPLLEKSHCIAEVENDEMPADLPSLAADFV |
| | linker | ESKDVCKNYAEAKDVFLGMFLYEYARRHPDYSVVLLLRLAK |
| | italicized and | TYETTLEKCCAAADPHECYAKVFDEFKPLVEEPQNLIKQNCE |
| | underlined | LFEQLGEYKFQNALLVRYTKKVPQVSTPTLVEVSRNLGKVGS |
| | | KCCKHPEAKRMPCAEDYLSVVLNQLCVLHEKTPVSDRVTKC |
| | | CTESLVNRRPCFSALEVDETYVPKEFNAETFTFHADICTLSEKE |
| | | RQIKKQTALVELVKHKPKATKEQLKAVMDDFAAFVEKCCKA |
| | | DDKETCFAEEGKKLVAASQAALGLGGGSAPARSPSPSTQPWE |
| | | HVNAIQEARRLLNLSRDTAAEMNETVEVISEMFDLQEPTCLQ |
| | | TRLELYKQGLRGSLTKLKGPLTMMASHYKQHCPPTPETSCAT |
| | | QIITFESFKENLKDFLLVIPFDCWEPVQE |
| 24 | NVL(SdP) | AUGAAGUGGGUGACCUUCAUCAGCCUGCUGUUCCUGUUCAGCAGCGC |
| | hsGMCSF | CUGAUCGCCUUCGCCCAGUACCUGCAGCAGUGCCCUUUCGAGGACCAC |
| | HSA- | CUACAGCAGAGGCGUGUUCAGAAGAGACGCCCACAAGAGCGAGGUGG |
| | | CCCACAGAUUCAAGGACCUGGGCGAGGAGAACUUCAAGGCCCUGGUG |
| | | GUGAAGCUGGUGAACGAGGUGACCGAGUUCGCCAAGACCUGCGUGGC |
| | | CGACGAGAGCGCCGAGAACUGCGACAAGAGCCUGCACACCCUGUUCG |
| | | GCGACAAGCUGUGCACCGUGGCCACCCUGAGAGAAACUUACGGCGAG |
| | | AUGGCCGACUGCUGCGCCAAGCAGGAGCCAGAGCGGAACGAGUGCUU |
| | | CCUGCAACACAAGGACGACAACCCUAACCUGCCUAGACUGGUCCGGCC |
| | | UGAGGUGGACGUGAUGUGCACGGCCUUCCACGACAACGAGGAGACUU |
| | | UCCUGAAGAAGUACCUGUACGAGAUCGCCAGAAGACACCCUUACUUC |
| | | UACGCCCCUGAGCUCCUGUUCUUCGCGAAGAGAUACAAGGCCGCCUU |
| | | CACCGAGUGCUGCCAGGCCGCCGACAAGGCAGCUUGCCUGCUGCCUA |
| | | AGCUGGACGAGCUGAGAGACGAGGGCAAGGCCUCCUCAGCUAAGCAG |
| | | AGACUGAAGUGCGCCAGCCUGCAGAAGUUCGGUGAGAGAGCAUUCAA |
| | | GGCUUGGGCCGUCGCAAGACUGUCACAGAGAUUCCCUAAGGCAGAAU |
| | | UCGCGGAGGUGAGCAAGCUAGUGACCGACCUGACCAAGGUGCAUACA |
| | | GAGUGCUGCCACGGCGACCUGCUGGAGUGCGCCGACGACAGAGCCGA |
| | | CCUGGCCAAGUACAUCUGCGAGAACCAGGACAGCAUCAGCUCCAAGC |
| | | UGAAGGAGUGCUGUGAGAAGCCUCUGCUGGAGAAGUCACACUGCAUU |
| | | GCCGAGGUCGAGAACGACGAGAUGCCUGCCGAUCUUCCUAGCCUUGC |
| | | CGCCGAUUUCGUGGAGAGCAAGGACGUGUGCAAGAACUACGCCGAGG |
| | | CAAAGGACGUGUUCCUGGGCAUGUUCCUUUACGAAUACGCUCGCCGG |
| | | CAUCCAGACUACAGCGUGGUGCUGCUGCUGAGAUUGGCCAAGACUUA |
| | | CGAGACGACCCUCGAGAAGUGUUGCGCAGCAGCUGAUCCUCACGAGU |
| | | GUUACGCCAAGGUGUUCGACGAGUUCAAGCCGCUUGUGGAGGAGCCU |
| | | CAGAACCUGAUCAAGCAGAAUUGUGAGCUGUUCGAGCAGCUGGGUGA |
| | | GUACAAGUUCCAGAACGCCCUGCUGGUGCGCUACACCAAGAAGGUGC |
| | | CUCAAGUGUCUACCCCUACCCUGGUUGAAGUUUCCCGCAACCUGGGC |
| | | AAGGUGGGCAGCAAGUGCUGCAAGCAUCCUGAAGCAAAGAGGAUGCC |
| | | UUGCGCCGAGGACUACCUGUCAGUGGUCCUUAACCAGCUGUGCGUGC |
| | | UGCACGAGAAGACCCCUGUGAGCGACAGAGUGACAAAGUGUUGUACC |
| | | GAGAGCCUGGUCAACAGAAGACCUUGCUUCAGCGCCCUGGAAGUCGA |
| | | CGAGACAUACGUGCCUAAGGAGUUCAACGCCGAAACCUUCACCUUCC |
| | | ACGCCGACAUCUGCACACUGAGCGAGAAGGAGAGACAGAUCAAGAAG |
| | | CAGACCGCCCUGGUCGAGUUGGUGAAGCACAAGCCUAAGGCCACCAA |
| | | GGAGCAACUCAAGGCCGUGAUGGACGACUUCGCGGCCUUCGUUGAGA |
| | | AGUGCUGUAAGGCUGACGACAAGGAGACGUGCUUCGCUGAGGAGGGU |
| | | AAGAAGCUUGUCGCCGCCUCUCAGGCCGCUUUGGGACUCGGCGGCGG |
| | | CAGUGCGCCUGCCAGAAGCCCUUCCCCAUCUACCCAGCCUUGGGAGCA |
| | | CGUGAACGCCAUCCAGGAGGCCAGACGUCUGCUGAACCUGUCACGGG |
| | | AUACCGCAGCUGAGAUGAACGAAACUGUUGAGGUCAUCAGCGAGAUG |
| | | UUCGACCUACAGGAACCUACCUGCUUGCAGACCAGACUGGAGCUGUA |
| | | CAAGCAGGGAUUAAGAGGCUCCCUGACGAAGCUUAAGGGCCCUCUGA |
| | | CCAUGAUGGCCAGCCACUAUAAGCAGCACUGCCCUCCUACCCCUGAAA |
| | | CGUCGUGUGCUACCCAGAUCAUCACCUUCGAGAGCUUCAAGGAGAAU |
| | | CUGAAGGACUUCCUGCUCGUUAUUCCGUUCGAUUGUUGGGAGCCUGU |
| | | GCAGGAG |

Without wishing to be bound by theory, a skilled person would understand that in some embodiments the amino acid sequence of RGVFRRD can constitute part of the leader sequence described herein as HSA is generally made as a pre-pro-peptide.

In some embodiments, a polynucleotide of the present disclosure, for example a polynucleotide comprising an mRNA nucleotide sequence encoding a polypeptide, comprises (1) a 5′ cap, e.g., as disclosed herein, (2) a 5′ UTR, e.g., as provided in Table 3B, (3) a nucleotide sequence ORF provided in Table 3A, or 3B, (4) a stop codon, (5) a 3′UTR, e.g., as provided in Table 3B, and (6) a poly-A tail, e.g., as disclosed herein, e.g., a poly-A tail of about 100 residues, e.g., SEQ ID NO: 29.

In some embodiments, a polynucleotide comprising an mRNA nucleotide sequence encoding a GM-CSF polypeptide, comprises SEQ ID NO: 204 that consists from 5′ to 3′ end: 5′ UTR of SEQ ID NO: 202, ORF sequence of SEQ ID NO: 201, 3′ UTR of SEQ ID NO: 203 and Poly A tail of SEQ ID NO: 29.

In some embodiments, a polynucleotide comprising an mRNA nucleotide sequence encoding a GM-CSF polypeptide, comprises SEQ ID NO: 209 that consists from 5′ to 3′ end: 5′ UTR of SEQ ID NO: 207, ORF sequence of SEQ ID NO: 206, 3′ UTR of SEQ ID NO: 208 and Poly A tail of SEQ ID NO: 29.

In some embodiments, a polynucleotide comprising an mRNA nucleotide sequence encoding a GM-CSF polypeptide, comprises SEQ ID NO: 214 that consists from 5′ to 3′ end: 5′ UTR of SEQ ID NO: 212, ORF sequence of SEQ ID NO: 211, 3′ UTR of SEQ ID NO: 213 and Poly A tail of SEQ ID NO: 29.

In some embodiments, a polynucleotide comprising an mRNA nucleotide sequence encoding a GM-CSF polypeptide, comprises SEQ ID NO: 219 that consists from 5′ to 3′ end: 5′ UTR of SEQ ID NO: 217, ORF sequence of SEQ ID NO: 216, 3′ UTR of SEQ ID NO: 218 and Poly A tail of SEQ ID NO: 29.

In some embodiments, a polynucleotide comprising an mRNA nucleotide sequence encoding a GM-CSF polypeptide, comprises SEQ ID NO: 224 that consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 29.

TABLE 3B
Exemplary GM-CSF construct sequences
Note: “G5” indicates that all uracils (U) in the mRNA are replaced by N1-methylpseudouracils.

| | mRNA | ORF Sequence | ORF Sequence | 5′ UTR | 3′ UTR | Construct |
|---|---|---|---|---|---|---|
| | Name | (Amino Acid) | (Nucleotide) | Sequence | Sequence | Sequence |
| SEQ | | | | | | |
| ID | | | | | | |
| NO: | | 200 | 201 | 202 | 203 | 204 |
| | Cyno.GMC | MWLQGLLLLGTV | AUGUGGCUGCA | GGGAAA | UGAUAA | SEQ ID |
| | SF | ACSISAPARSPSPG | GGGCCUGCUGC | UAAGAG | UAGGCU | NO: 204 |
| | G5 | TQPWEHVNAIQEA | UGCUGGGCACC | AGAAAA | GGAGCC | consists |
| | Cap: C1 | RRLLNLSRDTAAE | GUGGCCUGCAG | GAAGAG | UCGGUG | from 5′ to |
| | Poly A | MNKTVEVVSEMF | CAUCAGCGCCCC | UAAGAA | GCCUAG | 3′ end: 5′ |
| | tail: 100 nt | DLQEPSCLQTRLE | UGCCAGAAGCCC | GAAAUA | CUUCUU | UTR of |
| | | LYKQGLQGSLTKL | UAGCCCUGGCAC | UAAGAC | GCCCCU | SEQ ID |
| | | KGPLTMMASHYK | CCAGCCUUGGG | CCCGGC | UGGGCC | NO: 202, |
| | | QHCPPTPETSCAT | AGCACGUGAAC | GCCGCC | UCCCCCC | ORF |
| | | QIITFQSFKENLKD | GCCAUCCAGGA | ACC | AGCCCC | sequence |
| | | FLLVIPFDCWEPV | GGCCAGAAGAC | | UCCUCC | of SEQ ID |
| | | QE | UGCUGAACCUG | | CCUUCC | NO: 201, |
| | | | AGCAGAGACAC | | UGCACC | 3′ UTR of |
| | | | CGCCGCCGAGAU | | CGUACC | SEQ ID |
| | | | GAACAAGACCG | | CCCGUG | NO: 203 |
| | | | UGGAGGUGGUG | | GUCUUU | and Poly |
| | | | AGCGAGAUGUU | | GAAUAA | A tail of |
| | | | CGACCUGCAGG | | AGUCUG | SEQ ID |
| | | | AGCCUAGCUGCC | | AGUGGG | NO: 29 |
| | | | UGCAGACCAGA | | CGGC | |
| | | | CUGGAGCUGUA | | | |
| | | | CAAGCAGGGCC | | | |
| | | | UGCAGGGCAGC | | | |
| | | | CUGACCAAGCU | | | |
| | | | GAAGGGCCCUC | | | |
| | | | UGACCAUGAUG | | | |
| | | | GCCAGCCACUAC | | | |
| | | | AAGCAGCACUG | | | |
| | | | CCCUCCUACCCC | | | |
| | | | UGAGACCAGCU | | | |
| | | | GCGCCACCCAGA | | | |
| | | | UCAUCACCUUCC | | | |
| | | | AGAGCUUCAAG | | | |
| | | | GAGAACCUGAA | | | |
| | | | GGACUUCCUGC | | | |
| | | | UGGUGAUCCCU | | | |
| | | | UUCGACUGCUG | | | |
| | | | GGAGCCUGUGC | | | |
| | | | AGGAG | | | |
| SEQ | | | | | | |
| ID | | | | | | |
| NO: | | 205 | 206 | 207 | 208 | 209 |
| | Rn.GMCSF | MWLQNLLFLGIVV | AUGUGGCUGCA | GGGAAA | UGAUAA | SEQ ID |
| | G5 | YSFSAPTRSPNPVT | GAACCUGCUGU | UAAGAG | UAGGCU | NO: 209 |
| | Cap: C1 | RPWKHVDAIKEAL | UCCUGGGCAUC | AGAAAA | GGAGCC | consists |
| | Poly A | SLLNDMRALENEK | GUGGUGUACAG | GAAGAG | UCGGUG | from 5′ to |
| | tail: 100 nt | NEDVDIISNEFSIQ | CUUCAGCGCCCC | UAAGAA | GCCUAG | 3′ end: 5′ |
| | | RPTCVQTRLKLYK | UACCAGAAGCCC | GAAAUA | CUUCUU | UTR of |
| | | QGLRGNLTKLNG | UAACCCUGUGA | UAAGAC | GCCCCU | SEQ ID |
| | | ALTMIASHYQTNC | CCAGACCUUGG | CCCGGC | UGGGCC | NO: 207, |
| | | PPTPETDCEIEVTT | AAGCACGUGGA | GCCGCC | UCCCCCC | ORF |
| | | FEDFIKNLKGFLFD | CGCCAUCAAGG | ACC | AGCCCC | sequence |
| | | IPFDCWKPVQK | AGGCCCUGAGCC | | UCCUCC | of SEQ ID |
| | | | UGCUGAACGAC | | CCUUCC | NO: 206, |
| | | | AUGAGAGCCCU | | UGCACC | 3′ UTR of |
| | | | GGAGAACGAGA | | CGUACC | SEQ ID |
| | | | AGAACGAGGAC | | CCCGUG | NO: 208 |
| | | | GUGGACAUCAU | | GUCUUU | and Poly |
| | | | CAGCAACGAGU | | GAAUAA | A tail of |
| | | | UCAGCAUCCAG | | AGUCUG | SEQ ID |
| | | | AGACCUACCUGC | | AGUGGG | NO: 29 |
| | | | GUGCAGACCAG | | CGGC | |
| | | | ACUGAAGCUGU | | | |
| | | | ACAAGCAGGGC | | | |
| | | | CUGAGAGGCAA | | | |
| | | | CCUGACCAAGCU | | | |
| | | | GAACGGCGCCCU | | | |
| | | | GACCAUGAUCG | | | |
| | | | CCAGCCACUACC | | | |
| | | | AGACCAACUGCC | | | |
| | | | CUCCUACCCCUG | | | |
| | | | AGACCGACUGC | | | |
| | | | GAGAUCGAGGU | | | |
| | | | GACCACCUUCGA | | | |
| | | | GGACUUCAUCA | | | |
| | | | AGAACCUGAAG | | | |
| | | | GGCUUCCUGUU | | | |
| | | | CGACAUCCCUUU | | | |
| | | | CGACUGCUGGA | | | |
| | | | AGCCUGUGCAG | | | |
| | | | AAG | | | |
| SEQ | | | | | | |
| ID | | | | | | |
| NO: | | 210 | 211 | 212 | 213 | 214 |
| | Mm.GMCSF | MWLQNLLFLGIVV | AUGUGGCUUCA | GGGAAA | GGGAAA | SEQ ID |
| | G5 | YSLSAPTRSPITVT | GAAUCUCUUGU | UAAGAG | UAAGAG | NO: 214 |
| | Cap: C1 | RPWKHVEAIKEAL | UUCUUGGAAUC | AGAAAA | AGAAAA | consists |
| | Poly A | NLLDDMPVTLNEE | GUCGUGUACAG | GAAGAG | GAAGAG | from 5′ to |
| | tail: 100 nt | VEVVSNEFSFKKL | CUUAUCAGCCCC | UAAGAA | UAAGAA | 3′ end: 5′ |
| | | TCVQTRLKIFEQG | AACUAGAUCGC | GAAAUA | GAAAUA | UTR of |
| | | LRGNFTKLKGALN | CUAUCACGGUG | UAAGAG | UAAGAG | SEQ ID |
| | | MTASYYQTYCPPT | ACGCGCCCGUGG | CCACC | CCACC | NO: 212, |
| | | PETDCETQVTTYA | AAGCACGUAGA | | | ORF |
| | | DFIDSLKTFLTDIPF | AGCCAUCAAGG | | | sequence |
| | | ECKKPGQK | AGGCUCUCAAU | | | of SEQ ID |
| | | | UUACUCGACGA | | | NO: 211, |
| | | | UAUGCCAGUGA | | | 3′ UTR of |
| | | | CCCUUAACGAG | | | SEQ ID |
| | | | GAAGUCGAAGU | | | NO: 213 |
| | | | GGUGUCCAACG | | | and Poly |
| | | | AAUUUUCCUUC | | | A tail of |
| | | | AAGAAGUUGAC | | | SEQ ID |
| | | | CUGUGUUCAGA | | | NO: 29 |
| | | | CCCGGCUGAAG | | | |
| | | | AUUUUCGAGCA | | | |
| | | | GGGCCUCAGGG | | | |
| | | | GAAACUUCACC | | | |
| | | | AAACUGAAGGG | | | |
| | | | UGCACUGAACA | | | |
| | | | UGACCGCCAGCU | | | |
| | | | ACUACCAGACCU | | | |
| | | | AUUGCCCUCCGA | | | |
| | | | CUCCGGAAACU | | | |
| | | | GAUUGCGAGAC | | | |
| | | | UCAAGUCACCAC | | | |
| | | | CUACGCGGACU | | | |
| | | | UCAUCGACUCGC | | | |
| | | | UCAAGACGUUC | | | |
| | | | CUGACUGACAU | | | |
| | | | CCCCUUCGAGUG | | | |
| | | | CAAGAAGCCGG | | | |
| | | | GGCAGAAA | | | |
| SEQ | | | | | | |
| ID | | | | | | |
| NO: | | 215 | 216 | 217 | 218 | 219 |
| | hs.GMCSF | MWLQSLLLLGTV | AUGUGGCUGCA | GGGAAA | UGAUAA | SEQ ID |
| | G5 | ACSISAPARSPSPS | GAGCCUGCUGC | UAAGAG | UAGGCU | NO: 219 |
| | Cap: C1 | TQPWEHVNAIQEA | UCUUGGGCACU | AGAAAA | GGAGCC | consists |
| | Poly A | RRLLNLSRDTAAE | GUGGCCUGCAG | GAAGAG | UCGGUG | from 5′ to |
| | tail: 100 nt | MNETVEVISEMFD | CAUCUCUGCACC | UAAGAA | GCCUAG | 3′ end: 5′ |
| | | LQEPTCLQTRLEL | CGCCCGCUCGCC | GAAAUA | CUUCUU | UTR of |
| | | YKQGLRGSLTKLK | CAGCCCCAGCAC | UAAGAC | GCCCCU | SEQ ID |
| | | GPLTMMASHYKQ | GCAGCCCUGGG | CCCGGC | UGGGCC | NO: 217, |
| | | HCPPTPETSCATQII | AGCAUGUGAAU | GCCGCC | UCCCCCC | ORF |
| | | TFESFKENLKDFLL | GCCAUCCAGGA | ACC | AGCCCC | sequence |
| | | VIPFDCWEPVQE | GGCCCGGCGUCU | | UCCUCC | of SEQ ID |
| | | | CCUGAACCUGA | | CCUUCC | NO: 216, |
| | | | GUAGAGACACU | | UGCACC | 3′ UTR of |
| | | | GCUGCUGAGAU | | CGUACC | SEQ ID |
| | | | GAAUGAAACAG | | CCCGUG | NO: 218 |
| | | | UAGAAGUCAUC | | GUCUUU | and Poly |
| | | | UCAGAAAUGUU | | GAAUAA | A tail of |
| | | | UGACCUCCAGG | | AGUCUG | SEQ ID |
| | | | AGCCGACCUGCC | | AGUGGG | NO: 29 |
| | | | UACAGACCCGCC | | CGGC | |
| | | | UGGAGCUGUAC | | | |
| | | | AAGCAGGGCCU | | | |
| | | | GCGGGGCAGCC | | | |
| | | | UCACCAAGCUCA | | | |
| | | | AGGGCCCCUUG | | | |
| | | | ACCAUGAUGGC | | | |
| | | | CAGCCACUACAA | | | |
| | | | GCAGCACUGCCC | | | |
| | | | UCCAACCCCGGA | | | |
| | | | AACUUCCUGUG | | | |
| | | | CAACCCAGAUU | | | |
| | | | AUCACCUUUGA | | | |
| | | | AAGUUUCAAAG | | | |
| | | | AGAACCUGAAG | | | |
| | | | GACUUUCUGCU | | | |
| | | | UGUCAUCCCCUU | | | |
| | | | UGACUGCUGGG | | | |
| | | | AGCCAGUCCAG | | | |
| | | | GAG | | | |
| SEQ | | | | | | |
| ID | | | | | | |
| NO: | | 220 | 221 | 222 | 223 | 224 |
| | NVL(SdP)_ | MKWVTFISLLFLFS | AUGAAGUGGGU | GGGAAA | UGAUAA | SEQ ID |
| | HSA- | SAYSRGVFRRDAH | GACCUUCAUCA | UAAGAG | UAGGCU | NO: 224 |
| | hsGMCSF | KSEVAHRFKDLGE | GCCUGCUGUUCC | AGAAAA | GGAGCC | consists |
| | G5 | ENFKALVLIAFAQ | UGUUCAGCAGC | GAAGAG | UCGGUG | from 5′ to |
| | Cap: C1 | YLQQCPFEDHVKL | GCCUACAGCAG | UAAGAA | GCCUAG | 3′ end: 5′ |
| | Poly A | VNEVTEFAKTCVA | AGGCGUGUUCA | GAAAUA | CUUCUU | UTR of |
| | tail: 100 nt | DESAENCDKSLHT | GAAGAGACGCC | UAAGAC | GCCCCU | SEQ ID |
| | | LFGDKLCTVATLR | CACAAGAGCGA | CCCGGC | UGGGCC | NO: 222, |
| | | ETYGEMADCCAK | GGUGGCCCACA | GCCGCC | UCCCCCC | ORF |
| | | QEPERNECFLQHK | GAUUCAAGGAC | ACC | AGCCCC | sequence |
| | | DDNPNLPRLVRPE | CUGGGCGAGGA | | UCCUCC | of SEQ ID |
| | | VDVMCTAFHDNE | GAACUUCAAGG | | CCUUCC | NO: 221, |
| | | ETFLKKYLYEIAR | CCCUGGUGCUG | | UGCACC | 3′ UTR of |
| | | RHPYFYAPELLFF | AUCGCCUUCGCC | | CGUACC | SEQ ID |
| | | AKRYKAAFTECCQ | CAGUACCUGCA | | CCCGUG | NO: 223 |
| | | AADKAACLLPKLD | GCAGUGCCCUU | | GUCUUU | and Poly |
| | | ELRDEGKASSAKQ | UCGAGGACCAC | | GAAUAA | A tail of |
| | | RLKCASLQKFGER | GUGAAGCUGGU | | AGUCUG | SEQ ID |
| | | AFKAWAVARLSQ | GAACGAGGUGA | | AGUGGG | NO: 29 |
| | | RFPKAEFAEVSKL | CCGAGUUCGCCA | | CGGC | |
| | | VTDLTKVHTECCH | AGACCUGCGUG | | | |
| | | GDLLECADDRADL | GCCGACGAGAG | | | |
| | | AKYICENQDSISSK | CGCCGAGAACU | | | |
| | | LKECCEKPLLEKS | GCGACAAGAGC | | | |
| | | HCIAEVENDEMPA | CUGCACACCCUG | | | |
| | | DLPSLAADFVESK | UUCGGCGACAA | | | |
| | | DVCKNYAEAKDV | GCUGUGCACCG | | | |
| | | FLGMFLYEYARRH | UGGCCACCCUGA | | | |
| | | PDYSVVLLLRLAK | GAGAAACUUAC | | | |
| | | TYETTLEKCCAAA | GGCGAGAUGGC | | | |
| | | DPHECYAKVFDEF | CGACUGCUGCGC | | | |
| | | KPLVEEPQNLIKQ | CAAGCAGGAGC | | | |
| | | NCELFEQLGEYKF | CAGAGCGGAAC | | | |
| | | QNALLVRYTKKVP | GAGUGCUUCCU | | | |
| | | QVSTPTLVEVSRN | GCAACACAAGG | | | |
| | | LGKVGSKCCKHPE | ACGACAACCCUA | | | |
| | | AKRMPCAEDYLS | ACCUGCCUAGAC | | | |
| | | VVLNQLCVLHEKT | UGGUCCGGCCU | | | |
| | | PVSDRVTKCCTES | GAGGUGGACGU | | | |
| | | LVNRRPCFSALEV | GAUGUGCACGG | | | |
| | | DETYVPKEFNAET | CCUUCCACGACA | | | |
| | | FTFHADICTLSEKE | ACGAGGAGACU | | | |
| | | RQIKKQTALVELV | UUCCUGAAGAA | | | |
| | | KHKPKATKEQLK | GUACCUGUACG | | | |
| | | AVMDDFAAFVEK | AGAUCGCCAGA | | | |
| | | CCKADDKETCFAE | AGACACCCUUAC | | | |
| | | EGKKLVAASQAA | UUCUACGCCCCU | | | |
| | | LGLGGGSAPARSP | GAGCUCCUGUU | | | |
| | | SPSTQPWEHVNAI | CUUCGCGAAGA | | | |
| | | QEARRLLNLSRDT | GAUACAAGGCC | | | |
| | | AAEMNETVEVISE | GCCUUCACCGAG | | | |
| | | MFDLQEPTCLQTR | UGCUGCCAGGCC | | | |
| | | LELYKQGLRGSLT | GCCGACAAGGC | | | |
| | | KLKGPLTMMASH | AGCUUGCCUGC | | | |
| | | YKQHCPPTPETSC | UGCCUAAGCUG | | | |
| | | ATQIITFESFKENL | GACGAGCUGAG | | | |
| | | KDFLLVIPFDCWE | AGACGAGGGCA | | | |
| | | PVQE | AGGCCUCCUCAG | | | |
| | | | CUAAGCAGAGA | | | |
| | | | CUGAAGUGCGC | | | |
| | | | CAGCCUGCAGA | | | |
| | | | AGUUCGGUGAG | | | |
| | | | AGAGCAUUCAA | | | |
| | | | GGCUUGGGCCG | | | |
| | | | UCGCAAGACUG | | | |
| | | | UCACAGAGAUU | | | |
| | | | CCCUAAGGCAG | | | |
| | | | AAUUCGCGGAG | | | |
| | | | GUGAGCAAGCU | | | |
| | | | AGUGACCGACC | | | |
| | | | UGACCAAGGUG | | | |
| | | | CAUACAGAGUG | | | |
| | | | CUGCCACGGCGA | | | |
| | | | CCUGCUGGAGU | | | |
| | | | GCGCCGACGACA | | | |
| | | | GAGCCGACCUG | | | |
| | | | GCCAAGUACAU | | | |
| | | | CUGCGAGAACC | | | |
| | | | AGGACAGCAUC | | | |
| | | | AGCUCCAAGCU | | | |
| | | | GAAGGAGUGCU | | | |
| | | | GUGAGAAGCCU | | | |
| | | | CUGCUGGAGAA | | | |
| | | | GUCACACUGCA | | | |
| | | | UUGCCGAGGUC | | | |
| | | | GAGAACGACGA | | | |
| | | | GAUGCCUGCCG | | | |
| | | | AUCUUCCUAGCC | | | |
| | | | UUGCCGCCGAU | | | |
| | | | UUCGUGGAGAG | | | |
| | | | CAAGGACGUGU | | | |
| | | | GCAAGAACUAC | | | |
| | | | GCCGAGGCAAA | | | |
| | | | GGACGUGUUCC | | | |
| | | | UGGGCAUGUUC | | | |
| | | | CUUUACGAAUA | | | |
| | | | CGCUCGCCGGCA | | | |
| | | | UCCAGACUACA | | | |
| | | | GCGUGGUGCUG | | | |
| | | | CUGCUGAGAUU | | | |
| | | | GGCCAAGACUU | | | |
| | | | ACGAGACGACCC | | | |
| | | | UCGAGAAGUGU | | | |
| | | | UGCGCAGCAGC | | | |
| | | | UGAUCCUCACG | | | |
| | | | AGUGUUACGCC | | | |
| | | | AAGGUGUUCGA | | | |
| | | | CGAGUUCAAGC | | | |
| | | | CGCUUGUGGAG | | | |
| | | | GAGCCUCAGAA | | | |
| | | | CCUGAUCAAGC | | | |
| | | | AGAAUUGUGAG | | | |
| | | | CUGUUCGAGCA | | | |
| | | | GCUGGGUGAGU | | | |
| | | | ACAAGUUCCAG | | | |
| | | | AACGCCCUGCUG | | | |
| | | | GUGCGCUACACC | | | |
| | | | AAGAAGGUGCC | | | |
| | | | UCAAGUGUCUA | | | |
| | | | CCCCUACCCUGG | | | |
| | | | UUGAAGUUUCC | | | |
| | | | CGCAACCUGGGC | | | |
| | | | AAGGUGGGCAG | | | |
| | | | CAAGUGCUGCA | | | |
| | | | AGCAUCCUGAA | | | |
| | | | GCAAAGAGGAU | | | |
| | | | GCCUUGCGCCGA | | | |
| | | | GGACUACCUGU | | | |
| | | | CAGUGGUCCUU | | | |
| | | | AACCAGCUGUG | | | |
| | | | CGUGCUGCACG | | | |
| | | | AGAAGACCCCU | | | |
| | | | GUGAGCGACAG | | | |
| | | | AGUGACAAAGU | | | |
| | | | GUUGUACCGAG | | | |
| | | | AGCCUGGUCAA | | | |
| | | | CAGAAGACCUU | | | |
| | | | GCUUCAGCGCCC | | | |
| | | | UGGAAGUCGAC | | | |
| | | | GAGACAUACGU | | | |
| | | | GCCUAAGGAGU | | | |
| | | | UCAACGCCGAA | | | |
| | | | ACCUUCACCUUC | | | |
| | | | CACGCCGACAUC | | | |
| | | | UGCACACUGAG | | | |
| | | | CGAGAAGGAGA | | | |
| | | | GACAGAUCAAG | | | |
| | | | AAGCAGACCGCC | | | |
| | | | CUGGUCGAGUU | | | |
| | | | GGUGAAGCACA | | | |
| | | | AGCCUAAGGCC | | | |
| | | | ACCAAGGAGCA | | | |
| | | | ACUCAAGGCCG | | | |
| | | | UGAUGGACGAC | | | |
| | | | UUCGCGGCCUUC | | | |
| | | | GUUGAGAAGUG | | | |
| | | | CUGUAAGGCUG | | | |
| | | | ACGACAAGGAG | | | |
| | | | ACGUGCUUCGC | | | |
| | | | UGAGGAGGGUA | | | |
| | | | AGAAGCUUGUC | | | |
| | | | GCCGCCUCUCAG | | | |
| | | | GCCGCUUUGGG | | | |
| | | | ACUCGGCGGCG | | | |
| | | | GCAGUGCGCCU | | | |
| | | | GCCAGAAGCCCU | | | |
| | | | UCCCCAUCUACC | | | |
| | | | CAGCCUUGGGA | | | |
| | | | GCACGUGAACG | | | |
| | | | CCAUCCAGGAG | | | |
| | | | GCCAGACGUCU | | | |
| | | | GCUGAACCUGU | | | |
| | | | CACGGGAUACC | | | |
| | | | GCAGCUGAGAU | | | |
| | | | GAACGAAACUG | | | |
| | | | UUGAGGUCAUC | | | |
| | | | AGCGAGAUGUU | | | |
| | | | CGACCUACAGG | | | |
| | | | AACCUACCUGCU | | | |
| | | | UGCAGACCAGA | | | |
| | | | CUGGAGCUGUA | | | |
| | | | CAAGCAGGGAU | | | |
| | | | UAAGAGGCUCC | | | |
| | | | CUGACGAAGCU | | | |
| | | | UAAGGGCCCUC | | | |
| | | | UGACCAUGAUG | | | |
| | | | GCCAGCCACUAU | | | |
| | | | AAGCAGCACUG | | | |
| | | | CCCUCCUACCCC | | | |
| | | | UGAAACGUCGU | | | |
| | | | GUGCUACCCAG | | | |
| | | | AUCAUCACCUUC | | | |
| | | | GAGAGCUUCAA | | | |
| | | | GGAGAAUCUGA | | | |
| | | | AGGACUUCCUG | | | |
| | | | CUCGUUAUUCC | | | |
| | | | GUUCGAUUGUU | | | |
| | | | GGGAGCCUGUG | | | |
| | | | CAGGAG | | | |

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; a (iv) PEG lipid. These categories of lipids are set forth in more detail below.

### Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

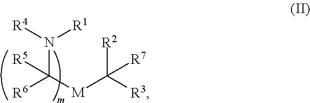

or their N-oxides, or salts or isomers thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected
from —C(O)O—, —OC(O)—, —OC(O)-M″-C(O)O—, —C(O)N(R′)—,
—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.
   Another aspect the disclosure relates to compounds of Formula (III):

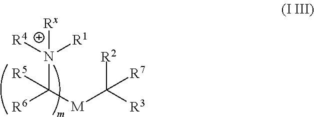

### or its N-oxide,

or a salt or isomer thereof, wherein
   or a salt or isomer thereof, wherein
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆
carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
   R^{x} is selected from the group consisting of C₁₋₆ alkyl, C₂₋₆ alkenyl, —(CH₂)OH, and —(CH₂)ᵥN(R)₂,
   wherein v is selected from 1, 2, 3, 4, 5, and 6;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(OXOR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In certain embodiments, a subset of compounds of Formula (I) includes those of Formula (IA):

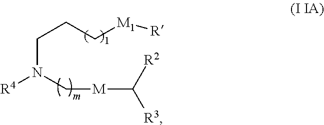

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁-3 alkyl, —(CH₂)ₒC(R¹⁰)₂(CH₂)ₙ₋ₒQ, or —(CH₂)ₒQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(OXOR′)O—, —S—S—, an aryl group, and a heteroaryl group; and R² and R³ are independently selected from the group consisting of H, C₁₋₄ alkyl, and C₂₋₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

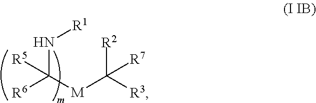

or its N-oxide, or a salt or isomer thereof in which all variables are as defined herein. For example, m is selected from 5, 6, 7, 8, and 9; M and M′ are independently selected
from —C(O)O—, —OC(O)—, —OC(O)-M″-C(O)O—, —C(O)N(R′)—, —P(OXOR′)O—, —S—S—, an aryl group, and a heteroaryl group; and R² and R³ are independently selected from the group consisting of H, C₁₋₄ alkyl, and C₂₋₄ alkenyl. For example, m is 5, 7, or 9. In certain embodiments, a subset of compounds of Formula (I) includes those of Formula (II):

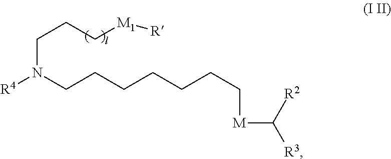

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(OXOR′)O—, —S—S—, an aryl group, and a heteroaryl group; and R² and R³ are independently selected from the group consisting of H, C₁₋₁₄ alkyl, and C₂₋₄ alkenyl.

Another aspect of the disclosure relates to compounds of Formula (I VI):

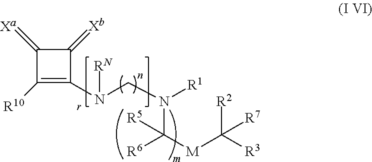

### or its N-oxide,

or a salt or isomer thereof, wherein
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(OXOR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
each R is independently selected from the group consisting of H, C₁₋₃ alkyl, and C₂₋₃ alkenyl; R^{N} is H, or C₁₋₃ alkyl;
each R′ is independently selected from the group consisting of C₁₋₁₈ alkyl, C₂₋₁₈ alkenyl, —R*YR″, —YR″, and H;
each R″ is independently selected from the group consisting of C₃₋₁₈ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I;
X^{a} and X^{b} are each independently O or S;
   R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₕ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;
   m is selected from 5, 6, 7, 8, 9, 10, 11, 12, and 13;
   n is selected from 1, 2, 3, 4, 5, 6, 7, 8, 9, and 10;
   r is 0 or 1;
   t1 is selected from 1, 2, 3, 4, and 5;
   p1 is selected from 1, 2, 3, 4, and 5;
   q1 is selected from 1, 2, 3, 4, and 5; and
   s1 is selected from 1, 2, 3, 4, and 5.

In one embodiment, a subset of compounds of Formula (VI) includes those of Formula (VI-a):

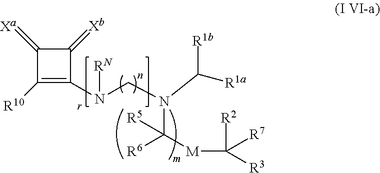

### or its N-oxide,

or a salt or isomer thereof, wherein
   R^{1a} and R^{1b} are independently selected from the group consisting of C₁₋₁₄ alkyl and C₂₋₁₄ alkenyl; and
   R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

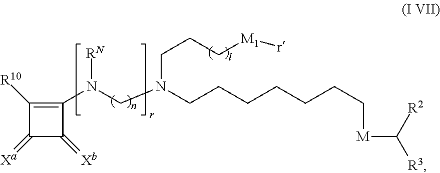

or its N-oxide, or a salt or isomer thereof, wherein
l is selected from 1, 2, 3, 4, and 5;
Mᵢ is a bond or M′; and
R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

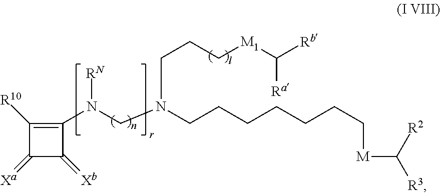

or its N-oxide, or a salt or isomer thereof, wherein
l is selected from 1, 2, 3, 4, and 5;
M₁ is a bond or M′; and
R^{a′} and R^{b′} are independently selected from the group consisting of C₁₋₁₄ alkyl and C₂₋₄ alkenyl; and
   R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₃ alkyl or C₂₋₃ alkenyl. In other embodiments, M″ is C₁i alkyl or C₂₄ alkenyl. In certain embodiments, M″ is C₁₄ alkyl or C₂₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, Rₓ is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b}— is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa):

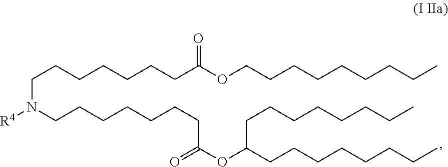

or their N-oxides, or salts or isomers thereof, wherein R⁴ is as described herein.

In another embodiment, the compounds of Formula (I) are of Formula (Ib):

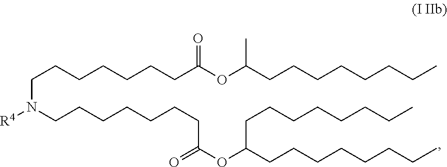

or their N-oxides, or salts or isomers thereof, wherein R⁴ is as described herein.

In another embodiment, the compounds of Formula (I) are of Formula (IIc) or (IIe):

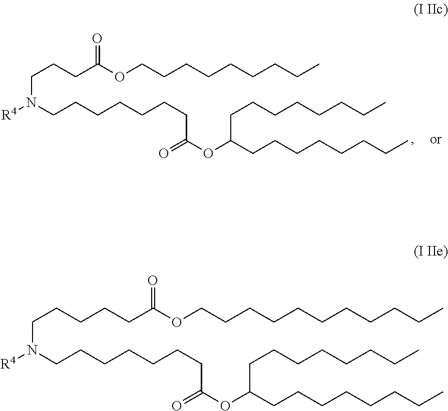

or their N-oxides, or salts or isomers thereof, wherein R⁴ is as described herein.

In another embodiment, the compounds of Formula (I I) are of Formula (I IIf):

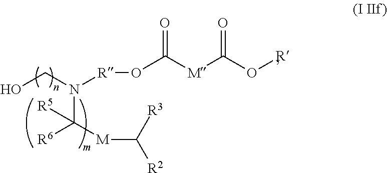

or their N-oxides, or salts or isomers thereof, wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

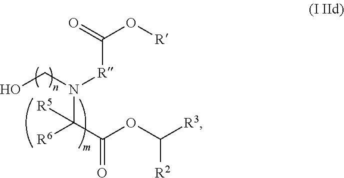

or their N-oxides, or salts or isomers thereof, wherein n is 2, 3, or 4; and m, R′, R″, and R² through R⁶ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

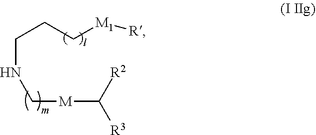

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(OXOR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

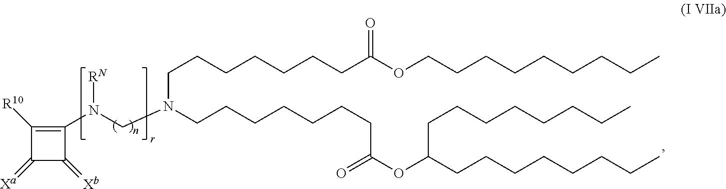

or its N-oxide, or a salt or isomer thereof.

In another embodiment, a subset of compounds of Formula (I VI) includes those of Formula (I VIIIa):

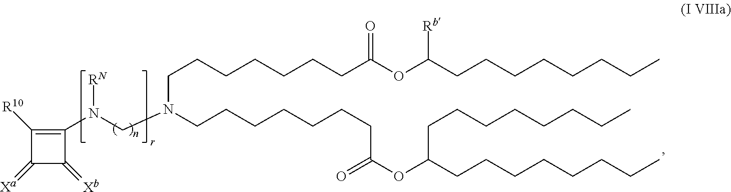

or its N-oxide, or a salt or isomer thereof.

In another embodiment, a subset of compounds of Formula (I VI) includes those of Formula I VIIIb):

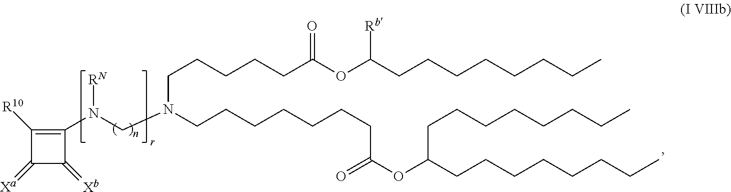

or its N-oxide, or a salt or isomer thereof.

In another embodiment, a subset of compounds of Formula (I VI) includes those of Formula (I VIIb-1):

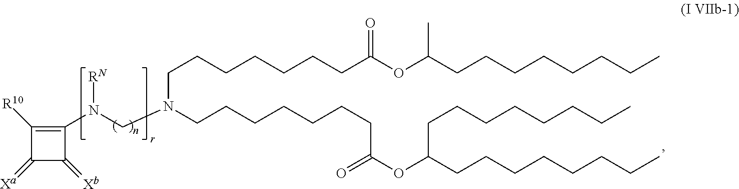

or its N-oxide, or a salt or isomer thereof.

In another embodiment, a subset of compounds of Formula (I VI) includes those of Formula (I VIIb-2):

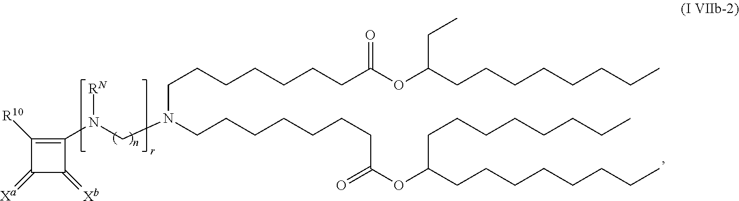

or its N-oxide, or a salt or isomer thereof.

In another embodiment, a subset of compounds of Formula (I VI) includes those of Formula (I VIIb-3):

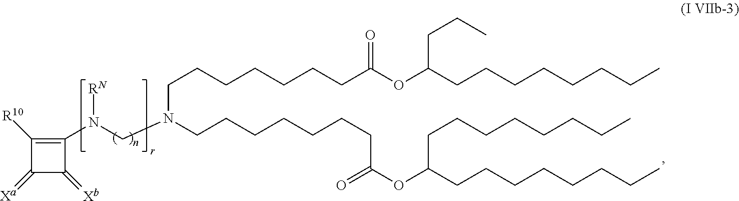

or its N-oxide, or a salt or isomer thereof. In another embodiment, a subset of compounds of Formula (VI) includes those of Formula (VIIc):

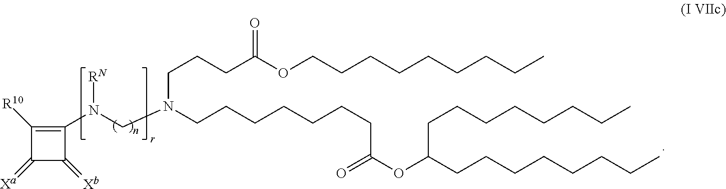

In another embodiment, a subset of compounds of Formula (I VI) includes those of Formula (VIId):

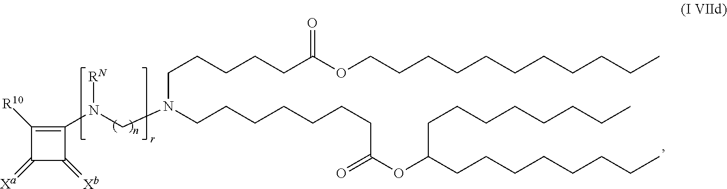

or its N-oxide, or a salt or isomer thereof.

In another embodiment, a subset of compounds of Formula (I VI) includes those of Formula (I VIIIc):

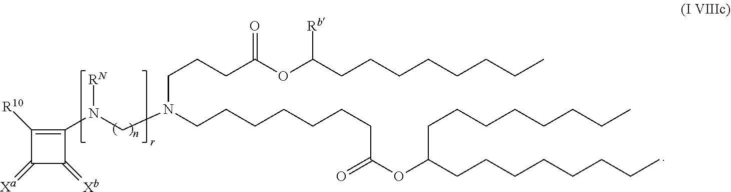

In another embodiment, a subset of compounds of Formula I VI) includes those of Formula (I VIIId):

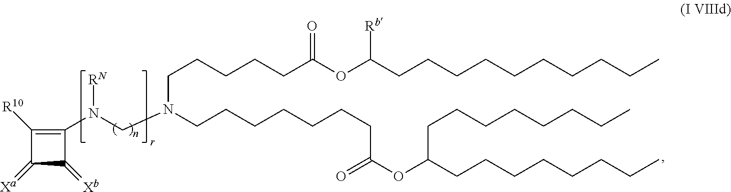

or its N-oxide, or a salt or isomer thereof.

The compounds of any one of formulae (I I), (I IA), (I IB), (I II), (I Ha), (I IIb), (I IIc), (I IId), (I IIe), (I IIf), (I IIg), I (III), (I VI), (I VI-a), (I VII), (I VIII), (I VIIa), (I VIIIa), (I VIII), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R″, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R8, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R″ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In another embodiment, R⁴ is is —(CH₂)ₒC(R¹⁰)₂(CH₂)ₙ₋ₒQ, where Q is —N(R)C(O)R, wherein R is C₁-C₃ alkyl, n is 3, and o is 1.

In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is C₁₋₃ alkyl or C₂₋₃ alkenyl. In another embodiment, each R¹⁰ is is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is OH.

In another embodiment, R⁴ is unsubstituted C₁₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₄ alkyl, C₂₋₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆ dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is Cis alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

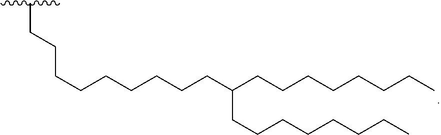 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

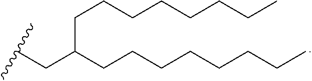 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

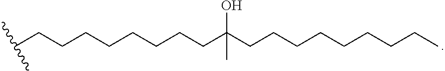 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

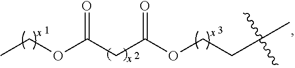

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ alkyl (e.g., C₃₋₁₂ alkyl substituted with, e.g., an hydroxyl). For example, R″ is

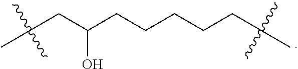 [SMILES: *CCCCCC(O)C*]

In some embodiments, R′ is selected from C4 alkyl and C4 alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl, each of which is either linear or branched.

In some embodiments, R′ is linear. In some embodiments, R′ is branched.

In some embodiments, R′ is

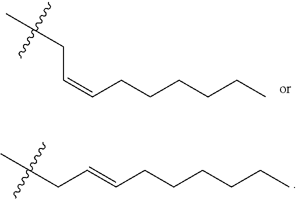

In some embodiments, R′ is

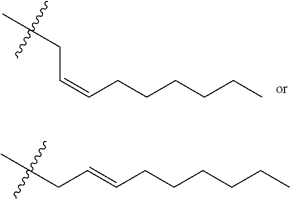

and M′ is —OC(O)—. In other embodiments, R′ is

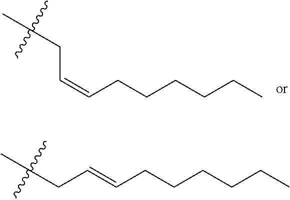

### or and M′ is —C(O)O—.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is In certain embodiments, R′ is unsubstituted C₁₋₁₅ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl or C(O)O-alkyl or OC(O)-alkyl such as C(O)OCH₃ or OC(O)CH₃). For example, R′ is

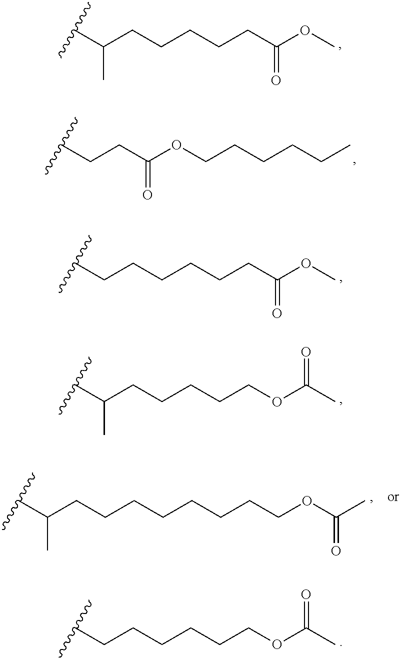 [SMILES: *C(C)CCCCCC(=O)OC ; *CCC(=O)OCCCCCC ; *CCCCCCC(=O)OC ; *C(C)CCCCCOC(C)=O ; *C(C)CCCCCCCCOC(C)=O ; *CCCCCCOC(C)=O]

In certain embodiments, R′ is branched C₁₋₁₈ alkyl. For example, R′ is

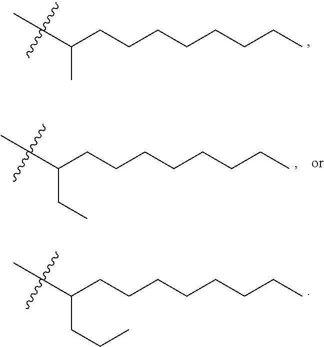 [SMILES: *C(C)CCCCCCCC ; *C(CC)CCCCCCCC ; *C(CCC)CCCCCCCC]

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, C₁₄ alkyl, or C₁₅ alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(OXOR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ alkyl (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR. In certain embodiments, Q is —N(OR)C(O)R, —N(OR)S(O)₂R,

—N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

In certain embodiments, Q is thiourea or an isostere thereof, e.g.,

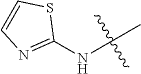 [SMILES: *Nc1nccs1]

or —NHC(═NR⁹)N(R)₂.

In certain embodiments, Q is —C(═NR⁹)N(R)₂. For example, when Q is —C(═NR⁹)N(R)₂, n is 4 or 5. For example, R⁹ is —S(O)₂N(R)₂.

In certain embodiments, Q is —C(═NR⁹)R or —C(O)N(R)OR, e.g.,
—CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R″ is a C₃₋₆ cycloalkyl optionally substituted with one or more substituents selected from oxo (═O), amino (NH2), mono- or di-alkylamino, C₁₋₃ alkyl and halo. For example, R″ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH2), mono- or di-alkylamino, C₁₋₃ alkyl, heterocycloalkyl, and halo, wherein the mono- or di-alkylamino, C₁₋₃ alkyl, and heterocycloalkyl are further substituted. For example, R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R″ is 3-(((dimethylamino)ethyl)amino)cyclobut-3-enyl-1,2-dione. For example, R″ is cyclobutenyl substituted with one or more of oxo, and alkylamino. For example, R″ is 3-(ethylamino)cyclobut-3-ene-1,2-dione. For example, R″ is cyclobutenyl substituted with one or more of oxo, thio, and alkylamino. For example, R⁸ is 3-(ethylamino)-4-thioxocyclobut-2-en-1-one or 2-(ethylamino)-4-thioxocyclobut-2-en-1-one. For example, R″ is cyclobutenyl substituted with one or more of thio, and alkylamino. For example, R″ is 3-(ethylamino)cyclobut-3-ene-1,2-dithione. For example, R″ is cyclobutenyl substituted with one or more of oxo and dialkylamino. For example, R⁸ is 3-(diethylamino)cyclobut-3-ene-1,2-dione. For example, R″ is cyclobutenyl substituted with one or more of oxo, thio, and dialkylamino. For example, R″ is 2-(diethylamino)-4-thioxocyclobut-2-en-1-one or 3-(diethylamino)-4-thioxocyclobut-2-en-1-one. For example, R″ is cyclobutenyl substituted with one or more of thio, and dialkylamino. For example, R⁸ is 3-(diethylamino)cyclobut-3-ene-1,2-dithione. For example, R″ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R″ is cyclobutenyl substituted with one or more of oxo, and heterocycloalkyl. For example, R″ is cyclobutenyl substituted with one or more of oxo, and piperidinyl, piperazinyl, or morpholinyl. For example, R″ is cyclobutenyl substituted with one or more of oxo, and heterocycloalkyl, wherein heterocycloalkyl is further substituted, e.g., with one or more C₁₋₃ alkyl. For example, R″ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R″ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂,

—NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O(CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

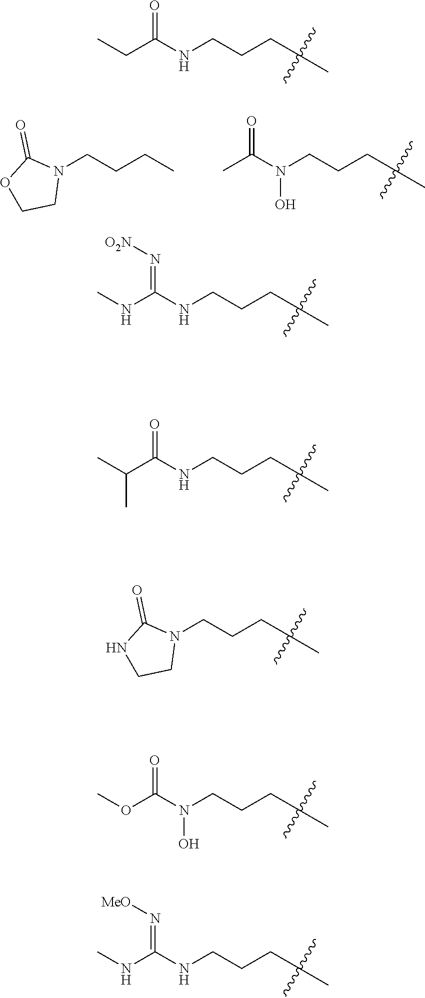

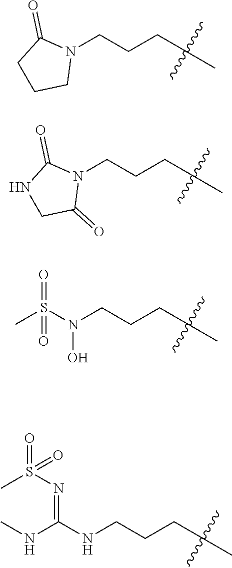

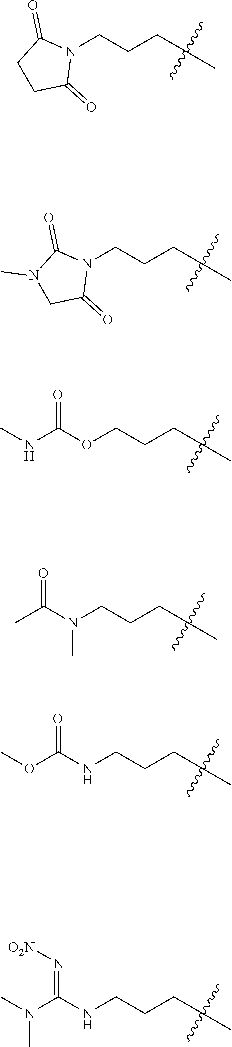

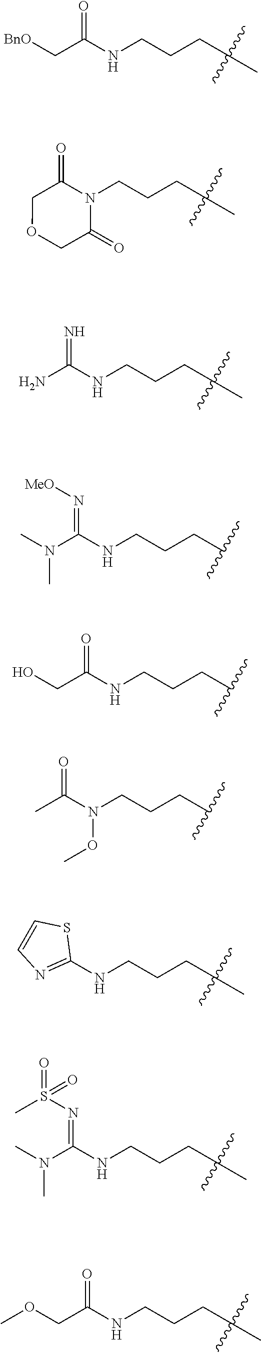

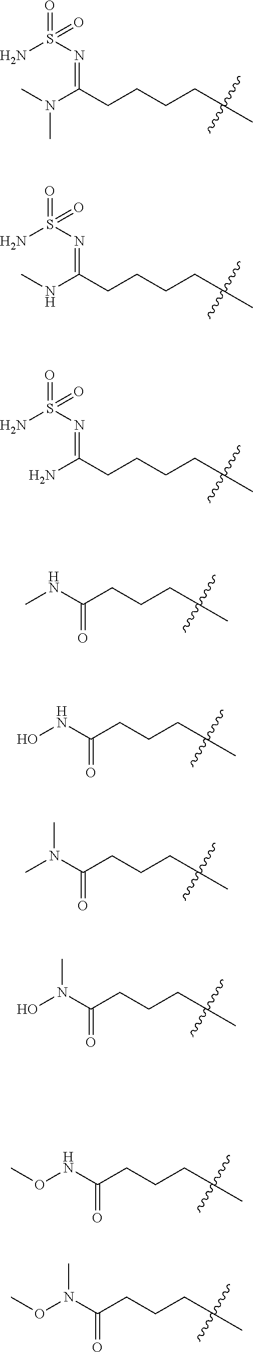

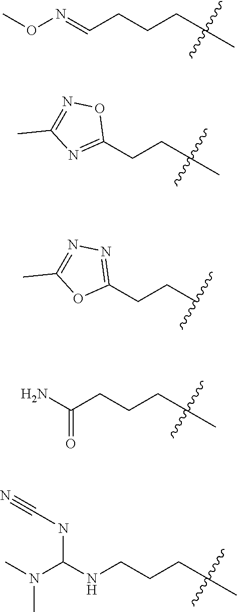

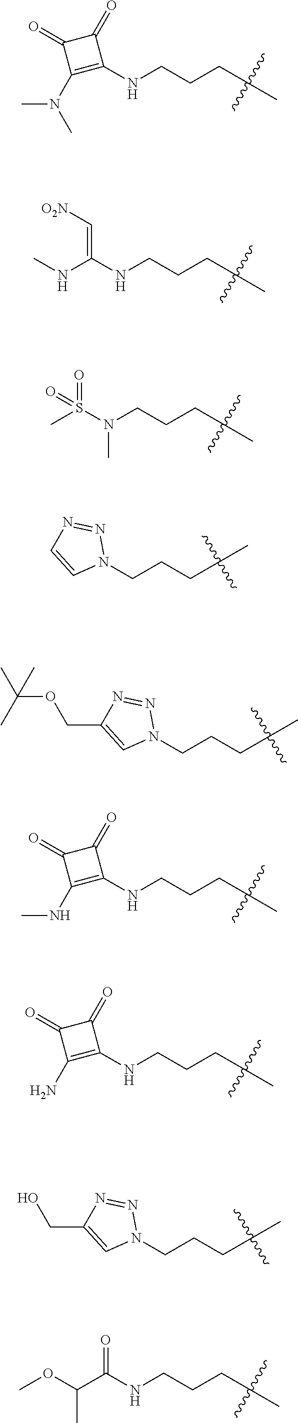

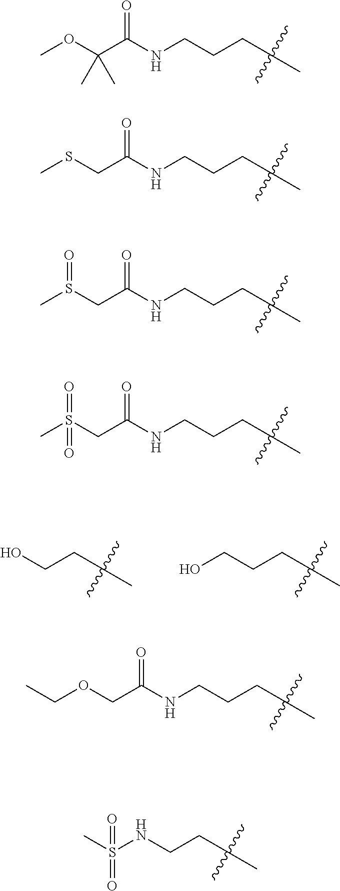

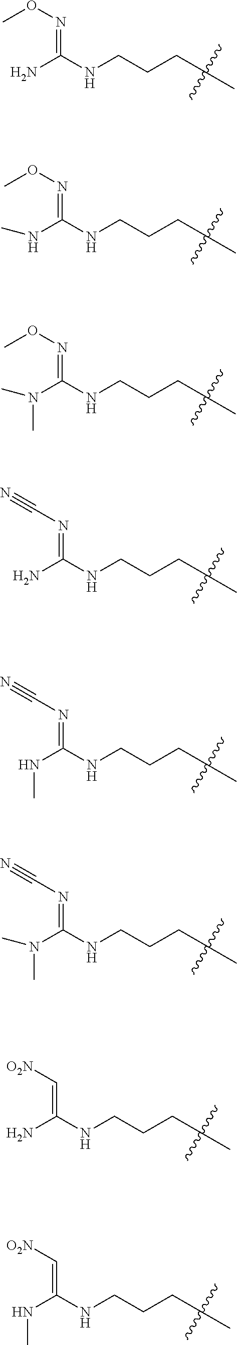

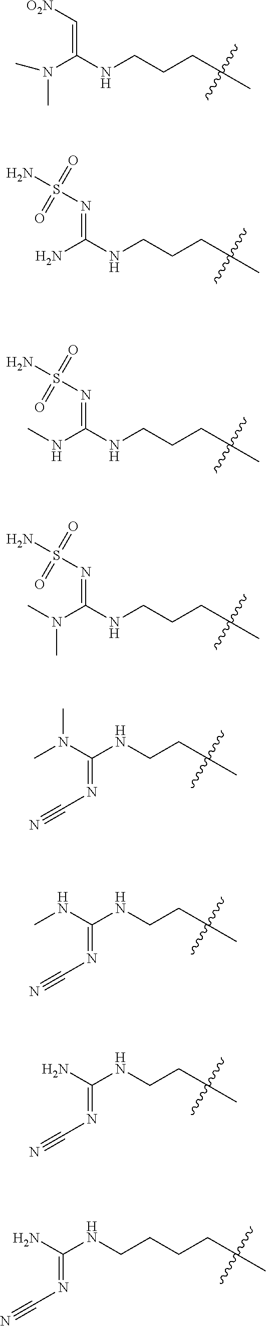

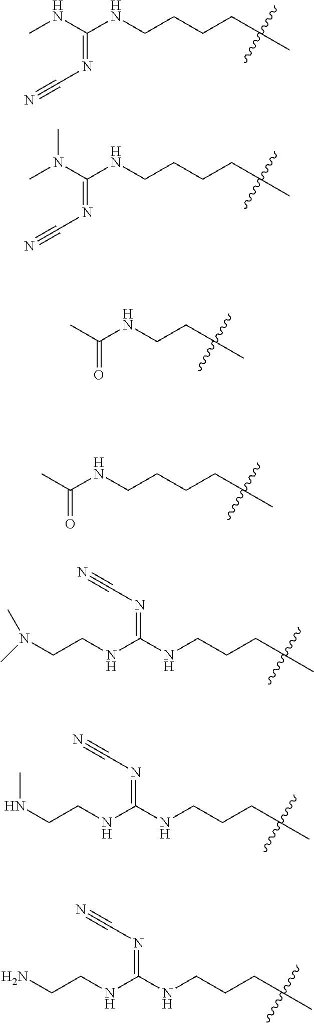

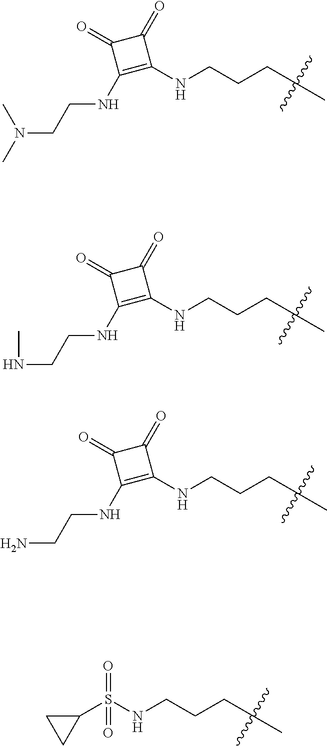 [SMILES: *CCCNc1c(NCCN(C)C)c(=O)c1=O ; *CCCNc1c(NCCNC)c(=O)c1=O ; *CCCNc1c(NCCN)c(=O)c1=O ; *CCCNS(=O)(=O)C1CC1]

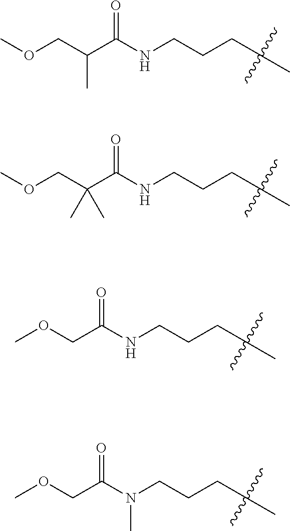

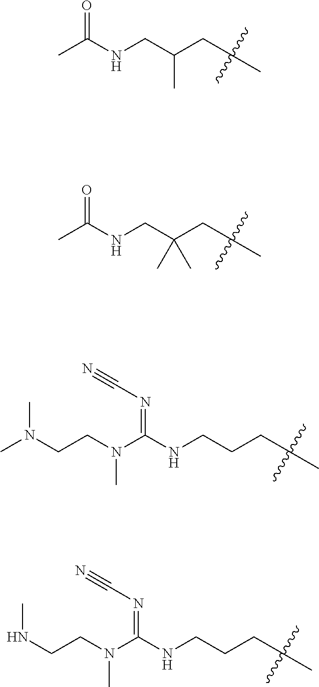

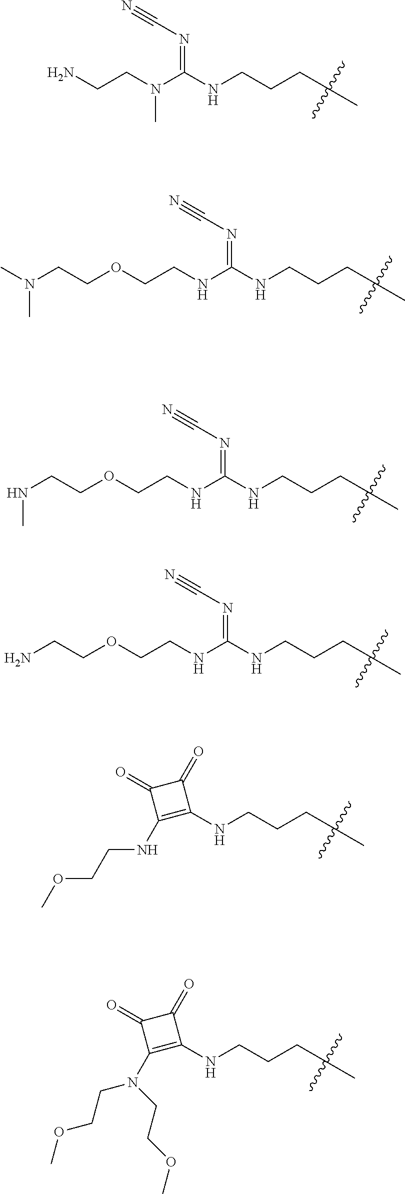 [SMILES: *CCCN/C(=N/C#N)N(C)CCN ; *CCCN/C(=N/C#N)NCCOCCN(C)C ; *CCCN/C(=N/C#N)NCCOCCNC ; *CCCN/C(=N/C#N)NCCOCCN ; *CCCNc1c(NCCOC)c(=O)c1=O ; *CCCNc1c(N(CCOC)CCOC)c(=O)c1=O]

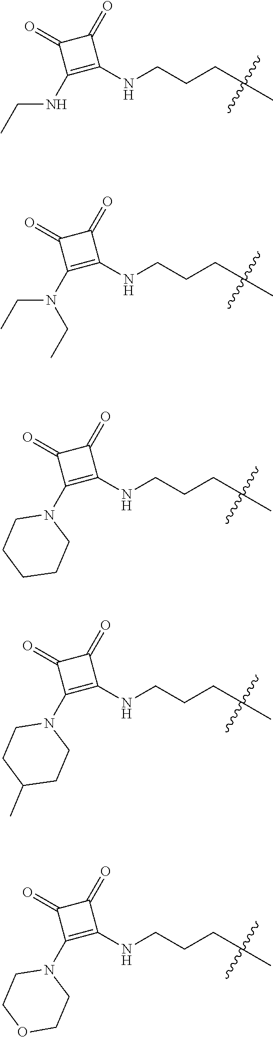 [SMILES: *CCCNc1c(NCC)c(=O)c1=O ; *CCCNc1c(N(CC)CC)c(=O)c1=O ; *CCCNc1c(N2CCCCC2)c(=O)c1=O ; *CCCNc1c(N2CCC(C)CC2)c(=O)c1=O ; *CCCNc1c(N2CCOCC2)c(=O)c1=O]

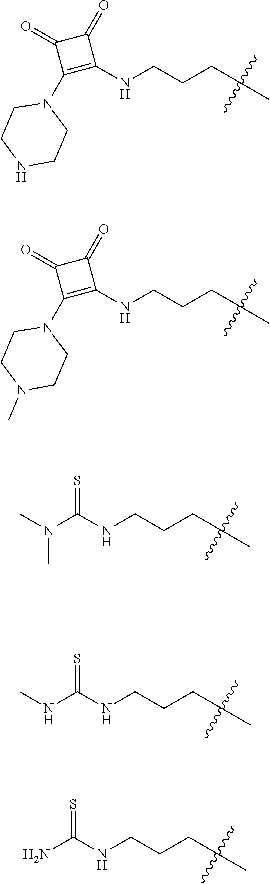

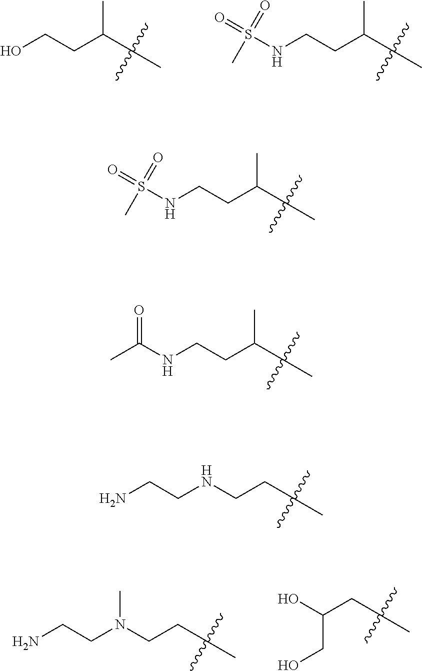

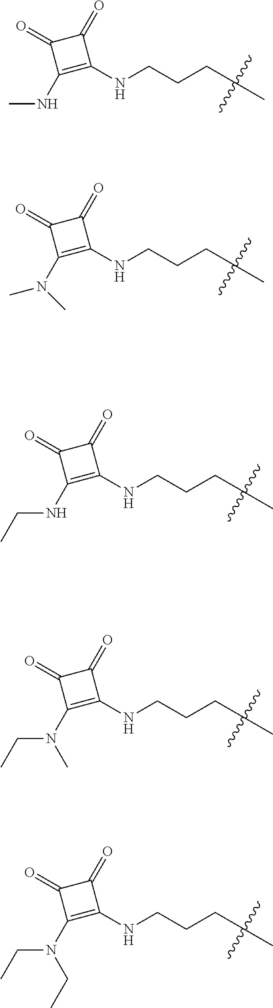 [SMILES: *CCCNc1c(NC)c(=O)c1=O ; *CCCNc1c(N(C)C)c(=O)c1=O ; *CCCNc1c(NCC)c(=O)c1=O ; *CCCNc1c(N(C)CC)c(=O)c1=O ; *CCCNc1c(N(CC)CC)c(=O)c1=O]

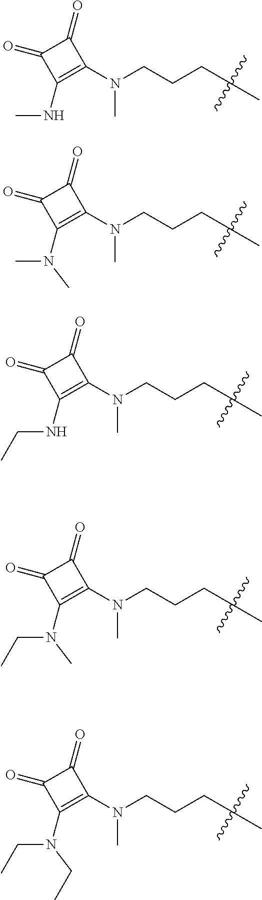

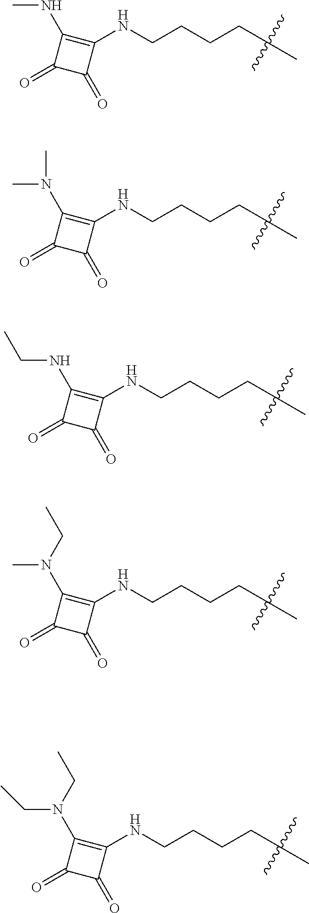 [SMILES: *CCCCNc1c(NC)c(=O)c1=O ; *CCCCNc1c(N(C)C)c(=O)c1=O ; *CCCCNc1c(NCC)c(=O)c1=O ; *CCCCNc1c(N(C)CC)c(=O)c1=O ; *CCCCNc1c(N(CC)CC)c(=O)c1=O]

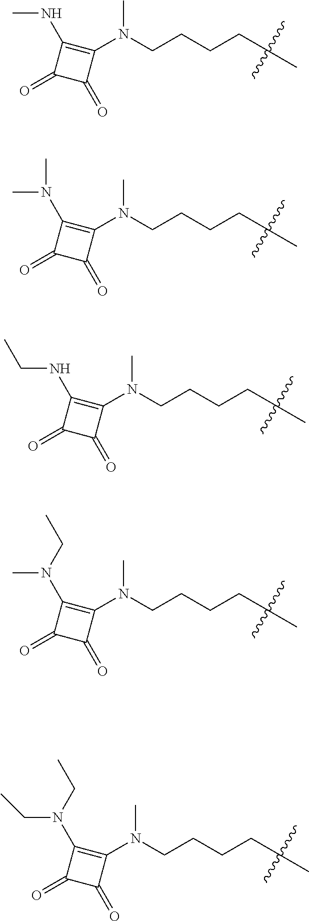 [SMILES: *CCCCN(C)c1c(NC)c(=O)c1=O ; *CCCCN(C)c1c(N(C)C)c(=O)c1=O ; *CCCCN(C)c1c(NCC)c(=O)c1=O ; *CCCCN(C)c1c(N(C)CC)c(=O)c1=O ; *CCCCN(C)c1c(N(CC)CC)c(=O)c1=O]

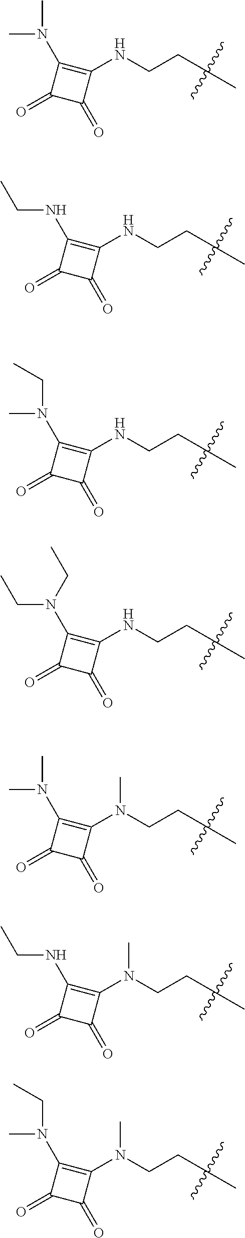

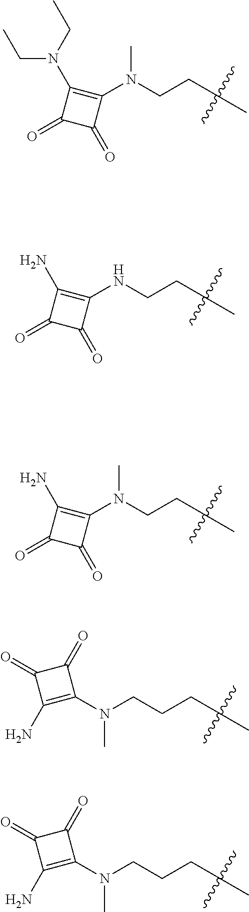 [SMILES: *CCN(C)c1c(N(CC)CC)c(=O)c1=O ; *CCNc1c(N)c(=O)c1=O ; *CCN(C)c1c(N)c(=O)c1=O ; *CCCN(C)c1c(N)c(=O)c1=O ; *CCCN(C)c1c(N)c(=O)c1=O]

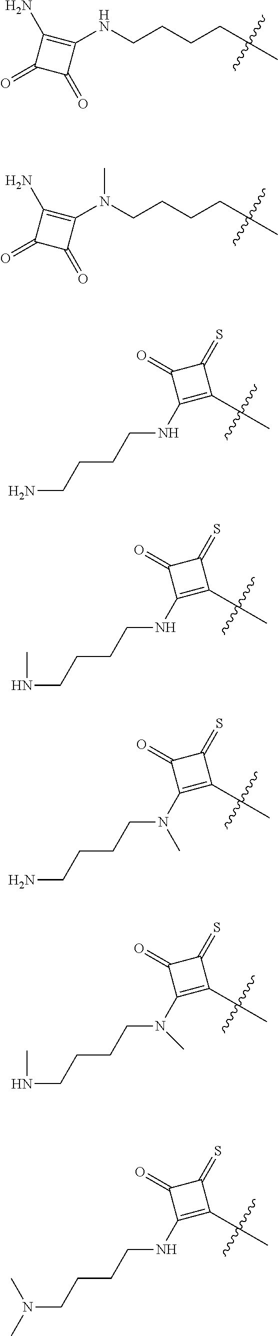

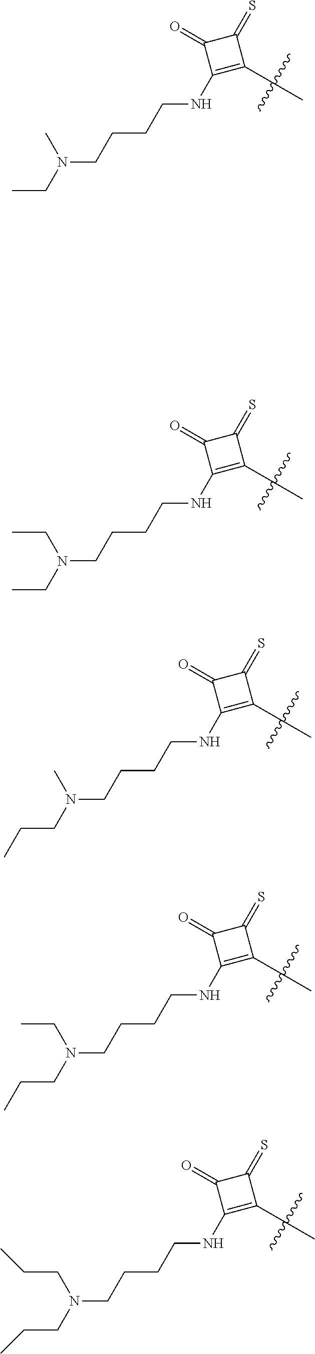 [SMILES: *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

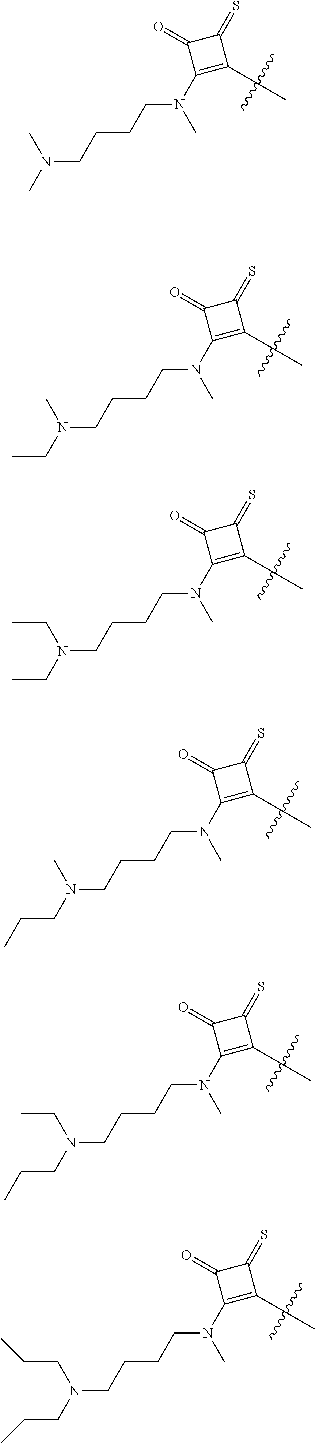 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

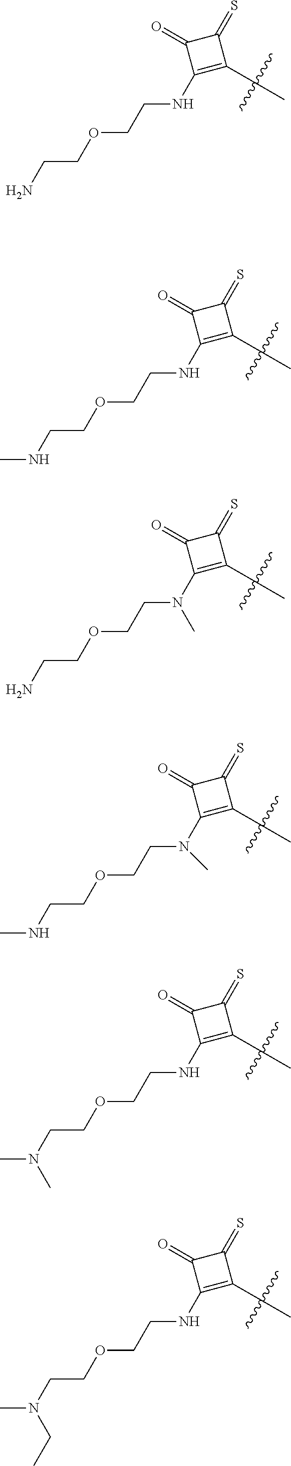 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S ; *c1c(NCCOCCN(C)C)c(=O)c1=S ; *c1c(NCCOCCN(C)CC)c(=O)c1=S]

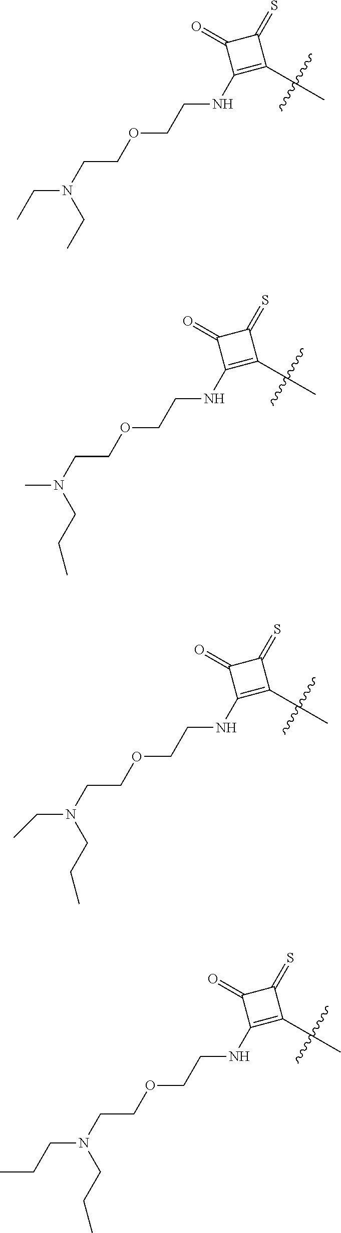 [SMILES: *c1c(NCCOCCN(CC)CC)c(=O)c1=S ; *c1c(NCCOCCN(C)CCC)c(=O)c1=S ; *c1c(NCCOCCN(CC)CCC)c(=O)c1=S ; *c1c(NCCOCCN(CCC)CCC)c(=O)c1=S]

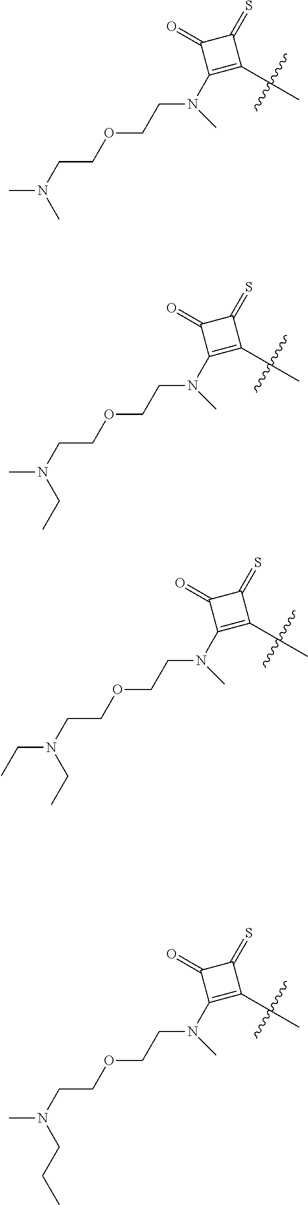 [SMILES: *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

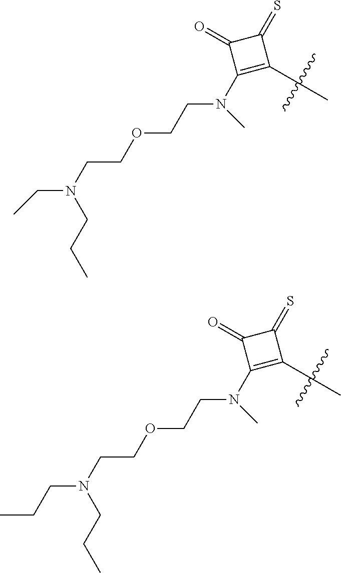 [SMILES: *c1c(N(C)CCOCCN(CC)CCC)c(=O)c1=S ; *c1c(N(C)CCOCCN(CCC)CCC)c(=O)c1=S]

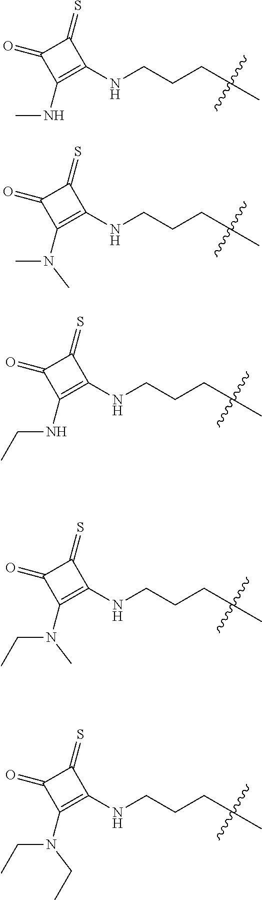

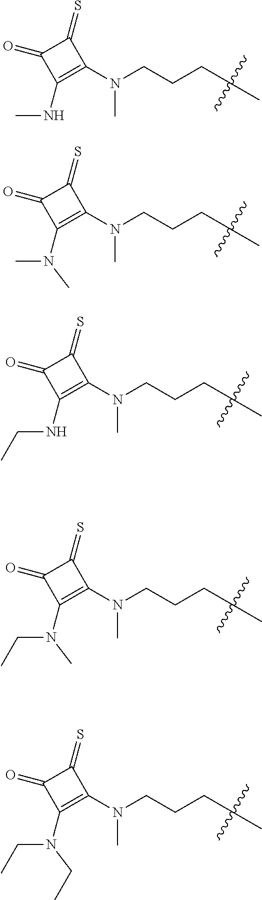

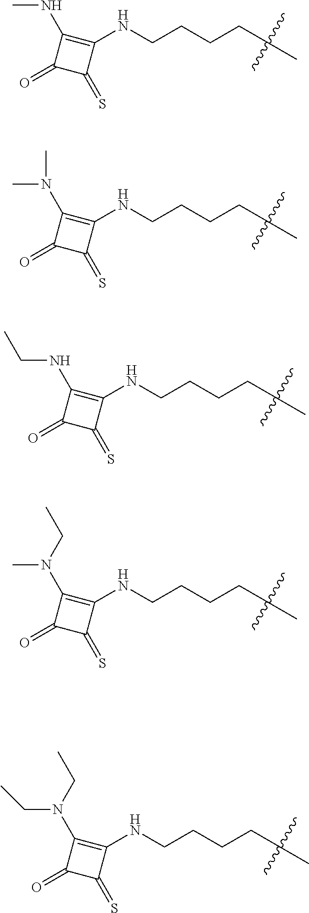 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

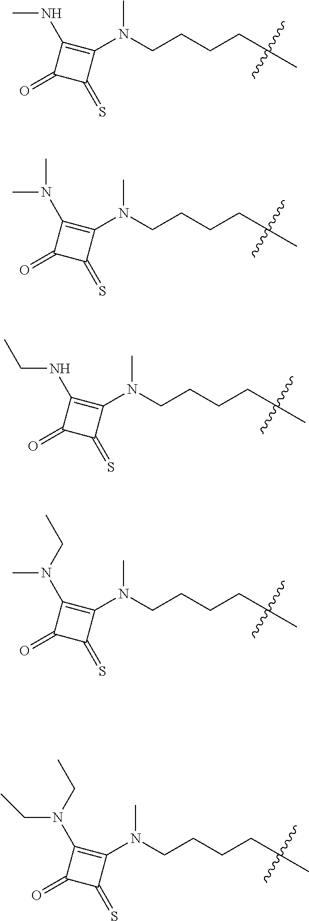 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

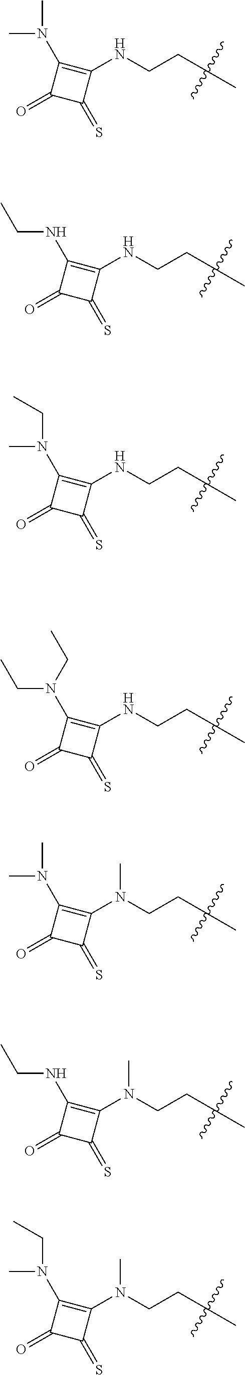

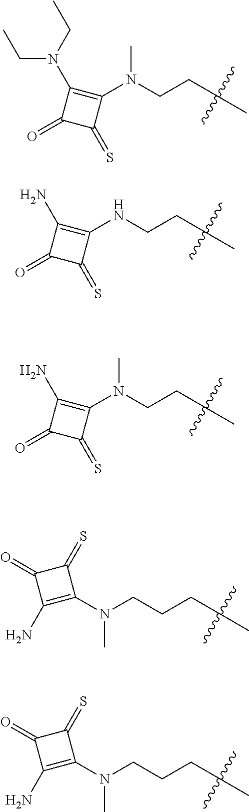 [SMILES: *CCN(C)c1c(N(CC)CC)c(=O)c1=S ; *CCNc1c(N)c(=O)c1=S ; *CCN(C)c1c(N)c(=O)c1=S ; *CCCN(C)c1c(N)c(=O)c1=S ; *CCCN(C)c1c(N)c(=O)c1=S]

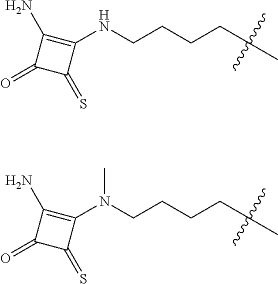 [SMILES: *CCCCNc1c(N)c(=O)c1=S ; *CCCCN(C)c1c(N)c(=O)c1=S]

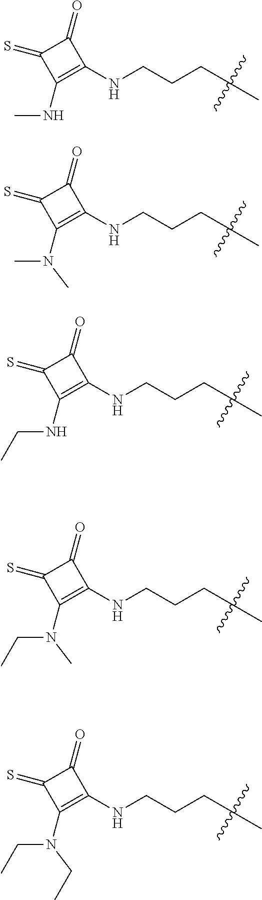

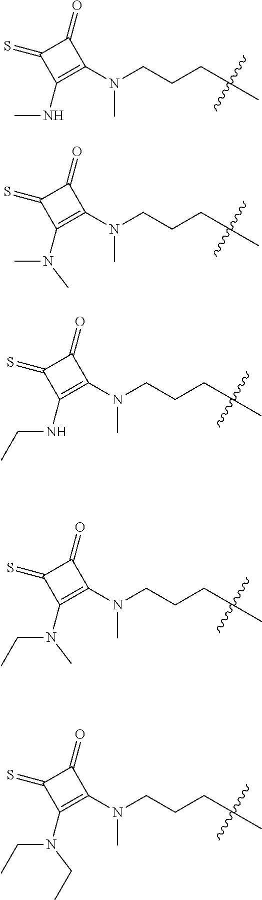

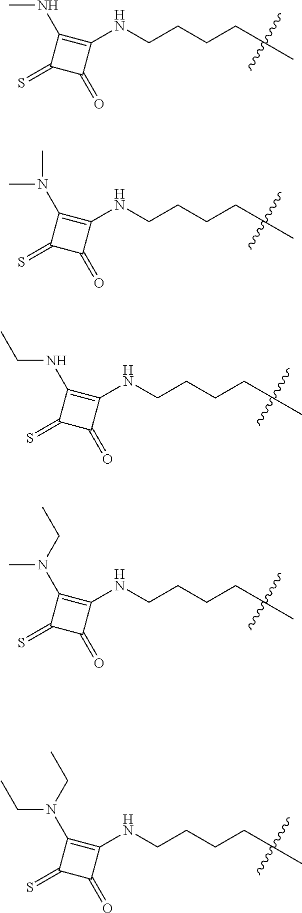 [SMILES: *CCCCNc1c(NC)c(=S)c1=O ; *CCCCNc1c(N(C)C)c(=S)c1=O ; *CCCCNc1c(NCC)c(=S)c1=O ; *CCCCNc1c(N(C)CC)c(=S)c1=O ; *CCCCNc1c(N(CC)CC)c(=S)c1=O]

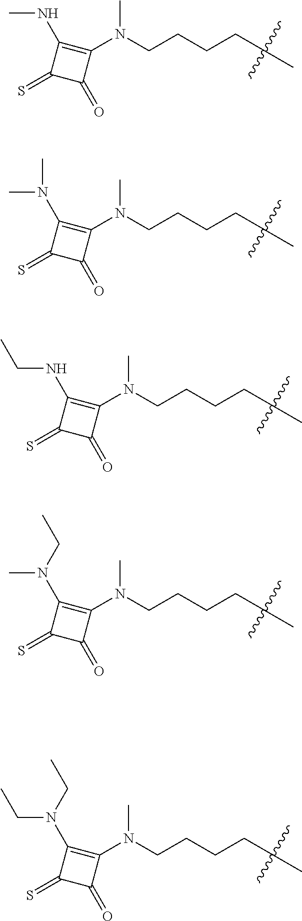 [SMILES: *CCCCN(C)c1c(NC)c(=S)c1=O ; *CCCCN(C)c1c(N(C)C)c(=S)c1=O ; *CCCCN(C)c1c(NCC)c(=S)c1=O ; *CCCCN(C)c1c(N(C)CC)c(=S)c1=O ; *CCCCN(C)c1c(N(CC)CC)c(=S)c1=O]

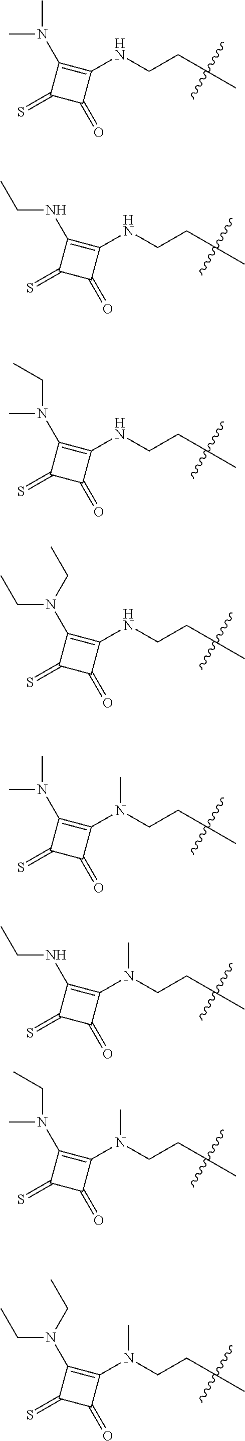

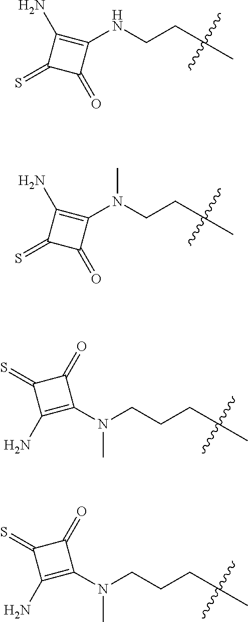 [SMILES: *CCNc1c(N)c(=S)c1=O ; *CCN(C)c1c(N)c(=S)c1=O ; *CCCN(C)c1c(N)c(=S)c1=O ; *CCCN(C)c1c(N)c(=S)c1=O]

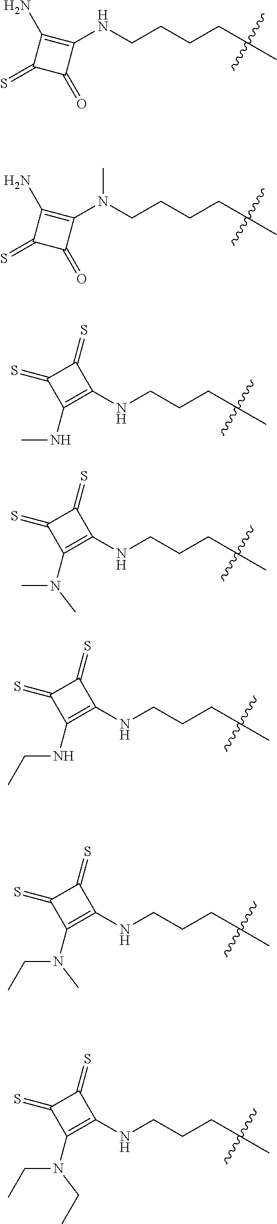

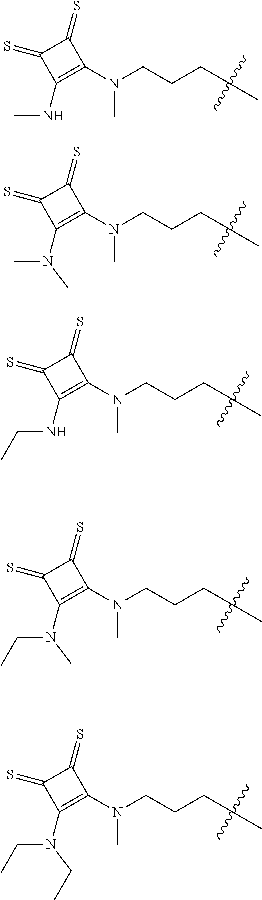

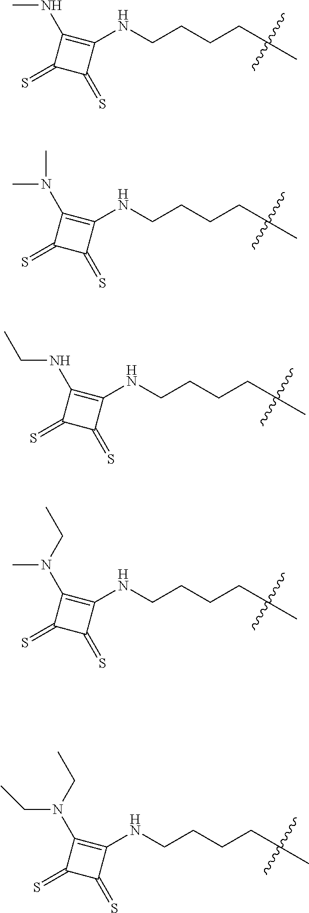 [SMILES: *CCCCNc1c(NC)c(=S)c1=S ; *CCCCNc1c(N(C)C)c(=S)c1=S ; *CCCCNc1c(NCC)c(=S)c1=S ; *CCCCNc1c(N(C)CC)c(=S)c1=S ; *CCCCNc1c(N(CC)CC)c(=S)c1=S]

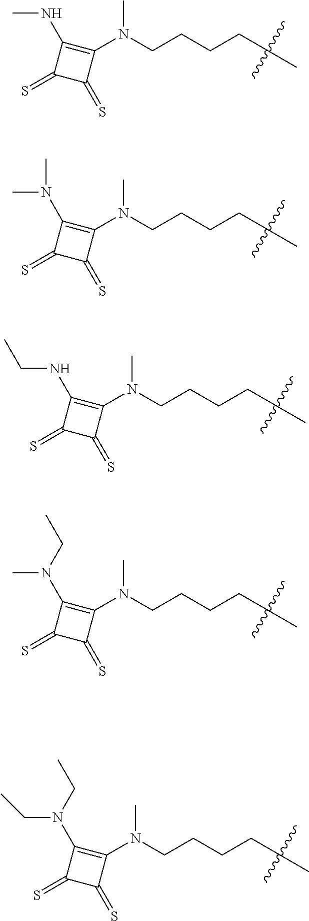 [SMILES: *CCCCN(C)c1c(NC)c(=S)c1=S ; *CCCCN(C)c1c(N(C)C)c(=S)c1=S ; *CCCCN(C)c1c(NCC)c(=S)c1=S ; *CCCCN(C)c1c(N(C)CC)c(=S)c1=S ; *CCCCN(C)c1c(N(CC)CC)c(=S)c1=S]

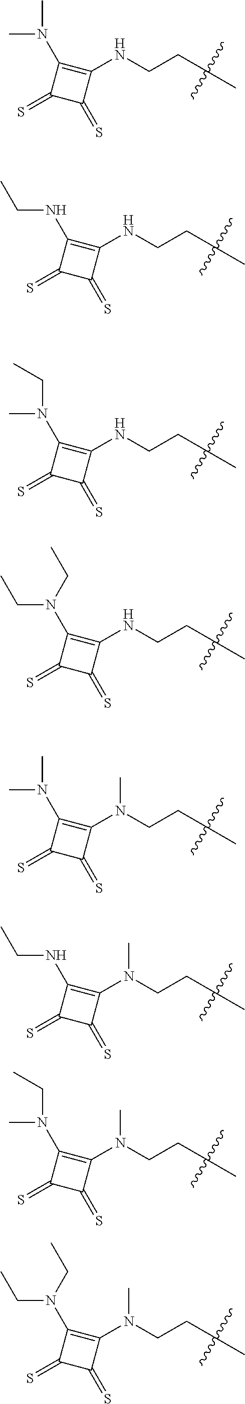

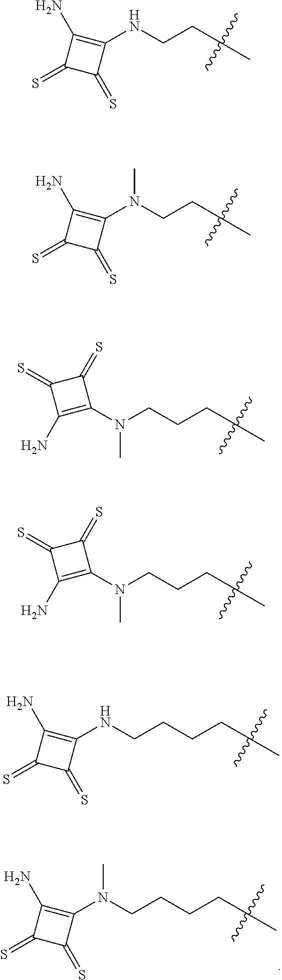 [SMILES: *CCNc1c(N)c(=S)c1=S ; *CCN(C)c1c(N)c(=S)c1=S ; *CCCN(C)c1c(N)c(=S)c1=S ; *CCCN(C)c1c(N)c(=S)c1=S ; *CCCCNc1c(N)c(=S)c1=S ; *CCCCN(C)c1c(N)c(=S)c1=S]

In some embodiments,

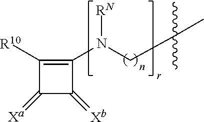

is selected from any of the following groups:

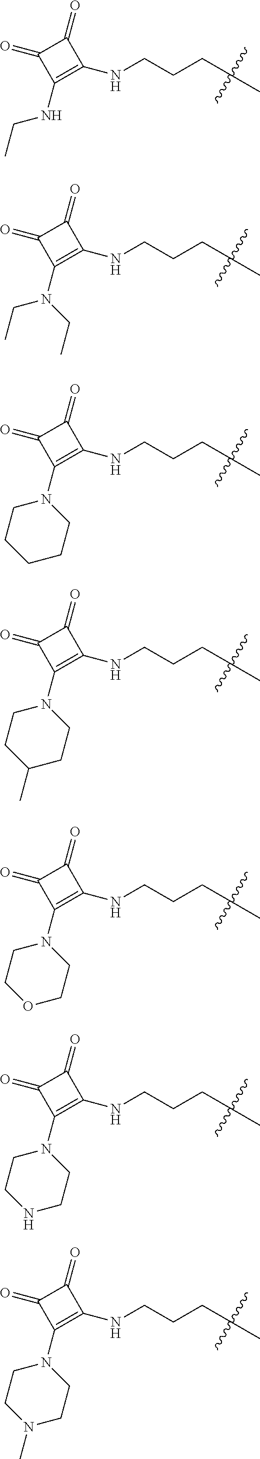

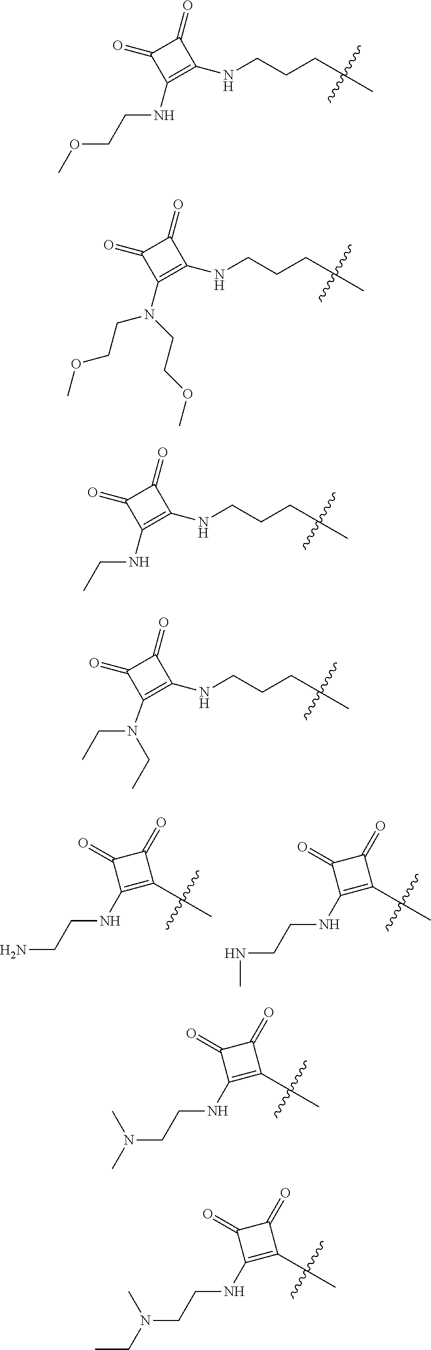

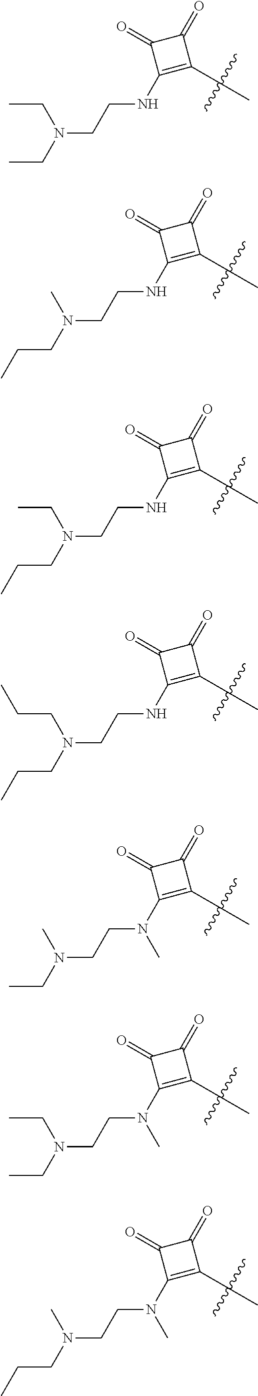

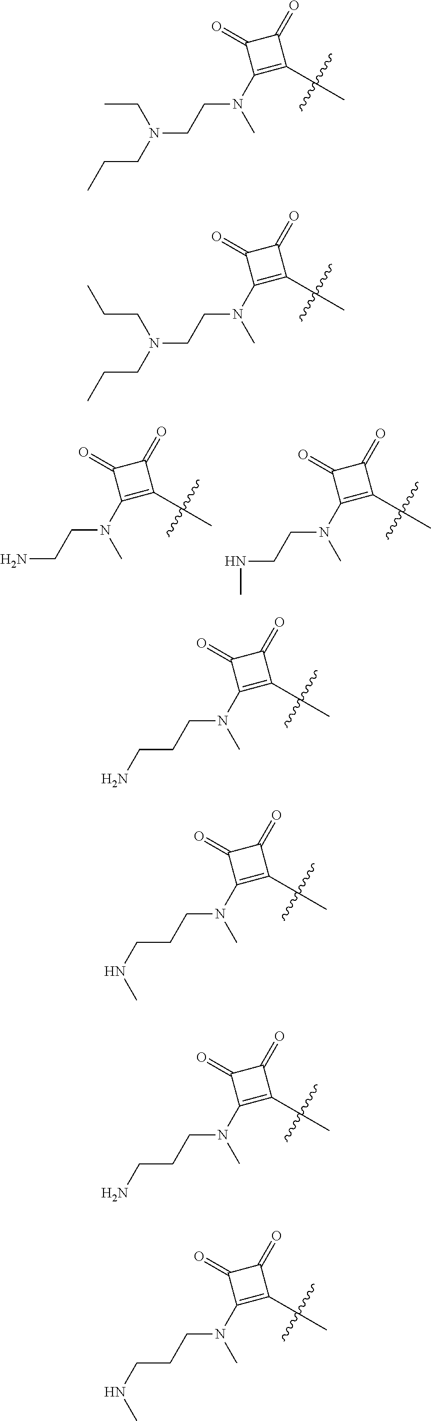

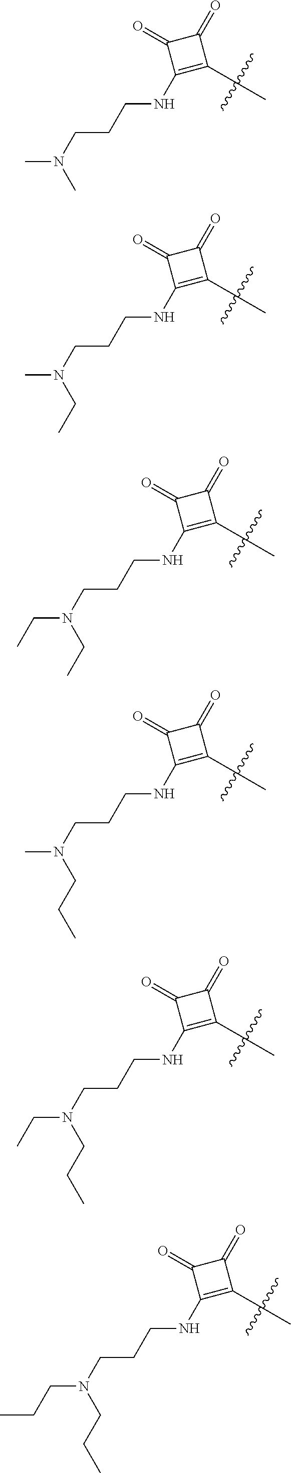 [SMILES: *c1c(NCCCN(C)C)c(=O)c1=O ; *c1c(NCCCN(C)CC)c(=O)c1=O ; *c1c(NCCCN(CC)CC)c(=O)c1=O ; *c1c(NCCCN(C)CCC)c(=O)c1=O ; *c1c(NCCCN(CC)CCC)c(=O)c1=O ; *c1c(NCCCN(CCC)CCC)c(=O)c1=O]

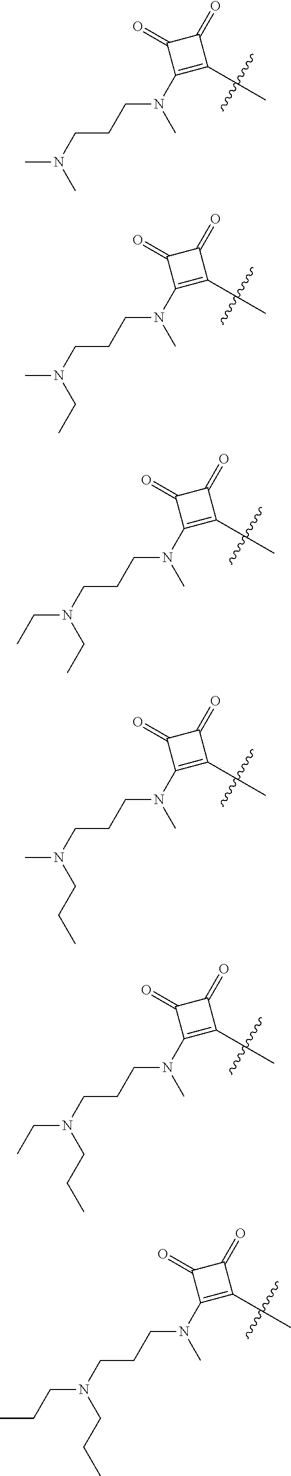 [SMILES: *c1c(N(C)CCCN(C)C)c(=O)c1=O ; *c1c(N(C)CCCN(C)CC)c(=O)c1=O ; *c1c(N(C)CCCN(CC)CC)c(=O)c1=O ; *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O]

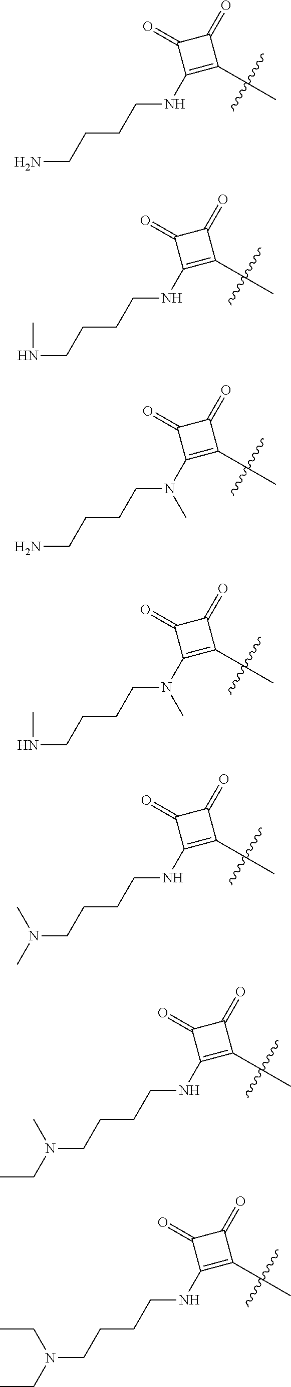

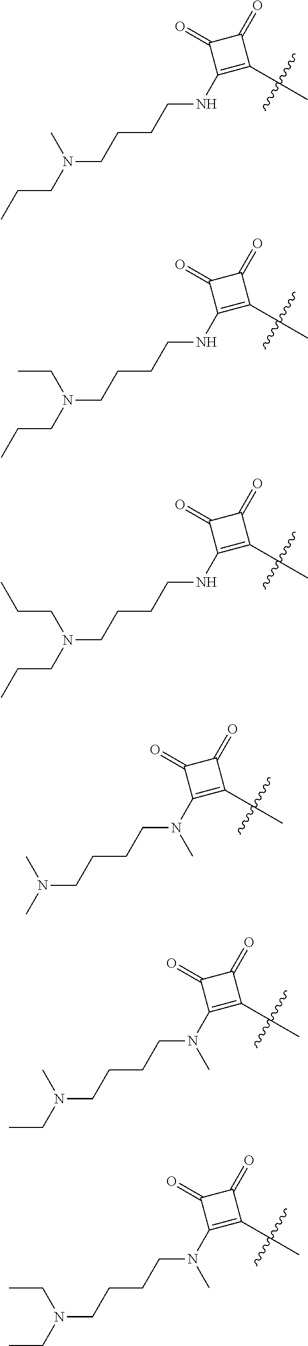 [SMILES: *c1c(NCCCCN(C)CCC)c(=O)c1=O ; *c1c(NCCCCN(CC)CCC)c(=O)c1=O ; *c1c(NCCCCN(CCC)CCC)c(=O)c1=O ; *c1c(N(C)CCCCN(C)C)c(=O)c1=O ; *c1c(N(C)CCCCN(C)CC)c(=O)c1=O ; *c1c(N(C)CCCCN(CC)CC)c(=O)c1=O]

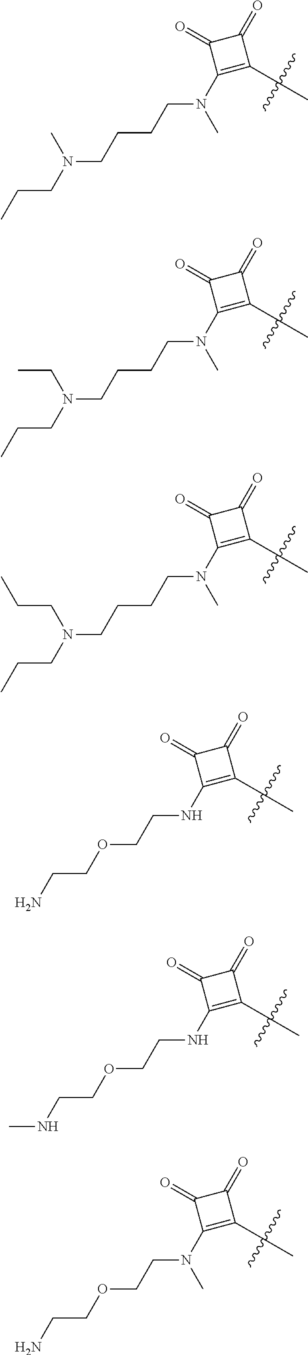 [SMILES: *c1c(N(C)CCCCN(C)CCC)c(=O)c1=O ; *c1c(N(C)CCCCN(CC)CCC)c(=O)c1=O ; *c1c(N(C)CCCCN(CCC)CCC)c(=O)c1=O ; *c1c(NCCOCCN)c(=O)c1=O ; *c1c(NCCOCCNC)c(=O)c1=O ; *c1c(N(C)CCOCCN)c(=O)c1=O]

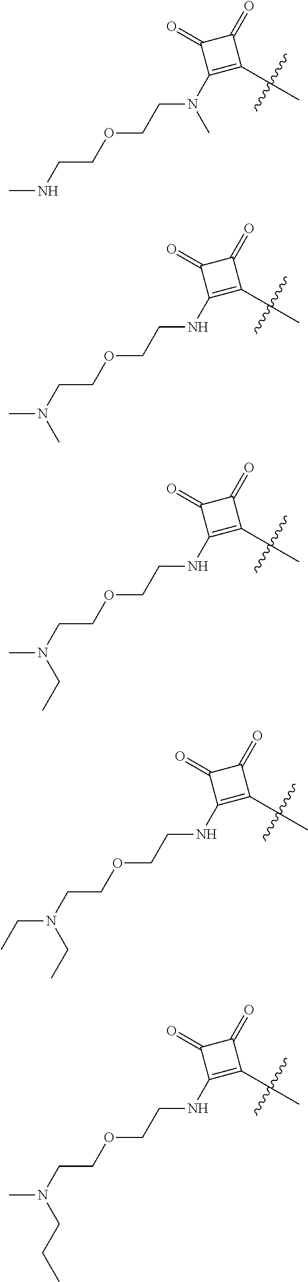 [SMILES: *c1c(N(C)CCOCCNC)c(=O)c1=O ; *c1c(NCCOCCN(C)C)c(=O)c1=O ; *c1c(NCCOCCN(C)CC)c(=O)c1=O ; *c1c(NCCOCCN(CC)CC)c(=O)c1=O ; *c1c(NCCOCCN(C)CCC)c(=O)c1=O]

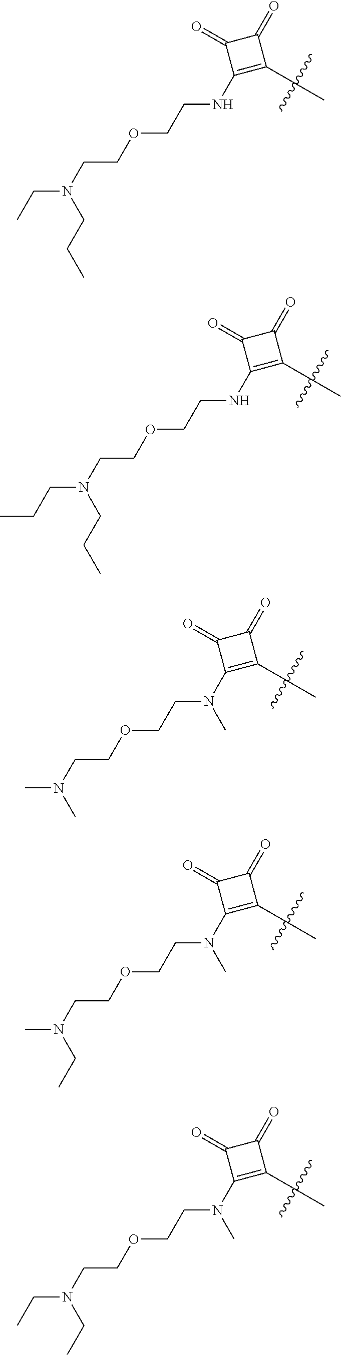 [SMILES: *c1c(NCCOCCN(CC)CCC)c(=O)c1=O ; *c1c(NCCOCCN(CCC)CCC)c(=O)c1=O ; *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O]

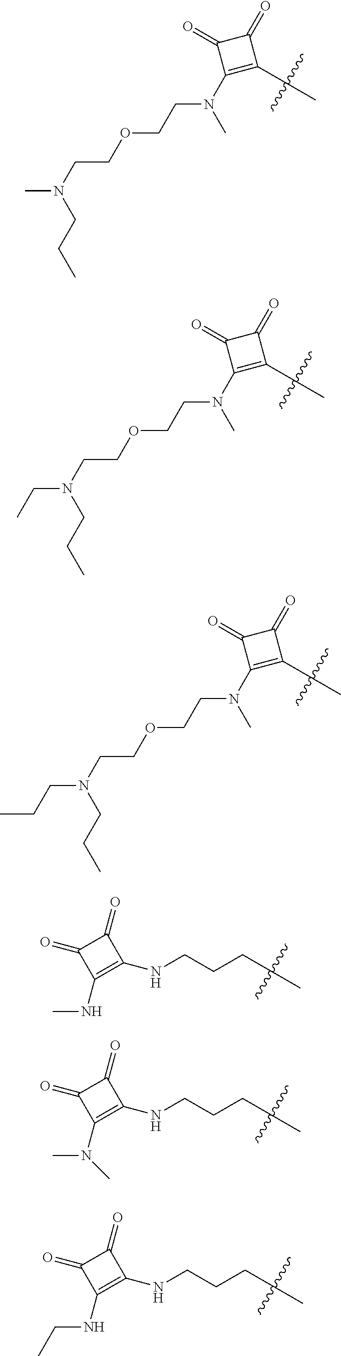

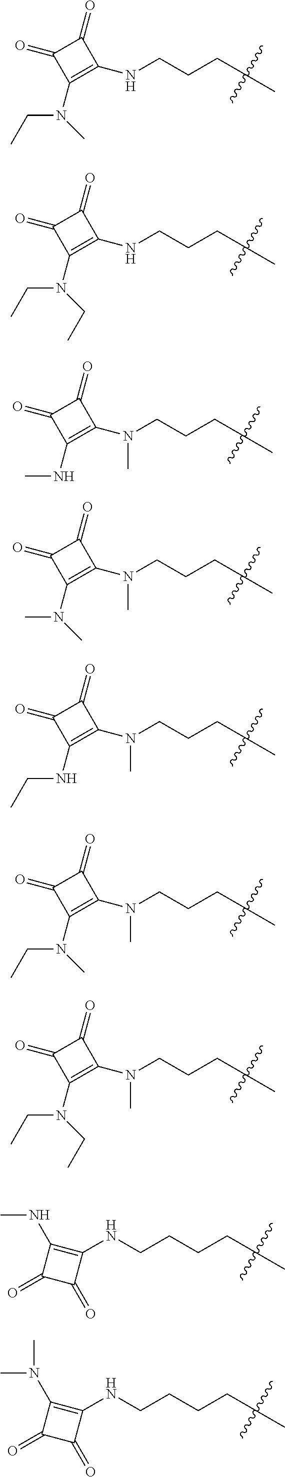

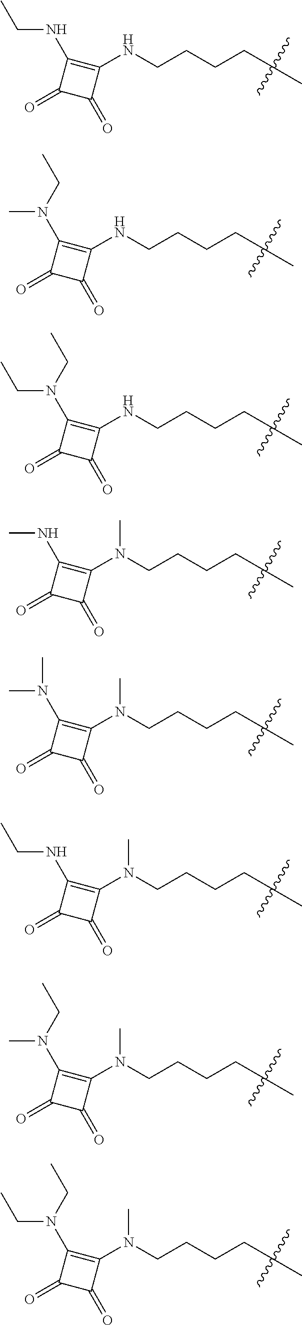

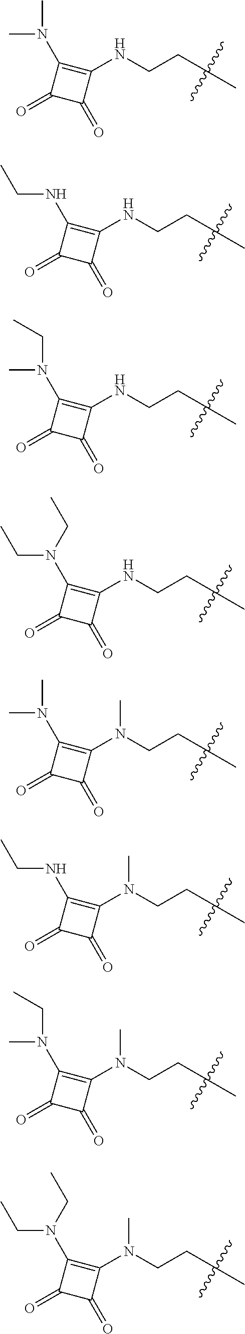

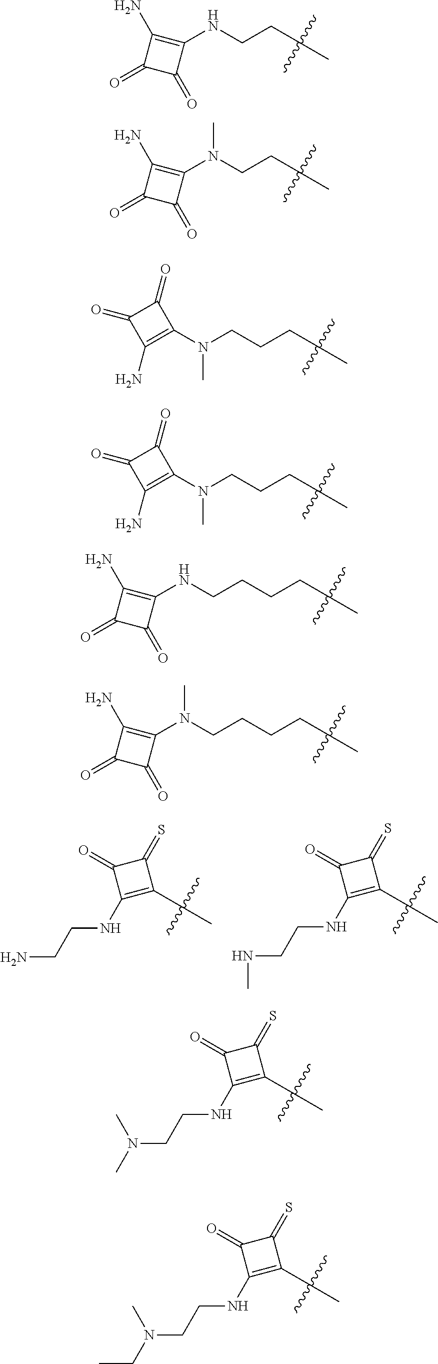

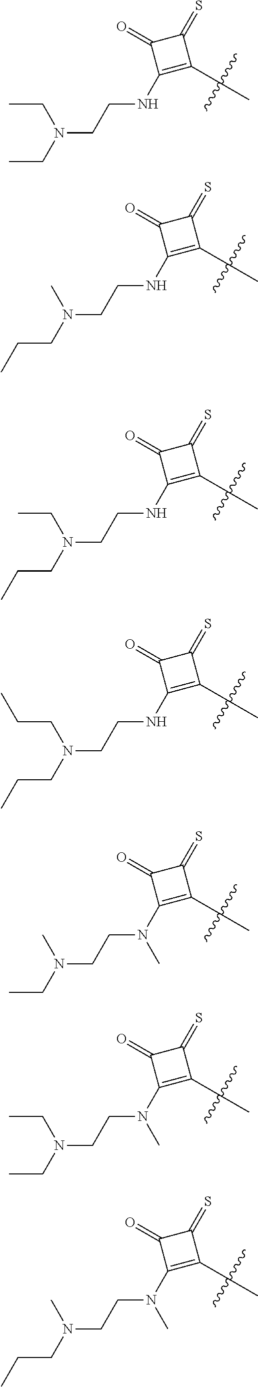

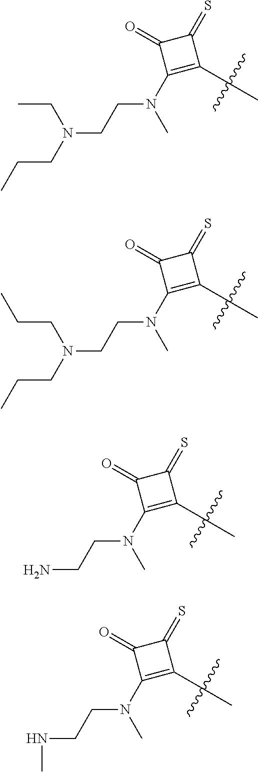 [SMILES: *c1c(N(C)CCN(CC)CCC)c(=O)c1=S ; *c1c(N(C)CCN(CCC)CCC)c(=O)c1=S ; *c1c(N(C)CCN)c(=O)c1=S ; *c1c(N(C)CCNC)c(=O)c1=S]

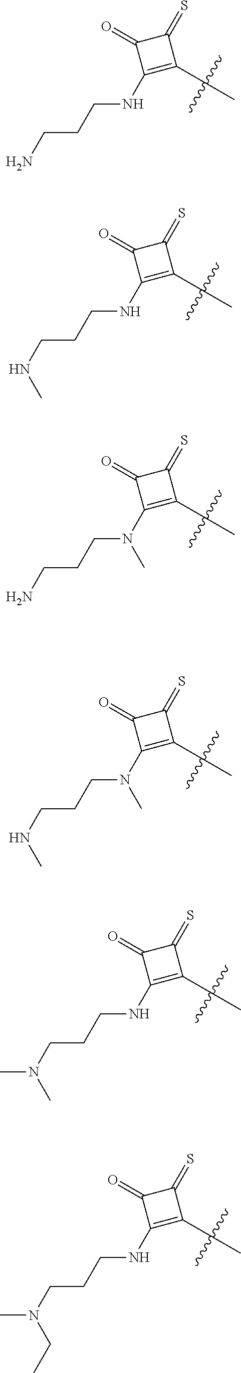 [SMILES: *c1c(NCCCN)c(=O)c1=S ; *c1c(NCCCNC)c(=O)c1=S ; *c1c(N(C)CCCN)c(=O)c1=S ; *c1c(N(C)CCCNC)c(=O)c1=S ; *c1c(NCCCN(C)C)c(=O)c1=S ; *c1c(NCCCN(C)CC)c(=O)c1=S]

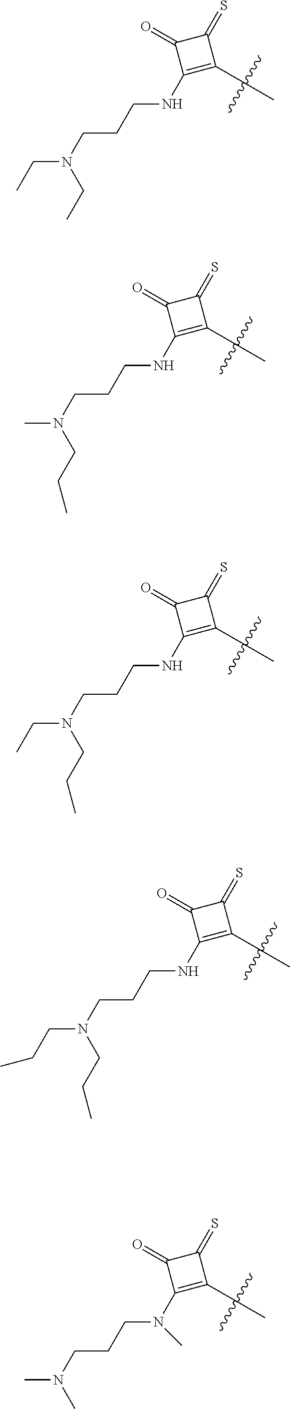 [SMILES: *c1c(NCCCN(CC)CC)c(=O)c1=S ; *c1c(NCCCN(C)CCC)c(=O)c1=S ; *c1c(NCCCN(CC)CCC)c(=O)c1=S ; *c1c(NCCCN(CCC)CCC)c(=O)c1=S ; *c1c(N(C)CCCN(C)C)c(=O)c1=S]

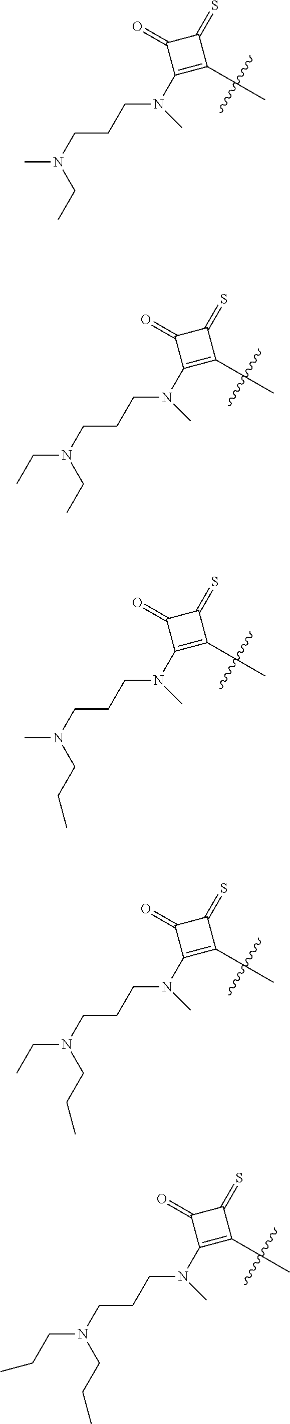 [SMILES: *c1c(N(C)CCCN(C)CC)c(=O)c1=S ; *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S]

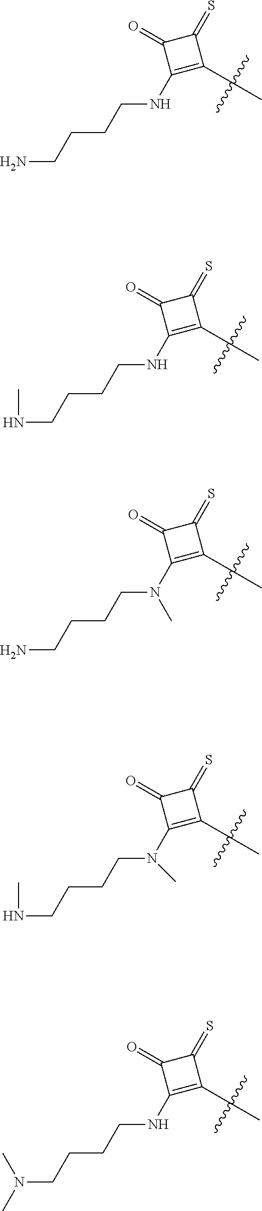 [SMILES: *c1c(NCCCCN)c(=O)c1=S ; *c1c(NCCCCNC)c(=O)c1=S ; *c1c(N(C)CCCCN)c(=O)c1=S ; *c1c(N(C)CCCCNC)c(=O)c1=S ; *c1c(NCCCCN(C)C)c(=O)c1=S]

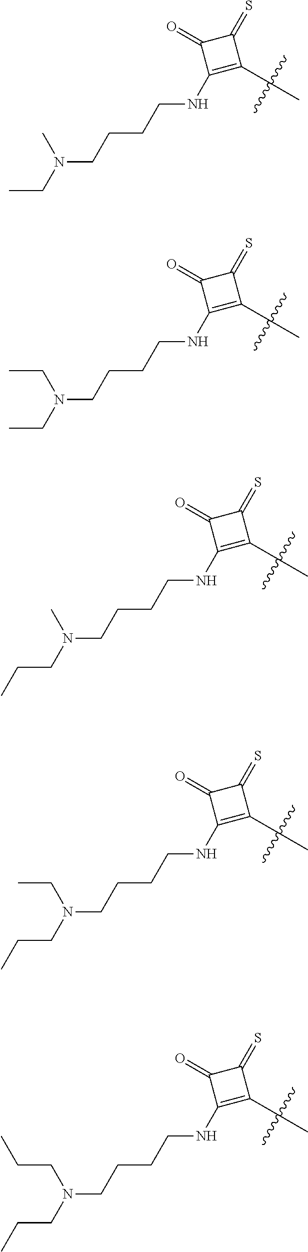 [SMILES: *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

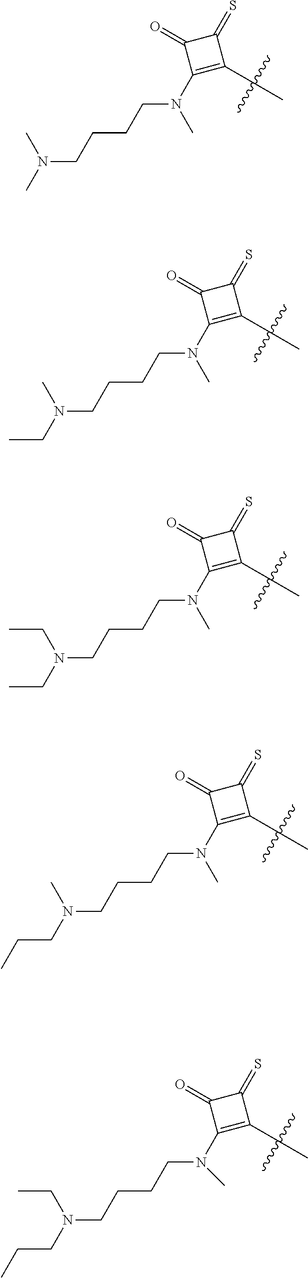 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S]

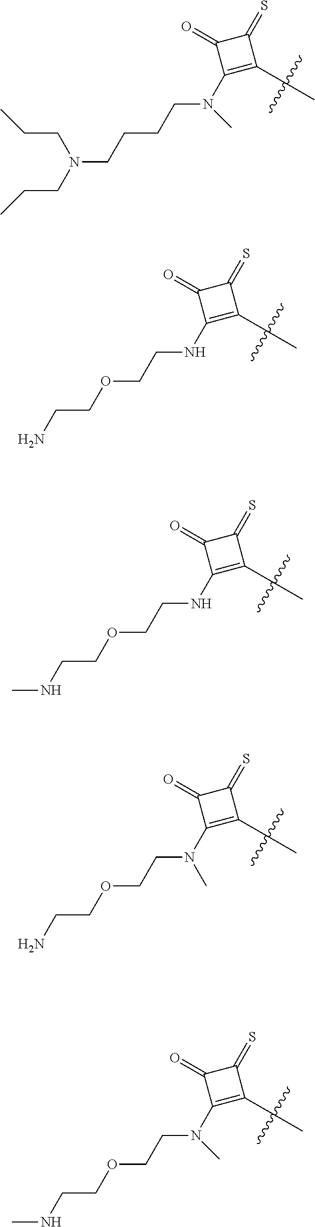 [SMILES: *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

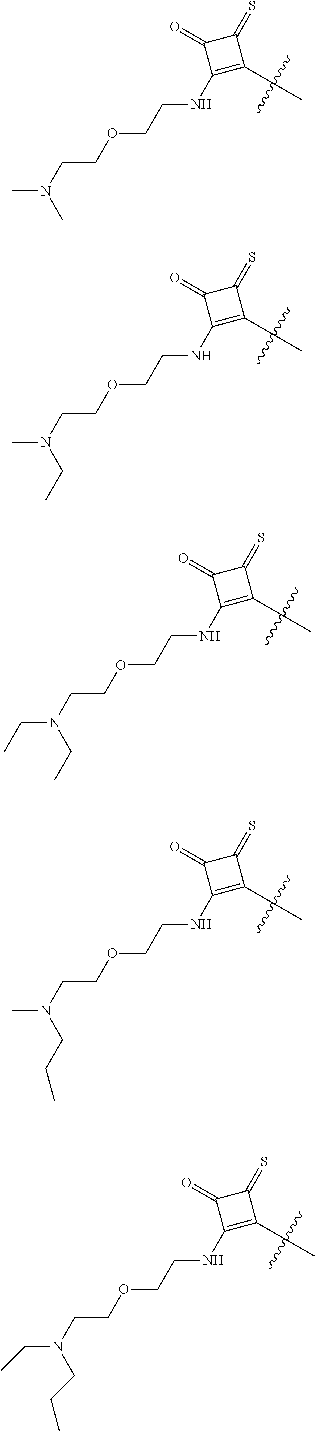 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

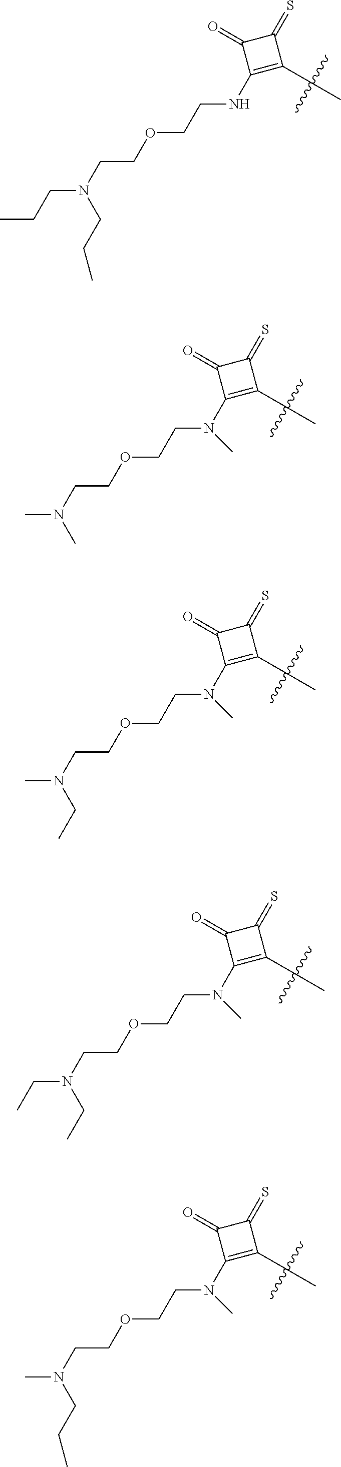 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

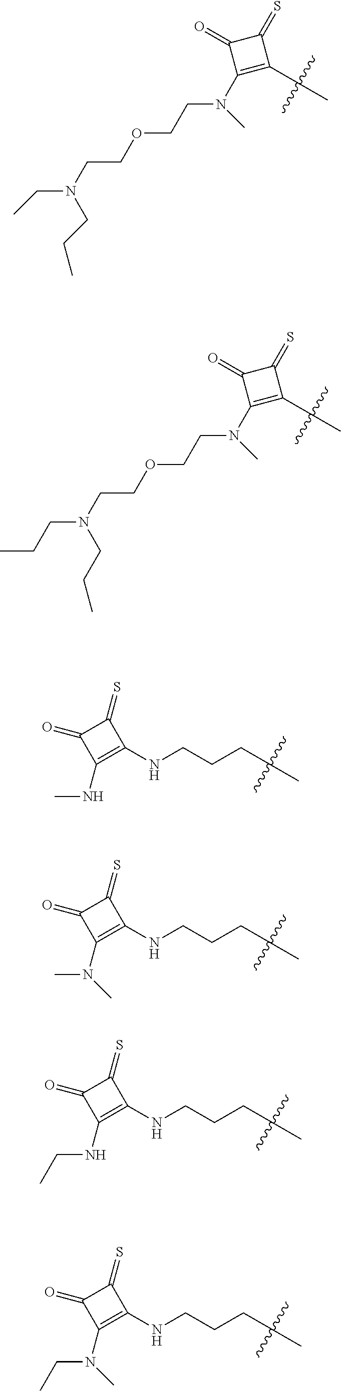 [SMILES: *c1c(N(C)CCOCCN(CC)CCC)c(=O)c1=S ; *c1c(N(C)CCOCCN(CCC)CCC)c(=O)c1=S ; *CCCNc1c(NC)c(=O)c1=S ; *CCCNc1c(N(C)C)c(=O)c1=S ; *CCCNc1c(NCC)c(=O)c1=S ; *CCCNc1c(N(C)CC)c(=O)c1=S]

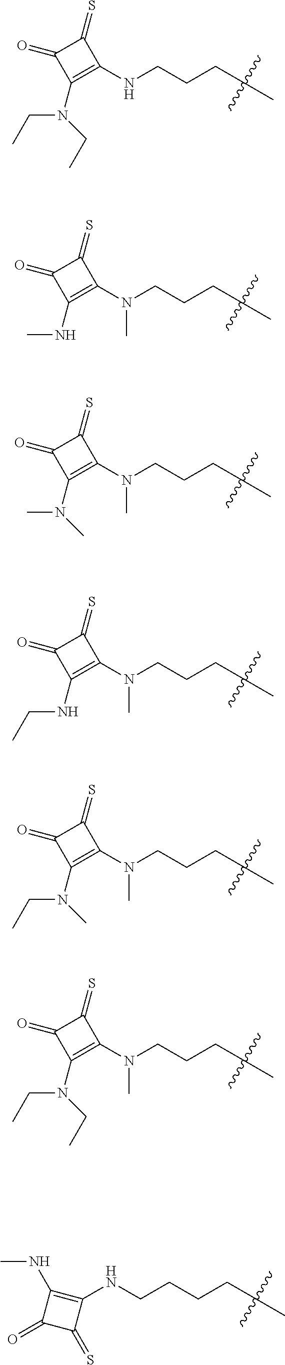

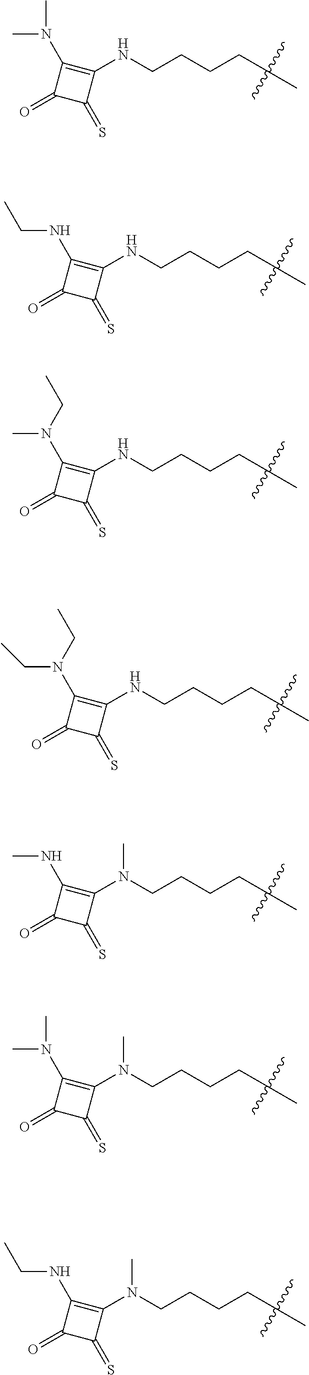

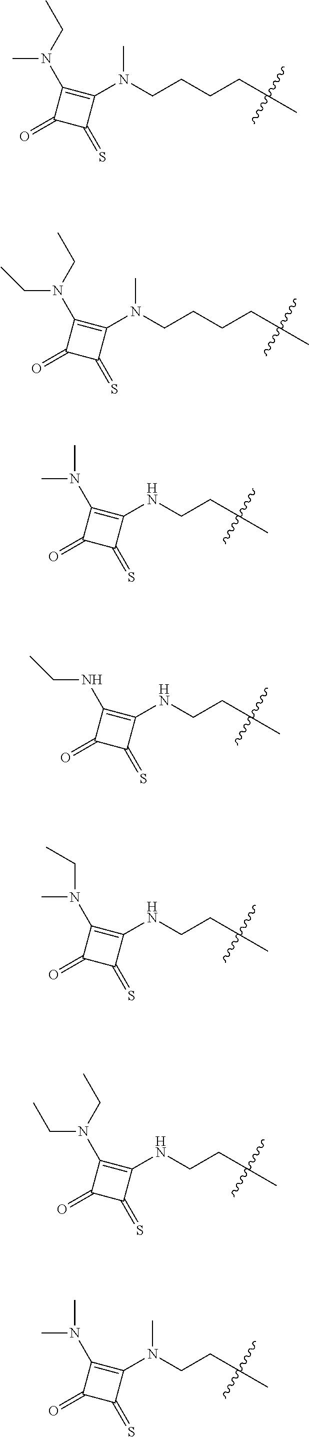

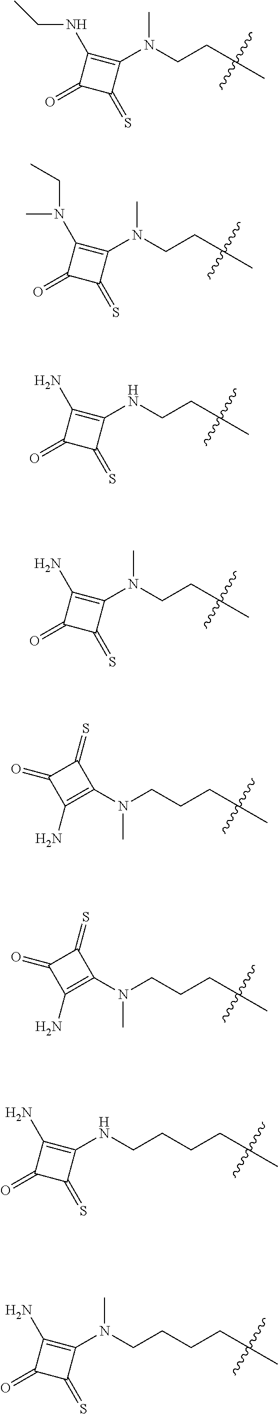

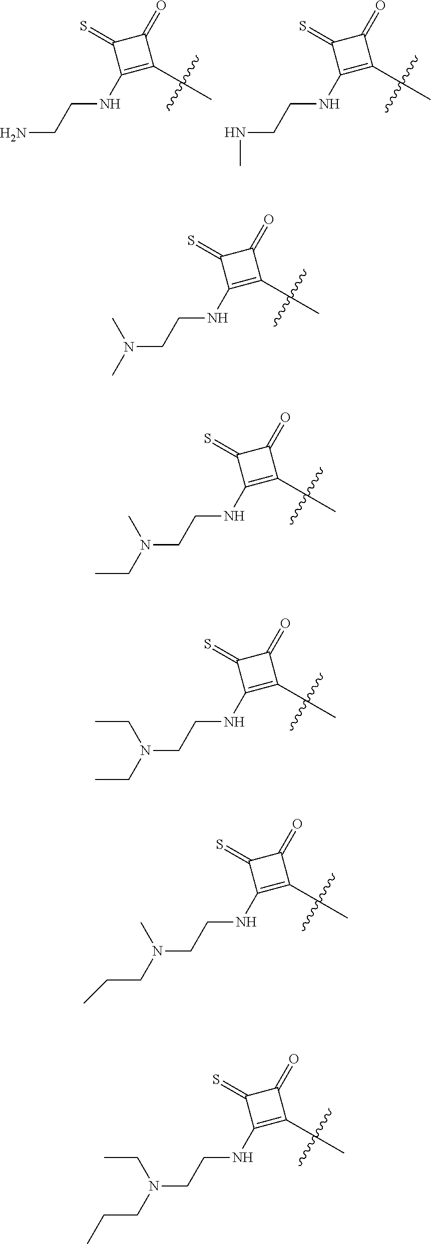

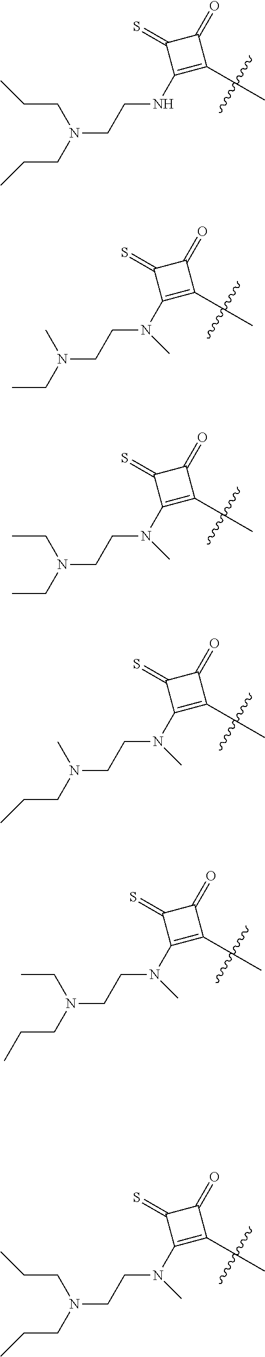 [SMILES: *c1c(NCCN(CCC)CCC)c(=S)c1=O ; *c1c(N(C)CCN(C)CC)c(=S)c1=O ; *c1c(N(C)CCN(CC)CC)c(=S)c1=O ; *c1c(N(C)CCN(C)CCC)c(=S)c1=O ; *c1c(N(C)CCN(CC)CCC)c(=S)c1=O ; *c1c(N(C)CCN(CCC)CCC)c(=S)c1=O]

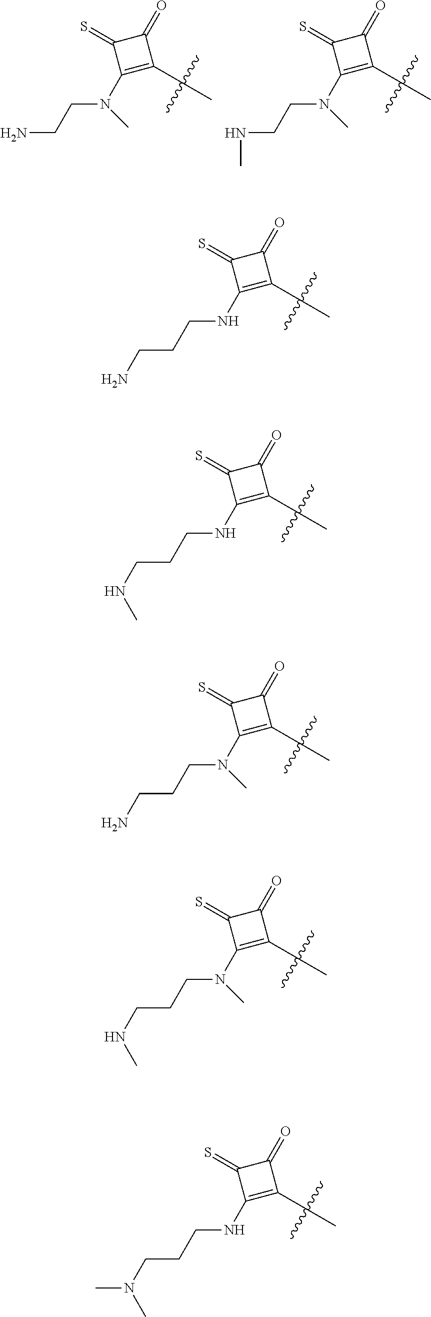

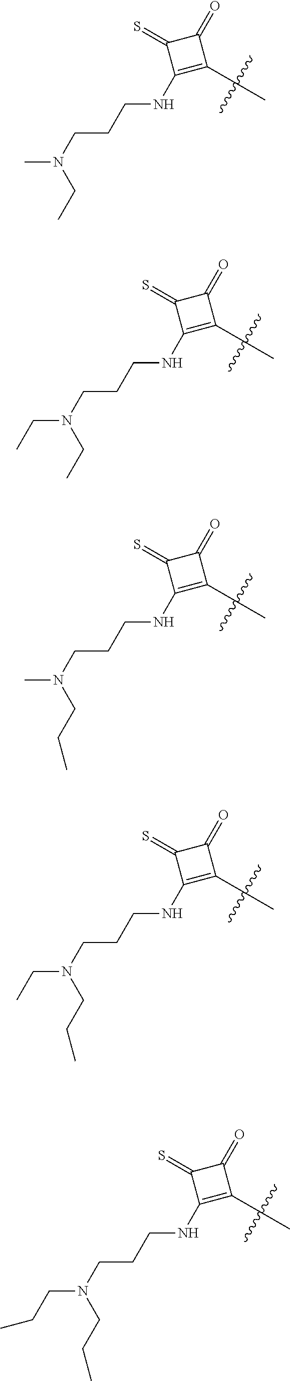 [SMILES: *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O ; *c1c(NCCCN(CCC)CCC)c(=S)c1=O]

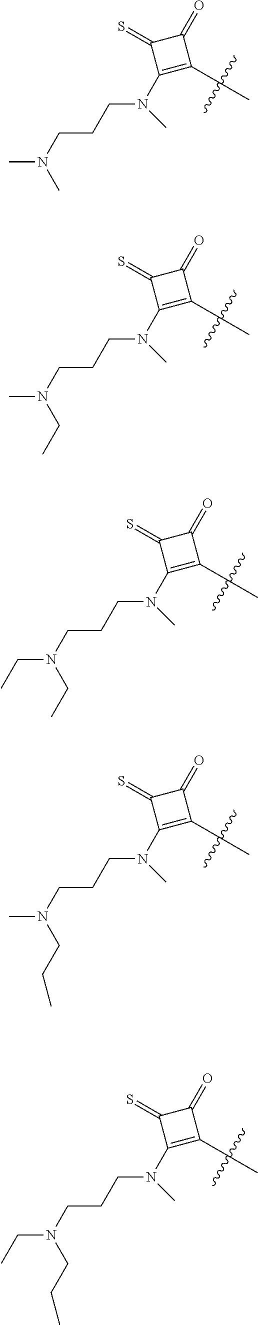 [SMILES: *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O]

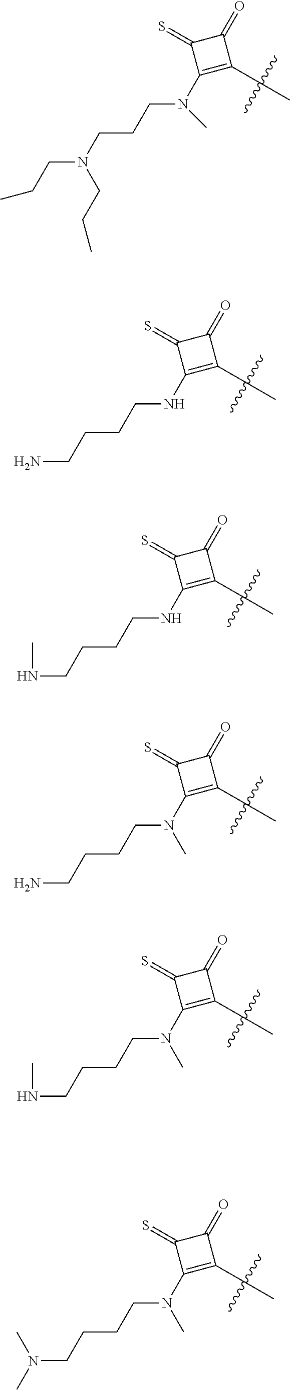 [SMILES: *c1c(N(C)CCCN(CCC)CCC)c(=S)c1=O ; *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O ; *c1c(N(C)CCCCN(C)C)c(=S)c1=O]

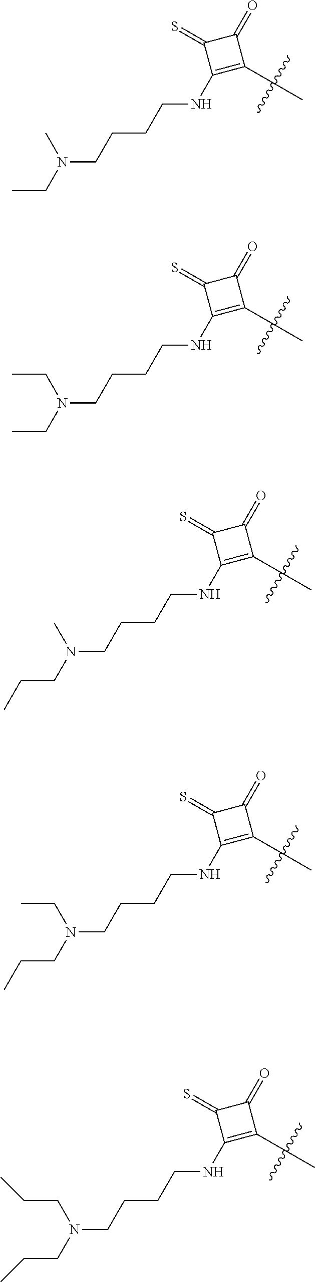 [SMILES: *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O]

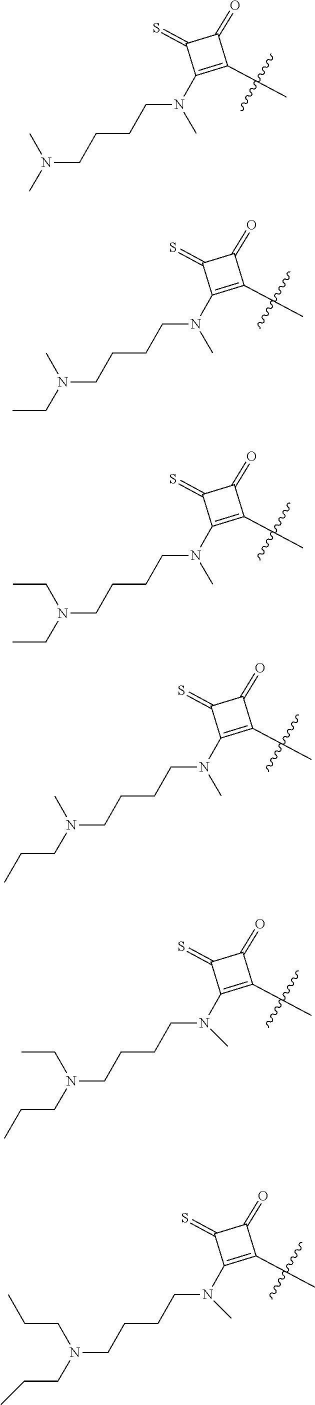 [SMILES: *c1c(N(C)CCCCN(C)C)c(=S)c1=O ; *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O]

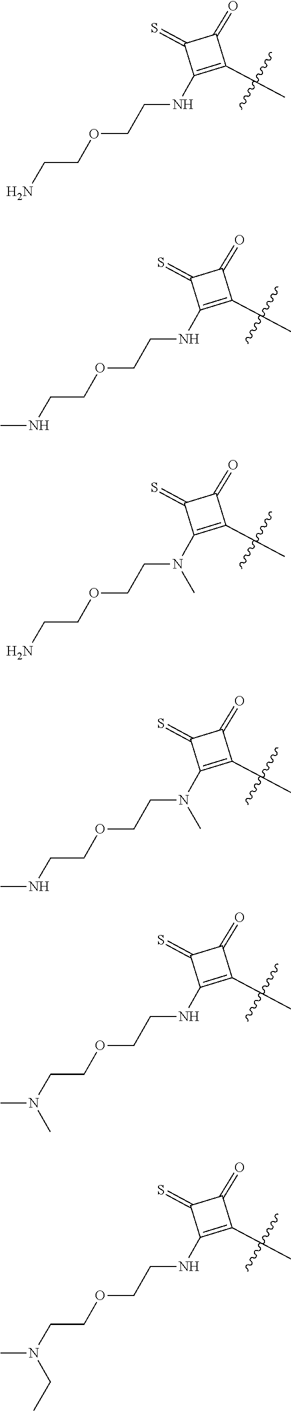 [SMILES: *c1c(NCCOCCN)c(=S)c1=O ; *c1c(NCCOCCNC)c(=S)c1=O ; *c1c(N(C)CCOCCN)c(=S)c1=O ; *c1c(N(C)CCOCCNC)c(=S)c1=O ; *c1c(NCCOCCN(C)C)c(=S)c1=O ; *c1c(NCCOCCN(C)CC)c(=S)c1=O]

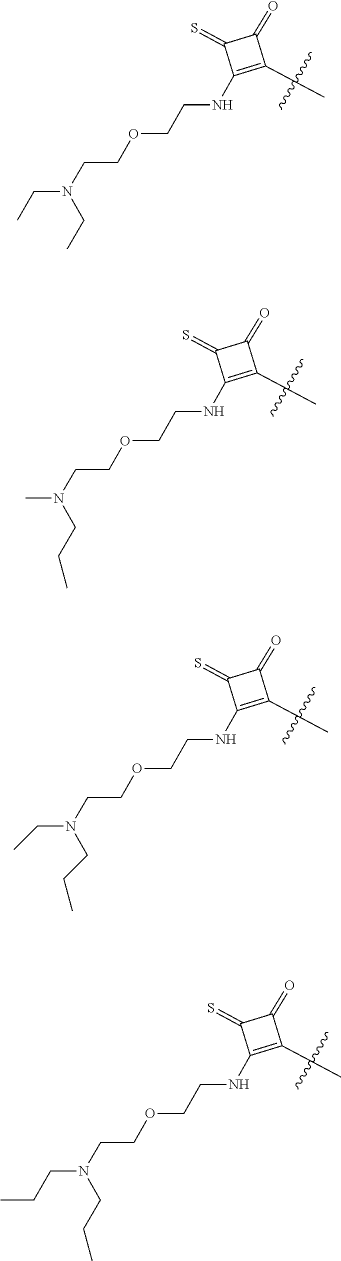 [SMILES: *c1c(NCCOCCN(CC)CC)c(=S)c1=O ; *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O]

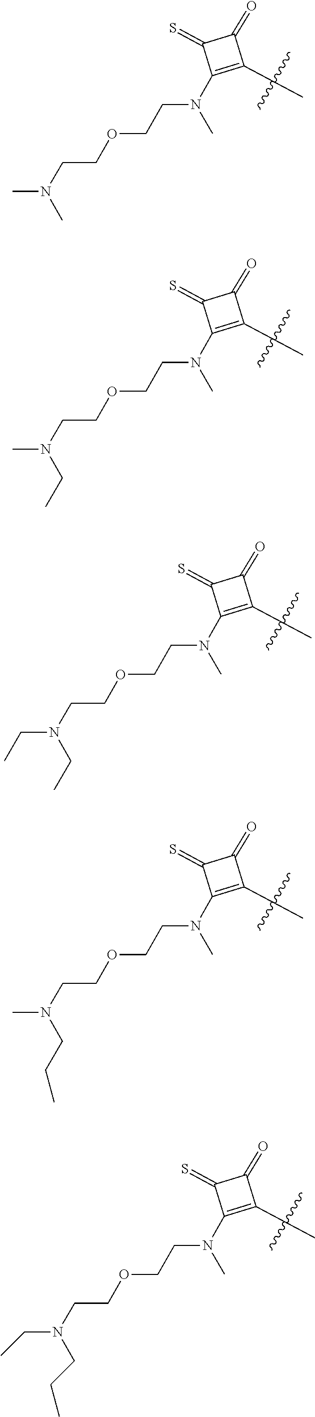 [SMILES: *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O]

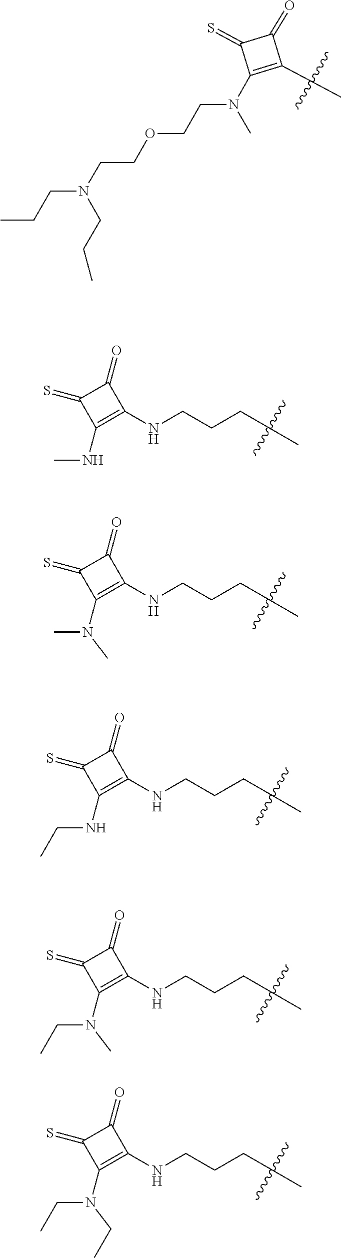

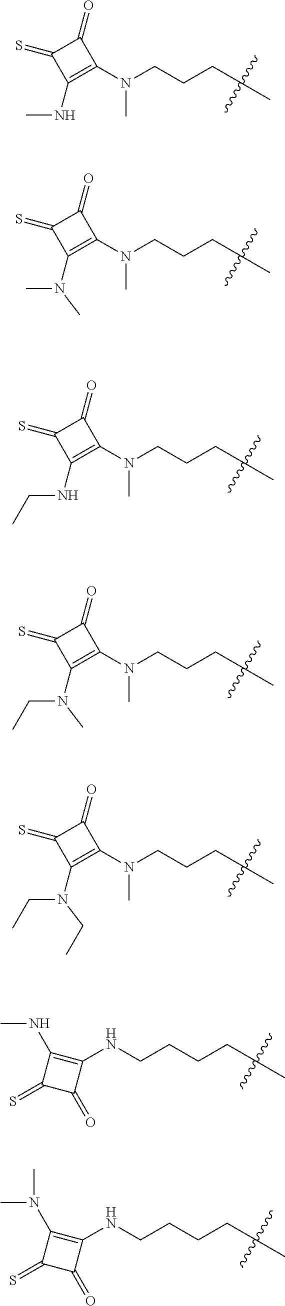

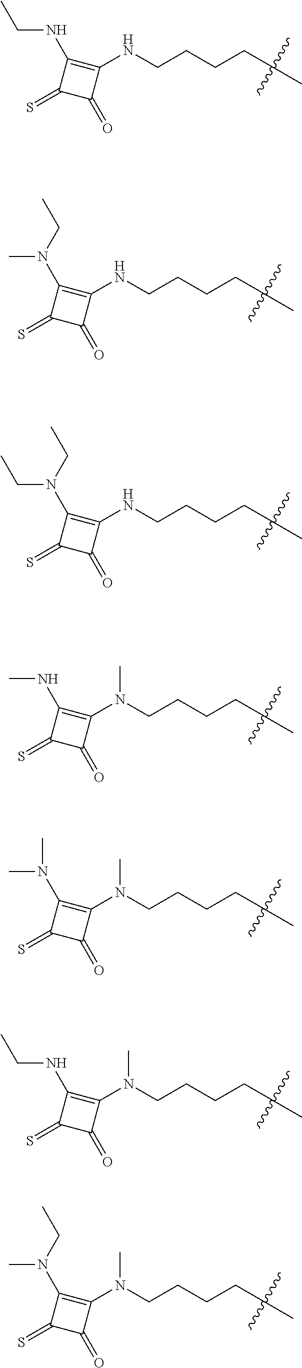

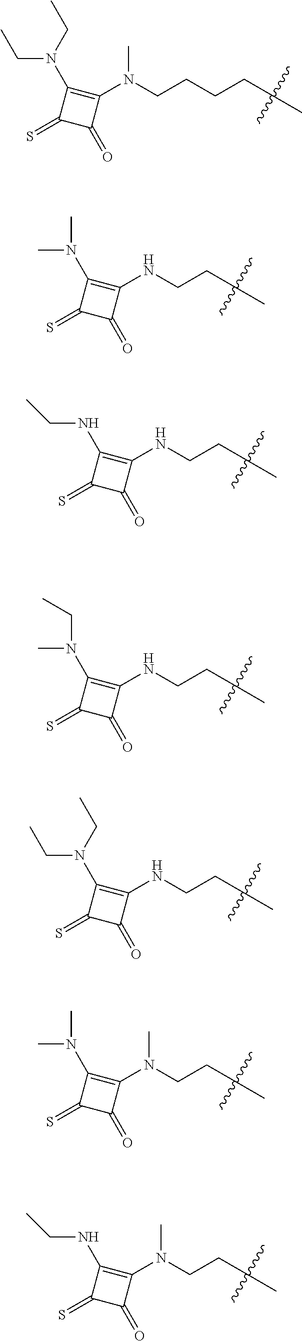

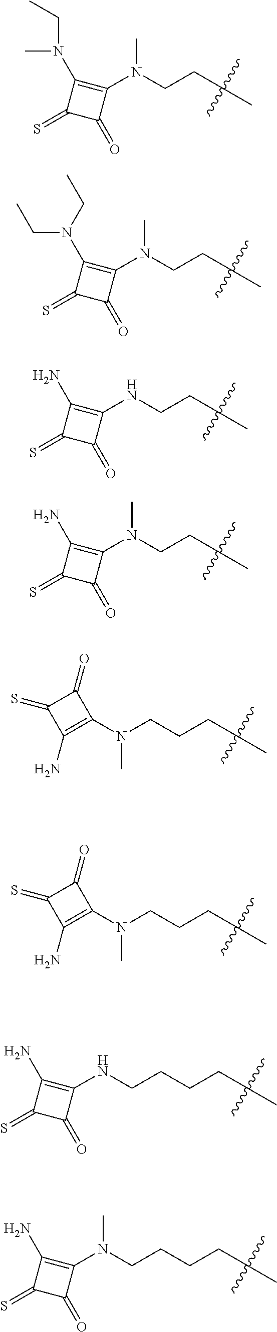

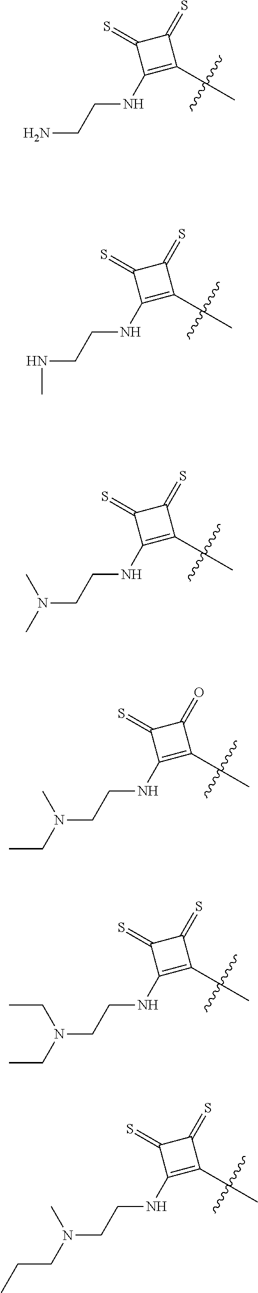 [SMILES: *c1c(NCCN)c(=S)c1=S ; *c1c(NCCNC)c(=S)c1=S ; *c1c(NCCN(C)C)c(=S)c1=S ; *c1c(NCCN(C)CC)c(=S)c1=O ; *c1c(NCCN(CC)CC)c(=S)c1=S ; *c1c(NCCN(C)CCC)c(=S)c1=S]

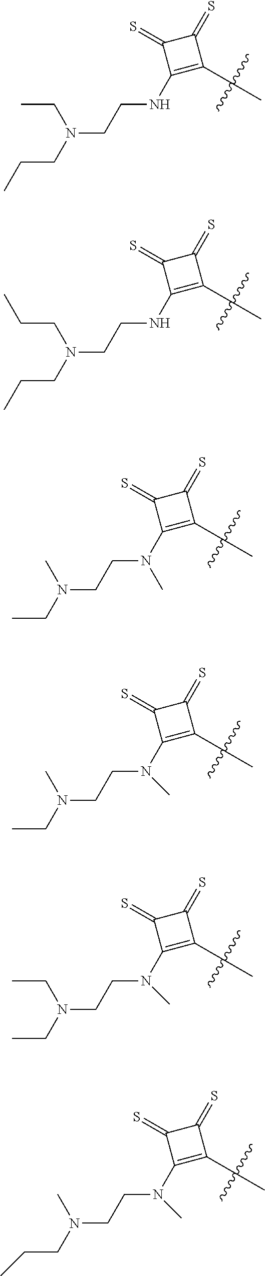 [SMILES: *c1c(NCCN(CC)CCC)c(=S)c1=S ; *c1c(NCCN(CCC)CCC)c(=S)c1=S ; *c1c(N(C)CCN(C)CC)c(=S)c1=S ; *c1c(N(C)CCN(C)CC)c(=S)c1=S ; *c1c(N(C)CCN(CC)CC)c(=S)c1=S ; *c1c(N(C)CCN(C)CCC)c(=S)c1=S]

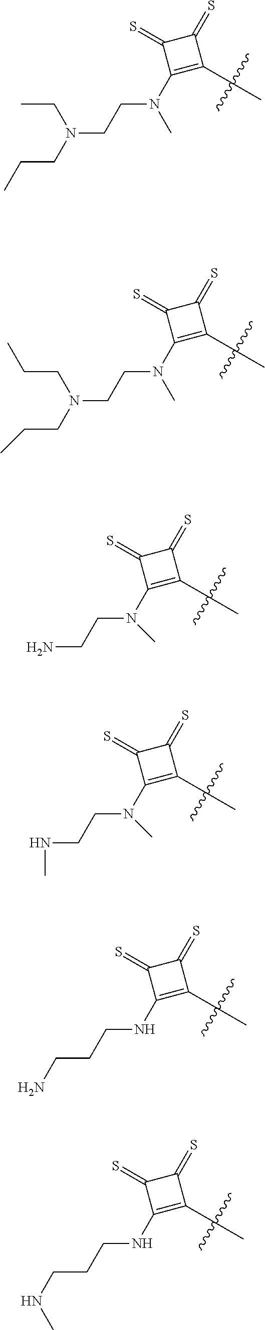 [SMILES: *c1c(N(C)CCN(CC)CCC)c(=S)c1=S ; *c1c(N(C)CCN(CCC)CCC)c(=S)c1=S ; *c1c(N(C)CCN)c(=S)c1=S ; *c1c(N(C)CCNC)c(=S)c1=S ; *c1c(NCCCN)c(=S)c1=S ; *c1c(NCCCNC)c(=S)c1=S]

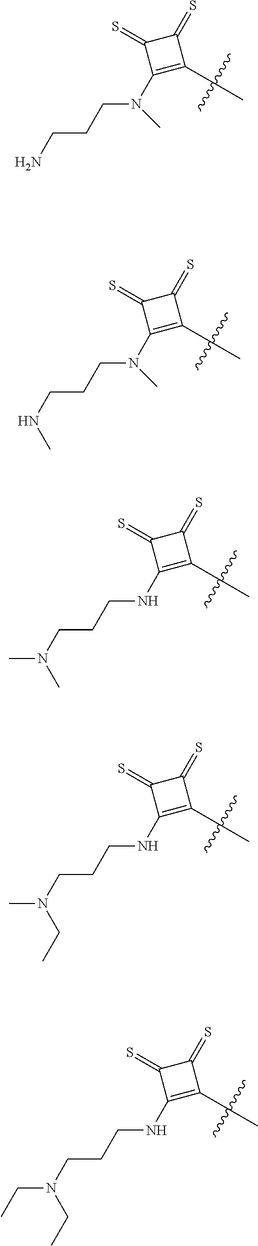 [SMILES: *c1c(N(C)CCCN)c(=S)c1=S ; *c1c(N(C)CCCNC)c(=S)c1=S ; *c1c(NCCCN(C)C)c(=S)c1=S ; *c1c(NCCCN(C)CC)c(=S)c1=S ; *c1c(NCCCN(CC)CC)c(=S)c1=S]

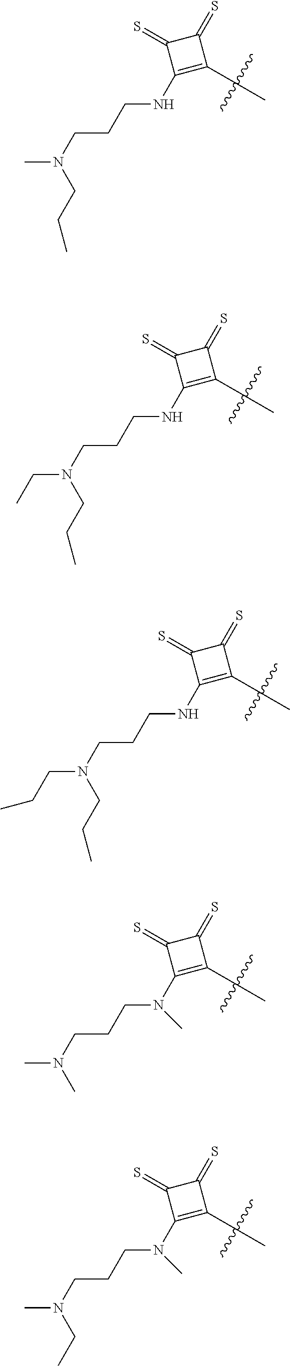 [SMILES: *c1c(NCCCN(C)CCC)c(=S)c1=S ; *c1c(NCCCN(CC)CCC)c(=S)c1=S ; *c1c(NCCCN(CCC)CCC)c(=S)c1=S ; *c1c(N(C)CCCN(C)C)c(=S)c1=S ; *c1c(N(C)CCCN(C)CC)c(=S)c1=S]

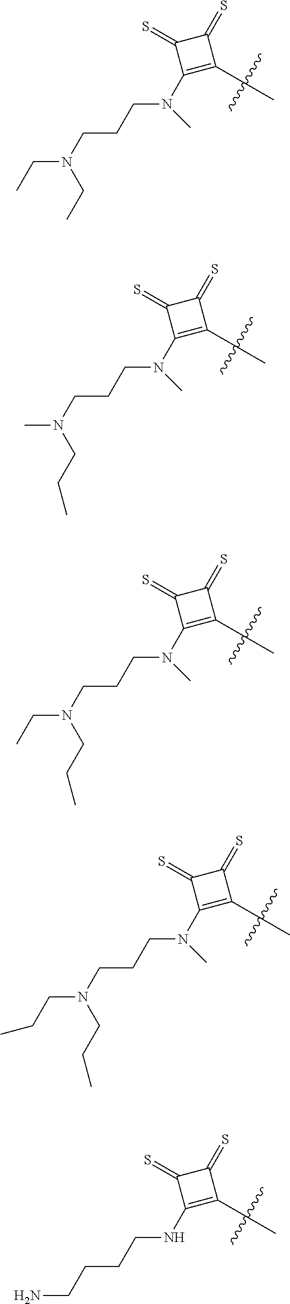 [SMILES: *c1c(N(C)CCCN(CC)CC)c(=S)c1=S ; *c1c(N(C)CCCN(C)CCC)c(=S)c1=S ; *c1c(N(C)CCCN(CC)CCC)c(=S)c1=S ; *c1c(N(C)CCCN(CCC)CCC)c(=S)c1=S ; *c1c(NCCCCN)c(=S)c1=S]

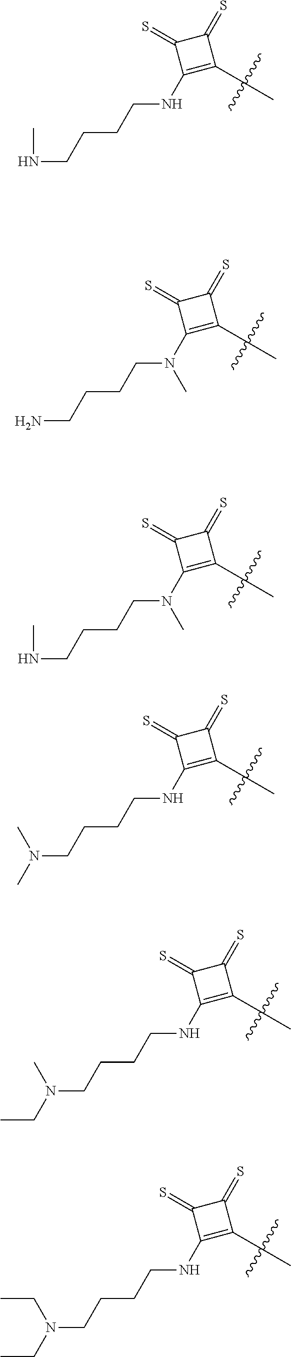 [SMILES: *c1c(NCCCCNC)c(=S)c1=S ; *c1c(N(C)CCCCN)c(=S)c1=S ; *c1c(N(C)CCCCNC)c(=S)c1=S ; *c1c(NCCCCN(C)C)c(=S)c1=S ; *c1c(NCCCCN(C)CC)c(=S)c1=S ; *c1c(NCCCCN(CC)CC)c(=S)c1=S]

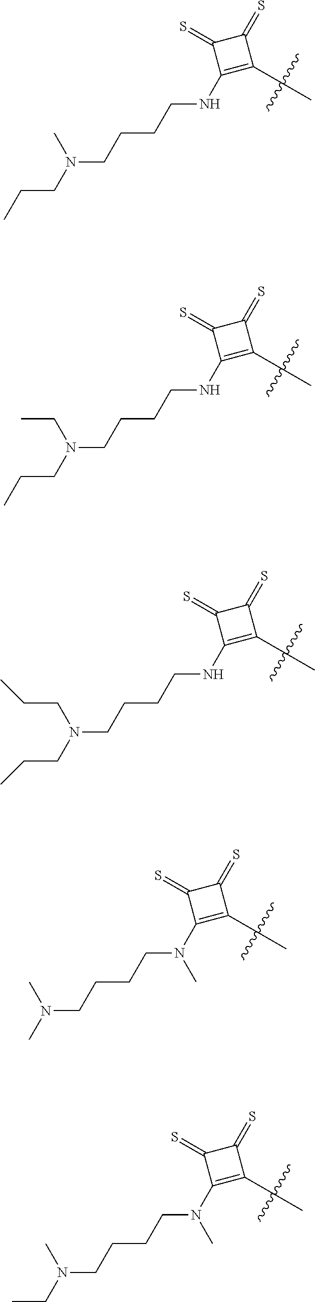 [SMILES: *c1c(NCCCCN(C)CCC)c(=S)c1=S ; *c1c(NCCCCN(CC)CCC)c(=S)c1=S ; *c1c(NCCCCN(CCC)CCC)c(=S)c1=S ; *c1c(N(C)CCCCN(C)C)c(=S)c1=S ; *c1c(N(C)CCCCN(C)CC)c(=S)c1=S]

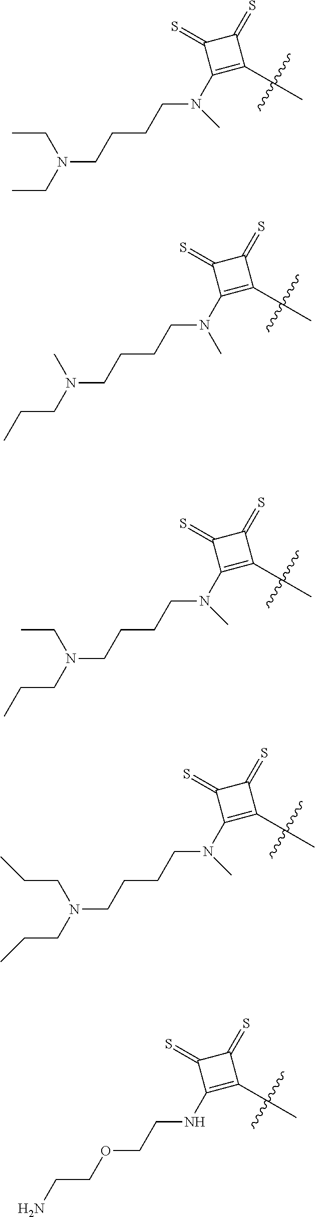 [SMILES: *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S ; *c1c(N(C)CCCCN(CCC)CCC)c(=S)c1=S ; *c1c(NCCOCCN)c(=S)c1=S]

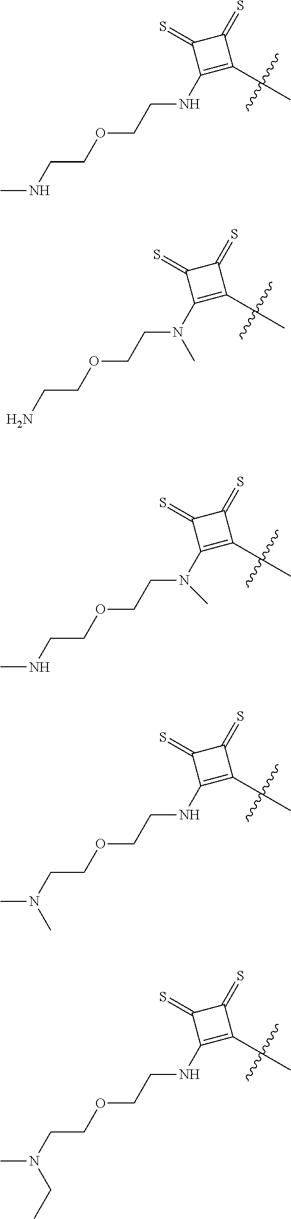 [SMILES: *c1c(NCCOCCNC)c(=S)c1=S ; *c1c(N(C)CCOCCN)c(=S)c1=S ; *c1c(N(C)CCOCCNC)c(=S)c1=S ; *c1c(NCCOCCN(C)C)c(=S)c1=S ; *c1c(NCCOCCN(C)CC)c(=S)c1=S]

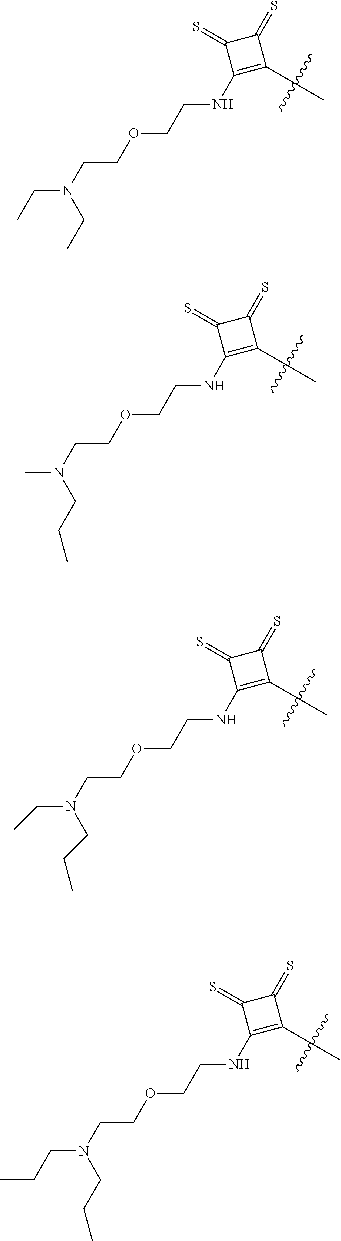 [SMILES: *c1c(NCCOCCN(CC)CC)c(=S)c1=S ; *c1c(NCCOCCN(C)CCC)c(=S)c1=S ; *c1c(NCCOCCN(CC)CCC)c(=S)c1=S ; *c1c(NCCOCCN(CCC)CCC)c(=S)c1=S]

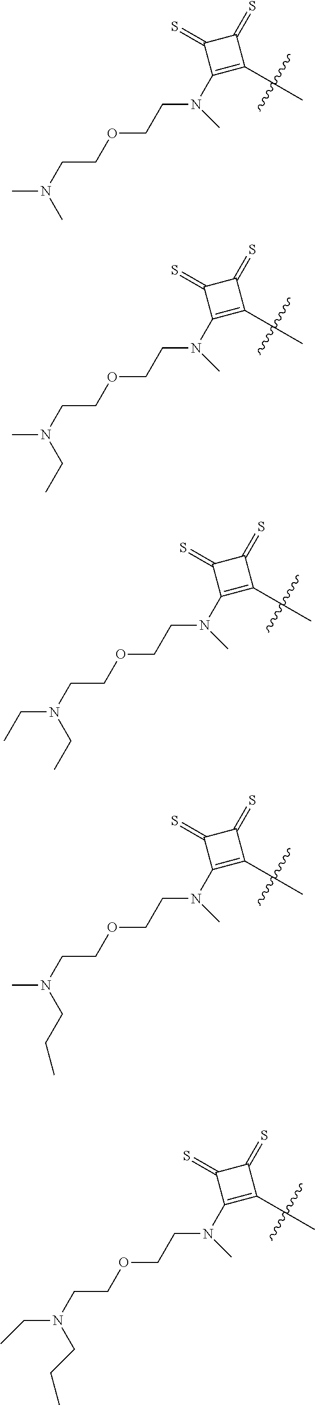 [SMILES: *c1c(N(C)CCOCCN(C)C)c(=S)c1=S ; *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S]

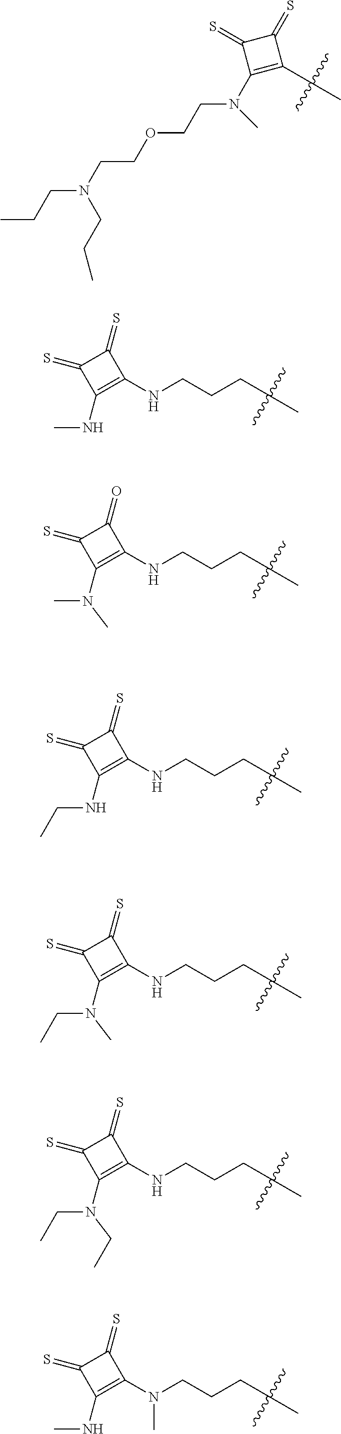

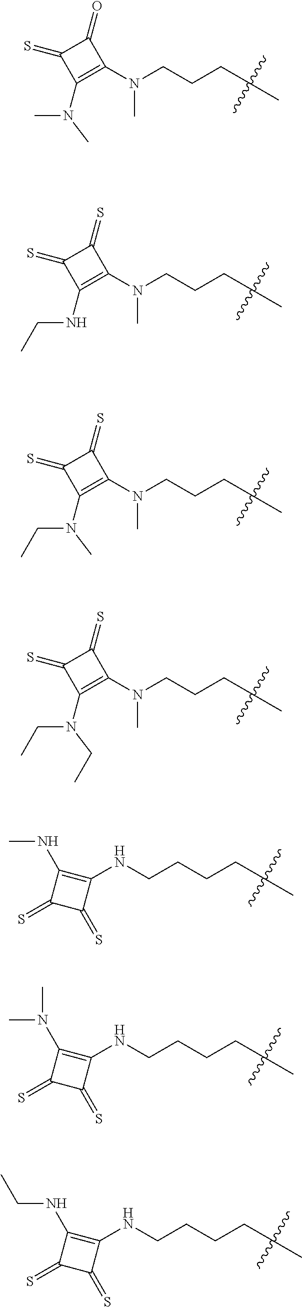

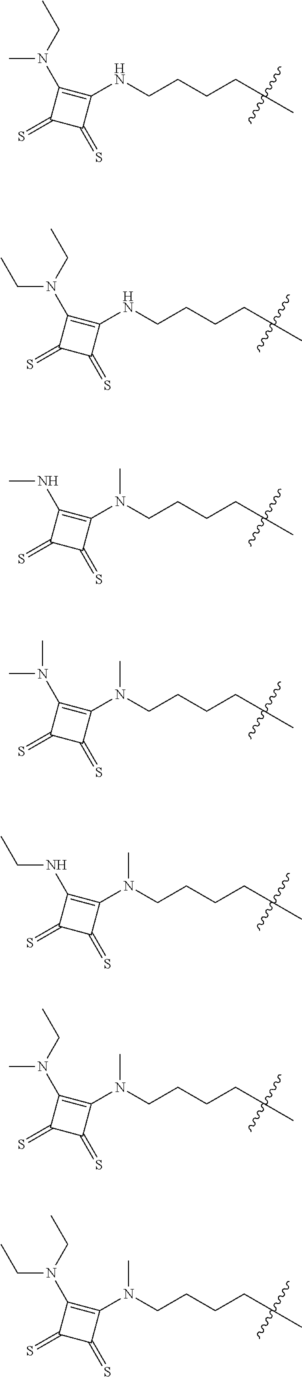

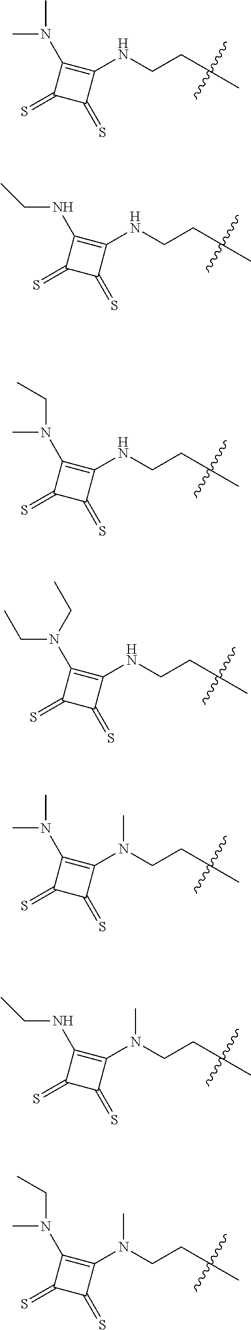

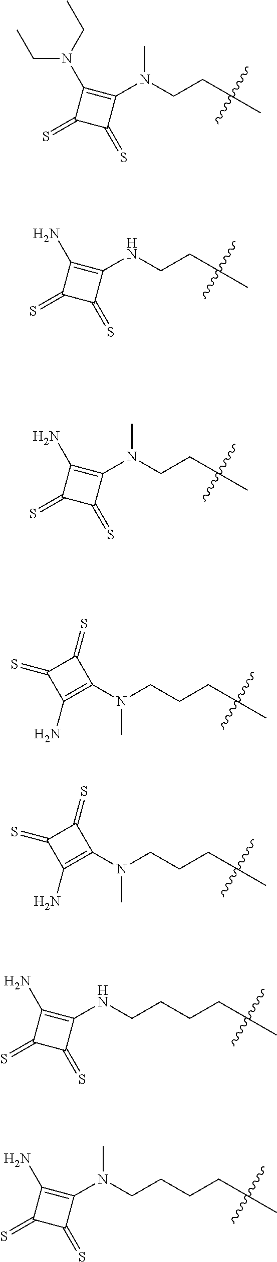

In some embodiments, W is selected from an of the following groups:

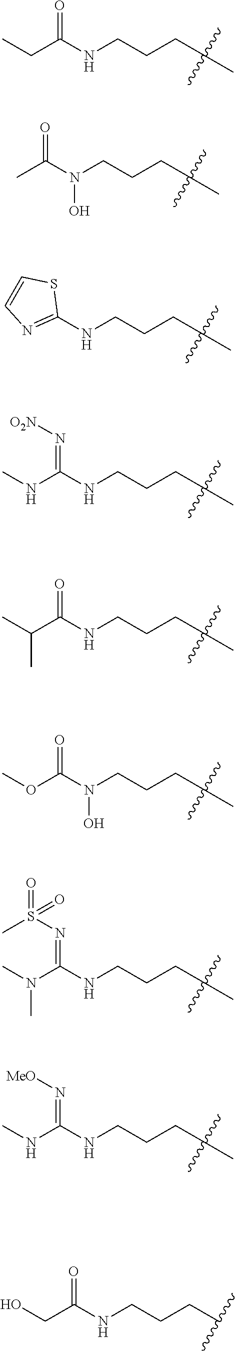

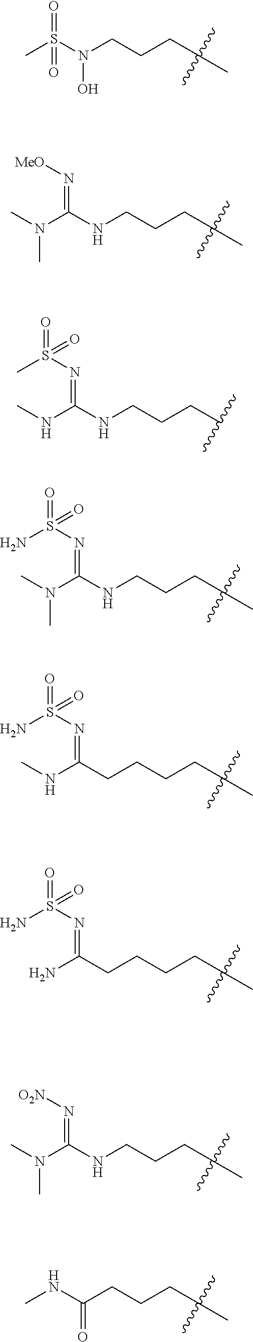

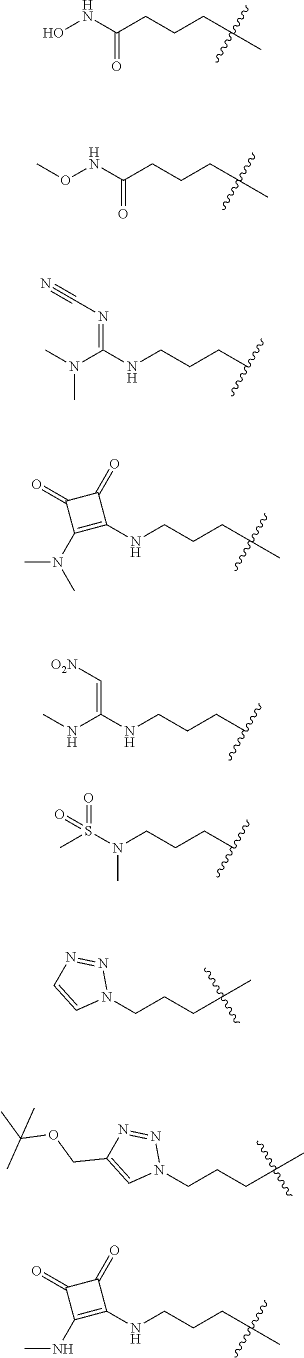

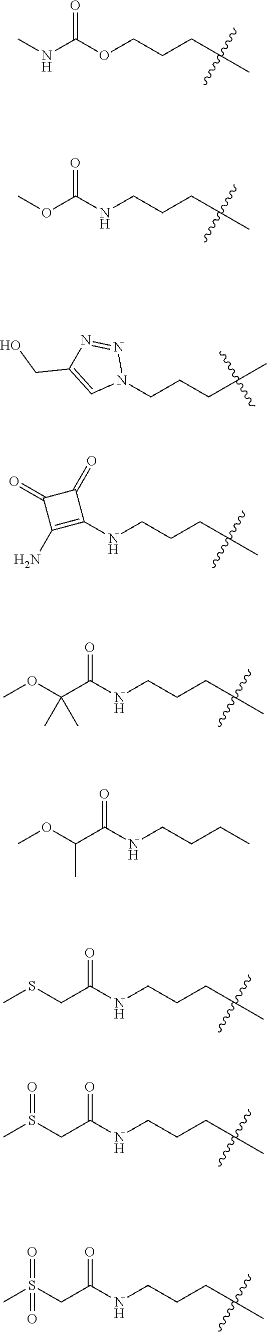

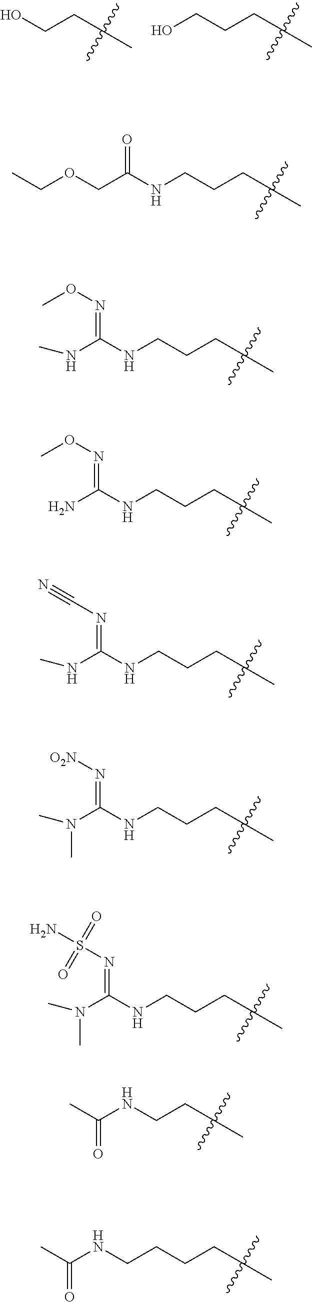

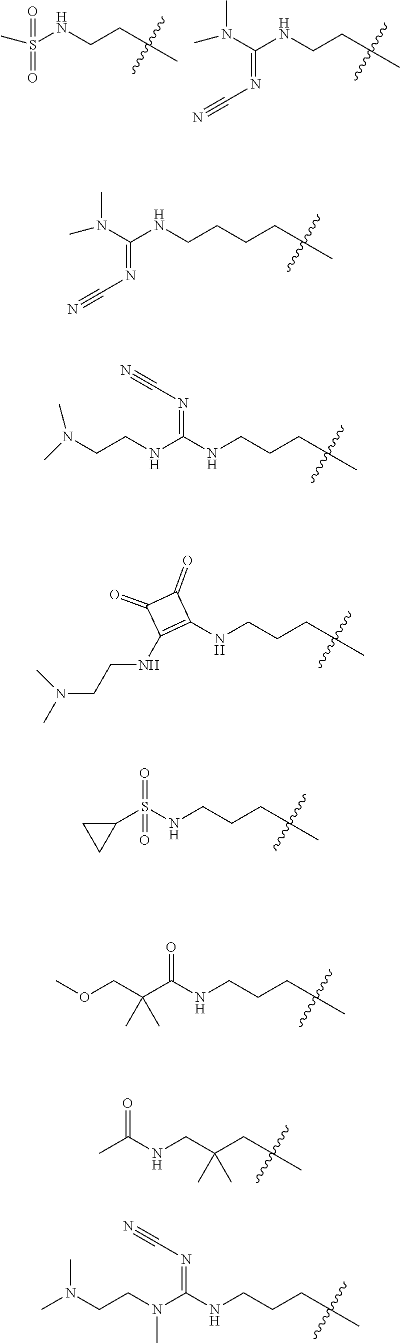

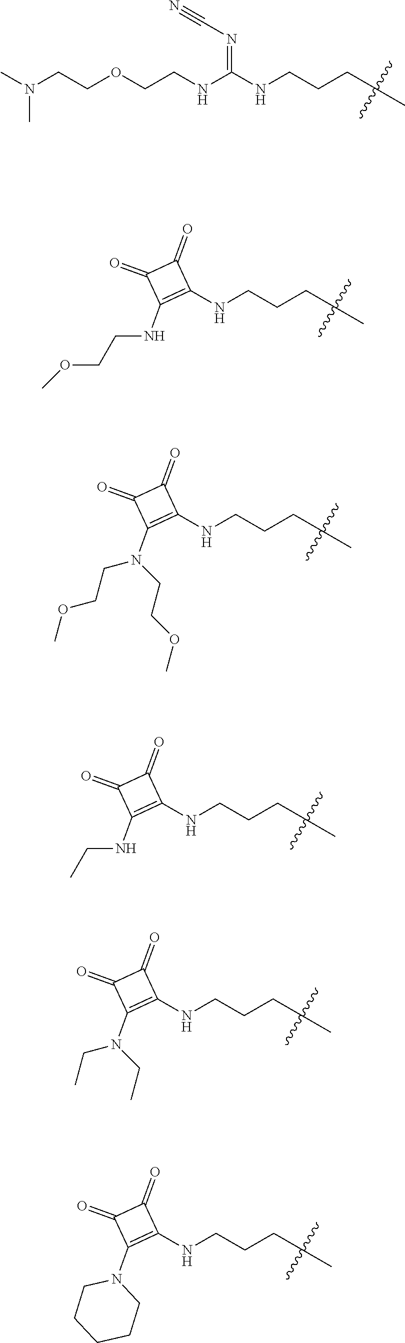 [SMILES: *CCCN/C(=N/C#N)NCCOCCN(C)C ; *CCCNc1c(NCCOC)c(=O)c1=O ; *CCCNc1c(N(CCOC)CCOC)c(=O)c1=O ; *CCCNc1c(NCC)c(=O)c1=O ; *CCCNc1c(N(CC)CC)c(=O)c1=O ; *CCCNc1c(N2CCCCC2)c(=O)c1=O]

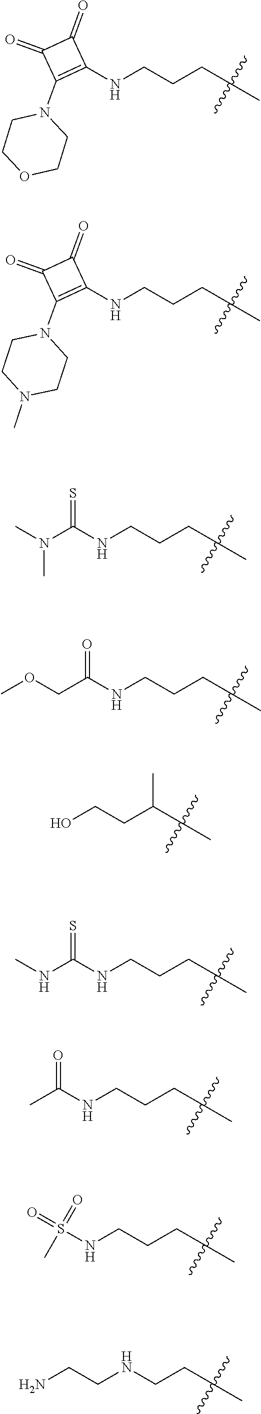

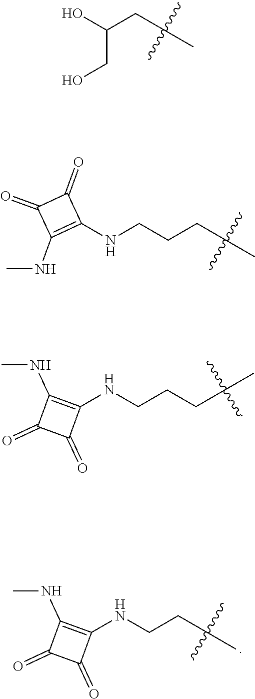 [SMILES: *CC(O)CO ; *CCCNc1c(NC)c(=O)c1=O ; *CCCNc1c(NC)c(=O)c1=O ; *CCNc1c(NC)c(=O)c1=O]

In some embodiments,

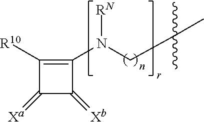

is selected from any of the following groups:

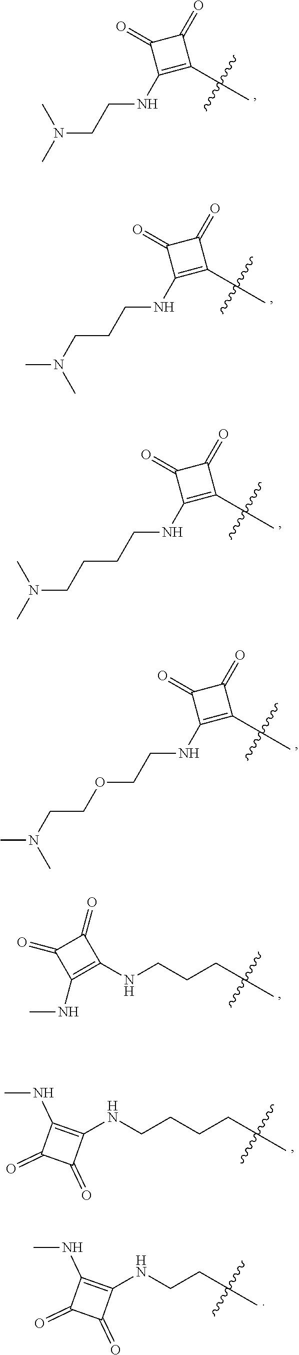

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration. In some embodiments the compound of any of the formulae described herein is suitable for making a nanoparticle composition for subcutaneous administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R₈, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle.

In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments, R^{N} is C₁ alkyl. For example, in some embodiments, R^{N} is C₂ alkyl. For example, in some embodiments, R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)₁₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is-NH(CH₂)ₒN(R)₂, o is 2, 3, or 4.

In some embodiments wherein —NH(CH₂)ₚ₁O(CH₂)_{q1}N(R)₂, p1 is 2. In some embodiments wherein —NH(CH₂)ₚ₁O(CH₂)_{q1}N(R)₂, q1 is 2.

In some embodiments wherein R¹⁰ is —N((CH₂)ₛ₁OR)₂, s1 is 2.

In some embodiments wherein R¹⁰ is-NH(CH₂)ₒN(R)₂, —NH(CH₂)ₚO(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is-NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methyhlpiperazinyl.

In some embodiments, each occurrence of R⁵ and R⁶ is H. In some embodiments, the compound of Formula (I) is selected from the group consisting of:

| Cpd | Structure |
|---|---|
| I1 | 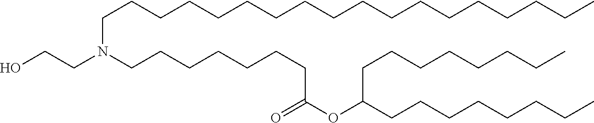 [SMILES: CCCCCCCCCCCCCCCCCCN(CCO)CCCCCCCC(=O)OC(CCCCCCCC)CCCCCCCC] |
| I2 | 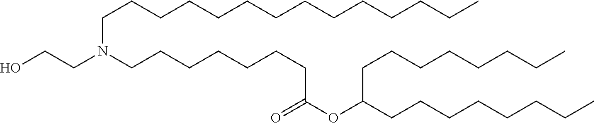 [SMILES: CCCCCCCCCCCCCCN(CCO)CCCCCCCC(=O)OC(CCCCCCCC)CCCCCCCC] |
| I3 | 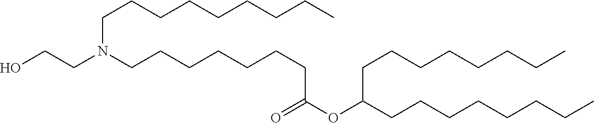 [SMILES: CCCCCCCCCN(CCO)CCCCCCCC(=O)OC(CCCCCCCC)CCCCCCCC] |
| I4 | 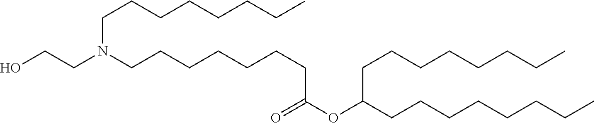 [SMILES: CCCCCCCCC(CCCCCCCC)OC(=O)CCCCCCCN(CCO)CCCCCCCC] |
| I5 | 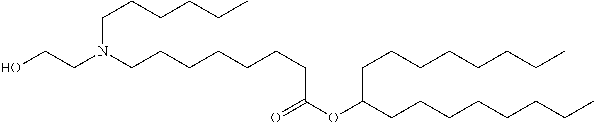 [SMILES: CCCCCCCCC(CCCCCCCC)OC(=O)CCCCCCCN(CCO)CCCCCC] |
| I6 | 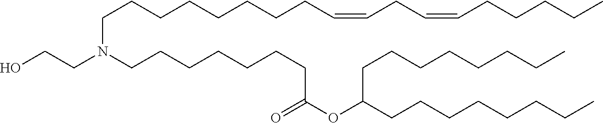 [SMILES: CCCCC/C=C\C/C=C\CCCCCCCCN(CCO)CCCCCCCC(=O)OC(CCCCCCCC)CCCCCCCC] |
| I7 | 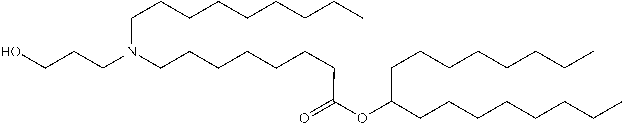 [SMILES: CCCCCCCCCN(CCCO)CCCCCCCC(=O)OC(CCCCCCCC)CCCCCCCC] |
| I8 | 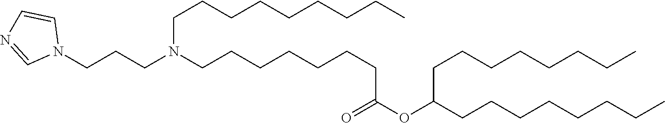 [SMILES: CCCCCCCCCN(CCCCCCCC(=O)OC(CCCCCCCC)CCCCCCCC)CCCn1ccnc1] |
| I9 | 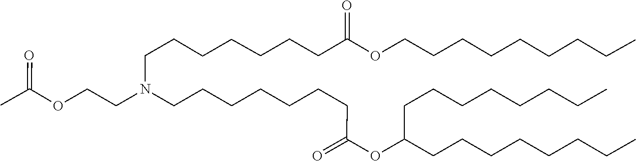 [SMILES: CCCCCCCCCOC(=O)CCCCCCCN(CCCCCCCC(=O)OC(CCCCCCCC)CCCCCCCC)CCOC(C)=O] |
| I10 | 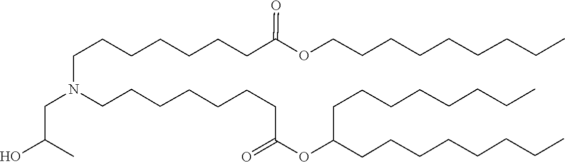 [SMILES: CCCCCCCCCOC(=O)CCCCCCCN(CCCCCCCC(=O)OC(CCCCCCCC)CCCCCCCC)CC(C)O] |
| I11 | 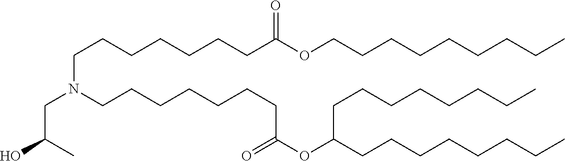 [SMILES: CCCCCCCCCOC(=O)CCCCCCCN(CCCCCCCC(=O)OC(CCCCCCCC)CCCCCCCC)C[C@@H](C)O] |
| I12 | 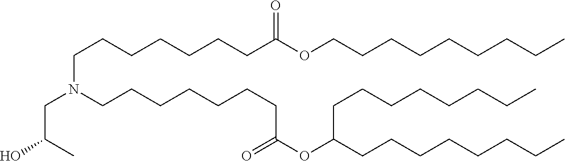 [SMILES: CCCCCCCCCOC(=O)CCCCCCCN(CCCCCCCC(=O)OC(CCCCCCCC)CCCCCCCC)C[C@H](C)O] |
| I13 | 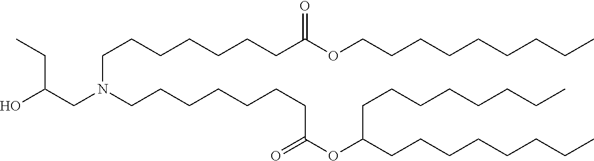 [SMILES: CCCCCCCCCOC(=O)CCCCCCCN(CCCCCCCC(=O)OC(CCCCCCCC)CCCCCCCC)CC(O)CC] |
| I14 | 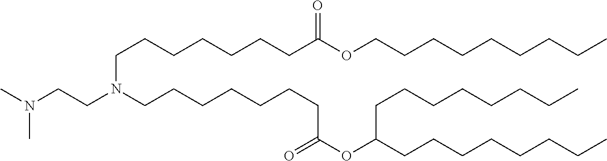 [SMILES: CCCCCCCCCOC(=O)CCCCCCCN(CCCCCCCC(=O)OC(CCCCCCCC)CCCCCCCC)CCN(C)C] |
| I15 | 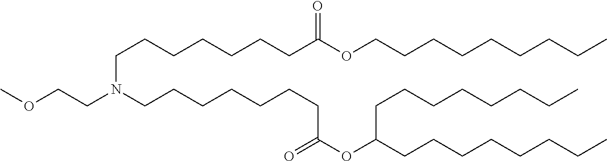 [SMILES: CCCCCCCCCOC(=O)CCCCCCCN(CCCCCCCC(=O)OC(CCCCCCCC)CCCCCCCC)CCOC] |
| I16 | 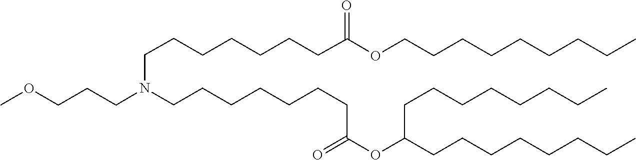 [SMILES: CCCCCCCCCOC(=O)CCCCCCCN(CCCCCCCC(=O)OC(CCCCCCCC)CCCCCCCC)CCCOC] |
| I17 | 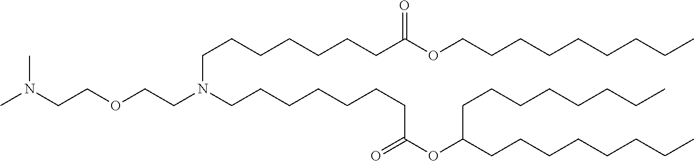 [SMILES: CCCCCCCCCOC(=O)CCCCCCCN(CCCCCCCC(=O)OC(CCCCCCCC)CCCCCCCC)CCOCCN(C)C] |
| I18 | 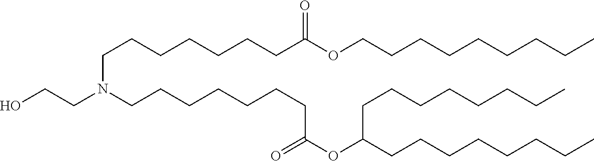 [SMILES: CCCCCCCCCOC(=O)CCCCCCCN(CCO)CCCCCCCC(=O)OC(CCCCCCCC)CCCCCCCC] |
| I19 | 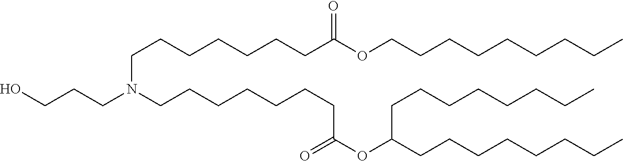 [SMILES: CCCCCCCCCOC(=O)CCCCCCCN(CCCO)CCCCCCCC(=O)OC(CCCCCCCC)CCCCCCCC] |
| I20 | 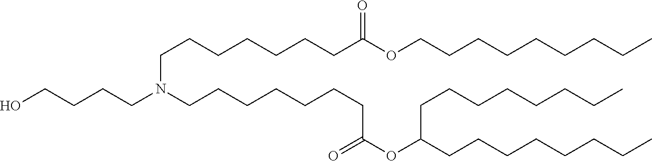 [SMILES: CCCCCCCCCOC(=O)CCCCCCCN(CCCCO)CCCCCCCC(=O)OC(CCCCCCCC)CCCCCCCC] |
| I21 | 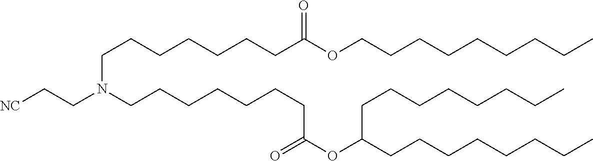 [SMILES: CCCCCCCCCOC(=O)CCCCCCCN(CCC#N)CCCCCCCC(=O)OC(CCCCCCCC)CCCCCCCC] |
| I22 | 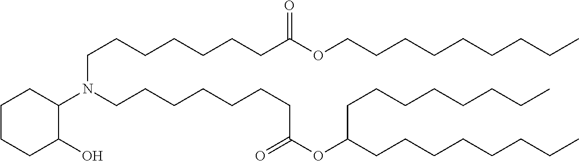 [SMILES: CCCCCCCCCOC(=O)CCCCCCCN(CCCCCCCC(=O)OC(CCCCCCCC)CCCCCCCC)C1CCCCC1O] |
| I23 | 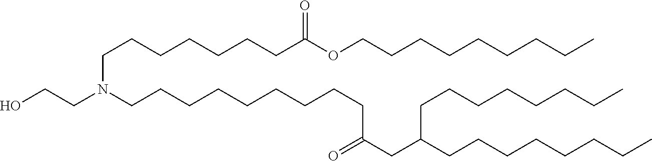 [SMILES: CCCCCCCCCOC(=O)CCCCCCCN(CCO)CCCCCCCCCC(=O)CC(CCCCCCCC)CCCCCCCC] |
| I24 | 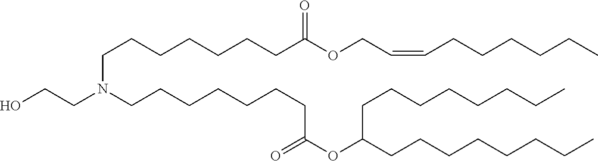 [SMILES: CCCCCC/C=C\COC(=O)CCCCCCCN(CCO)CCCCCCCC(=O)OC(CCCCCCCC)CCCCCCCC] |
| I25 | 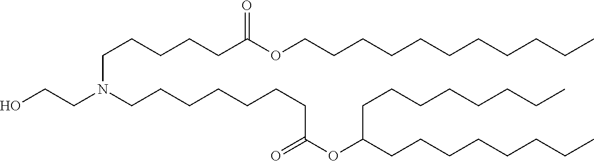 [SMILES: CCCCCCCCCCCOC(=O)CCCCCN(CCO)CCCCCCCC(=O)OC(CCCCCCCC)CCCCCCCC] |
| I26 | 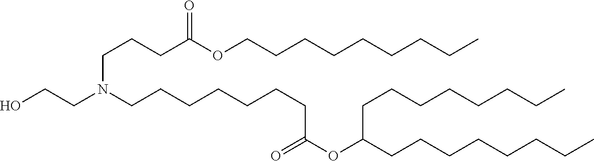 [SMILES: CCCCCCCCCOC(=O)CCCN(CCO)CCCCCCCC(=O)OC(CCCCCCCC)CCCCCCCC] |
| I27 | 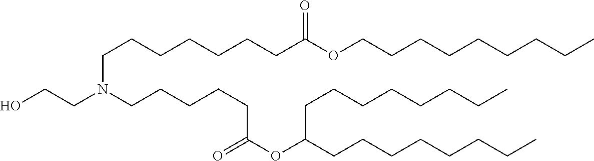 [SMILES: CCCCCCCCCOC(=O)CCCCCCCN(CCO)CCCCCC(=O)OC(CCCCCCCC)CCCCCCCC] |
| I28 | 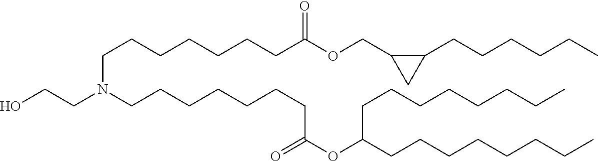 [SMILES: CCCCCCCCC(CCCCCCCC)OC(=O)CCCCCCCN(CCO)CCCCCCCC(=O)OCC1CC1CCCCCC] |
| I29 | 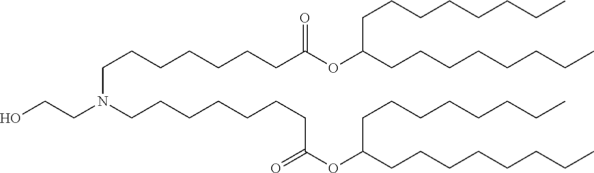 [SMILES: CCCCCCCCC(CCCCCCCC)OC(=O)CCCCCCCN(CCO)CCCCCCCC(=O)OC(CCCCCCCC)CCCCCCCC] |
| I30 | 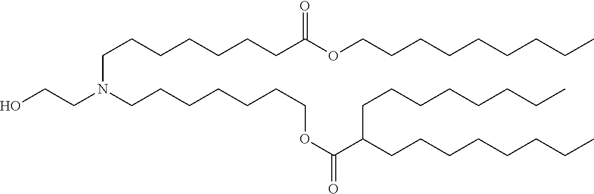 [SMILES: CCCCCCCCCOC(=O)CCCCCCCN(CCO)CCCCCCCOC(=O)C(CCCCCCCC)CCCCCCCC] |
| I31 | 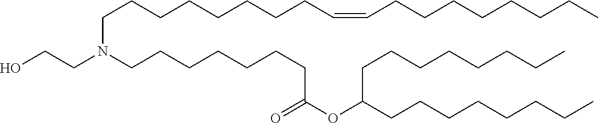 [SMILES: CCCCCCCC/C=C\CCCCCCCCN(CCO)CCCCCCCC(=O)OC(CCCCCCCC)CCCCCCCC] |
| I32 | 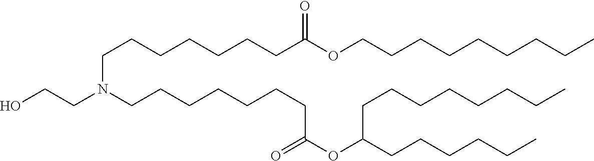 [SMILES: CCCCCCCCCOC(=O)CCCCCCCN(CCO)CCCCCCCC(=O)OC(CCCCCC)CCCCCCCC] |
| I33 | 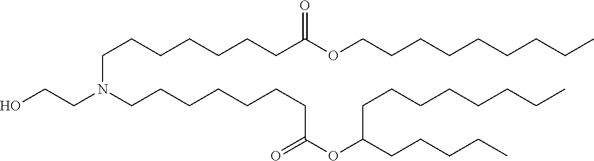 [SMILES: CCCCCCCCCOC(=O)CCCCCCCN(CCO)CCCCCCCC(=O)OC(CCCCC)CCCCCCCC] |
| I34 | 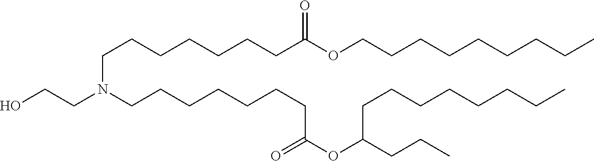 [SMILES: CCCCCCCCCOC(=O)CCCCCCCN(CCO)CCCCCCCC(=O)OC(CCC)CCCCCCCC] |
| I35 | 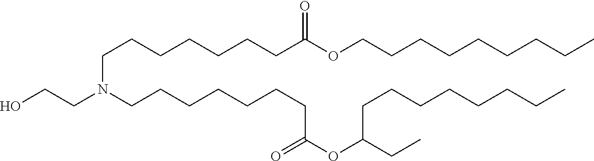 [SMILES: CCCCCCCCCOC(=O)CCCCCCCN(CCO)CCCCCCCC(=O)OC(CC)CCCCCCCC] |
| I36 | 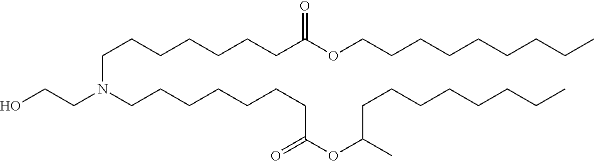 [SMILES: CCCCCCCCCOC(=O)CCCCCCCN(CCO)CCCCCCCC(=O)OC(C)CCCCCCCC] |
| I37 | 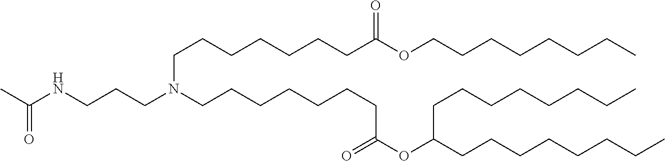 [SMILES: CCCCCCCCOC(=O)CCCCCCCN(CCCCCCCC(=O)OC(CCCCCCCC)CCCCCCCC)CCCNC(C)=O] |
| I38 | 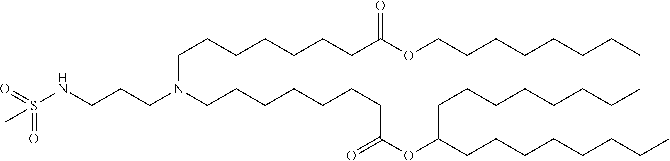 [SMILES: CCCCCCCCOC(=O)CCCCCCCN(CCCCCCCC(=O)OC(CCCCCCCC)CCCCCCCC)CCCNS(C)(=O)=O] |
| I39 | 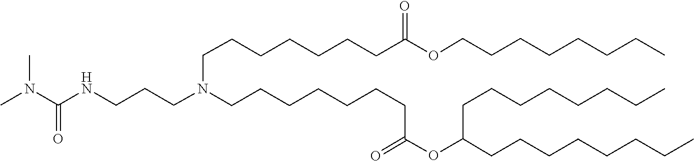 [SMILES: CCCCCCCCOC(=O)CCCCCCCN(CCCCCCCC(=O)OC(CCCCCCCC)CCCCCCCC)CCCNC(=O)N(C)C] |
| I40 | 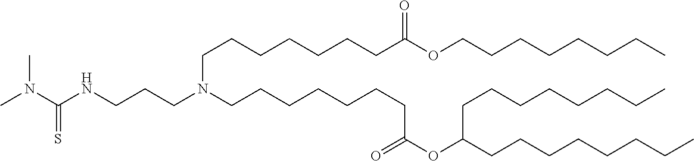 [SMILES: CCCCCCCCOC(=O)CCCCCCCN(CCCCCCCC(=O)OC(CCCCCCCC)CCCCCCCC)CCCNC(=S)N(C)C] |
| I41 | 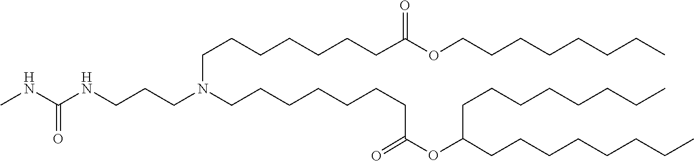 [SMILES: CCCCCCCCOC(=O)CCCCCCCN(CCCCCCCC(=O)OC(CCCCCCCC)CCCCCCCC)CCCNC(=O)NC] |
| I42 | 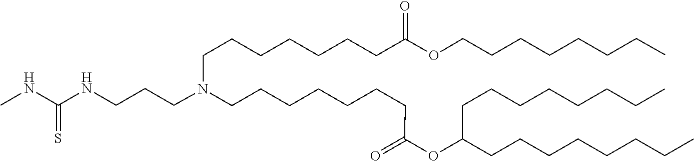 [SMILES: CCCCCCCCOC(=O)CCCCCCCN(CCCCCCCC(=O)OC(CCCCCCCC)CCCCCCCC)CCCNC(=S)NC] |
| I43 | 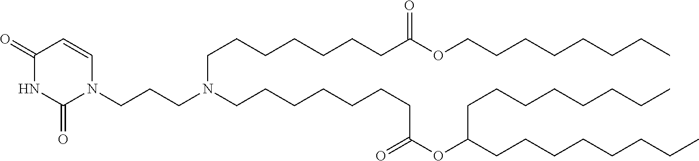 [SMILES: CCCCCCCCOC(=O)CCCCCCCN(CCCCCCCC(=O)OC(CCCCCCCC)CCCCCCCC)CCCn1ccc(=O)[nH]c1=O] |
| I44 | 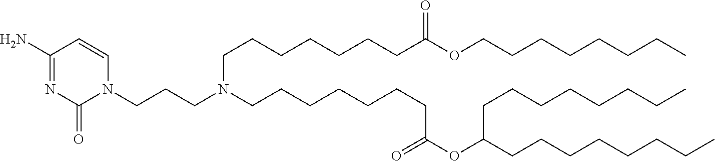 [SMILES: CCCCCCCCOC(=O)CCCCCCCN(CCCCCCCC(=O)OC(CCCCCCCC)CCCCCCCC)CCCn1ccc(N)nc1=O] |
| I45 | 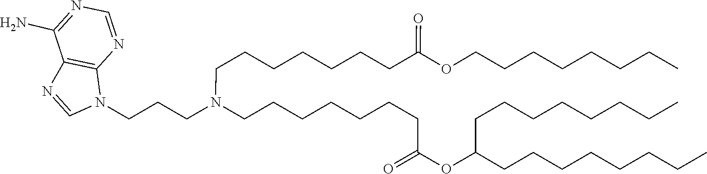 [SMILES: CCCCCCCCOC(=O)CCCCCCCN(CCCCCCCC(=O)OC(CCCCCCCC)CCCCCCCC)CCCn1cnc2c(N)ncnc21] |
| I46 | 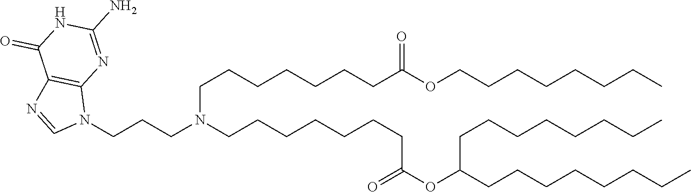 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I47 | 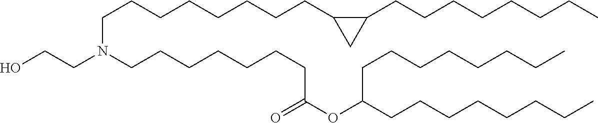 [SMILES: CCCCCCCCC(CCCCCCCC)OC(=O)CCCCCCCN(CCO)CCCCCCCCC1CC1CCCCCCCC] |
| I48 | 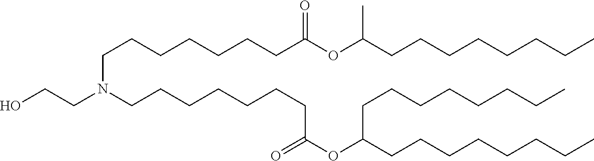 [SMILES: CCCCCCCCC(C)OC(=O)CCCCCCCN(CCO)CCCCCCCC(=O)OC(CCCCCCCC)CCCCCCCC] |
| I49 | 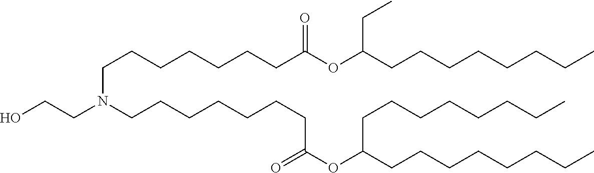 [SMILES: CCCCCCCCC(CC)OC(=O)CCCCCCCN(CCO)CCCCCCCC(=O)OC(CCCCCCCC)CCCCCCCC] |
| I50 | 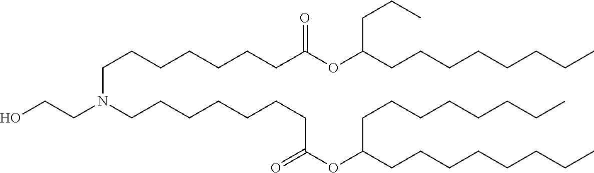 [SMILES: CCCCCCCCC(CCC)OC(=O)CCCCCCCN(CCO)CCCCCCCC(=O)OC(CCCCCCCC)CCCCCCCC] |
| I51 | 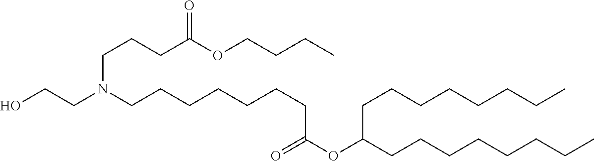 [SMILES: CCCCCCCCC(CCCCCCCC)OC(=O)CCCCCCCN(CCO)CCCC(=O)OCCCC] |
| I52 | 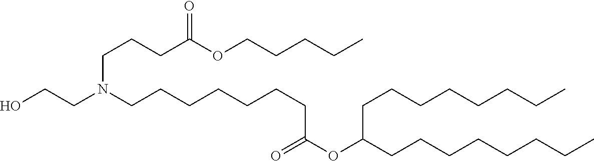 [SMILES: CCCCCCCCC(CCCCCCCC)OC(=O)CCCCCCCN(CCO)CCCC(=O)OCCCCC] |
| I53 | 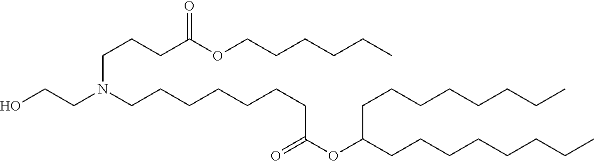 [SMILES: CCCCCCCCC(CCCCCCCC)OC(=O)CCCCCCCN(CCO)CCCC(=O)OCCCCCC] |
| I54 | 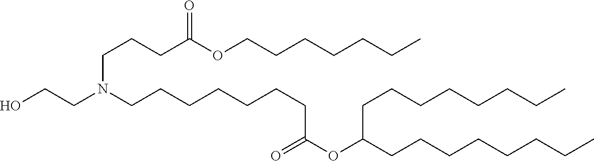 [SMILES: CCCCCCCCC(CCCCCCCC)OC(=O)CCCCCCCN(CCO)CCCC(=O)OCCCCCCC] |
| I55 | 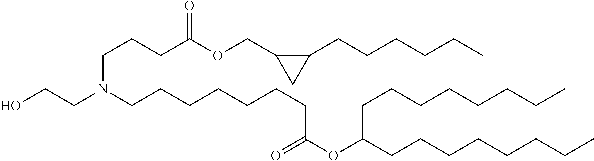 [SMILES: CCCCCCCCC(CCCCCCCC)OC(=O)CCCCCCCN(CCO)CCCC(=O)OCC1CC1CCCCCC] |
| I56 | 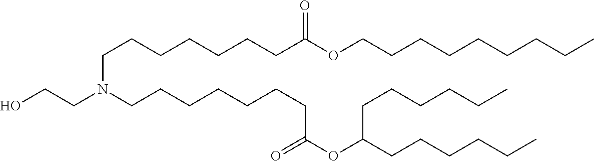 [SMILES: CCCCCCCCCOC(=O)CCCCCCCN(CCO)CCCCCCCC(=O)OC(CCCCCC)CCCCCC] |
| I57 | 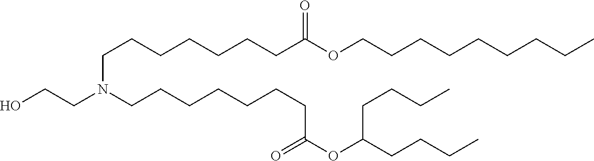 [SMILES: CCCCCCCCCOC(=O)CCCCCCCN(CCO)CCCCCCCC(=O)OC(CCCC)CCCC] |
| I58 | 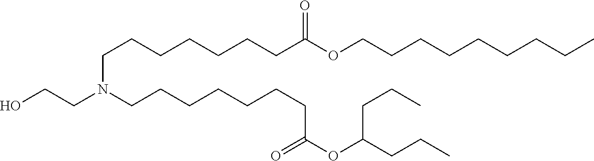 [SMILES: CCCCCCCCCOC(=O)CCCCCCCN(CCO)CCCCCCCC(=O)OC(CCC)CCC] |
| I59 | 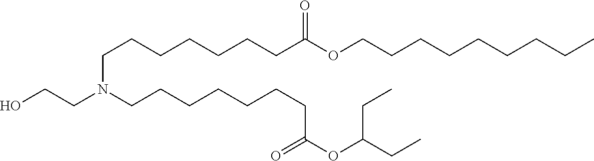 [SMILES: CCCCCCCCCOC(=O)CCCCCCCN(CCO)CCCCCCCC(=O)OC(CC)CC] |
| I60 | 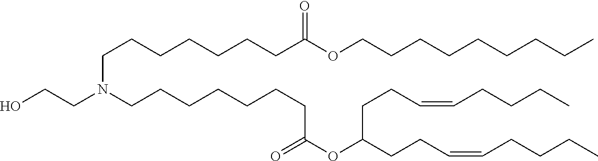 [SMILES: CCCC/C=C\CCC(CC/C=C\CCCC)OC(=O)CCCCCCCN(CCO)CCCCCCCC(=O)OCCCCCCCCC] |
| I61 | 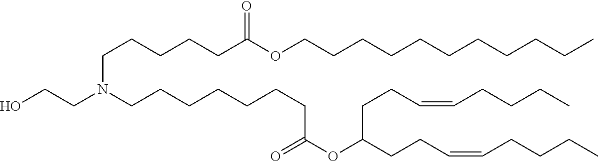 [SMILES: CCCC/C=C\CCC(CC/C=C\CCCC)OC(=O)CCCCCCCN(CCO)CCCCCC(=O)OCCCCCCCCCCC] |

In further embodiments, the compound of Formula (I I) is selected from the group consisting of: E

| Cpd | Structure |
|---|---|
| I62 | 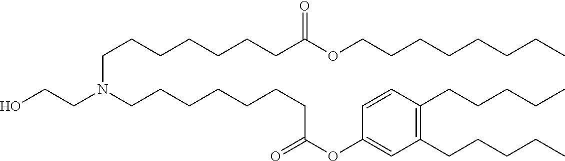 [SMILES: CCCCCCCCOC(=O)CCCCCCCN(CCO)CCCCCCCC(=O)Oc1ccc(CCCCC)c(CCCCC)c1] |
| I63 | 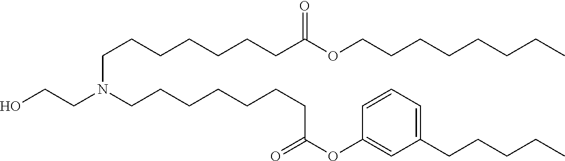 [SMILES: CCCCCCCCOC(=O)CCCCCCCN(CCO)CCCCCCCC(=O)Oc1cccc(CCCCC)c1] |
| I64 | 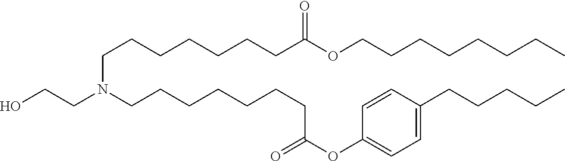 [SMILES: CCCCCCCCOC(=O)CCCCCCCN(CCO)CCCCCCCC(=O)Oc1ccc(CCCCC)cc1] |

In some embodiments, the compound of Formula (I I) or Formula (I IV) is selected from the group consisting of:

| Cpd | Structure |
|---|---|
| I65 | 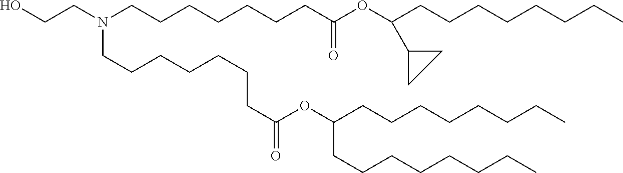 [SMILES: CCCCCCCCC(CCCCCCCC)OC(=O)CCCCCCCN(CCO)CCCCCCCC(=O)OC(CCCCCCCC)C1CC1] |
| I66 | 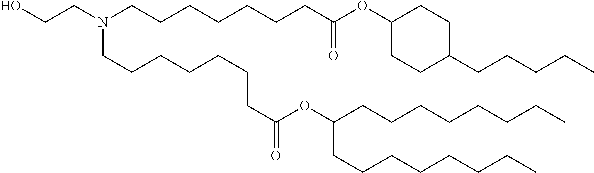 [SMILES: CCCCCCCCC(CCCCCCCC)OC(=O)CCCCCCCN(CCO)CCCCCCCC(=O)OC1CCC(CCCCC)CC1] |
| I67 | 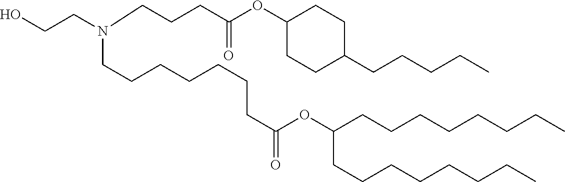 [SMILES: CCCCCCCCC(CCCCCCCC)OC(=O)CCCCCCCN(CCO)CCCC(=O)OC1CCC(CCCCC)CC1] |
| I68 | 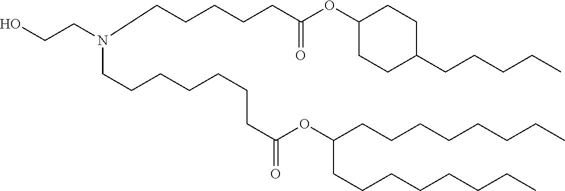 [SMILES: CCCCCCCCC(CCCCCCCC)OC(=O)CCCCCCCN(CCO)CCCCCC(=O)OC1CCC(CCCCC)CC1] |
| I69 | 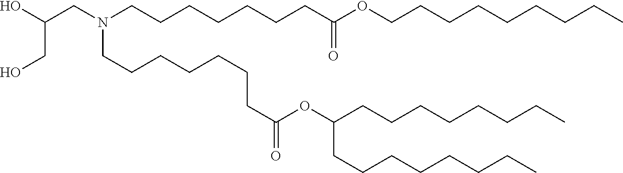 [SMILES: CCCCCCCCCOC(=O)CCCCCCCN(CCCCCCCC(=O)OC(CCCCCCCC)CCCCCCCC)CC(O)CO] |
| I70 | 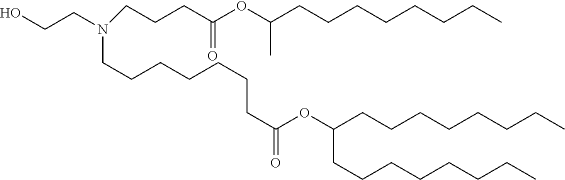 [SMILES: CCCCCCCCC(C)OC(=O)CCCN(CCO)CCCCCCCC(=O)OC(CCCCCCCC)CCCCCCCC] |
| I71 | 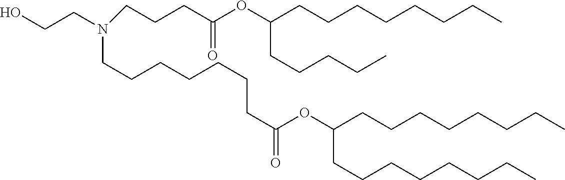 [SMILES: CCCCCCCCC(CCCCCCCC)OC(=O)CCCCCCCN(CCO)CCCC(=O)OC(CCCCC)CCCCCCCC] |
| I72 | 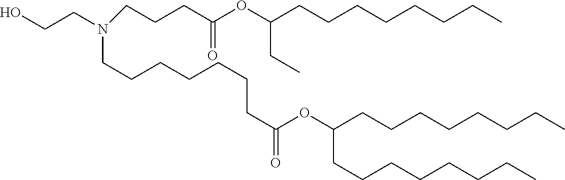 [SMILES: CCCCCCCCC(CC)OC(=O)CCCN(CCO)CCCCCCCC(=O)OC(CCCCCCCC)CCCCCCCC] |
| I73 | 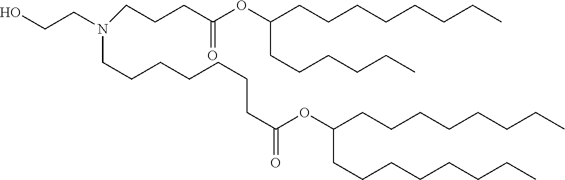 [SMILES: CCCCCCCCC(CCCCCCCC)OC(=O)CCCCCCCN(CCO)CCCC(=O)OC(CCCCCC)CCCCCCCC] |
| I74 | 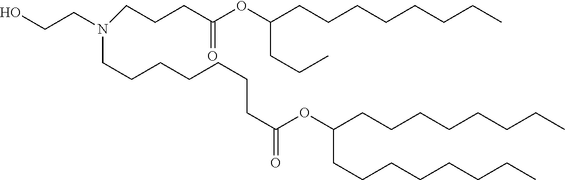 [SMILES: CCCCCCCCC(CCC)OC(=O)CCCN(CCO)CCCCCCCC(=O)OC(CCCCCCCC)CCCCCCCC] |
| I75 | 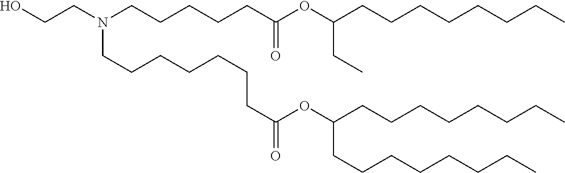 [SMILES: CCCCCCCCC(CC)OC(=O)CCCCCN(CCO)CCCCCCCC(=O)OC(CCCCCCCC)CCCCCCCC] |
| I76 | 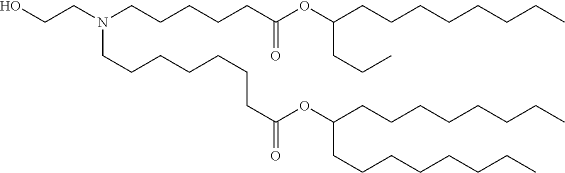 [SMILES: CCCCCCCCC(CCC)OC(=O)CCCCCN(CCO)CCCCCCCC(=O)OC(CCCCCCCC)CCCCCCCC] |
| I77 | 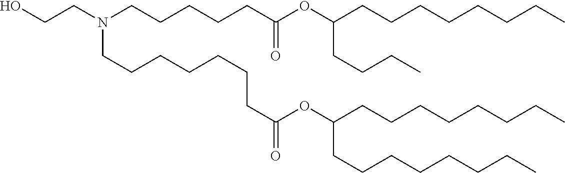 [SMILES: CCCCCCCCC(CCCC)OC(=O)CCCCCN(CCO)CCCCCCCC(=O)OC(CCCCCCCC)CCCCCCCC] |
| I78 | 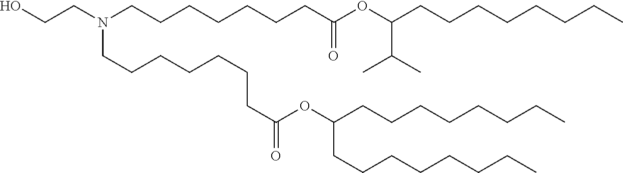 [SMILES: CCCCCCCCC(CCCCCCCC)OC(=O)CCCCCCCN(CCO)CCCCCCCC(=O)OC(CCCCCCCC)C(C)C] |
| I79 | 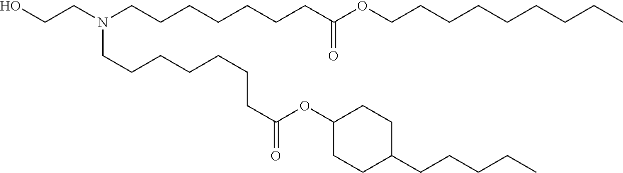 [SMILES: CCCCCCCCCOC(=O)CCCCCCCN(CCO)CCCCCCCC(=O)OC1CCC(CCCCC)CC1] |
| I80 | 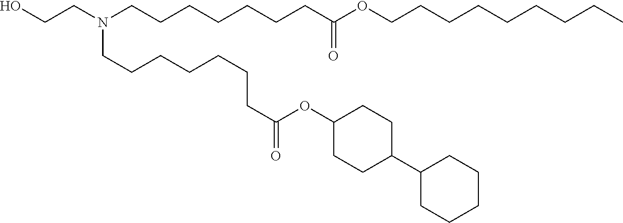 [SMILES: CCCCCCCCCOC(=O)CCCCCCCN(CCO)CCCCCCCC(=O)OC1CCC(C2CCCCC2)CC1] |
| I81 | 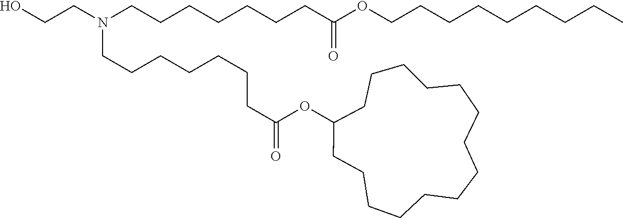 [SMILES: CCCCCCCCCOC(=O)CCCCCCCN(CCO)CCCCCCCC(=O)OC1CCCCCCCCCCCCCC1] |
| I82 | 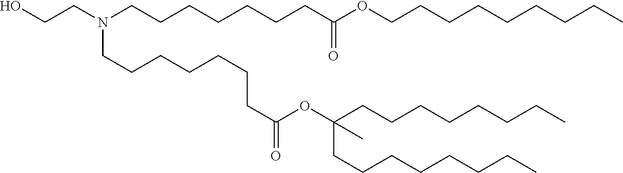 [SMILES: CCCCCCCCCOC(=O)CCCCCCCN(CCO)CCCCCCCC(=O)OC(C)(CCCCCCCC)CCCCCCCC] |
| I83 | 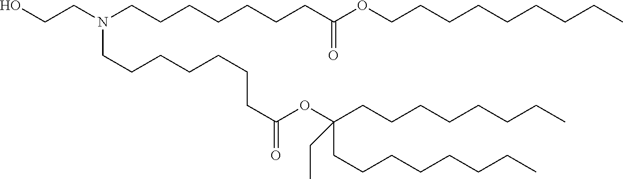 [SMILES: CCCCCCCCCOC(=O)CCCCCCCN(CCO)CCCCCCCC(=O)OC(CC)(CCCCCCCC)CCCCCCCC] |
| I84 | 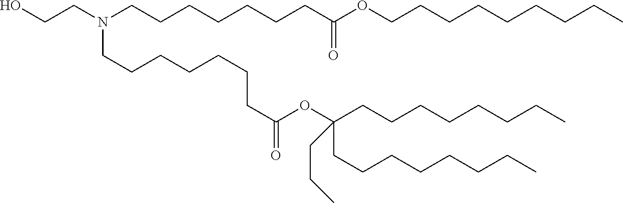 [SMILES: CCCCCCCCCOC(=O)CCCCCCCN(CCO)CCCCCCCC(=O)OC(CCC)(CCCCCCCC)CCCCCCCC] |
| I85 | 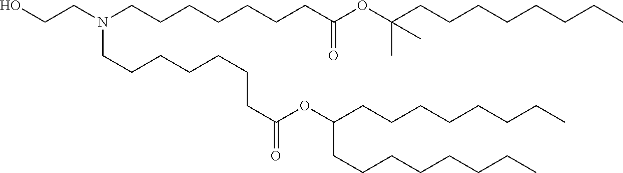 [SMILES: CCCCCCCCC(CCCCCCCC)OC(=O)CCCCCCCN(CCO)CCCCCCCC(=O)OC(C)(C)CCCCCCCC] |
| I86 | 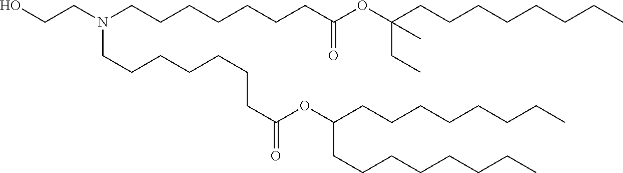 [SMILES: CCCCCCCCC(CCCCCCCC)OC(=O)CCCCCCCN(CCO)CCCCCCCC(=O)OC(C)(CC)CCCCCCCC] |
| I87 | 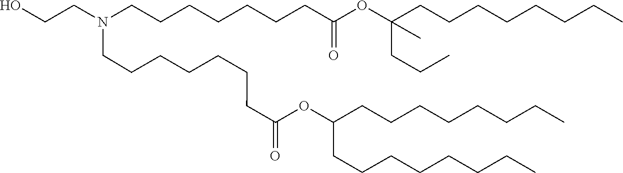 [SMILES: CCCCCCCCC(CCCCCCCC)OC(=O)CCCCCCCN(CCO)CCCCCCCC(=O)OC(C)(CCC)CCCCCCCC] |
| I88 | 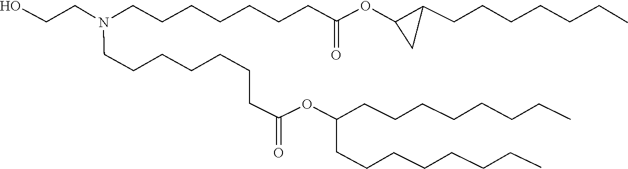 [SMILES: CCCCCCCCC(CCCCCCCC)OC(=O)CCCCCCCN(CCO)CCCCCCCC(=O)OC1CC1CCCCCCC] |
| I89 | 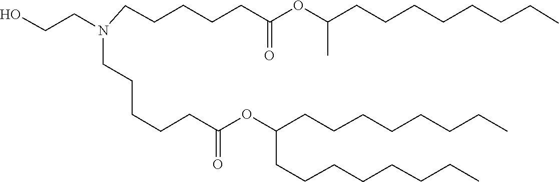 [SMILES: CCCCCCCCC(C)OC(=O)CCCCCN(CCO)CCCCCC(=O)OC(CCCCCCCC)CCCCCCCC] |
| I90 | 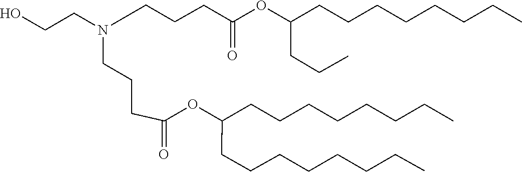 [SMILES: CCCCCCCCC(CCC)OC(=O)CCCN(CCO)CCCC(=O)OC(CCCCCCCC)CCCCCCCC] |
| I91 | 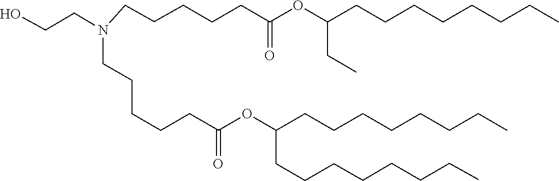 [SMILES: CCCCCCCCC(CC)OC(=O)CCCCCN(CCO)CCCCCC(=O)OC(CCCCCCCC)CCCCCCCC] |
| I92 | 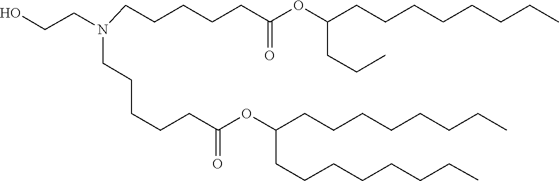 [SMILES: CCCCCCCCC(CCC)OC(=O)CCCCCN(CCO)CCCCCC(=O)OC(CCCCCCCC)CCCCCCCC] |
| I93 | 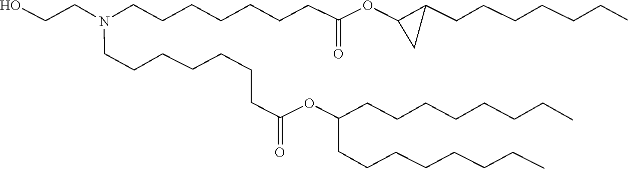 [SMILES: CCCCCCCCC(CCCCCCCC)OC(=O)CCCCCCCN(CCO)CCCCCCCC(=O)OC1CC1CCCCCCC] |
| I94 | 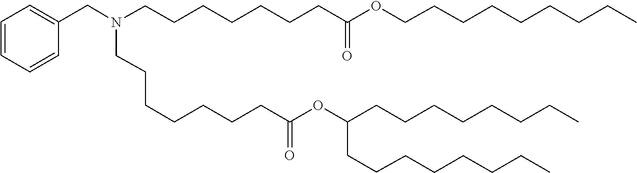 [SMILES: CCCCCCCCCOC(=O)CCCCCCCN(CCCCCCCC(=O)OC(CCCCCCCC)CCCCCCCC)Cc1ccccc1] |
| I95 | 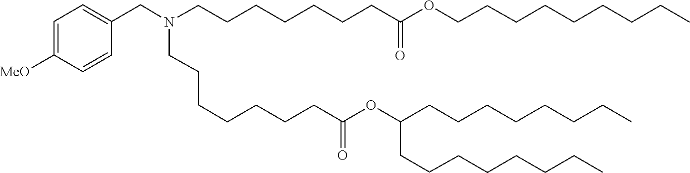 [SMILES: CCCCCCCCCOC(=O)CCCCCCCN(CCCCCCCC(=O)OC(CCCCCCCC)CCCCCCCC)Cc1ccc(OC)cc1] |
| I96 | 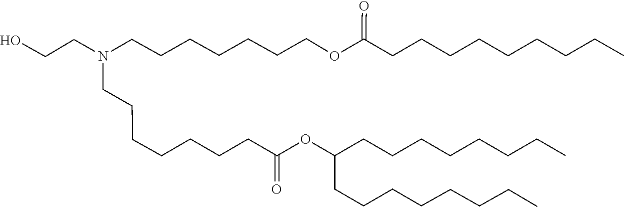 [SMILES: CCCCCCCCCC(=O)OCCCCCCCN(CCO)CCCCCCCC(=O)OC(CCCCCCCC)CCCCCCCC] |
| I97 | 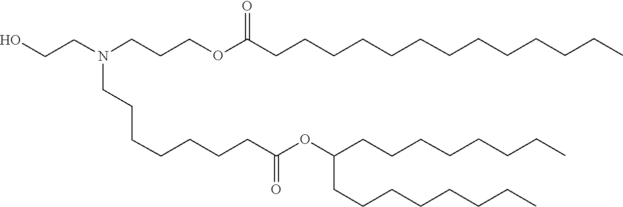 [SMILES: CCCCCCCCCCCCCC(=O)OCCCN(CCO)CCCCCCCC(=O)OC(CCCCCCCC)CCCCCCCC] |
| I98 | 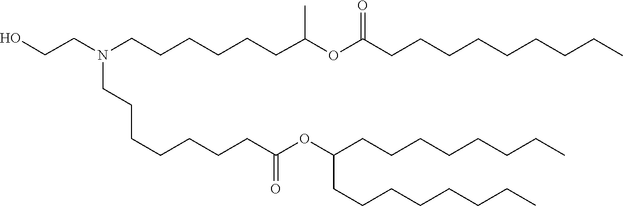 [SMILES: CCCCCCCCCC(=O)OC(C)CCCCCCN(CCO)CCCCCCCC(=O)OC(CCCCCCCC)CCCCCCCC] |
| I99 | 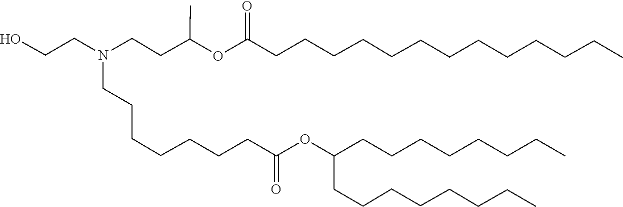 [SMILES: CCCCCCCCCCCCCC(=O)OC(C)CCN(CCO)CCCCCCCC(=O)OC(CCCCCCCC)CCCCCCCC] |
| I100 | 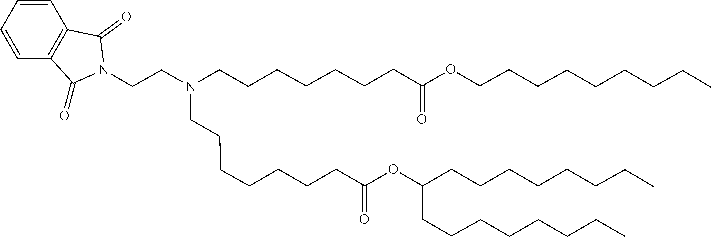 [SMILES: CCCCCCCCCOC(=O)CCCCCCCN(CCCCCCCC(=O)OC(CCCCCCCC)CCCCCCCC)CCN1C(=O)c2ccccc2C1=O] |
| I101 | 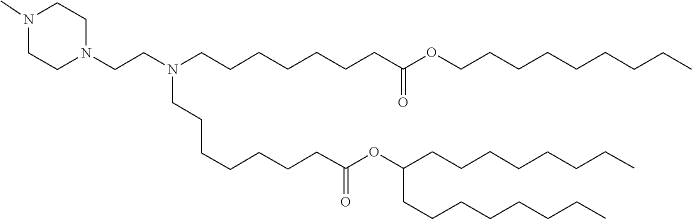 [SMILES: CCCCCCCCCOC(=O)CCCCCCCN(CCCCCCCC(=O)OC(CCCCCCCC)CCCCCCCC)CCN1CCN(C)CC1] |
| I102 | 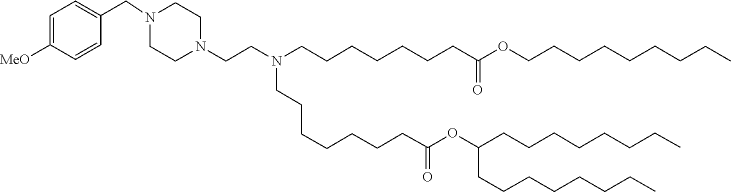 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I103 | 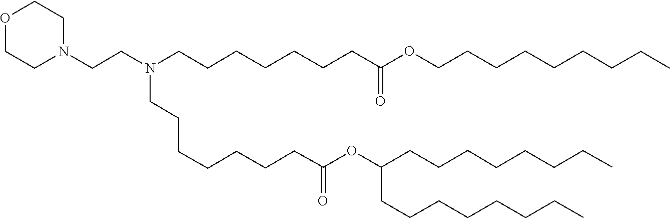 [SMILES: CCCCCCCCCOC(=O)CCCCCCCN(CCCCCCCC(=O)OC(CCCCCCCC)CCCCCCCC)CCN1CCOCC1] |
| I104 | 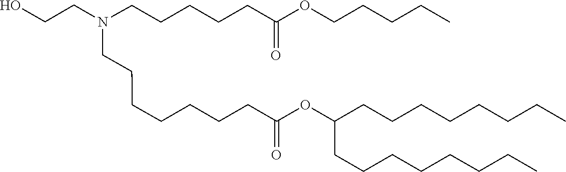 [SMILES: CCCCCCCCC(CCCCCCCC)OC(=O)CCCCCCCN(CCO)CCCCCC(=O)OCCCCC] |
| I105 | 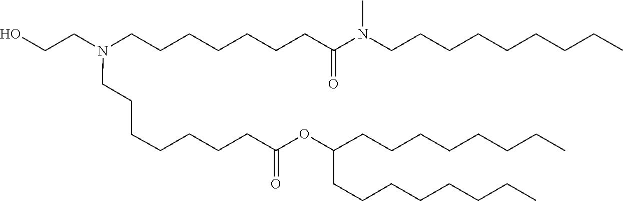 [SMILES: CCCCCCCCCN(C)C(=O)CCCCCCCN(CCO)CCCCCCCC(=O)OC(CCCCCCCC)CCCCCCCC] |
| I106 | 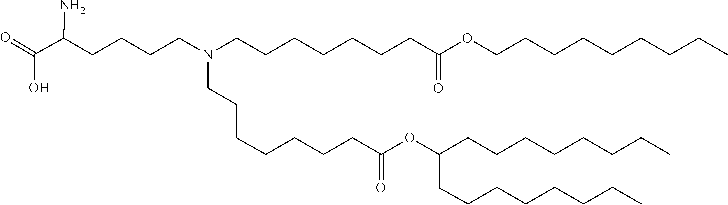 [SMILES: CCCCCCCCCOC(=O)CCCCCCCN(CCCCCCCC(=O)OC(CCCCCCCC)CCCCCCCC)CCCCC(N)C(=O)O] |
| I107 | 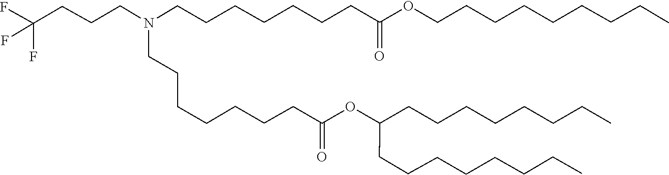 [SMILES: CCCCCCCCCOC(=O)CCCCCCCN(CCCCCCCC(=O)OC(CCCCCCCC)CCCCCCCC)CCCC(F)(F)F] |
| I108 | 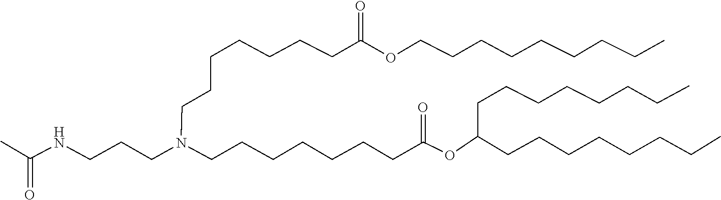 [SMILES: CCCCCCCCCOC(=O)CCCCCCCN(CCCCCCCC(=O)OC(CCCCCCCC)CCCCCCCC)CCCNC(C)=O] |
| I109 | 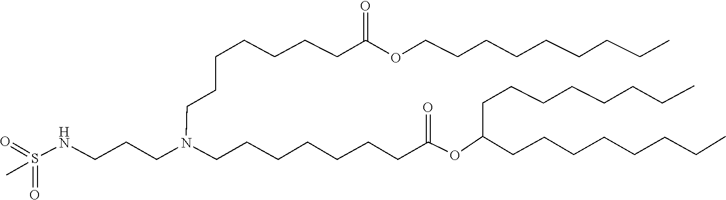 [SMILES: CCCCCCCCCOC(=O)CCCCCCCN(CCCCCCCC(=O)OC(CCCCCCCC)CCCCCCCC)CCCNS(C)(=O)=O] |
| I110 | 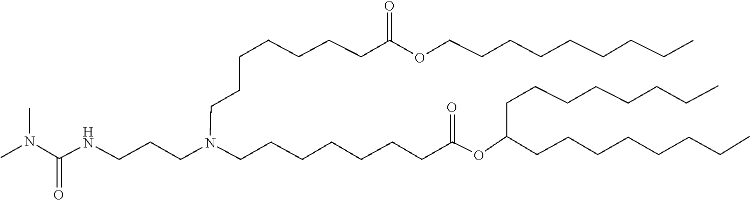 [SMILES: CCCCCCCCCOC(=O)CCCCCCCN(CCCCCCCC(=O)OC(CCCCCCCC)CCCCCCCC)CCCNC(=O)N(C)C] |
| I111 | 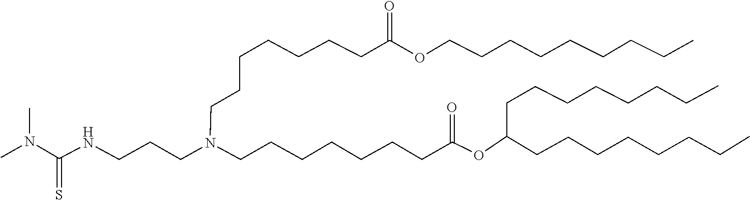 [SMILES: CCCCCCCCCOC(=O)CCCCCCCN(CCCCCCCC(=O)OC(CCCCCCCC)CCCCCCCC)CCCNC(=S)N(C)C] |
| I112 | 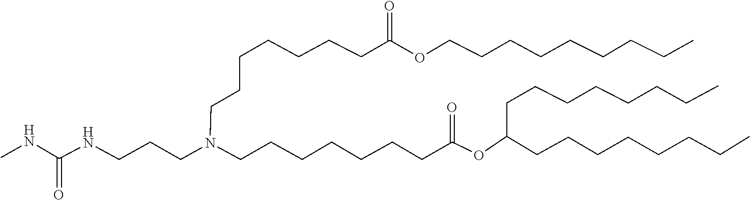 [SMILES: CCCCCCCCCOC(=O)CCCCCCCN(CCCCCCCC(=O)OC(CCCCCCCC)CCCCCCCC)CCCNC(=O)NC] |
| I113 | 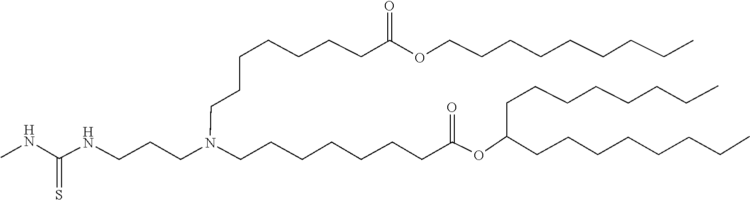 [SMILES: CCCCCCCCCOC(=O)CCCCCCCN(CCCCCCCC(=O)OC(CCCCCCCC)CCCCCCCC)CCCNC(=S)NC] |
| I114 | 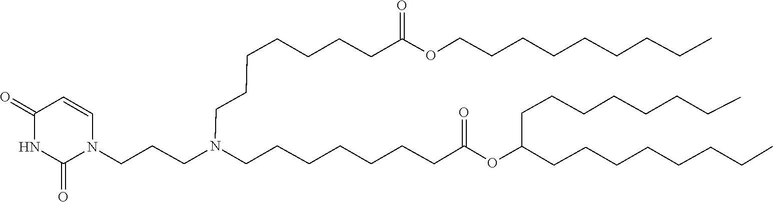 [SMILES: CCCCCCCCCOC(=O)CCCCCCCN(CCCCCCCC(=O)OC(CCCCCCCC)CCCCCCCC)CCCn1ccc(=O)[nH]c1=O] |
| I115 | 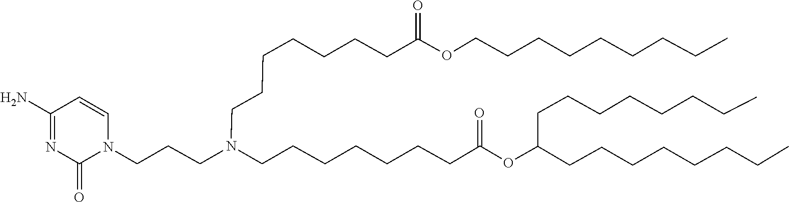 [SMILES: CCCCCCCCCOC(=O)CCCCCCCN(CCCCCCCC(=O)OC(CCCCCCCC)CCCCCCCC)CCCn1ccc(N)nc1=O] |
| I116 | 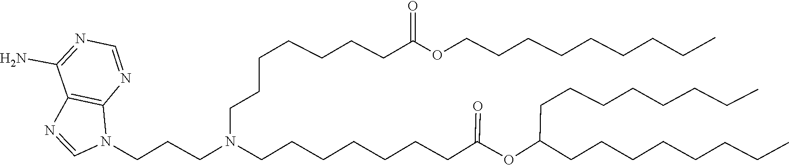 [SMILES: CCCCCCCCCOC(=O)CCCCCCCN(CCCCCCCC(=O)OC(CCCCCCCC)CCCCCCCC)CCCn1cnc2c(N)ncnc21] |
| I117 | 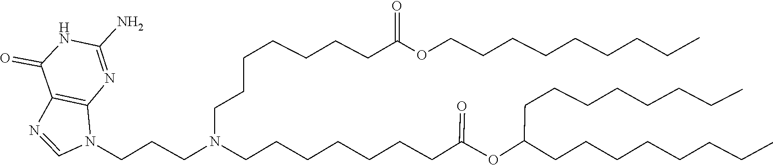 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I118 | 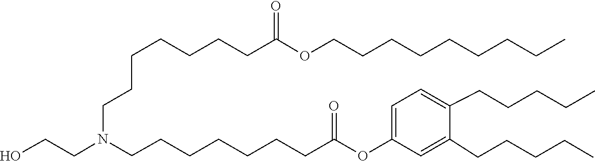 [SMILES: CCCCCCCCCOC(=O)CCCCCCCN(CCO)CCCCCCCC(=O)Oc1ccc(CCCCC)c(CCCCC)c1] |
| I119 | 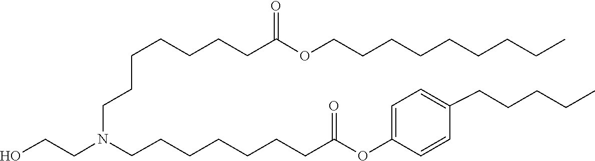 [SMILES: CCCCCCCCCOC(=O)CCCCCCCN(CCO)CCCCCCCC(=O)Oc1ccc(CCCCC)cc1] |
| I120 | 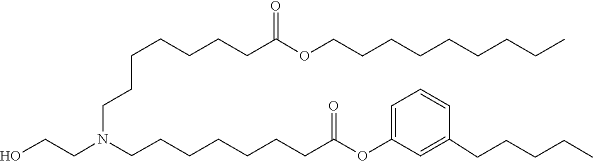 [SMILES: CCCCCCCCCOC(=O)CCCCCCCN(CCO)CCCCCCCC(=O)Oc1cccc(CCCCC)c1] |
| I121 | 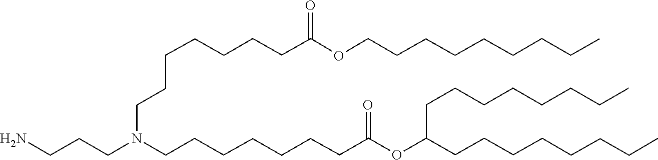 [SMILES: CCCCCCCCCOC(=O)CCCCCCCN(CCCN)CCCCCCCC(=O)OC(CCCCCCCC)CCCCCCCC] |
| I122 | 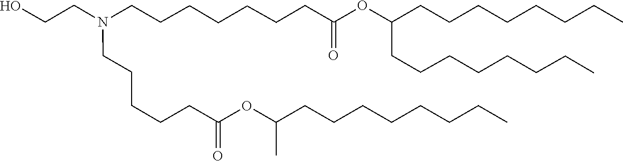 [SMILES: CCCCCCCCC(C)OC(=O)CCCCCN(CCO)CCCCCCCC(=O)OC(CCCCCCCC)CCCCCCCC] |
| I123 | 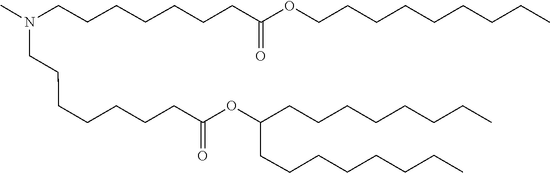 [SMILES: CCCCCCCCCOC(=O)CCCCCCCN(C)CCCCCCCC(=O)OC(CCCCCCCC)CCCCCCCC] |
| I124 | 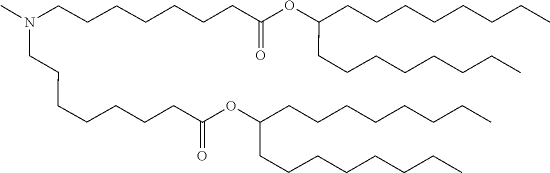 [SMILES: CCCCCCCCC(CCCCCCCC)OC(=O)CCCCCCCN(C)CCCCCCCC(=O)OC(CCCCCCCC)CCCCCCCC] |
| I125 | 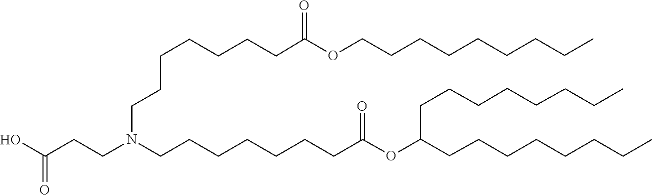 [SMILES: CCCCCCCCCOC(=O)CCCCCCCN(CCCCCCCC(=O)OC(CCCCCCCC)CCCCCCCC)CCC(=O)O] |
| I126 | 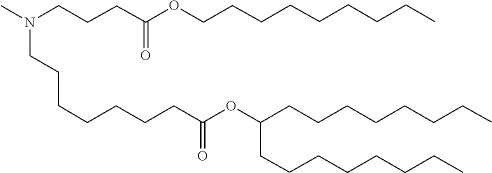 [SMILES: CCCCCCCCCOC(=O)CCCN(C)CCCCCCCC(=O)OC(CCCCCCCC)CCCCCCCC] |
| I127 | 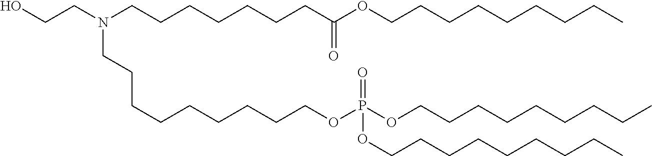 [SMILES: CCCCCCCCCOC(=O)CCCCCCCN(CCO)CCCCCCCCCOP(=O)(OCCCCCCCCC)OCCCCCCCCC] |
| I128 | 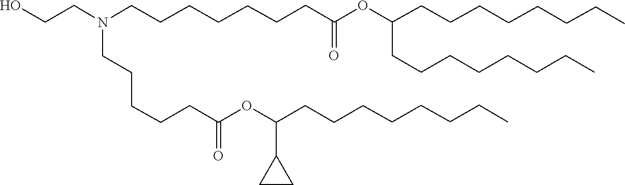 [SMILES: CCCCCCCCC(CCCCCCCC)OC(=O)CCCCCCCN(CCO)CCCCCC(=O)OC(CCCCCCCC)C1CC1] |
| I129 | 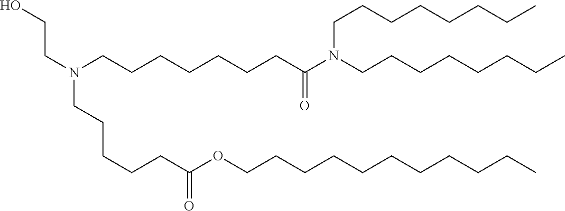 [SMILES: CCCCCCCCCCCOC(=O)CCCCCN(CCO)CCCCCCCC(=O)N(CCCCCCCC)CCCCCCCC] |
| I130 | 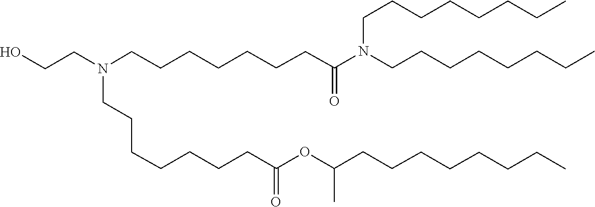 [SMILES: CCCCCCCCC(C)OC(=O)CCCCCCCN(CCO)CCCCCCCC(=O)N(CCCCCCCC)CCCCCCCC] |
| I131 | 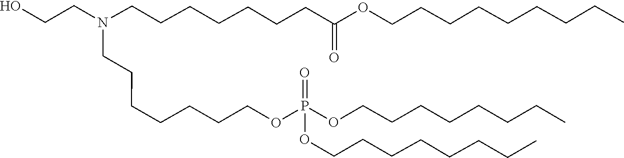 [SMILES: CCCCCCCCCOC(=O)CCCCCCCN(CCO)CCCCCCCOP(=O)(OCCCCCCCC)OCCCCCCCC] |
| I132 | 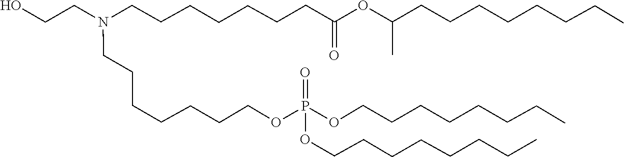 [SMILES: CCCCCCCCOP(=O)(OCCCCCCCC)OCCCCCCCN(CCO)CCCCCCCC(=O)OC(C)CCCCCCCC] |
| I133 | 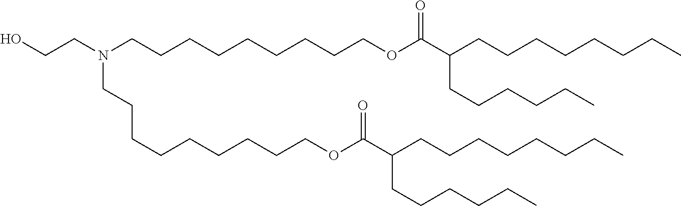 [SMILES: CCCCCCCCC(CCCCCC)C(=O)OCCCCCCCCCN(CCO)CCCCCCCCCOC(=O)C(CCCCCC)CCCCCCCC] |
| I134 | 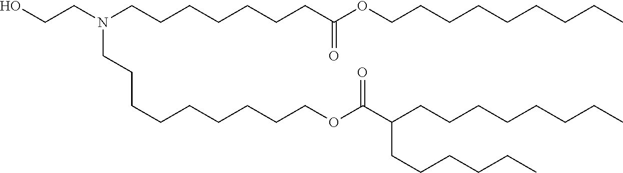 [SMILES: CCCCCCCCCOC(=O)CCCCCCCN(CCO)CCCCCCCCCOC(=O)C(CCCCCC)CCCCCCCC] |
| I135 | 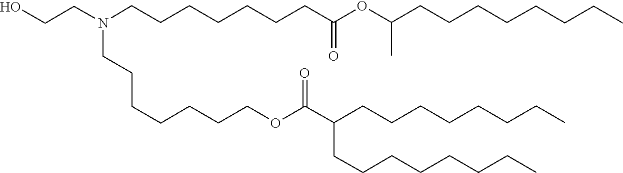 [SMILES: CCCCCCCCC(C)OC(=O)CCCCCCCN(CCO)CCCCCCCOC(=O)C(CCCCCCCC)CCCCCCCC] |
| I136 | 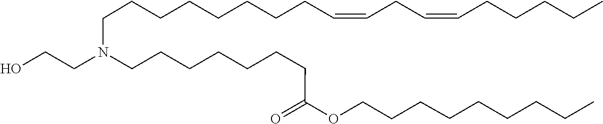 [SMILES: CCCCC/C=C\C/C=C\CCCCCCCCN(CCO)CCCCCCCC(=O)OCCCCCCCCC] |
| I137 | 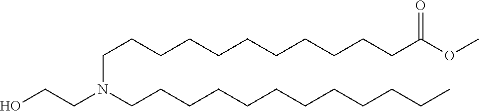 [SMILES: CCCCCCCCCCCCN(CCO)CCCCCCCCCCCC(=O)OC] |
| I138 | 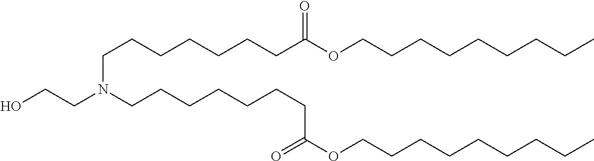 [SMILES: CCCCCCCCCOC(=O)CCCCCCCN(CCO)CCCCCCCC(=O)OCCCCCCCCC] |
| I139 | 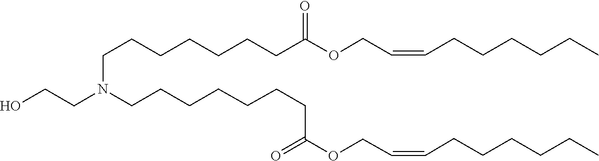 [SMILES: CCCCCC/C=C\COC(=O)CCCCCCCN(CCO)CCCCCCCC(=O)OC/C=C\CCCCCC] |
| I140 | 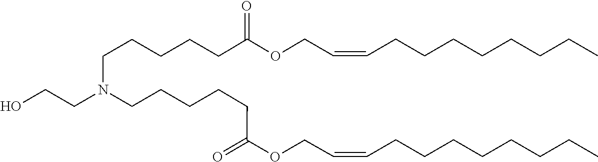 [SMILES: CCCCCCCC/C=C\COC(=O)CCCCCN(CCO)CCCCCC(=O)OC/C=C\CCCCCCCC] |
| I141 | 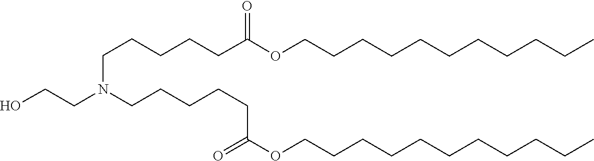 [SMILES: CCCCCCCCCCCOC(=O)CCCCCN(CCO)CCCCCC(=O)OCCCCCCCCCCC] |
| I142 | 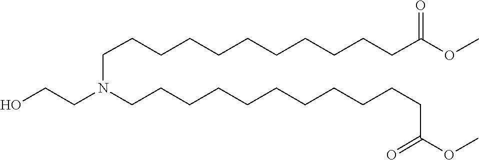 [SMILES: COC(=O)CCCCCCCCCCCN(CCO)CCCCCCCCCCCC(=O)OC] |
| I143 | 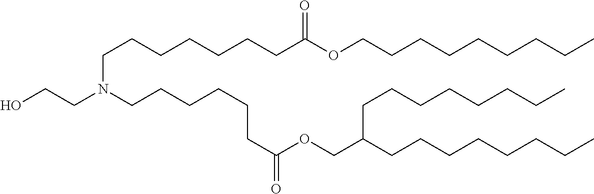 [SMILES: CCCCCCCCCOC(=O)CCCCCCCN(CCO)CCCCCCC(=O)OCC(CCCCCCCC)CCCCCCCC] |
| I144 | 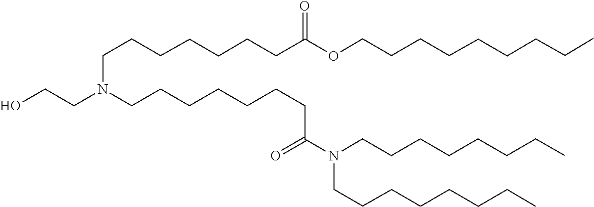 [SMILES: CCCCCCCCCOC(=O)CCCCCCCN(CCO)CCCCCCCC(=O)N(CCCCCCCC)CCCCCCCC] |
| I145 | 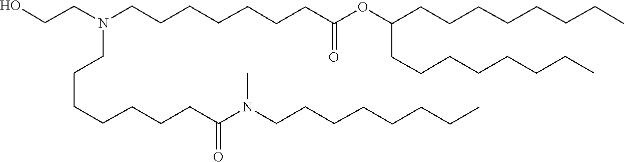 [SMILES: CCCCCCCCC(CCCCCCCC)OC(=O)CCCCCCCN(CCO)CCCCCCCC(=O)N(C)CCCCCCCC] |
| I146 | 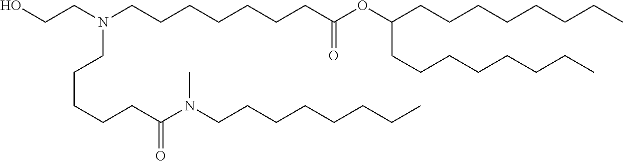 [SMILES: CCCCCCCCC(CCCCCCCC)OC(=O)CCCCCCCN(CCO)CCCCCC(=O)N(C)CCCCCCCC] |
| I147 | 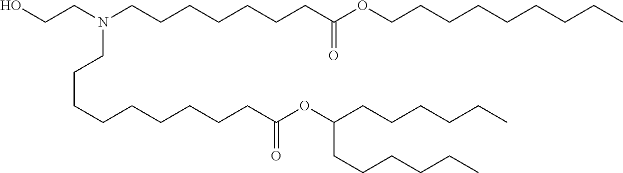 [SMILES: CCCCCCCCCOC(=O)CCCCCCCN(CCO)CCCCCCCCCC(=O)OC(CCCCCC)CCCCCC] |
| I148 | 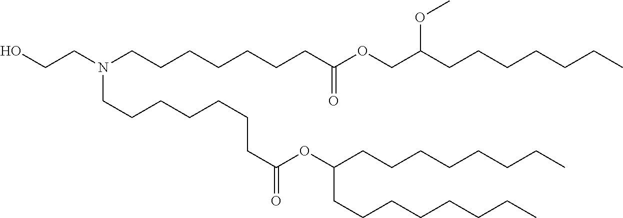 [SMILES: CCCCCCCCC(CCCCCCCC)OC(=O)CCCCCCCN(CCO)CCCCCCCC(=O)OCC(CCCCCCC)OC] |
| I149 | 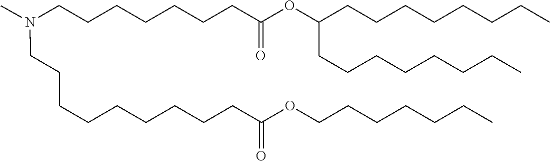 [SMILES: CCCCCCCCC(CCCCCCCC)OC(=O)CCCCCCCN(C)CCCCCCCCCC(=O)OCCCCCCC] |
| I150 | 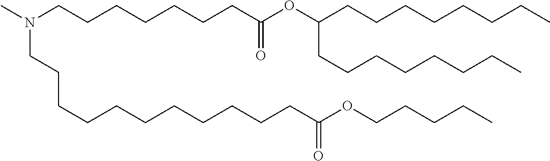 [SMILES: CCCCCCCCC(CCCCCCCC)OC(=O)CCCCCCCN(C)CCCCCCCCCCCC(=O)OCCCCC] |
| I151 | 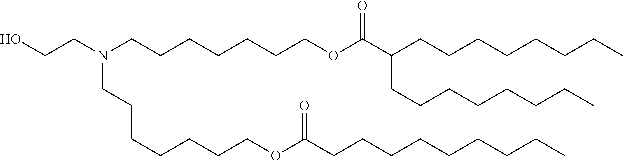 [SMILES: CCCCCCCCCC(=O)OCCCCCCCN(CCO)CCCCCCCOC(=O)C(CCCCCCCC)CCCCCCCC] |
| I152 | 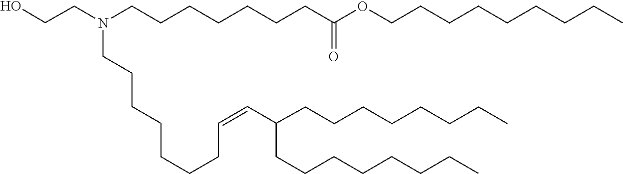 [SMILES: CCCCCCCCCOC(=O)CCCCCCCN(CCO)CCCCCCC/C=C\C(CCCCCCCC)CCCCCCCC] |
| I153 | 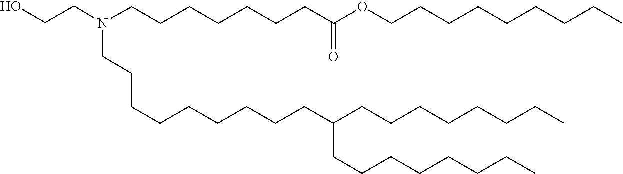 [SMILES: CCCCCCCCCOC(=O)CCCCCCCN(CCO)CCCCCCCCCC(CCCCCCCC)CCCCCCCC] |
| I154 | 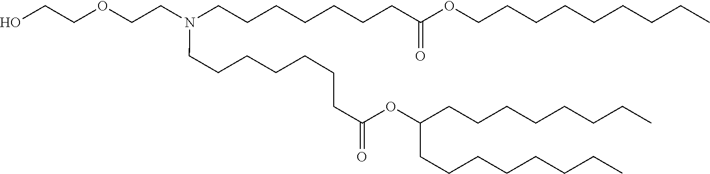 [SMILES: CCCCCCCCCOC(=O)CCCCCCCN(CCCCCCCC(=O)OC(CCCCCCCC)CCCCCCCC)CCOCCO] |
| I155 | 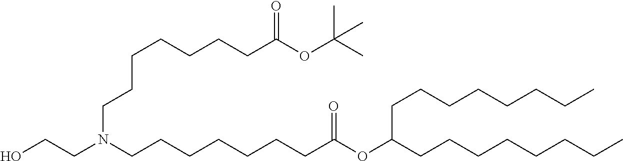 [SMILES: CCCCCCCCC(CCCCCCCC)OC(=O)CCCCCCCN(CCO)CCCCCCCC(=O)OC(C)(C)C] |
| I156 | 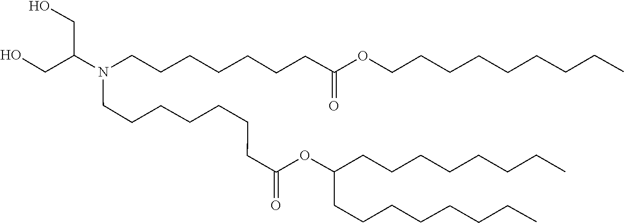 [SMILES: CCCCCCCCCOC(=O)CCCCCCCN(CCCCCCCC(=O)OC(CCCCCCCC)CCCCCCCC)C(CO)CO] |
| I157 | 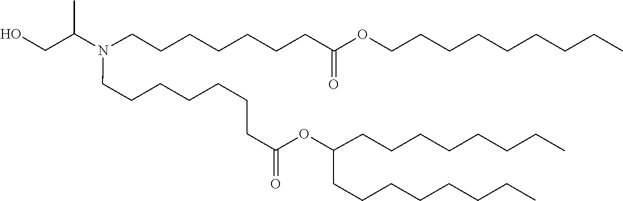 [SMILES: CCCCCCCCCOC(=O)CCCCCCCN(CCCCCCCC(=O)OC(CCCCCCCC)CCCCCCCC)C(C)CO] |
| I158 | 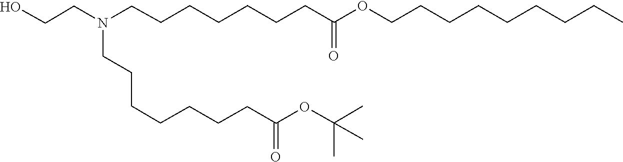 [SMILES: CCCCCCCCCOC(=O)CCCCCCCN(CCO)CCCCCCCC(=O)OC(C)(C)C] |
| I159 | 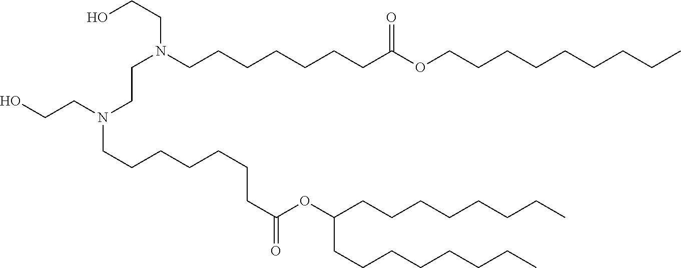 [SMILES: CCCCCCCCCOC(=O)CCCCCCCN(CCO)CCN(CCO)CCCCCCCC(=O)OC(CCCCCCCC)CCCCCCCC] |
| I160 | 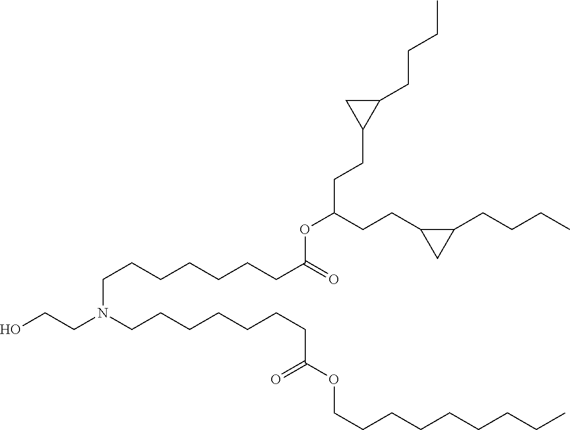 [SMILES: CCCCCCCCCOC(=O)CCCCCCCN(CCO)CCCCCCCC(=O)OC(CCC1CC1CCCC)CCC1CC1CCCC] |
| I161 | 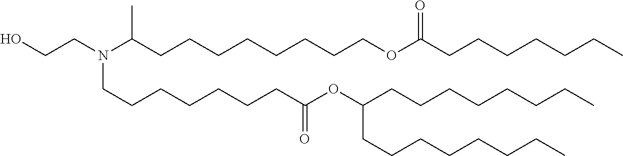 [SMILES: CCCCCCCCC(CCCCCCCC)OC(=O)CCCCCCCN(CCO)C(C)CCCCCCCCOC(=O)CCCCCCC] |
| I162 | 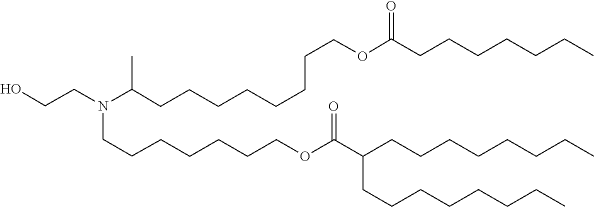 [SMILES: CCCCCCCCC(CCCCCCCC)C(=O)OCCCCCCCN(CCO)C(C)CCCCCCCCOC(=O)CCCCCCC] |
| I163 | 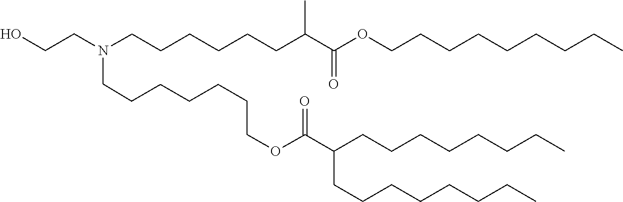 [SMILES: CCCCCCCCCOC(=O)C(C)CCCCCCN(CCO)CCCCCCCOC(=O)C(CCCCCCCC)CCCCCCCC] |
| I164 | 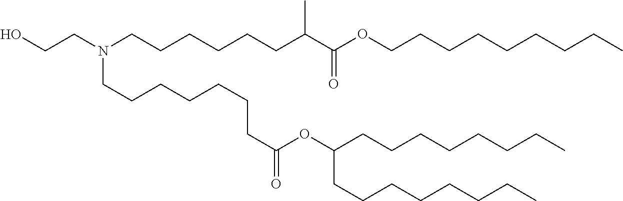 [SMILES: CCCCCCCCCOC(=O)C(C)CCCCCCN(CCO)CCCCCCCC(=O)OC(CCCCCCCC)CCCCCCCC] |
| I165 | 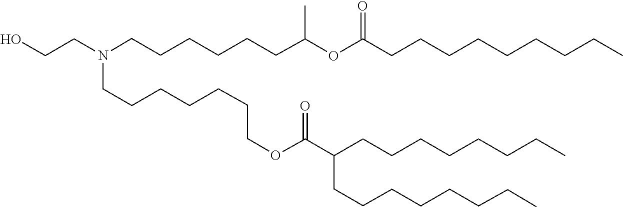 [SMILES: CCCCCCCCCC(=O)OC(C)CCCCCCN(CCO)CCCCCCCOC(=O)C(CCCCCCCC)CCCCCCCC] |
| I166 | 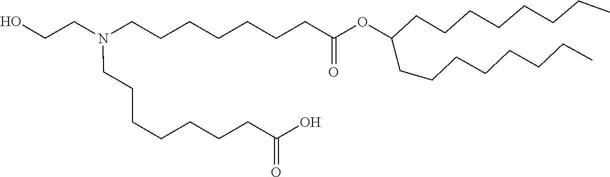 [SMILES: CCCCCCCCC(CCCCCCCC)OC(=O)CCCCCCCN(CCO)CCCCCCCC(=O)O] |
| I167 | 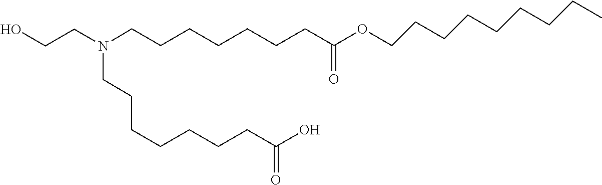 [SMILES: CCCCCCCCCOC(=O)CCCCCCCN(CCO)CCCCCCCC(=O)O] |
| I168 | 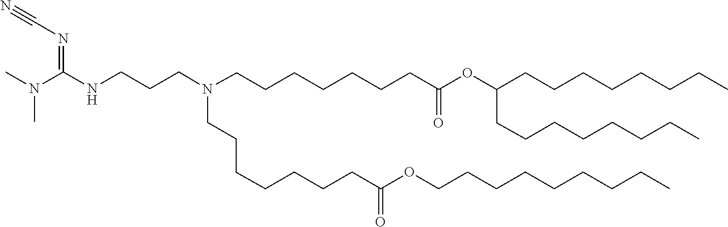 [SMILES: CCCCCCCCCOC(=O)CCCCCCCN(CCCCCCCC(=O)OC(CCCCCCCC)CCCCCCCC)CCCN/C(=N/C#N)N(C)C] |
| I169 | 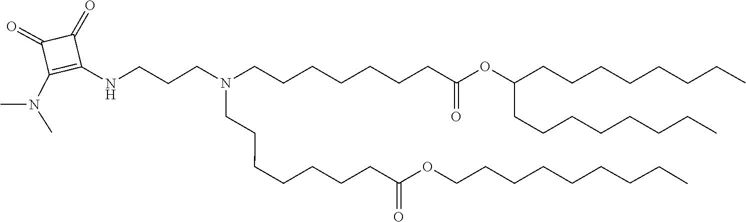 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I170 | 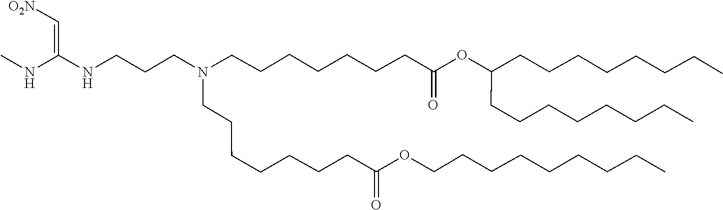 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I171 | 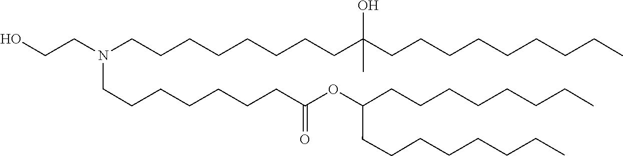 [SMILES: CCCCCCCCCC(C)(O)CCCCCCCCN(CCO)CCCCCCCC(=O)OC(CCCCCCCC)CCCCCCCC] |
| I172 | 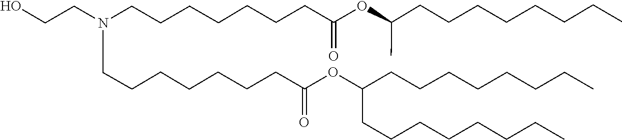 [SMILES: CCCCCCCCC(CCCCCCCC)OC(=O)CCCCCCCN(CCO)CCCCCCCC(=O)O[C@H](C)CCCCCCCC] |
| I173 | 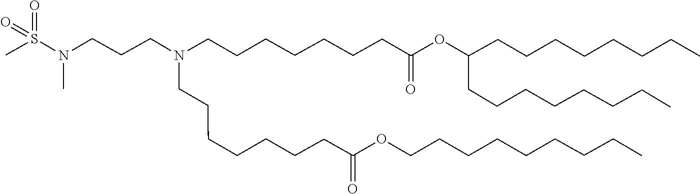 [SMILES: CCCCCCCCCOC(=O)CCCCCCCN(CCCCCCCC(=O)OC(CCCCCCCC)CCCCCCCC)CCCN(C)S(C)(=O)=O] |
| I174 | 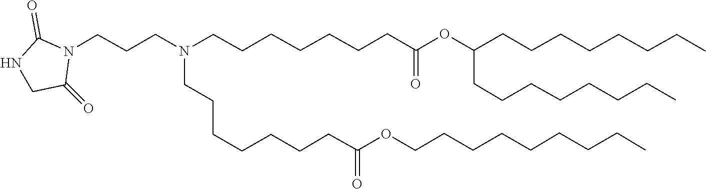 [SMILES: CCCCCCCCCOC(=O)CCCCCCCN(CCCCCCCC(=O)OC(CCCCCCCC)CCCCCCCC)CCCN1C(=O)CNC1=O] |
| I175 | 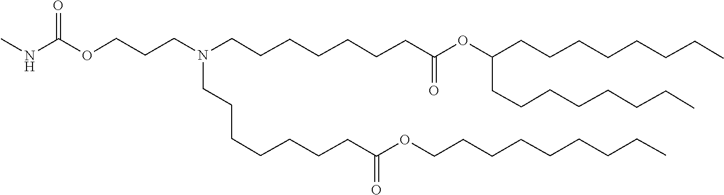 [SMILES: CCCCCCCCCOC(=O)CCCCCCCN(CCCCCCCC(=O)OC(CCCCCCCC)CCCCCCCC)CCCOC(=O)NC] |
| I176 | 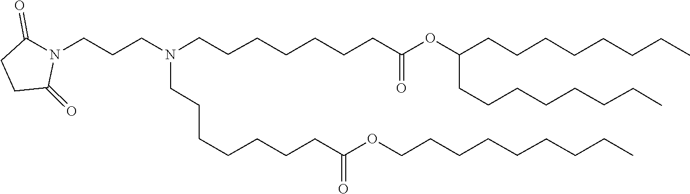 [SMILES: CCCCCCCCCOC(=O)CCCCCCCN(CCCCCCCC(=O)OC(CCCCCCCC)CCCCCCCC)CCCN1C(=O)CCC1=O] |
| I177 | 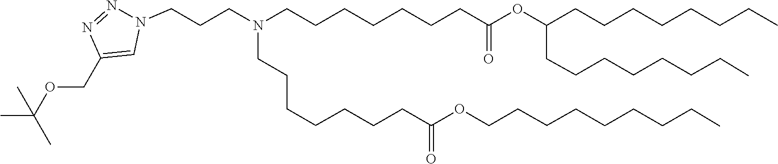 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I178 | 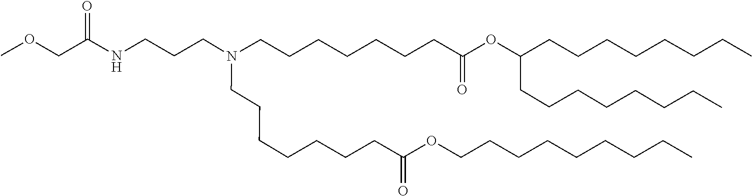 [SMILES: CCCCCCCCCOC(=O)CCCCCCCN(CCCCCCCC(=O)OC(CCCCCCCC)CCCCCCCC)CCCNC(=O)COC] |
| I179 | 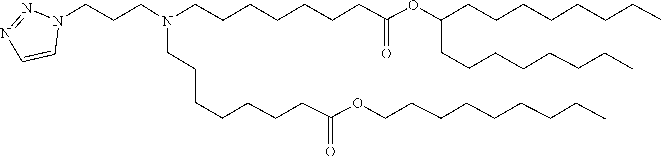 [SMILES: CCCCCCCCCOC(=O)CCCCCCCN(CCCCCCCC(=O)OC(CCCCCCCC)CCCCCCCC)CCCn1ccnn1] |
| I180 | 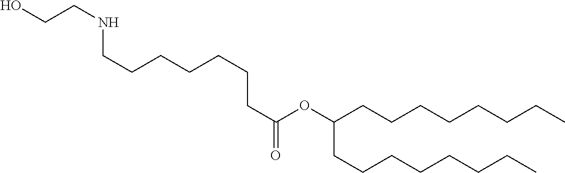 [SMILES: CCCCCCCCC(CCCCCCCC)OC(=O)CCCCCCCNCCO] |
| I181 | 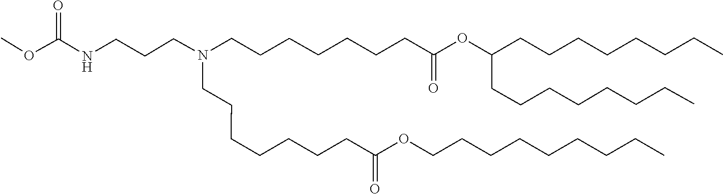 [SMILES: CCCCCCCCCOC(=O)CCCCCCCN(CCCCCCCC(=O)OC(CCCCCCCC)CCCCCCCC)CCCNC(=O)OC] |
| I182 | 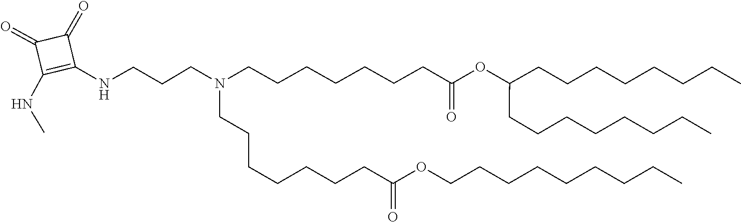 [SMILES: CCCCCCCCCOC(=O)CCCCCCCN(CCCCCCCC(=O)OC(CCCCCCCC)CCCCCCCC)CCCNc1c(NC)c(=O)c1=O] |
| I183 | 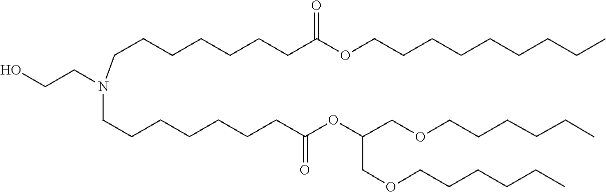 [SMILES: CCCCCCCCCOC(=O)CCCCCCCN(CCO)CCCCCCCC(=O)OC(COCCCCCC)COCCCCCC] |
| I184 | 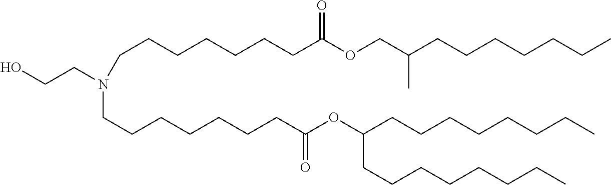 [SMILES: CCCCCCCCC(CCCCCCCC)OC(=O)CCCCCCCN(CCO)CCCCCCCC(=O)OCC(C)CCCCCCC] |
| I185 | 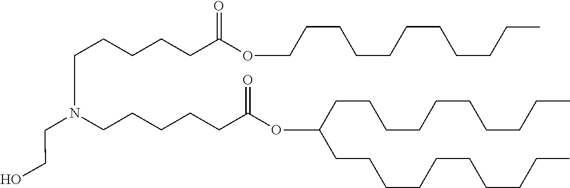 [SMILES: CCCCCCCCCCCOC(=O)CCCCCN(CCO)CCCCCC(=O)OC(CCCCCCCCCC)CCCCCCCCCC] |
| I186 | 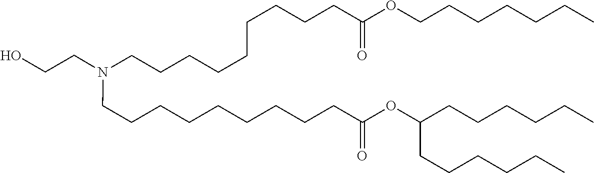 [SMILES: CCCCCCCOC(=O)CCCCCCCCCN(CCO)CCCCCCCCCC(=O)OC(CCCCCC)CCCCCC] |
| I187 | 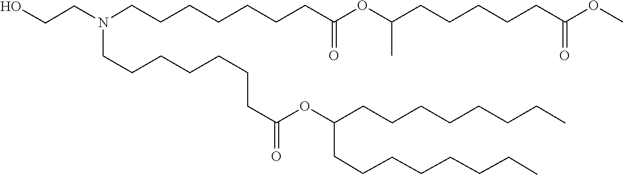 [SMILES: CCCCCCCCC(CCCCCCCC)OC(=O)CCCCCCCN(CCO)CCCCCCCC(=O)OC(C)CCCCCC(=O)OC] |
| I188 | 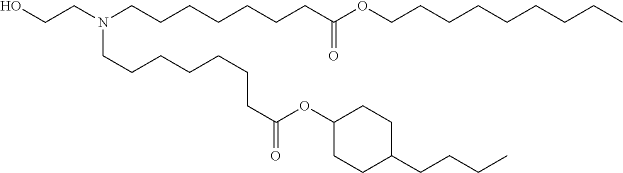 [SMILES: CCCCCCCCCOC(=O)CCCCCCCN(CCO)CCCCCCCC(=O)OC1CCC(CCCC)CC1] |
| I189 | 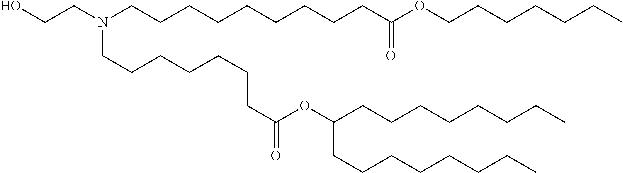 [SMILES: CCCCCCCCC(CCCCCCCC)OC(=O)CCCCCCCN(CCO)CCCCCCCCCC(=O)OCCCCCCC] |
| I190 | 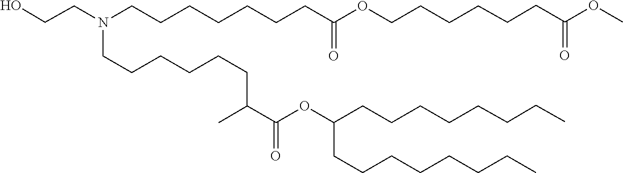 [SMILES: CCCCCCCCC(CCCCCCCC)OC(=O)C(C)CCCCCCN(CCO)CCCCCCCC(=O)OCCCCCCC(=O)OC] |
| I191 | 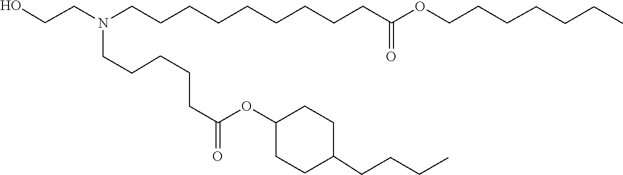 [SMILES: CCCCCCCOC(=O)CCCCCCCCCN(CCO)CCCCCC(=O)OC1CCC(CCCC)CC1] |
| I192 | 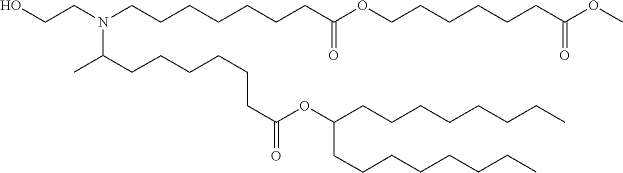 [SMILES: CCCCCCCCC(CCCCCCCC)OC(=O)CCCCCCC(C)N(CCO)CCCCCCCC(=O)OCCCCCCC(=O)OC] |
| I193 | 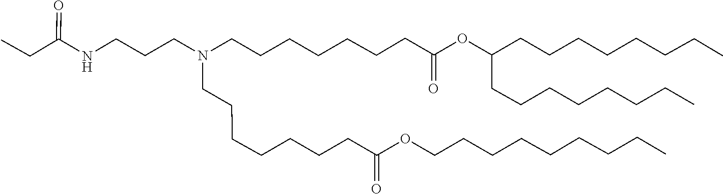 [SMILES: CCCCCCCCCOC(=O)CCCCCCCN(CCCCCCCC(=O)OC(CCCCCCCC)CCCCCCCC)CCCNC(=O)CC] |
| I194 | 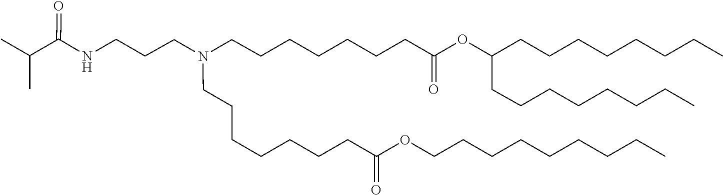 [SMILES: CCCCCCCCCOC(=O)CCCCCCCN(CCCCCCCC(=O)OC(CCCCCCCC)CCCCCCCC)CCCNC(=O)C(C)C] |
| I195 | 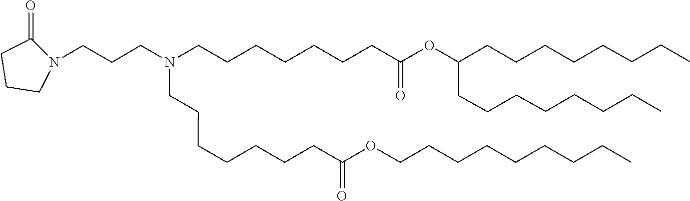 [SMILES: CCCCCCCCCOC(=O)CCCCCCCN(CCCCCCCC(=O)OC(CCCCCCCC)CCCCCCCC)CCCN1CCCC1=O] |
| I196 | 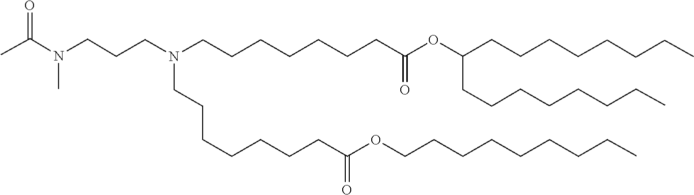 [SMILES: CCCCCCCCCOC(=O)CCCCCCCN(CCCCCCCC(=O)OC(CCCCCCCC)CCCCCCCC)CCCN(C)C(C)=O] |
| I197 | 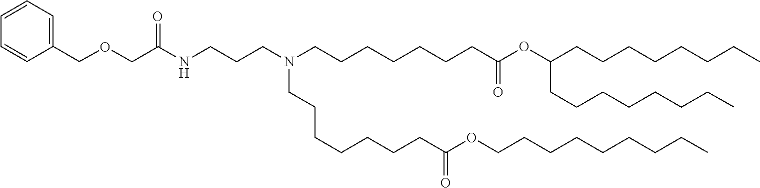 [SMILES: CCCCCCCCCOC(=O)CCCCCCCN(CCCCCCCC(=O)OC(CCCCCCCC)CCCCCCCC)CCCNC(=O)COCc1ccccc1] |
| I198 | 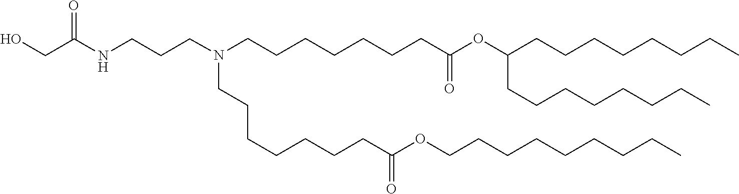 [SMILES: CCCCCCCCCOC(=O)CCCCCCCN(CCCCCCCC(=O)OC(CCCCCCCC)CCCCCCCC)CCCNC(=O)CO] |
| I199 | 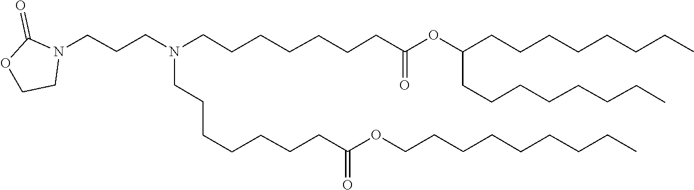 [SMILES: CCCCCCCCCOC(=O)CCCCCCCN(CCCCCCCC(=O)OC(CCCCCCCC)CCCCCCCC)CCCN1CCOC1=O] |
| I200 | 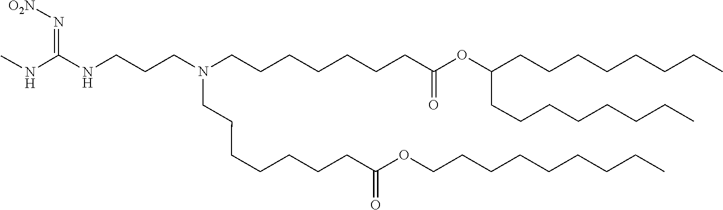 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I201 | 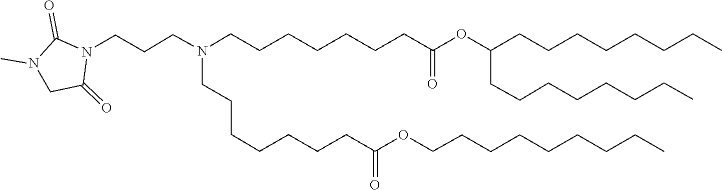 [SMILES: CCCCCCCCCOC(=O)CCCCCCCN(CCCCCCCC(=O)OC(CCCCCCCC)CCCCCCCC)CCCN1C(=O)CN(C)C1=O] |
| I202 | 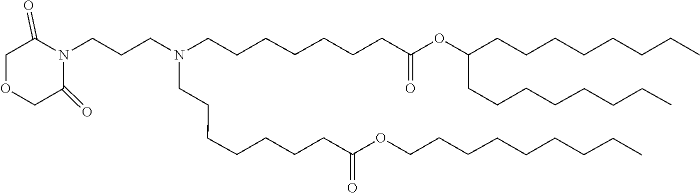 [SMILES: CCCCCCCCCOC(=O)CCCCCCCN(CCCCCCCC(=O)OC(CCCCCCCC)CCCCCCCC)CCCN1C(=O)COCC1=O] |
| I203 | 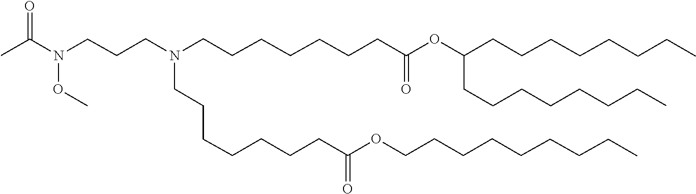 [SMILES: CCCCCCCCCOC(=O)CCCCCCCN(CCCCCCCC(=O)OC(CCCCCCCC)CCCCCCCC)CCCN(OC)C(C)=O] |
| I204 | 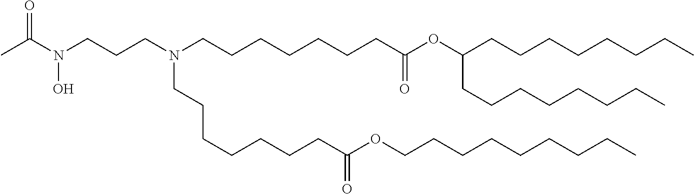 [SMILES: CCCCCCCCCOC(=O)CCCCCCCN(CCCCCCCC(=O)OC(CCCCCCCC)CCCCCCCC)CCCN(O)C(C)=O] |
| I205 | 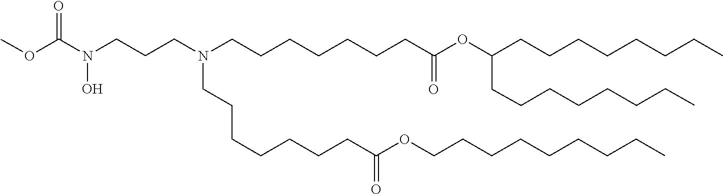 [SMILES: CCCCCCCCCOC(=O)CCCCCCCN(CCCCCCCC(=O)OC(CCCCCCCC)CCCCCCCC)CCCN(O)C(=O)OC] |
| I206 | 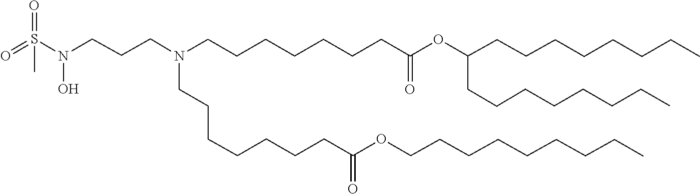 [SMILES: CCCCCCCCCOC(=O)CCCCCCCN(CCCCCCCC(=O)OC(CCCCCCCC)CCCCCCCC)CCCN(O)S(C)(=O)=O] |
| I207 | 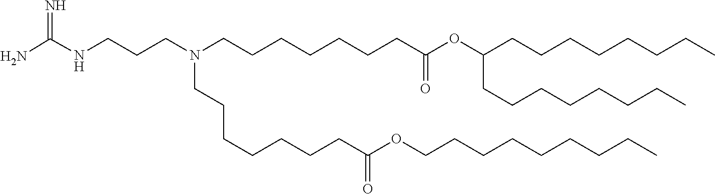 [SMILES: CCCCCCCCCOC(=O)CCCCCCCN(CCCCCCCC(=O)OC(CCCCCCCC)CCCCCCCC)CCCNC(=N)N] |
| I208 | 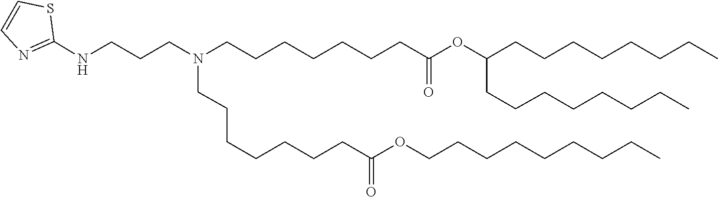 [SMILES: CCCCCCCCCOC(=O)CCCCCCCN(CCCCCCCC(=O)OC(CCCCCCCC)CCCCCCCC)CCCNc1nccs1] |
| I209 | 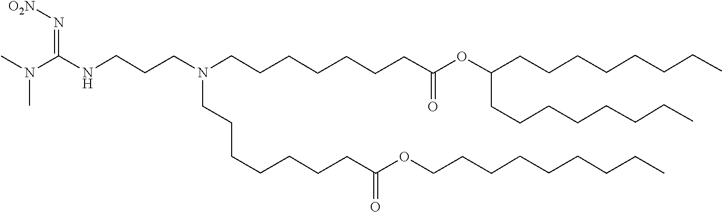 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I210 | 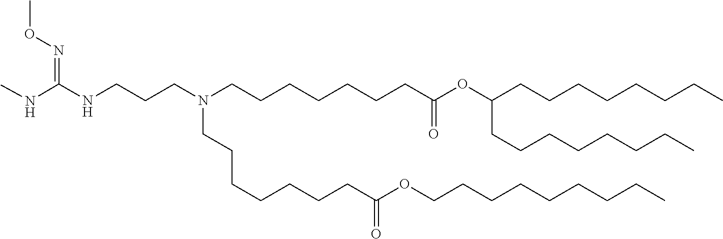 [SMILES: CCCCCCCCCOC(=O)CCCCCCCN(CCCCCCCC(=O)OC(CCCCCCCC)CCCCCCCC)CCCN/C(=N/OC)NC] |
| I211 | 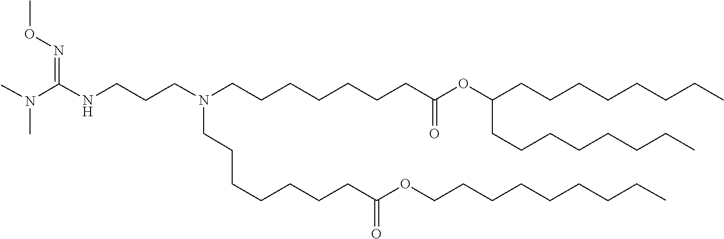 [SMILES: CCCCCCCCCOC(=O)CCCCCCCN(CCCCCCCC(=O)OC(CCCCCCCC)CCCCCCCC)CCCN/C(=N/OC)N(C)C] |
| I212 | 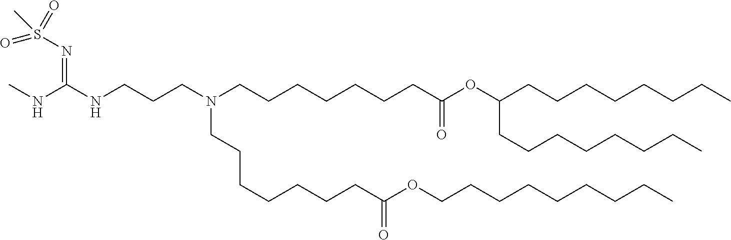 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I213 | 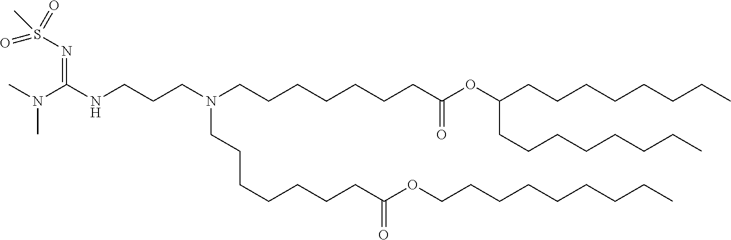 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I214 | 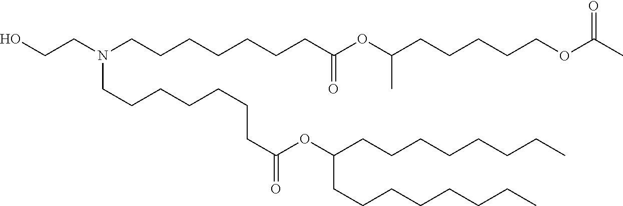 [SMILES: CCCCCCCCC(CCCCCCCC)OC(=O)CCCCCCCN(CCO)CCCCCCCC(=O)OC(C)CCCCCOC(C)=O] |
| I215 | 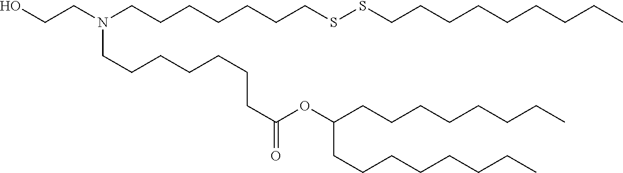 [SMILES: CCCCCCCCCSSCCCCCCCN(CCO)CCCCCCCC(=O)OC(CCCCCCCC)CCCCCCCC] |
| I216 | 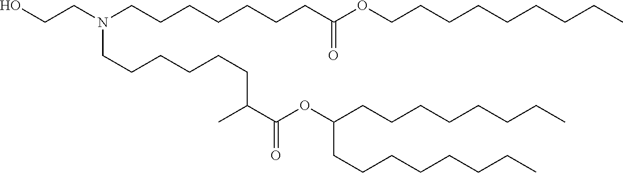 [SMILES: CCCCCCCCCOC(=O)CCCCCCCN(CCO)CCCCCCC(C)C(=O)OC(CCCCCCCC)CCCCCCCC] |
| I217 | 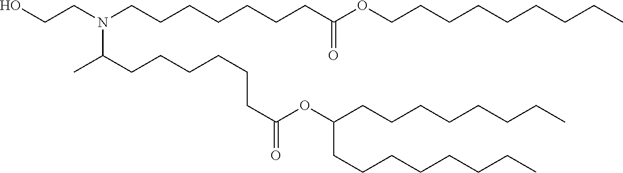 [SMILES: CCCCCCCCCOC(=O)CCCCCCCN(CCO)C(C)CCCCCCC(=O)OC(CCCCCCCC)CCCCCCCC] |
| I218 | 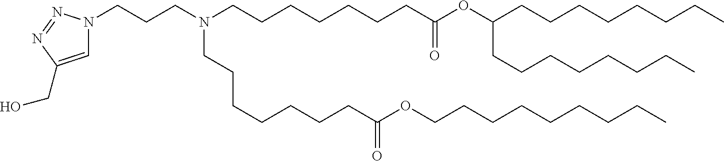 [SMILES: CCCCCCCCCOC(=O)CCCCCCCN(CCCCCCCC(=O)OC(CCCCCCCC)CCCCCCCC)CCCn1cc(CO)nn1] |
| I219 | 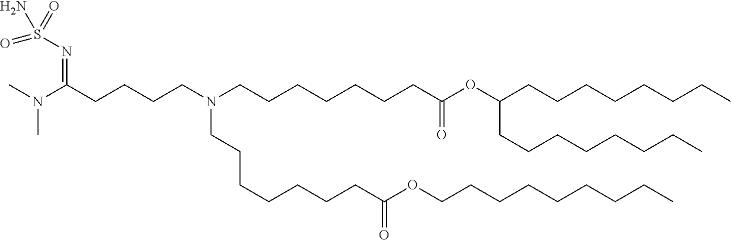 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I220 | 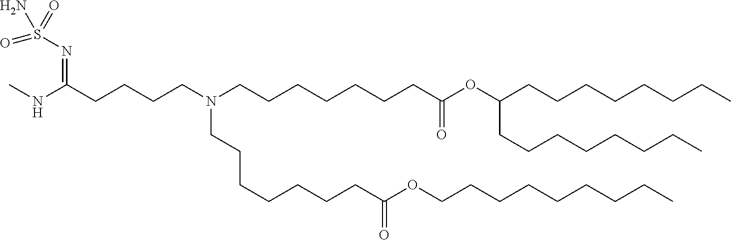 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I221 | 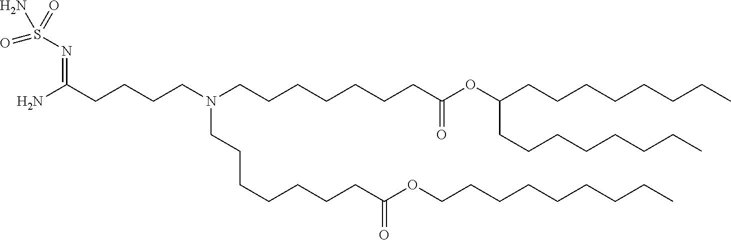 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I222 | 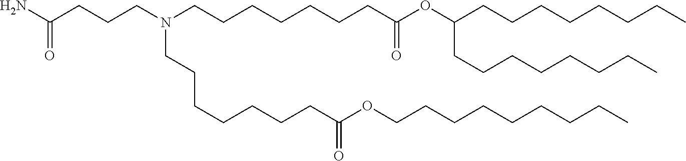 [SMILES: CCCCCCCCCOC(=O)CCCCCCCN(CCCCCCCC(=O)OC(CCCCCCCC)CCCCCCCC)CCCC(N)=O] |
| I223 | 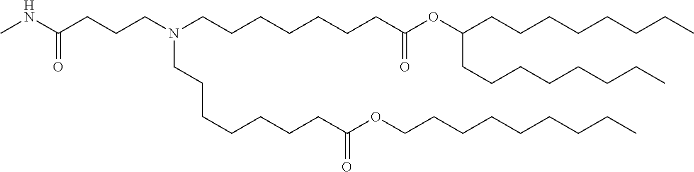 [SMILES: CCCCCCCCCOC(=O)CCCCCCCN(CCCCCCCC(=O)OC(CCCCCCCC)CCCCCCCC)CCCC(=O)NC] |
| I224 | 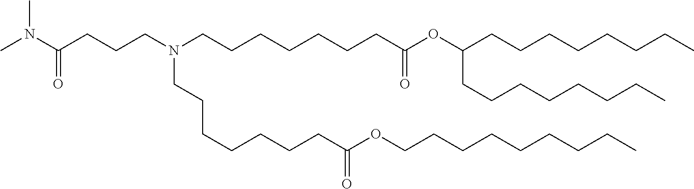 [SMILES: CCCCCCCCCOC(=O)CCCCCCCN(CCCCCCCC(=O)OC(CCCCCCCC)CCCCCCCC)CCCC(=O)N(C)C] |
| I225 | 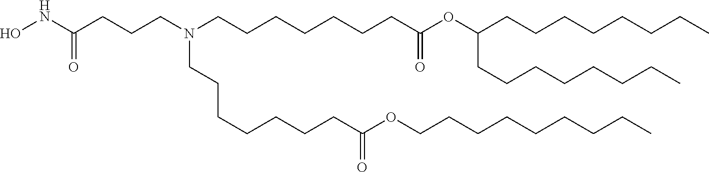 [SMILES: CCCCCCCCCOC(=O)CCCCCCCN(CCCCCCCC(=O)OC(CCCCCCCC)CCCCCCCC)CCCC(=O)NO] |
| I226 | 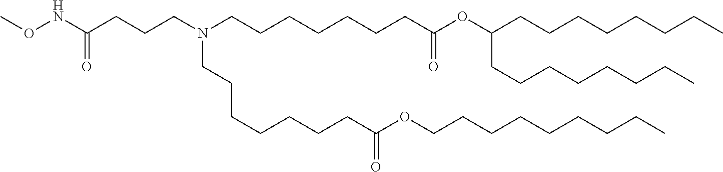 [SMILES: CCCCCCCCCOC(=O)CCCCCCCN(CCCCCCCC(=O)OC(CCCCCCCC)CCCCCCCC)CCCC(=O)NOC] |
| I227 | 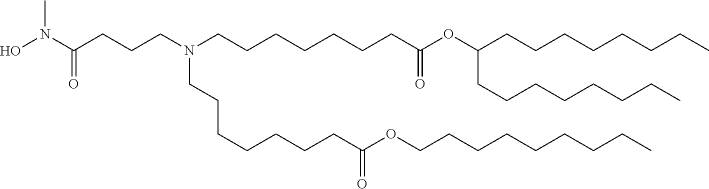 [SMILES: CCCCCCCCCOC(=O)CCCCCCCN(CCCCCCCC(=O)OC(CCCCCCCC)CCCCCCCC)CCCC(=O)N(C)O] |
| I228 | 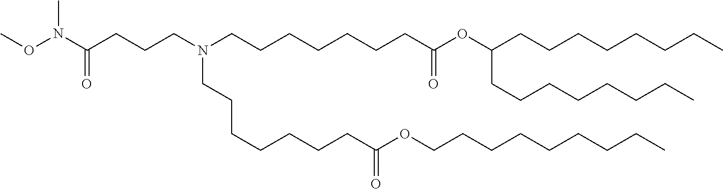 [SMILES: CCCCCCCCCOC(=O)CCCCCCCN(CCCCCCCC(=O)OC(CCCCCCCC)CCCCCCCC)CCCC(=O)N(C)OC] |
| I229 | 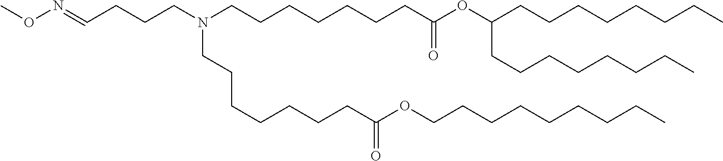 [SMILES: CCCCCCCCCOC(=O)CCCCCCCN(CCC/C=N/OC)CCCCCCCC(=O)OC(CCCCCCCC)CCCCCCCC] |
| I230 | 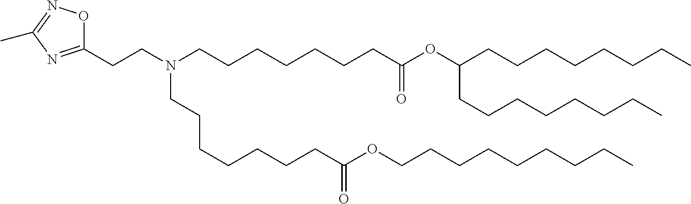 [SMILES: CCCCCCCCCOC(=O)CCCCCCCN(CCCCCCCC(=O)OC(CCCCCCCC)CCCCCCCC)CCc1nc(C)no1] |
| I231 | 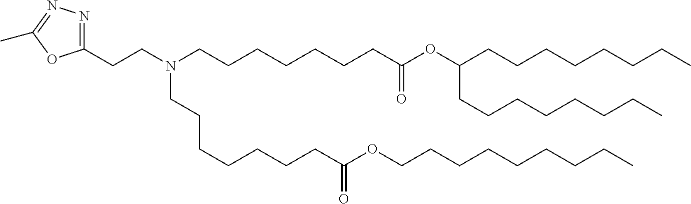 [SMILES: CCCCCCCCCOC(=O)CCCCCCCN(CCCCCCCC(=O)OC(CCCCCCCC)CCCCCCCC)CCc1nnc(C)o1] |
| I232 | 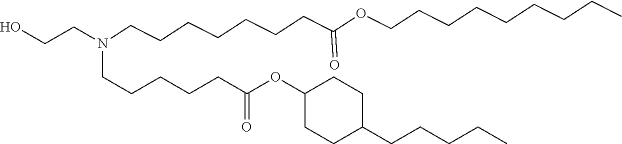 [SMILES: CCCCCCCCCOC(=O)CCCCCCCN(CCO)CCCCCC(=O)OC1CCC(CCCCC)CC1] |
| I233 | 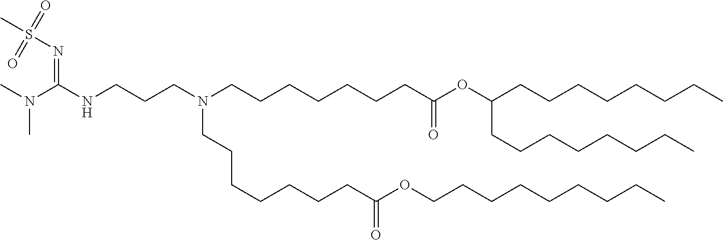 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I234 | 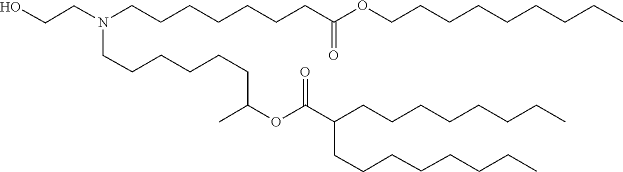 [SMILES: CCCCCCCCCOC(=O)CCCCCCCN(CCO)CCCCCCC(C)OC(=O)C(CCCCCCCC)CCCCCCCC] |
| I235 | 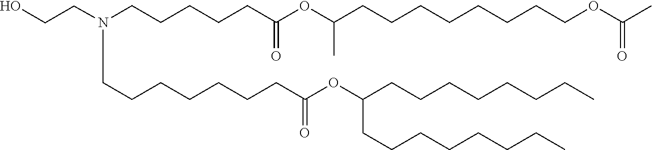 [SMILES: CCCCCCCCC(CCCCCCCC)OC(=O)CCCCCCCN(CCO)CCCCCC(=O)OC(C)CCCCCCCCOC(C)=O] |
| I236 | 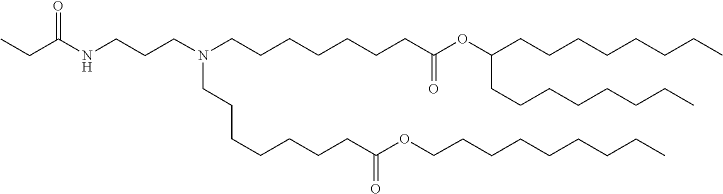 [SMILES: CCCCCCCCCOC(=O)CCCCCCCN(CCCCCCCC(=O)OC(CCCCCCCC)CCCCCCCC)CCCNC(=O)CC] |
| I237 | 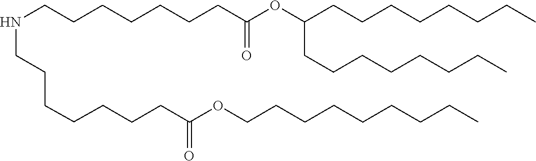 [SMILES: CCCCCCCCCOC(=O)CCCCCCCNCCCCCCCC(=O)OC(CCCCCCCC)CCCCCCCC] |
| I238 | 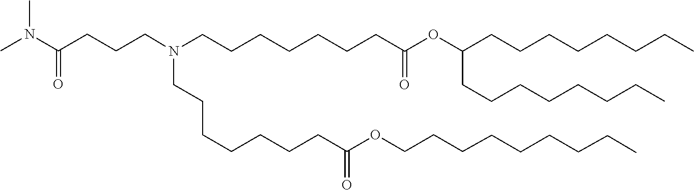 [SMILES: CCCCCCCCCOC(=O)CCCCCCCN(CCCCCCCC(=O)OC(CCCCCCCC)CCCCCCCC)CCCC(=O)N(C)C] |
| I239 | 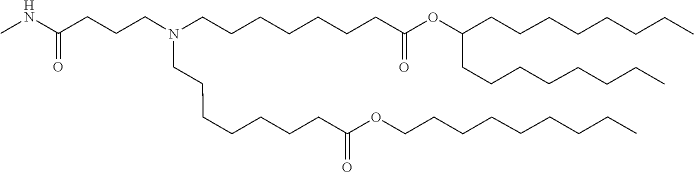 [SMILES: CCCCCCCCCOC(=O)CCCCCCCN(CCCCCCCC(=O)OC(CCCCCCCC)CCCCCCCC)CCCC(=O)NC] |
| I240 | 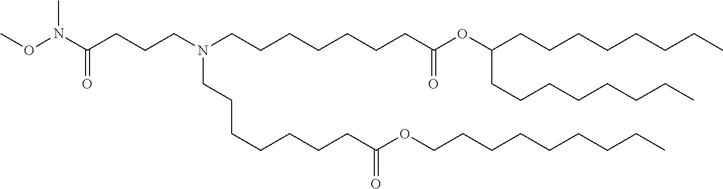 [SMILES: CCCCCCCCCOC(=O)CCCCCCCN(CCCCCCCC(=O)OC(CCCCCCCC)CCCCCCCC)CCCC(=O)N(C)OC] |
| I241 | 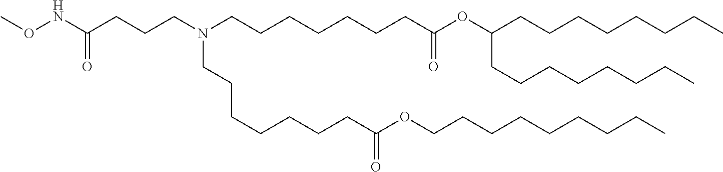 [SMILES: CCCCCCCCCOC(=O)CCCCCCCN(CCCCCCCC(=O)OC(CCCCCCCC)CCCCCCCC)CCCC(=O)NOC] |
| I242 | 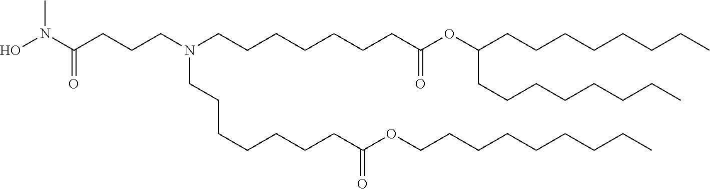 [SMILES: CCCCCCCCCOC(=O)CCCCCCCN(CCCCCCCC(=O)OC(CCCCCCCC)CCCCCCCC)CCCC(=O)N(C)O] |
| I243 | 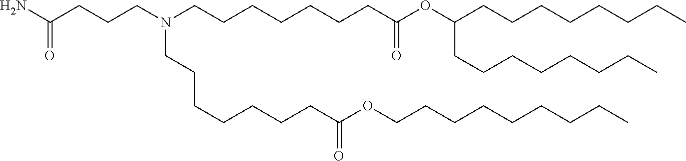 [SMILES: CCCCCCCCCOC(=O)CCCCCCCN(CCCCCCCC(=O)OC(CCCCCCCC)CCCCCCCC)CCCC(N)=O] |
| I244 | 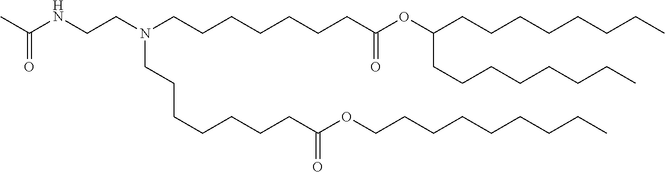 [SMILES: CCCCCCCCCOC(=O)CCCCCCCN(CCCCCCCC(=O)OC(CCCCCCCC)CCCCCCCC)CCNC(C)=O] |
| I245 | 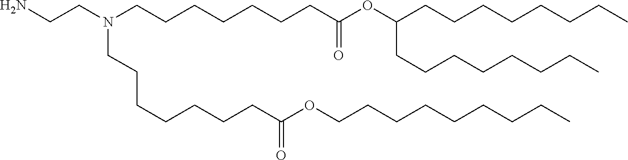 [SMILES: CCCCCCCCCOC(=O)CCCCCCCN(CCN)CCCCCCCC(=O)OC(CCCCCCCC)CCCCCCCC] |
| I246 | 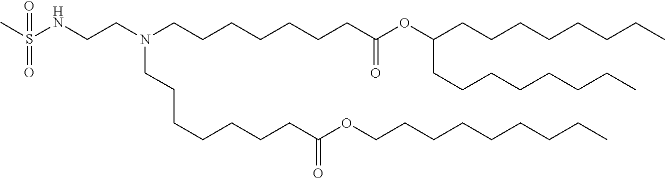 [SMILES: CCCCCCCCCOC(=O)CCCCCCCN(CCCCCCCC(=O)OC(CCCCCCCC)CCCCCCCC)CCNS(C)(=O)=O] |
| I247 | 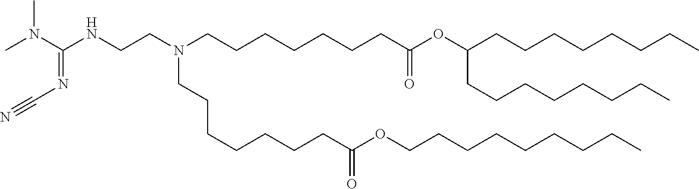 [SMILES: CCCCCCCCCOC(=O)CCCCCCCN(CCCCCCCC(=O)OC(CCCCCCCC)CCCCCCCC)CCN/C(=N/C#N)N(C)C] |
| I248 | 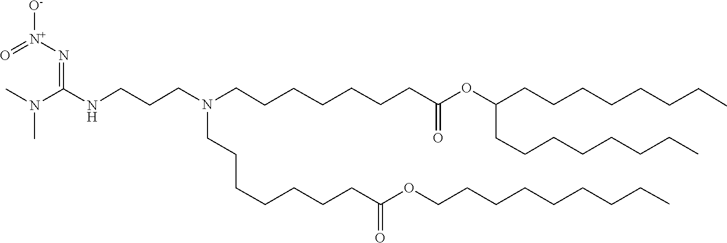 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I249 | 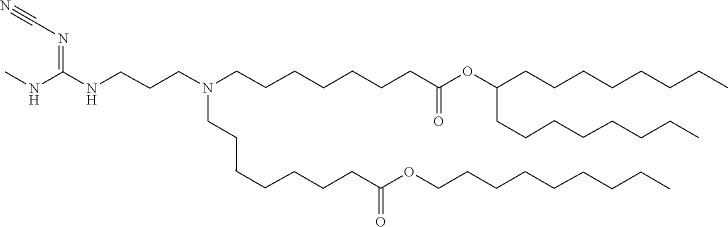 [SMILES: CCCCCCCCCOC(=O)CCCCCCCN(CCCCCCCC(=O)OC(CCCCCCCC)CCCCCCCC)CCCN/C(=N/C#N)NC] |
| I250 | 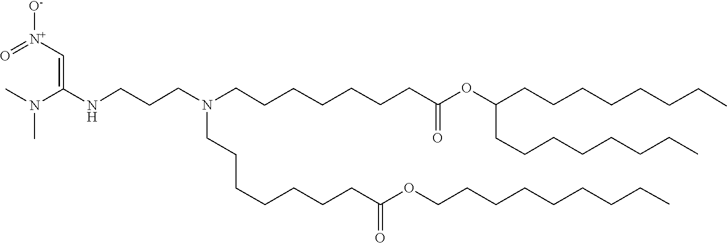 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I251 | 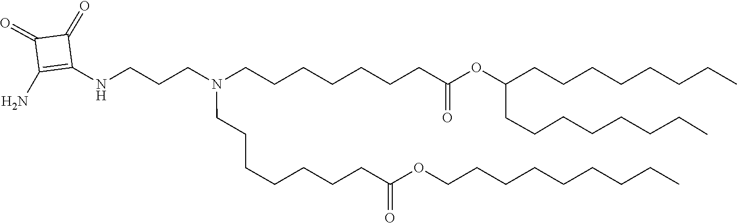 [SMILES: CCCCCCCCCOC(=O)CCCCCCCN(CCCCCCCC(=O)OC(CCCCCCCC)CCCCCCCC)CCCNc1c(N)c(=O)c1=O] |
| I252 | 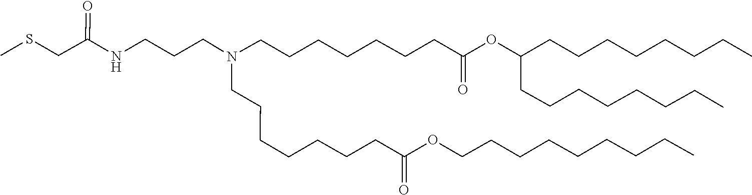 [SMILES: CCCCCCCCCOC(=O)CCCCCCCN(CCCCCCCC(=O)OC(CCCCCCCC)CCCCCCCC)CCCNC(=O)CSC] |
| I253 | 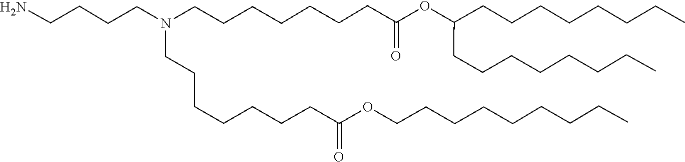 [SMILES: CCCCCCCCCOC(=O)CCCCCCCN(CCCCN)CCCCCCCC(=O)OC(CCCCCCCC)CCCCCCCC] |
| I254 | 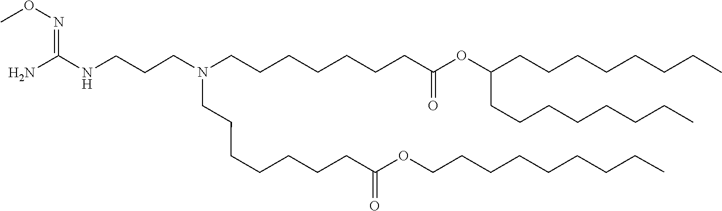 [SMILES: CCCCCCCCCOC(=O)CCCCCCCN(CCCCCCCC(=O)OC(CCCCCCCC)CCCCCCCC)CCCN/C(N)=N/OC] |
| I255 | 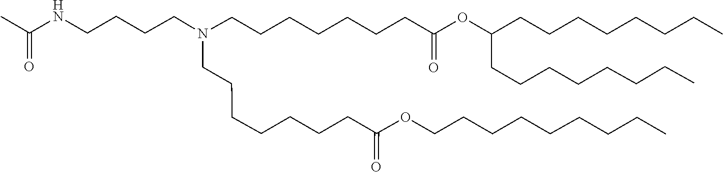 [SMILES: CCCCCCCCCOC(=O)CCCCCCCN(CCCCCCCC(=O)OC(CCCCCCCC)CCCCCCCC)CCCCNC(C)=O] |
| I256 | 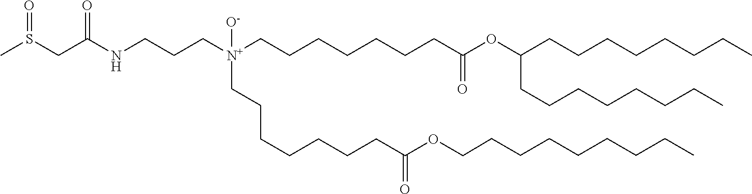 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I257 | 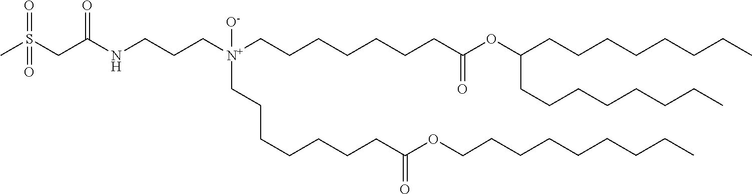 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I258 | 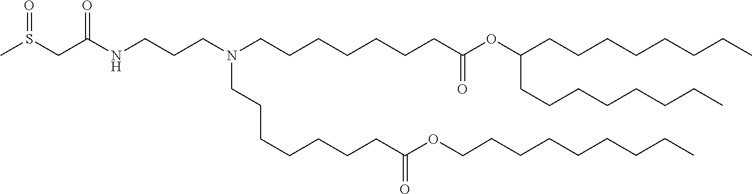 [SMILES: CCCCCCCCCOC(=O)CCCCCCCN(CCCCCCCC(=O)OC(CCCCCCCC)CCCCCCCC)CCCNC(=O)CS(C)=O] |
| I259 | 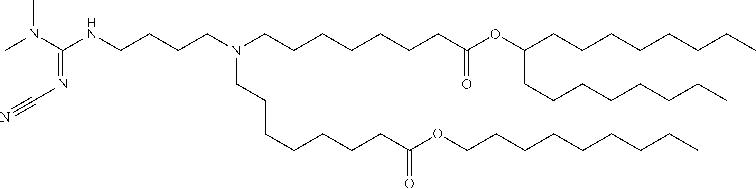 [SMILES: CCCCCCCCCOC(=O)CCCCCCCN(CCCCCCCC(=O)OC(CCCCCCCC)CCCCCCCC)CCCCN/C(=N/C#N)N(C)C] |
| I260 | 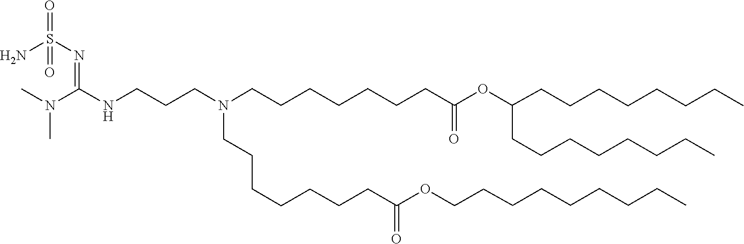 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I261 | 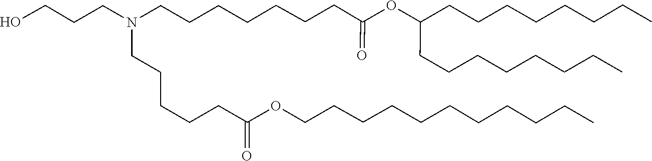 [SMILES: CCCCCCCCCCCOC(=O)CCCCCN(CCCO)CCCCCCCC(=O)OC(CCCCCCCC)CCCCCCCC] |
| I262 | 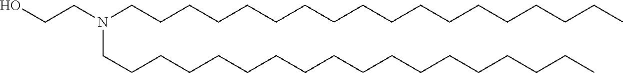 [SMILES: CCCCCCCCCCCCCCCCCCN(CCO)CCCCCCCCCCCCCCCCCC] |
| I263 | 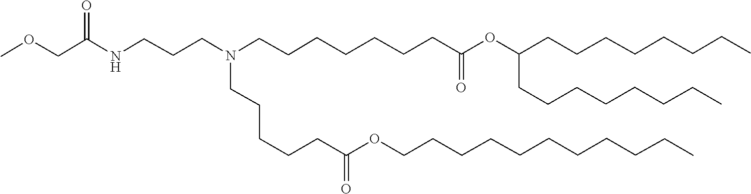 [SMILES: CCCCCCCCCCCOC(=O)CCCCCN(CCCCCCCC(=O)OC(CCCCCCCC)CCCCCCCC)CCCNC(=O)COC] |
| I264 | 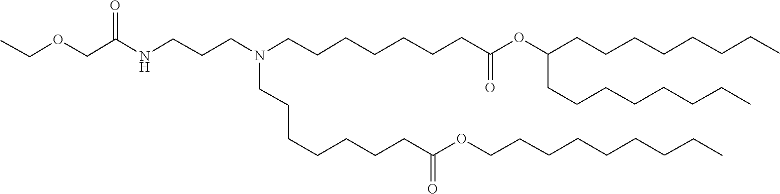 [SMILES: CCCCCCCCCOC(=O)CCCCCCCN(CCCCCCCC(=O)OC(CCCCCCCC)CCCCCCCC)CCCNC(=O)COCC] |
| I265 | 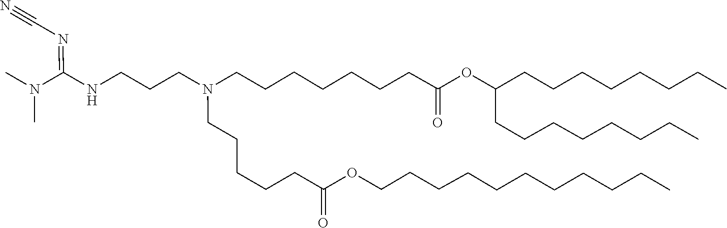 [SMILES: CCCCCCCCCCCOC(=O)CCCCCN(CCCCCCCC(=O)OC(CCCCCCCC)CCCCCCCC)CCCN/C(=N/C#N)N(C)C] |
| I266 | 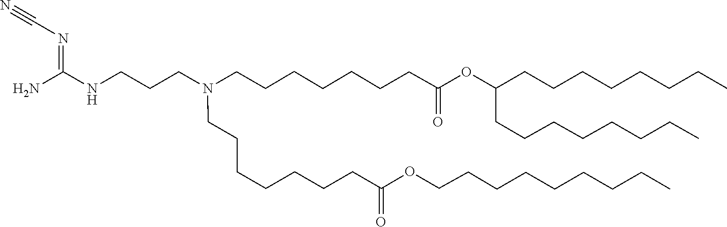 [SMILES: CCCCCCCCCOC(=O)CCCCCCCN(CCCCCCCC(=O)OC(CCCCCCCC)CCCCCCCC)CCCN/C(N)=N/C#N] |
| I267 | 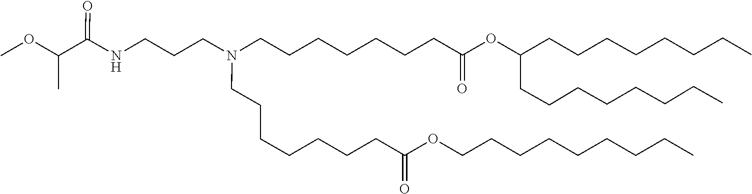 [SMILES: CCCCCCCCCOC(=O)CCCCCCCN(CCCCCCCC(=O)OC(CCCCCCCC)CCCCCCCC)CCCNC(=O)C(C)OC] |
| I268 | 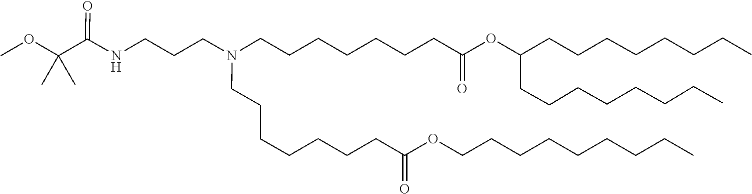 [SMILES: CCCCCCCCCOC(=O)CCCCCCCN(CCCCCCCC(=O)OC(CCCCCCCC)CCCCCCCC)CCCNC(=O)C(C)(C)OC] |
| I269 | 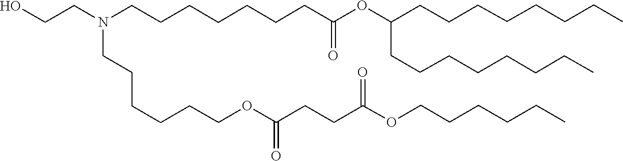 [SMILES: CCCCCCCCC(CCCCCCCC)OC(=O)CCCCCCCN(CCO)CCCCCCOC(=O)CCC(=O)OCCCCCC] |
| I270 | 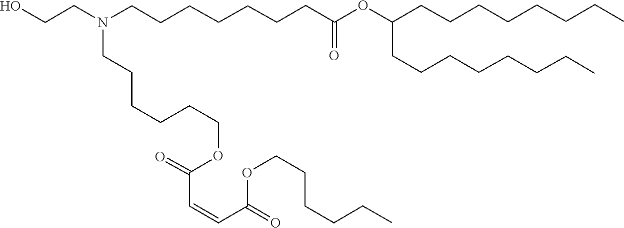 [SMILES: CCCCCCCCC(CCCCCCCC)OC(=O)CCCCCCCN(CCO)CCCCCCOC(=O)/C=C\C(=O)OCCCCCC] |
| I271 | 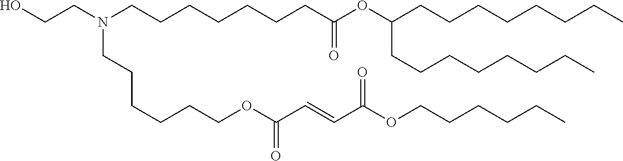 [SMILES: CCCCCCCCC(CCCCCCCC)OC(=O)CCCCCCCN(CCO)CCCCCCOC(=O)/C=C/C(=O)OCCCCCC] |
| I272 | 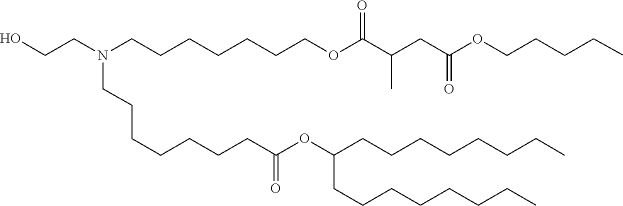 [SMILES: CCCCCCCCC(CCCCCCCC)OC(=O)CCCCCCCN(CCO)CCCCCCCOC(=O)C(C)CC(=O)OCCCCC] |
| I273 | 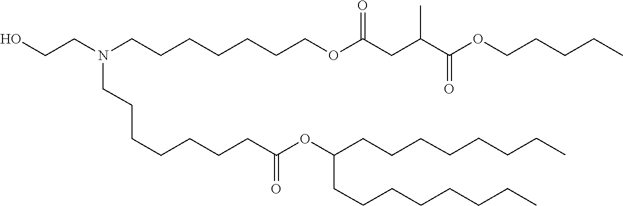 [SMILES: CCCCCCCCC(CCCCCCCC)OC(=O)CCCCCCCN(CCO)CCCCCCCOC(=O)CC(C)C(=O)OCCCCC] |
| I274 | 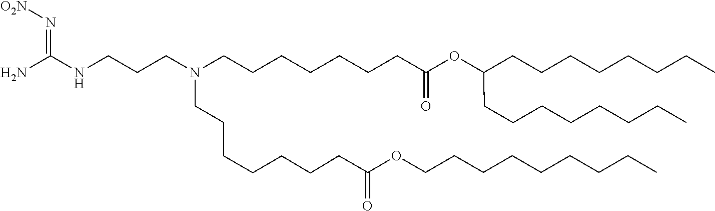 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I275 | 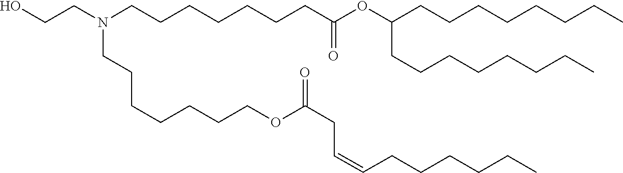 [SMILES: CCCCCC/C=C\CC(=O)OCCCCCCCN(CCO)CCCCCCCC(=O)OC(CCCCCCCC)CCCCCCCC] |
| I276 | 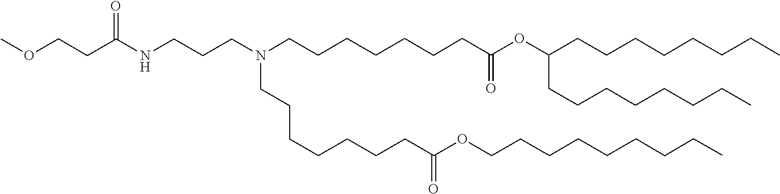 [SMILES: CCCCCCCCCOC(=O)CCCCCCCN(CCCCCCCC(=O)OC(CCCCCCCC)CCCCCCCC)CCCNC(=O)CCOC] |
| I277 | 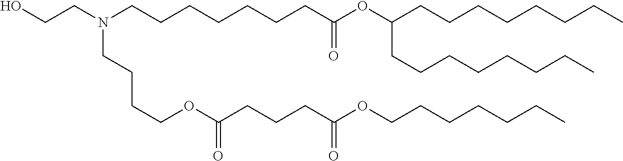 [SMILES: CCCCCCCCC(CCCCCCCC)OC(=O)CCCCCCCN(CCO)CCCCOC(=O)CCCC(=O)OCCCCCCC] |
| I278 | 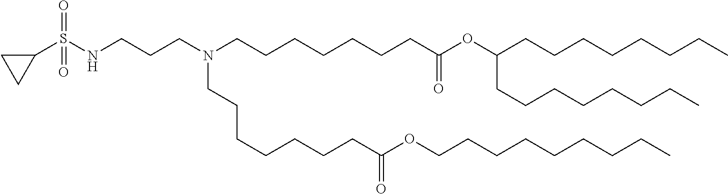 [SMILES: CCCCCCCCCOC(=O)CCCCCCCN(CCCCCCCC(=O)OC(CCCCCCCC)CCCCCCCC)CCCNS(=O)(=O)C1CC1] |
| I279 | 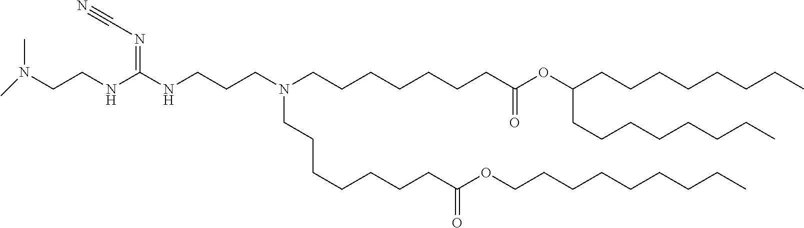 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I280 | 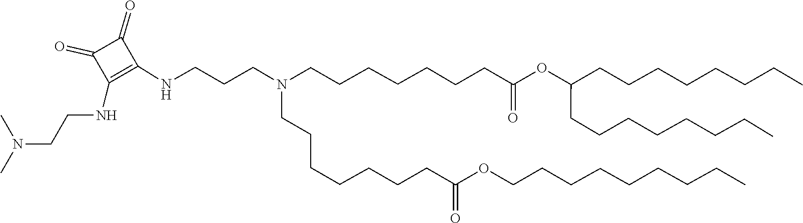 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I281 | 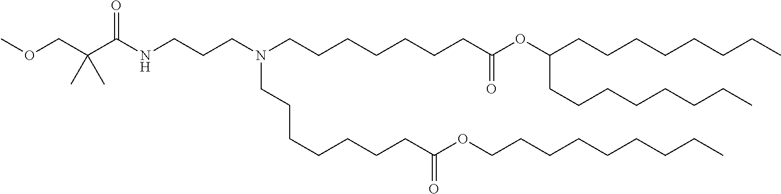 [SMILES: CCCCCCCCCOC(=O)CCCCCCCN(CCCCCCCC(=O)OC(CCCCCCCC)CCCCCCCC)CCCNC(=O)C(C)(C)COC] |
| I282 | 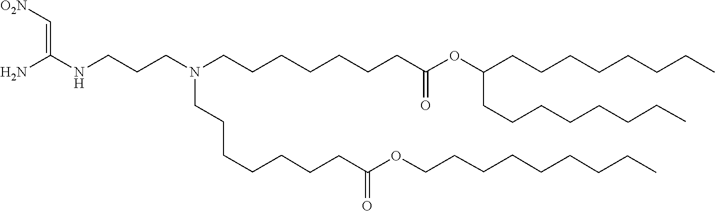 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I283 | 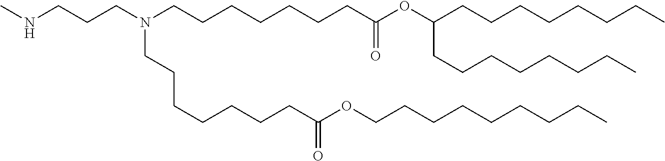 [SMILES: CCCCCCCCCOC(=O)CCCCCCCN(CCCCCCCC(=O)OC(CCCCCCCC)CCCCCCCC)CCCNC] |
| I284 | 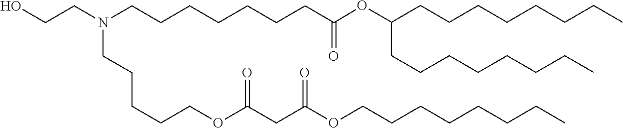 [SMILES: CCCCCCCCOC(=O)CC(=O)OCCCCCN(CCO)CCCCCCCC(=O)OC(CCCCCCCC)CCCCCCCC] |
| I285 | 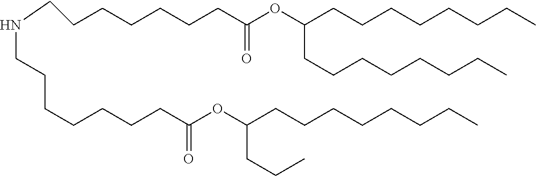 [SMILES: CCCCCCCCC(CCC)OC(=O)CCCCCCCNCCCCCCCC(=O)OC(CCCCCCCC)CCCCCCCC] |
| I286 | 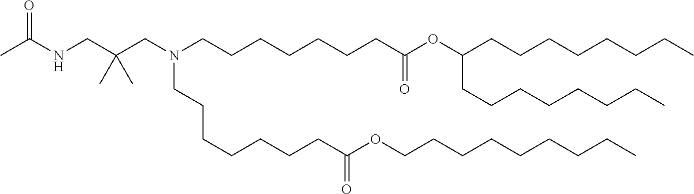 [SMILES: CCCCCCCCCOC(=O)CCCCCCCN(CCCCCCCC(=O)OC(CCCCCCCC)CCCCCCCC)CC(C)(C)CNC(C)=O] |
| I287 | 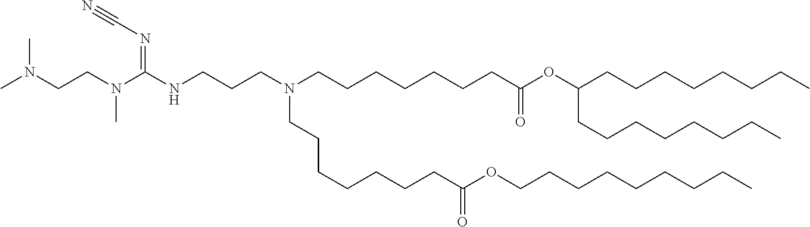 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I288 | 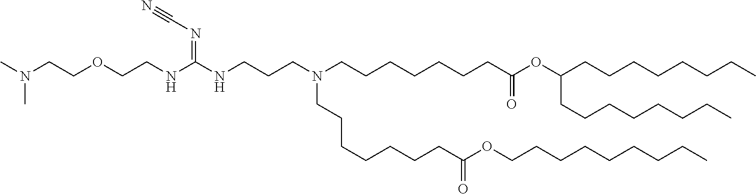 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I289 | 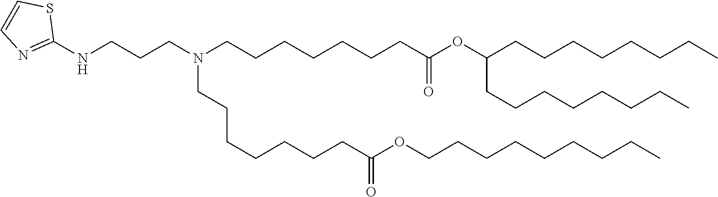 [SMILES: CCCCCCCCCOC(=O)CCCCCCCN(CCCCCCCC(=O)OC(CCCCCCCC)CCCCCCCC)CCCNc1nccs1] |
| I290 | 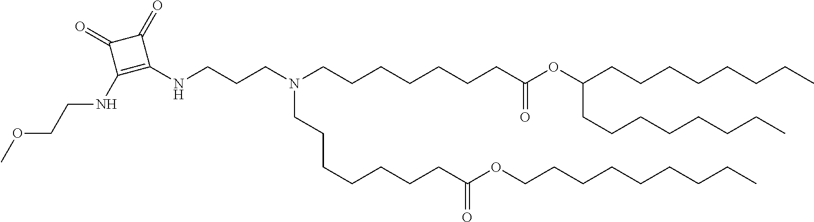 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I291 | 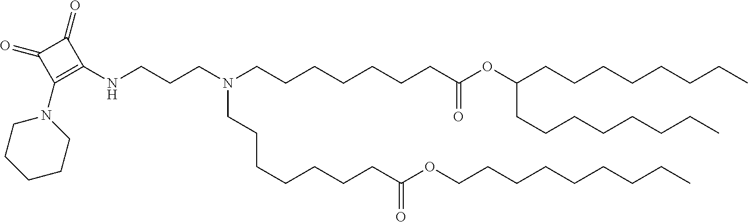 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I292 | 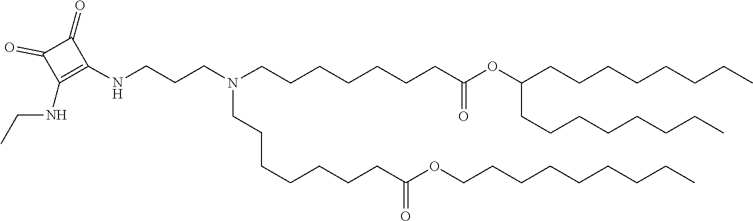 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I293 | 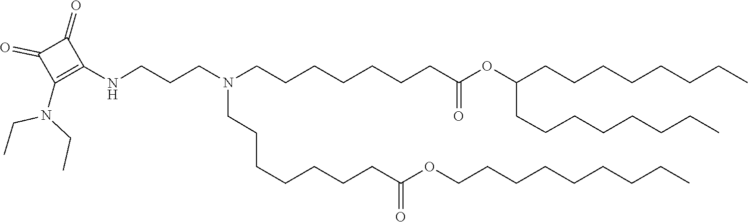 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I294 | 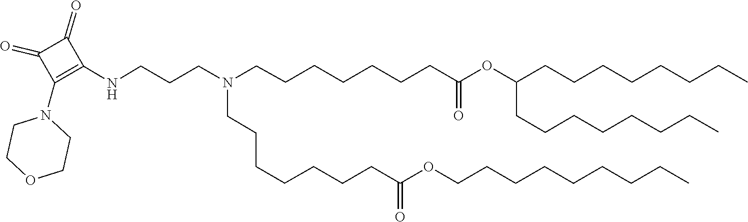 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I295 | 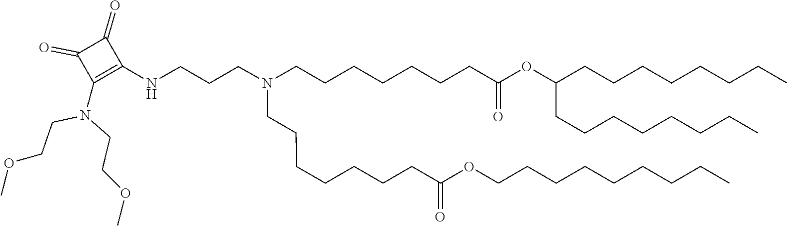 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I296 | 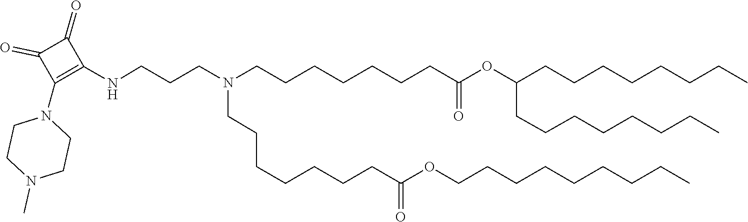 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I297 | 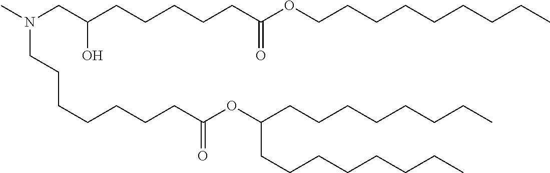 [SMILES: CCCCCCCCCOC(=O)CCCCCC(O)CN(C)CCCCCCCC(=O)OC(CCCCCCCC)CCCCCCCC] |
| I298 | 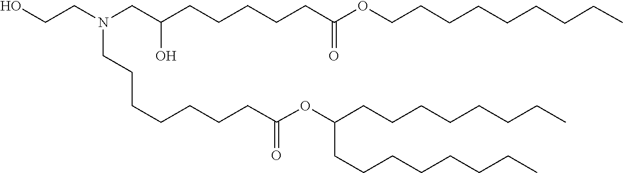 [SMILES: CCCCCCCCCOC(=O)CCCCCC(O)CN(CCO)CCCCCCCC(=O)OC(CCCCCCCC)CCCCCCCC] |
| I299 | 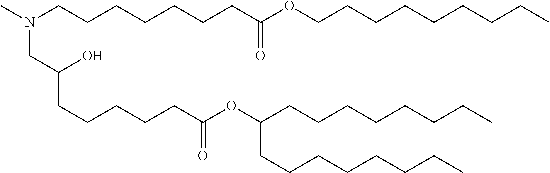 [SMILES: CCCCCCCCCOC(=O)CCCCCCCN(C)CC(O)CCCCCC(=O)OC(CCCCCCCC)CCCCCCCC] |
| I300 | 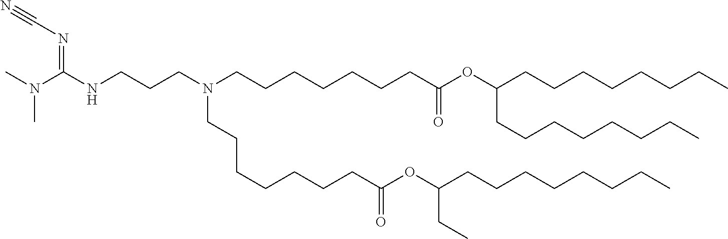 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I301 | 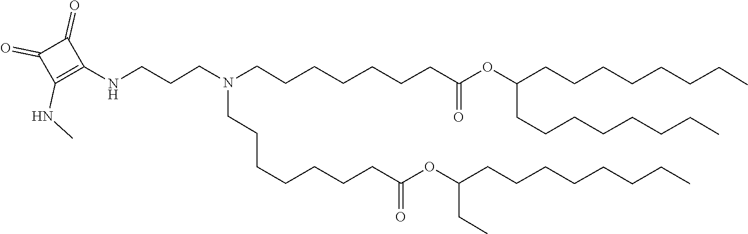 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I302 | 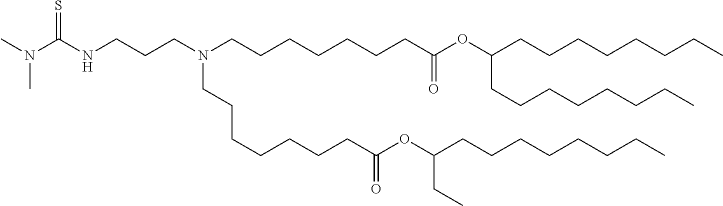 [SMILES: CCCCCCCCC(CC)OC(=O)CCCCCCCN(CCCCCCCC(=O)OC(CCCCCCCC)CCCCCCCC)CCCNC(=S)N(C)C] |
| I303 | 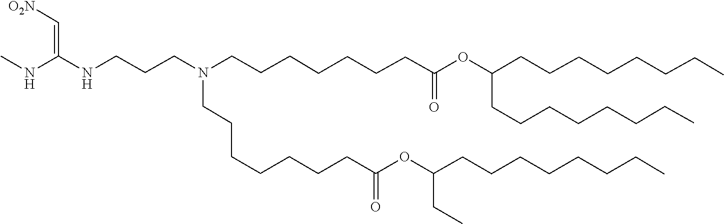 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I304 | 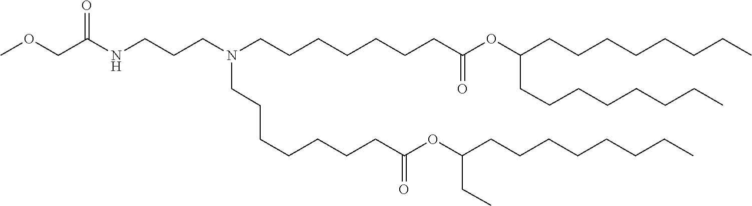 [SMILES: CCCCCCCCC(CC)OC(=O)CCCCCCCN(CCCCCCCC(=O)OC(CCCCCCCC)CCCCCCCC)CCCNC(=O)COC] |
| I305 | 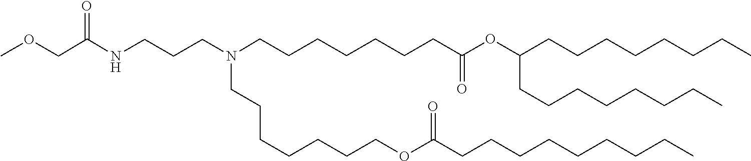 [SMILES: CCCCCCCCCC(=O)OCCCCCCCN(CCCCCCCC(=O)OC(CCCCCCCC)CCCCCCCC)CCCNC(=O)COC] |
| I306 | 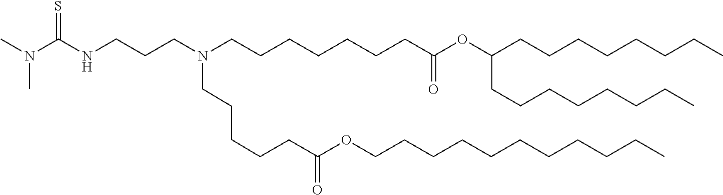 [SMILES: CCCCCCCCCCCOC(=O)CCCCCN(CCCCCCCC(=O)OC(CCCCCCCC)CCCCCCCC)CCCNC(=S)N(C)C] |
| I307 | 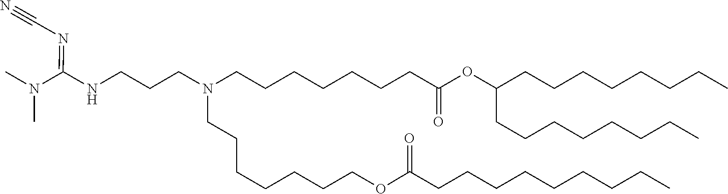 [SMILES: CCCCCCCCCC(=O)OCCCCCCCN(CCCCCCCC(=O)OC(CCCCCCCC)CCCCCCCC)CCCN/C(=N/C#N)N(C)C] |
| I308 | 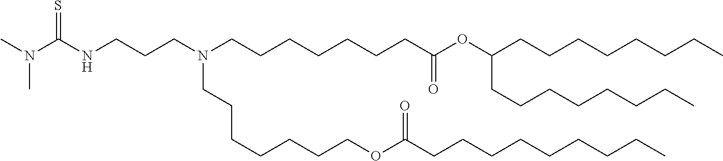 [SMILES: CCCCCCCCCC(=O)OCCCCCCCN(CCCCCCCC(=O)OC(CCCCCCCC)CCCCCCCC)CCCNC(=S)N(C)C] |
| I309 | 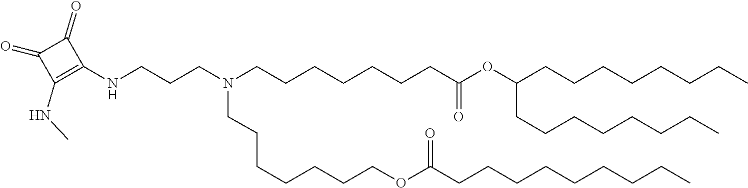 [SMILES: CCCCCCCCCC(=O)OCCCCCCCN(CCCCCCCC(=O)OC(CCCCCCCC)CCCCCCCC)CCCNc1c(NC)c(=O)c1=O] |
| I310 | 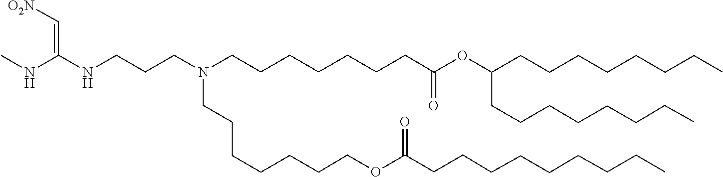 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I311 | 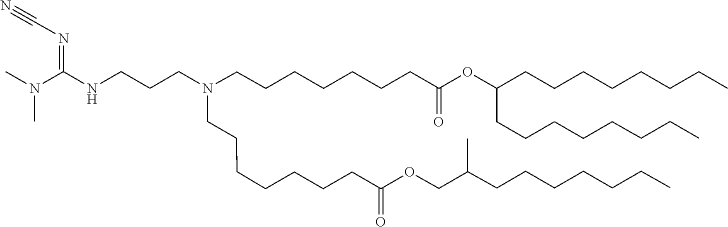 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I312 | 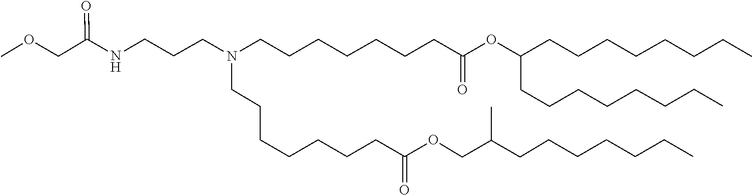 [SMILES: CCCCCCCCC(CCCCCCCC)OC(=O)CCCCCCCN(CCCCCCCC(=O)OCC(C)CCCCCCC)CCCNC(=O)COC] |
| I313 | 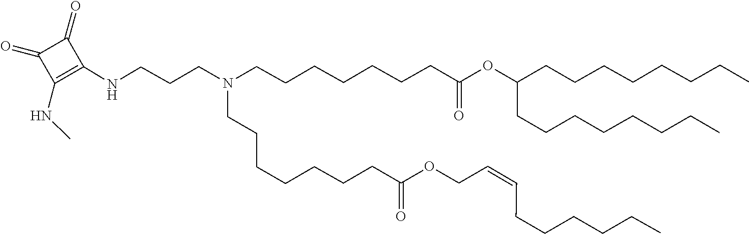 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I314 | 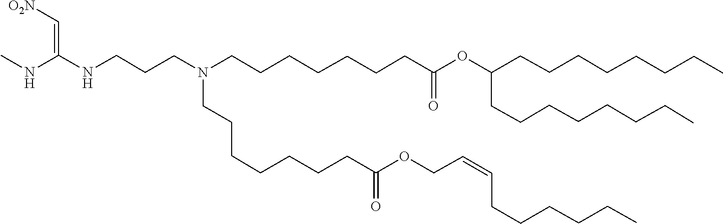 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I315 | 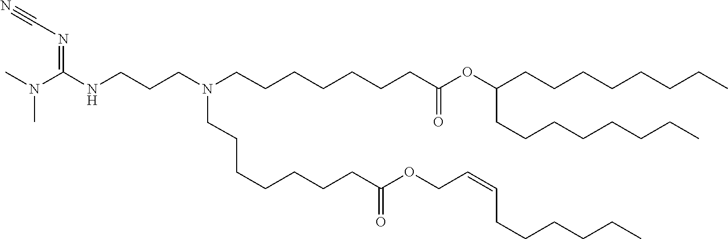 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I316 | 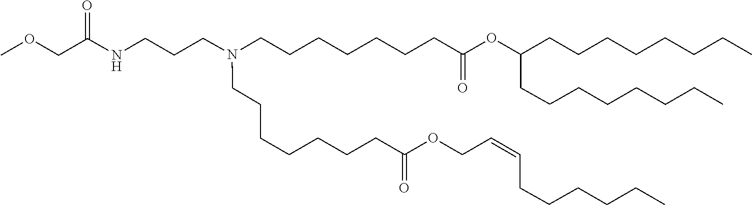 [SMILES: CCCCCC/C=C\COC(=O)CCCCCCCN(CCCCCCCC(=O)OC(CCCCCCCC)CCCCCCCC)CCCNC(=O)COC] |
| I317 | 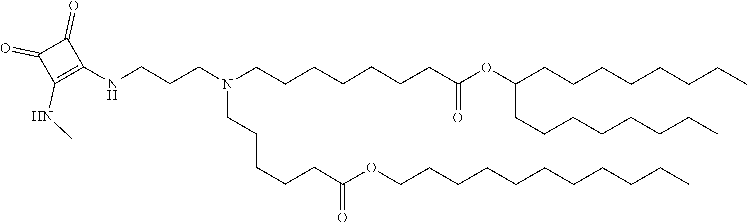 [SMILES: CCCCCCCCCCCOC(=O)CCCCCN(CCCCCCCC(=O)OC(CCCCCCCC)CCCCCCCC)CCCNc1c(NC)c(=O)c1=O] |
| I318 | 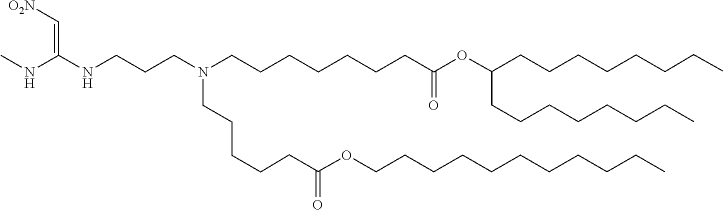 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I319 | 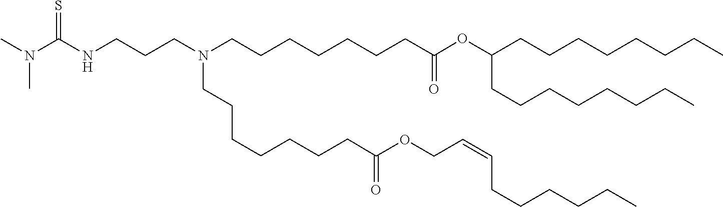 [SMILES: CCCCCC/C=C\COC(=O)CCCCCCCN(CCCCCCCC(=O)OC(CCCCCCCC)CCCCCCCC)CCCNC(=S)N(C)C] |
| I320 | 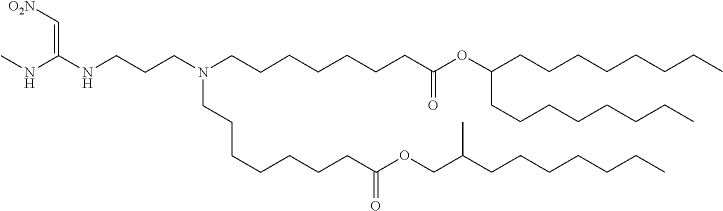 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I321 | 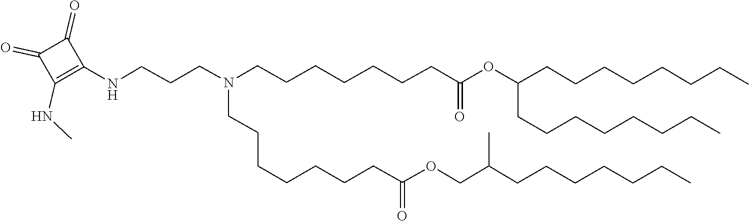 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I322 | 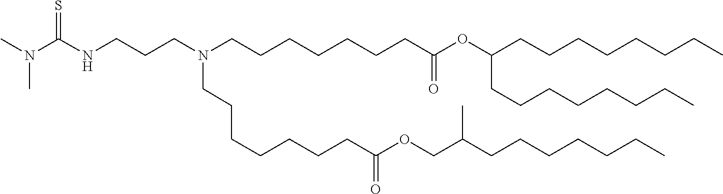 [SMILES: CCCCCCCCC(CCCCCCCC)OC(=O)CCCCCCCN(CCCCCCCC(=O)OCC(C)CCCCCCC)CCCNC(=S)N(C)C] |
| I323 | 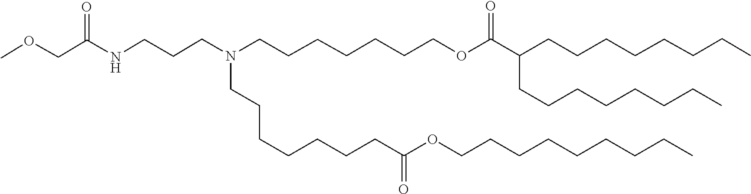 [SMILES: CCCCCCCCCOC(=O)CCCCCCCN(CCCCCCCOC(=O)C(CCCCCCCC)CCCCCCCC)CCCNC(=O)COC] |
| I324 | 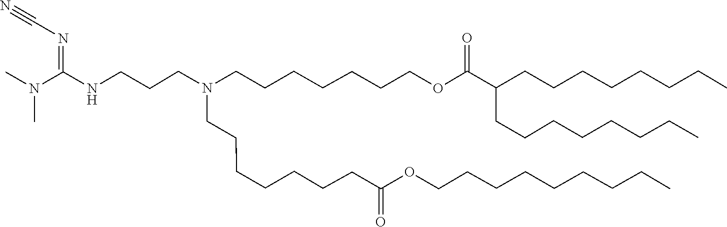 [SMILES: CCCCCCCCCOC(=O)CCCCCCCN(CCCCCCCOC(=O)C(CCCCCCCC)CCCCCCCC)CCCN/C(=N/C#N)N(C)C] |
| I325 | 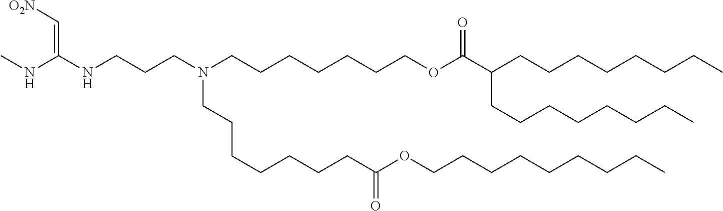 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I326 | 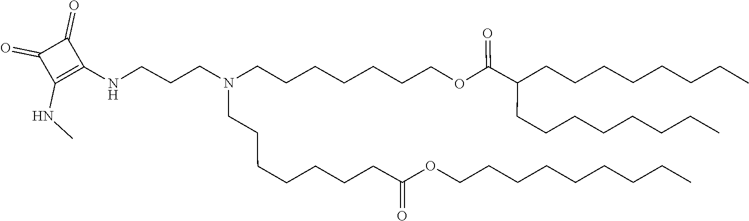 [SMILES: CCCCCCCCCOC(=O)CCCCCCCN(CCCCCCCOC(=O)C(CCCCCCCC)CCCCCCCC)CCCNc1c(NC)c(=O)c1=O] |
| I327 | 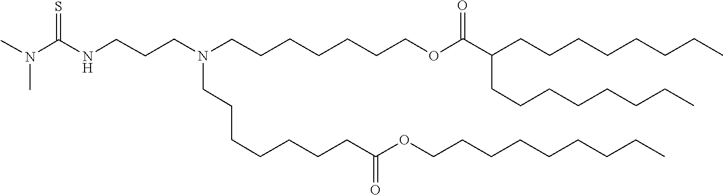 [SMILES: CCCCCCCCCOC(=O)CCCCCCCN(CCCCCCCOC(=O)C(CCCCCCCC)CCCCCCCC)CCCNC(=S)N(C)C] |
| I328 | 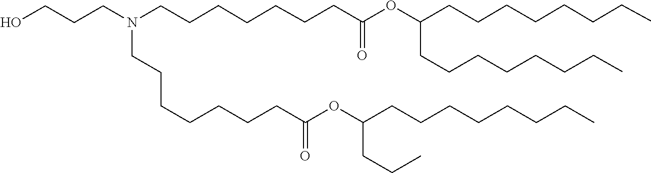 [SMILES: CCCCCCCCC(CCC)OC(=O)CCCCCCCN(CCCO)CCCCCCCC(=O)OC(CCCCCCCC)CCCCCCCC] |
| I329 | 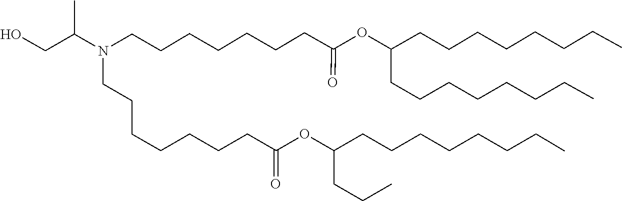 [SMILES: CCCCCCCCC(CCC)OC(=O)CCCCCCCN(CCCCCCCC(=O)OC(CCCCCCCC)CCCCCCCC)C(C)CO] |
| I330 | 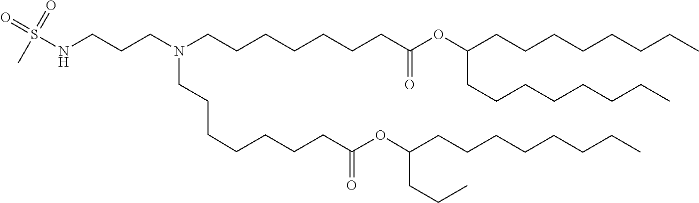 [SMILES: CCCCCCCCC(CCC)OC(=O)CCCCCCCN(CCCCCCCC(=O)OC(CCCCCCCC)CCCCCCCC)CCCNS(C)(=O)=O] |
| I331 | 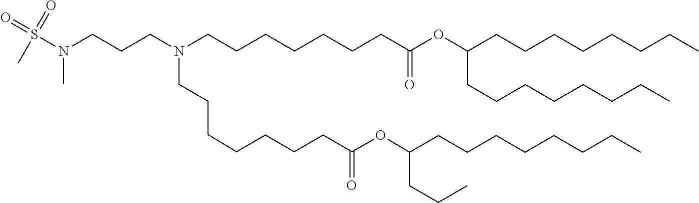 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I332 | 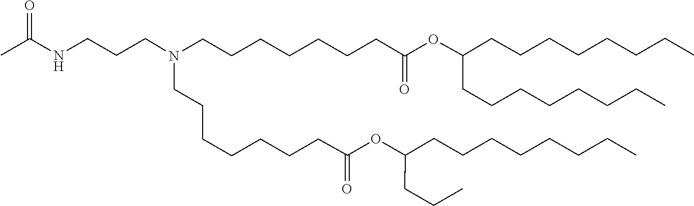 [SMILES: CCCCCCCCC(CCC)OC(=O)CCCCCCCN(CCCCCCCC(=O)OC(CCCCCCCC)CCCCCCCC)CCCNC(C)=O] |
| I333 | 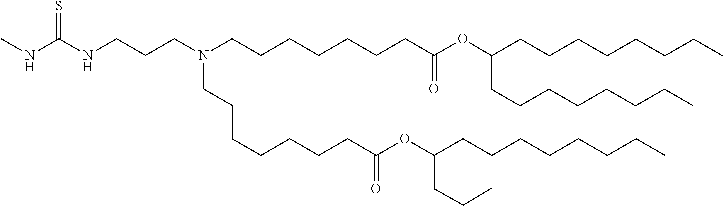 [SMILES: CCCCCCCCC(CCC)OC(=O)CCCCCCCN(CCCCCCCC(=O)OC(CCCCCCCC)CCCCCCCC)CCCNC(=S)NC] |
| I334 | 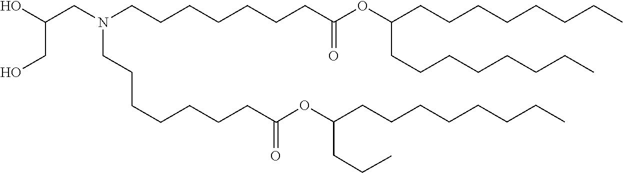 [SMILES: CCCCCCCCC(CCC)OC(=O)CCCCCCCN(CCCCCCCC(=O)OC(CCCCCCCC)CCCCCCCC)CC(O)CO] |
| I335 | 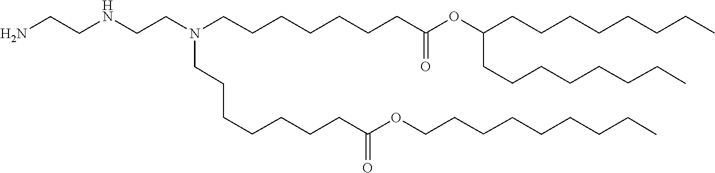 [SMILES: CCCCCCCCCOC(=O)CCCCCCCN(CCCCCCCC(=O)OC(CCCCCCCC)CCCCCCCC)CCNCCN] |
| I336 | 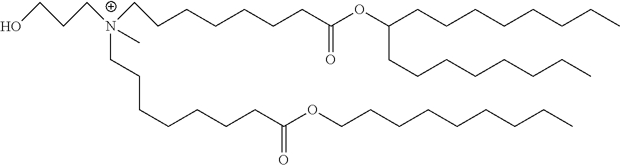 [SMILES: CCCCCCCCCOC(=O)CCCCCCC[N+](C)(CCCO)CCCCCCCC(=O)OC(CCCCCCCC)CCCCCCCC] |
| I337 | 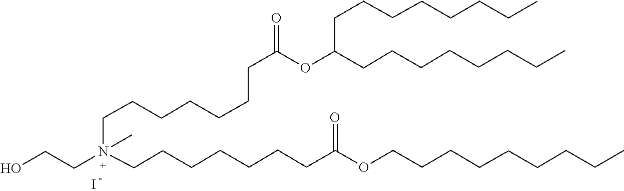 |
| I338 | 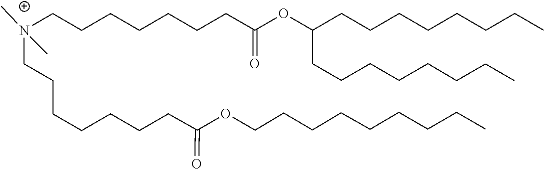 |
| I339 | 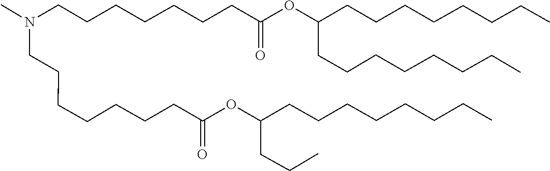 [SMILES: CCCCCCCCC(CCC)OC(=O)CCCCCCCN(C)CCCCCCCC(=O)OC(CCCCCCCC)CCCCCCCC] |
| I340 | 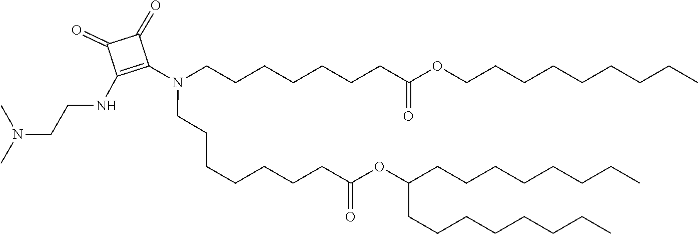 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I341 | 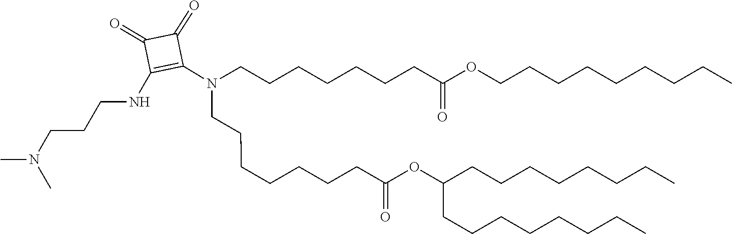 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I342 | 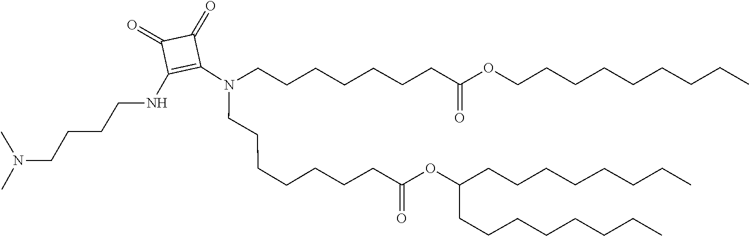 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I343 | 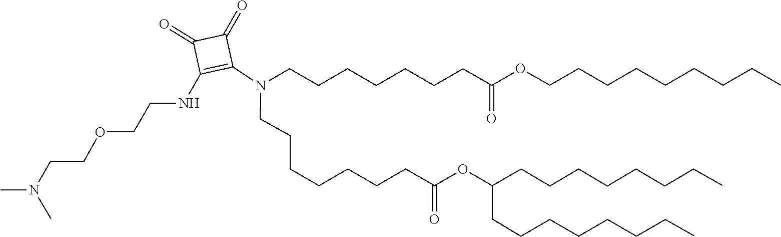 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I344 | 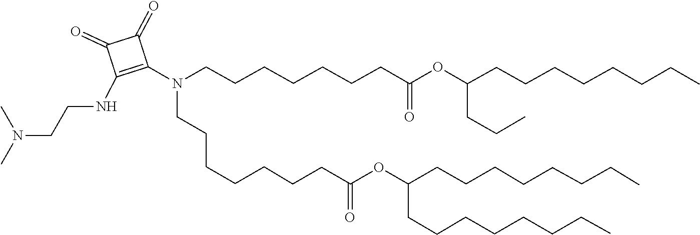 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I345 | 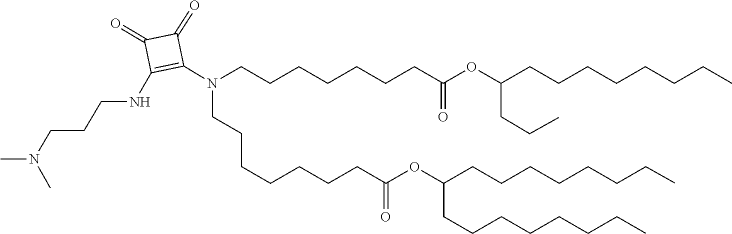 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I346 | 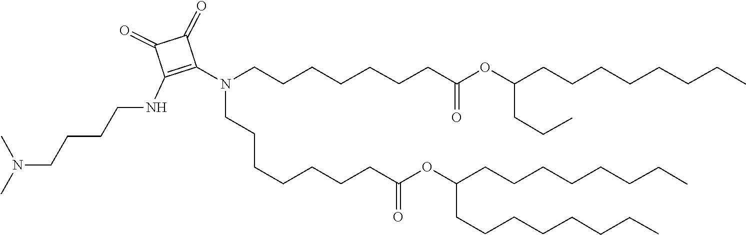 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I347 | 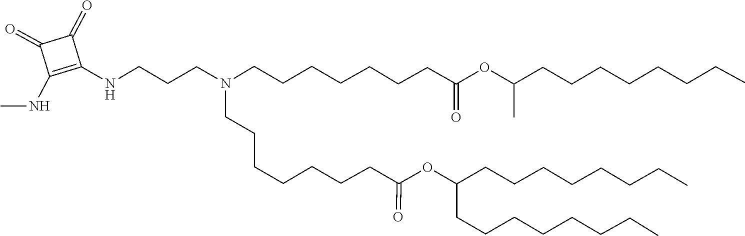 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I348 | 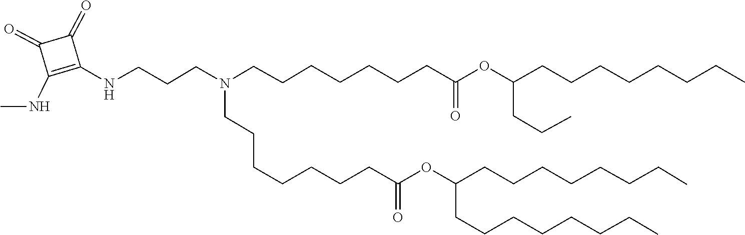 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I349 | 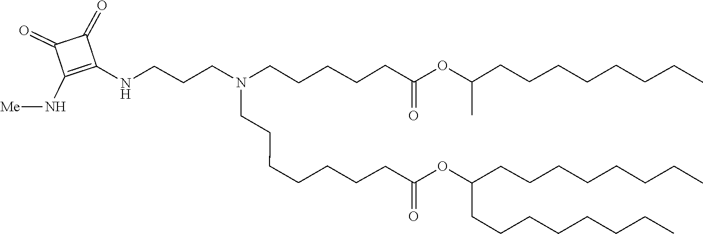 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I350 | 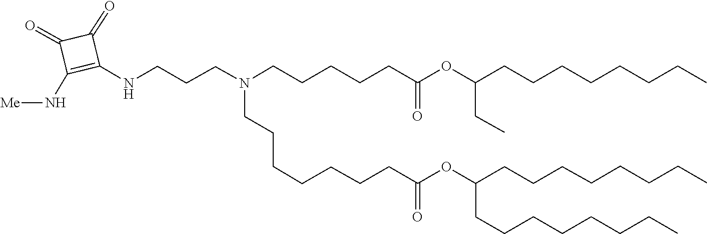 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I351 | 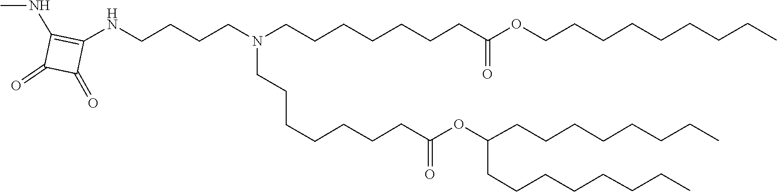 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I352 | 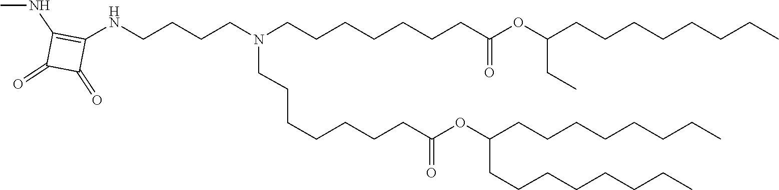 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I353 | 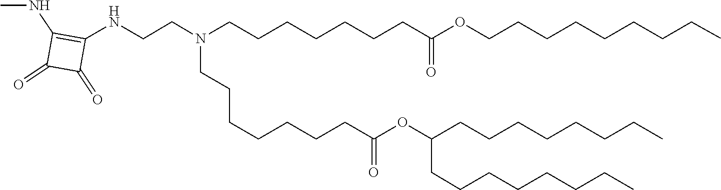 [SMILES: CCCCCCCCCOC(=O)CCCCCCCN(CCCCCCCC(=O)OC(CCCCCCCC)CCCCCCCC)CCNc1c(NC)c(=O)c1=O] |
| I354 | 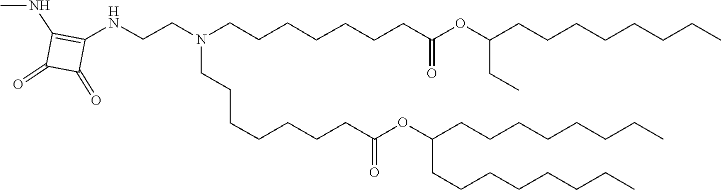 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I355 | 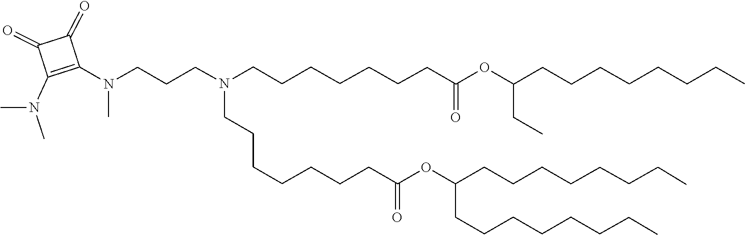 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |

In some embodiments, a lipid of the disclosure comprises Compound I-340A:

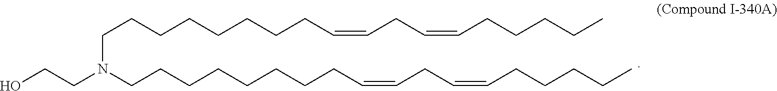 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I Ha), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

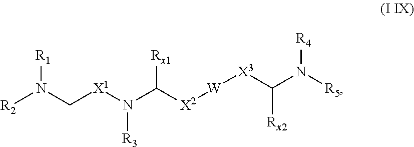

or salts or isomers thereof, wherein

### W is

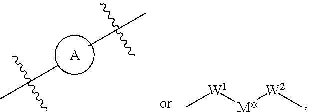

ring A is

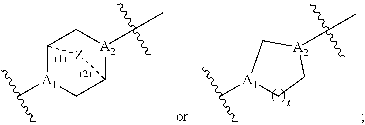

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting
of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(OXOR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R)—;
each R⁶ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

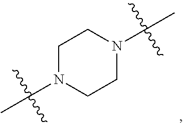 [SMILES: *N1CCN(*)CC1]

then
i) at least one of X¹, X², and X³ is not —CH₂—; and/or
ii) at least one of R¹, R², R³, R⁴, and R⁵ is —R″MR′.

In some embodiments, the compound is of any of formulae (I IXa1)-(I IXa8):

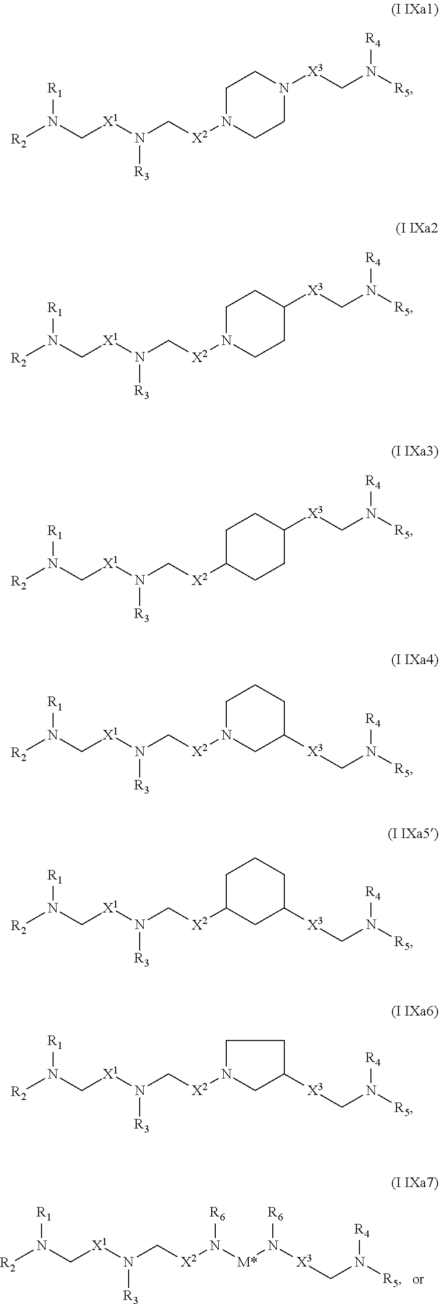

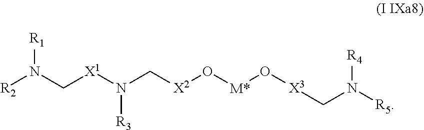

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

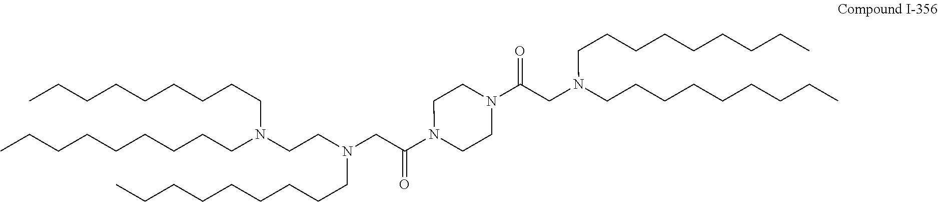 [SMILES: CCCCCCCCCN(CCCCCCCCC)CCN(CCCCCCCCC)CC(=O)N1CCN(C(=O)CN(CCCCCCCCC)CCCCCCCCC)CC1]

(also referred to herein as Compound M), or a salt thereof.

In some embodiments, the ionizable lipid is

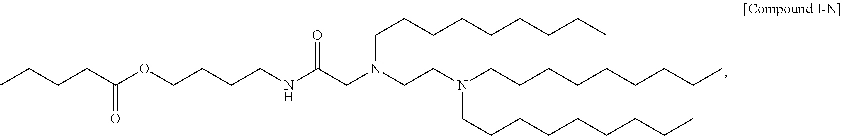 [SMILES: CCCCCCCCCN(CCCCCCCCC)CCN(CCCCCCCCC)CC(=O)NCCCCOC(=O)CCCC]

or a salt thereof.

In some embodiments, the ionizable lipid is

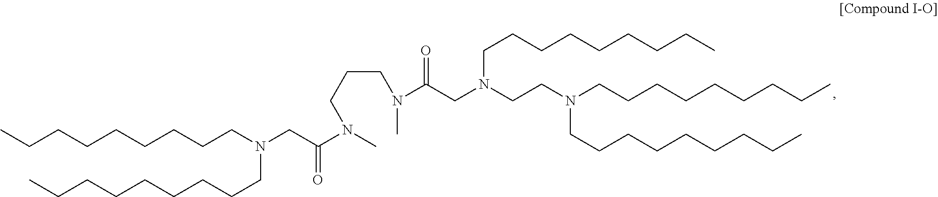 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.

In some embodiments, the ionizable lipid is

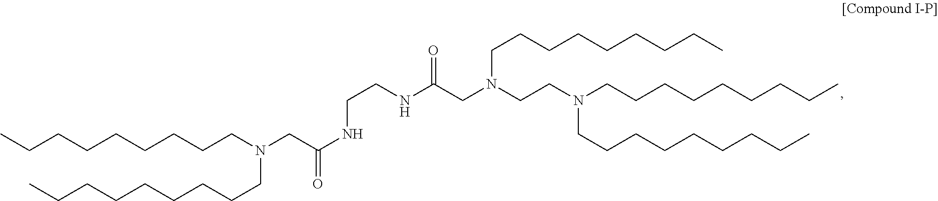 [SMILES: CCCCCCCCCN(CCCCCCCCC)CCN(CCCCCCCCC)CC(=O)NCCNC(=O)CN(CCCCCCCCC)CCCCCCCCC]

or a salt thereof.

In some embodiments, the ionizable lipid is

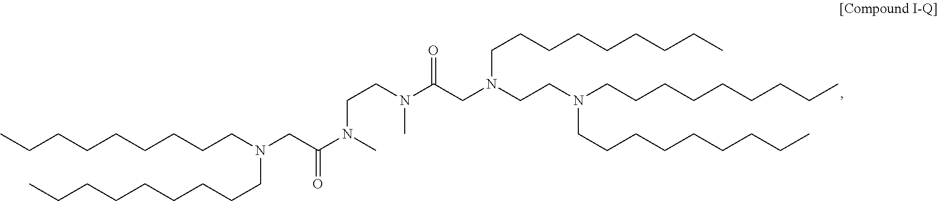 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (I IB), (II), (Ha), (IIb), (IIc), (IId), (He), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (II), (ha), (IIb), (IIc), (IId), (He), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8)) (each of these preceded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (Ha), (IIb), (IIc), (IId), (The), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (IA), (IB), (II), (Ha), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (ha), (IIb), (IIc), (IId), (He), (IIf), (Hg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 45 mol % in the lipid composition.

In one specific embodiment, the amount of the ionizable amino lipid of the invention, e.g. a compound having any of Formula (I), (IA), (IB), (II), (ha), (IIb), (IIc), (IId), (He), (IIf), (Hg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 40 mol % in the lipid composition.

In one specific embodiment, the amount of the ionizable amino lipid of the invention, e.g. a compound having any of Formula (I), (IA), (IB), (II), (ha), (IIb), (IIc), (IId), (He), (IIf), (Hg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 50 mol % in the lipid composition.

In addition to the ionizable amino lipid disclosed herein, e.g. a compound having any of Formula (I), (IA), (IB), (II), (ha), (IIb), (IIc), (IId), (He), (IIf), (Hg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3p)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety. For example, the ionizable amino lipids include, but not limited to:

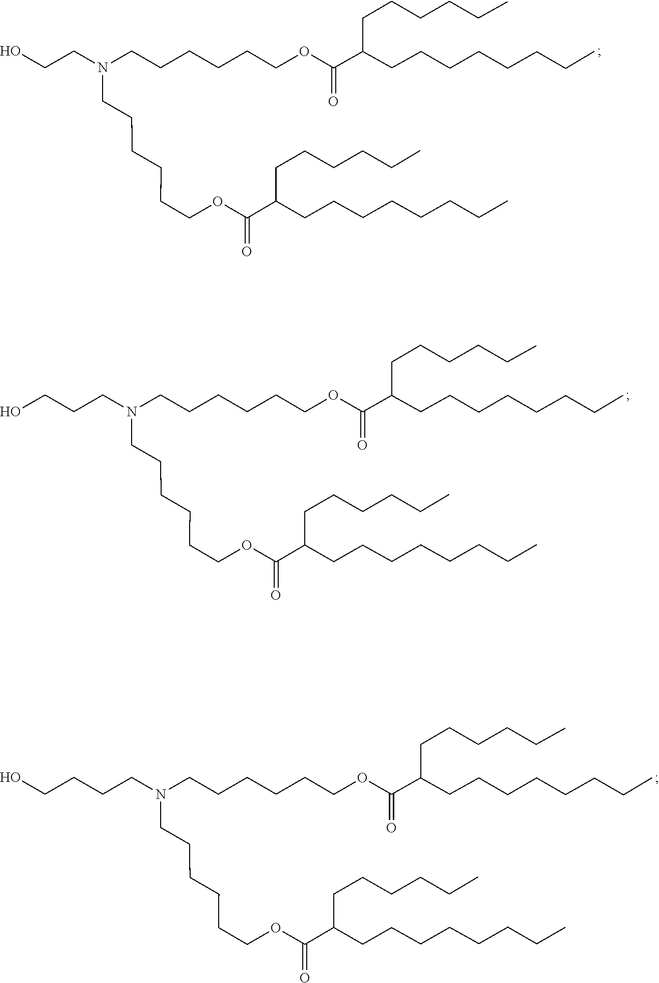 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids of the invention can also be the compounds disclosed in International Publication No. WO 2015/199952 A1, hereby incorporated by reference in its entirety. For example, the ionizable amino lipids include, but not limited to:

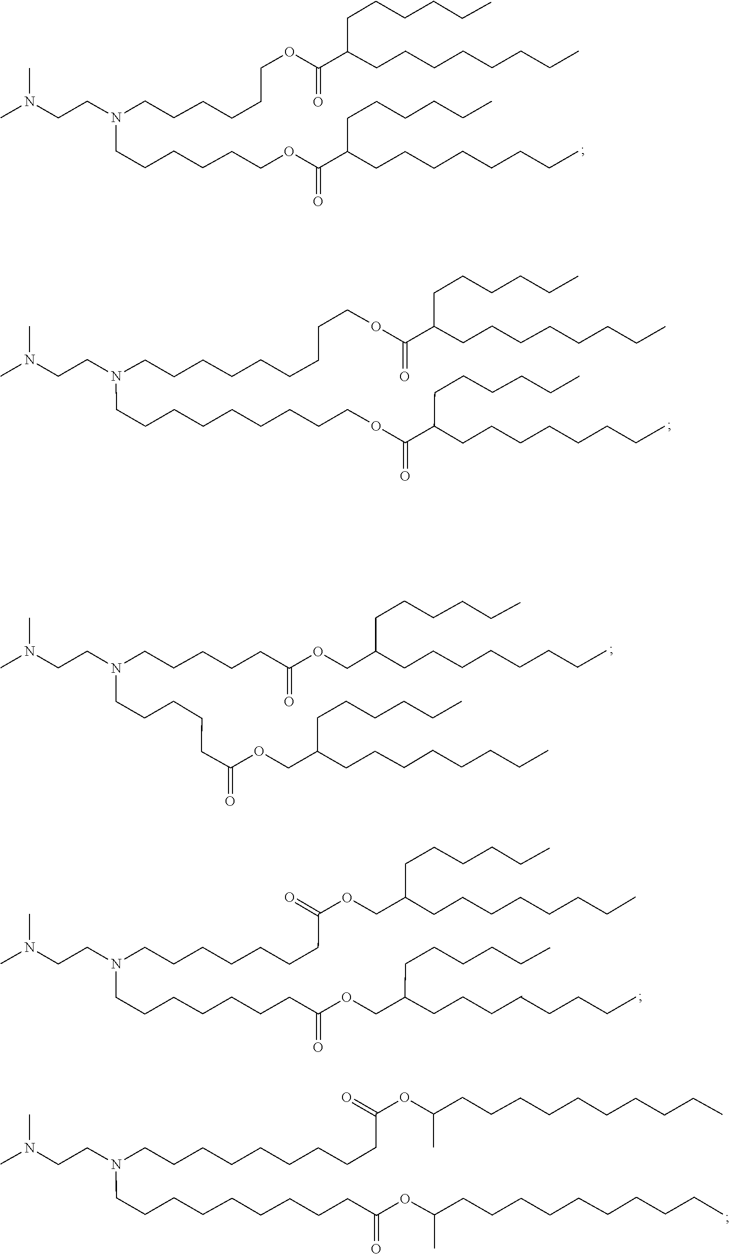

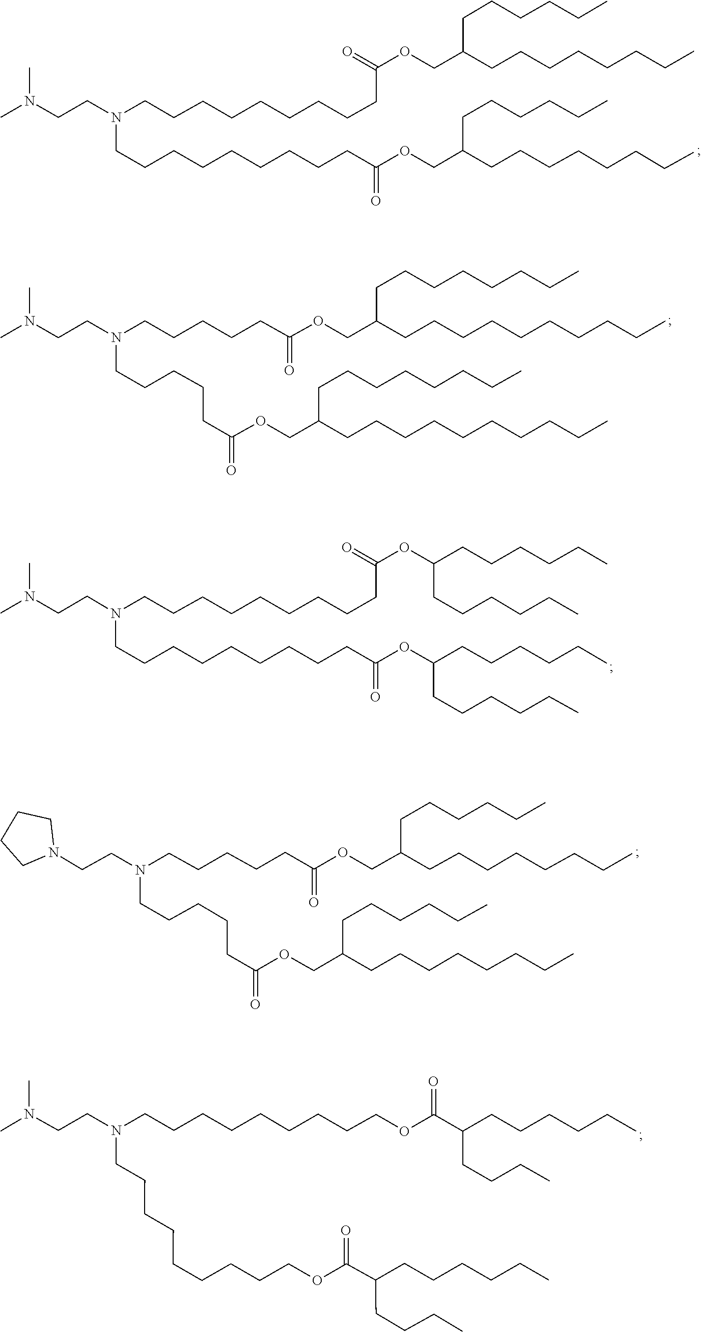

and any combination thereof.

In any of the foregoing or related aspects, the ionizable lipid of the LNP of the disclosure comprises a compound included in any e.g. a compound having any of Formula (I), (IA), (IB), (II), (Ha), (IIb), (IIc), (IId), (He), (IIf), (Hg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity).

In any of the foregoing or related aspects, the ionizable lipid of the LNP of the disclosure comprises a compound comprising any of Compound Nos. 11-356.

In any of the foregoing or related aspects, the ionizable lipid of the LNP of the disclosure comprises at least one compound selected from the group consisting of: Compound Nos. I 18, I 25, I 48, I 50, I 109, I 111, I 113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18, I 25, I 48, I 50, I 109, I 111, I 181, I 182, I 292, I 301, I 321, I 326, I 328, and I 330. In another embodiment, the ionizable lipid of the LNP of the disclosure comprises Compound 18. In another embodiment, the ionizable lipid of the LNP of the disclosure comprises Compound 25.

In any of the foregoing or related aspects, the synthesis of compounds of the invention, e.g., compounds comprising any of Compound Nos. 1-356, follows the synthetic descriptions in U.S. Provisional Patent Application No. 62/733,315, filed Sep. 19, 2018.

### Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

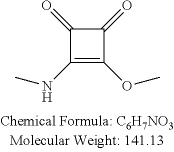

To a solution of 3,4-dimethoxy-3-cyclobutene-1,2-dione (1 g, 7 mmol) in 100 mL diethyl ether was added a 2 M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

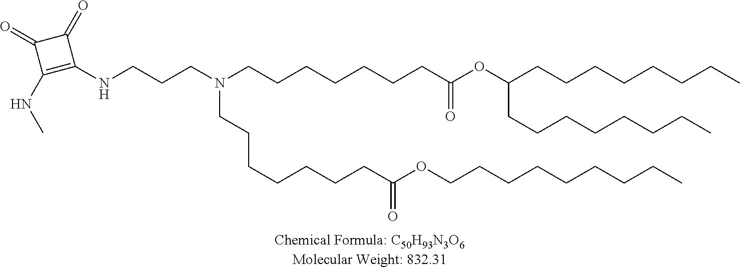 [SMILES: CCCCCCCCCOC(=O)CCCCCCCN(CCCCCCCC(=O)OC(CCCCCCCC)CCCCCCCC)CCCNc1c(NC)c(=O)c1=O]

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH4OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

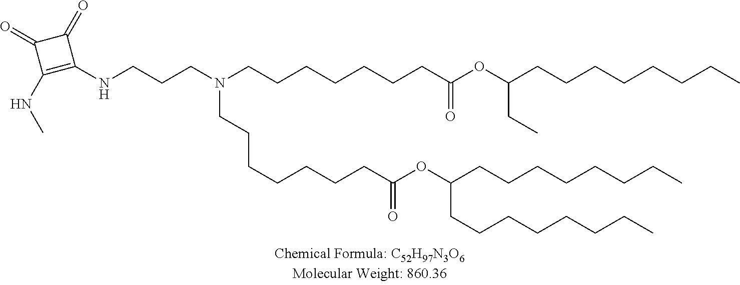 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH40H, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

### Cholesterol/Structural Lipids

The LNP described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

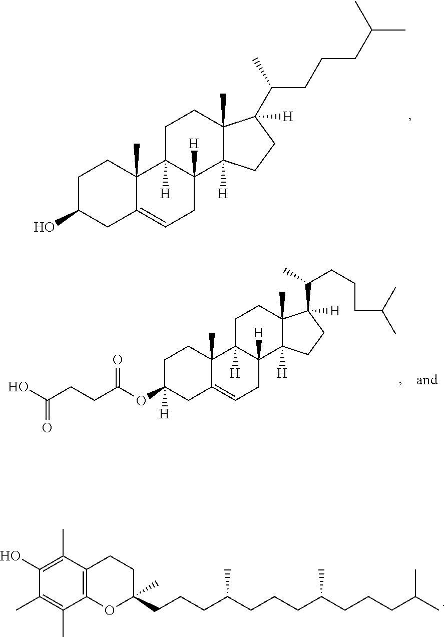

The target cell target cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

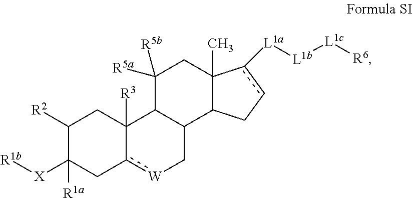

where
R^{1a} is H, optionally substituted C₁-C₆ alkyl, optionally substituted C₂-C₆ alkenyl, or optionally substituted C₂-C₆ alkynyl;
X is O or S;
R^{1b} is H, optionally substituted C₁-C₆ alkyl, or

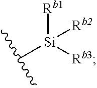

each of R^{b1}, R^{b2}, and R^{b3} is, independently, optionally substituted C₁-C₆ alkyl or optionally substituted C₆-C₁₀ aryl;
R² is H or OR^{A}, where R^{A} is H or optionally substituted C₁-C₆ alkyl;
R³ is H or

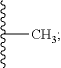

each [symbol] independently represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

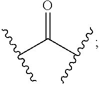 [SMILES: *C(*)=O]

L^{1a} is absent,

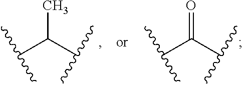

L^{1b} is absent,

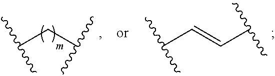

m is 1, 2, or 3;
L^{1c} is absent,

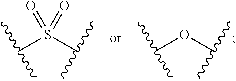

and
R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl,
   or a pharmaceutically acceptable salt thereof.

In some embodiments, the compound has the structure of Formula SIa:

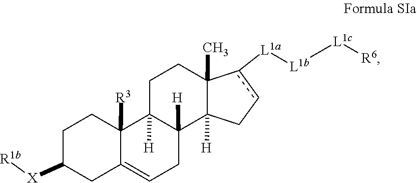

or a pharmaceutically acceptable salt thereof.

In some embodiments, the compound has the structure of Formula SIb:

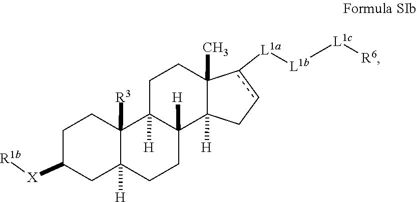

or a pharmaceutically acceptable salt thereof.

In some embodiments, the compound has the structure of Formula SIc:

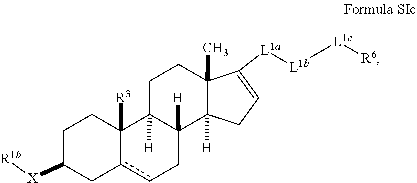

or a pharmaceutically acceptable salt thereof.

In some embodiments, the compound has the structure of Formula SId:

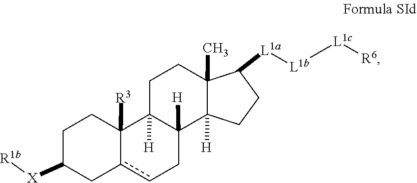

or a pharmaceutically acceptable salt thereof.

In some embodiments, L^{1a} is absent. In some embodiments, L^{1a} is

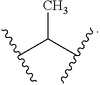 [SMILES: *C(*)C]

In some embodiments, L^{1a} is

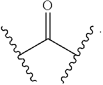 [SMILES: *C(*)=O]

In some embodiments, L^{1b} is absent. In some embodiments, L^{1b} is

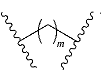

In some embodiments, L^{1b} is

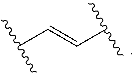 [SMILES: */C=C/*]

In some embodiments, m is 1 or 2. In some embodiments, m is 1. In some embodiments, m is 2.

In some embodiments, L^{1c} is absent. In some embodiments, L^{1c} is

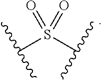 [SMILES: *S(*)(=O)=O]

In some embodiments, L^{1c} is

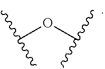 [SMILES: *O*]

In some embodiments, R⁶ is optionally substituted C₆-C₁₀ aryl.

In some embodiments, R⁶ is

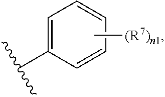

where

n1 is 0, 1, 2, 3, 4, or 5; and

each R⁷ is, independently, halo or optionally substituted C₁-C₆ alkyl.

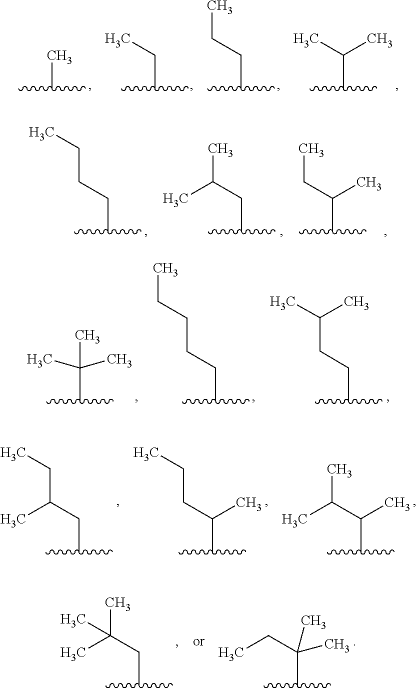

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

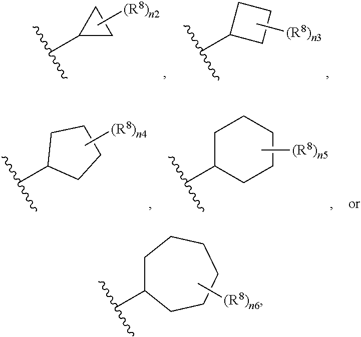

where
n2 is 0, 1, 2, 3, 4, or 5;
n3 is 0, 1, 2, 3, 4, 5, 6, or 7;
n4 is 0, 1, 2, 3, 4, 5, 6, 7, 8, or 9;
n5 is 0, 1, 2, 3, 4, 5, 6, 7, 8, 9, 10, or 11;
n6 is 0, 1, 2, 3, 4, 5, 6, 7, 8, 9, 10, 11, 12, or 13; and
each R⁸ is, independently, halo or optionally substituted C₁-C₆ alkyl.

In some embodiments, each R⁸ is, independently,

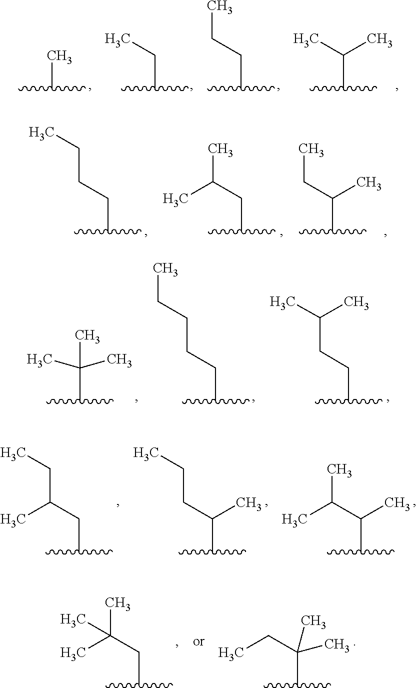

In some embodiments, R⁶ is optionally substituted C₃-C₁₀ polycycloalkyl.

In some embodiments. R⁶ is

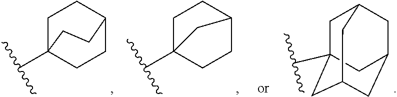

In some embodiments, R⁶ is optionally substituted C₃-C₁₀ cycloalkenyl.

In some embodiments, R⁶ is

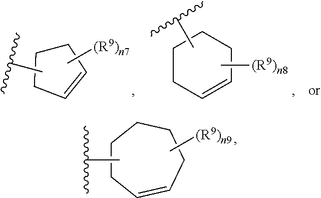

where
n7 is 0, 1, 2, 3, 4, 5, 6, or 7;
n8 is 0, 1, 2, 3, 4, 5, 6, 7, 8, or 9;
n9 is 0, 1, 2, 3, 4, 5, 6, 7, 8, 9, 10, or 11; and
each R⁹ is, independently, halo or optionally substituted C₁-C₆ alkyl.

In some embodiments, R⁶ is

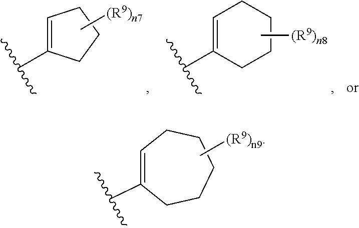

In some embodiments, each R is, independently,

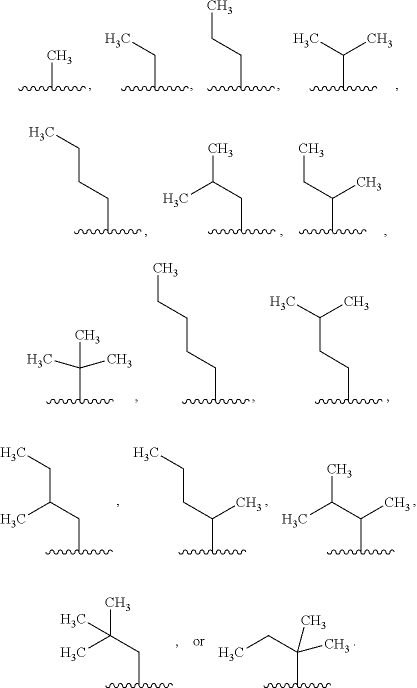

In some embodiments, R⁶ is optionally substituted C₂-C₉ heterocyclyl.

In some embodiments, R⁶ is

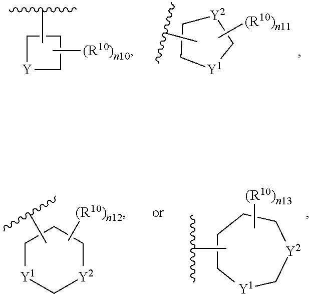

where
n10 is 0, 1, 2, 3, 4, or 5;
n11 is 0, 1, 2, 3, 4, or 5;
n12 is 0, 1, 2, 3, 4, 5, 6, or 7;
n13 is 0, 1, 2, 3, 4, 5, 6, 7, 8, or 9;
each R¹⁰ is, independently, halo or optionally substituted C₁-C₆ alkyl; and
each of Y¹ and Y² is, independently, O, S, NR^{B}, or CR^{11a}R^{11b},
where R^{B} is H or optionally substituted C₁-C₆ alkyl;
each of R^{11a} and R^{11b} is, independently, H, halo, or optionally substituted C₁-C₆ alkyl; and
if Y² is CR^{11a}R^{11b}, then Y′ is O, S, or NR^{B}.

In some embodiments, Y¹ is O.

In some embodiments, Y² is O. In some embodiments, Y² is CR^{11a}R^{11b}.

In some embodiments, each R¹⁰ is, independently,

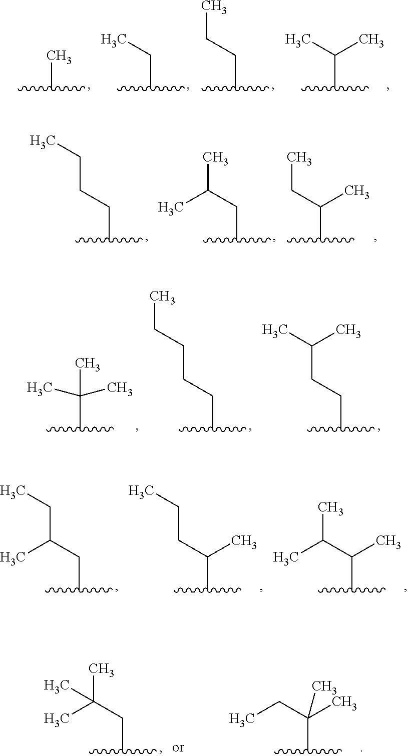

In some embodiments, R⁶ is optionally substituted C₂-C₉ heteroaryl.

In some embodiments, R⁶ is

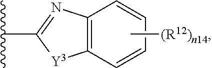

where
Y³ is NR^{C}, O, or S
n14 is 0, 1, 2, 3, or 4;
R^{C} is H or optionally substituted C₁-C₆ alkyl; and
each R¹² is, independently, halo or optionally substituted C₁-C₆ alkyl.

In some embodiments, R⁶ is

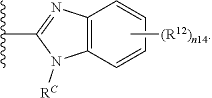

In some embodiments, R⁶ is

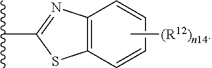

In an aspect, the structural lipid of the invention features a compound having the structure of Formula SII:

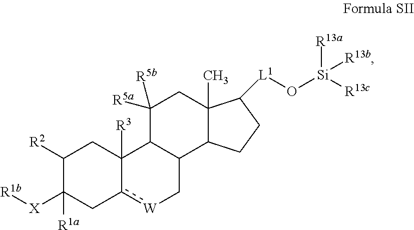

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

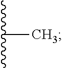

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

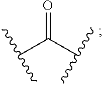 [SMILES: *C(*)=O]

L¹ is optionally substituted C₁-C₆ alkylene; and
each of R^{13a}, R^{13b}, and R^{13c} is, independently, optionally substituted C₁-C₆ alkyl or optionally substituted C₆-C₁₀ aryl,
   or a pharmaceutically acceptable salt thereof.

In some embodiments, the compound has the structure of Formula SIIa:

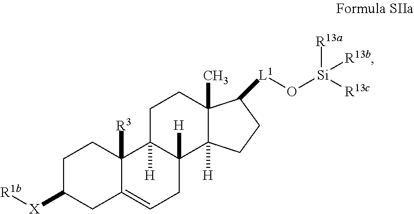

or a pharmaceutically acceptable salt thereof.

In some embodiments, the compound has the structure of Formula SIIb:

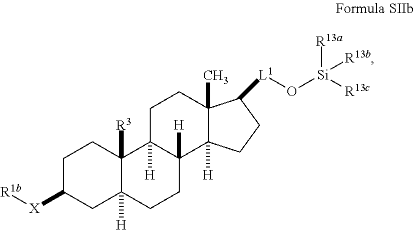

or a pharmaceutically acceptable salt thereof.

In some embodiments, L¹ is

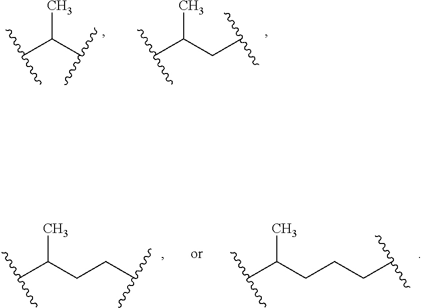

In some embodiments, each of R^{13a}, R^{13b}, and R^{13c} is, independently,

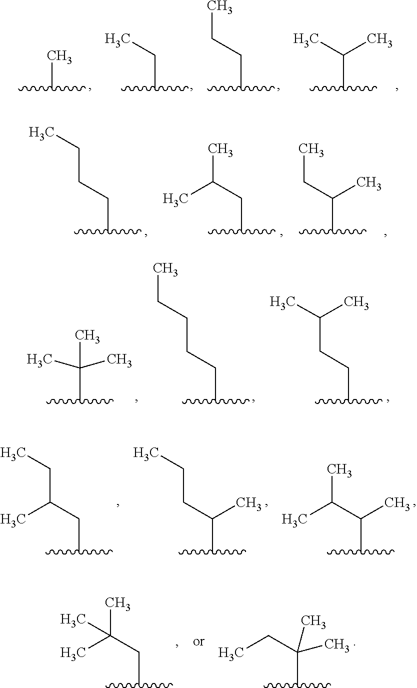

In an aspect, the structural lipid of the invention features a compound having the structure of Formula SII:

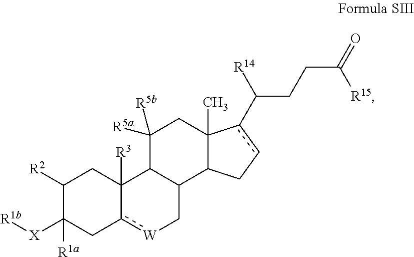

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

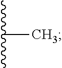

each [symbol] independently represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;
each of R^{5a} and R^{5b} is, independently, H or OR^{A}, or R^{5a} and R^{5b}, together with the atom to which each is attached, combine to form

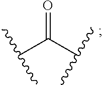 [SMILES: *C(*)=O]

R¹⁴ is H or C₁-C₆ alkyl; and
R¹⁵ is

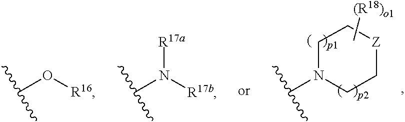

where
R¹⁶ is H or optionally substituted C₁-C₆ alkyl;
   R¹⁷ is H, OR^{17c}, optionally substituted C₆-C₁₀ aryl, or optionally substituted C₁-C₆ alkyl;
R^{17c} is H or optionally substituted C₁-C₆ alkyl;
o1 is 0, 1, 2, 3, 4, 5, 6, 7, or 8;
p1 is 0, 1, or 2;
p2 is 0, 1, or 2;
Z is CH₂O, S, or NR^{D}, where R^{D} is H or optionally substituted C₁-C₆ alkyl; and
each R¹⁸ is, independently, halo or optionally substituted C₁-C₆ alkyl,
   or a pharmaceutically acceptable salt thereof.

In some embodiments, the compound has the structure of Formula SIIIa:

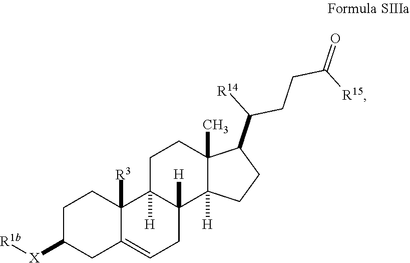

or a pharmaceutically acceptable salt thereof.

In some embodiments, the compound has the structure of Formula SIIIb:

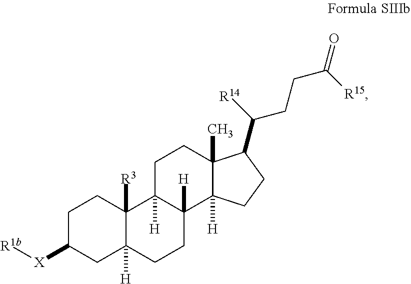

or a pharmaceutically acceptable salt thereof.

In some embodiments, R¹⁴ is H,

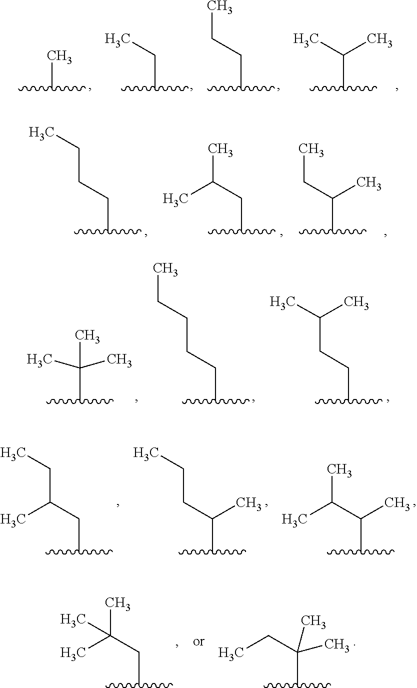

In some embodiments, R¹⁴ is

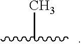

In some embodiments, R¹⁵ is

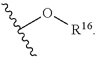

In some embodiments, R⁵ is

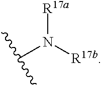

In some embodiments, R¹⁶ is H. In some embodiments, R¹⁶ is

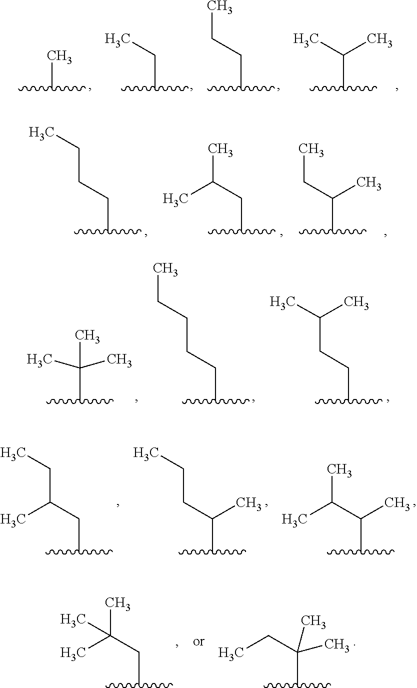

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

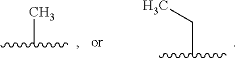

In some embodiments, R^{17c} is H. In some embodiments, R^{17c} is A

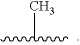

In some embodiments, R¹⁵ is

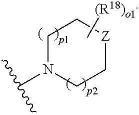

In some embodiments, each R¹⁸ is, independently,

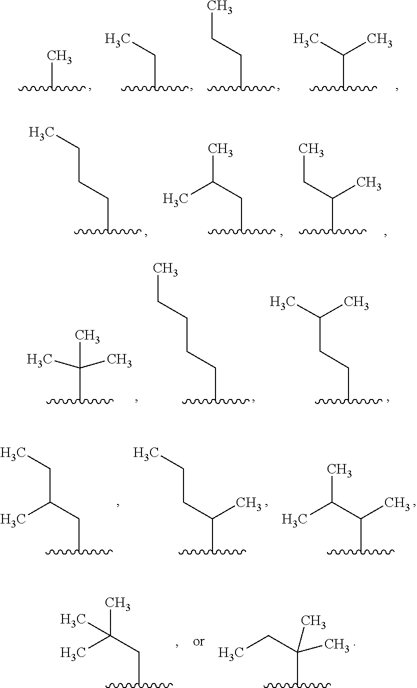

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

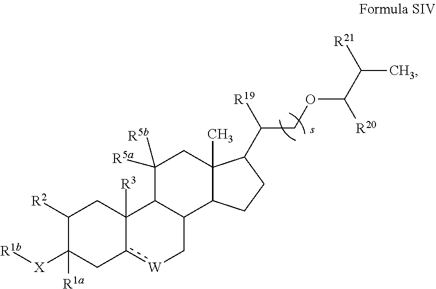

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

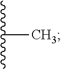

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

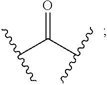 [SMILES: *C(*)=O]

s is 0 or 1;
R¹⁹ is H or C₁-C₆ alkyl;
R²⁰ is C₁-C₆ alkyl;
R²¹ is H or C₁-C₆ alkyl,
   or a pharmaceutically acceptable salt thereof.

In some embodiments, the compound has the structure of Formula SIVa:

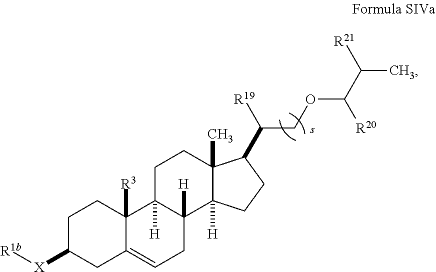

or a pharmaceutically acceptable salt thereof.

In some embodiments, the compound has the structure of Formula SIVb:

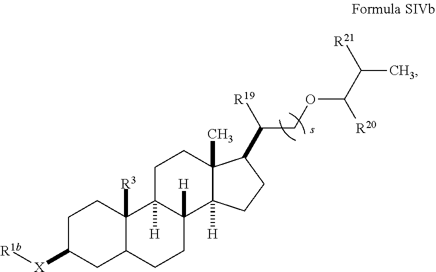

or a pharmaceutically acceptable salt thereof.

In some embodiments, R¹⁹ is H,

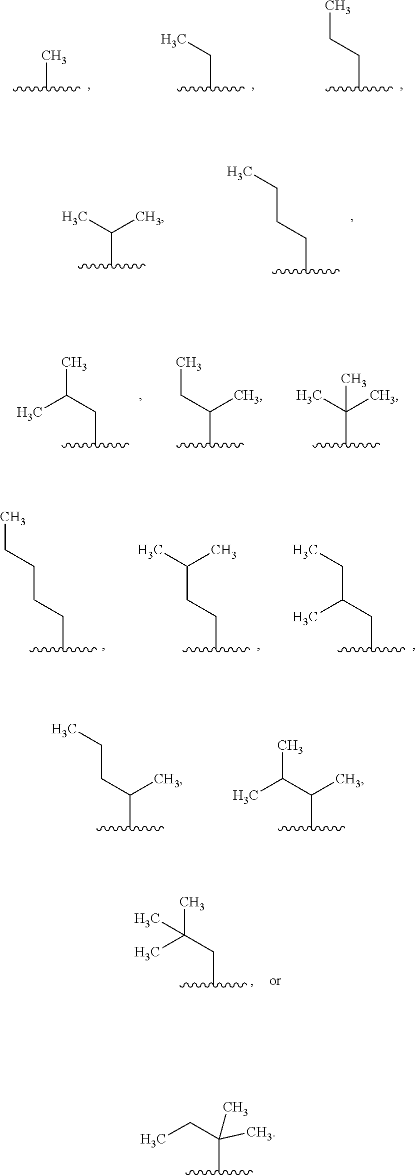

In some embodiments, R¹⁹ is

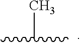

In some embodiments, R²⁰ is

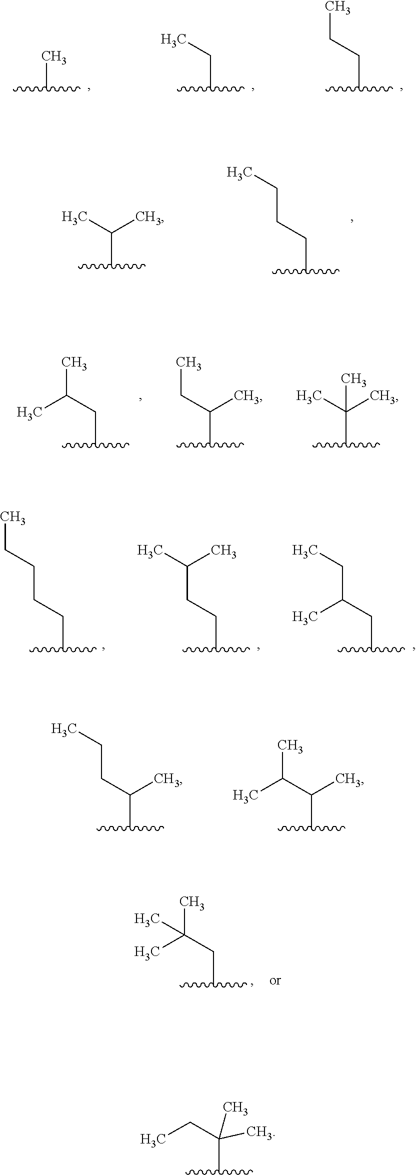

In some embodiments, R²¹ is H,

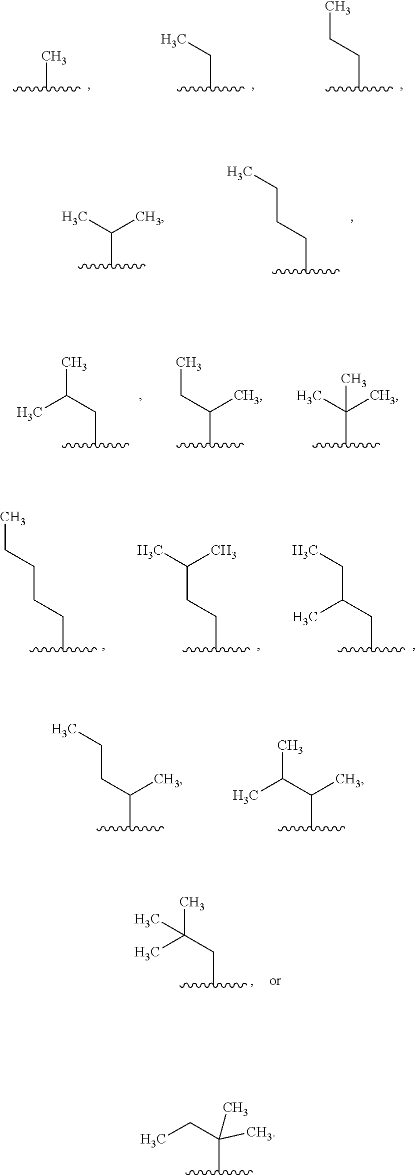

In an aspect, the structural lipid of the invention features, a compound having the structure of Formula SV:

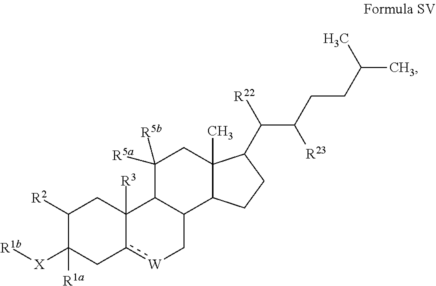

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

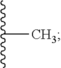

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form;

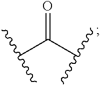 [SMILES: *C(*)=O]

R²² is H or C₁-C₆ alkyl; and
R²³ is halo, hydroxyl, optionally substituted C₁-C₆ alkyl, or optionally substituted C₁-C₆ heteroalkyl,
   or a pharmaceutically acceptable salt thereof.

In some embodiments, the compound has the structure of Formula SVa:

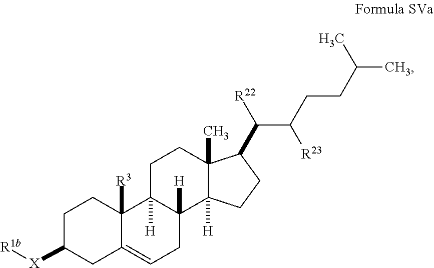

or a pharmaceutically acceptable salt thereof.

In some embodiments, the compound has the structure of Formula SVb:

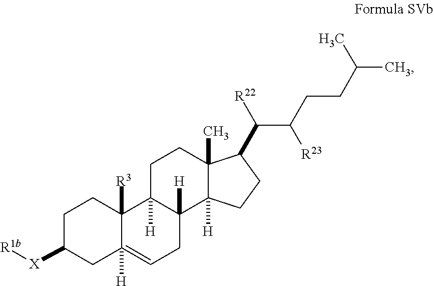

or a pharmaceutically acceptable salt thereof.

In some embodiments, R²² is H,

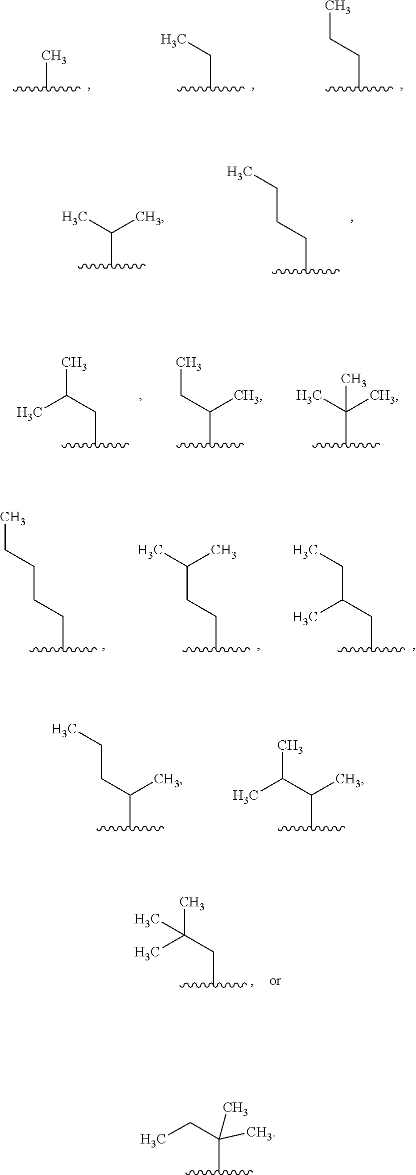

In some embodiments, R²² is

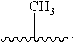

In some embodiments, R²³ is

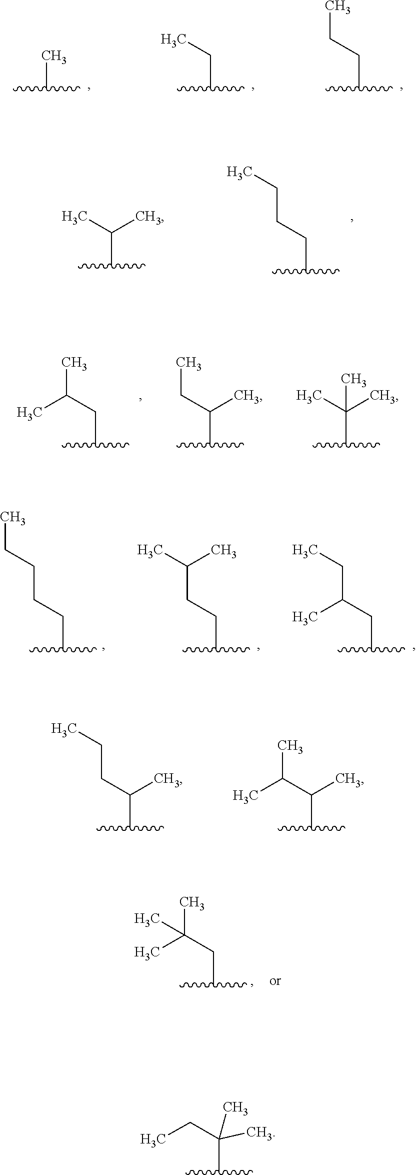

In an aspect, the structural lipid of the invention features a compound having the structure of Formula SVI:

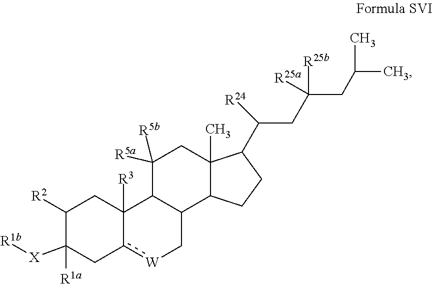

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

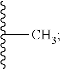

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b}.
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

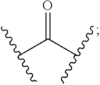 [SMILES: *C(*)=O]

R²⁴ is H or C₁-C₆ alkyl; and
each of R^{25a} and R^{25b} is C₁-C₆ alkyl,
   or a pharmaceutically acceptable salt thereof.

In some embodiments, the compound has the structure of Formula SVIa:

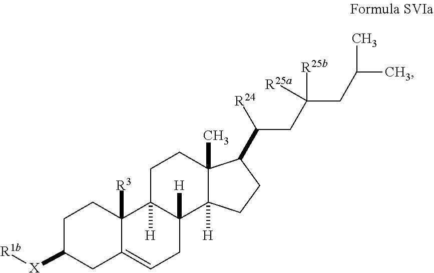

or a pharmaceutically acceptable salt thereof.

In some embodiments, the compound has the structure of Formula SVIb:

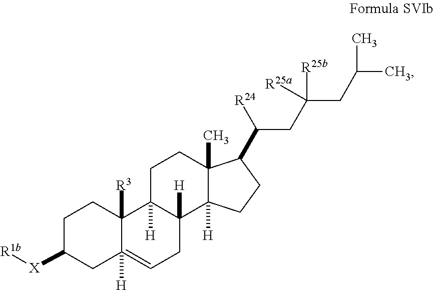

or a pharmaceutically acceptable salt thereof.

In some embodiments, R²⁴ is H,

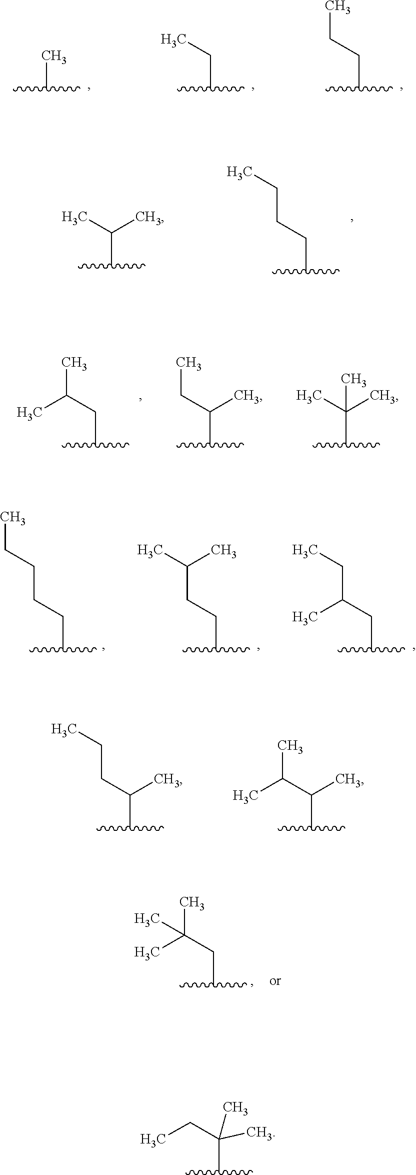

In some embodiments, R²⁴ is

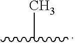

In some embodiments, each of R^{25a} and R^{25b} is, independently,

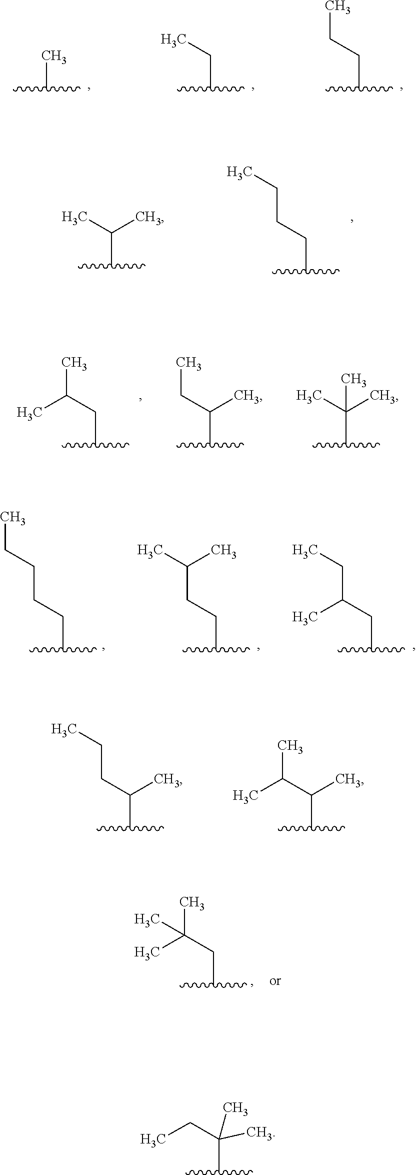

In an aspect, the structural lipid of the invention features a compound having the structure of Formula SVII:

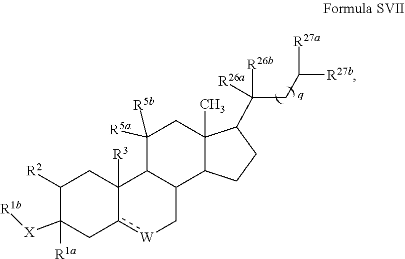

where
R^{1a} is H, optionally substituted C₁-C₆ alkyl, optionally substituted C₂-C₆ alkenyl, optionally substituted C₂-C₆ alkynyl, or

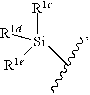

where each of R^{1c}, R^{1d}, and R^{1e} is, independently, optionally substituted C₁-C₆ alkyl or optionally substituted C₆-C₁₀ aryl;
X is O or S;
R^{1b} is H or optionally substituted C₁-C₆ alkyl;
R² is H or OR^{A}, where R^{A} is H or optionally substituted C₁-C₆ alkyl;
R³ is H or

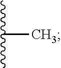

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

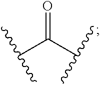 [SMILES: *C(*)=O]

q is 0 or 1;
each of R^{26a} and R^{26b} is, independently, H or optionally substituted C₁-C₆ alkyl, or R²⁶ and R^{26b}, together with the atom to which each is attached, combine to form

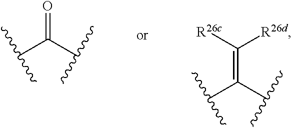

where each of R^{26c} and R²⁶ is, independently, H or optionally substituted C₁-C₆ alkyl; and
each of R^{27a} and R^{27b} is H, hydroxyl, or optionally substituted C₁-C₆ alkyl,
   or a pharmaceutically acceptable salt thereof.

In some embodiments, the compound has the structure of Formula SVIIa:

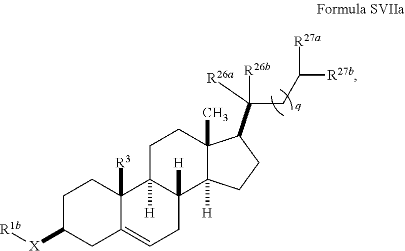

or a pharmaceutically acceptable salt thereof.

In some embodiments, the compound has the structure of Formula SVIIb:

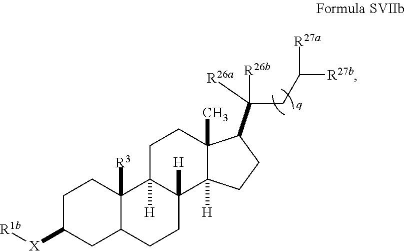

or a pharmaceutically acceptable salt thereof.

In some embodiments, R^{26a} and R^{26b} is, independently, H

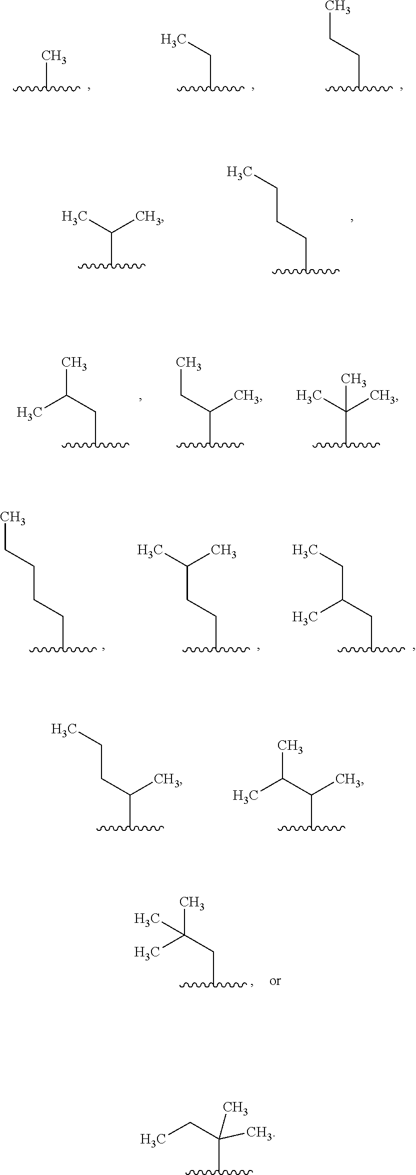

In some embodiments, R^{26a} and R^{26b}, together with the atom to which each is attached, combine to form

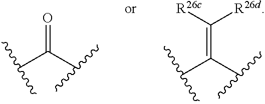

In some embodiments, R^{26a} and R^{26b}, together with the atom to which each is attached, combine to form

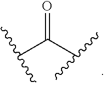 [SMILES: *C(*)=O]

In some embodiments, R^{26a} and R^{26b}, together with the atom to which each is attached, combine to form

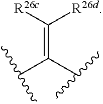

In some embodiments, where each of R^{26c} and R²⁶ is, independently, H,

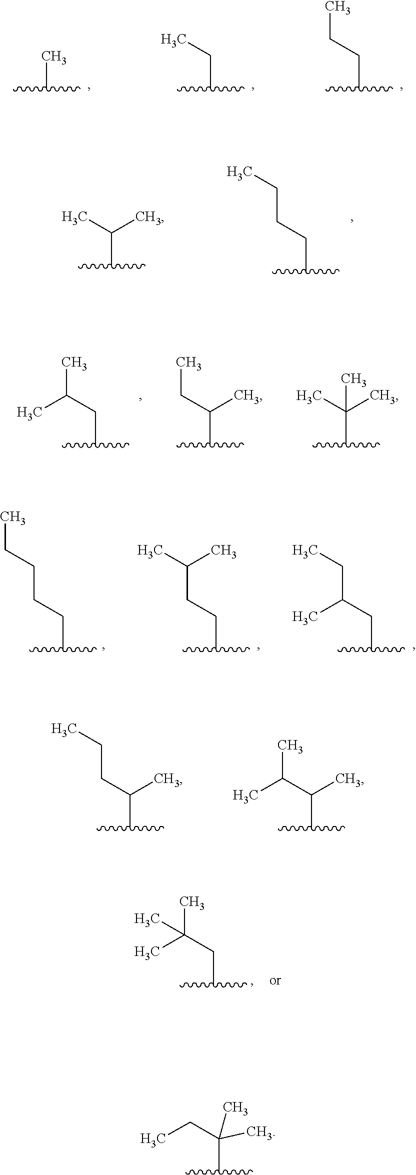

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

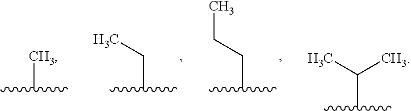

In an aspect, the structural lipid of the invention features a compound having the structure of Formula SVIII:

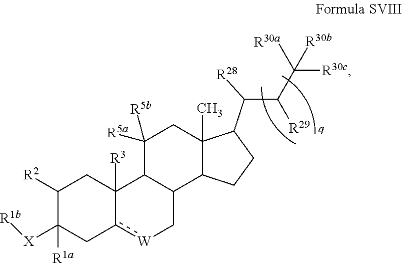

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

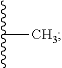

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b}.
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

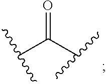 [SMILES: *C(*)=O]

R²⁸ is H or optionally substituted C₁-C₆ alkyl;
r is 1, 2, or 3;
each R²⁹ is, independently, H or optionally substituted C₁-C₆ alkyl; and
each of R^{30a}, R^{30b}, and R^{30c} is C₁-C₆ alkyl,
   or a pharmaceutically acceptable salt thereof.

In some embodiments, the compound has the structure of Formula SVIIIa:

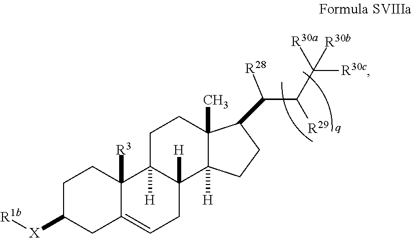

or a pharmaceutically acceptable salt thereof.

In some embodiments, the compound has the structure of Formula SVIIIb:

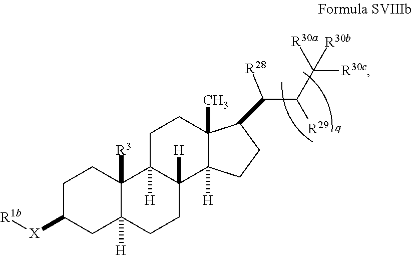

or a pharmaceutically acceptable salt thereof.

In some embodiments, R²⁸ is H,

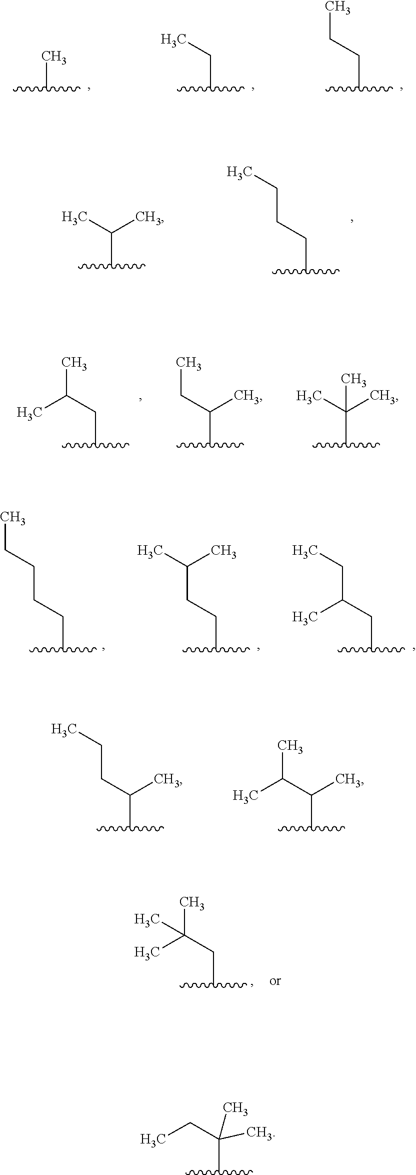

In some embodiments, R²⁸ is

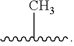

In some embodiments, each of R^{30a}, R^{30b}, and R^{30c} is, independently,

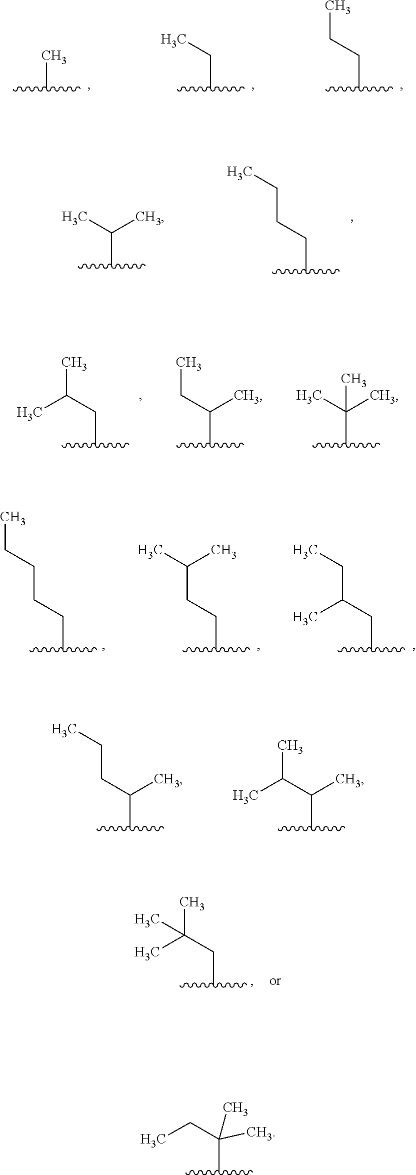

In some embodiments, r is 1. In some embodiments, r is 2. In some embodiments, r is 3.

In some embodiments, each R²⁹ is, independently, H

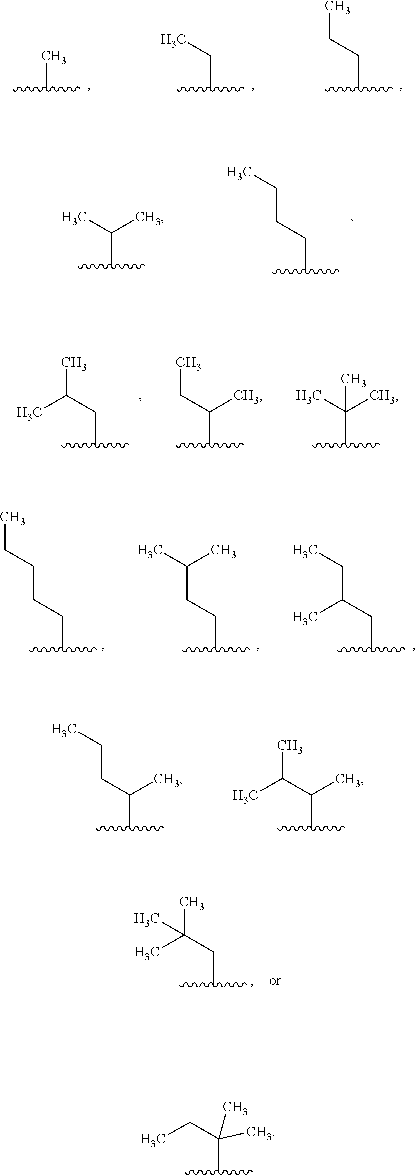

In some embodiments, each R²⁹ is, independently, H or

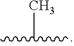

In an aspect, the structural lipid of the invention features a compound having the structure of Formula SIX:

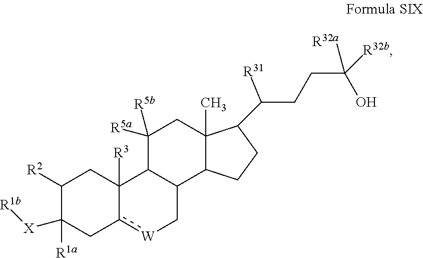

where
R^{1a} is H, optionally substituted C₁-C₆ alkyl, optionally substituted C₂-C₆ alkenyl, or optionally substituted C₂-C₆ alkynyl;
X is O or S;
R^{1b} is H or optionally substituted C₁-C₆ alkyl;
R² is H or OR^{A}, where R^{A} is H or optionally substituted C₁-C₆ alkyl;
R³ is H or

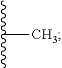

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

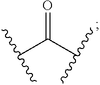 [SMILES: *C(*)=O]

R³¹ is H or C₁-C₆ alkyl; and
each of R^{32a} and R^{32b} is C₁-C₆ alkyl,
   or a pharmaceutically acceptable salt thereof.

In some embodiments, the compound has the structure of Formula SIXa:

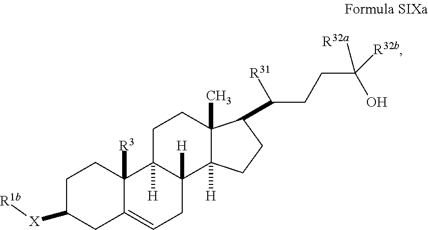

or a pharmaceutically acceptable salt thereof.

In some embodiments, the compound has the structure of Formula SIXb:

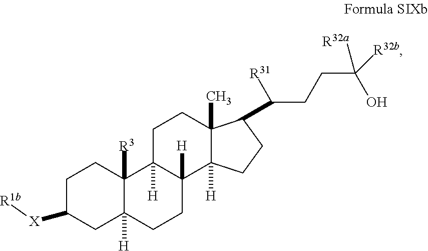

or a pharmaceutically acceptable salt thereof.

In some embodiments, R³¹ is H,

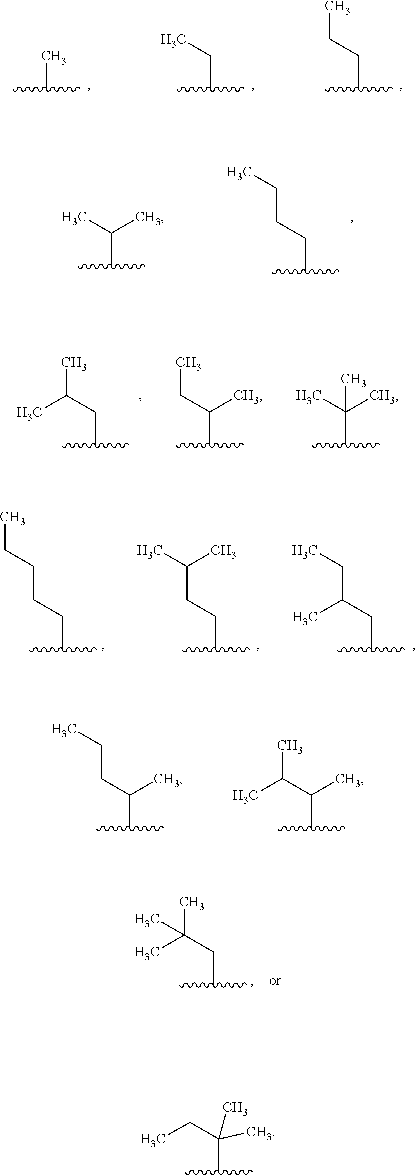

In some embodiments, R³¹ is

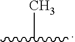

In some embodiments, each of R^{32a} and R^{32b} is, independently,

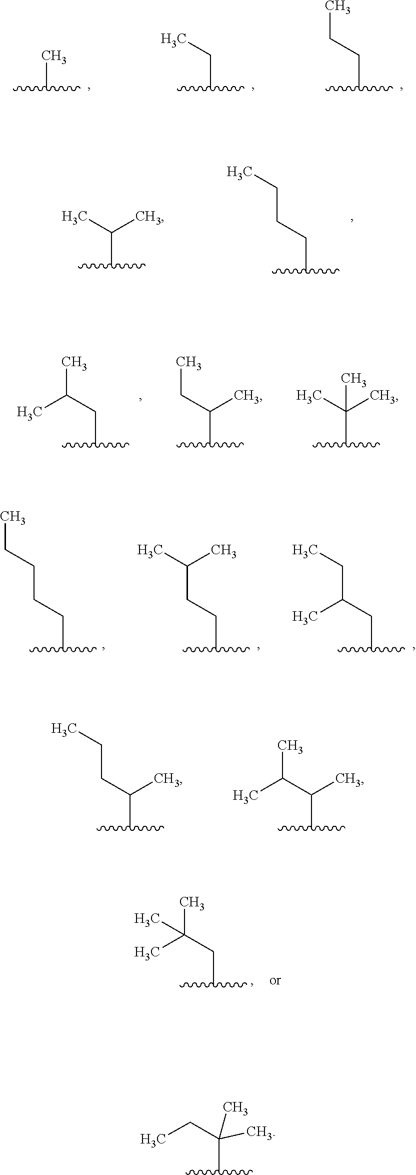

In an aspect, the structural lipid of the invention features a compound having the structure of Formula SX:

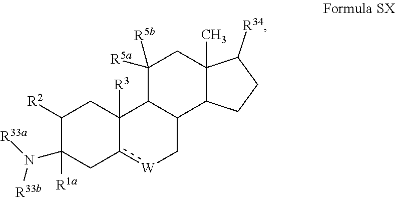

where
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, where R^{A} is H or optionally substituted C₁-C₆ alkyl;
R³ is H or

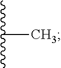

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

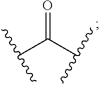 [SMILES: *C(*)=O]

R^{33a} is optionally substituted C₁-C₆ alkyl or

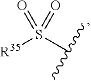

where R³⁵ is optionally substituted C₁-C₆ alkyl or optionally substituted C₆-C₁₀ aryl;
R^{33b} is H or optionally substituted C₁-C₆ alkyl; or
R³⁵ and R^{33b}, together with the atom to which each is attached, form an optionally substituted C₃-C₉ heterocyclyl; and
R³⁴ is optionally substituted C₁-C₆ alkyl or optionally substituted C₁-C₆ heteroalkyl, or a pharmaceutically acceptable salt thereof.

In some embodiments, the compound has the structure of Formula SXa:

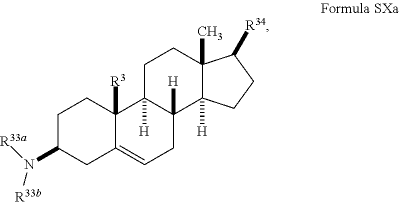

or a pharmaceutically acceptable salt thereof.

In some embodiments, the compound has the structure of Formula SXb:

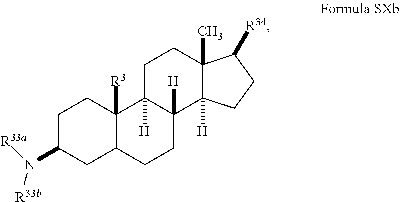

or a pharmaceutically acceptable salt thereof.

In some embodiments, R^{33a} is

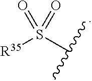

In some embodiments, R³⁵ is

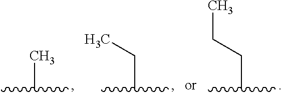

In some embodiments, R³⁵ is

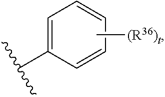

where
t is 0, 1, 2, 3, 4, or 5; and
each R³⁶ is, independently, halo, hydroxyl, optionally substituted C₁-C₆ alkyl, or optionally substituted C₁-C₆ heteroalkyl.

In some embodiments, R³⁴ is

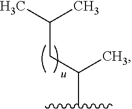

where u is 0, 1, 2, 3, or 4.

In some embodiments, u is 3 or 4.

In an aspect, the structural lipid of the invention features a compound having the structure of Formula SXI:

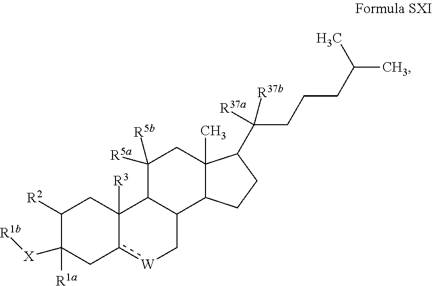

where
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, where R^{A} is H or optionally substituted C₁-C₆ alkyl;
R³ is H or

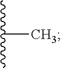

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

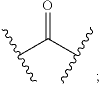 [SMILES: *C(*)=O]

and
each of R^{37a} and R^{37b} is, independently, optionally substituted C₁-C₆ alkyl, optionally substituted C₁-C₆ heteroalkyl, halo, or hydroxyl,
   or a pharmaceutically acceptable salt thereof.

In some embodiments, the compound has the structure of Formula SXIa:

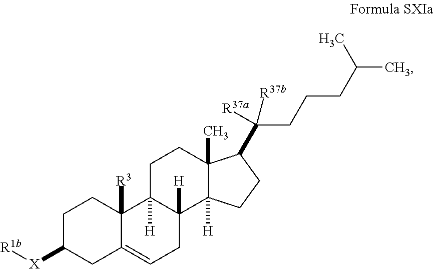

or a pharmaceutically acceptable salt thereof.

In some embodiments, the compound has the structure of Formula SXIb:

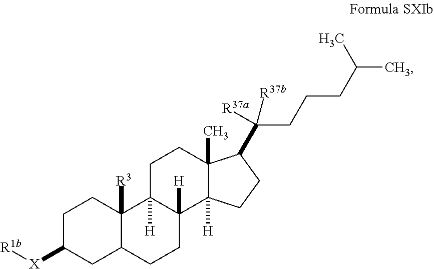

or a pharmaceutically acceptable salt thereof.

In some embodiments, R^{37a} is hydroxyl.

In some embodiments, R^{37b} is

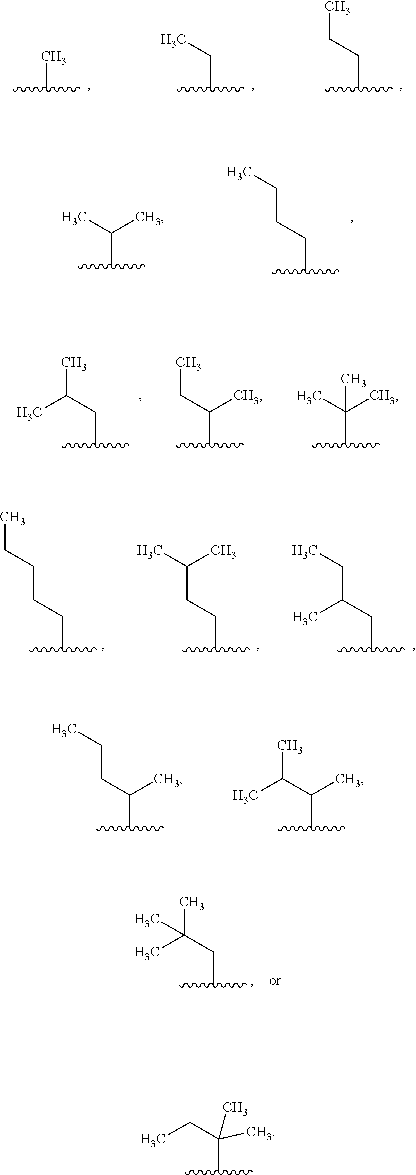

In an aspect, the structural lipid of the invention features a compound having the structure of Formula SXII:

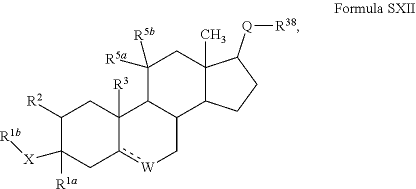

where
R^{1a} is H, optionally substituted C₁-C₆ alkyl, optionally substituted C₂-C₆ alkenyl, or optionally substituted C₂-C₆ alkynyl;
X is O or S;
R² is H or OR^{A}, where R^{A} is H or optionally substituted C₁-C₆ alkyl;
R³ is H or

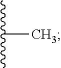

[symbol] represents a single bond or a double bond;
W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};
each of R^{4a} and R^{4b} is, independently, H, halo, or optionally substituted C₁-C₆ alkyl;
each of R^{5a} and R^{5b} is, independently, H or OR^{A}, or R^{5a} and R^{5b}, together with the atom to which each is attached, combine to form

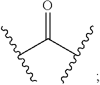 [SMILES: *C(*)=O]

and
Q is O, S, or NR^{E}, where R^{E} is H or optionally substituted C₁-C₆ alkyl; and
R³⁸ is optionally substituted C₁-C₆ alkyl,
   or a pharmaceutically acceptable salt thereof.

In some embodiments, the compound has the structure of Formula SXIIa:

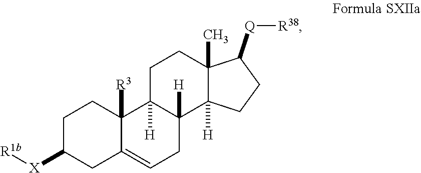

or a pharmaceutically acceptable salt thereof.

In some embodiments, the compound has the structure of Formula SXIIb:

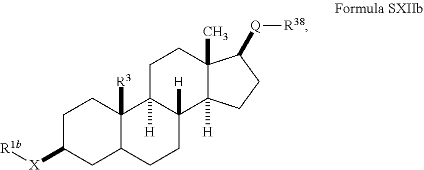

or a pharmaceutically acceptable salt thereof.

In some embodiments, Q is NR^{E}.

In some embodiments, R^{E} is H or

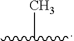

In some embodiments, R^{E} is H. In some embodiments, R^{E} is

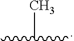

In some embodiments, R³⁸ is

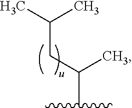

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

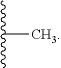

In some embodiments, R^{5a} is H.

In some embodiments, R^{5b} is H.

In an aspect, the invention features a compound having the structure of any one of compounds S-1-42, S-150, S-154, S-162-165, S-169-172 and S-184 in Table 1, or any pharmaceutically acceptable salt thereof. As used herein, “CMPD” refers to “compound.”

TABLE 1
Compounds of Formula SI

| CMPD | |
|---|---|
| No. S- | Structure |
| 1 | 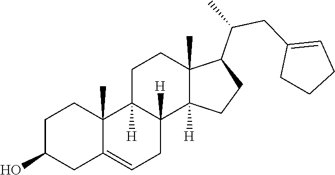 |
| 2 | 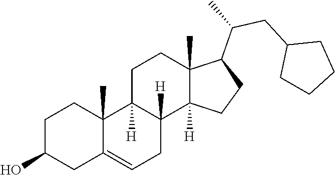 |
| 3 | 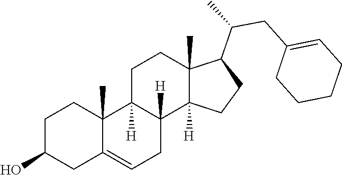 |
| 4 | 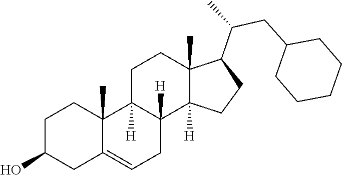 |
| 5 | 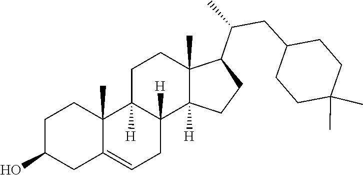 |
| 6 | 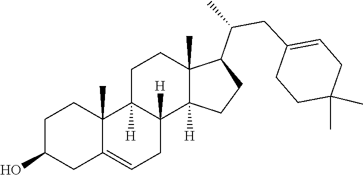 |
| 7 | 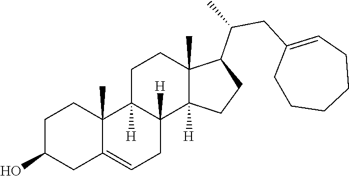 |
| 8 | 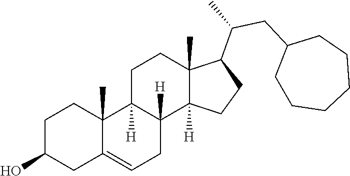 |
| 9 | 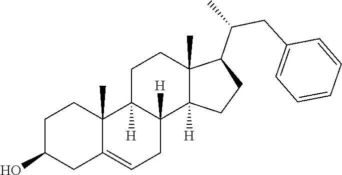 |
| 10 | 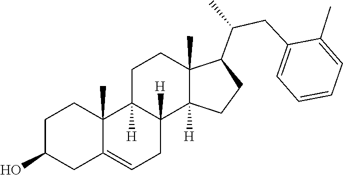 |
| 11 | 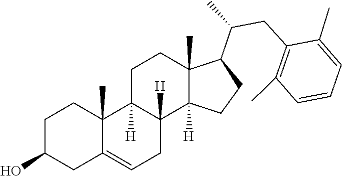 |
| 12 | 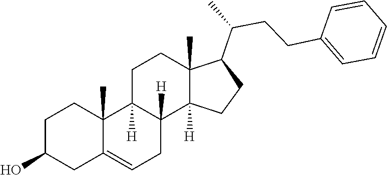 |
| 13 | 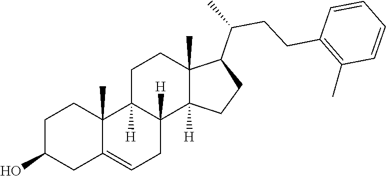 |
| 14 | 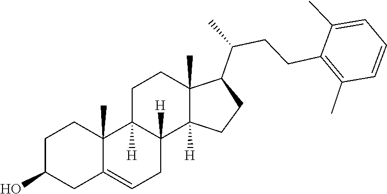 |
| 15 | 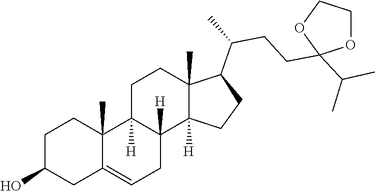 |
| 16 | 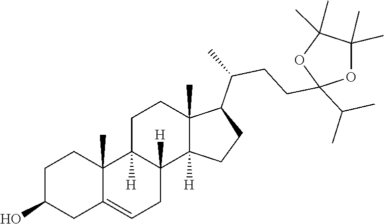 |
| 17 | 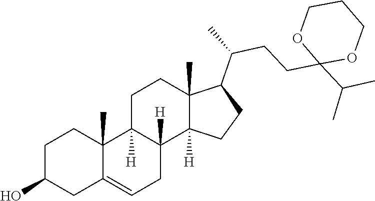 |
| 18 | 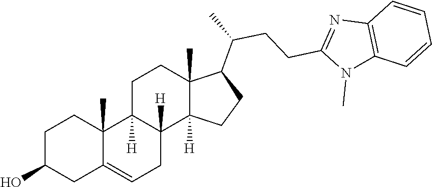 |
| 19 | 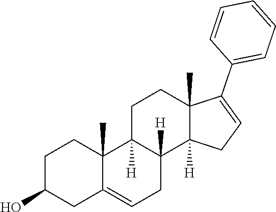 |
| 20 | 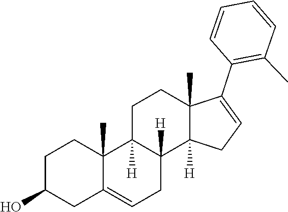 |
| 21 | 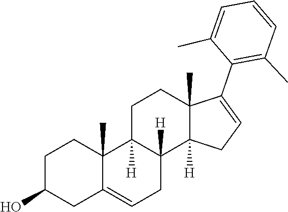 |
| 22 | 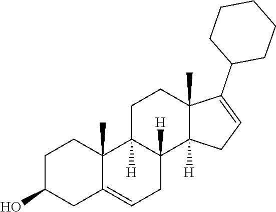 |
| 23 | 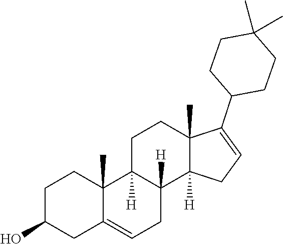 |
| 24 | 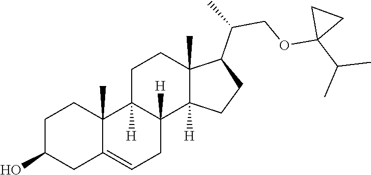 |
| 25 | 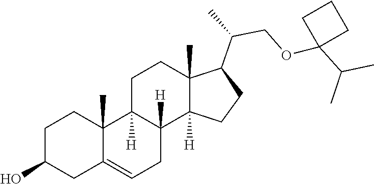 |
| 26 | 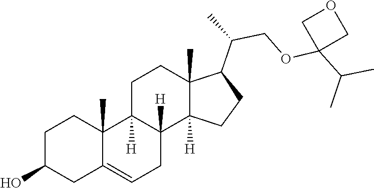 |
| 27 | 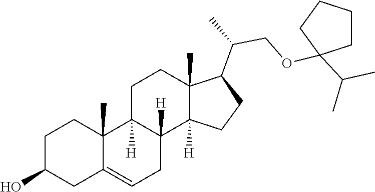 |
| 28 | 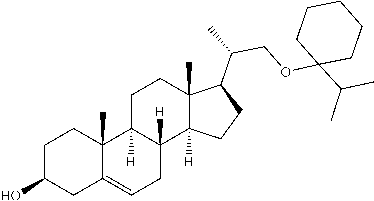 |
| 29 | 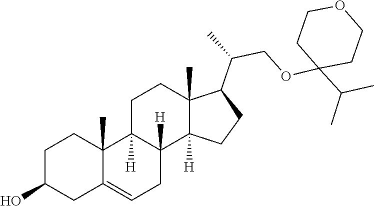 |
| 30 | 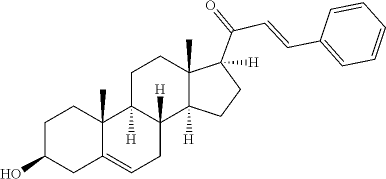 |
| 31 | 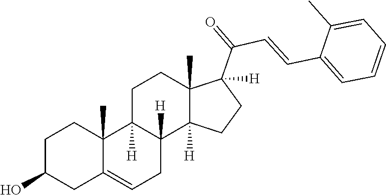 |
| 32 | 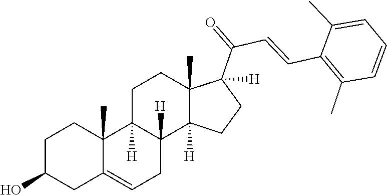 |
| 33 | 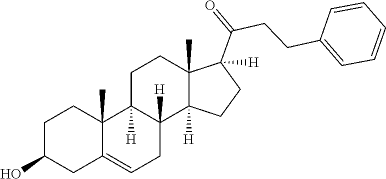 |
| 34 | 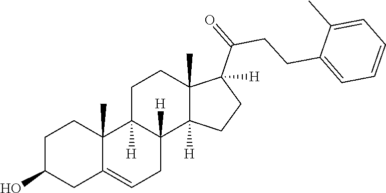 |
| 35 | 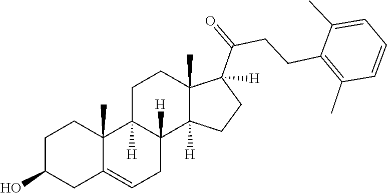 |
| 36 | 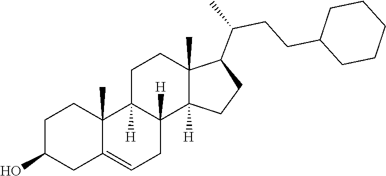 |
| 37 | 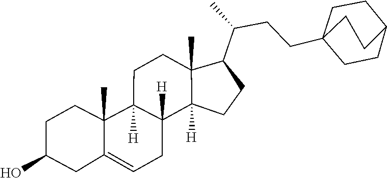 |
| 38 | 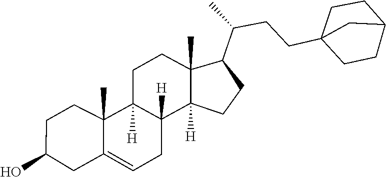 |
| 39 | 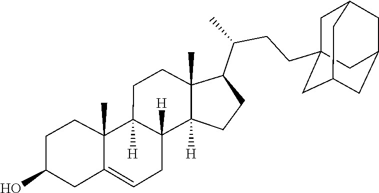 |
| 40 | 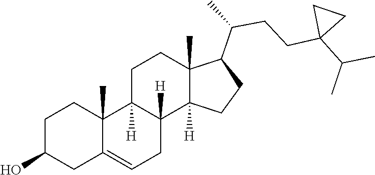 |
| 41 | 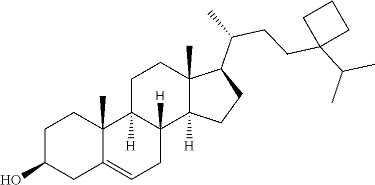 |
| 42 | 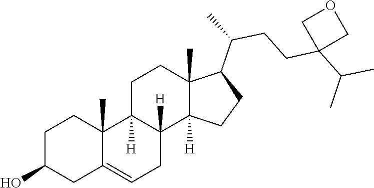 |
| 150 | 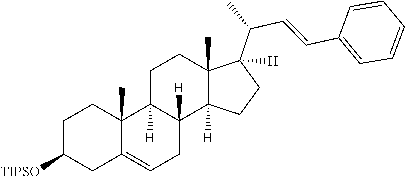 |
| 154 | 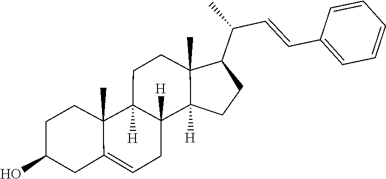 |
| 162 | 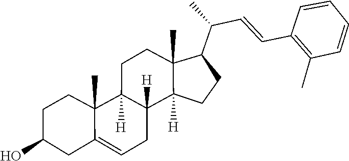 |
| 163 | 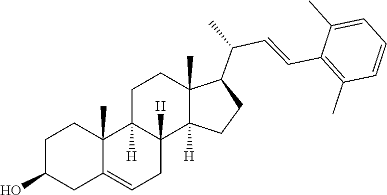 |
| 164 | 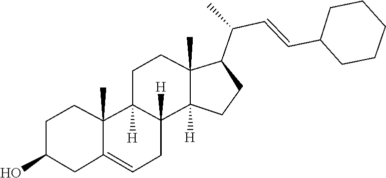 |
| 165 | 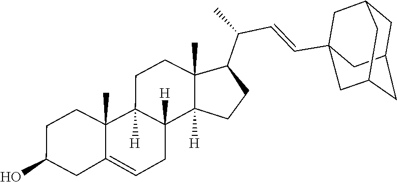 |
| 169 | 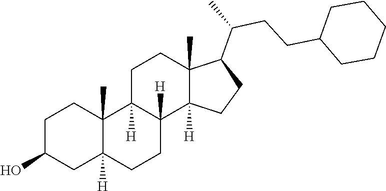 |
| 170 | 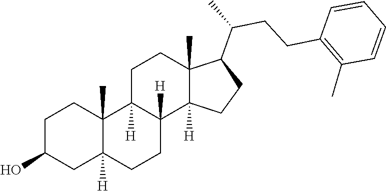 |
| 171 | 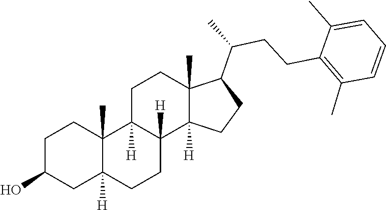 |
| 172 | 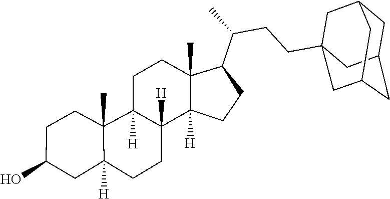 |
| 184 | 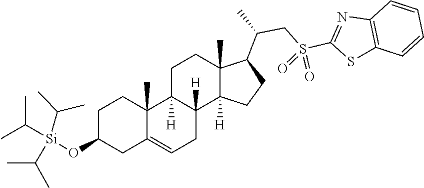 |

In an aspect, the invention features a compound having the structure of any one of compounds S-43-50 and S-175-178 in Table 2, or any pharmaceutically acceptable salt thereof.

TABLE 2
Compounds of Formula SII

| CMPD | |
|---|---|
| No. | |
| S— | Structure |
| 43 | 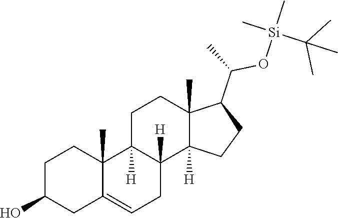 |
| 44 | 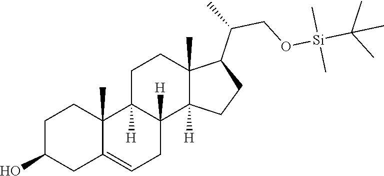 |
| 45 | 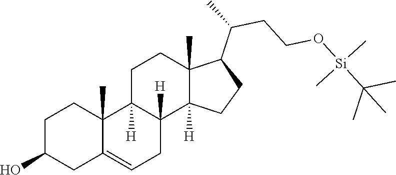 |
| 46 | 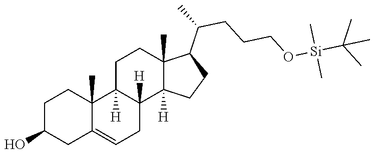 |
| 175 | 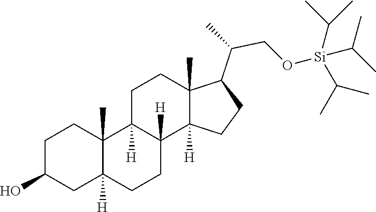 |
| 176 | 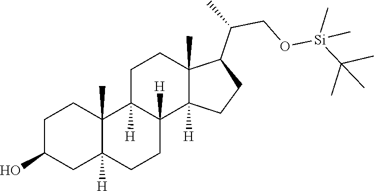 |
| 47 | 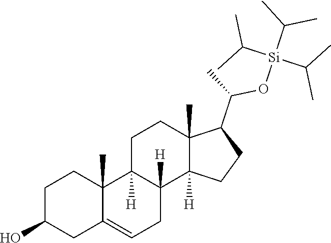 |
| 48 | 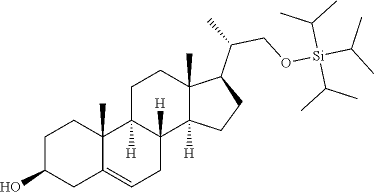 |
| 49 | 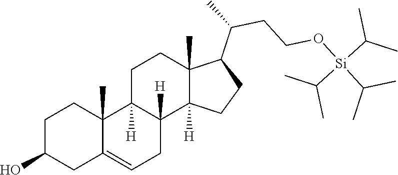 |
| 50 | 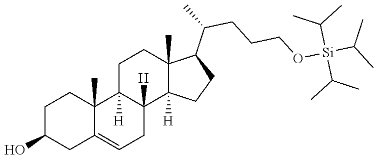 |
| 177 | 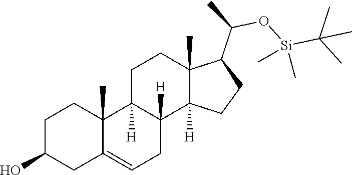 |
| 178 | 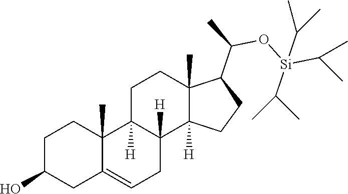 |

In an aspect, the invention features a compound having the structure ofany one of compounds S-1-67, S-149 and S-153 in Table 3, or any pharmaceutically acceptable salt thereof.

TABLE 3
Compounds of Formula SIII

| CMPD | |
|---|---|
| No. | |
| S— | Structure |
| 51 | 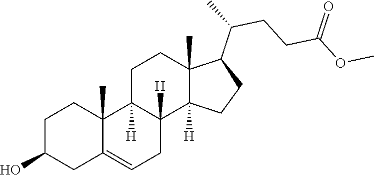 |
| 52 | 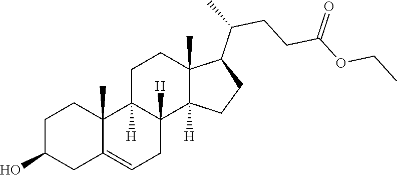 |
| 53 | 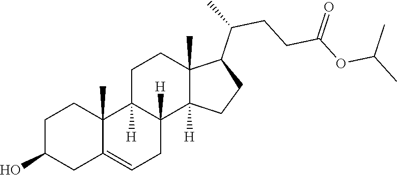 |
| 54 | 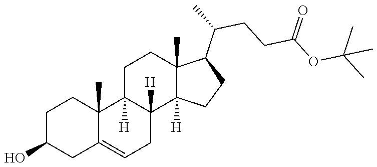 |
| 55 | 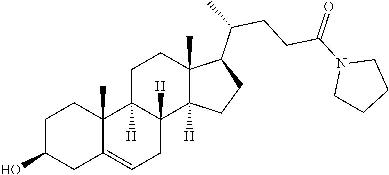 |
| 56 | 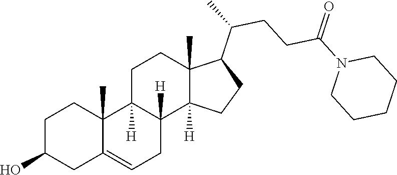 |
| 57 | 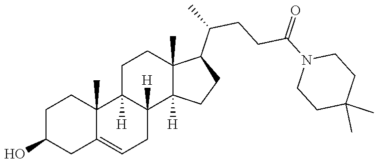 |
| 58 | 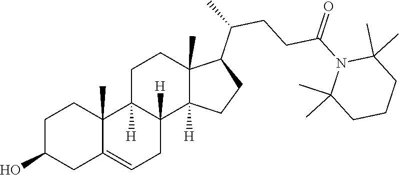 |
| 59 | 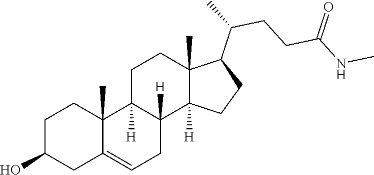 |
| 153 | 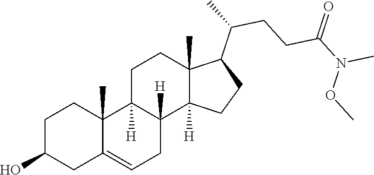 |
| 60 | 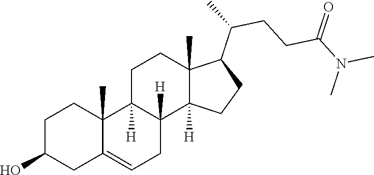 |
| 61 | 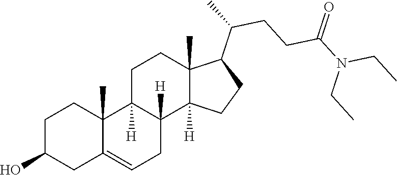 |
| 62 | 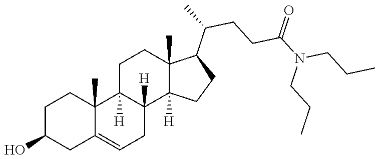 |
| 63 | 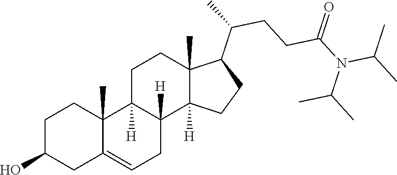 |
| 64 | 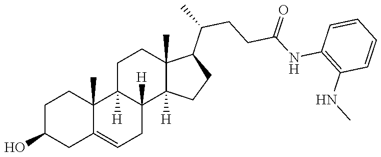 |
| 65 | 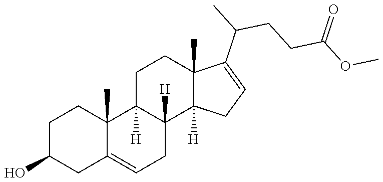 |
| 66 | 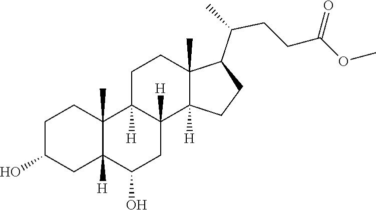 |
| 67 | 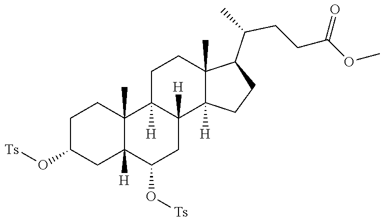 |
| 149 | 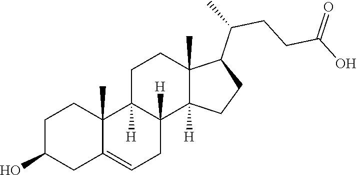 |

In an aspect, the invention features a compound having the structure of any one of compounds S-68-73 in Table 4, or any pharmaceutically acceptable salt thereof.

TABLE 4
Compounds of Formula SIV

| CMPD | |
|---|---|
| No. S— | Structure |
| 68 | 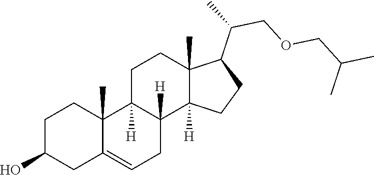 |
| 69 | 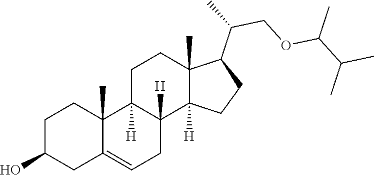 |
| 70 | 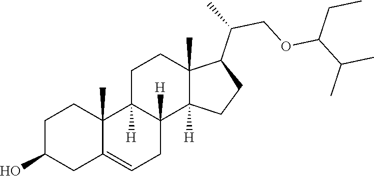 |
| 71 | 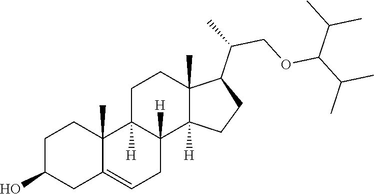 |
| 72 | 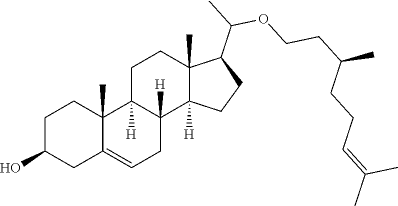 |
| 73 | 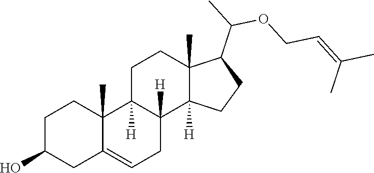 |

In an aspect, the invention features a compound having the structure of any one of compounds S-74-78 in Table 5, or any pharmaceutically acceptable salt thereof.

TABLE 5
Compounds of Formula SV

| CMPD | |
|---|---|
| No. S— | Structure |
| 74 | 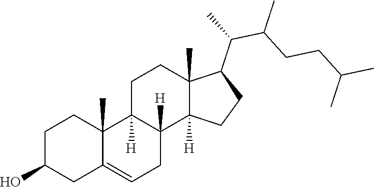 |
| 75 | 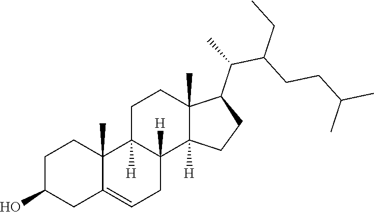 |
| 76 | 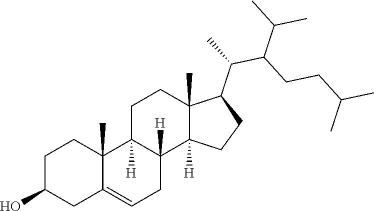 |
| 77 | 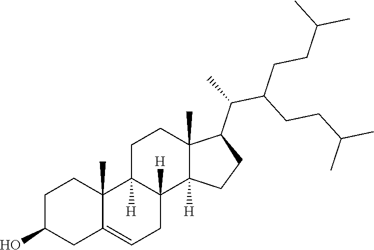 |
| 78 | 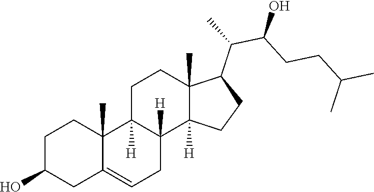 |

In an aspect, the invention features a compound having the structure of any one of compounds S-79 or S-80 in Table 6, or any pharmaceutically acceptable salt thereof.

TABLE 6
Compounds of Formula SVI

| CMPD | |
|---|---|
| No. S— | Structure |
| 79 | 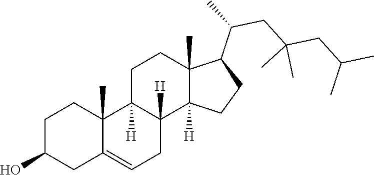 |
| 80 | 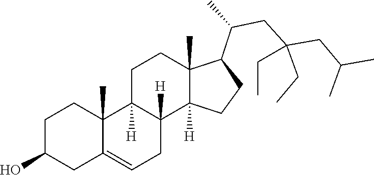 |

In an aspect, the invention features a compound having the structure of any one of compounds S-81-87, S-152 and S-157 in Table 7, or any pharmaceutically acceptable salt thereof.

TABLE 7
Compounds of Formula S-VII

| CMPD | |
|---|---|
| No. S— | Structure |
| 81 | 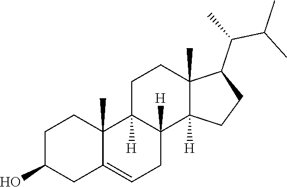 |
| 82 | 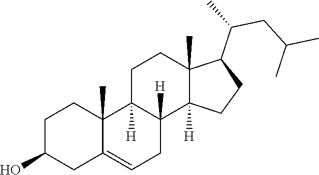 |
| 83 | 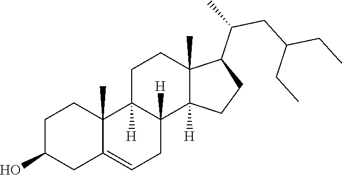 |
| 84 | 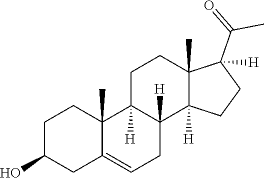 |
| 157 | 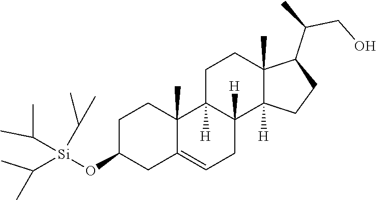 |
| 85 | 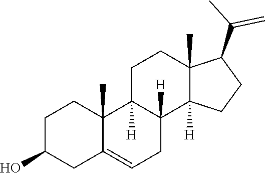 |
| 86 | 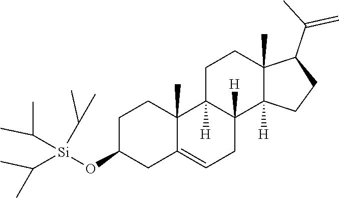 |
| 87 | 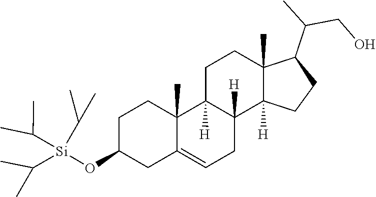 |
| 152 | 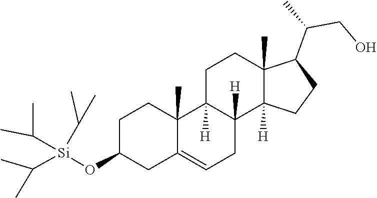 |

In an aspect, the invention features a compound having the structure of any one of compounds S-88-97 in Table 8, or any pharmaceutically acceptable salt thereof.

TABLE 8
Compounds of Formula SVIII

| CMPD | |
|---|---|
| No. S— | Structure |
| 88 | 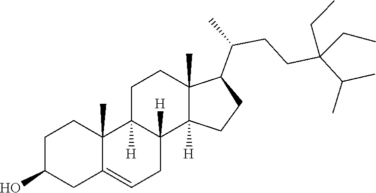 |
| 89 | 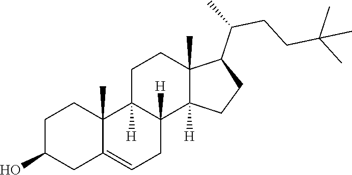 |
| 90 | 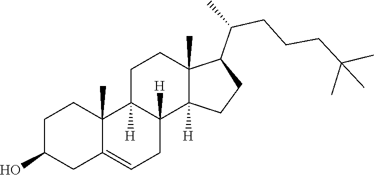 |
| 91 | 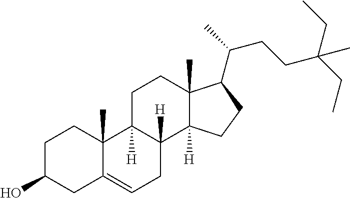 |
| 92 | 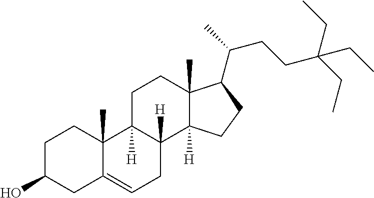 |
| 93 | 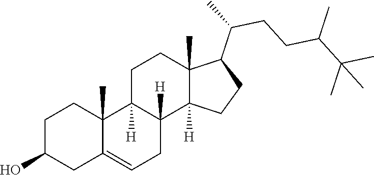 |
| 94 | 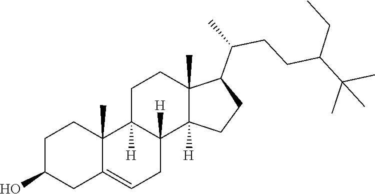 |
| 95 | 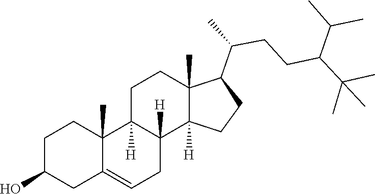 |
| 96 | 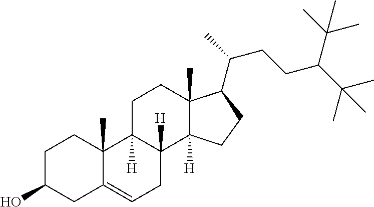 |
| 97 | 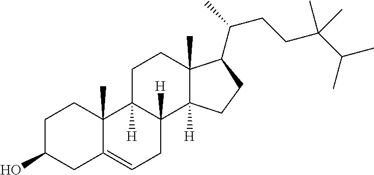 |

In an aspect, the invention features a compound having the structure of any one of compounds S-98-105 and S-180-182 in Table 9, or any pharmaceutically acceptable salt thereof.

TABLE 9
Compounds of Formula SIX

| CMPD | |
|---|---|
| No. S— | Structure |
| 98 | 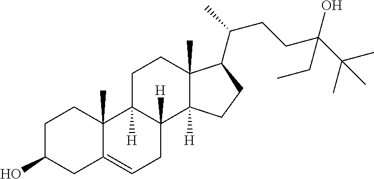 |
| 99 | 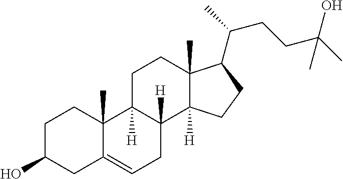 |
| 100 | 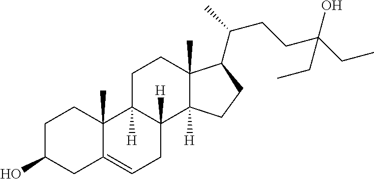 |
| 101 | 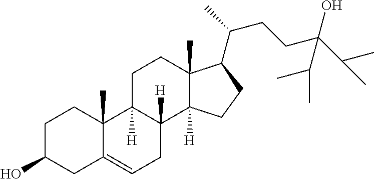 |
| 180 | 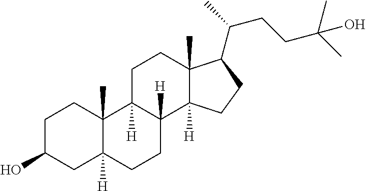 |
| 181 | 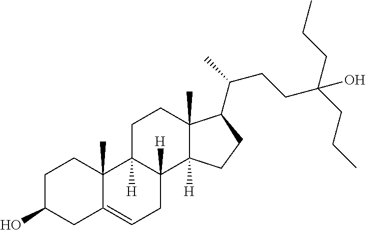 |
| 102 | 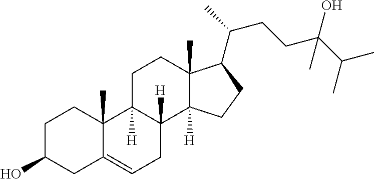 |
| 103 | 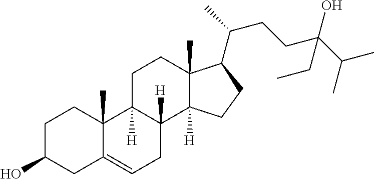 |
| 104 | 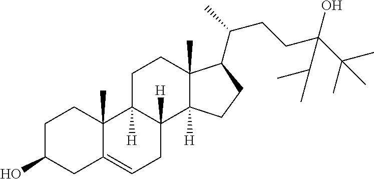 |
| 105 | 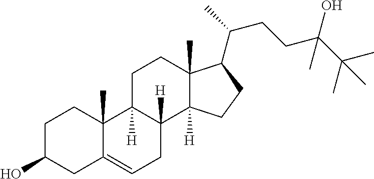 |
| 182 | 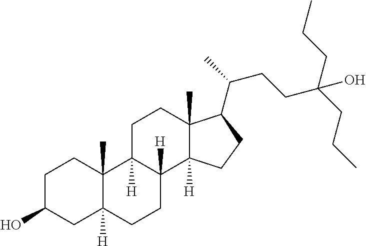 |

In an aspect, the invention features a compound having the structure of compound S-106 in Table 10, or any pharmaceutically acceptable salt thereof.

TABLE 10
Compounds of Formula SX

| CMPD | |
|---|---|
| No. | |
| S— | Structure |
| 106 | 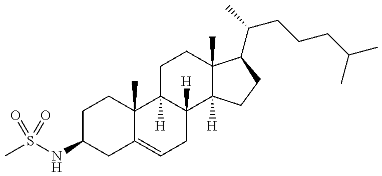 |

In an aspect, the invention features a compound having the structure of compound S-107 or S-108 in Table 11, or any pharmaceutically acceptable salt thereof.

TABLE 11
Compounds of Formula SXI

| CMPD | |
|---|---|
| No. S— | Structure |
| 107 | 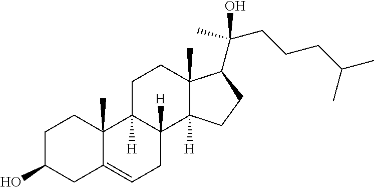 |
| 108 | 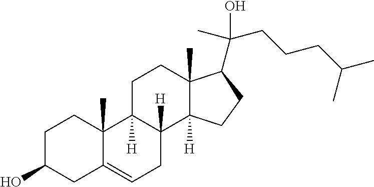 |

In an aspect, the invention features a compound having the structure of compound S-109 in Table 12, or any pharmaceutically acceptable salt thereof.

TABLE 12
Compounds of Formula SXII

| CMPD | |
|---|---|
| No. S— | Structure |
| 109 | 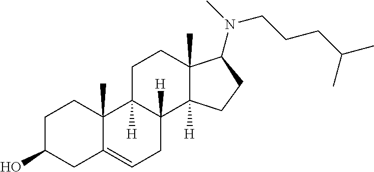 |

In an aspect, the invention features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Invention

| CMPD | |
|---|---|
| No. | |
| S— | Structure |
| 110 | 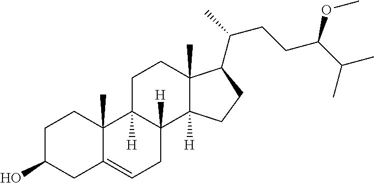 |
| 111 | 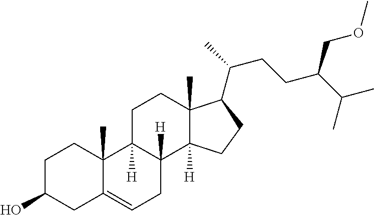 |
| 112 | 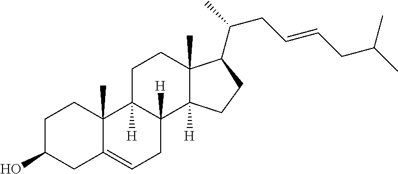 |
| 113 | 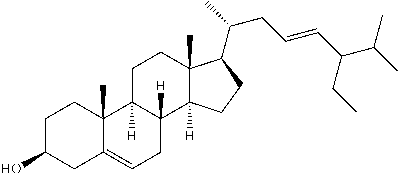 |
| 114 | 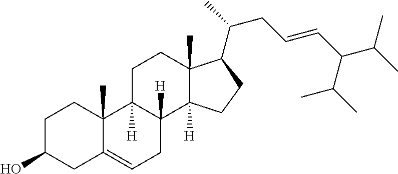 |
| 115 | 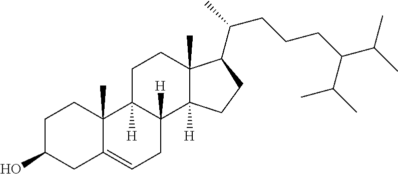 |
| 116 | 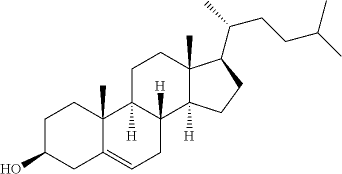 |
| 117 | 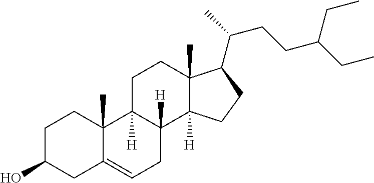 |
| 118 | 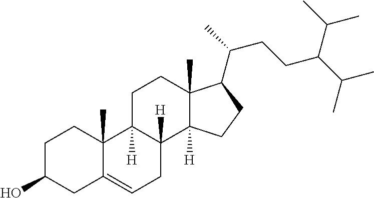 |
| 119 | 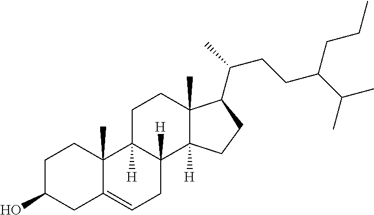 |
| 120 | 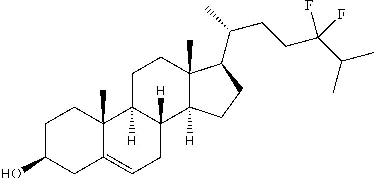 |
| 156 | 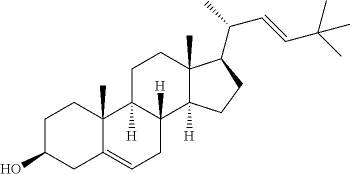 |
| 158 | 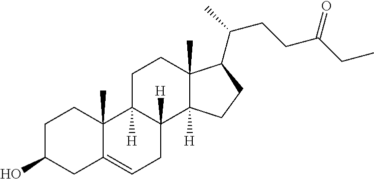 |
| 160 | 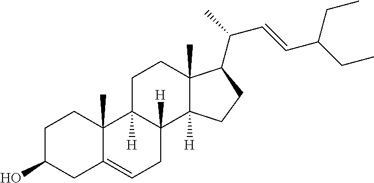 |
| 161 | 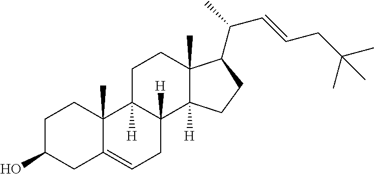 |
| 166 | 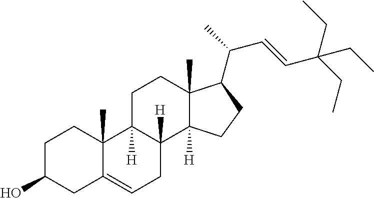 |
| 121 | 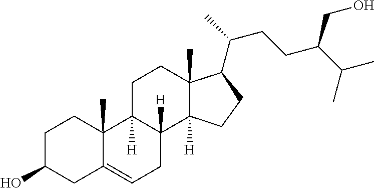 |
| 122 | 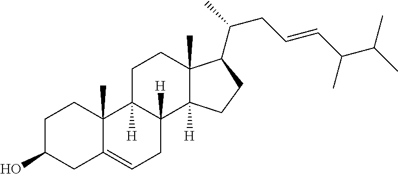 |
| 123 | 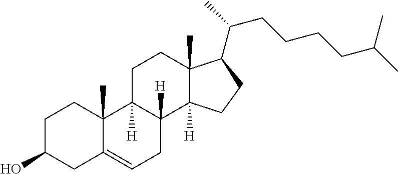 |
| 124 | 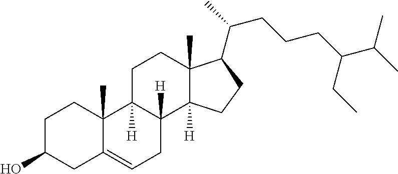 |
| 125 | 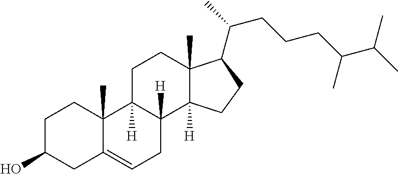 |
| 126 | 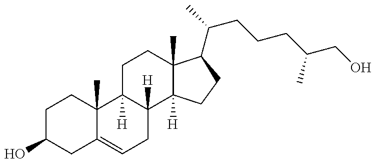 |
| 127 | 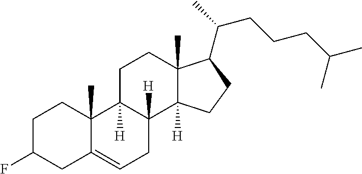 |
| 128 | 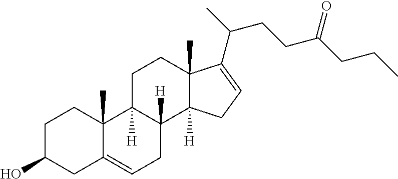 |
| 129 | 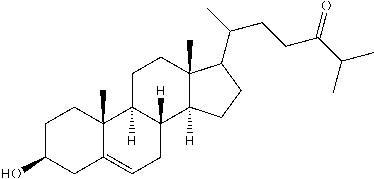 |
| 130 | 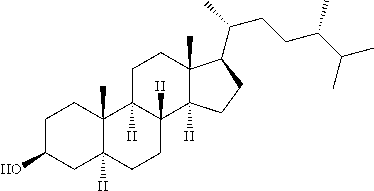 |
| 155 | 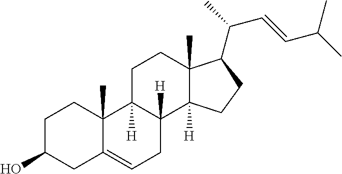 |
| 167 | 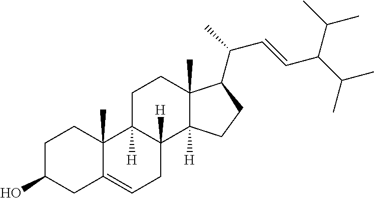 |
| 168 | 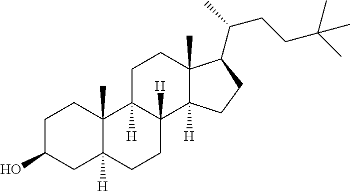 |
| 173 | 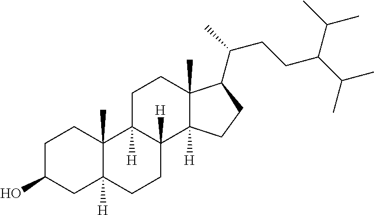 |
| 174 | 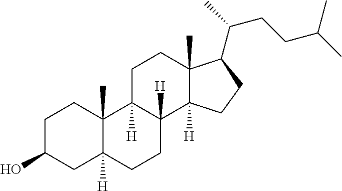 |
| 179 | 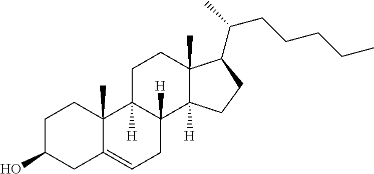 |

In an aspect, the invention features a compound having the structure of any one of compounds S-131-133 in Table 14, or any pharmaceutically acceptable salt thereof.

TABLE 14
Compounds of the Invention

| CMPD | |
|---|---|
| No. S— | Structure |
| 131 | 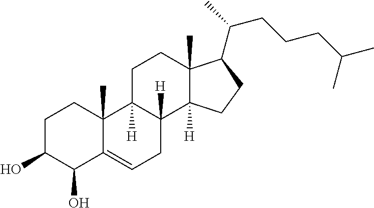 |
| 132 | 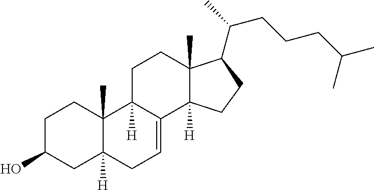 |
| 133 | 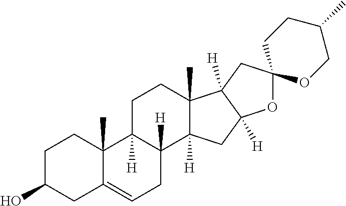 |

In an aspect, the invention features a compound having the structure of any one of compounds S-134-148, S-151 and S-159 in Table 15, or any pharmaceutically acceptable salt thereof.

TABLE 15
Compounds of the Invention

| CMPD | |
|---|---|
| No. S— | Structure |
| 134 | 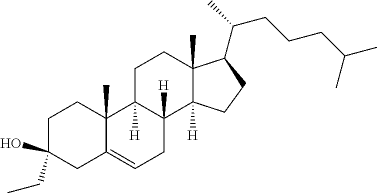 |
| 135 | 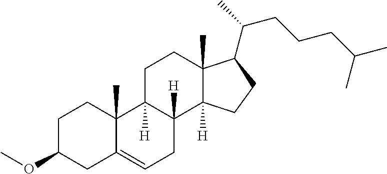 |
| 136 | 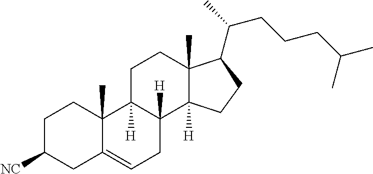 |
| 137 | 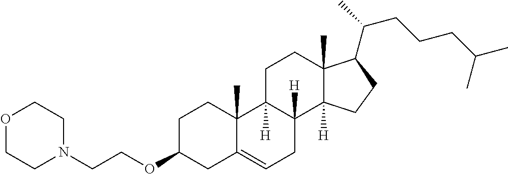 |
| 138 | 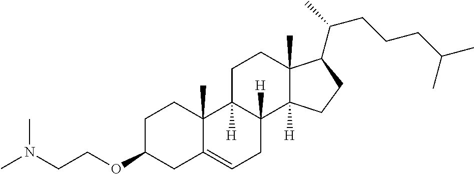 |
| 139 | 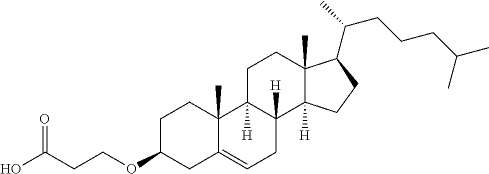 |
| 140 | 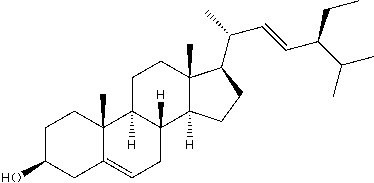 |
| 141 | 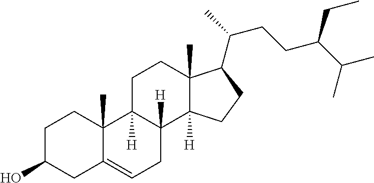 |
| 159 | 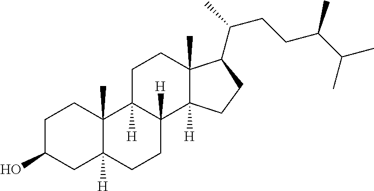 |
| 142 | 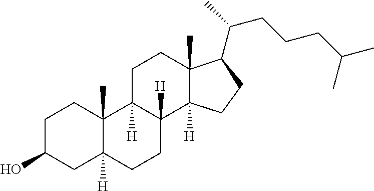 |
| 143 | 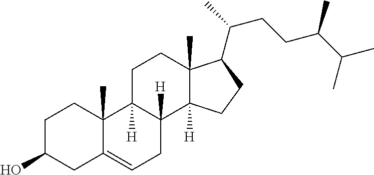 |
| 144 | 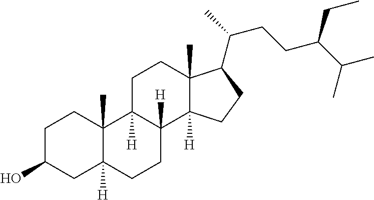 |
| 145 | 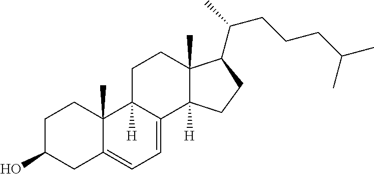 |
| 146 | 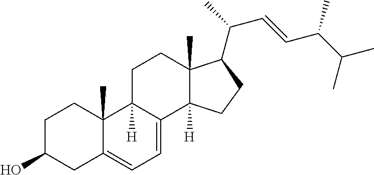 |
| 147 | 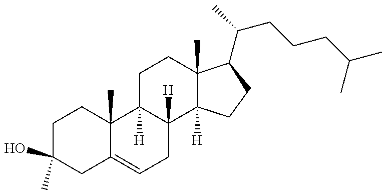 |
| 148 | 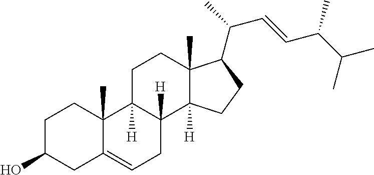 |
| 151 | 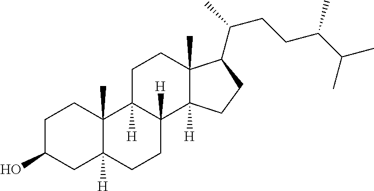 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one or more structural lipids of the lipid nanoparticles of the invention can be composition 183 in Table 16.

TABLE 16
Structural Lipid Compositions

| Composi- | |
|---|---|
| tion | |
| S— No. | Structure |
| 183 | 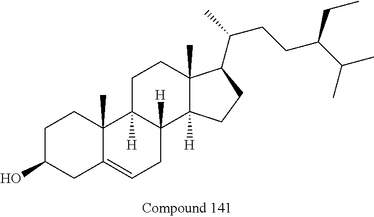 |
| | 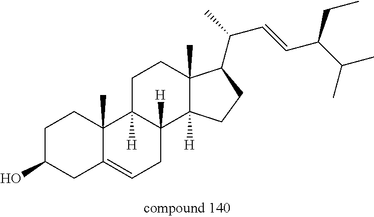 |
| | 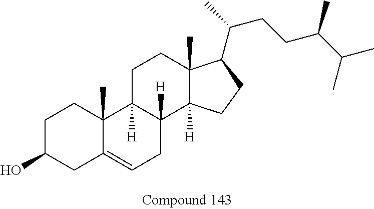 |
| | 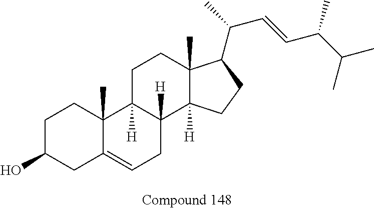 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

### Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds S-1-148 combined with a cholesterol and/or a phytosterol.

For example, the structural component of the lipid nanoparticle can be a mixture of one or more structural compounds (e.g. any of Compounds S-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.054.95% of the sterols in the composition.

### Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

### Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

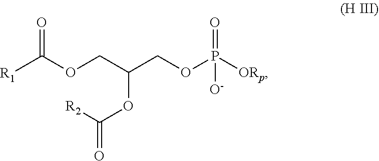

in which Rp represents a phospholipid moiety and R1 and R2 represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.
Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE (18:2/18:2), 1,2-dilinolenoyl-sn-glycero-3-phosphoethanolamine (PE 18:3 (9Z, 12Z, 15Z), 1,2-diarachidonoyl-sn-glycero-3-phosphoethanolamine (DAPE 18:3 (9Z, 12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin. Each possibility represents a separate embodiment of the invention.
In some embodiments, a LNP includes DSPC. In certain embodiments, a LNP includes DOPE.
In some embodiments, a LNP includes DMPE. In some embodiments, a LNP includes both DSPC and DOPE.
In one embodiment, a non-cationic helper lipid for use in a target cell target cell delivery LNP is selected from the group consisting of: DSPC, DMPE, and DOPC or combinations thereof. Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

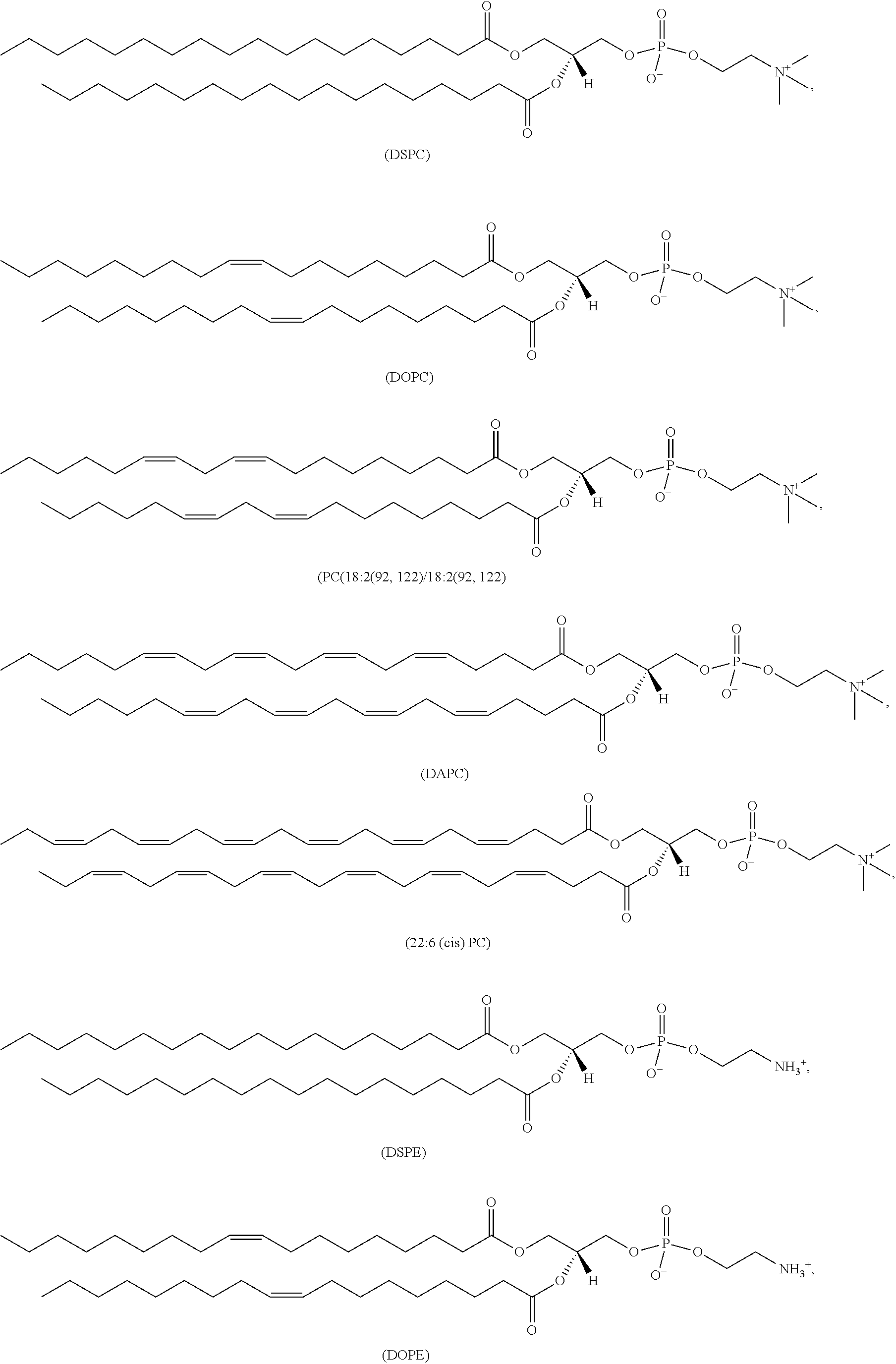

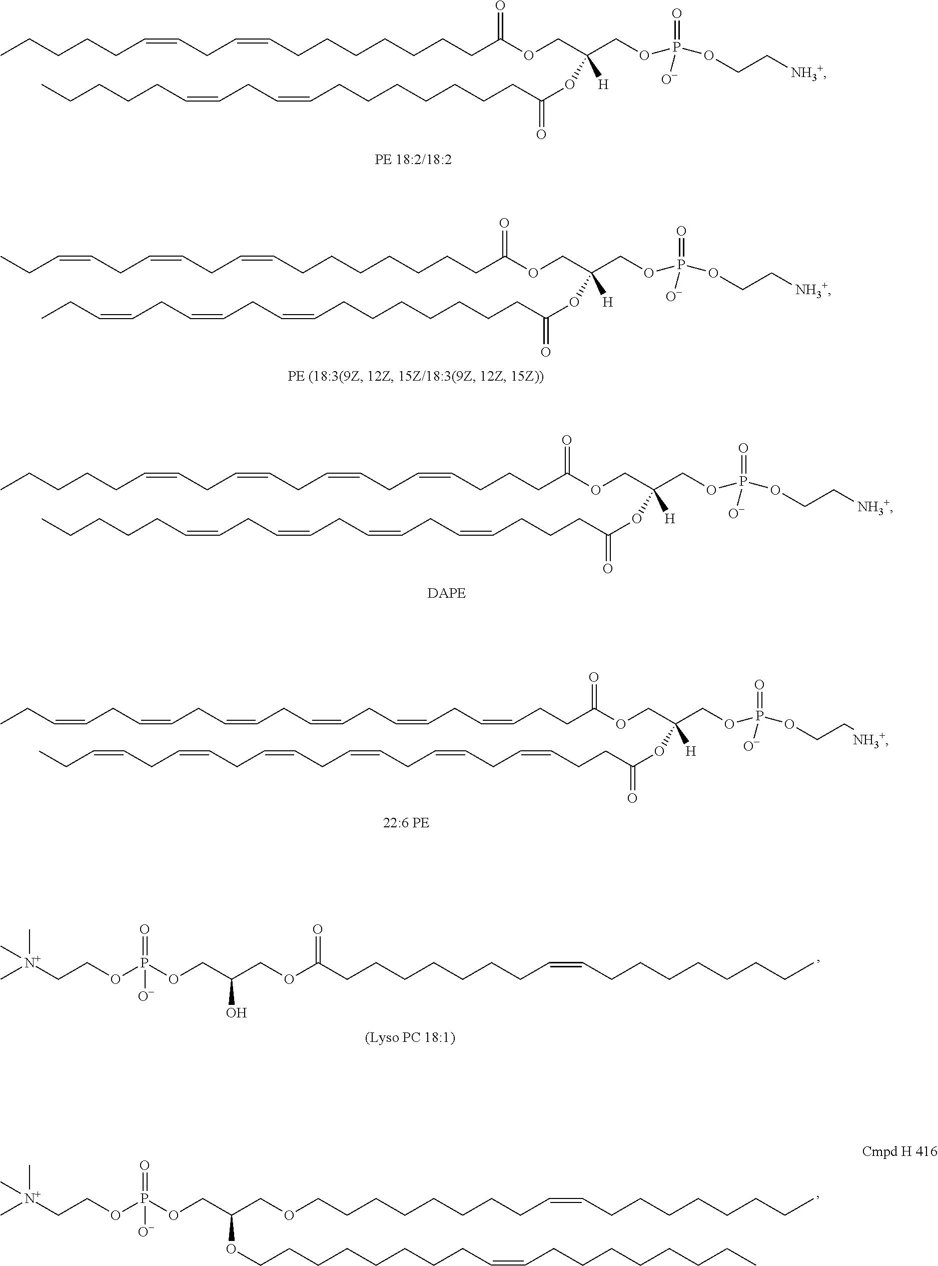

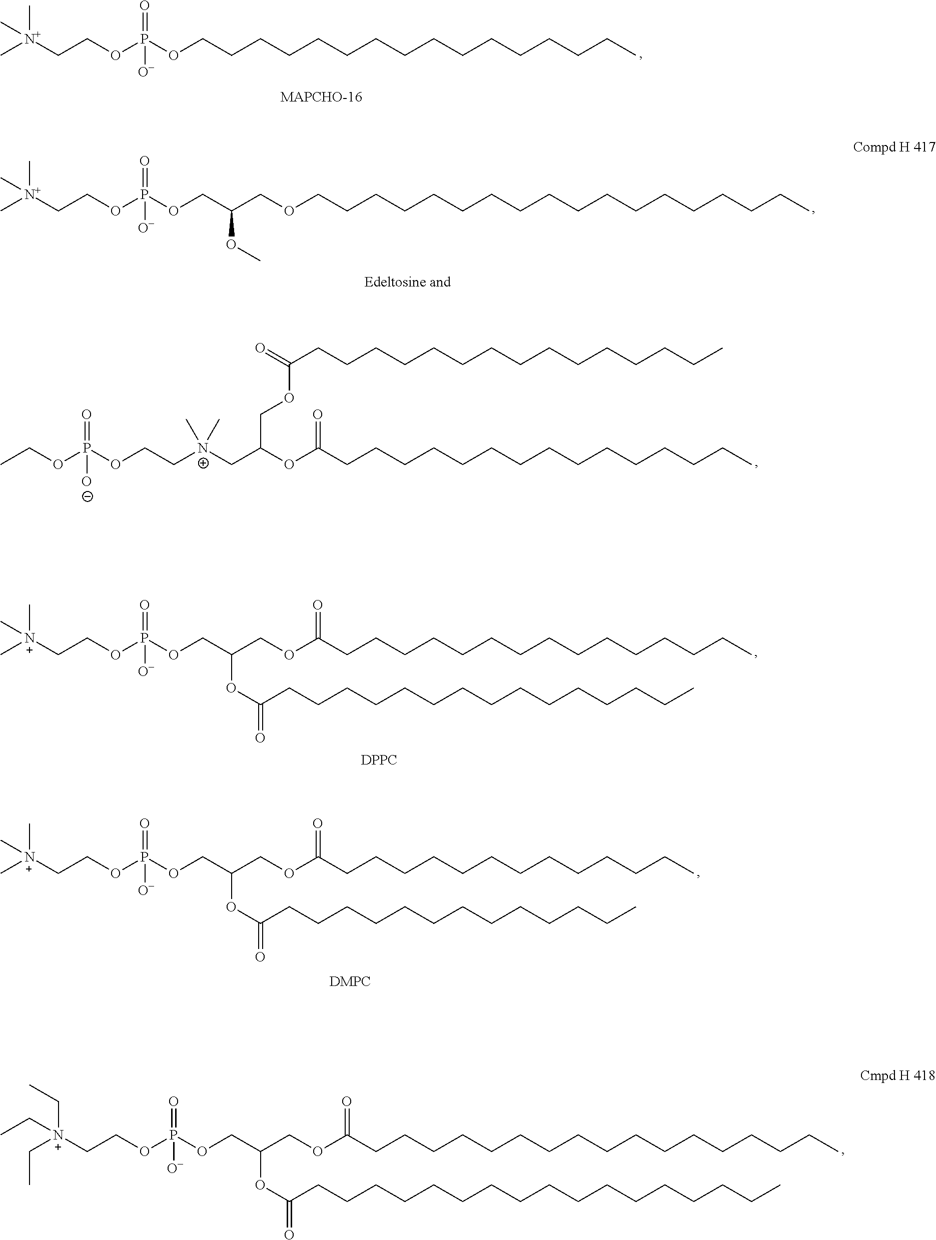

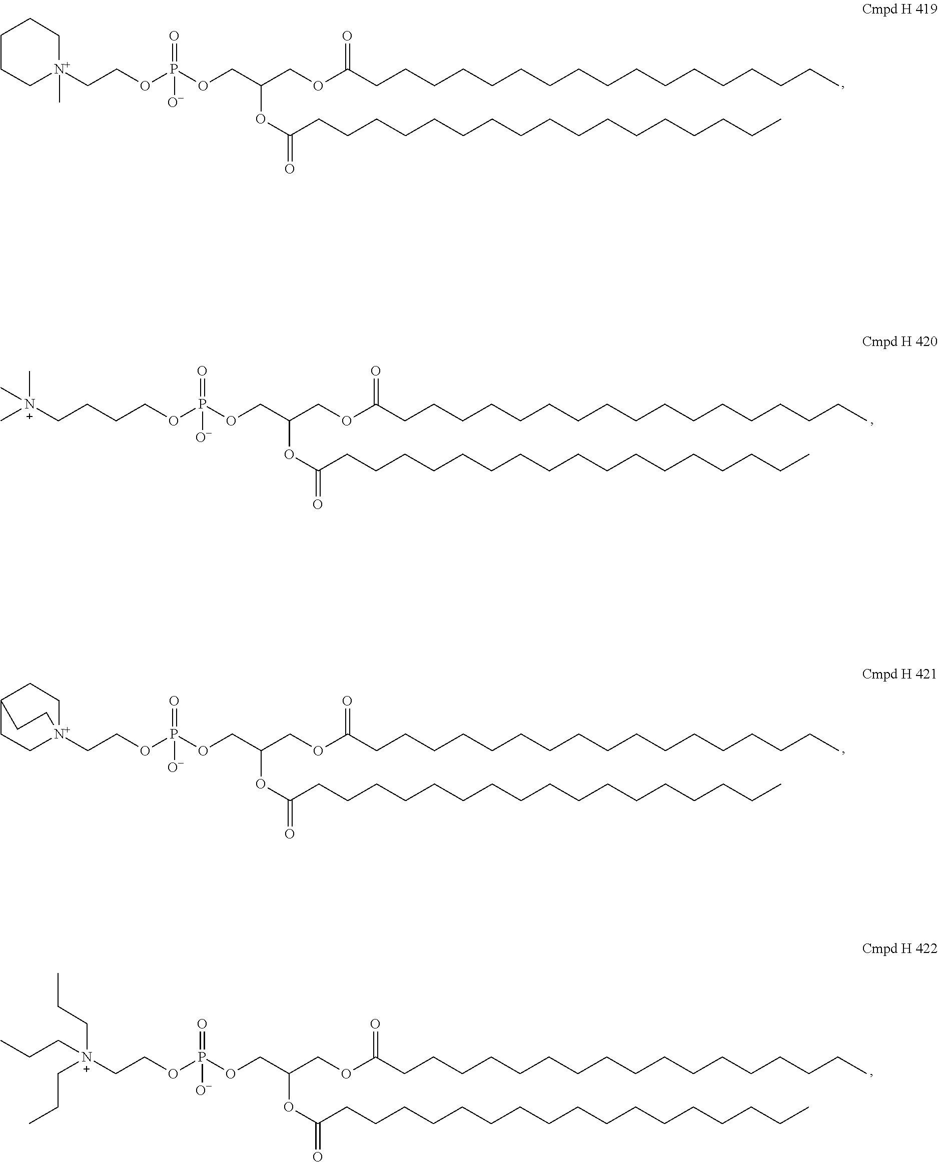

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

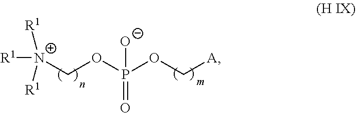

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

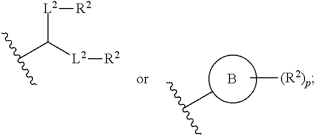

each instance of L² is independently a bond or optionally substituted C₁₋₆ alkylene, wherein one methylene unit of the optionally substituted C₁₄ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

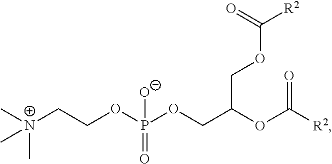

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

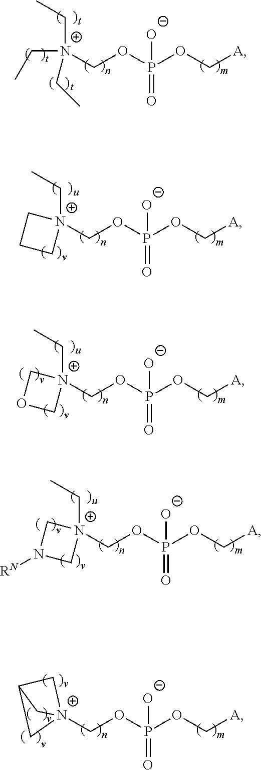

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (H IX) is of one of the following formulae:

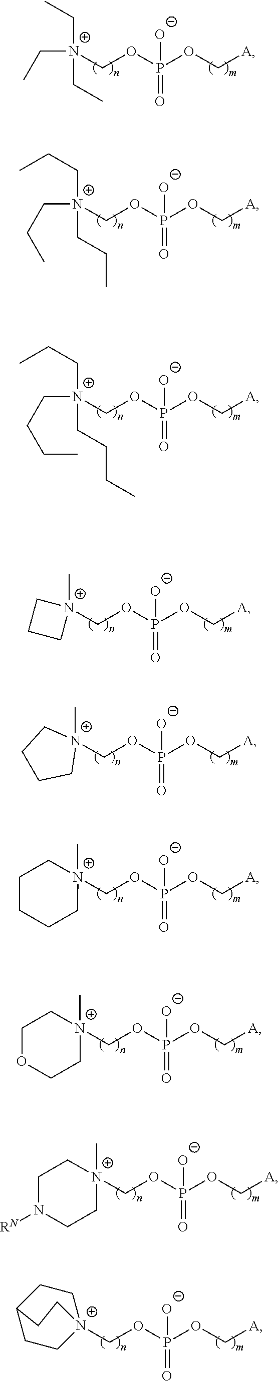

or a salt thereof.

In certain embodiments, a compound of Formula (H IX) is one of the following:

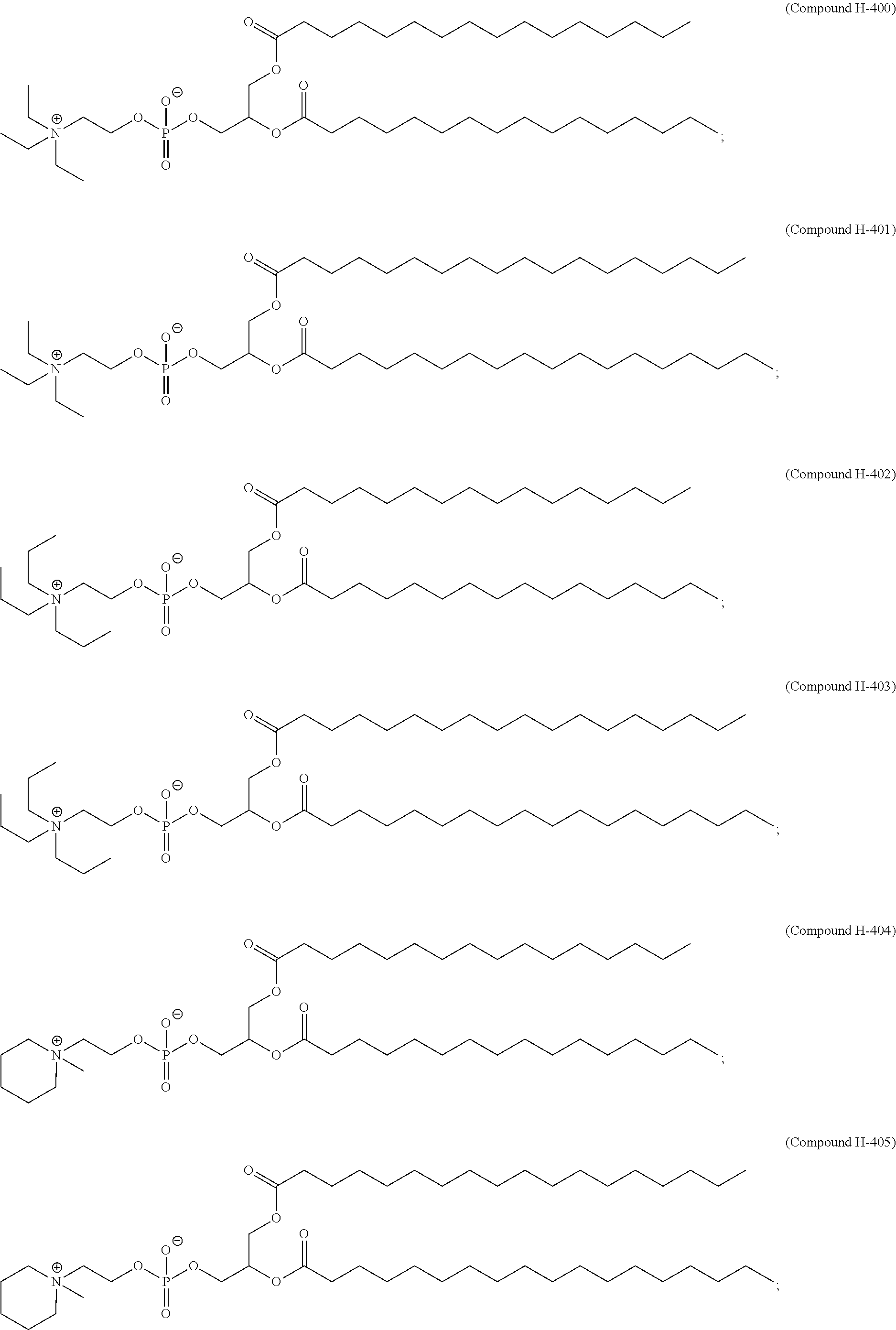

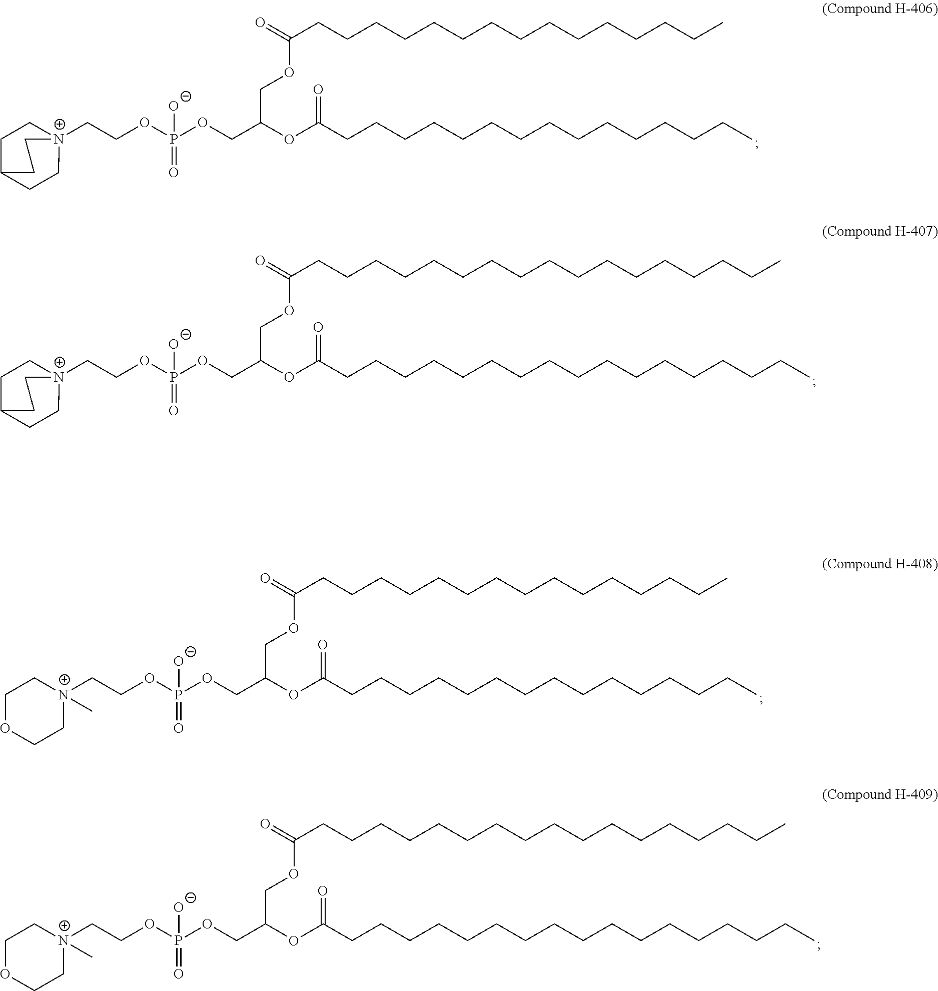 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In one embodiment, a target cell target cell delivery LNP comprises Compound H-409 as a non-cationic helper lipid.

### (ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c):

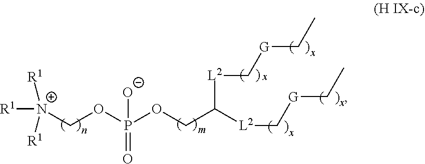

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N}), or —N(R^{N})S(O)₂O—. Each possibility represents a separate embodiment of the present invention.

In certain embodiments, the compound of Formula (H IX-c) is of Formula (H IX-c-1):

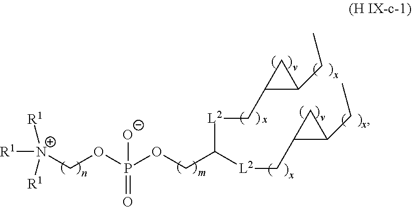

or salt thereof, wherein:
each instance of v is independently 1, 2, or 3.

In certain embodiments, the compound of Formula (H IX-c) is of Formula (H IX-c-2):

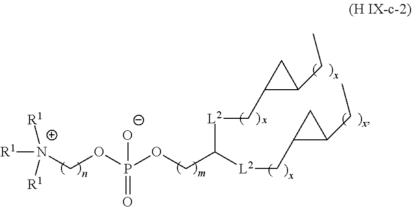

or a salt thereof.

In certain embodiments, the compound of Formula (IX-c) is of the following formula:

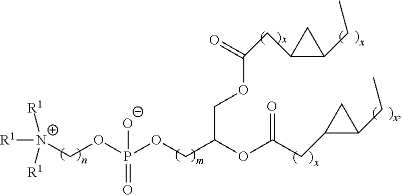

or a salt thereof.

In certain embodiments, the compound of Formula (H IX-c) is the following:

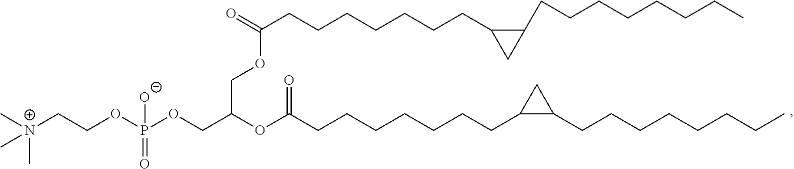 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

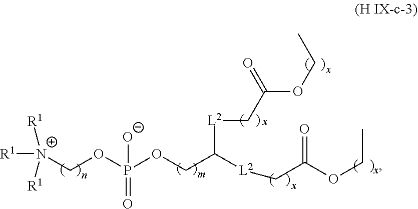

or a salt thereof.

In certain embodiments, the compound of Formula (H IX-c) is of the following formulae:

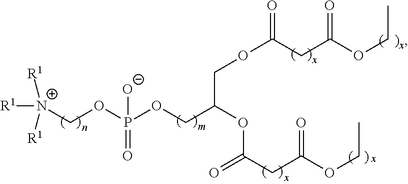

or a salt thereof.

In certain embodiments, the compound of Formula (H IX-c) is the following:

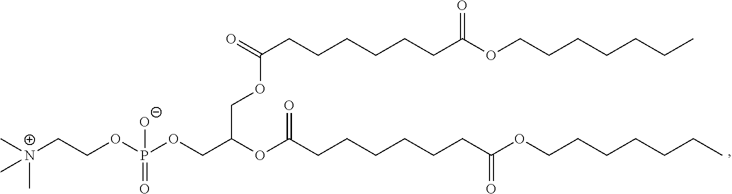 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

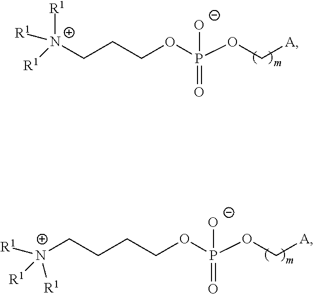

or a salt thereof.

In certain embodiments, a compound of Formula (H IX) is one of the following:

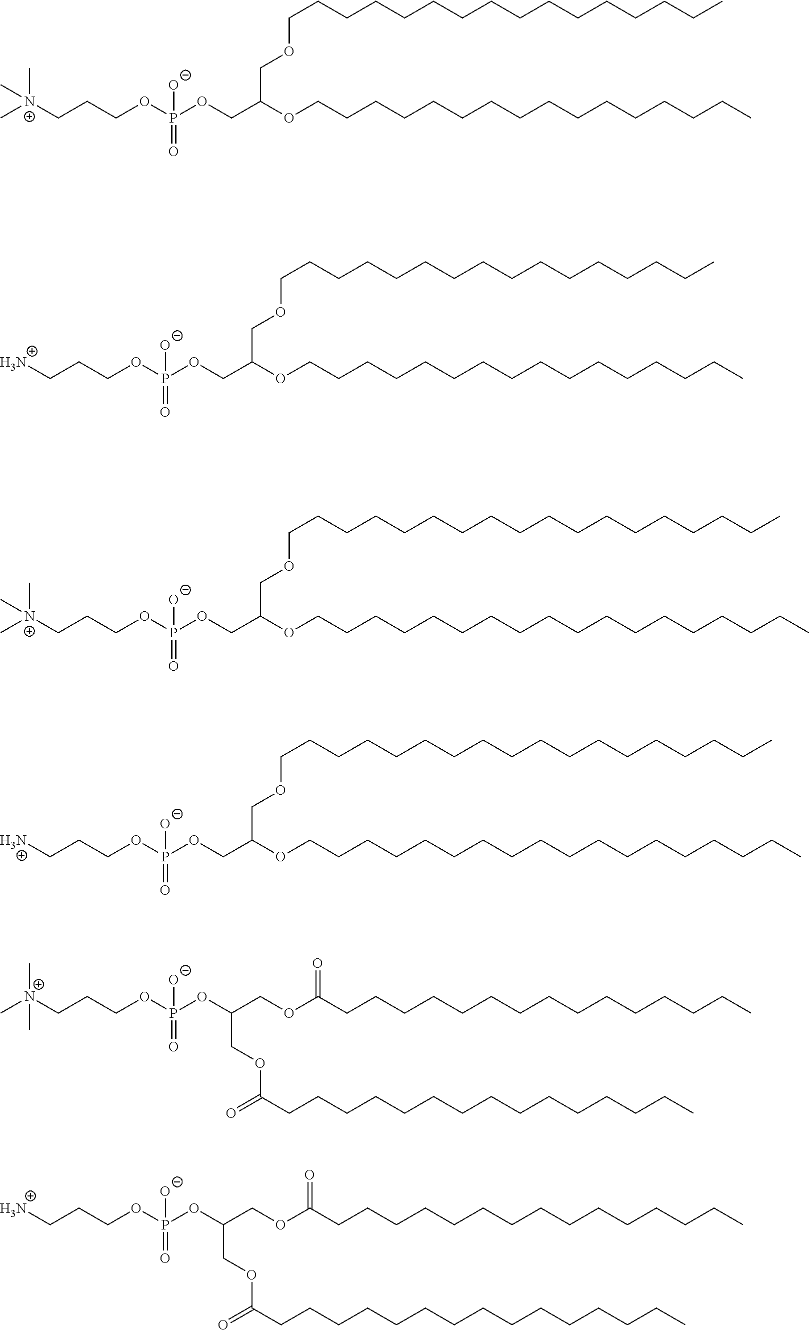

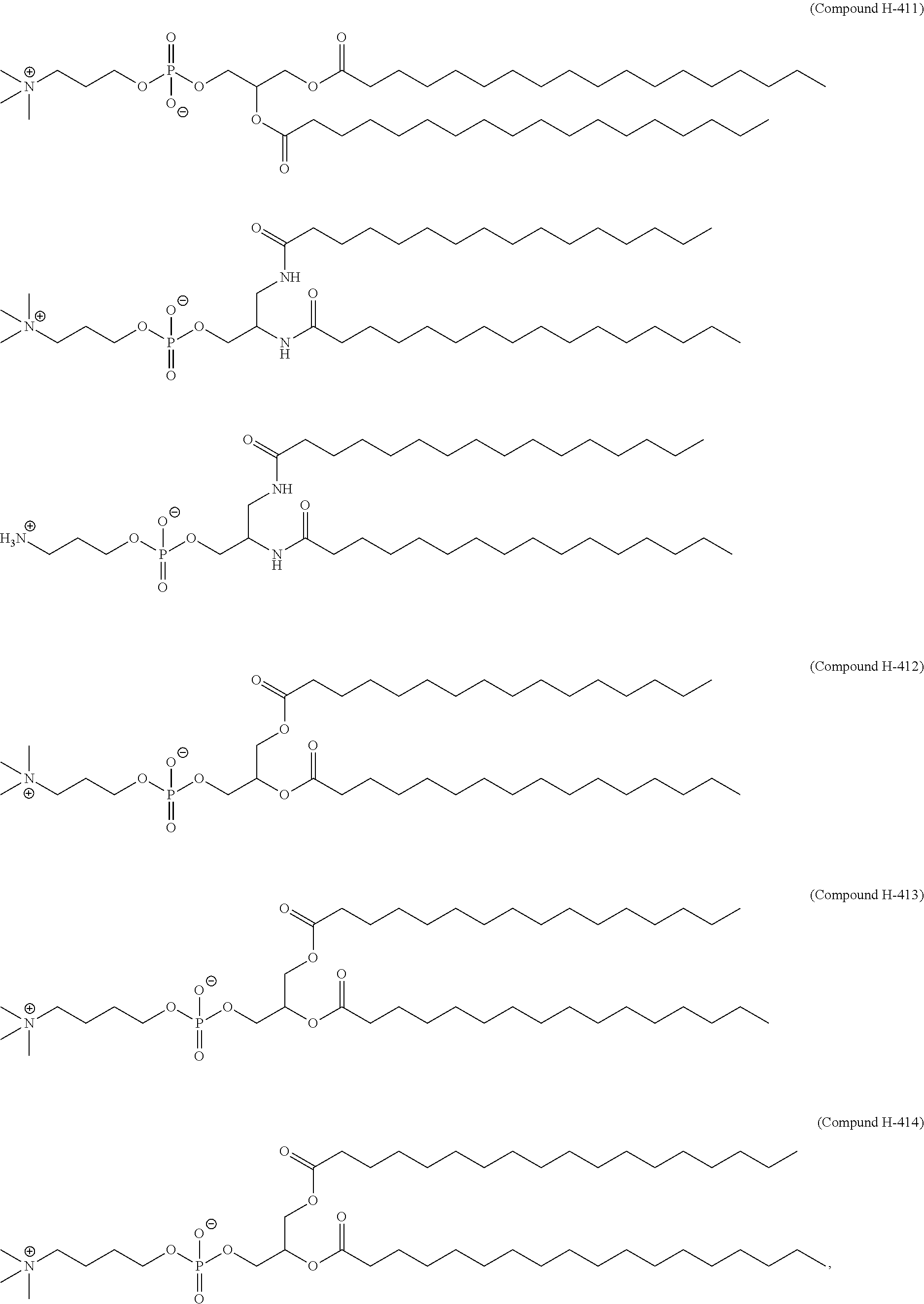

or salts thereof.

In certain embodiments, an alternative lipid is used in place of a phospholipid of the invention. Non-limiting examples of such alternative lipids include the following:

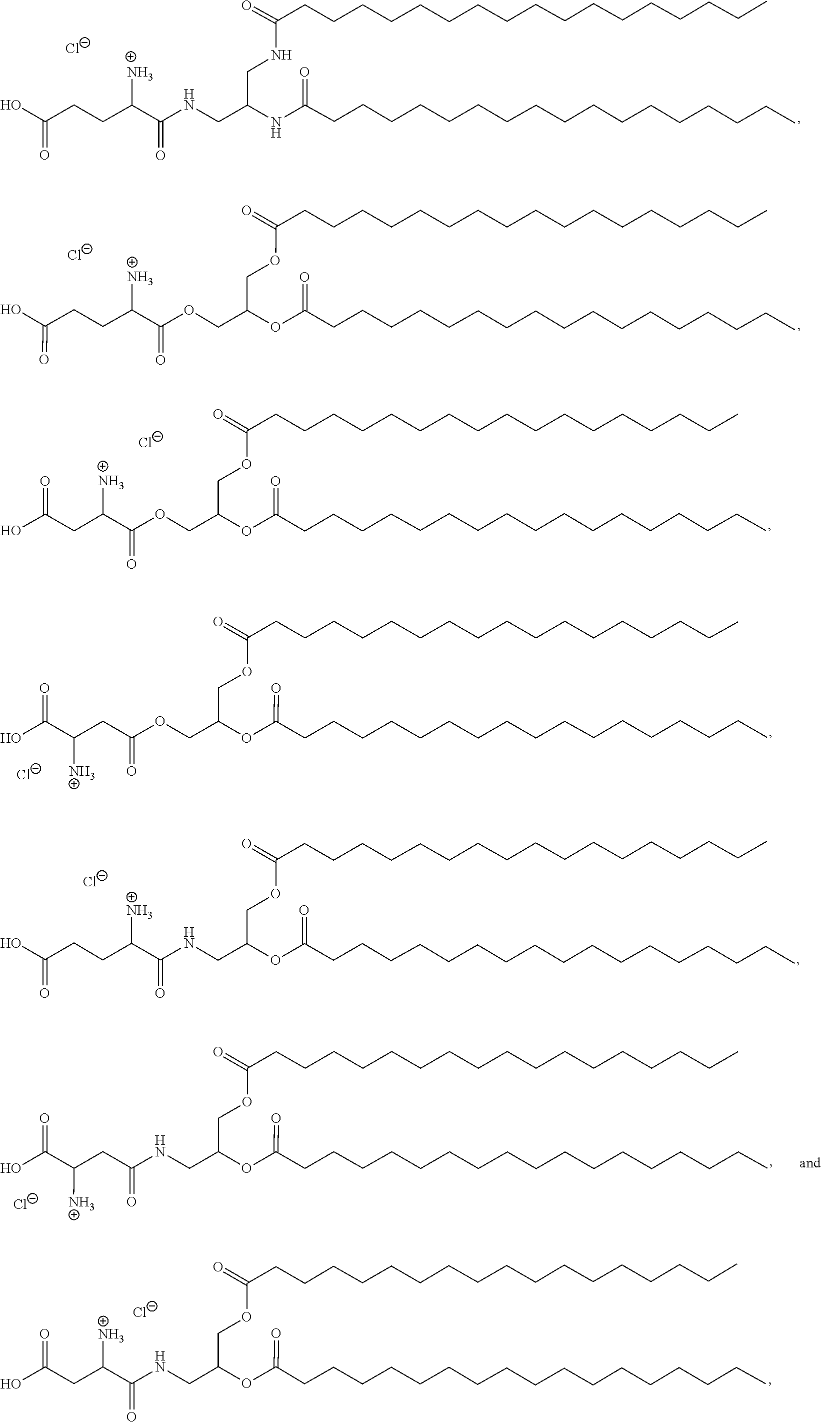

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R)—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O), —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N}—, —OS(O)₂N(R^{N})—, or —N(R^{N})S(O)₂O—.

In certain embodiments, the compound of Formula (H I-a) is of Formula (H I-c):

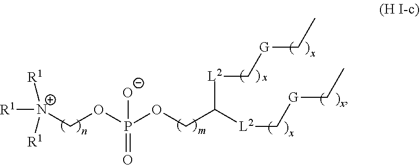

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N}), —NR^{N}C(O)—, —NR^{N}C(O)N(R^{N})—, —C(O)O—, —OC(O)—, —OC(O)O—, —OC(O)N(R^{N})—, —NR^{N}C(O)—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —N^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or N(R^{N})S(O)₂O—. Each possibility represents a separate embodiment of the present invention.

In certain embodiments, the compound of Formula (H I-c) is of Formula (H I-c-1):

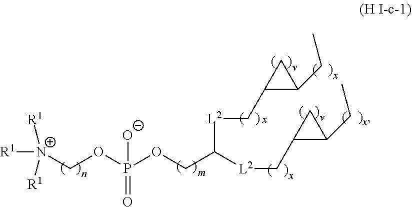

or salt thereof, wherein:

each instance of v is independently 1, 2, or 3.

In certain embodiments, the compound of Formula (H I-c) is of Formula (H I-c-2):

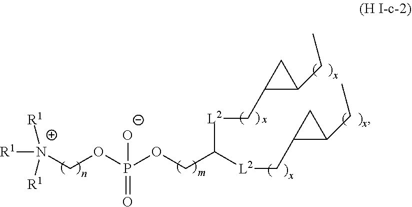

or a salt thereof.

In certain embodiments, the compound of Formula (I-c) is of the following formula:

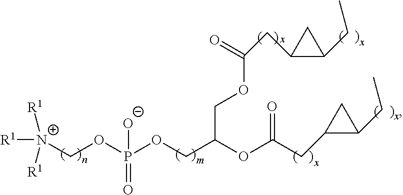

or a salt thereof.

In certain embodiments, the compound of Formula (H I-c) is the following:

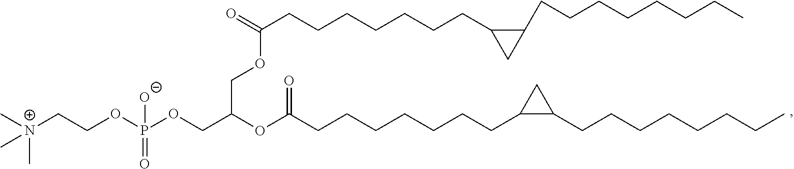 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

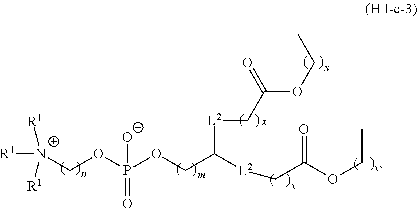

or a salt thereof.

In certain embodiments, the compound of Formula (H I-c) is of the following formulae:

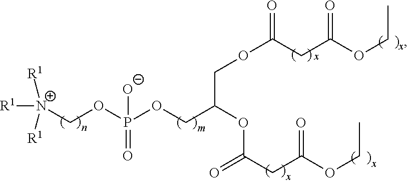

or a salt thereof.

In certain embodiments, the compound of Formula (H I-c) is the following:

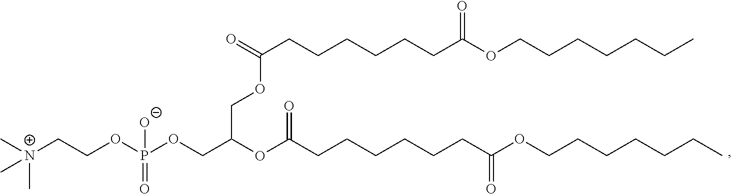 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

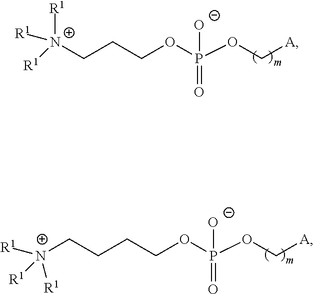

or a salt thereof.

In certain embodiments, a compound of Formula (H I) is one of the following:

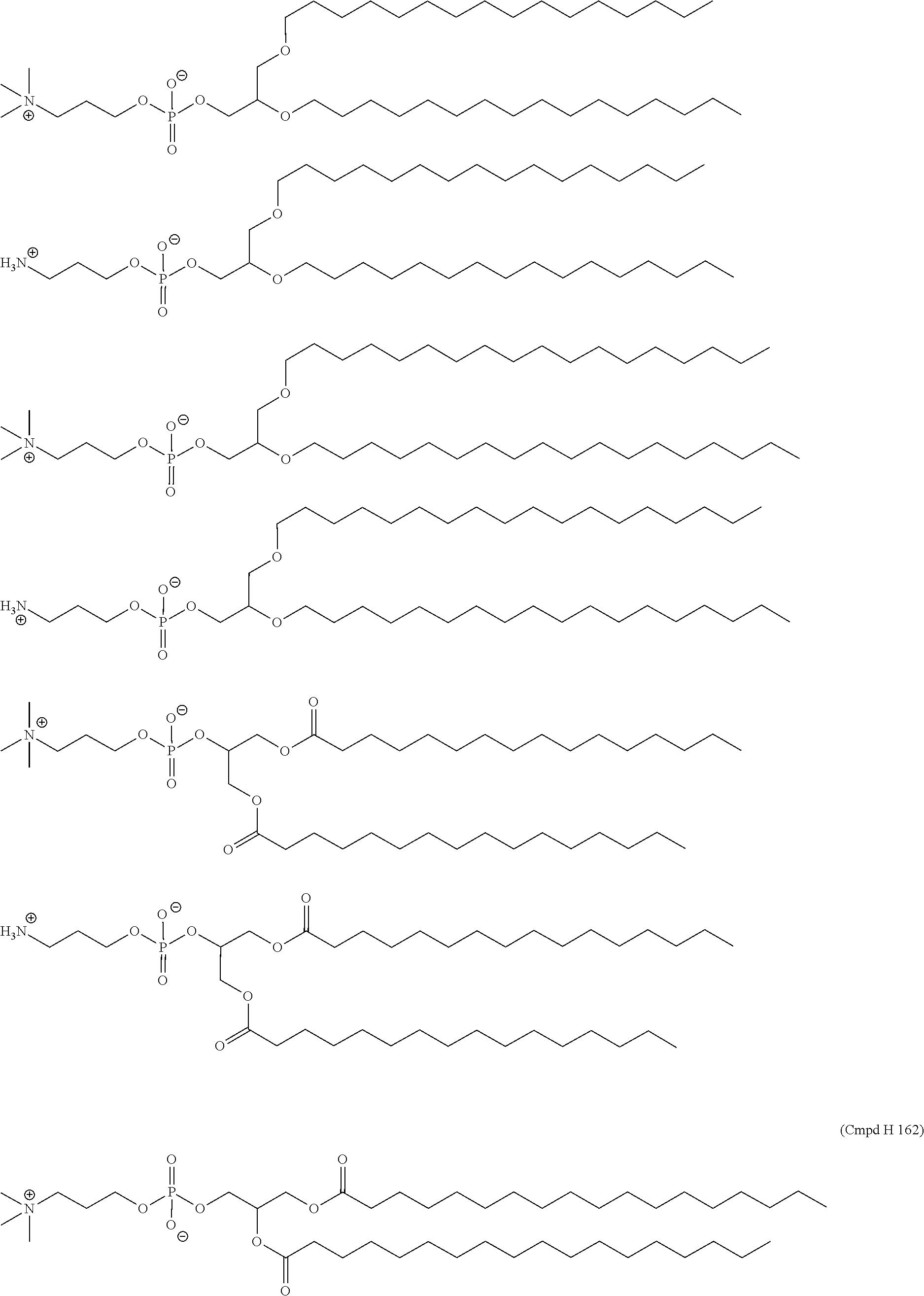

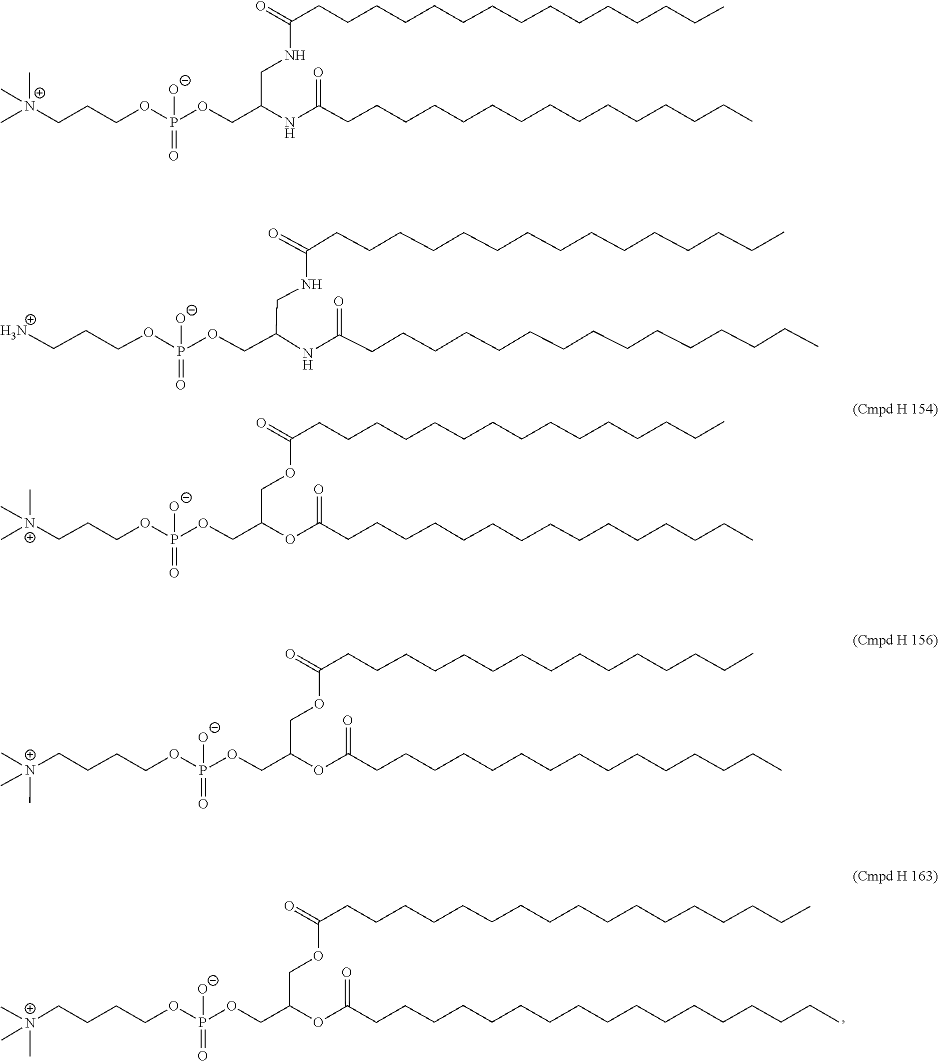

or salts thereof.

Numerous LNP formulations having phospholipids other than DSPC were prepared and tested for activity, as demonstrated in the examples below.

Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.
(i) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

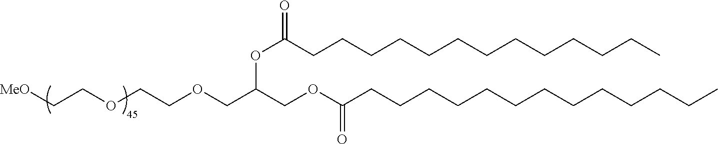

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.
In some embodiments, the PEG lipid is a compound of Formula (PI):

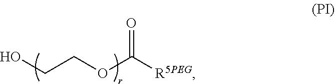

or a salt or isomer thereof, wherein:
r is an integer between 1 and 100; R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N}), —N(R^{N})S(O)₂N(R^{N})—, —OS(O)₂N(R^{N}), or N(R^{N})S(O)₂O—; and
each instance of R^{N} is independently hydrogen, C₁₋₆ alkyl, or a nitrogen protecting group.
   For example, R^{5PEG} is C₁₇ alkyl. For example, the PEG lipid is a compound of Formula (PI-a):

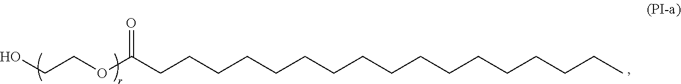

or a salt or isomer thereof, wherein r is an integer between 1 and 100.
For example, the PEG lipid is a compound of the following formula:

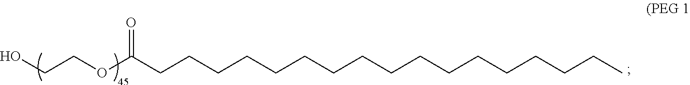

also referred to as Compound 428 below),
   or a salt or isomer thereof.
   The PEG lipid may be a compound of Formula (PII):

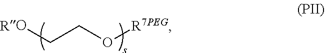

or a salt or isomer thereof, wherein:
s is an integer between 1 and 100;
R″ is a hydrogen, C₁₋₁₀ alkyl, or an oxygen protecting group;
   R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and
   each instance of R^{N} is independently hydrogen, C₁₋₆ alkyl, or a nitrogen protecting group. In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.
      In some embodiments, R″ is methyl.
      In some embodiments, the PEG lipid is a compound of Formula (PII-a):

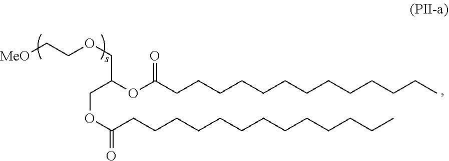

or a salt or isomer thereof, wherein s is an integer between 1 and 100.
For example, the PEG lipid is a compound of the following formula:

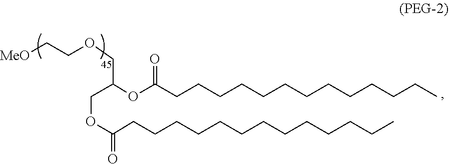

or a salt or isomer thereof.

In certain embodiments, a PEG lipid useful in the present invention is a compound of Formula (PIII). Provided herein are compounds of Formula (PIII):

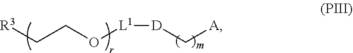

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

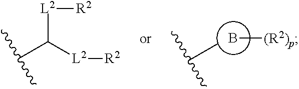

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

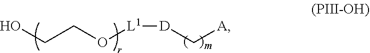

or a salt thereof.

In certain embodiments, D is a moiety obtained by click chemistry (e.g., triazole). In certain embodiments, the compound of Formula (PIII) is of Formula (PIII-a-1) or (PIII-a-2):

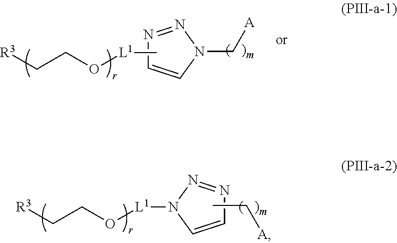

or a salt thereof.

In certain embodiments, the compound of Formula (PIII) is of one of the following formulae:

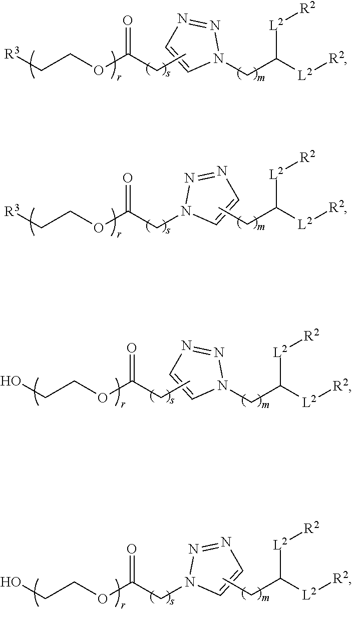

or a salt thereof, wherein
s is 0, 1, 2, 3, 4, 5, 6, 7, 8, 9, or 10.

In certain embodiments, the compound of Formula (PIII) is of one of the following formulae:

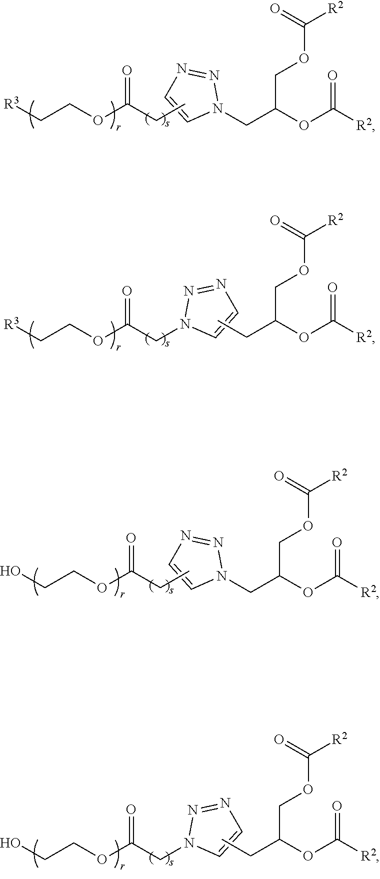

or a salt thereof.

In certain embodiments, a compound of Formula (PIII) is of one of the following formulae:

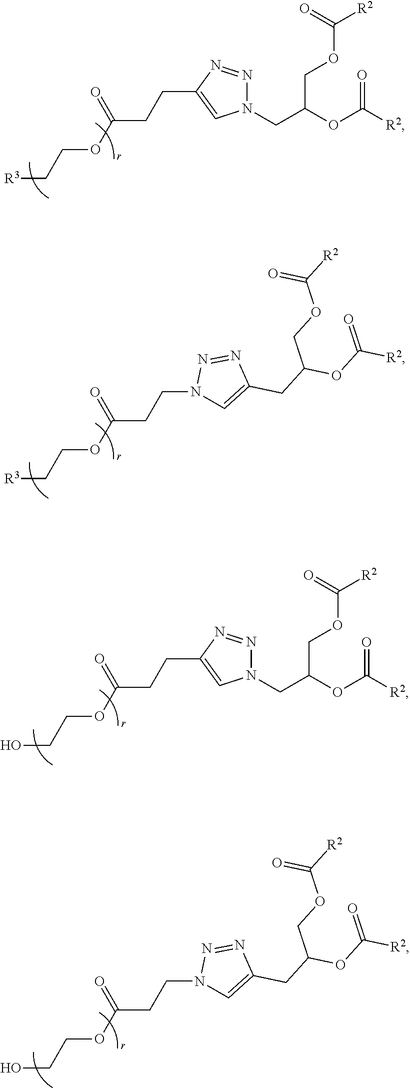

or a salt thereof.

In certain embodiments, a compound of Formula (PIII) is of one of the following formulae:

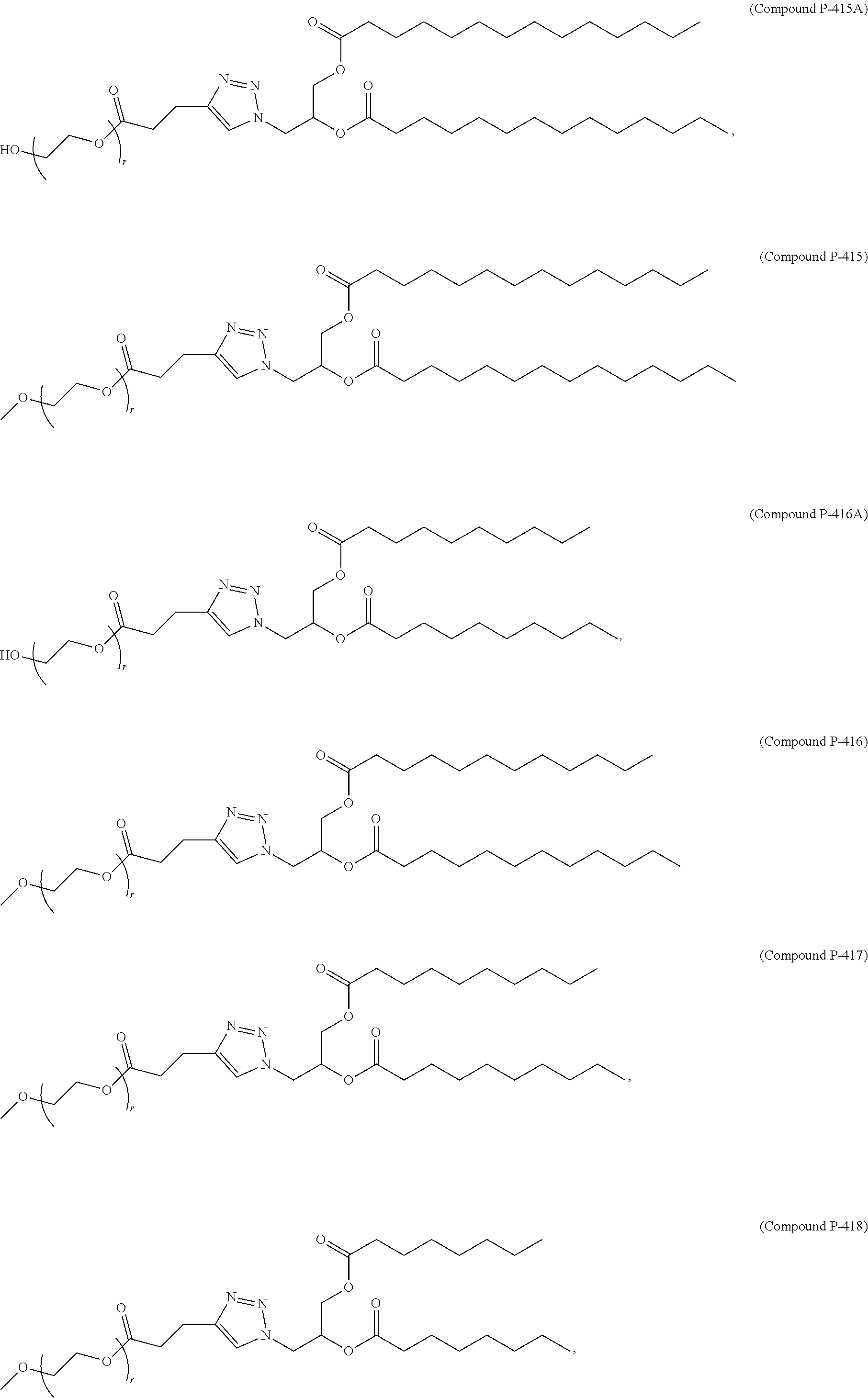

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

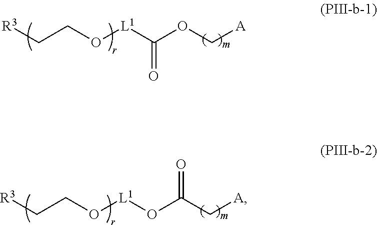

or a salt thereof.

In certain embodiments, a compound of Formula (PIII) is of Formula (PIII-b-1-OH) or (PIII-b-2-OH):

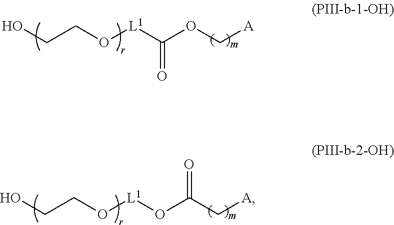

or a salt thereof.

In certain embodiments, the compound of Formula (PIII) is of one of the following formulae:

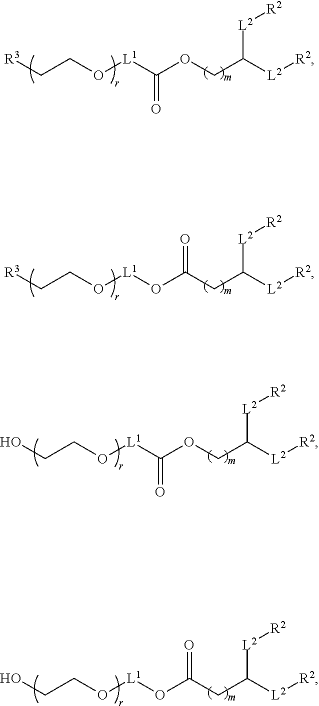

or a salt thereof.

In certain embodiments, a compound of Formula (PIII) is of one of the following formulae:

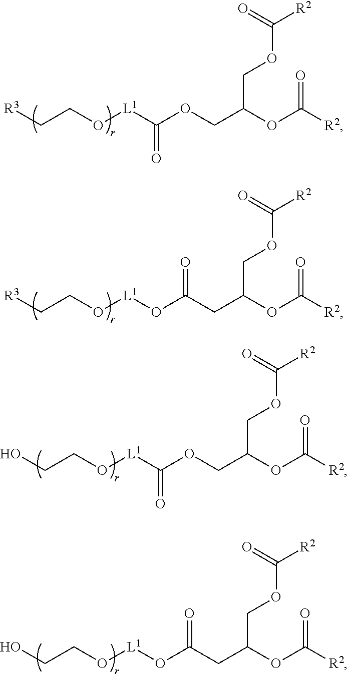

or a salt thereof.

In certain embodiments, a compound of Formula (PIII) is of one of the following formulae:

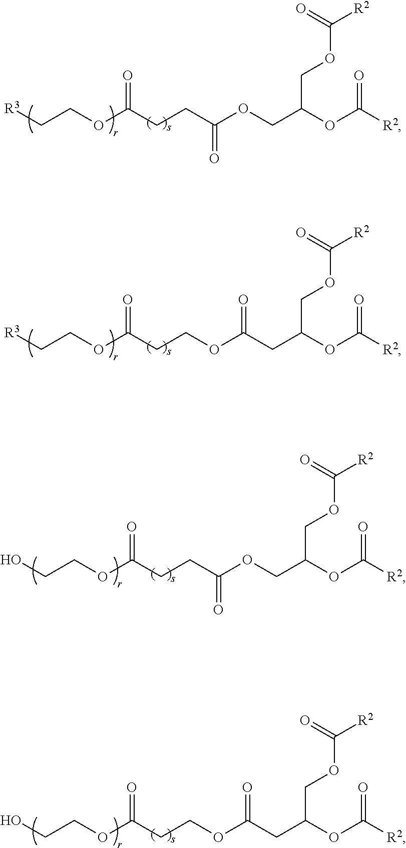

or a salt thereof.

In certain embodiments, a compound of Formula (PIII) is of one of the following formulae:

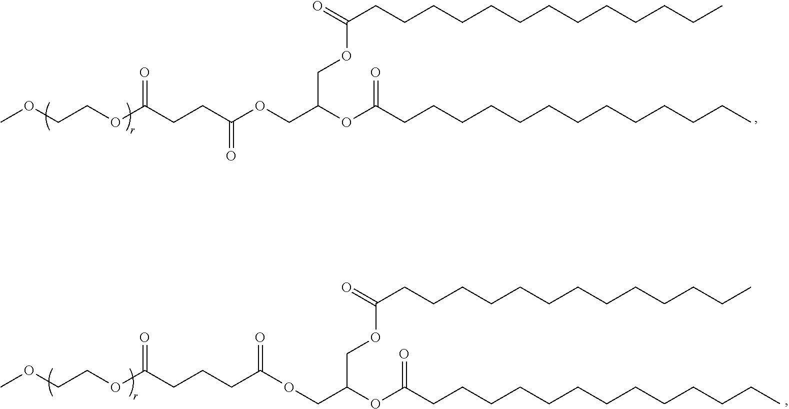

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

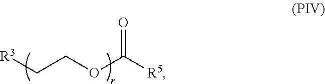

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PIV is of Formula (PIV-OH):

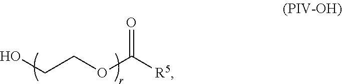

or a salt thereof. In some embodiments, r is 40-50. In some embodiments, r is 45.

In certain embodiments, a compound of Formula (PIV) is of one of the following formulae:

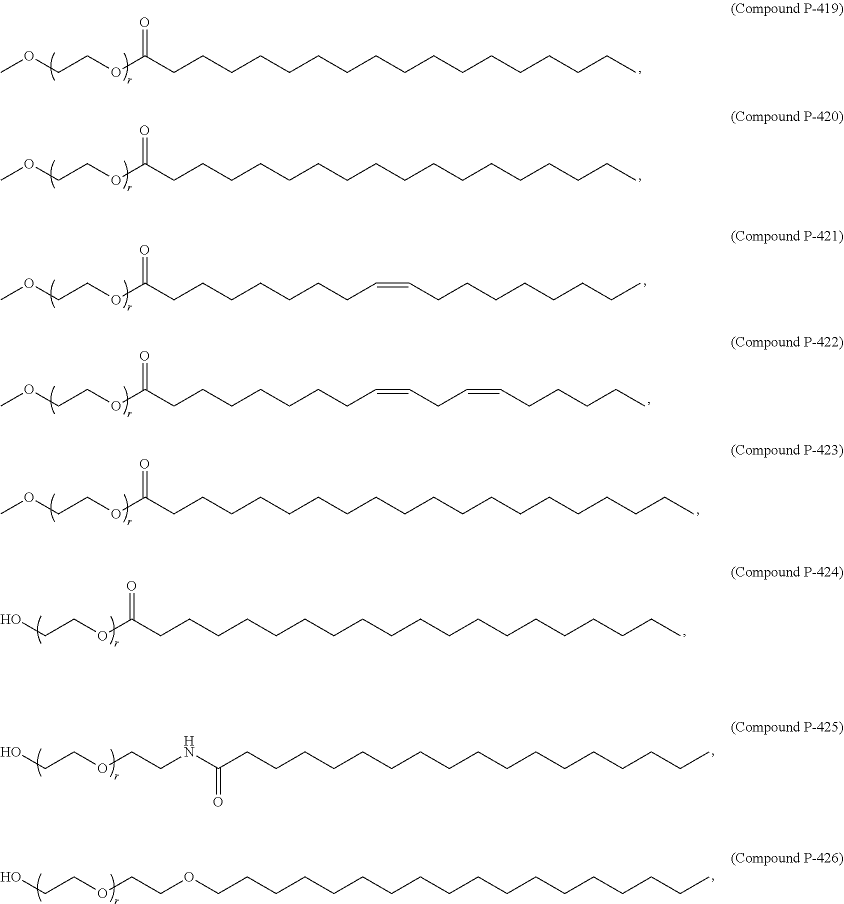

or a salt thereof. In some embodiments, r is 40-50. In some embodiments, r is 45.

In yet other embodiments the compound of Formula (PIV) is:

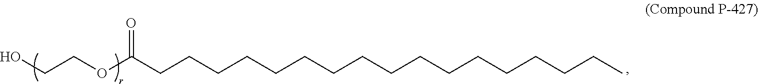

or a salt thereof.

In one embodiment, the compound of Formula (PIV) is

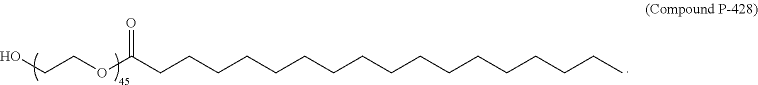

In one aspect, provided herein are lipid nanoparticles (LNPs) comprising PEG lipids of Formula (PV):

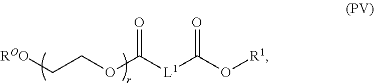

or pharmaceutically acceptable salts thereof, wherein:
L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;
R¹ is optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group; and
r is an integer from 2 to 100, inclusive.

In certain embodiments, the PEG lipid of Formula (PV) is of the following formula:

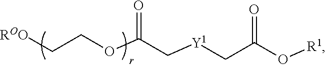

or a pharmaceutically acceptable salt thereof, wherein:
Y¹ is a bond, —CR₂—, —O—, —NR^{N}—, or —S—;
each instance of R is independently hydrogen, halogen, or optionally substituted alkyl; and
R^{N} is hydrogen, optionally substituted alkyl, optionally substituted acyl, or a nitrogen protecting group.

In certain embodiments, the PEG lipid of Formula (PV) is of one of the following formulae:

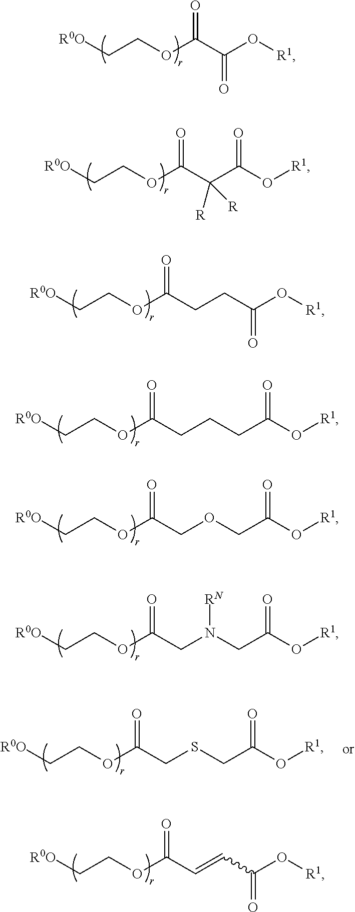

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

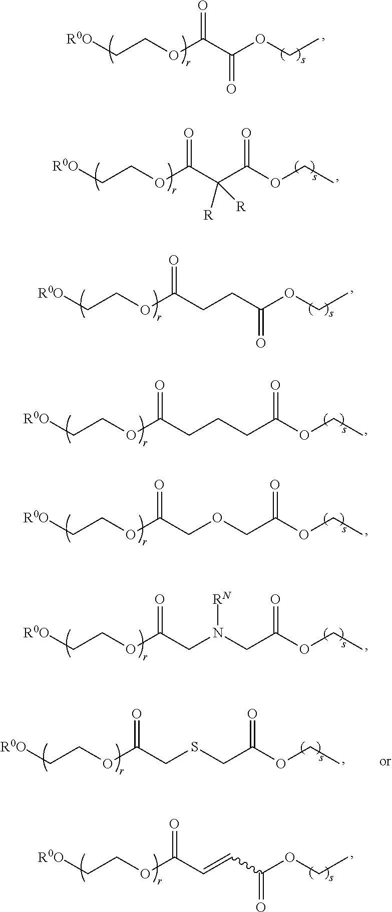

or a pharmaceutically acceptable salt thereof, wherein:

s is an integer from 5-25, inclusive.

In certain embodiments, the PEG lipid of Formula (PV) is of one of the following formulae:

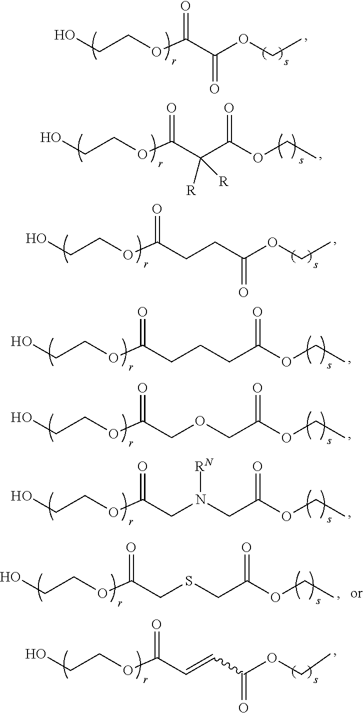

or a pharmaceutically acceptable salt thereof.

In certain embodiments, the PEG lipid of Formula (PV) is selected from the group consisting of:

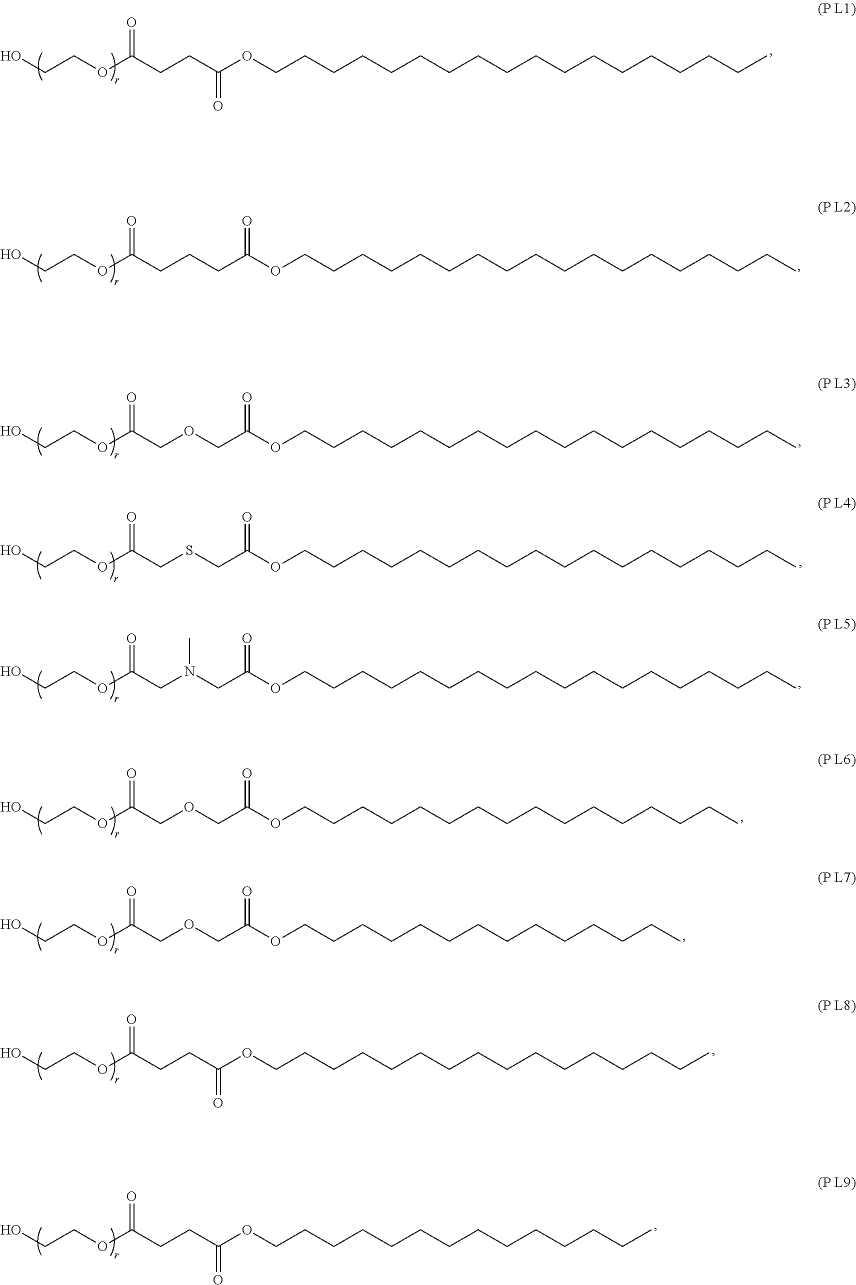

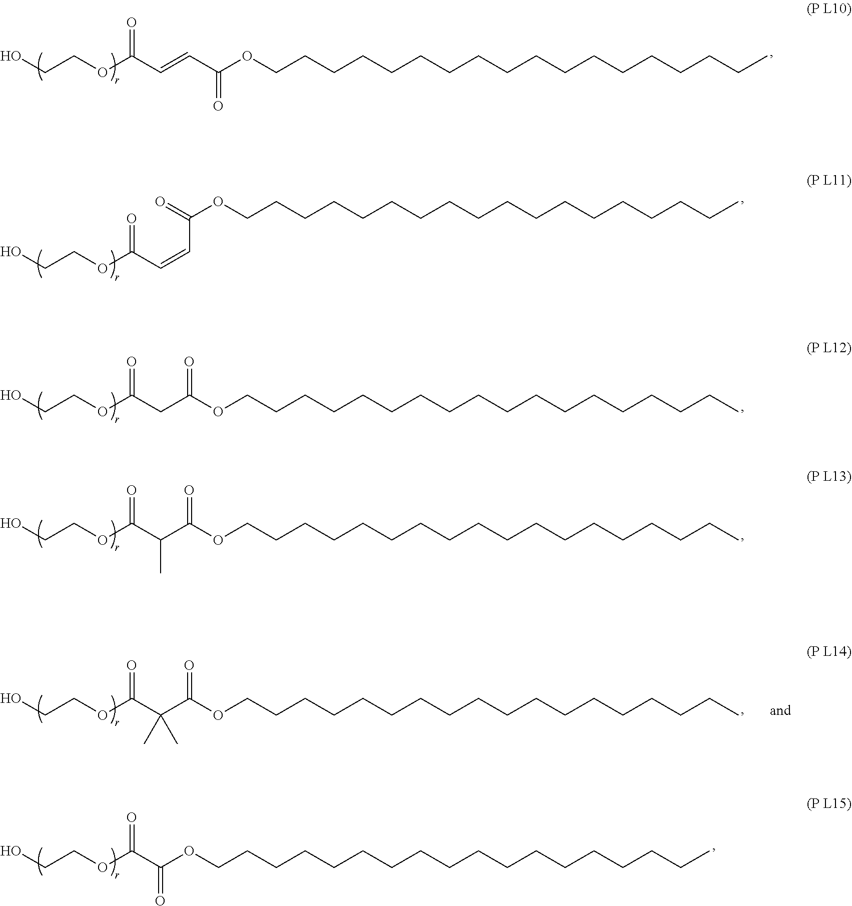

and pharmaceutically acceptable salts thereof.

In another aspect, provided herein are lipid nanoparticles (LNPs) comprising PEG lipids of Formula (PVI):

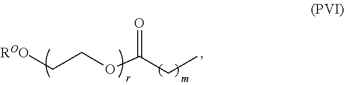

or pharmaceutically acceptable salts thereof, wherein:
R^{O} is hydrogen, optionally substituted alkyl, optionally substituted acyl, or an oxygen protecting group;
r is an integer from 2 to 100, inclusive; and
m is an integer from 5-15, inclusive, or an integer from 19-30, inclusive.

In certain embodiments, the PEG lipid of Formula (PVI) is of one of the following formulae:

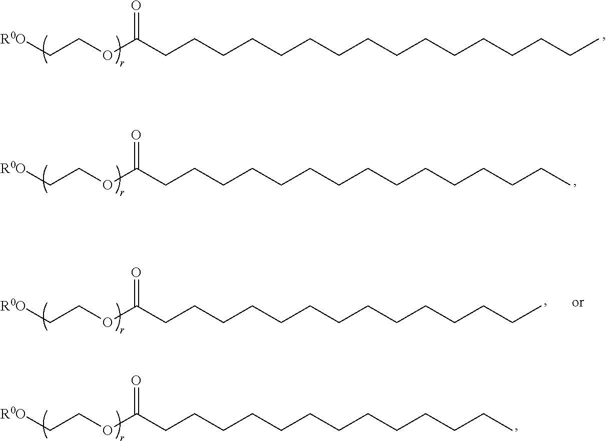

or a pharmaceutically acceptable salt thereof.

In certain embodiments, the PEG lipid of Formula (PVI) is of one of the following formulae:

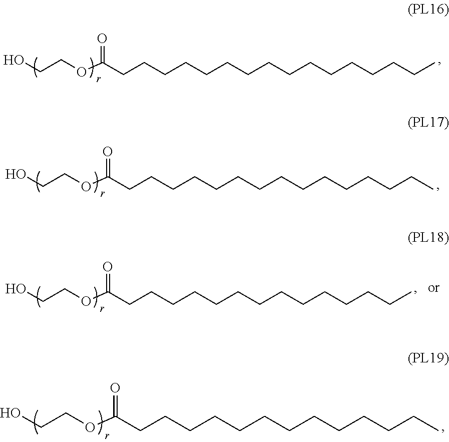

or a pharmaceutically acceptable salt thereof.

In another aspect, provided herein are lipid nanoparticles (LNPs) comprising PEG lipids of Formula (PVII):

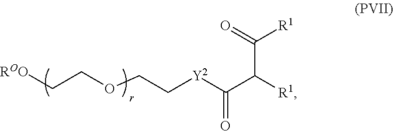

or pharmaceutically acceptable salts thereof, wherein:
Y² is —O—, —NR^{N}—, or —S—
each instance of R¹ is independently optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group;
R^{N} is hydrogen, optionally substituted alkyl, optionally substituted acyl, or a nitrogen protecting group; and
r is an integer from 2 to 100, inclusive.

In certain embodiments, the PEG lipid of Formula (PVII) is of one of the following formulae:

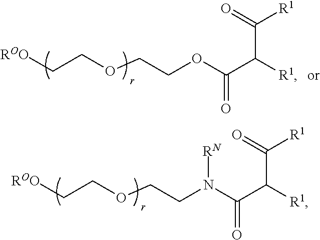

or a pharmaceutically acceptable salt thereof.

In certain embodiments, the PEG lipid of Formula (PVII) is of one of the following formulae:

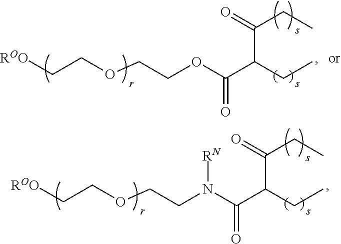

or a pharmaceutically acceptable salt thereof, wherein:

each instance of s is independently an integer from 5-25, inclusive.

In certain embodiments, the PEG lipid of Formula (PVII) is of one of the following formulae:

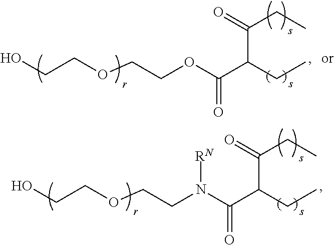

or a pharmaceutically acceptable salt thereof.

In certain embodiments, the PEG lipid of Formula (PVII) is selected from the group consisting of:

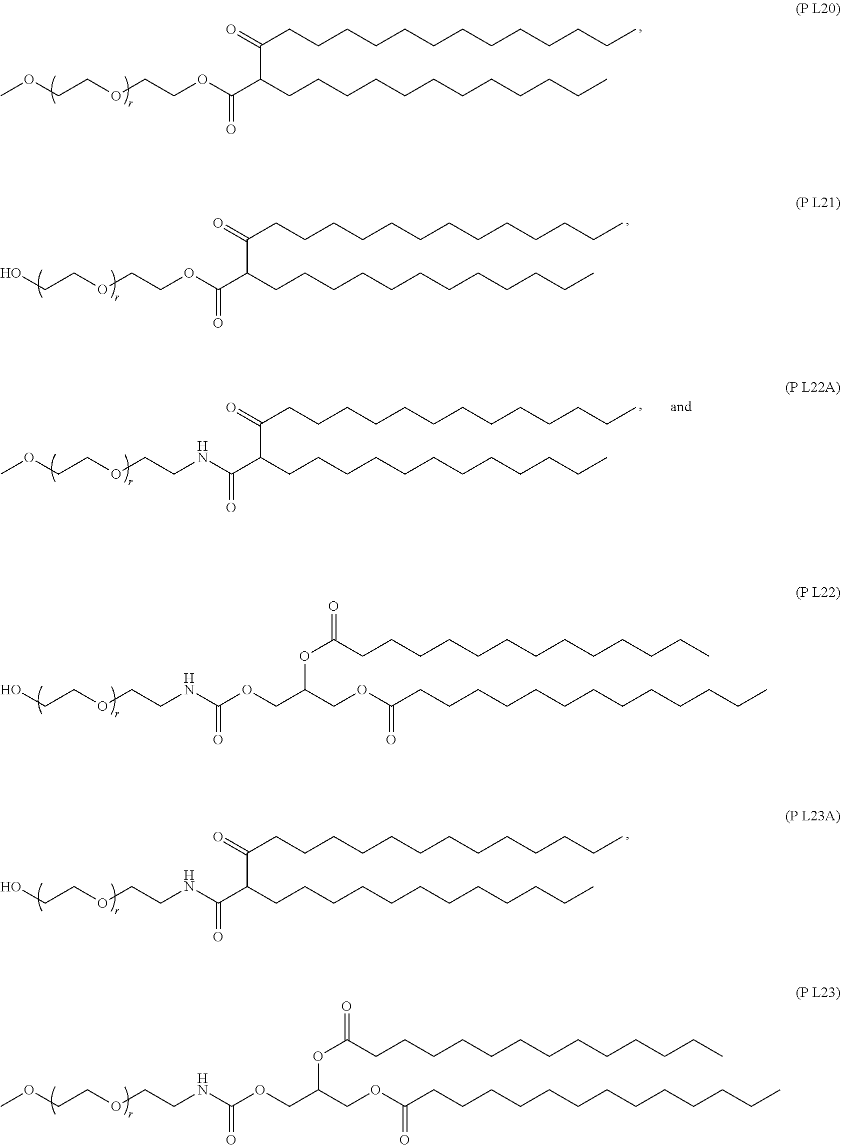

and pharmaceutically acceptable salts thereof.

In another aspect, provided herein are lipid nanoparticles (LNPs) comprising PEG lipids of Formula (PVIH):

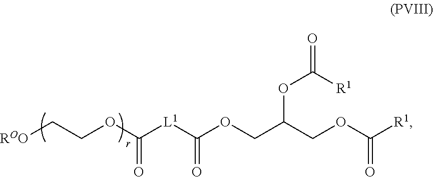

or pharmaceutically acceptable salts thereof, wherein:
   L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;
   each instance of R¹ is independently optionally substituted C₅₋₃₀ alkyl, optionally substituted C₃₋₃₀ alkenyl, or optionally substituted C₅₋₃₀ alkynyl;
   R^{O} is hydrogen, optionally substituted alkyl, optionally substituted acyl, or an oxygen protecting group;
   r is an integer from 2 to 100, inclusive;
   provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

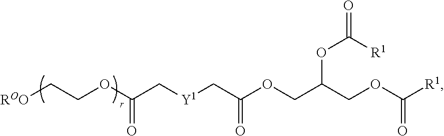

or a pharmaceutically acceptable salt thereof, wherein:
   Y¹ is a bond, —CR₂—, —O—, —NR^{N}—, or —S—;
   each instance of R is independently hydrogen, halogen, or optionally substituted alkyl;
   R^{N} is hydrogen, optionally substituted alkyl, optionally substituted acyl, or a nitrogen protecting group;
   provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

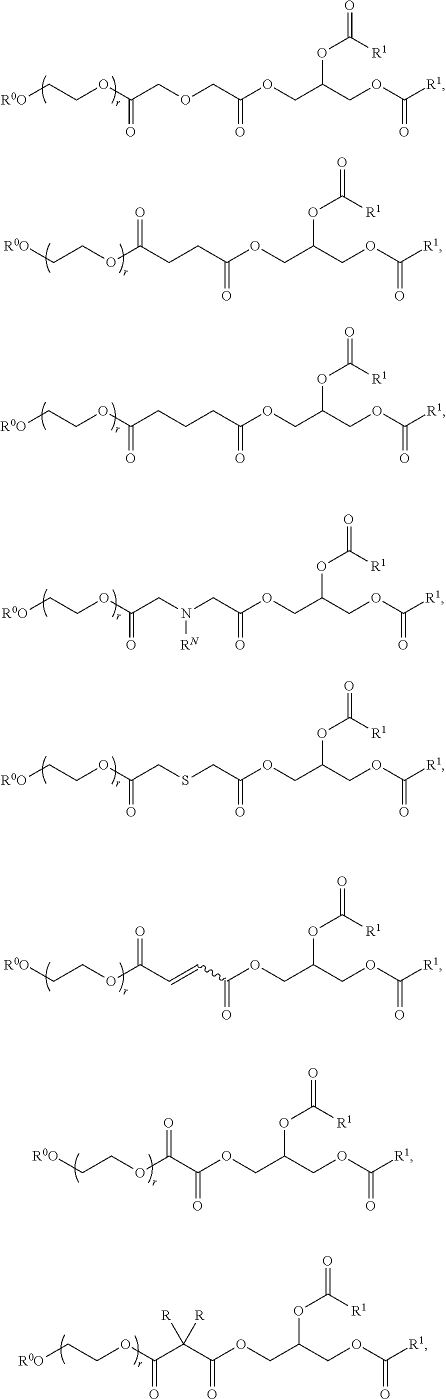

or a pharmaceutically acceptable salt thereof, wherein:
   each instance of R is independently hydrogen, halogen, or optionally substituted alkyl.

In certain embodiments, the PEG lipid of Formula (PVIII) is of one of the following formulae:

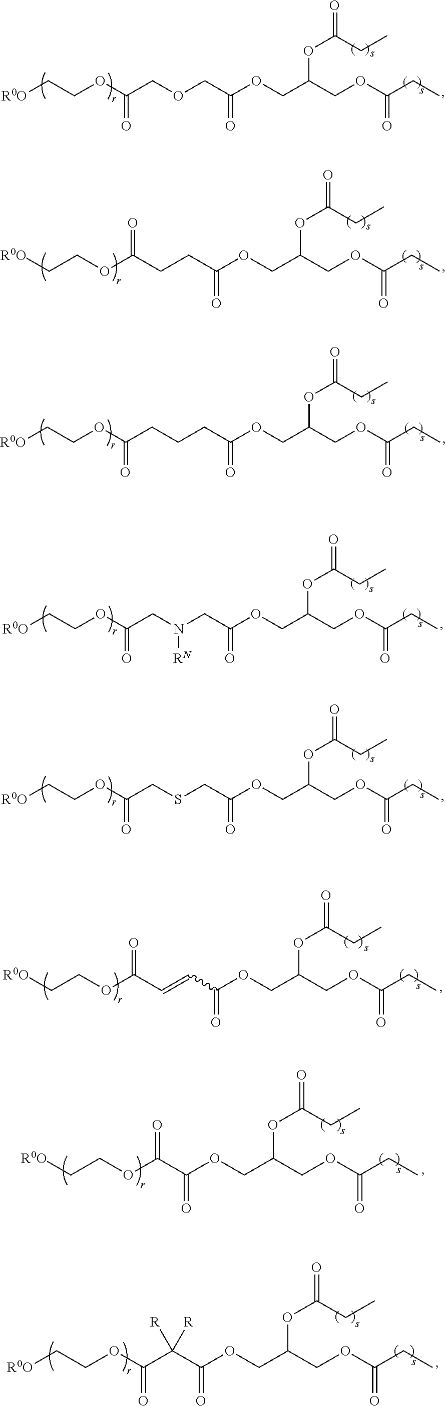

or a pharmaceutically acceptable salt thereof, wherein:
   each instance of R is independently hydrogen, halogen, or optionally substituted alkyl; and
   each s is independently an integer from 5-25, inclusive.

In certain embodiments, the PEG lipid of Formula (PVIII) is of one of the following formulae:

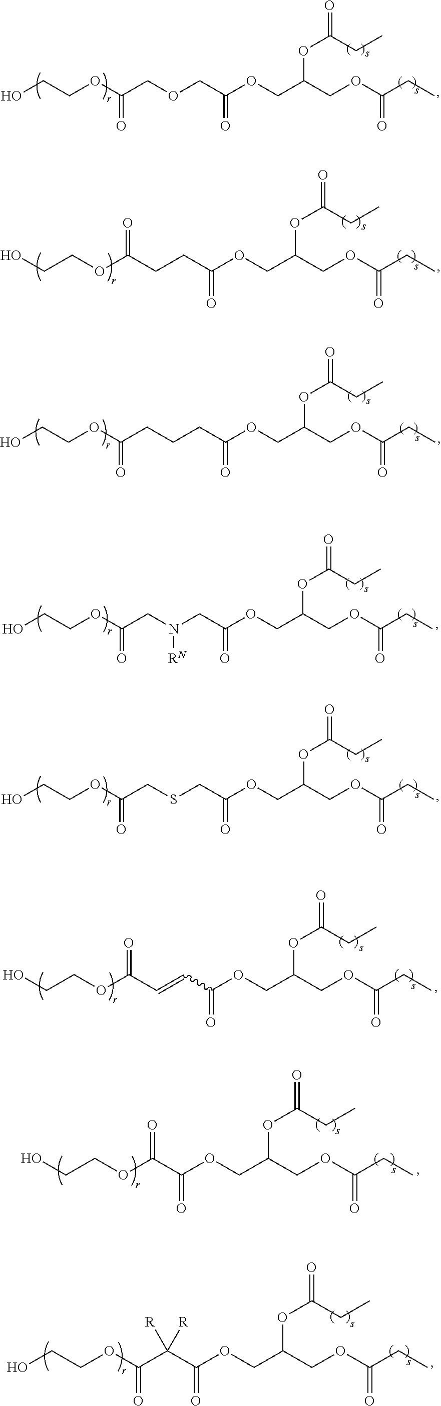

or a pharmaceutically acceptable salt thereof.

In certain embodiments, the PEG lipid of Formula (PVIII) is selected from the group consisting of:

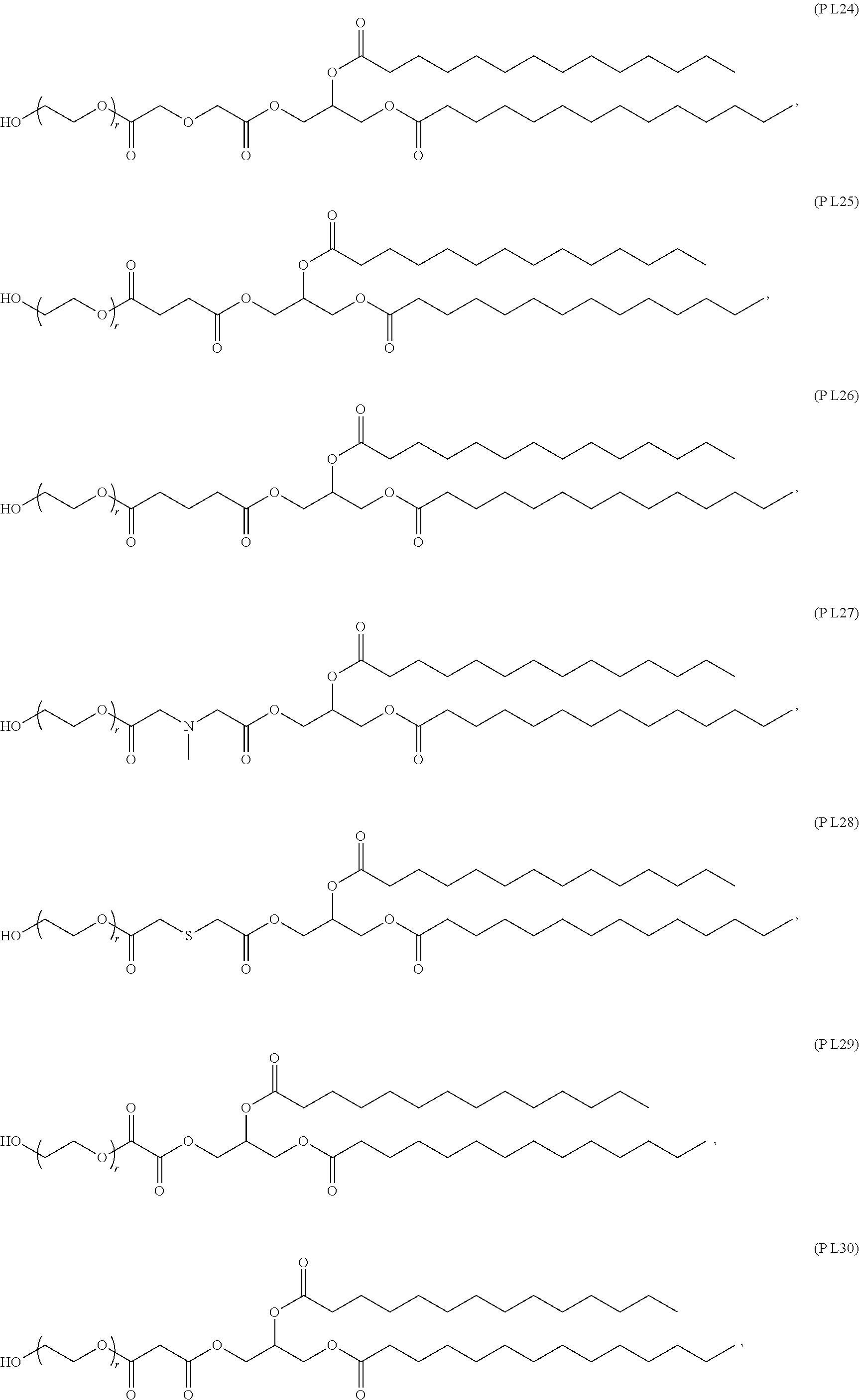

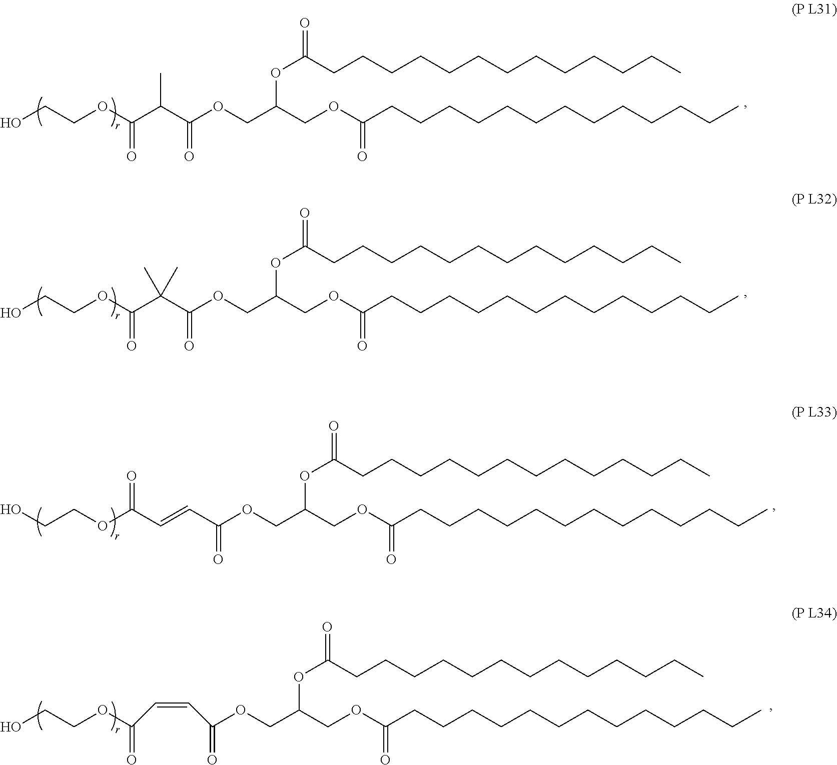

and pharmaceutically acceptable salts thereof.

In any of the foregoing or related aspects, a PEG lipid of the invention is featured wherein r is 40-50.

The LNPS provided herein, in certain embodiments, exhibit increased PEG shedding compared to existing LNP formulation comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids. In certain embodiments, the PEG lipids are present in a molar ratio of approximately 3% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 3 mol %. In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

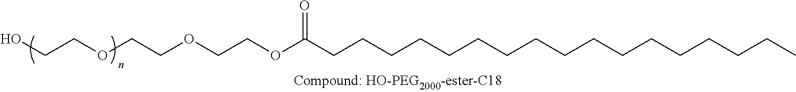

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated and backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, a target cell delivery lipid of the pharmaceutical compositions disclosed herein does not comprise a PEG-lipid.

In one embodiment, a target cell target cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, a target cell target cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of: Cmpd 428, PL16, PL17, PL 18, PL19, PL 1, and PL 2.

### Exemplary Additional LNP Components

### Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer.

For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10−4 M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

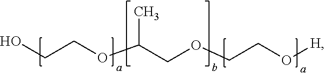

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof. For example, the polyethylene glycol ether is a compound of Formula (VIII):

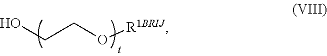

or a salt or isomer thereof, wherein:
t is an integer between 1 and 100;
R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}CS—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and
each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula VIII-a):

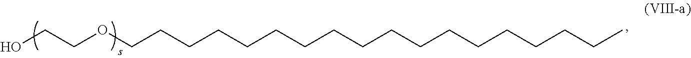

or a salt or isomer thereof.

In some embodiments, R^{1BRIJ} is Cis alkenyl. For example, the polyethylene glycol ether is a compound of Formula (VIII-b):

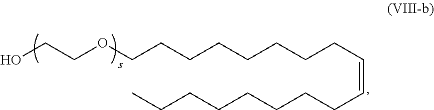

or a salt or isomer thereof.

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol. Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin p4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of an LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, M D, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER@ 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Methods of Using the LNP Compositions

In an aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, in the treatment and/or prophylaxis of a disease associated with an aberrant T regulatory cell function in a subject.

In a related aspect, provided herein is a method of treating and/or prophylaxis of a disease associated with an aberrant T regulatory cell function in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

In another aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for inhibiting an immune response in a subject.

In a related aspect, provided herein is method of inhibiting an immune response in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

In an aspect, the disclosure provides a composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for stimulating T regulatory cells in a subject.

In a related aspect, provided herein is a method of stimulating T regulatory cells in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

In another aspect, the disclosure provides a composition comprising a lipid nanoparticle (LNP) comprising a first polynucleotide encoding an IL-2 molecule and a second polynucleotide encoding a GM-CSF molecule for use, in the treatment and/or prophylaxis of a disease associated with an aberrant T regulatory cell function in a subject.

In a related aspect, provided herein is a method of treating and/or prophylaxis of a disease associated with an aberrant T regulatory cell function in a subject, comprising administering to the subject an effective amount of a lipid nanoparticle (LNP) comprising a first polynucleotide encoding an IL-2 molecule and a second polynucleotide encoding a GM-CSF molecule.

In an embodiment, prior to the administration of the LNP comprising the first polynucleotide encoding the IL-2 molecule and the second polynucleotide encoding the GM-CSF molecule, a different LNP comprising a third polynucleotide encoding a GM-CSF molecule is administered to the subject.

In an embodiment, the LNP comprising a third polynucleotide encoding the GM-CSF molecule does not comprise a polynucleotide encoding an IL-2 molecule.

In an embodiment, the second polynucleotide encoding GM-CSF and the third polynucleotide encoding GM-CSF comprise the same or substantially the same polynucleotide sequence.

In an embodiment, the different LNP comprising a third polynucleotide encoding a GM-CSF molecule is administered at least 1 day, 2 days, 3 days, 4 days, 5 days, 6 days, 7 days, 8 days, 9 days, or 10 days (e.g., 7 days), prior to the administration of the LNP comprising a first polynucleotide encoding an IL-2 molecule and a second polynucleotide encoding a GM-CSF molecule.

In an embodiment, the different LNP comprising a third polynucleotide encoding a GM-CSF molecule is administered at least 1 week, 2 weeks, 3 weeks, 4 weeks, 5 weeks, or 6 weeks prior to the administration of the LNP comprising a first polynucleotide encoding an IL-2 molecule and a second polynucleotide encoding a GM-CSF molecule.

In an embodiment, the LNP comprising the first polynucleotide encoding an IL-2 molecule and a second polynucleotide encoding a GM-CSF molecule, and the LNP comprising a third polynucleotide encoding a GM-CSF molecule are administered at a dose disclosed herein. In an embodiment, the dose, e.g., effective dose, of the GM-CSF molecule in the LNP comprising the third polynucleotide encoding GM-CSF is at least 10%, 20%, 30%, 40%, 50%, 60%, 70%, 80% or 95% lesser than the dose, e.g., effective dose, of the GM-CSF molecule in the LNP comprising the first and second polynucleotides. In an embodiment, the dose, e.g., effective dose, of the first polynucleotide encoding the IL-2 molecule in the lipid nanoparticle is at least 10%, 20%, 30%, 40%, 50%, 60%, 70%, 80% or 95% lesser than the dose of a naturally occurring, or recombinant IL-2, e.g., in an otherwise similar LNP.

In an aspect, the disclosure provides, a lipid nanoparticle comprising a polynucleotide encoding a molecule that stimulates T regulatory cells (e.g., an IL-2 molecule) for use, in the treatment and/or prophylaxis of a disease associated with an aberrant T regulatory cell function in a subject.

In another aspect, provided herein is a method of treating and/or preventing a disease associated with an aberrant T regulatory cell function in a subject, comprising administering to the subject an effective amount of a lipid nanoparticle comprising a polynucleotide encoding a molecule that stimulates T regulatory cells (e.g., an IL-2 molecule).

In an embodiment, the method or composition for use further comprises administration of a lipid nanoparticle comprising a polynucleotide encoding a GM-CSF molecule.

In an embodiment, the molecule that stimulates T regulatory cells comprises an IL-2 molecule, or a molecule that binds to a receptor present on T regulatory cells.

In yet another aspect, the disclosure provides a lipid nanoparticle (LNP) comprising a polynucleotide encoding a molecule that stimulates dendritic cells (e.g., a GM-CSF molecule) for use, in the treatment and/or prophylaxis of a disease associated with an aberrant T regulatory cell function in a subject.

In a related aspect, provided herein is a method of treating and/or preventing a disease associated with an aberrant T regulatory cell function in a subject, comprising administering to a subject an effective amount of a lipid nanoparticle comprising a polynucleotide encoding molecule that stimulates dendritic cells (e.g., a GM-CSF molecule).

In an embodiment, the method or composition for use further comprises administration of a lipid nanoparticle comprising a polynucleotide encoding an IL-2 molecule.

In an embodiment, the molecule that stimulates dendritic cells comprises a molecule that stimulates, e.g., increases, the expression and/or level of TNF alpha, IL-10, CCL-2 and/or nitric oxide in dendritic cells.

In an embodiment, the molecule that stimulates dendritic cells comprises a GM-CSF molecule, e.g., as described herein.

In an embodiment, the molecule that stimulates dendritic cells results in an increased level and/or activity of CD11b+ or CD11c+ dendritic cells.

In an embodiment, administration of the LNP comprising the polynucleotide encoding the GM-CSF molecule results in a modulation of dendritic cell activity and/or modulation of expression and/or activity of myeloid cells in a sample from the subject. In an embodiment, the sample has an increase in, e.g., increased number or proportion of, dendritic cells expressing CD11b and/or CD11c. In an embodiment, the increase in DCs expressing CD11c (CD11c+ DCs) is at least 1.2-20 fold (e.g., at least 1.2, 1.5, 2, 3, 4, 5, 6, 7, 8, 9, 10, 11, 12, 13, 14, 15, 16, 17, 18, 19, or 20 fold), e.g., as compared to an otherwise similar sample not contacted with the LNP comprising the GM-CSF molecule, or contacted with a different LNP.

In an embodiment, the sample has an increase in, e.g., increased number or proportion of, myeloid cells expressing CD11b, e.g., as compared to an otherwise similar sample not contacted with the LNP comprising the GM-CSF molecule, or contacted with a different LNP.

In an embodiment of any of the compositions or methods provided herein, one or more LNP compositions described herein is administered subcutaneously.

### Diseases and Disorders

In an embodiment of any of the methods of treatment or compositions for use disclosed herein, the subject has, or is identified as having, a disease or disorder associated with aberrant T cell function, e.g., aberrant T regulatory cell function. In an embodiment of any of the methods of prophylaxis of a disease or disorder or compositions for use disclosed herein, the subject has, is susceptible to, or is identified as having, the disease or disorder to which the method or composition is directed. In an embodiment, the disease is an autoimmune disease, or a disease with hyper-activated immune function. In an embodiment, an LNP disclosed herein is administered to the subject to treat or ameliorate a symptom of the disease or disorder. In an embodiment, an LNP disclosed herein is administered to a subject to inhibit an immune response in the subject.

In an embodiment, the autoimmune disease is chosen from: rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)), multiple sclerosis; autoimmune hepatitis (e.g., Type 1 or Type 2); primary biliary cholangitis; organ transplant associated rejection; myasthenia gravis; Parkinsons's Disease; Alzheimer's Disease; amyotrophic lateral sclerosis; psoriasis; or polymyositis (also known as dermatomyositis).

In an embodiment, the autoimmune disease is rheumatoid arthritis (RA).

In an embodiment, the autoimmune disease is graft versus host disease (GVHD) (e.g., acute GVHD or chronic GVHD).

In an embodiment, the autoimmune disease is diabetes, e.g., Type 1 diabetes.

In an embodiment, the autoimmune disease is inflammatory bowel disease (IBD). In an embodiment, IBD comprises colitis, ulcerative colitis or Crohn's disease.

In an embodiment, the autoimmune disease is lupus, e.g., systemic lupus erythematosus (SLE).

In an embodiment, the autoimmune disease is multiple sclerosis.

In an embodiment, the autoimmune disease is autoimmune hepatitis, e.g., Type 1 or Type 2.

In an embodiment, the autoimmune disease is primary biliary cholangitis.

In an embodiment, the autoimmune disease is organ transplant associated rejection. In an embodiment, an organ transplant associated rejection comprises renal allograft rejection; liver transplant rejection; bone marrow transplant rejection; or stem cell transplant rejection. In an embodiment, a stem cell transplant comprises a transplant of any one or all of the following types of cells: stem cells, cord blood stem cells, hematopoietic stem cells, embryonic stem cells, cells derived from or comprising mesenchymal stem cells, and/or induced stem cells (e.g., induced pluripotent stem cells). In an embodiment, the stem cell comprises a pluripotent stem cell.

In an embodiment, the autoimmune disease is myasthenia gravis.

In an embodiment, the autoimmune disease is Parkinson's disease.

In an embodiment, the autoimmune disease is Alzheimer's disease.

In an embodiment, the autoimmune disease is amyotrophic lateral sclerosis.

In an embodiment, the autoimmune disease is psoriasis.

In an embodiment, the autoimmune disease is polymyositis.

In an embodiment the subject is a mammal, e.g., a human.

### Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA In some aspects, the subject for the present methods or compositions has been treated with one or more standard of care therapies. In other aspects, the subject for the present methods or compositions has not been responsive to one or more standard of care therapies or anti-cancer therapies.

### Sequence Optimization and Methods Thereof

In some embodiments, a polynucleotide of the disclosure comprises a sequence-optimized nucleotide sequence encoding a polypeptide disclosed herein, e.g., IL-2 and/or GM-CSF. In some embodiments, the polynucleotide of the disclosure comprises an open reading frame (ORF) encoding an IL-2 polypeptide, wherein the ORF has been sequence optimized. In some embodiments, the polynucleotide of the disclosure comprises an open reading frame (ORF) encoding a GM-CSF polypeptide, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding an IL-2 polypeptide, a GM-CSF polypeptide, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine is present instead of uracil, and one would substitute T where U appears. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding an IL-2 polypeptide, or a GM-CSF polypeptide of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding an IL-2 polypeptide, or a GM-CSF polypeptide of the disclosure has a reduced number of uracil triplets (UUU) with respect to the wild-type nucleic acid sequence. In some embodiments, a uracil-modified sequence encoding an IL-2 polypeptide, or a GM-CSF polypeptide has a reduced number of uracil pairs (UU) with respect to the number of uracil pairs (UU) in the wild-type nucleic acid sequence. In some embodiments, a uracil-modified sequence encoding an IL-2 polypeptide, or a GM-CSF polypeptide of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding an IL-2 polypeptide or a GM-CSF polypeptide has a % UUwt between below 100%.

In some embodiments, the polynucleotide of the disclosure comprises a uracil-modified sequence encoding an IL-2 polypeptide, or a GM-CSF polypeptide disclosed herein. In some embodiments, the uracil-modified sequence encoding an IL-2 polypeptide, or a GM-CSF polypeptide comprises at least one chemically modified nucleobase, e.g., 5-methoxyuracil. In some embodiments, at least 95% of a nucleobase (e.g., uracil) in a uracil-modified sequence encoding an IL-2 polypeptide, or a GM-CSF polypeptide of the disclosure are modified nucleobases. In some embodiments, at least 95% of uracil in a uracil-modified sequence encoding an IL-2 polypeptide, or a GM-CSF polypeptide is 5-methoxyuracil. In some embodiments, the polynucleotide comprising a uracil-modified sequence further comprises a miRNA binding site, e.g., a miRNA binding site that binds to miR-122. In some embodiments, the polynucleotide comprising a uracil-modified sequence is formulated with a delivery agent, e.g., a compound having Formula (I), e.g., any of Compounds 1-147, or any of Compounds 1-232.

In some embodiments, a polynucleotide of the disclosure (e.g., a polynucleotide comprising a nucleotide sequence encoding an IL-2 polypeptide, or a GM-CSF polypeptide (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding an IL-2 polypeptide, or a GM-CSF polypeptide). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active IL-2 or GM-CSF polypeptide.

Additional and exemplary methods of sequence optimization are disclosed in International PCT application WO 2017/201325, filed on 18 May 2017, the entire contents of which are hereby incorporated by reference.

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

The present invention also provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes a polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide. In some embodiments, the polynucleotide further comprises a miRNA binding site, e.g., a miRNA binding site that binds.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

microRNAs derive enzymatically from regions of RNA transcripts that fold back on themselves to form short hairpin structures often termed a pre-miRNA (precursor-miRNA). A pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5-prime arm of the pre-miRNA hairpin and “3p” means the microRNA is from the 3-prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′ UTR and/or 3′ UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide long miRNA sequence, to a 19-23 nucleotide long miRNA sequence, or to a 22-nucleotide long miRNA sequence. A miRNA binding site can be complementary to only a portion of a miRNA, e.g., to a portion less than 1, 2, 3, or 4 nucleotides of the full length of a naturally-occurring miRNA sequence, or to a portion less than 1, 2, 3, or 4 nucleotides shorter than a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the polynucleotide. Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

Conversely, miRNA binding sites can be removed from polynucleotide sequences in which they naturally occur to increase protein expression in specific tissues. For example, a binding site for a specific miRNA can be removed from a polynucleotide to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-ld, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′ UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the invention to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′ UTR and/or 3′ UTR of a polynucleotide of the invention.

In some embodiments, the polynucleotide of the invention can include a further negative regulatory element in the 5′ UTR and/or 3′ UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a polynucleotide of the invention to regulate expression of the polynucleotide in the liver. Liver specific miRNA binding sites can be engineered alone or further in combination with immune cell (e.g., APC) miRNA binding sites in a polynucleotide of the invention.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the invention to regulate expression of the polynucleotide in the lung. Lung specific miRNA binding sites can be engineered alone or further in combination with immune cell (e.g., APC) miRNA binding sites in a polynucleotide of the invention.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a polynucleotide of the invention to regulate expression of the polynucleotide in the heart. Heart specific miRNA binding sites can be engineered alone or further in combination with immune cell (e.g., APC) miRNA binding sites in a polynucleotide of the invention.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a polynucleotide of the invention to regulate expression of the polynucleotide in the nervous system. Nervous system specific miRNA binding sites can be engineered alone or further in combination with immune cell (e.g., APC) miRNA binding sites in a polynucleotide of the invention.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a polynucleotide of the invention to regulate expression of the polynucleotide in the pancreas. Pancreas specific miRNA binding sites can be engineered alone or further in combination with immune cell (e.g. APC) miRNA binding sites in a polynucleotide of the invention.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a polynucleotide of the invention to regulate expression of the polynucleotide in the kidney. Kidney specific miRNA binding sites can be engineered alone or further in combination with immune cell (e.g., APC) miRNA binding sites in a polynucleotide of the invention.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polynucleotide of the invention to regulate expression of the polynucleotide in the muscle. Muscle specific miRNA binding sites can be engineered alone or further in combination with immune cell (e.g., APC) miRNA binding sites in a polynucleotide of the invention.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a polynucleotide of the invention to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the invention to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, miRNAs are selected based on expression and abundance in immune cells of the hematopoietic lineage, such as B cells, T cells, macrophages, dendritic cells, and cells that are known to express TLR7/TLR8 and/or able to secrete cytokines such as endothelial cells and platelets. In some embodiments, the miRNA set thus includes miRs that may be responsible in part for the immunogenicity of these cells, and such that a corresponding miR-site incorporation in polynucleotides of the present invention (e.g., mRNAs) could lead to destabilization of the mRNA and/or suppression of translation from these mRNAs in the specific cell type. Non-limiting representative examples include miR-142, miR-144, miR-150, miR-155 and miR-223, which are specific for many of the hematopoietic cells; miR-142, miR150, miR-16 and miR-223, which are expressed in B cells; miR-223, miR-451, miR-26a, miR-16, which are expressed in progenitor hematopoietic cells; and miR-126, which is expressed in plasmacytoid dendritic cells, platelets and endothelial cells. For further discussion of tissue expression of miRs see e.g., Teruel-Montoya, R. et al. (2014) PLoS One 9:e102259; Landgraf, P. et al. (2007) Cell 129:1401-1414; Bissels, U. et al. (2009) RNA 15:2375-2384. Any one miR-site incorporation in the 3′ UTR and/or 5′ UTR may mediate such effects in multiple cell types of interest (e.g., miR-142 is abundant in both B cells and dendritic cells).

In some embodiments, it may be beneficial to target the same cell type with multiple miRs and to incorporate binding sites to each of the 3p and 5p arm if both are abundant (e.g., both miR-142-3p and miR142-5p are abundant in hematopoietic stem cells). Thus, in certain embodiments, polynucleotides of the invention contain two or more (e.g., two, three, four or more) miR bindings sites from: (i) the group consisting of miR-142, miR-144, miR-150, miR-155 and miR-223 (which are expressed in many hematopoietic cells); or (ii) the group consisting of miR-142, miR150, miR-16 and miR-223 (which are expressed in B cells); or the group consisting of miR-223, miR-451, miR-26a, miR-16 (which are expressed in progenitor hematopoietic cells).

In some embodiments, it may also be beneficial to combine various miRs such that multiple cell types of interest are targeted at the same time (e.g., miR-142 and miR-126 to target many cells of the hematopoietic lineage and endothelial cells). Thus, for example, in certain embodiments, polynucleotides of the invention comprise two or more (e.g., two, three, four or more) miRNA bindings sites, wherein: (i) at least one of the miRs targets cells of the hematopoietic lineage (e.g., miR-142, miR-144, miR-150, miR-155 or miR-223) and at least one of the miRs targets plasmacytoid dendritic cells, platelets or endothelial cells (e.g., miR-126); or (ii) at least one of the miRs targets B cells (e.g., miR-142, miR150, miR-16 or miR-223) and at least one of the miRs targets plasmacytoid dendritic cells, platelets or endothelial cells (e.g., miR-126); or (iii) at least one of the miRs targets progenitor hematopoietic cells (e.g., miR-223, miR-451, miR-26a or miR-16) and at least one of the miRs targets plasmacytoid dendritic cells, platelets or endothelial cells (e.g., miR-126); or (iv) at least one of the miRs targets cells of the hematopoietic lineage (e.g., miR-142, miR-144, miR-150, miR-155 or miR-223), at least one of the miRs targets B cells (e.g., miR-142, miR150, miR-16 or miR-223) and at least one of the miRs targets plasmacytoid dendritic cells, platelets or endothelial cells (e.g., miR-126); or any other possible combination of the foregoing four classes of miR binding sites (i.e., those targeting the hematopoietic lineage, those targeting B cells, those targeting progenitor hematopoietic cells and/or those targeting plasmacytoid dendritic cells/platelets/endothelial cells).

In one embodiment, to modulate immune responses, polynucleotides of the present invention can comprise one or more miRNA binding sequences that bind to one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) reduces or inhibits immune cell activation (e.g., B cell activation, as measured by frequency of activated B cells) and/or cytokine production (e.g., production of IL-6, IFN-g and/or TNFa). Furthermore, it has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) can reduce or inhibit an anti-drug antibody (ADA) response against a protein of interest encoded by the mRNA.

In another embodiment, to modulate accelerated blood clearance of a polynucleotide delivered in a lipid-comprising compound or composition, polynucleotides of the invention can comprise one or more miR binding sequences that bind to one or more miRNAs expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miR binding sites reduces or inhibits accelerated blood clearance (ABC) of the lipid-comprising compound or composition for use in delivering the mRNA. Furthermore, it has now been discovered that incorporation of one or more miR binding sites into an mRNA reduces serum levels of anti-PEG anti-IgM (e.g., reduces or inhibits the acute production of IgMs that recognize polyethylene glycol (PEG) by B cells) and/or reduces or inhibits proliferation and/or activation of plasmacytoid dendritic cells following administration of a lipid-comprising compound or composition comprising the mRNA.

In some embodiments, miR sequences may correspond to any known microRNA expressed in immune cells, including but not limited to those taught in US Publication US2005/0261218 and US Publication US2005/0059005, the contents of which are incorporated herein by reference in their entirety. Non-limiting examples of miRs expressed in immune cells include those expressed in spleen cells, myeloid cells, dendritic cells, plasmacytoid dendritic cells, B cells, T cells and/or macrophages. For example, miR-142-3p, miR-142-5p, miR-16, miR-21, miR-223, miR-24 and miR-27 are expressed in myeloid cells, miR-155 is expressed in dendritic cells, B cells and T cells, miR-146 is upregulated in macrophages upon TLR stimulation and miR-126 is expressed in plasmacytoid dendritic cells. In certain embodiments, the miR(s) is expressed abundantly or preferentially in immune cells. For example, miR-142 (miR-142-3p and/or miR-142-5p), miR-126 (miR-126-3p and/or miR-126-5p), miR-146 (miR-146-3p and/or miR-146-5p) and miR-155 (miR-155-3p and/or miR155-5p) are expressed abundantly in immune cells. These microRNA sequences are known in the art and, thus, one of ordinary skill in the art can readily design binding sequences or target sequences to which these microRNAs will bind based upon Watson-Crick complementarity.

Accordingly, in various embodiments, polynucleotides of the present invention comprise at least one microRNA binding site for a miR selected from the group consisting of miR-142, miR-146, miR-155, miR-126, miR-16, miR-21, miR-223, miR-24 and miR-27. In another embodiment, the mRNA comprises at least two miR binding sites for microRNAs expressed in immune cells. In various embodiments, the polynucleotide of the invention comprises 1-4, one, two, three or four miR binding sites for microRNAs expressed in immune cells. In another embodiment, the polynucleotide of the invention comprises three miR binding sites. These miR binding sites can be for microRNAs selected from the group consisting of miR-142, miR-146, miR-155, miR-126, miR-16, miR-21, miR-223, miR-24, miR-27, and combinations thereof. In one embodiment, the polynucleotide of the invention comprises two or more (e.g., two, three, four) copies of the same miR binding site expressed in immune cells, e.g., two or more copies of a miR binding site selected from the group of miRs consisting of miR-142, miR-146, miR-155, miR-126, miR-16, miR-21, miR-223, miR-24, miR-27.

In one embodiment, the polynucleotide of the invention comprises three copies of the same miRNA binding site. In certain embodiments, use of three copies of the same miR binding site can exhibit beneficial properties as compared to use of a single miRNA binding site. Non-limiting examples of sequences for 3′ UTRs containing three miRNA bindings sites are shown in SEQ ID NO: 155 (three miR-142-3p binding sites) and SEQ ID NO: 157 (three miR-142-5p binding sites).

In another embodiment, the polynucleotide of the invention comprises two or more (e.g., two, three, four) copies of at least two different miR binding sites expressed in immune cells. Non-limiting examples of sequences of 3′ UTRs containing two or more different miR binding sites are shown in SEQ ID NO:111 (one miR-142-3p binding site and one miR-126-3p binding site), SEQ ID NO: 158 (two miR-142-5p binding sites and one miR-142-3p binding sites), and SEQ ID NO: 161 (two miR-155-5p binding sites and one miR-142-3p binding sites).

In another embodiment, the polynucleotide of the invention comprises at least two miR binding sites for microRNAs expressed in immune cells, wherein one of the miR binding sites is for miR-142-3p. In various embodiments, the polynucleotide of the invention comprises binding sites for miR-142-3p and miR-155 (miR-155-3p or miR-155-5p), miR-142-3p and miR-146 (miR-146-3 or miR-146-5p), or miR-142-3p and miR-126 (miR-126-3p or miR-126-5p).

In another embodiment, the polynucleotide of the invention comprises at least two miR binding sites for microRNAs expressed in immune cells, wherein one of the miR binding sites is for miR-126-3p. In various embodiments, the polynucleotide of the invention comprises binding sites for miR-126-3p and miR-155 (miR-155-3p or miR-155-5p), miR-126-3p and miR-146 (miR-146-3p or miR-146-5p), or miR-126-3p and miR-142 (miR-142-3p or miR-142-5p).

In another embodiment, the polynucleotide of the invention comprises at least two miR binding sites for microRNAs expressed in immune cells, wherein one of the miR binding sites is for miR-142-5p. In various embodiments, the polynucleotide of the invention comprises binding sites for miR-142-5p and miR-155 (miR-155-3p or miR-155-5p), miR-142-5p and miR-146 (miR-146-3 or miR-146-5p), or miR-142-5p and miR-126 (miR-126-3p or miR-126-5p).

In yet another embodiment, the polynucleotide of the invention comprises at least two miR binding sites for microRNAs expressed in immune cells, wherein one of the miR binding sites is for miR-155-5p. In various embodiments, the polynucleotide of the invention comprises binding sites for miR-155-5p and miR-142 (miR-142-3p or miR-142-5p), miR-155-5p and miR-146 (miR-146-3 or miR-146-5p), or miR-155-5p and miR-126 (miR-126-3p or miR-126-5p).

miRNA can also regulate complex biological processes such as angiogenesis (e.g., miR-132) (Anand and Cheresh Curr Opin Hematol 2011 18:171-176). In the polynucleotides of the invention, miRNA binding sites that are involved in such processes can be removed or introduced, to tailor the expression of the polynucleotides to biologically relevant cell types or relevant biological processes. In this context, the polynucleotides of the invention are defined as auxotrophic polynucleotides.

In some embodiments, a polynucleotide of the invention comprises a miRNA binding site, wherein the miRNA binding site comprises one or more nucleotide sequences selected from Table 3C or Table 4B, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 3C or Table 4B, including any combination thereof.

In some embodiments, the miRNA binding site binds to miR-142 or is complementary to miR-142. In some embodiments, the miR-142 comprises SEQ ID NO:114. In some embodiments, the miRNA binding site binds to miR-142-3p or miR-142-5p. In some embodiments, the miR-142-3p binding site comprises SEQ ID NO:116. In some embodiments, the miR-142-5p binding site comprises SEQ ID NO:118. In some embodiments, the miRNA binding site comprises a nucleotide sequence at least 80%, at least 85%, at least 90%, at least 95%, or 100% identical to SEQ ID NO:116 or SEQ ID NO:118.

In some embodiments, the miRNA binding site binds to miR-126 or is complementary to miR-126. In some embodiments, the miR-126 comprises SEQ ID NO: 119. In some embodiments, the miRNA binding site binds to miR-126-3p or miR-126-5p. In some embodiments, the miR-126-3p binding site comprises SEQ ID NO: 121. In some embodiments, the miR-126-5p binding site comprises SEQ ID NO: 123. In some embodiments, the miRNA binding site comprises a nucleotide sequence at least 80%, at least 85%, at least 90%, at least 95%, or 100% identical to SEQ ID NO: 121 or SEQ ID NO: 123.

In one embodiment, the 3′ UTR comprises two miRNA binding sites, wherein a first miRNA binding site binds to miR-142 and a second miRNA binding site binds to miR-126. In a specific embodiment, the 3′ UTR binding to miR-142 and miR-126 comprises, consists, or consists essentially of the sequence of SEQ ID NO: 163.

TABLE 3C
miR-142, miR-126, and miR-142 and miR-126 binding sites

| SEQ ID NO. | Description | Sequence |
|---|---|---|
| 114 | miR-142 | GACAGUGCAGUCACCCAUAAAGUAGA |
| | | AAGCACUACUAACAGCACUGGAGGGU |
| | | GUAGUGUUUCCUACUUUAUGGAUGAG |
| | | UGUACUGUG |
| 115 | miR-142-3p | uguaguguuuccuacuuuaugga |
| 116 | miR-142-3p binding | uccauaaaguaggaaacacuaca |
| | site | |
| 117 | miR-142-5p | cauaaaguagaaagcacuacu |
| 118 | miR-142-5p binding | aguagugcuuucuacuuuaug |
| | site | |
| 119 | miR-126 | CGCUGGCGACGGGACAUUAUUACUUU |
| | | UGGUACGCGCUGUGACACUUCAAACU |
| | | CGUACCGUGAGUAAUAAUGCGCCGUC |
| | | CACGGCA |
| 120 | miR-126-3p | UCGUACCGUGAGUAAUAAUGCG |
| 121 | miR-126-3p binding | CGCAUUAUUACUCACGGUACGA |
| | site | |
| 122 | miR-126-5p | CAUUAUUACUUUUGGUACGCG |
| 123 | miR-126-5p binding | CGCGUACCAAAAGUAAUAAUG |
| | site | |

In some embodiments, a miRNA binding site is inserted in the polynucleotide of the invention in any position of the polynucleotide (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide. In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention. In some embodiments, a miRNA binding site is inserted within the 3′ UTR immediately following the stop codon of the coding region within the polynucleotide of the invention, e.g., mRNA. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s). In some embodiments, three non-limiting examples of possible insertion sites for a miR in a 3′ UTR are shown in SEQ ID NOs: 162, 163, and 164, which show a 3′ UTR sequence with a miR-142-3p site inserted in one of three different possible insertion sites, respectively, within the 3′ UTR.

In some embodiments, one or more miRNA binding sites can be positioned within the 5′ UTR at one or more possible insertion sites. For example, three non-limiting examples of possible insertion sites for a miR in a 5′ UTR are shown in SEQ ID NOs: 165, 166, or 167, which show a 5′ UTR sequence with a miR-142-3p site inserted into one of three different possible insertion sites, respectively, within the 5′ UTR.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG (SEQ ID NO:124), UGAUAGUAA (SEQ ID NO:125), UAAUGAUAG (SEQ ID NO:126), UGAUAAUAA (SEQ ID NO:127), UGAUAGUAG (SEQ ID NO:128), UAAUGAUGA (SEQ ID NO:129), UAAUAGUAG (SEQ ID NO:130), UGAUGAUGA (SEQ ID NO:131), UAAUAAUAA (SEQ ID NO:132), and UAGUAGUAG (SEQ ID NO:133). Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon. Non-limiting examples of sequences of 3′ UTR having three stop codons and a single miR-142-3p binding site located at different positions downstream of the final stop codon are shown in SEQ ID NOs: 151, 162, 163, and 164.

TABLE 4B
5′ UTRs, 3′UTRs, miR sequences, and miR binding sites

| SEQ ID NO: | Sequence |
|---|---|
| 134 | GCUGGAGCCUCGGUGGCCAUGCUUCUUGCCCCUUGGGCCUCCCCCCAGCCCCUCCUC |
| | CCCUUCCUGCACCCGUACCCCCUCCAUAAAGUAGGAAACACUACAGUGGUCUUUGAA |
| | UAAAGUCUGAGUGGGCGGC |
| | (3′ UTR with miR 142-3p binding site) |
| 116 | UCCAUAAAGUAGGAAACACUACA |
| | (miR 142-3p binding site) |
| 115 | UGUAGUGUUUCCUACUUUAUGGA |
| | (miR 142-3p sequence) |
| 117 | CAUAAAGUAGAAAGCACUACU |
| | (miR 142-5p sequence) |
| 135 | CCUCUGAAAUUCAGUUCUUCAG |
| | (miR 146-3p sequence) |
| 136 | UGAGAACUGAAUUCCAUGGGUU |
| | (miR 146-5p sequence) |
| 137 | CUCCUACAUAUUAGCAUUAACA |
| | (miR 155-3p sequence) |
| 138 | UUAAUGCUAAUCGUGAUAGGGGU |
| | (miR 155-5p sequence) |
| 120 | UCGUACCGUGAGUAAUAAUGCG |
| | (miR 126-3p sequence) |
| 122 | CAUUAUUACUUUUGGUACGCG |
| | (miR 126-5p sequence) |
| 139 | CCAGUAUUAACUGUGCUGCUGA |
| | (miR 16-3p sequence) |
| 140 | UAGCAGCACGUAAAUAUUGGCG |
| | (miR 16-5p sequence) |
| 141 | CAACACCAGUCGAUGGGCUGU |
| | (miR 21-3p sequence) |
| 142 | UAGCUUAUCAGACUGAUGUUGA |
| | (miR 21-5p sequence) |
| 143 | UGUCAGUUUGUCAAAUACCCCA |
| | (miR 223-3p sequence) |
| 144 | CGUGUAUUUGACAAGCUGAGUU |
| | (miR 223-5p sequence) |
| 145 | UGGCUCAGUUCAGCAGGAACAG |
| | (miR 24-3p sequence) |
| 146 | UGCCUACUGAGCUGAUAUCAGU |
| | (miR 24-5p sequence) |
| 147 | UUCACAGUGGCUAAGUUCCGC |
| | (miR 27-3p sequence) |
| 148 | AGGGCUUAGCUGCUUGUGAGCA |
| | (miR 27-5p sequence) |
| 121 | CGCAUUAUUACUCACGGUACGA |
| | (miR 126-3p binding site) |
| 149 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCC [symbol] GUGG |
| | UCUUUGAAUAAAGUCUGAGUGGGCGGC |
| | (3′ UTR with miR 126-3p binding site) |
| 150 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCGUGGUCUUUGAAUAAAGUCUGAGUGG |
| | GCGGC |
| | (3′ UTR, no miR binding sites) |
| 151 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCUCCAUAAAGUAGGAAACACUACAGUG |
| | GUCUUUGAAUAAAGUCUGAGUGGGCGGC |
| | (3′ UTR with miR 142-3p binding site) |
| 111 | UGAUAAUAGUCCAUAAAGUAGGAAACACUACAGCUGGAGCCUCGGUGGCCAUGCUUC |
| | UUGCCCCUUGGGCCUCCCCCCAGCCCCUCCUCCCCUUCCUGCACCCGUACCCCC [symbol] |
| | [symbol] GUGGUCUUUGAAUAAAGUCUGAGUGGGGGC |
| | (3′ UTR with miR 142-3p and miR 126-3p binding sites variant 1) |
| 153 | UUAAUGCUAAUUGUGAUAGGGGU |
| | (miR 155-5p sequence) |
| 154 | ACCCCUAUCACAAUUAGCAUUAA |
| | (miR 155-5p binding site) |
| 155 | UGAUAAUAGUCCAUAAAGUAGGAAACACUACAGCUGGAGCCUCGGUGGCCAUGCUUC |
| | UUGCCCCUUGGGCCUCCAUAAAGUAGGAAACACUACAUCCCCCCAGCCCCUCCUCCC |
| | CUUCCUGCACCCGUACCCCCUCCAUAAAGUAGGAAACACUACAGUGGUCUUUGAAUA |
| | AAGUCUGAGUGGGCGGC |
| | (3′ UTR with 3 miR 142-3p binding sites) |
| 156 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCC [symbol] GUGGU |
| | CUUUGAAUAAAGUCUGAGUGGGCGGC |
| | (3′ UTR with miR 142-5p binding site) |
| 157 | UGAUAAUAG [symbol] GCUGGAGCCUCGGUGGCCAUGCUUCUU |
| | GCCCCUUGGGCC [symbol] UCCCCCCAGCCCCUCCUCCCCUUC |
| | CUGCACCCGUACCCCC [symbol] GUGGUCUUUGAAUAAAGUCU |
| | GAGUGGGCGGC |
| | (3′ UTR with 3 miR 142-5p binding sites) |
| 158 | UGAUAAUAG [symbol] GCUGGAGCCUCGGUGGCCAUGCUUCUU |
| | GCCCCUUGGGCCUCCAUAAAGUAGGAAACACUACAUCCCCCCAGCCCCUCCUCCCCU |
| | UCCUGCACCCGUACCCCC [symbol] GUGGUCUUUGAAUAAAGU |
| | CUGAGUGGGCGGC |
| | (3′ UTR with 2 miR 142-5p binding sites and 1 miR 142-3p binding site) |
| 159 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCACCCCUAUCACAAUUAGCAUUAAGUG |
| | GUCUUUGAAUAAAGUCUGAGUGGGCGGC |
| | (3′ UTR with miR 155-5p binding site) |
| 160 | UGAUAAUAGACCCCUAUCACAAUUAGCAUUAAGCUGGAGCCUCGGUGGCCAUGCUUC |
| | UUGCCCCUUGGGCCACCCCUAUCACAAUUAGCAUUAAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 3 miR 155-5p binding sites) |
| 161 | UGAUAAUAGACCCCUAUCACAAUUAGCAUUAAGCUGGAGCCUCGGUGGCCAUGCUUC |
| | UUGCCCCUUGGGCCUCCAUAAAGUAGGAAACACUACAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 2 miR 155-5p binding sites and 1 miR 142-3p binding site) |
| 162 | UGAUAAUAGUCCAUAAAGUAGGAAACACUACAGCUGGAGCCUCGGUGGCCAUGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P1 insertion) |
| 163 | UGAUAAUAGGCUGGAGCCUCGGUGGCUCCAUAAAGUAGGAAACACUACACAUGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P2 insertion) |
| 164 | UGAUAAUAGGCUGGAGCCUCGGUGGCCAUGCUUCUUGCCCCUUGGGCCUCCAUAAAG |
| | UAGGAAACACUACAUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P3 insertion) |
| 118 | AGUAGUGCUUUCUACUUUAUG |
| | (miR-142-5p binding site) |
| 114 | GACAGUGCAGUCACCCAUAAAGUAGAAAGCACUACUAACAGCACUGGAGGGUGUAGU |
| | GUUUCCUACUUUAUGGAUGAGUGUACUGUG |
| | (miR-142) |
| 185 | GGGAAAUAAGAGAGAAAAGAAGAGUAAGAAGAAAUAUAAGAGCCACC |
| | (5′ UTR) |
| 165 | GGGAAAUAAGAGUCCAUAAAGUAGGAAACACUACAAGAAAAGAAGAGUAAGAAGAAA |
| | UAUAAGAGCCACC |
| | (5′ UTR with miR142-3p binding site at position p1) |
| 166 | GGGAAAUAAGAGAGAAAAGAAGAGUAAUCCAUAAAGUAGGAAACACUACAGAAGAAA |
| | UAUAAGAGCCACC |
| | (5′ UTR with miR142-3p binding site at position p2) |
| 167 | GGGAAAUAAGAGAGAAAAGAAGAGUAAGAAGAAAUAUAAUCCAUAAAGUAGGAAACA |
| | CUACAGAGCCACC |
| | (5′ UTR with miR142-3p binding site at position p3) |
| 168 | ACCCCUAUCACAAUUAGCAUUAA |
| | (miR 155-5p binding site) |
| 169 | UGAUAAUAG [symbol] GCUGGAGCCUCGGUGGCCAUGCUUCUU |
| | GCCCCUUGGGCC [symbol] UCCCCCCAGCCCCUCUCCCCUUCC |
| | UGCACCCGUACCCCC [symbol] GUGGUCUUUGAAUAAAGUCUG |
| | AGUGGGCGGC |
| | (3′ UTR with 3 miR 142-5p binding sites) |
| 170 | UGAUAAUAGGCUGGAGCCUCGGUGGCCAUGCUUCUUGCCCCUUCCAUAAAGUAGGAA |
| | ACACUACAUGGGCCUCCCCCCAGCCCCUCCUCCCCUUCCUGCACCCGUACCCCCGUG |
| | GUCUUUGAAUAAAGUCUGAGUGGGCGGC |
| | (3′UTR including miR142-3p binding site) |
| 171 | UGAUAAUAGGCUGGAGCCUCGGUGGCCAUGCUUCUUGCCCCUUGGGCCUCCCCCCAG |
| | UCCAUAAAGUAGGAAACACUACACCCCUCCUCCCCUUCCUGCACCCGUACCCCCGUG |
| | GUCUUUGAAUAAAGUCUGAGUGGGGGC |
| | (3′UTR including miR142-3p binding site) |
| 172 | UGAUAAUAGGCUGGAGCCUCGGUGGCCAUGCUUCUUGCCCCUUGGGCCUCCCCCCAG |
| | CCCCUCCUCCCCUUCUCCAUAAAGUAGGAAACACUACACUGCACCCGUACCCCCGUG |
| | GUCUUUGAAUAAAGUCUGAGUGGGCGGC |
| | (3′UTR including miR142-3p binding site) |
| 173 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCGUGGUCUUUGAAUAAAGUUCCAUAAA |
| | GUAGGAAACACUACACUGAGUGGGCGGC |
| | (3′UTR including miR142-3p binding site) |
| 174 | UGAUAAUAGUCCAUAAAGUAGGAAACACUACAGCUGGAGCCUCGGUGGCCUAGCUUC |
| | UUGCCCCUUGGGCCUCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-3p and miR 126-3p binding sites variant 2) |
| 175 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCGUGGUCUUUGAAUAAAGUCUGAGUGG |
| | GCGGC |
| | (3′ UTR, no miR binding sites variant 2) |
| 186 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCUCCAUAAAGUAGGAAACACUACAGUG |
| | GUCUUUGAAUAAAGUCUGAGUGGGCGGC |
| | (3′ UTR with miR 142-3p binding site variant 3) |
| 177 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCC [symbol] GUGG |
| | UCUUUGAAUAAAGUCUGAGUGGGCGGC |
| | (3′ UTR with miR 126-3p binding site variant 3) |
| 178 | UGAUAAUAGUCCAUAAAGUAGGAAACACUACAGCUGGAGCCUCGGUGGCCUAGCUUC |
| | UUGCCCCUUGGGCCUCCAUAAAGUAGGAAACACUACAUCCCCCCAGCCCCUCCUCCC |
| | CUUCCUGCACCCGUACCCCCUCCAUAAAGUAGGAAACACUACAGUGGUCUUUGAAUA |
| | AAGUCUGAGUGGGCGGC |
| | (3′ UTR with 3 miR 142-3p binding sites variant 2) |
| 179 | UGAUAAUAGUCCAUAAAGUAGGAAACACUACAGCUGGAGCCUCGGUGGCCUAGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′UTR with miR 142-3p binding site, P1 insertion variant 2) |
| 180 | UGAUAAUAGGCUGGAGCCUCGGUGGCUCCAUAAAGUAGGAAACACUACACUAGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′UTR with miR 142-3p binding site, P2 insertion variant 2) |
| 181 | UGAUAAUAGGCUGGAGCCUCGGUGGCCUAGCUUCUUGCCCCUUGGGCCUCCAUAAAG |
| | UAGGAAACACUACAUCCCCCCAGCCCCUCCUCCCCUUCCUGCACCCGUACCCCCGUG |
| | GUCUUUGAAUAAAGUCUGAGUGGGCGGC |
| | (3′UTR with miR 142-3p binding site, P3 insertion variant 2) |
| 182 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCACCCCUAUCACAAUUAGCAUUAAGUG |
| | GUCUUUGAAUAAAGUCUGAGUGGGCGGC |
| | (3′UTR with miR 155-5p binding site variant 2) |
| 183 | UGAUAAUAGACCCCUAUCACAAUUAGCAUUAAGCUGGAGCCUCGGUGGCCUAGCUUC |
| | UUGCCCCUUGGGCCACCCCUAUCACAAUUAGCAUUAAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 3 miR 155-5p binding sites variant 2) |
| 184 | UGAUAAUAGACCCCUAUCACAAUUAGCAUUAAGCUGGAGCCUCGGUGGCCUAGCUUC |
| | UUGCCCCUUGGGCCUCCAUAAAGUAGGAAACACUACAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′UTR with 2 miR 155-5p binding sites and 1 miR 142-3p binding site variant 2) |

Stop codon = bold
miR 142-3p binding site = underline
miR 126-3p binding site = bold underline
miR 155-5p binding site = italicized
miR 142-5p binding site = italicized and bold underline

In one embodiment, the polynucleotide of the invention comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 116. In one embodiment, the 3′ UTR of the mRNA comprising the miR-142-3p microRNA binding site comprises the sequence shown in SEQ ID NO: 134.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 121. In one embodiment, the 3′ UTR of the mRNA of the invention comprising the miR-126-3p microRNA binding site comprises the sequence shown in SEQ ID NO: 149.

Non-limiting exemplary sequences for miRs to which a microRNA binding site(s) of the disclosure can bind include the following: miR-142-3p (SEQ ID NO: 115), miR-142-5p (SEQ ID NO: 117), miR-146-3p (SEQ ID NO: 135), miR-146-5p (SEQ ID NO: 136), miR-155-3p (SEQ ID NO: 137), miR-155-5p (SEQ ID NO: 138), miR-126-3p (SEQ ID NO: 120), miR-126-5p (SEQ ID NO: 122), miR-16-3p (SEQ ID NO: 139), miR-16-5p (SEQ ID NO: 140), miR-21-3p (SEQ ID NO: 141), miR-21-5p (SEQ ID NO: 142), miR-223-3p (SEQ ID NO: 143), miR-223-5p (SEQ ID NO: 144), miR-24-3p (SEQ ID NO: 145), miR-24-5p (SEQ ID NO: 146), miR-27-3p (SEQ ID NO: 147) and miR-27-5p (SEQ ID NO: 148). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a polynucleotide of the present invention (e.g., and mRNA, e.g., the 3′ UTR thereof) can comprise at least one miRNA binding site to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA binding site for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′ UTR to enhance microRNA mediated gene regulation.

At least one miRNA binding site can be engineered into the 3′ UTR of a polynucleotide of the invention. In this context, at least two, at least three, at least four, at least five, at least six, at least seven, at least eight, at least nine, at least ten, or more miRNA binding sites can be engineered into a 3′ UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., myeloid cells, endothelial cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′ UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′ UTR. In another non-limiting example, a miRNA binding site can be engineered near the 5′ terminus of the 3′ UTR and near the 3′ terminus of the 3′ UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a ionizable lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a polynucleotide of the invention described herein.

In another embodiment, a miRNA sequence in the 5′ UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a polynucleotide of the invention can include at least one miRNA to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA to dampen expression of the encoded polypeptide in a tissue or cell of interest. As a non-limiting example, a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include miR-142-5p, miR-142-3p, miR-146a-5p, and miR-146-3p.

In one embodiment, a polynucleotide of the invention comprises at least one miRNA sequence in a region of the polynucleotide that can interact with a RNA binding protein. In some embodiments, the polynucleotide of the invention (e.g., a RNA, e.g., an mRNA) comprising (i) a sequence-optimized nucleotide sequence (e.g., an ORF) encoding an immune checkpoint inhibitor polypeptide (e.g., the wild-type sequence, functional fragment, or variant thereof) and (ii) a miRNA binding site (e.g., a miRNA binding site that binds to miR-142) and/or a miRNA binding site that binds to miR-126.

### IVT Polynucleotide Architecture

In some embodiments, the polynucleotide of the present disclosure comprising an mRNA encoding an IL-2 polypeptide, or a GM-CSF polypeptide is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded IL-2 polypeptide, or a GM-CSF polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding an IL-2 polypeptide, or a GM-CSF polypeptide can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of IL-2, or GM-CSF polypeptide or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

In some embodiments, the mRNA encoding an IL-2 polypeptide, or a GM-CSF polypeptide comprises a poly A tail, e.g., a poly A tail sequence provided in Table 5A. In some embodiments, the poly A tail is 50-150 (SEQ ID NO: 189), 75-150 (SEQ ID NO: 190), 85-150 (SEQ ID NO: 191), 90-150 (SEQ ID NO: 192), 90-120 (SEQ ID NO: 193), 90-130 (SEQ ID NO: 194), or 90-150 (SEQ ID NO: 192) nucleotides in length. In some embodiments, the poly A tail is 100 nucleotides in length (SEQ ID NO:29).

SEQ ID NO: 29:
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding the IL-2 polypeptide, or the ORF encoding the GM-CSF polypeptide or both. In some embodiments, the UTR is heterologous to the ORF encoding the GM-CSF polypeptide. In some embodiments, the UTR is heterologous to the ORF encoding the IL-2 polypeptide. In some embodiments, the UTR is heterologous to the ORF encoding the GM-CSF polypeptide and the ORF encoding the IL-2 polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively. Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:87), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-⊕) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1). In some embodiments, the 5′ UTR is selected from the group consisting of a β-globin 5′ UTR; a 5′UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs. In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′ UTR and/or a 3′ UTR selected from any of the UTRs disclosed herein. In some embodiments, the 5′ UTR comprises:

5′ UTR-001 (Upstream UTR)
(SEQ ID NO: 185)
(GGGAAAUAAGAGAGAAAAGAAGAGUAAGAAGAAAUAUAAGAGCCACC);
5′ UTR-002 (Upstream UTR)
(SEQ ID NO: 89)
(GGGAGAUCAGAGAGAAAAGAAGAGUAAGAAGAAAUAUAAGAGCCACC);
5′ UTR-003 (Upstream UTR);
(See WO2016/100812)
5′ UTR-004 (Upstream UTR)
(SEQ ID NO: 90)
(GGGAGACAAGCUUGGCAUUCCGGUACUGUUGGUAAAGCCACC);
5′ UTR-005 (Upstream UTR)
(SEQ ID NO: 91)
(GGGAGAUCAGAGAGAAAAGAAGAGUAAGAAGAAAUAUAAGAGCCACC);
5′ UTR-006 (Upstream UTR);
(See WO2016/100812)
5′ UTR-007 (Upstream UTR)
(SEQ ID NO: 92)
(GGGAGACAAGCUUGGCAUUCCGGUACUGUUGGUAAAGCCACC);
5′ UTR-008 (Upstream UTR)
(SEQ ID NO: 93)
(GGGAAUUAACAGAGAAAAGAAGAGUAAGAAGAAAUAUAAGAGCCACC);
5′ UTR-009 (Upstream UTR)
(SEQ ID NO: 94)
(GGGAAAUUAGACAGAAAAGAAGAGUAAGAAGAAAUAUAAGAGCCACC);
5′ UTR-010, Upstream
(SEQ ID NO: 95)
(GGGAAAUAAGAGAGUAAAGAACAGUAAGAAGAAAUAUAAGAGCCACC);
5′ UTR-011 (Upstream UTR)
(SEQ ID NO: 96)
(GGGAAAAAAGAGAGAAAAGAAGACUAAGAAGAAAUAUAAGAGCCACC);
5′ UTR-012 (Upstream UTR)
(SEQ ID NO: 97)
(GGGAAAUAAGAGAGAAAAGAAGAGUAAGAAGAUAUAUAAGAGCCACC);
5′ UTR-013 (Upstream UTR)
(SEQ ID NO: 98)
(GGGAAAUAAGAGACAAAACAAGAGUAAGAAGAAAUAUAAGAGCCACC);
5′ UTR-014 (Upstream UTR)
(SEQ ID NO: 99)
(GGGAAAUUAGAGAGUAAAGAACAGUAAGUAGAAUUAAAAGAGCCACC);
5′ UTR-015 (Upstream UTR)
(SEQ ID NO: 100)
(GGGAAAUAAGAGAGAAUAGAAGAGUAAGAAGAAAUAUAAGAGCCACC);
5′ UTR-016 (Upstream UTR)
(SEQ ID NO: 101)
(GGGAAAUAAGAGAGAAAAGAAGAGUAAGAAGAAAAUUAAGAGCCACC);
5′ UTR-017 (Upstream UTR);
(SEQ ID NO: 102)
(GGGAAAUAAGAGAGAAAAGAAGAGUAAGAAGAAAUUUAAGAGCCACC);
or
5′ UTR-018 (Upstream UTR) 5′ UTR
(SEQ ID NO: 88)
(UCAAGCUUUUGGACCCUCGUACAGAAGCUAAUACGACUCACUAUAGGGAAAUAA
GAGAGAAAAGAAGAGUAAGAAGAAAUAUAAGAGCCACC).
In some embodiments, the 3′ UTR comprises:
142-3p 3′ UTR (UTR including miR 142-3p binding site)
(SEQ ID NO: 104)
(UGAUAAUAGUCCAUAAAGUAGGAAACACUACAGCUGGAGCCUCGGUGGCCAUGC
UUCUUGCCCCUUGGGCCUCCCCCCAGCCCCUCCUCCCCUUCCUGCACCCGUACCCC
CGUGGUCUUUGAAUAAAGUCUGAGUGGGCGGC);
142-3p 3′ UTR (UTR including miR 142-3p binding site)
(SEQ ID NO: 105)
(UGAUAAUAGGCUGGAGCCUCGGUGGCUCCAUAAAGUAGGAAACACUACACAUGC
UUCUUGCCCCUUGGGCCUCCCCCCAGCCCCUCCUCCCCUUCCUGCACCCGUACCCC
CGUGGUCUUUGAAUAAAGUCUGAGUGGGCGGC);
or
142-3p 3′ UTR (UTR including miR 142-3p binding site)
(SEQ ID NO: 106)
(UGAUAAUAGGCUGGAGCCUCGGUGGCCAUGCUUCUUGCCCCUUCCAUAAAGUAG
GAAACACUACAUGGGCCUCCCCCCAGCCCCUCCUCCCCUUCCUGCACCCGUACCCC
CGUGGUCUUUGAAUAAAGUCUGAGUGGGCGGC);
142-3p 3′ UTR (UTR including miR 142-3p binding site)
(SEQ ID NO: 107)
(UGAUAAUAGGCUGGAGCCUCGGUGGCCAUGCUUCUUGCCCCUUGGGCCUCCCCCC
AGUCCAUAAAGUAGGAAACACUACACCCCUCCUCCCCUUCCUGCACCCGUACCCC
CGUGGUCUUUGAAUAAAGUCUGAGUGGGCGGC);
142-3p 3′ UTR (UTR including miR 142-3p binding site)
(SEQ ID NO: 108)
(UGAUAAUAGGCUGGAGCCUCGGUGGCCAUGCUUCUUGCCCCUUGGGCCUCCCCCC
AGCCCCUCCUCCCCUUCUCCAUAAAGUAGGAAACACUACACUGCACCCGUACCCC
CGUGGUCUUUGAAUAAAGUCUGAGUGGGCGGC);
142-3p 3′ UTR (UTR including miR 142-3p binding site)
(SEQ ID NO: 109)
(UGAUAAUAGGCUGGAGCCUCGGUGGCCAUGCUUCUUGCCCCUUGGGCCUCCCCCC
AGCCCCUCCUCCCCUUCCUGCACCCGUACCCCCUCCAUAAAGUAGGAAACACUAC
AGUGGUCUUUGAAUAAAGUCUGAGUGGGCGGC).
142-3p 3′ UTR (UTR including miR 142-3p binding site)
(SEQ ID NO: 110)
(UGAUAAUAGGCUGGAGCCUCGGUGGCCAUGCUUCUUGCCCCUUGGGCCUCCCCCC
AGCCCCUCCUCCCCUUCCUGCACCCGUACCCCCGUGGUCUUUGAAUAAAGUUCCA
UAAAGUAGGAAACACUACACUGAGUGGGGGC);
3′ UTR-018;
(See SEQ ID NO: 150)
3′ UTR (miR142 and miR126 binding sites variant 1)
(SEQ ID NO: 111)
(UGAUAAUAGUCCAUAAAGUAGGAAACACUACAGCUGGAGCCUCGGUGGCCAUGC
UUCUUGCCCCUUGGGCCUCCCCCCAGCCCCUCCUCCCCUUCCUGCACCCGUACCCC
CCGCAUUAUUACUCACGGUACGAGUGGUCUUUGAAUAAAGUCUGAGUGGGCGGC)
3′ UTR (miR142 and miR126 binding sites variant 2)
(SEQ ID NO: 112)
(UGAUAAUAGUCCAUAAAGUAGGAAACACUACAGCUGGAGCCUCGGUGGCCUAGC
UUCUUGCCCCUUGGGCCUCCCCCCAGCCCCUCCUCCCCUUCCUGCACCCGUACCCC
CCGCAUUAUUACUCACGGUACGAGUGGUCUUUGAAUAAAGUCUGAGUGGGCGGC);
or
3′UTR (miR142-3p binding site variant 3)
(SEQ ID NO: 186)
UGAUAAUAGGCUGGAGCCUCGGUGGCCUAGCUUCUUGCCCCUUGGGCCUCCCCCC
AGCCCCUCCUCCCCUUCCUGCACCCGUACCCCCUCCAUAAAGUAGGAAACACUAC
AGUGGUCUUUGAAUAAAGUCUGAGUGGGCGGC.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a 5′ UTR and/or 3′ UTR sequence provided herein. In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs: 185, 88-102, or 165-167 and/or 3′ UTR sequences comprises any of SEQ ID NOs:104-112, 150, 151, or 178, and any combination thereof.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NO: 185, SEQ ID NO:193, SEQ ID NO:39, or SEQ ID NO:194 and/or 3′ UTR sequences comprises any of SEQ ID NO:150, SEQ ID NO:175, SEQ ID NO:195, SEQ ID NO:196, SEQ ID NO: 186, SEQ ID NO:177, SEQ ID NO:111, or SEQ ID NO:178, and any combination thereof.

In some embodiments, the 5′ UTR comprises an amino acid sequence set forth in Table 4B. In some embodiments, the 3′ UTR comprises an amino acid sequence set forth in Table 4B. In some embodiments, the 5′ UTR comprises an amino acid sequence set forth in Table 4B and the 3′ UTR comprises an amino acid sequence set forth in Table 4B.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

### Regions Having a 5′ Cap

The disclosure also includes a polynucleotide that comprises both a 5′ Cap and a polynucleotide of the present invention (e.g., a polynucleotide comprising a nucleotide sequence encoding an IL-2 polypeptide, or a GM-CSF polypeptide).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding an immune checkpoint inhibitor polypeptide) incorporate a cap moiety.

In some embodiments, polynucleotides of the present invention (e.g., a polynucleotide comprising a nucleotide sequence encoding an immune checkpoint inhibitor polypeptide) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-0 atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding an IL-2 polypeptide, or a GM-CSF polypeptide) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present invention are those that, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′ cap analog structures known in the art. Cap structures include, but are not limited to, 7mG(5′)ppp(5′)N, pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′ fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding an IL-2 polypeptide, or a GM-CSF polypeptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:29).

(SEQ ID NO: 29)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO: 187).

(SEQ ID NO: 187)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The invention also includes a polynucleotide that comprises both a start codon region and the polynucleotide described herein (e.g., a polynucleotide comprising a nucleotide sequence encoding an IL-2 polypeptide, or a GM-CSF polypeptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding an IL-2 polypeptide, or a GM-CSF polypeptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

### Methods of Making Polynucleotides

The present disclosure also provides methods for making a polynucleotide disclosed herein or a complement thereof. In some aspects, a polynucleotide (e.g., an mRNA) disclosed herein encoding an IL-2 molecule and/or a GM-CSF molecule can be constructed using in vitro transcription.

In other aspects, a polynucleotide (e.g., an mRNA) disclosed herein encoding an IL-2 molecule and/or a GM-CSF molecule can be constructed by chemical synthesis using an oligonucleotide synthesizer. In other aspects, a polynucleotide (e.g., an mRNA) disclosed herein encoding an IL-2 molecule and/or a GM-CSF molecule is made by using a host cell. In certain aspects, a polynucleotide (e.g., an mRNA) disclosed herein encoding an IL-2 molecule and/or a GM-CSF molecule is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding an IL-2 molecule and/or a GM-CSF molecule. The resultant mRNAs can then be examined for their ability to produce protein and/or produce a therapeutic outcome.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

### Purification

In other aspects, a polynucleotide (e.g., an mRNA) disclosed herein encoding an IL-2 molecule and/or a GM-CSF molecule can be purified. Purification of the polynucleotides (e.g., mRNA) encoding an IL-2 molecule and/or a GM-CSF molecule described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding an IL-2 molecule and/or a GM-CSF molecule of the disclosure removes impurities that can reduce or remove an unwanted immune response, e.g., reducing cytokine activity.

In some embodiments, the polynucleotide (e.g., mRNA) encoding an IL-2 molecule and/or a GM-CSF molecule of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes an IL-2 molecule and/or a GM-CSF molecule disclosed herein increases expression of the an IL-2 molecule and/or a GM-CSF molecule compared to polynucleotides encoding the IL-2 molecule and/or GM-CSF molecule purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes an IL-2 molecule and/or a GM-CSF molecule. In some embodiments, the purified polynucleotide encodes a human IL-2 molecule and/or a human GM-CSF molecule.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an RNA nucleic acid of the disclosure comprises N1-methyl-pseudouridine (m1ψ) substitutions at one or more or all uridine positions of the nucleic acid.

In some embodiments, an RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises pseudouridine (ψ) substitutions at one or more or all uridine positions of the nucleic acid.

In some embodiments, an RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above. In some embodiments, the nucleic acid is an otherwise identical nucleic acid that does not comprise one or more or fully modified N1-methylpseudouridine.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the LNP compositions disclosed herein, e.g., an LNP composition comprising a first polynucleotide comprising an mRNA encoding an IL-2 molecule, and/or a second polynucleotide comprising an mRNA encoding a GM-CSF molecule. The present disclosure also provides pharmaceutical formulations comprising an LNP composition comprising a third polynucleotide comprising an mRNA encoding a GM-CSF molecule.

In some embodiments of the disclosure, the polynucleotide are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, or implanted reservoir intramuscular, subcutaneous, or intradermal delivery. In other embodiments, the polynucleotide is formulated for subcutaneous or intravenous delivery. In certain embodiments, the polynucleotide is formulated for subcutaneous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations

The polynucleotide comprising an mRNA encoding an IL-2 molecule and/or a GM-CSF molecule of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: HSA-IL-2 Muteins have a Greater Differential Activation of Tregs Vs Tcon and NK Cells

The ability of wild type IL-2 and IL-2 muteins fused to human serum albumin (HSA) to selectively stimulate signaling in regulatory T cells (Tregs) was evaluated in vitro. Briefly, human peripheral blood mononuclear cells (PBMCs) were stimulated for 30 minutes at 37° C. with supernatant of HeLa cells transfected with mRNA encoding the wild type HSA-IL-2 fusion protein HSA-IL2, or mRNA encoding the HSA-IL-2 mutein fusion proteins HSA-IL2 N88D, HSA-IL2 V69A/Q74P/N88D, HSA-IL2 V69A/Q74PN91K, or HSA-IL2 V91K. Human PBMCs stimulated with a range of concentrations of recombinant IL-2 (rIL-2) was used as a comparator. Stimulation of subsets of cells within the PBMC pool was determined by measurement of STAT5 phosphorylation in Tregs (Foxp3+ CD45RAlow) and in other cell subsets (Foxp3-CD45RAlow non-Treg T cells, CD56hi NK cells, CD56lo NK cells, and NKT cells)

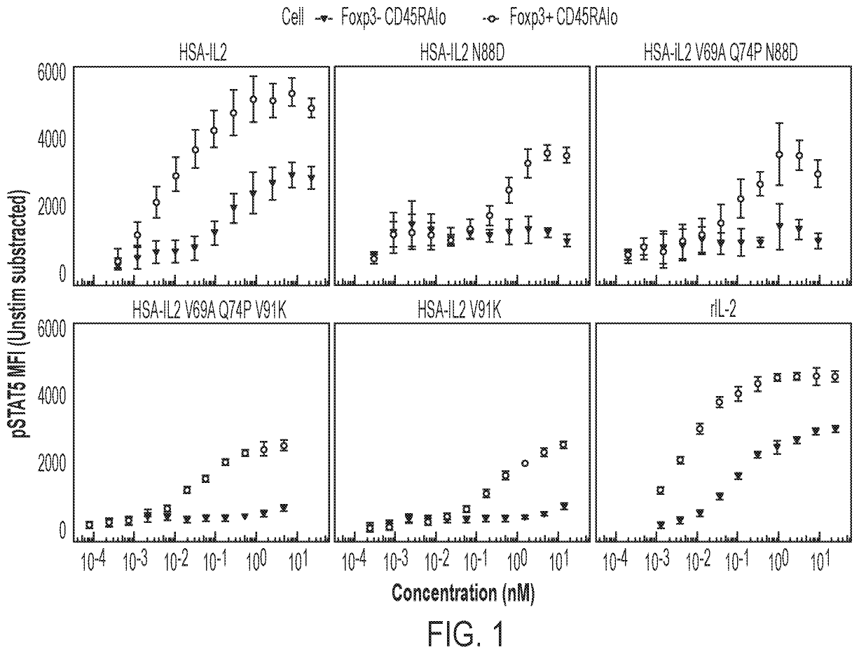

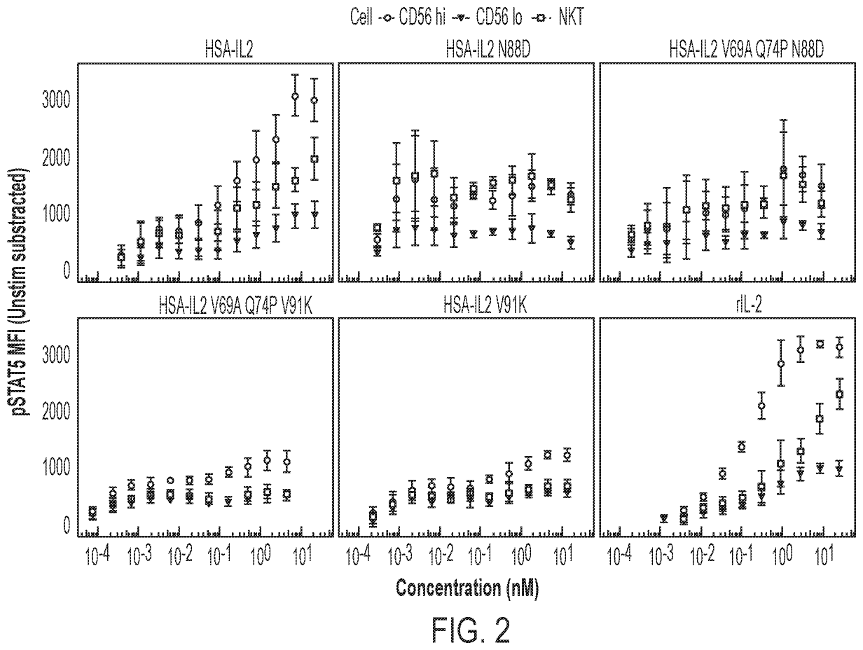

HSA-IL2 fusion protein translated from the mRNA described above showed comparable phosphorylation of STAT5 in T cells (FIG. 1) and NK cells (FIG. 2). The HSA-IL2 mutein fusion proteins translated from the mRNAs described above all showed a reduced EC₅₀ and a reduced maximum pSTAT5 signal in both T cells and NK cells. However, the difference in the pSTAT5 signal indicting stimulation between Tregs and other cell subsets is highest for the 2 triple mutant HSA-IL2 mutein fusion proteins HSA-IL2 V69A/Q74P/N88D and HSA-IL2 V69A/Q74PN91K (FIG. 1 and FIG. 2).

### Example 2: Subcutaneous Administration of LNP-Formulated mRNA Encoding IL-2 Fusion Proteins Expands Tregs in Mice

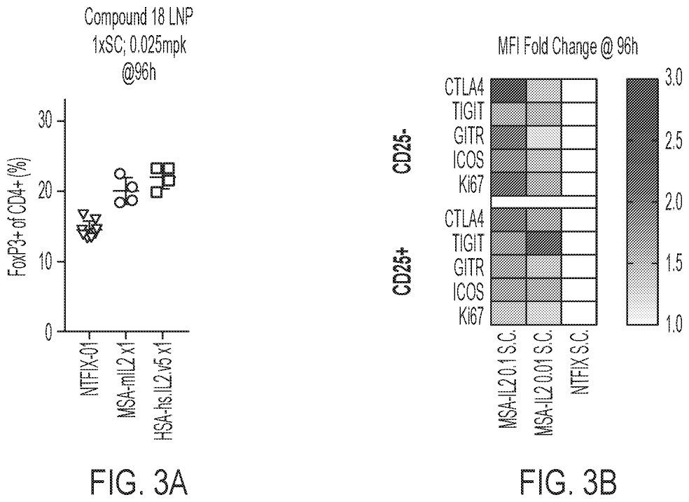

The ability of wild type IL-2 fused to serum albumin to activate and expand regulatory T cells (Tregs) was evaluated in mice. Briefly, mRNA encoding either mouse serum albumin (MSA) fused to mouse wild type IL-2 (MSA-mIL2) or human serum albumin (HSA) fused to human wild type IL-2 (HSA-hs.IL2.v5) was formulated into a lipid nanoparticle (LNP). Mice were treated with a single 0.025 mg/kg dose of the LNP-formulated mRNA administered subcutaneously (SC) and the percentage of CD4+ FoxP3+ Treg cells from serum of treated mice was determined 96 hrs. post-injection (FIG. 3A). Further, the expression level of various Treg activation markers (CTLA4, TIGIT, GITR, ICOS and Ki67) in CD4+ FoxP3+ CD25− and CD25+ Treg cells from serum of treated mice was determined 96 hrs. following SC administration of MSA-IL2 at 0.0025 mg/kg (0.1 S.C.) or 0.00025 mg/kg (0.01 S.C.) (FIG. 3B). LNP-formulated mRNA encoding NTFIX-01 was used as a comparator.

As shown in FIG. 3A, administration of LNP-formulated mRNA encoding MSA-IL2 or HSA-IL2 yielded similar Treg expansion in mice. Furthermore, the Tregs showed an activated phenotype with increased expression of CTLA4, TIGIT, ICOS and GITR (FIG. 3B). These results demonstrate the ability of LNP-formulated mRNA encoding MSA-IL2 or HSA-IL2 to activate and expand Tregs in vivo.

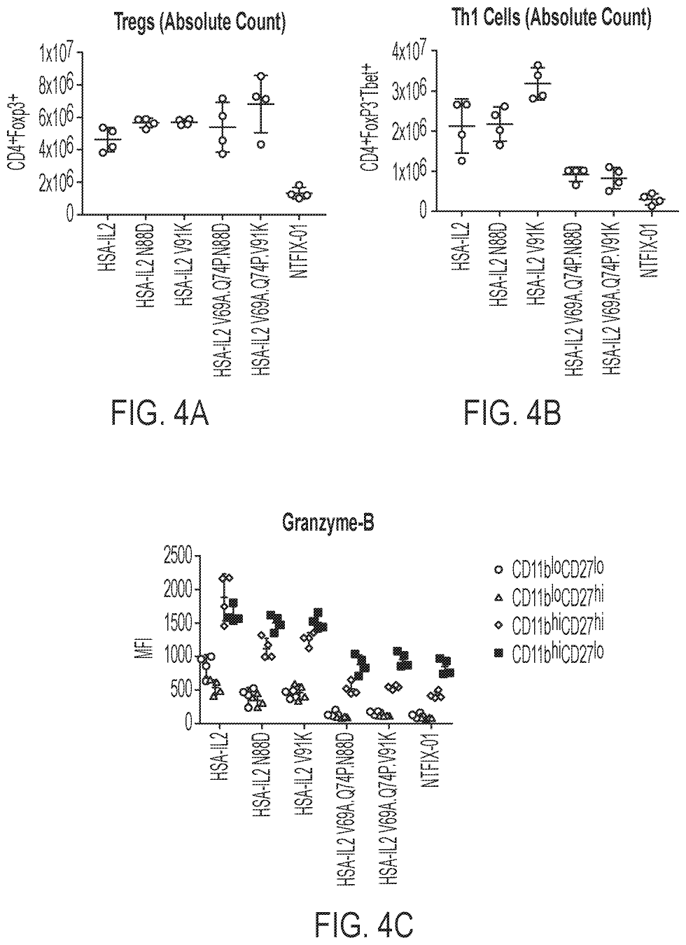

The ability of mRNAs encoding HSA-IL-2 mutein fusion proteins to activate and expand regulatory T cells (Tregs) was also evaluated in mice. Briefly, mRNA encoding the HSA-IL-2 fusion protein HSA-IL2, or mRNA encoding the HSA-IL-2 mutein fusion proteins HSA-IL2 N88D, HSA-IL2 V91K, HSA-IL2 V69A/Q74P/N88D, or HSA-IL2 V69A/Q74PN91K as described in Example 1 were formulated into lipid nanoparticles (LNPs). Mice were independently treated with a single 0.1 mg/kg dose of the LNP-formulated mRNAs administered subcutaneously (SC) and the number of CD4+ FoxP3+ Treg cells (FIG. 4A) and CD4+ FoxP3-Tbet+ Th1 cells (FIG. 4B) from the serum of treated mice was determined 4 days post-injection. Further, the activation of 4 distinct populations of NK cells (CD11b^{lo} CD27^{lo}, CD11b^{lo} CD27^{hi}, CD11b^{hi} CD27^{hi}, and CD11b^{hi} CD27^{lo}) from the serum of treated mice was determined by measurement of Granzyme-b expression (FIG. 4C). LNP-formulated mRNA encoding NTFIX-01 was used as a comparator.

Treatment of mice with LNP-formulated mRNA encoding any one of the four human IL-2 mutein fusion proteins (HSA-IL2 N88D, HSA-IL2 V91K, HSA-IL2 V69A/Q74P/N88D, or HSA-IL2 V69A/Q74PN91K) led to expansion of Tregs 4 days after dosing, with comparable increased of total number of Tregs as to the wild type HSA-IL2 fusion protein (FIG. 4A). Treatment of mice with the wild type HSA-IL2 fusion protein or either of the single mutant HSA-IL2 N88D and HSA-IL2 V91K mutein fusion proteins also resulted in the expansion of Th1 cells (FIG. 4B) and the activation of NK cells (FIG. 4C). In contrast, the triple mutant HSA-IL2 V69A/Q74P/N88D or HSA-IL2 V69A/Q74PN91K mutein fusion proteins showed no increase in Granzyme-B and significantly reduced expansion of Th1 cells (FIG. 4B and FIG. 4C) relative to wild type HSA-IL2 or the single mutant HSA-IL2 N88D and HSA-IL2 V91K mutein fusion proteins. These results demonstrate that treatment of mice with LNP-formulated mRNAs encoding the wild type HSA-IL2 fusion protein and the HSA-IL2 mutein fusion proteins HSA-IL2 N88D, HSA-IL2 V91K, HSA-IL2 V69A/Q74P/N88D, or HSA-IL2 V69A/Q74PN91K expand Tregs. Furthermore, these results shown that the triple mutant fusion proteins HSA-IL2 V69A/Q74P/N88D and HSA-IL2 V69A/Q74PN91K do not appreciably activate Th1 cells or NK cells, indicating the selective expansion of Tregs in vivo.

### Example 3: Subcutaneous Administration of LNP-Formulated mRNA Encoding IL-2 Fusion Proteins Expands Tregs in Cynomolgus Monkeys

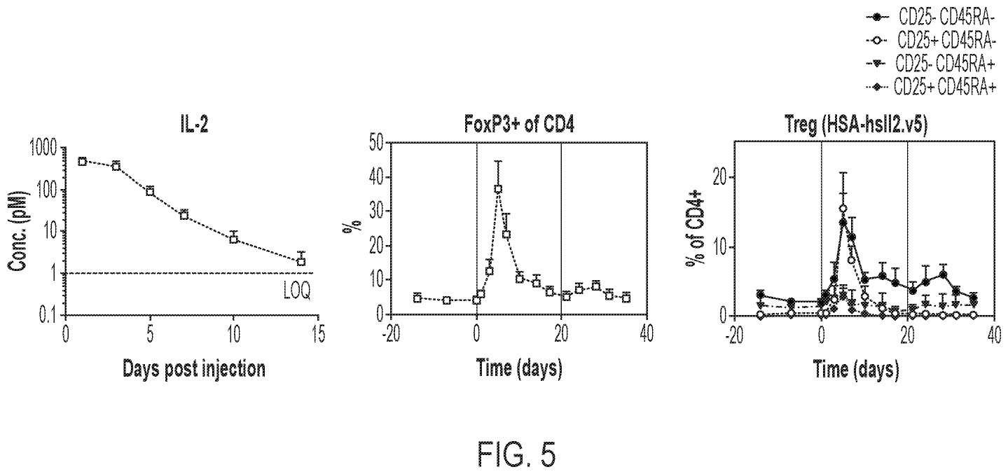

The ability of wild type human IL-2 fused to human serum albumin (HSA) to expand regulatory T cells (Tregs) was evaluated in cynomolgus monkeys. Briefly, monkeys were treated by subcutaneous administration of a single dose of LNP-formulated mRNA encoding HSA-hs.IL2.v5 described in Example 2 by subcutaneous administration. The concentration (in pM) of the HSA-hs.IL2.v5 polypeptide in serum of treated monkeys was determined over time (FIG. 5A). The percentage of CD4+ FoxP3+ Treg cells (FIG. 5B) and Treg cell subsets (CD25-CD45RA−, CD25+ CD45RA−, CD25− CD45RA+, and CD25+ CD45RA+; FIG. 5C) from serum of treated monkeys was determined pre- and post-injection.

As shown in FIG. 5A, the HSA-IL2 fusion protein reached a peak concentration of 500 pM at 24 hrs. post-injection and clearance half-life of 24 was observed. As shown in FIG. 5B, the percentage of Treg (CD4+ FoxP3+) cells within the CD4+ cell population was elevated for a two-week window, returning to baseline 20 days after administration. Further, as shown in FIG. 5C, a subset of Tregs (CD25− CD45RA−) remained elevated 4 weeks following administration. These results demonstrate that treatment of cynomolgus monkeys with LNP-formulated mRNAs encoding the wild type HSA-IL2 fusion protein HSA-hs.IL2.v5 results in the expansion of Tregs and that a subset of Tregs (CD25− CD45RA−) remain elevated for 4 weeks following treatment.

### Example 4: MSA-IL2 is Protective in a Model of Acute Graft Vs. Host Disease

This Example describes the effect of administration of LNPs comprising an mRNA encoding MSA-IL2 in a model of acute graft vs. host disease (GvHD).

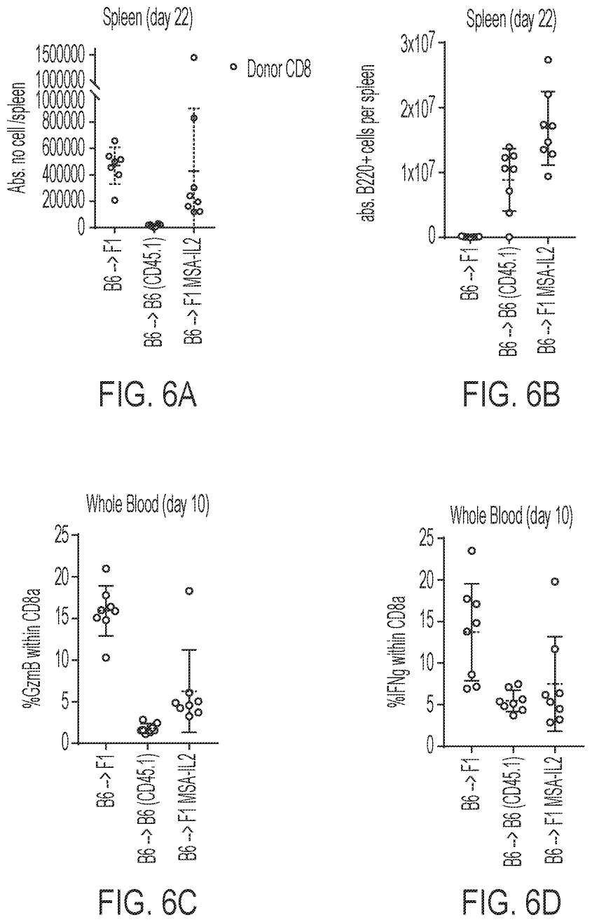

The experimental design is depicted in FIG. 6A. Fifty million splenocytes plus 5 million CD4+ T cells from a C57BL/6 mice donor (B6) were transferred to the progeny of B6 crossed with DBA mice (F1) to result in a partial mismatch. Animals were dosed on day 1, 8 and 15 with lipid nanoparticle-formulated mRNA encoding MSA-IL2.

The data shows that weekly administration of an mRNA encoding MSA-IL2 at 0.025 mpk formulated in LNPs comprising Compound 18 as the ionizable lipid, led to reduced donor CD8 and CD4 T cell engraftment. Furthermore, the host B cell population was retained (FIG. 6B) and the T cells isolated from peripheral blood had reduced CTL responses (FIG. 6C and FIG. 6D).

### Example 5: RSA-IL2 Reduces Severity of Arthritis in the Rat Collagen-Induced Arthritis Model

This Example describes the effect of administration of LNPs comprising an mRNA encoding RSA-IL2 in a rat model of collagen-induced arthritis.

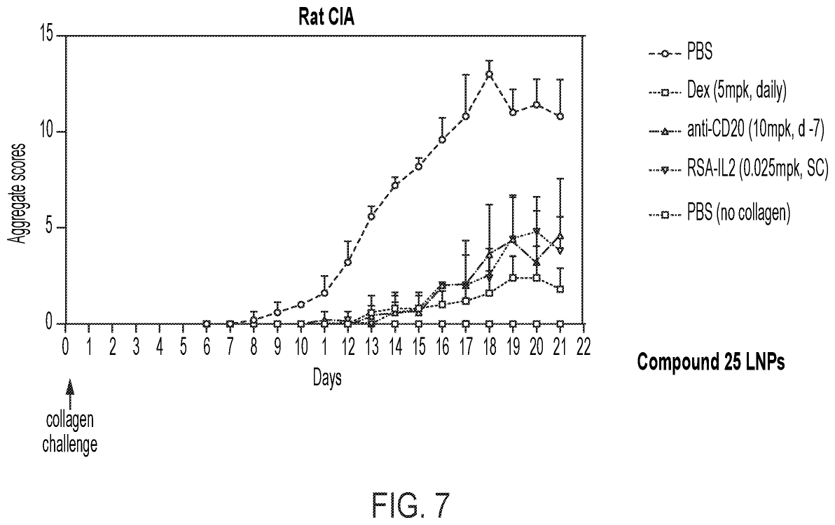

Weekly intramuscular administration of an mRNA encoding RSA-IL2 at 0.025 mpk formulated in LNPs comprising Compound 25 as the ionizable lipid, led to a reduction in the aggregate score of arthritis in a collagen-induced rat arthritis model (FIG. 7).

### Example 6: IL-2 Fusion to Targeting Domains

To increase the specificity of IL-2 towards stimulation of Tregs, a fusion of IL-2 to a ligand of or to an antibody targeting a polypeptide that is overexpressed or selectively expressed on Tregs is contemplated. One such example is the fusion of HSA-IL2 to a single domain antibody against CTLA4, which is constitutively expressed at high levels on Tregs. Furthermore, other targeting moieties are contemplated for tissue specific retention for example. With selection of the affinities for the targeting receptor and the modification of IL-2 affinity, increased selectivity for the target tissue or cell type is contemplated.

### Example 7: GM-CSF Fusion to Serum Albumin Allows Reduced Dosing Levels and Frequency for Treg Induction

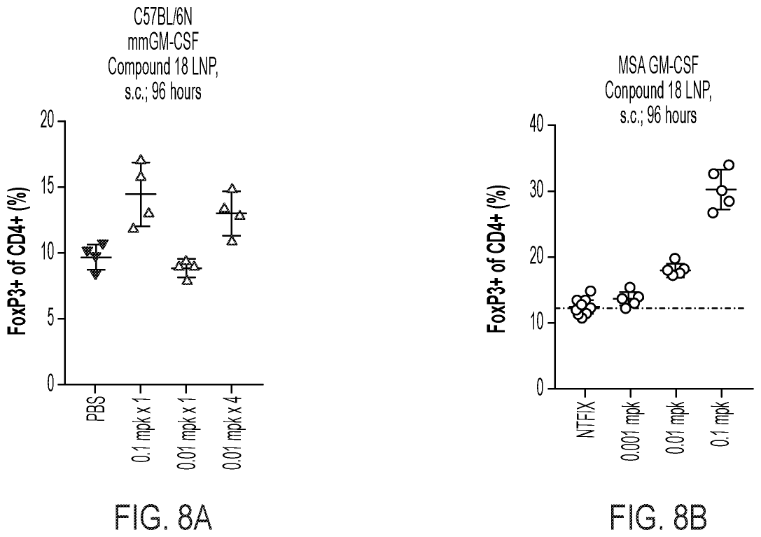

Wild-type GM-CSF mRNA formulated LNP (comprising Compound 18) injection in mice led to an increase in the frequency of Tregs within the CD4+ compartment, when administered as a single dose at 0.1 mg/kg. At lower doses, the effect is lost (FIG. 8A). Next, the effect of an mRNA encoding a GM-CSF-serum albumin fusion protein was tested. The mRNA encoding a GM-CSF-serum albumin fusion protein was formulated in an LNP formulation and injected subcutaneously in mice. As shown in FIG. 8B, LNP formulated mRNA encoding a GM-CSF-serum albumin fusion protein can increase the Treg frequency at levels as <0.01 mpk.

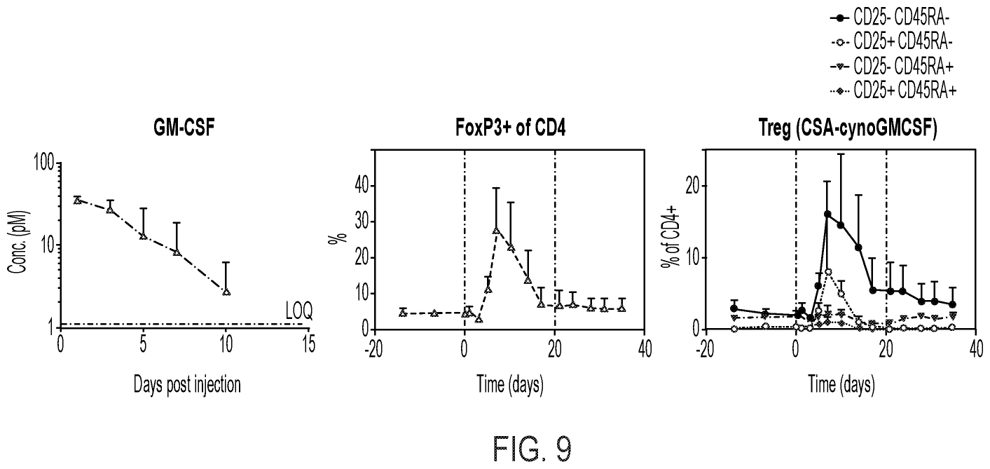

In cynomolgus monkey, a similar effect was observed. After subcutaneous administration of an mRNA encoding GM-CSF-serum albumin fusion protein formulated in an LNP, the cyno serum albumin fusion to GM-CSF was detectable in serum for up to 10 days and the frequency of Tregs was elevated during a two-week window (FIG. 9).

### Example 8: Combination of IL-2 and GM-CSF Improves Selective Expansion of Tregs

It was previously observed that IL-2 led to the expansion of Th1 and Th2 cells in addition to the expansion of Tregs. It was also observed that GM-CSF led to the expansion of Th17 cells. Additionally, it is known that IL-2 can suppress the Th17 differentiation pathway.

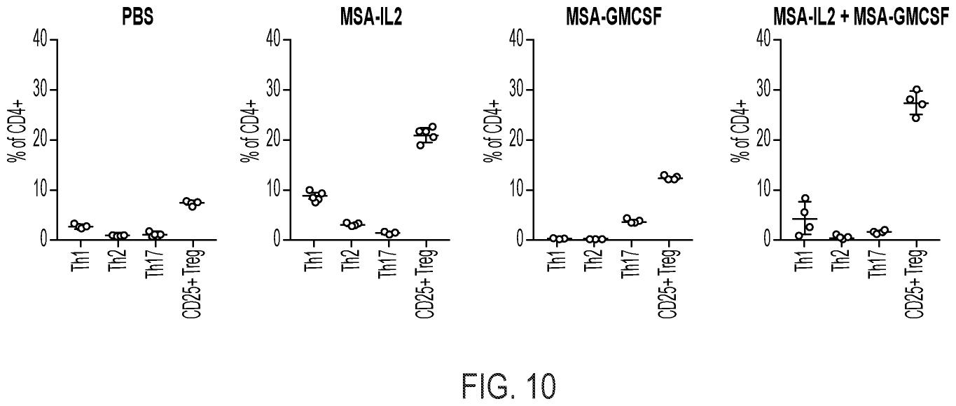

The combination of serum albumin fusion of IL-2 and GM-CSF to induce Tregs but suppress the Th17 expansion observed with GM-CSF was next evaluated. Mice were administered LNPs comprising mRNAs encoding MSA-IL2, MSA-GMCSF or MSA-IL2 and MSA-GMCSF. The LNP formulations were administered at 0.1 mg per kg. As shown in FIG. 10, 96 hours post administration, the LNP formulation comprising MSA-IL2 and MSA-GMCSF did not result in an increase in the fraction of Th17 cells in the CD4 T cell population. A reduction in the IL-2 induced Th1 and Th2 expansion was also observed in mice administered the LNP formulation comprising MSA-IL2 and MSA-GMCSF (FIG. 10).

Given that GM-CSF and IL-2 have different mechanism of action and that the effects of GM-CSF are generally delayed due to the recruitment of myeloid progenitor cells, the effects of combinations of GM-CSF and IL-2 under different treatment schedules was evaluated. Animals were administered MSA-IL2 containing LNPs, MSA-GMCSF containing LNPs, MSA-IL2 and MSA-GMCSF containing LNPs (“combo”), or a sequential administration of MSA-GMCSF containing LNP followed by MSA-IL2 and MSA-GMCSF containing LNPs.

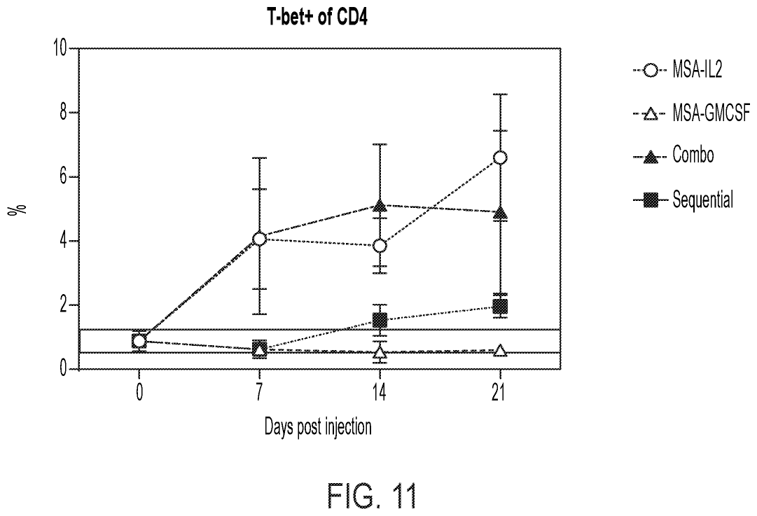

FIG. 11 shows the fraction of T-bet+ CD4 T cells (Th1) over a 4-week window with weekly treatment of MSA-IL2 and/or MSA-GMCSF subcutaneously in an LNP formulation. MSA-IL2 led to gradual increased in Th1 cell frequency. A similar increase in Th1 cell frequency was observed for the combination of MSA-IL2 and MSA-GMCSF (“combo”) at this specific dose, route of administration and evaluation time point. Under the sequential schedule, the animals were first dosed with GM-CSF and then subsequent doses were a combination of GM-CSF and IL2. Therefore, the animals in the sequential day 14 received the same total dose of IL-2 as the combo day 7. The same is applicable for day 21 and day 14 respectively.

As shown in FIG. 11, the sequential dosing schedule resulted in a reduced increase of Th1 cell frequency as compared to the Th1 cell frequencies in MSA-IL2 or combo dosing schedules.

This data suggests that a specific dosing schedule and dosing ratio of the combination of IL-2 and GM-CSF can be determined, to reduce the pro-inflammatory effects associated with these cytokines (Th1, Th2, Th17) and promote the suppressive effects (Tregs).

### Example 9: Expansion and Activation of T Regulatory Cells with LNP Formulated HSA-IL2

This example describes expansion and activation of T regulatory cells in primates dosed with HSA-IL2.

To evaluate the effect of in vivo dosing of LNP formulated HSA-IL2 mRNA in primates, male cynomolgus monkeys (4 per group) were dosed in scapular and dorsal areas on days 1, 29, 43 and 57. The animals were dosed subcutaneously with LNP formulated HSA-IL2 at a dose of 0.01 mg per kg, 0.03 mg per kg or 0.1 mg per kg. All animals were dosed at 1 ml/kg.

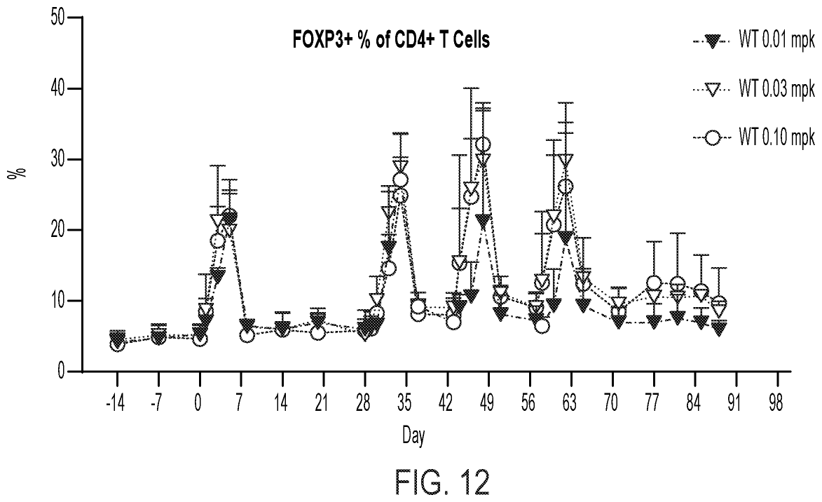

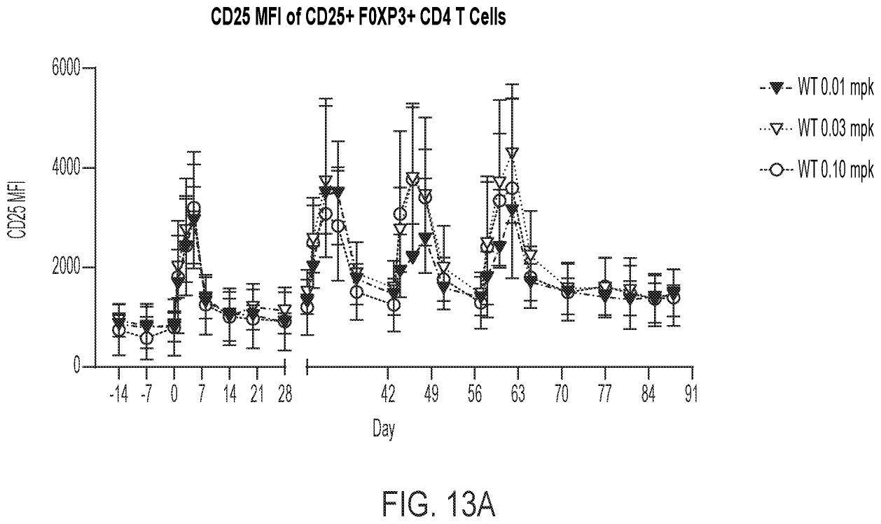

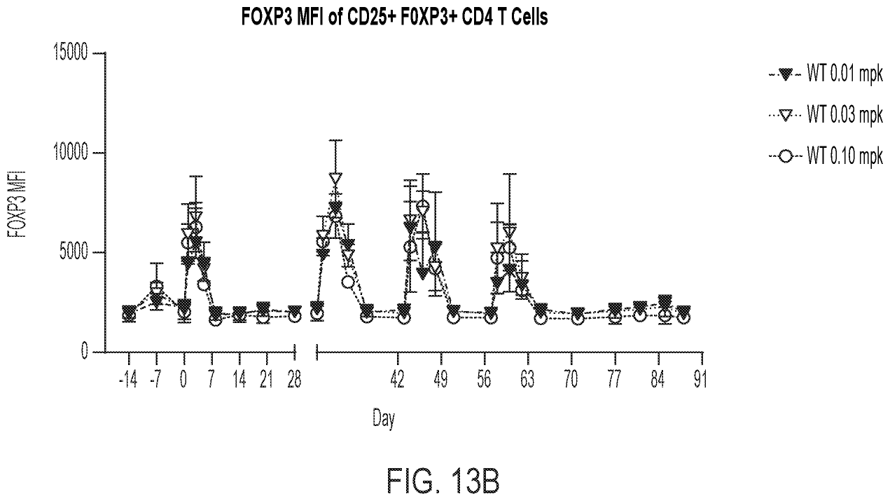

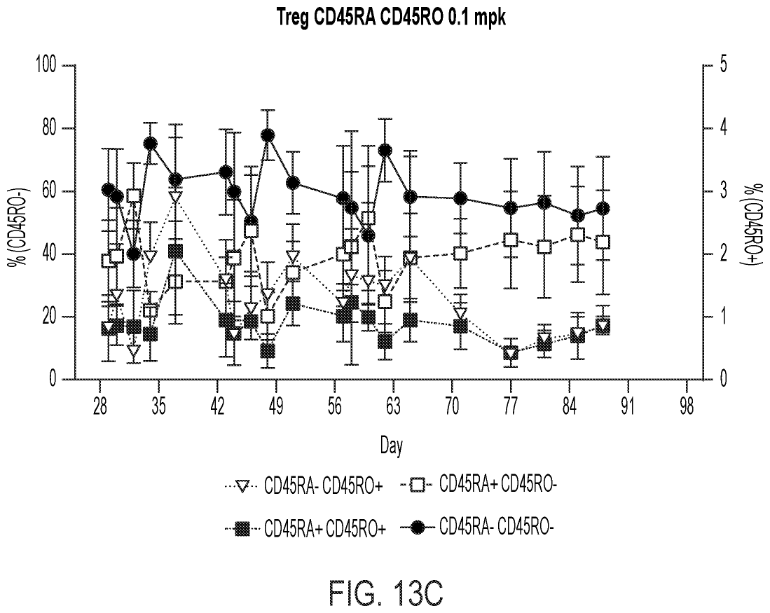

Administration of LNP formulated HSA-IL2 mRNA resulted in reproducible Treg expansion with each LNP formulated HSA-IL2 mRNA dose (FIG. 12). The observed response was at its maximum at the lowest dose of 0.01 mg per kg (FIG. 12). The T regulatory cells from the LNP dosed animals had an activated phenotype with increased CD25 and FOXP3 (FIGS. 13A-13C).

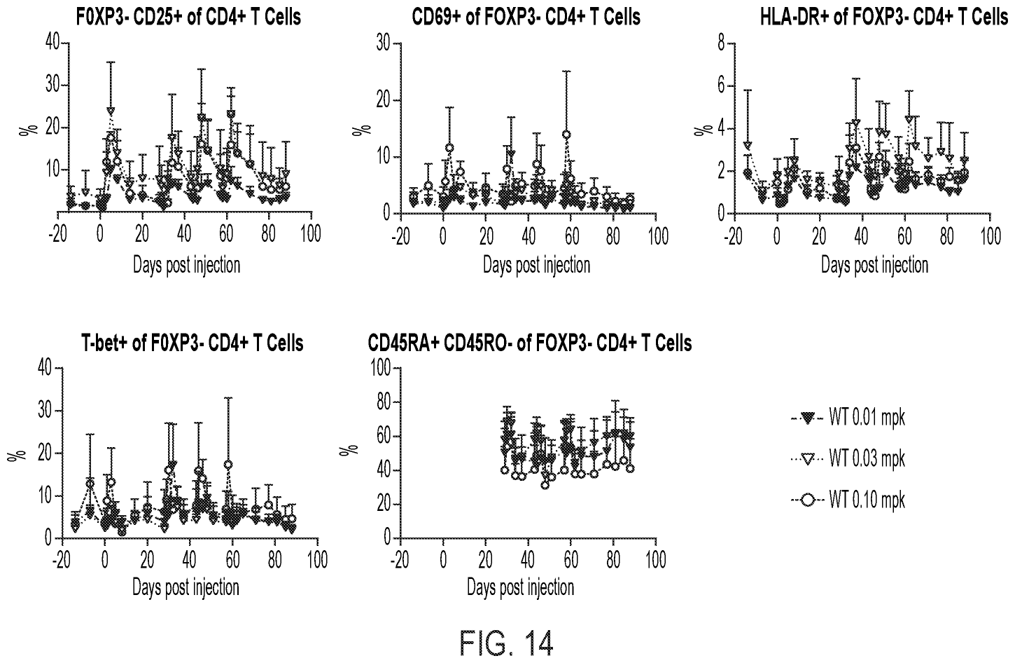

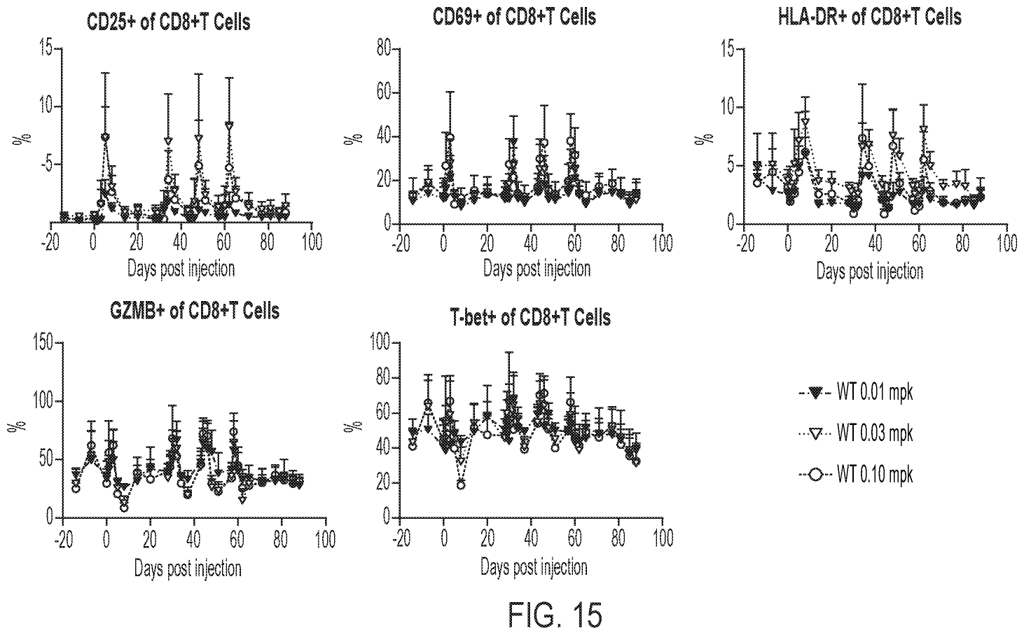

With respect to T conventional cells (T con), activation of this population of cells was observed at 0.03 mg per kg and 0.10 mg per kg, with reduced activation at the 0.01 mg per kg dose (FIG. 14). CD8 T cells showed transient activation, with maximal activation observed at 0.03 mg per kg of LNP formulated HSA-IL2 (FIG. 15). Taken together the data described in this example shows that LNP formulated HSA-IL2 results in in vivo expansion and activation of T regulatory cells.

### Example 10: Induction of Plasma Cytokines In Vivo with Administration of LNP Formulated HSA-IL2

This Example describes induction of plasma cytokines in primates administered LNP formulated HSA-IL2. A similar experimental setup as described in Example 9 was used in this example.

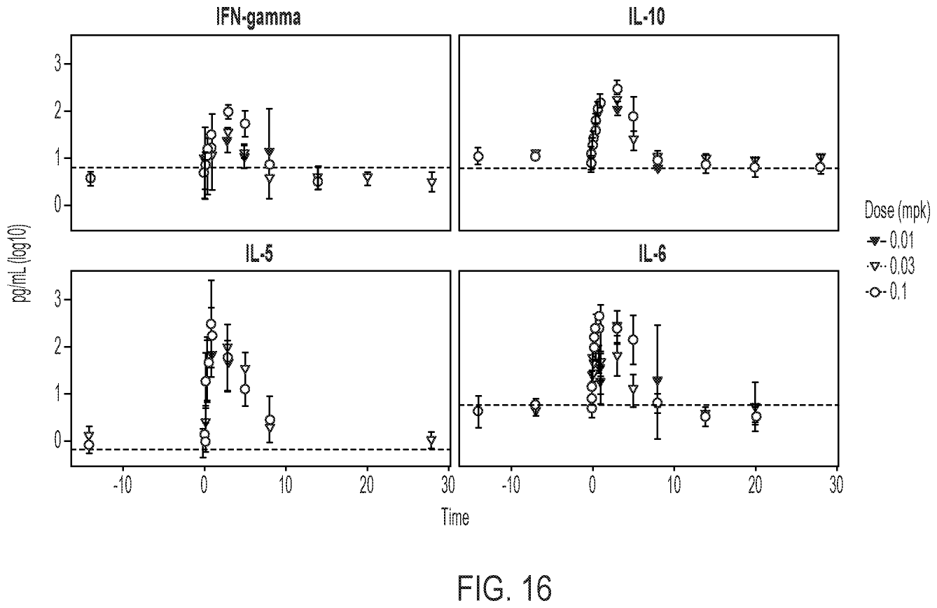

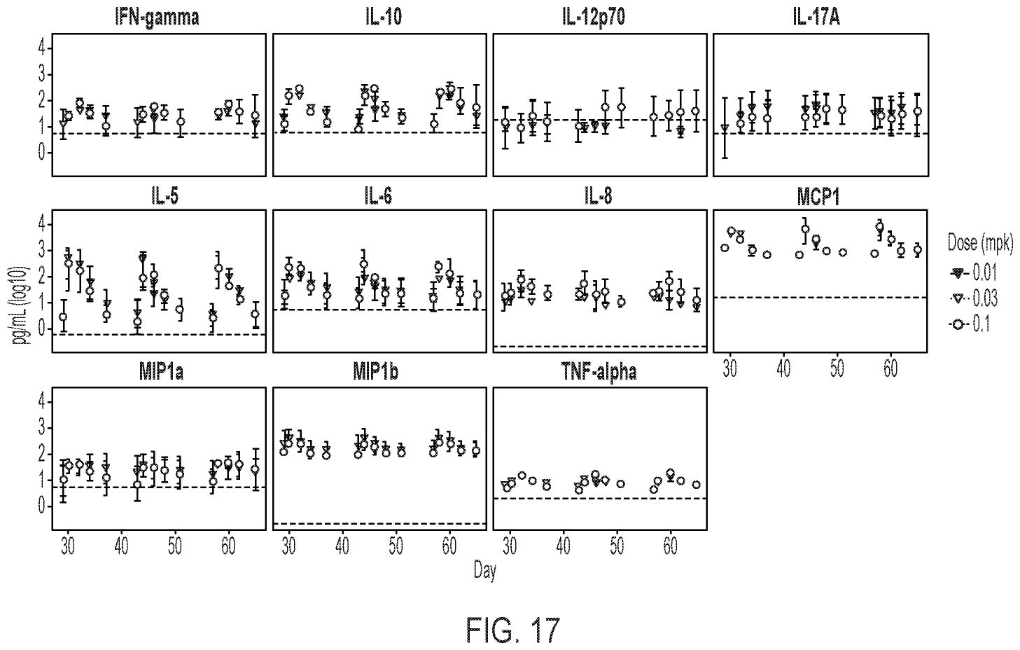

Administration of LNP formulated HSA-IL2 resulted in elevation of Th1 and Th2 cytokines (FIG. 16), as well as several other cytokines including IFNγ, IL-10, IL-5, IL-6, MCP-1, MIP1b and TNFα (FIG. 17). The data shows that LNP formulated HSA-IL2 mRNA results in in vivo induction of cytokines.

### Example 11: Preferential Expansion of Treg Over Tcon in Non-Human Primates

This Example describes the preferential expansion of T regulatory cells (CD4+ FoxP3+) as compared to activated CD8+ T conventional (T con) cells (which co-express CD25) in non-human primates administered LNP formulated HSA-IL2 mRNA. The experimental setup used in this study was similar to that used in Example 9. The animals were administered either LNP formulated wild type HSA-IL2 mRNA or LNP formulated variant HSA-IL2 (N88D; also referred to as TM88 herein) mRNA.

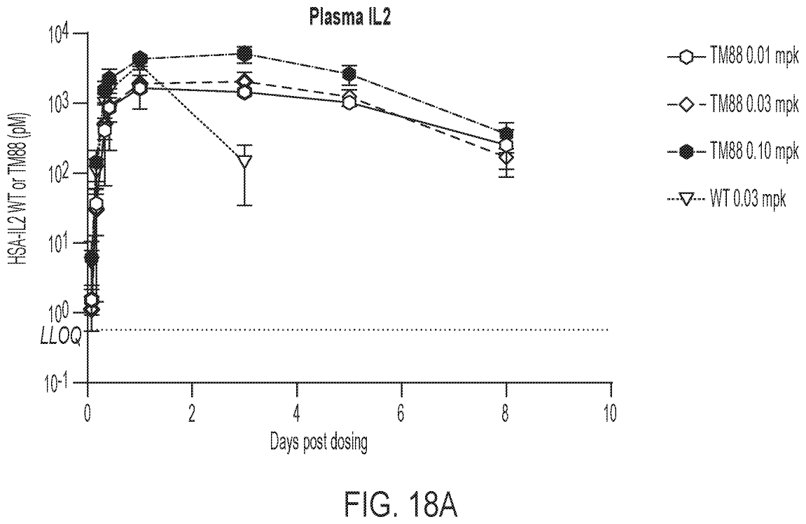

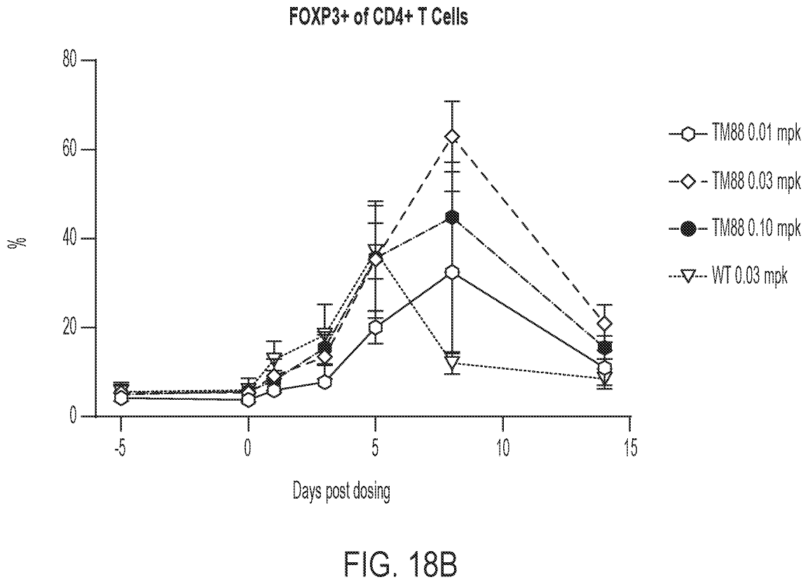

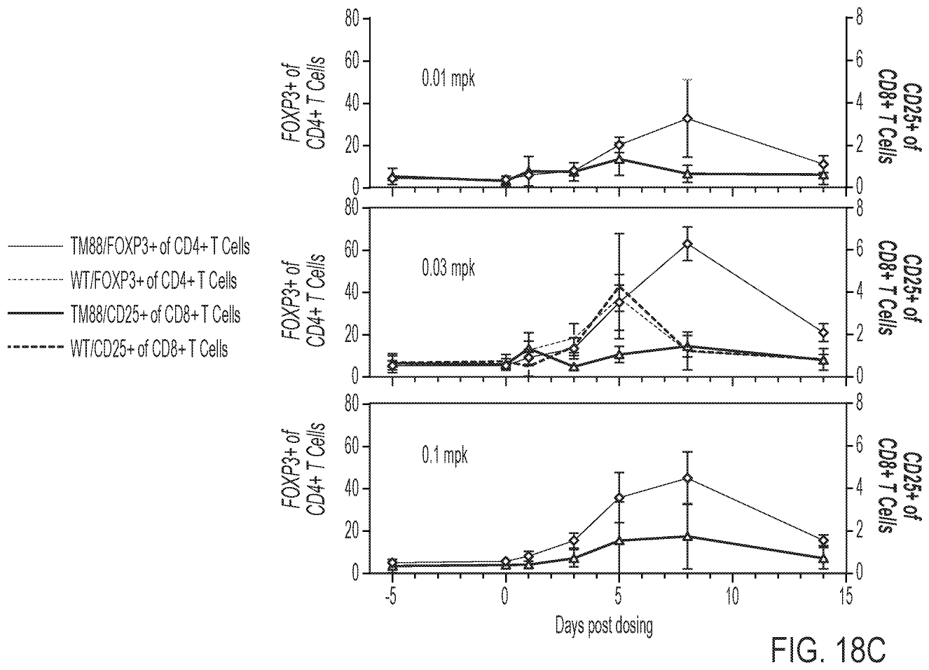

The TM88 IL2 variant conjugated to HSA formulated in an LNP, demonstrated a longer half-life in vivo as compared to wild type IL2 conjugated to HSA formulated in an LNP (FIG. 18A). This increased half-life is due to, e.g., reduced IL2R mediated degradation. In addition, LNP formulated HSA-IL2 TM88 showed proliferation of T regulatory cells for about 10 days post dosing, with maximal proliferation observed at about day 7 post dosing (FIG. 18B). In contrast, LNP formulated HSA-IL2 showed maximal T regulatory proliferation at about day 5 post dosing. Two weeks after dosing, the frequency of Tregs was still elevated (up to 4× with 0.03 mg per kg) with LNP formulated HSA-IL2 TM88 compared to LNP formulated HSA-IL2 mRNA, indicating a prolonged proliferation of Tregs.

As compared to administration of LNP formulated HSA-IL2 mRNA, administration of LNP formulated HSA-IL2 TM88 resulted in greater and longer increase of FOXP3 expression level (FOXP3 MFI) and CD25 expression level (CD25 MFI) in CD25+ Foxp3+ CD4 T cells, indicating that LNP formulated HSA-IL2 TM88 mRNA further activated Tregs compared to LNP formulated HSA-IL2 mRNA.

The effect of LNP formulated HSA-IL2 TM88 mRNA on T regulatory cell expansion was further characterized. FIG. 18C shows the preferential expansion of T regulatory cells (CD4+ FoxP3+) as compared to activated CD8+ T conventional (T con) cells (which co-express CD25) in non-human primates administered LNP formulated HSA-IL2 at all doses tested. The data shows that LNP formulated HSA-IL2 TM88 mRNA shows greater selectivity of Treg vs CD8 expansion and/or activation. In addition, while the data show that LNP formulated wildtype HSA-IL2 activated T con cells early (i.e., as early as about 3 days post injection; 0.03 mg per kg), activation of T con cells after LNP formulated HSA-IL2 TM88 mRNA administration (0.03 mg per kg and 0.1 mg per kg) was not observed.

As noted in Example 10, administration of LNP formulated HSA-IL2 mRNA resulted in in vivo induction of several cytokines. In comparison to administration of LNP formulated HSA-IL2, the data shows such elevation in IL-5, IL-6, TNFα, INFγ, and IL-10 was reduced where the LNP administered was formulated HSA-IL2 TM88. The reduced production of IFNγ and IL-5 indicates a greater specificity in reducing Th1 and Th2 differentation by LNP formulated HSA-IL2 TM88.

### Example 12: Characterization of Duration of Treg Expansion in Non-Human Primates

This example describes the phenotypic characterization of T regulatory cell activation in primates dosed with LNP formulated with HSA-IL2 TM88 mRNA.

To evaluate the effect of in vivo dosing of LNP formulated HSA-IL2 TM88 in primates, male cynomolgus monkeys (4 per group) were dosed on days 1, 15, and 29. The animals were dosed subcutaneously with LNP formulated HSA-IL2 TM88 mRNA at a dose of 0.001 mg per kg, 0.003 mg per kg, 0.006 mg per kg, or 0.01 mg per kg. All animals were dosed at 1 ml/kg.

The T regulatory cells from the LNP dosed animals had an activated phenotype with increased CD25 and FOXP3. While variability of the extent of activation was observed across animals, administration of LNP formulated HSA-IL2 TM88 resulted in T regulatory cell proliferation at all dose levels with a maximal proliferation at about 8 days post injection. It was reported that Tregs levels pearked at 4 days post injection when Fc-IL2N88D was administered in cynomolgus under conditions tested (Bell et al. in the Journal of Autoimmunity, 56 2015, 66-80). The majority of the animals exhibited consistent Treg expansion following each dose, with only three subjects exhibiting a shortened expansion following the third dose.

Other activation markers further support maximal proliferation of T regulatory cells between 8 and 11 days post injection. Specifically, increase in Ki67 proliferation was observed prior to Treg expansion, with maximal activation at 8 days post injection, and CD25 upregulation persists beyond the maximal Treg expansion at 8 days post injection.

At dosages of 0.006 and 0.01 mg per kg, T regulatory cells exhibited a phenotype consistent with increased proliferation, activation, and suppressive functionality.

One animal also exhibited an increase in CD25 of CD8+ T conventional (T con) cells (CD25+ FOXP3−) after injection; however, other activation markers of CD8+ T cells were not upregulated (CD27+, CD45RA+, CD69+, FOXP3+, PD1+).

### Example 13: Clearance of LNP Formulated HSA-IL2 TM88 in Non-Human Primates

This Example describes the relationship between plasma levels of HSA-IL2 TM88 and T regulatory cell expression in primates administered LNP formulated HSA-IL2 TM88 mRNA. A similar experimental setup as described in Example 12 was used in this example.

Dose-dependence of HSA-IL2 TM88 plasma levels was observed, as higher plasma concentrations of HSA-IL2 TM88 were observed with higher administered doses. Exponential decay of plasma concentration of HSA-IL2 TM88 was observed across all dosage levels with a clearance half-life of about 3.7 days. The data suggest a persistence of the HSA-IL2 TM88 mRNA in vivo, which shows an improvement over an Fc-IL2 N88D mutein, which exhibited shorter half-lives, i.e., 8 and 14 h for intravenous and subcutaneous administrations, respectively (Bell et al. in the Journal of Autoimmunity, 56 2015, 66-80).

A linear correlation between HSA-IL2 TM88 mRNA concentration and T regulatory cell expression at 8 days post injection was observed for HSA-IL2 TM88 mRNA concentrations greater than 1.0 nM at 48 h post injection (i.e. for dosages of 0.003, 0.006, and 0.01 mg per kg). A positive correlation between HSA-IL2 TM88 mRNA concentration and T regulatory cell expression persisted until after 9 days post injection.

### Example 14: Anti-PEG/PC IgG/IgM Response to Administration of LNP Formulated HSA-IL2 TM88 in Non-Human Primates

This Example describes the response of anti-PEG and anti-PC immunoglobins to administration of LNP formulated HSA-IL2 TM88 mRNA in primates. A similar experimental setup as described in Example 12 was used in this example.

Anti-PEG IgM, anti-PC IgM, anti-PEG IgG, and anti-PC IgG concentrations were measured at 1, 15, 29, and 44 days post initial injection. Immunoglobin concentrations remained consistent over time and did not exhibit significant increase with increasing dosages. The data shows that LNP formulated HSA-IL2 TM88 mRNA did not induce a significant antibody response to PEG and PC.

### Example 15: Evaluation of LNP Formulated HSA-IL2TM88 in the MOG35-55 EAE Mouse Model

This Example describes the effect of administration of LNPs comprising HSA-IL2 TM88 in the MOG35-55 experimental autoimmune encephalomyelitis (EAE) mouse model.

Mice (12 per group) were dosed on days −3, 0, 3 and 6 (4×) subcutaneously with LNP formulated HSA-IL2 TM88 mRNA or HSA mRNA negative control at a dose of 0.1 mg per kg, or on days −3 and 0 (2×) subcutaneously with LNP formulated with an mRNA encoding HSA-IL2 TM88 or HSA negative control at a dose of 0.03 mg per kg, or administered on days −3, 0, 3 and 6 subcutaneously with PBS vehicle control. MOG35-55 was dosed on day 0 at a dose of 100 μg per mouse. Complete Freund 9 Adjuvant (CFA) was used on day 0 at a final concentration of 2.5 mg/ml M. tuberculosis. 200 ng per mouse pertussis toxin (PTX) was injected intraperitoneally on days 0 and 2.

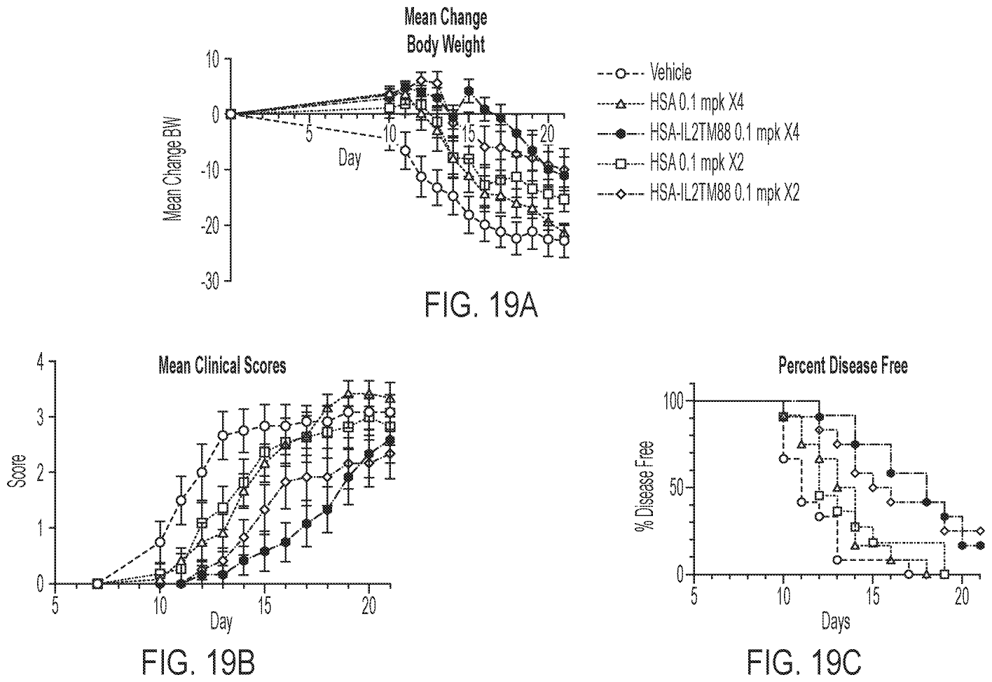

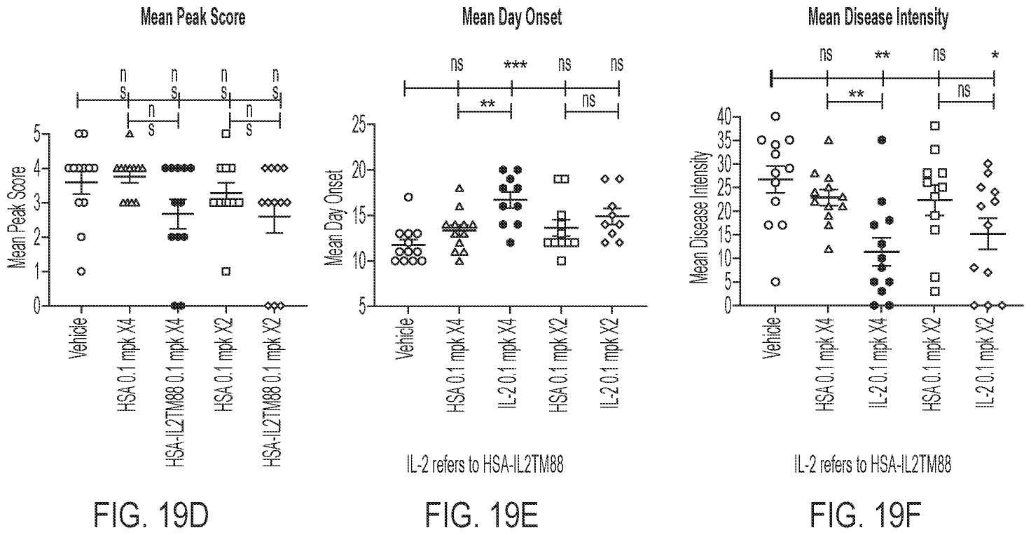

As shown in FIGS. 19A-19F, disease onset was delayed, and progression was slower for animals treated with LNP formulated HSA-IL2 TM88 mRNA. Both dosing paradigms (2× & 4×) of IL2 showed delays in disease onset compared to vehicle controls and HSA controls. The 4× IL2 group showed stronger delay and activity than the 2× IL2 group leading to a dose response in terms of overall total dose. The trend for a delay with the HSA controls relative to vehicle controls was not significant. There was a delay with IL-2 treatment, however this protection abated by day 21. The mean peak scores caught up to controls and were not significantly different in the end. The most significant endpoint affected in this study in the 21-day time frame was disease intensity which was observed for both IL2 groups.

### Example 16: Evaluation of LNP Formulated HSA-hsGM-CSF in Cynomolgus Monkeys

This Example describes the effect of administration of LNPs (comprising Compound 18) comprising an mRNA encoding a human GM-CSF-human serum albumin fusion protein (HSA-hsGM-CSF) (hsGMCSF construct comprising the sequences shown in Table 3B) in cynomolgus monkeys.

Male cynomolgus monkeys (4 per group) were dosed on day 1 subcutaneously with LNP formulated with HSA-hsGM-CSF mRNA at a dose of 0.01 mg per kg, 0.03 mg per kg, or 0.1 mg per kg. All animals were dosed at 1 mL/kg.

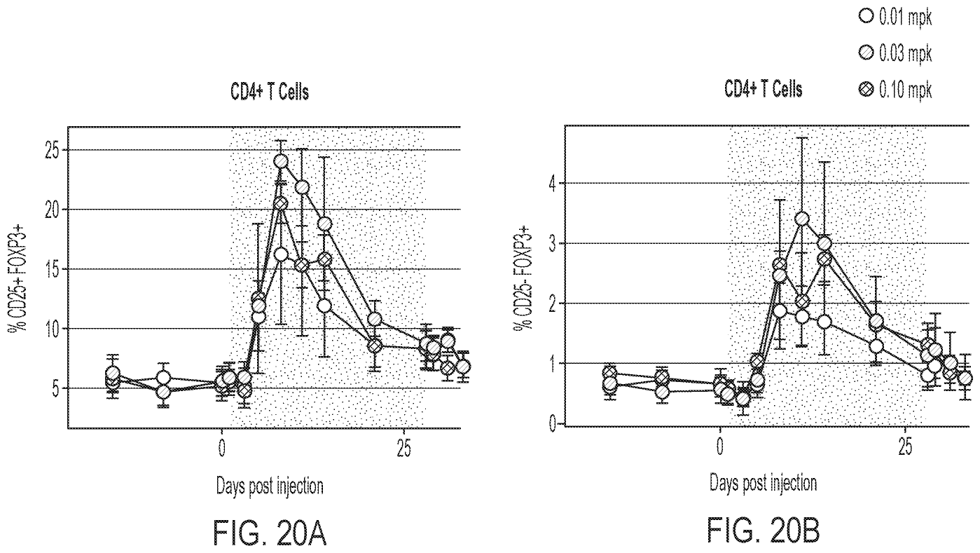

As shown in FIGS. 20A-20B, subcutaneous administration of LNP formulated with an mRNA encoding HSA-hsGM-CSF resulted in CD25− and CD25+ Treg expression in cynomolgus monkeys.

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A lipid nanoparticle (LNP) composition comprising a polynucleotide comprising an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables 1A, 2A or 4A.

2. The LNP composition of claim 1, wherein the IL-2 molecule comprises a naturally occurring IL-2 molecule, a fragment of a naturally occurring IL-2 molecule, or a variant thereof.

3. The LNP composition of claim 1 or 2, wherein the IL-2 molecule comprises a variant of a naturally occurring IL-2 molecule (e.g., an IL-2 variant, e.g., as described herein), or a fragment thereof.

4. The LNP composition of any one of claims 1 to 3, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).

5. The LNP composition of any one of claims 1 to 4, wherein the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.

6. The LNP composition of any one of claims 2 to 5, wherein the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.

7. The LNP composition of any one of claims 2 to 6, wherein the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.

8. The LNP composition of any one of claims 2 to 7, wherein the IL-2 variant comprises a mutation, e.g., substitution, at position 88 of the IL-2 polypeptide sequence, e.g., an N88D substitution.

9. The LNP composition of any one of claims 2 to 8, wherein the IL-2 variant comprises a mutation, e.g., substitution, at position 91 of the IL-2 polypeptide sequence, e.g., a V91K substitution.

10. The LNP composition of any one of claims 2 to 9, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 88 of the IL-2 polypeptide sequence, e.g., an N88D substitution.

11. The LNP composition of any one of claims 2 to 9, wherein the IL-2 variant comprises a mutation, e.g., substitution, at:
position 69 of the IL-2 polypeptide sequence, e.g., a V69A substitution;
position 74 of the IL-2 polypeptide sequence, e.g., a Q74P substitution; and
position 91 of the IL-2 polypeptide sequence, e.g., a V91K substitution.

12. The LNP composition of any one of claims 2 to 11, wherein the IL-2 variant comprises a mutation, e.g., substitution, at position 125 of the IL-2 polypeptide sequence, e.g., a C125S substitution.

13. The LNP composition of any one of claims 1 to 12, wherein the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.

14. The LNP composition of any one of claims 1 to 13, wherein the IL-2 molecule comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.

15. The LNP composition of any one of claims 1 to 14, wherein the polynucleotide encoding the IL-2 molecule comprises a nucleotide sequence having at least 85%, 90%, 95%, 96%, 97%, 98%, 99% or 100% identity to the sequence of SEQ ID NO: 7, SEQ ID NO: 25 or SEQ ID NO: 36, optionally wherein the polynucleotide encoding the IL-2 molecule comprises a nucleotide sequence having at least 85%, 90%, 95%, 96%, 97%, 98%, 99% or 100% identity to the sequence of SEQ ID NO: 36, optionally wherein the polynucleotide encoding the IL-2 molecule comprises the nucleotide sequence of SEQ ID NO: 36.

16. The LNP composition of any one of claims 1 to 15, wherein the IL-2 molecule comprises a half-life extender, e.g., a protein (or fragment thereof) that binds to a serum protein such as albumin, IgG, FcRn or transferrin.

17. The LNP composition of any one of claims 1 to 16, wherein the half-life extender comprises albumin or a fragment thereof, or an Fc domain of an antibody molecule (e.g., an Fc domain with enhanced FcRn binding).

18. The LNP composition of any one of claims 1 to 17, wherein the half-life extender is albumin, or a fragment thereof.

19. The LNP composition of any one of claims 1 to 18, wherein the half-life extender is albumin, e.g., human serum albumin (HSA), mouse serum albumin (MSA), cyno serum albumin (CSA) or rat serum albumin (RSA).

20. The LNP composition of claim 19, wherein the albumin is HSA comprising an amino acid sequence having at least 85%, 90%, 95%, 96%, 97%, 98%, 99% or 100% identity to the amino acid sequence of SEQ ID NO: 8.

21. The LNP composition of any one of claims 1 to 20, wherein the IL-2 molecule comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without the leader sequence, optionally wherein the IL-2 molecule comprises an amino acid sequence having at least 85%, 90%, 95%, 96%, 97%, 98%, 99% or 100% identity to the amino acid sequence of SEQ ID NO: 11 with or without the leader sequence.

22. The LNP composition of any one of claims 1 to 21, wherein the IL-2 molecule comprises the amino acid sequence of SEQ ID NO: 9, SEQ ID NO: 10, SEQ ID NO: 11, SEQ ID NO: 12 or SEQ ID NO: 13 with or without the leader sequence.

23. The LNP composition of claim 22, wherein the IL-2 molecule comprises the amino acid sequence of SEQ ID NO: 11.

24. The LNP composition of any one of claims 1 to 23, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 25;
(b) the nucleotide sequence of SEQ ID NO: 25; or
(c) the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

25. The LNP composition of any one of claims 1 to 23, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 36;
(b) the nucleotide sequence of SEQ ID NO: 36; or
(c) the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

26. The LNP composition of any one of claims 1 to 25, wherein the IL-2 molecule further comprises a targeting moiety, e.g., a T regulatory cell targeting moiety or a tissue-specific targeting moiety.

27. The LNP composition of claim 26, wherein the tissue-specific targeting moiety binds to ROS-CII, EDA, EDB, TnC A1, SyETP, GLUT-2, GD2, FAP, VCAM or MADCAM.

28. The LNP composition of claim 26, wherein the T regulatory cell targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

29. The LNP composition of claim 28, wherein the T regulatory cell targeting moiety binds to a molecule present on a T regulatory cell.

30. The LNP composition of claim 28 or 29, wherein the T regulatory cell targeting moiety comprises an antibody molecule that binds to CTLA-4, GITR, TLR8, or Nrp1.

31. The LNP composition of any one of claims 28-30, wherein the T regulatory cell targeting moiety comprises an antibody molecule that binds to CTLA-4.

32. The LNP composition of claim 31, wherein the targeting moiety comprising an antibody molecule that binds to CTLA-4 comprises an amino acid sequence having at least 85%, 90%, 95%, 96%, 97%, 98%, 99% or 100% identity to the amino acid sequence of SEQ ID NO: 17.

33. The LNP composition of any one of claims 25-32, wherein the IL-2 molecule comprising the targeting moiety comprises an amino acid sequence having at least 85%, 90%, 95%, 96%, 97%, 98%, 99% or 100% identity to the amino acid sequence of SEQ ID NO: 18, SEQ ID NO: 19, or SEQ ID NO: 20.

34. The LNP composition of any one of claims 25-33, wherein the IL-2 molecule comprising the targeting moiety is encoded by a nucleotide sequence having at least 85%, 90%, 95%, 96%, 97%, 98%, 99% or 100% identity to the nucleic acid sequence of SEQ ID NO: 21, SEQ ID NO:22 or SEQ ID NO: 23.

35. A lipid nanoparticle (LNP) composition comprising a polynucleotide comprising an mRNA which encodes a GM-CSF molecule comprising an amino acid sequence having at least 85%, 90%, 95%, 96%, 97%, 98%, 99% or 100% identity to the amino acid sequence of a GM-CSF molecule provided in Table 3A.

36. The LNP composition of claim 35, wherein the GM-CSF molecule comprises a naturally occurring GM-CSF molecule, a fragment of a naturally occurring GM-CSF molecule, or a variant thereof.

37. The LNP composition of claim 35 or 36, wherein the GM-CSF molecule comprises an amino acid sequence having at least 85%, 90%, 95%, 96%, 97%, 98%, 99% or 100% identity to the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.

38. The LNP composition of any one of claims 35-37, wherein the GM-CSF molecule comprises the amino acid sequence of SEQ ID NO: 14, SEQ ID NO: 188, SEQ ID NO: 39, SEQ ID NO: 41, SEQ ID NO: 43, SEQ ID NO: 16, SEQ ID NO: 200, SEQ ID NO: 205, SEQ ID NO: 210, SEQ ID NO: 215, or SEQ ID NO: 220.

39. The LNP composition of any one of claims 35-38, wherein the polynucleotide encoding the GM-CSF molecule comprises a nucleotide sequence having at least 85%, 90%, 95%, 96%, 97%, 98%, 99% or 100% identity to the nucleic acid sequence of SEQ ID NO: 15, SEQ ID NO: 38, SEQ ID NO: 40, SEQ ID NO: 42, SEQ ID NO: 44, SEQ ID NO: 24, SEQ ID NO: 201, SEQ ID NO: 206, SEQ ID NO: 211, SEQ ID NO: 216, SEQ ID NO: 221, SEQ ID NO: 204, SEQ ID NO: 209, SEQ ID NO: 214, SEQ ID NO: 219, or SEQ ID NO: 224,
optionally wherein polynucleotide encoding the GM-CSF molecule comprises:
(a) a nucleotide sequence having at least 85%, 90%, 95%, 96%, 97%, 98%, 99% or 100% identity to the sequence of SEQ ID NO: 221;
(b) the nucleotide sequence of SEQ ID NO: 221; or
(c) the nucleotide sequence of SEQ ID NO: 224 which consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 29.

40. The LNP composition of any one of claims 35-39, wherein the GM-CSF molecule comprises a half-life extender, e.g., a protein (or fragment thereof) that binds to a serum protein such as albumin, IgG, FcRn or transferrin.

41. The LNP composition of claim 40, wherein the half-life extender comprises albumin or a fragment thereof, or an Fc domain of an antibody molecule (e.g., an Fc domain with enhanced FcRn binding).

42. The LNP composition of claim 40 or 41, wherein the half-life extender is albumin, or a fragment thereof.

43. The LNP composition of any one of claims 40-42, wherein the half-life extender is albumin, e.g., human serum albumin (HSA), mouse serum albumin (MSA), cyno serum albumin (CSA) or rat serum albumin (RSA).

44. The LNP composition of claim 43, wherein the albumin is HSA comprising an amino acid sequence having at least 85%, 90%, 95%, 96%, 97%, 98%, 99% or 100% identity to the amino acid sequence of SEQ ID NO: 8.

45. The LNP composition of claim 43 or 44, wherein the GM-CSF molecule comprising HSA comprises an amino acid sequence having at least 85%, 90%, 95%, 96%, 97%, 98%, 99% or 100% identity to the amino acid sequence of SEQ ID NO: 16 or SEQ ID NO: 220.

46. The LNP composition of any one of claims 43-45, wherein the GM-CSF molecule comprising HSA is encoded by a nucleotide sequence having at least 85%, 90%, 95%, 96%, 97%, 98%, 99% or 100% identity to the nucleic acid sequence of SEQ ID NO: 24, SEQ ID NO: 221, or SEQ ID NO: 224.

47. The LNP composition of any one of claims 35-46, wherein the GM-CSF molecule further comprises a targeting moiety, e.g., a dendritic cell targeting moiety, or a tissue-specific targeting moiety.

48. The LNP composition of claim 47, wherein the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

49. A lipid nanoparticle (LNP) composition, comprising:
(a) a first polynucleotide encoding an IL-2 molecule; and
(b) a second polynucleotide encoding a GM-CSF molecule,
wherein (a) and (b) comprise an mRNA, and optionally wherein, the first and second polynucleotides are formulated at an (a):(b) mass ratio of 1:1.

50. A lipid nanoparticle (LNP) composition, for stimulating T regulatory cells, the LNP composition comprising:
(a) a first polynucleotide encoding an IL-2 molecule; and
(b) a second polynucleotide encoding a GM-CSF molecule,
wherein (a) and (b) comprise an mRNA.

51. The LNP composition of claim 49 or 50, wherein the IL-2 molecule comprises a naturally occurring IL-2 molecule, a fragment of a naturally occurring IL-2 molecule, or a variant thereof.

52. The LNP composition of any one of claims 49-51, wherein the IL-2 molecule comprises a variant of a naturally occurring IL-2 molecule (e.g., an IL-2 variant, e.g., as described herein), or a fragment thereof.

53. The LNP composition of any one of claims 49-52, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).

54. The LNP composition of any one of claims 49-53, wherein the GM-CSF molecule comprises a naturally occurring GM-CSF molecule, a fragment of a naturally occurring GM-CSF molecule, or a variant thereof.

55. The LNP composition of any one of the preceding claims, wherein the LNP composition increases the level and/or activity of T regulatory cells and/or suppressor T cells, e.g., as determined by an assay in a sample (e.g., a sample from a subject).

56. The LNP composition of claim 55, wherein the T regulatory cells comprise FoxP3+ expressing and/or CD25+ expressing T regulatory cells.

57. The LNP composition of claim 55 or 56, wherein the T regulatory cells are CD4+ and/or CD8+ T regulatory cells.

58. The LNP composition of any one of claims 55 to 57, wherein the increase in level and/or activity of T regulatory cells is compared to level and/or activity of T regulatory cells in an otherwise similar sample which is: not contacted with the LNP composition comprising (a) and (b); or contacted with a composition comprising only (a) or a composition comprising only (b).

59. The LNP composition of any one of claims 55 to 58, wherein the increase in level and/or activity of T regulatory cells occurs in vitro or in vivo.

60. The LNP composition of any one of claims 55 to 59, wherein the increase in level and/or activity of T regulatory cells comprises a one, or all or a combination (e.g., 2, 3, or all) of the following parameters:
(a) increased level of (e.g., number or proportion of) T regulatory cells (e.g., FoxP3+ T regulatory cells);
(b) increased activity of STAT5, e.g., STAT5 phosphorylation, in T regulatory cells (e.g., FoxP3+ T regulatory cells);
(c) increased activity or expression level of CTLA-4, TIGIT, ICOS and/or GITR in T regulatory cells (e.g., FoxP3+ T regulatory cells); and
(d) decreased activity or expression level of TGF beta and/or IL-10.

61. The LNP composition of claim 60, wherein the LNP composition increases the level of (e.g., number or proportion of) FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1-3, 8 or 11.

62. The LNP composition of claim 61, wherein the increase in the level of Fox P3+ T regulatory cells is compared to an otherwise similar population of cells not contacted with a composition comprising IL-2 and GM-CSF.

63. The LNP composition of claim 60, wherein the LNP composition increases in the activity of STAT5 (e.g., STAT5 phosphorylation) in FoxP3+ T regulatory cells, e.g., a 1.5 to 5 fold (e.g., 2 to 4 fold, 2 to 3 fold, 3 to 4 fold, or 3 to 5 fold) increase, as measured by an assay in Example 1.

64. The LNP composition of claim 63, wherein the increase in activity of STAT5 is compared to the activity of STAT5 in FoxP3− cells or Natural Killer cells.

65. The LNP composition of claim 60, wherein the LNP composition increases in the activity and/or expression level of one or more (e.g., two, three, or all) of CTLA-4, TIGIT, ICOS and/or GITR in T regulatory cells (e.g., FoxP3+ T regulatory cells), e.g., a 1.5 to 10 fold (e.g., 2 to 8 fold, 3 to 7 fold, 4 to 6 fold, 1.5 to 10 fold, 1.5 to 8 fold, 1.5 to 6 fold, 1.5 to 4 fold, 8 to 10 fold, 6 to 10 fold, or 4 to 10 fold) increase, as measured by an assay in Example 2.

66. The LNP composition of claim 65, wherein the increase in activity and/or expression level of one or more (e.g., two, three, or all) of CTLA-4, TIGIT, ICOS and/or GITR in T regulatory cells is compared to an otherwise similar population of cells not contacted with a composition comprising IL-2 and GM-CSF.

67. The LNP composition of any one of the preceding claims, wherein the composition increases T regulatory cells (e.g., CD25+ T regulatory cells) as compared to type 1 T helper cells (Tₕ1) cells; type 2 T helper cells (Tₕ2) cells; type 17 T helper cells (Tₕ17) cells and/or CD8+ T conventional cells (T con).

68. The LNP composition of claim 67, wherein the increase in level and/or activity of suppressor T cells is compared to level and/or activity of suppressor T cells in an otherwise similar sample which is: not contacted with the composition comprising (a) and (b); or contacted with a composition comprising only (a) or a composition comprising only (b).

69. The LNP composition of claim 68, wherein the increase in level and/or activity of suppressor T cells occurs in vitro or in vivo.

70. The LNP composition of claim 68 or 69, wherein the increase in level and/or activity of suppressor T cells comprises one or both of the following parameters:
(a) increased activity or expression level of Lag 3; and/or
(b) increased activity or expression level of CD94b.

71. A lipid nanoparticle comprising a polynucleotide encoding a molecule that stimulates T regulatory cells (e.g., an IL-2 molecule) for use, in the treatment of a disease associated with an aberrant T regulatory cell function in a subject.

72. A method of treating or preventing a disease associated with an aberrant T regulatory cell function in a subject, comprising administering to the subject an effective amount of a lipid nanoparticle comprising a polynucleotide encoding a molecule that stimulates T regulatory cells (e.g., an IL-2 molecule).

73. The LNP composition for use of claim 71, or the method of claim 72, further comprising administration of a lipid nanoparticle comprising a polynucleotide encoding a GM-CSF molecule.

74. The LNP composition for use of claim 71 or 73, or the method of claim 72 or 73, wherein the molecule that stimulates T regulatory cells comprises an IL-2 molecule, or a molecule that binds to a receptor present on T regulatory cells.

75. A lipid nanoparticle (LNP) comprising a polynucleotide encoding a molecule that stimulates dendritic cells (e.g., a GM-CSF molecule) for use, in the treatment of a disease associated with an aberrant T regulatory cell function in a subject.

76. A method of treating or preventing a disease associated with an aberrant T regulatory cell function in a subject, comprising administering to a subject an effective amount of a lipid nanoparticle comprising a polynucleotide encoding molecule that stimulates dendritic cells (e.g., a GM-CSF molecule).

77. The LNP composition for use of claim 75, or the method of claim 76, further comprising administration of a lipid nanoparticle comprising a polynucleotide encoding an IL-2 molecule.

78. The LNP composition for use, or the method of any one of claims 75-77 wherein the molecule that stimulates dendritic cells comprises:
(a) a molecule that stimulates, e.g., increases, the expression and/or level of TNFalpha, IL-10, CCL-2 and/or nitric oxide in dendritic cells; or
(b) a GM-CSF molecule.

79. The LNP composition for use, or the method of any one of claims 75-78, wherein the molecule that stimulates dendritic cells results in an increased level and/or activity of CD11b+ or CD11c+ dendritic cells.

80. The LNP composition for use, or the method of any one of claims 75-79, wherein administration of the LNP comprising the polynucleotide encoding the GM-CSF molecule results in a modulation of dendritic cell activity and/or modulation of expression and/or activity of myeloid cells in a sample from the subject.

81. The LNP composition for use, or the method of claim 80, wherein the sample has an increase in, e.g., increased number or proportion of, dendritic cells expressing CD11b and/or CD11c.

82. The LNP composition for use, or the method of claim 81, wherein the increase in DCs expressing CD11b (CD11b+ DCs) is at least 1.2-10 fold (e.g., at least 1.2, 1.5, 2, 3, 4, 5, 6, 7, 8, 9, or 10 fold), e.g., as compared to an otherwise similar sample not contacted with the LNP comprising the GM-CSF molecule, or contacted with a different LNP.

83. The LNP composition for use of any one of claims 73-74 or 77-82, or the method of any one of claims 73-74 or 77-82, wherein the LNP comprising a polynucleotide encoding an IL-2 molecule and the LNP comprising a polynucleotide encoding a GM-CSF molecule are administered sequentially or simultaneously, e.g., wherein the LNP encoding the IL-2 molecule is administered first and the LNP encoding GM-CSF molecule is administered second; or the LNP encoding the IL-2 molecule is administered second and the LNP encoding GM-CSF molecule is administered first.

84. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, in the treatment or prevention of a disease associated with an aberrant T regulatory cell function in a subject.

85. A method of treating or preventing a disease associated with an aberrant T regulatory cell function in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

86. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for inhibiting an immune response in a subject.

87. A method of inhibiting an immune response in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

88. A composition comprising a first lipid nanoparticle (LNP) comprising a first polynucleotide encoding an IL-2 molecule for use, in combination with a second lipid nanoparticle (LNP) comprising a second polynucleotide encoding a GM-CSF molecule, for stimulating T regulatory cells in a subject.

89. A method of stimulating T regulatory cells in a subject, comprising administering to the subject an effective amount of a first lipid nanoparticle comprising a first polynucleotide encoding an IL-2 molecule in combination with a second lipid nanoparticle comprising a second polynucleotide encoding a GM-CSF molecule.

90. The method or composition for use of any one of claims 84-89, wherein the first LNP and the second LNP are administered sequentially or simultaneously.

91. The method or composition for use of any one of claims 84-90, wherein the first LNP and the second LNP are administered in the same or in separate compositions.

92. The method or composition for use of any one of claims 71-91, wherein the LNP composition is administered by a route of administration chosen from: subcutaneous, intramuscular, intravenous, oral, intranasal, intraocular, or rectal, optionally wherein the LNP composition is administered by a subcutaneous route of administration.

93. The method or composition for use of any one of claims 71-74 or 84-92, wherein the IL-2 molecule comprises a naturally occurring IL-2 molecule, a fragment of a naturally occurring IL-2 molecule, or a variant thereof.

94. The method or composition for use of claim 93, wherein the IL-2 molecule comprises a variant of a naturally occurring IL-2 molecule (e.g., an IL-2 variant, e.g., as described herein), or a fragment thereof.

95. The method or composition for use of claim 93 or 94, wherein the IL-2 molecule comprising an IL-2 variant preferentially binds to an IL-2 receptor comprising an IL-2 receptor alpha chain (CD25), compared to an IL-2 receptor that does not comprise the IL-2 receptor alpha chain (CD25).

96. The method or composition for use of any one of claims 93-95, wherein the IL-2 molecule comprising an IL-2 variant has a higher affinity (e.g., at least 1.5 fold, 2 fold, 3 fold, 4 fold, 5 fold, or 10 fold higher) for an IL-2 receptor comprising an IL-2 receptor alpha chain (CD25), compared to a naturally occurring IL-2 molecule.

97. The method or composition for use of any one of claims 93-96, wherein the IL-2 variant comprises a mutation (e.g., substitution) in the IL-2 polypeptide sequence at any one, all or a combination (e.g., 2, 3, 4, 5, or more) of the following positions: amino acid 1, amino acid 4, amino acid 8, amino acid 10, amino acid 11, amino acid 13, amino acid 20, amino acid 26, amino acid 29, amino acid 30, amino acid 31, amino acid 35, amino acid 37, amino acid 46, amino acid 48, amino acid 49, amino acid 61, amino acid 64, amino acid 68, amino acid 69, amino acid 71, amino acid 74, amino acid 75, amino acid 76, amino acid 79, amino acid 88, amino acid 89, amino acid 90, amino acid 91, amino acid 92, amino acid 101, amino acid 103, amino acid 114, amino acid 125, amino acid 128, or amino acid 133.

98. The method or composition for use of any one of claims 93-97, wherein the IL-2 variant comprises any one, all or a combination (e.g., 2, 3, 4, 5, or more) of the following mutations (e.g., substitutions): A1T, S4P, K8R, T10A, Q11R, Q13R, D20T, N26D, N29S, N30S, Y31H, K35R, T37R, M46L, K48E, K49R, E61D, K64R, E68D, V69A, N71T, Q74P, S75P, K76R, H79R, N88D, I89V, N90H, V91K, I92T, T101A, F103S, I114V, C125S, I128T, or T133N.

99. The method or composition for use of any one of claims 71-74 or 84-98, wherein the IL-2 molecule comprises an amino acid sequence having at least 85%, 90%, 95%, 96%, 97%, 98%, 99% or 100% identity to the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.

100. The method or composition for use of any one of claims 71-74 or 84-99, wherein the IL-2 molecule comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 30, SEQ ID NO: 31, SEQ ID NO: 32, SEQ ID NO: 33, SEQ ID NO: 33, SEQ ID NO: 34, or SEQ ID NO: 35.

101. The method or composition for use of any one of claims 71-74 or 84-100, wherein the polynucleotide encoding the IL-2 molecule comprises a nucleotide sequence having at least 85%, 90%, 95%, 96%, 97%, 98%, 99% or 100% identity to the nucleotide sequence of SEQ ID NO: 7.

102. The method or composition for use of any one of claims 71-74 or 84-101, wherein the IL-2 molecule comprises a half-life extender, e.g., a protein (or fragment thereof) that binds to a serum protein such as albumin, IgG, FcRn or transferrin.

103. The method or composition for use of claim 102, wherein the half-life extender comprises albumin or a fragment thereof, or an Fc domain of an antibody molecule (e.g., an Fc domain with enhanced FcRn binding).

104. The method or composition for use of claim 102 or 103, wherein the half-life extender is albumin, or a fragment thereof.

105. The method or composition for use of any one of claims 102-104, wherein the half-life extender is albumin, e.g., human serum albumin (HSA), mouse serum albumin (MSA), cyno serum albumin (CSA) or rat serum albumin (RSA).

106. The method or composition for use of claim 105, wherein the albumin is HSA comprising an amino acid sequence having at least 85%, 90%, 95%, 96%, 97%, 98%, 99% or 100% identity to the amino acid sequence of SEQ ID NO: 8.

107. The method or composition for use of any one of claims 71-74 or 84-106, wherein the IL-2 molecule comprises an amino acid sequence having at least 85%, 90%, 95%, 96%, 97%, 98%, 99% or 100% identity to the amino acid sequence of any one of SEQ ID NO: 9, SEQ ID NO: 10, SEQ ID NO: 11, SEQ ID NO: 12 or SEQ ID NO: 13 with or without a leader sequence, optionally wherein the IL-2 molecule comprises an amino acid sequence having at least 85%, 90%, 95%, 96%, 97%, 98%, 99% or 100% identity to the amino acid sequence of SEQ ID NO: 11 with or without the leader sequence.

108. The method or composition for use of claim 107, wherein the IL-2 molecule comprises the amino acid sequence of SEQ ID NO: 9, SEQ ID NO: 10, SEQ ID NO: 11, SEQ ID NO: 12 or SEQ ID NO: 13 with or without a leader sequence.

109. The method or composition for use of claim 107 or 108, wherein the IL-2 molecule comprises the amino acid sequence of SEQ ID NO: 11.

110. The method or composition for use of any one of claims 107-109, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 25;
(b) the nucleotide sequence of SEQ ID NO: 25; or
(c) the nucleotide sequence of SEQ ID NO: 28 which consists from 5′ to 3′ end: 5′ UTR of SEQ ID NO: 26, ORF sequence of SEQ ID NO: 25, 3′ UTR of SEQ ID NO: 27 and Poly A tail of SEQ ID NO: 29.

111. The method or composition for use of any one of claims 107-109, wherein the polynucleotide encoding the IL-2 molecule comprises:
(a) a nucleotide sequence having at least 85%, 90%, 95%, 96%, 97%, 98%, 99% or 100% identity to the sequence of SEQ ID NO: 36;
(b) the nucleotide sequence of SEQ ID NO: 36; or
(c) the nucleotide sequence of SEQ ID NO: 37 which consists from 5′ to 3′ end: 5′ UTR of SEQ ID NO: 26, ORF sequence of SEQ ID NO: 36, 3′ UTR of SEQ ID NO: 27 and Poly A tail of SEQ ID NO: 29.

112. The method or composition for use of any one of claims 71-74 or 84-111, wherein the IL-2 molecule further comprises a targeting moiety, e.g., a T regulatory cell targeting moiety or a tissue-specific targeting moiety.

113. The method or composition for use of any one of claims 75-92, wherein the GM-CSF molecule comprises a naturally occurring GM-CSF molecule, a fragment of a naturally occurring GM-CSF molecule, or a variant thereof.

114. The LNP composition, LNP composition for use, or the method of any one of the preceding claims, wherein the polynucleotide encoding the IL-2 molecule (e.g., the first polynucleotide), or the polynucleotide encoding the GM-CSF molecule (e.g., the second polynucleotide), or both, comprises at least one chemical modification.

115. The LNP composition of claim 114, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

116. The LNP composition of claim 115, wherein the chemical modification is selected from the group consisting of pseudouridine, N1-methylpseudouridine, 5-methylcytosine, 5-methoxyuridine, and a combination thereof.

117. The LNP composition of claim 115, wherein the chemical modification is N1-methylpseudouridine.

118. The LNP composition, LNP composition for use, or the method of any one of the preceding claims, wherein each mRNA in the lipid nanoparticle comprises fully modified N1-methylpseudouridine.

119. The LNP composition, LNP composition for use, or the method of any one of the preceding claims, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

120. The LNP composition, method or composition for use of claim 119, wherein the ionizable lipid is an amino lipid, e.g., as described herein.

121. The LNP composition, method or composition for use of claim 119 or 120, wherein the ionizable lipid comprises Compound 18 or Compound 25.

122. The LNP composition, method or composition for use of any one of claims 119-121, wherein the PEG-lipid is PEG-DMG or Compound P-428.

123. The LNP composition, method or composition for use of any one of claims 119-121, wherein the sterol lipid is cholesterol.

124. The LNP composition, method or composition for use of any one of claims 119-123, wherein the phospholipid is DSPC.

125. The LNP composition, method or composition for use of claim 119 or 120, wherein the ionizable lipid comprises Compound 18, the PEG-lipid is Compound P-428, the sterol lipid is cholesterol and the phospholipid is DSPC.

126. The LNP composition, method or composition for use of claim 119 or 120, wherein the ionizable lipid comprises Compound 25, the PEG-lipid is Compound P-428, the sterol lipid is cholesterol and the phospholipid is DSPC.

127. The LNP composition, method or composition for use of any one of claims 119-126, wherein the LNP comprises a molar ratio of about 50% ionizable lipid (e.g., Compound 18 or Compound 25):about 10% phospholipid:about 38.5% cholesterol; and about 1.5% PEG lipid.

128. The LNP composition, method or composition for use of any one of claims 119-126, wherein the LNP comprises a molar ratio of about 47.5 mol % of ionizable lipid (e.g., Compound 18 or Compound 25):about 10.5 mol % non-cationic helper lipid or phospholipid:about 39 mol % sterol or other structural lipid:and about 3.0 mol % PEG lipid.

129. The LNP composition, method or composition for use of any one of claims 119-126, wherein the LNP comprises a molar ratio of about 47.5 mol % of ionizable lipid comprising Compound 18:about 10.5 mol % DSPC as the non-cationic helper lipid or phospholipid:about 39 mol % cholesterol as the sterol or other structural lipid:and about 3.0 mol % of compound P-428 as the PEG lipid.

130. The LNP composition, method or composition for use of any one of claims 119-126, wherein the LNP comprises a molar ratio of about 47.5 mol % of ionizable lipid comprising Compound 25:about 10.5 mol % DSPC as the non-cationic helper lipid or phospholipid:about 39 mol % cholesterol as the sterol or other structural lipid:and about 3.0 mol % of compound P-428 as the PEG lipid.

131. The LNP composition, method or composition for use of any one of claims 119-126, wherein the LNP comprises a molar ratio of about 45% to about 50% ionizable lipid (e.g., Compound 18 or Compound 25):about 5% to about 15% phospholipid:about 30% to about 40% cholesterol:and about 1% to about 5% PEG lipid.
